(19) United States
(12) Patent Application Publication
(10) Pub. No.: US 2026/0078134 A1
(43) Pub. Date: Mar. 19, 2026

(54) Macrocyclic Compounds as RAS Inhibitors

(71) Applicant: BeOne Medicines I GmbH, Basel (CH)

(72) Inventors: Jing Li, Beijing (CN); Yian Guo, Beijing (CN); Sanjia Xu, Beijing (CN); Zhiwei Wang, Beijing (CN)

(73) Assignee: BeOne Medicines I GmbH, Basel (CH)

(21) Appl. No.: 19/332,430

(22) Filed: Sep. 18, 2025

(30) Foreign Application Priority Data
Sep. 19, 2024 (WO) PCT/CN2024/119750
Mar. 20, 2025 (WO) PCT/CN2025/083786
Jul. 24, 2025 (WO) PCT/CN2025/110338

(51) Int. Cl. C07D 513/22; A61K 31/504; A61K 31/5377; A61K 31/5383; A61K 31/5386; A61K 31/551; C07D 519/00

(52) U.S. Cl. CPC C07D 513/22; A61K 31/504; A61K 31/5377; A61K 31/5383; A61K 31/5386; A61K 31/551; C07D 519/00

(57) ABSTRACT

Provided herein are compounds having the following structure:

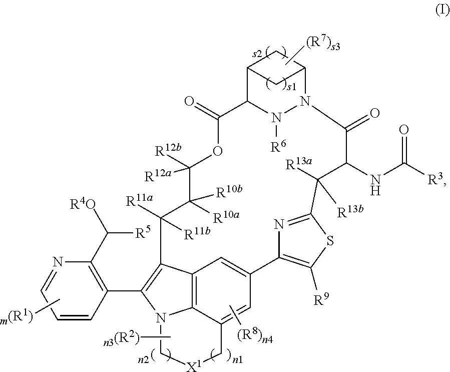

or pharmaceutically acceptable salts, hydrates, or solvates thereof, wherein the substituents are as defined herein, their pharmaceutical compositions and uses for modulating the activity of multiple RAS mutants.

### CROSS-REFERENCE TO RELATED APPLICATIONS

This application claims priority to International Application No. PCT/CN2024/119750, filed Sep. 19, 2024, International Application No. PCT/CN2025/083786, filed Mar. 20, 2025, and International Application No. PCT/CN2025/110338, filed Jul. 24, 2025, the contents of each of which are hereby incorporated by reference in their entireties.

### FIELD

Provided herein are macrocyclic compounds that inhibit the activity of multiple RAS mutants as well as their pharmaceutical compositions and methods of use.

### BACKGROUND

Rat sarcoma (RAS) genes are one of the major oncogenic drivers in various aggressive malignancies. Comprising three main isozymes, kirsten-RAS (KRAS), neuroblastoma-RAS (NRAS), and harvey-RAS (HRAS), RAS genes along with their downstream signaling cascades regulate a myriad of cellular processes, such as cell proliferation, survival, migration, etc. As a result, aberrations in RAS genes collectively exhibit mutations in approximately one-fifth of patients with a broad spectrum of cancers [Prior. I. A., Cancer Research, 2020, 80 (14): pp. 2969-74], such as non-small-cell lung cancer (NSCLC), pancreatic ductal adenocarcinoma (PDAC), colorectal cancer (CRC), melanoma, and certain hematological malignancies [Moore, A. R., Nature Reviews Drug Discovery, 2020, 19: pp. 533-52].

Historically, RAS proteins have been deemed ‘intractable’ due to several challenging characteristics. Specifically, the extremely high affinity of RAS for GTP, the abundant presence of GTP within cells, and the absence of accessible binding pockets on RAS proteins have posed significant obstacles to developing effective therapeutics [Yang, H., Cancer Communication, 2023, 43 (1): pp. 42-74]. This previously undruggable target was not vindicated until the FDA approval of sotorasib and adagrasib, marking a milestone and opening new avenues for exploring RAS-related treatment options.

While the KRAS^{G12C}-selective inhibitors have demonstrated their proof-of-concept succuss in clinic, there is still a significant unmet medical need for effective treatments for RAS mutations beyond KRAS^{G12C}. Furthermore, the emergence of acquired mutation following the use of allele-selective inhibitors potentially leads to drug resistance and curtail their therapeutic utility. Therefore, it is highly desirable to pursue strategies that can concurrently inhibit multiple irregular RAS signaling with one single agent, which could also be compatible with various combinatorial strategies [Perureana, N., Nature Reviews Cancer, 2024, 24, pp. 316-37].

In pursuit of prolonged clinical benefits and enhanced therapeutic potential, our efforts have been directed towards the invention of novel cyclic compounds. These compounds are designed to address multiple RAS deregulations and ultimately to overcome cancers driven by RAS mutations.

### SUMMARY

Provided herein is a compound of Formula (I):

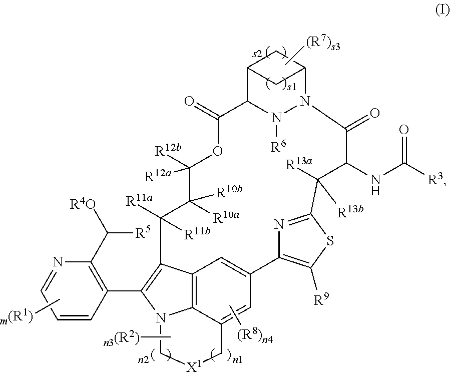

or a hydrate, solvate, N-oxide, pharmaceutically acceptable salt, stereoisomer, tautomer, or deuterated analog thereof, wherein
X¹ is —O—, —S— or —CH₂—;
n1 is 0, 1 or 2;
n2 is 1, 2 or 3;
n3 is 0, 1, 2, 3 or 4;
n4 is 0, 1 or 2;
s1 and s2 are each independently 1 or 2;
s3 is 0, 1 or 2;
m is 1, 2 or 3;
R¹ is selected from hydrogen, halogen, substituted or unsubstituted —C₁₋₆alkyl, substituted or unsubstituted —C₃₋₈cycloalkyl, substituted or unsubstituted 3- to 10-membered heterocyclyl, substituted or unsubstituted —C₆-C₁₂aryl, substituted or unsubstituted 5- to 12-membered heteroaryl, —OR^{12a}, —NR^{1a}R^{1b}, —COR^{1a}, —CO₂R^{1a} and —CONR^{1a}R^{1b};
R^{1a} and R^{1b} are each independently selected from hydrogen, substituted or unsubstituted —C₁₋₆alkyl, substituted or unsubstituted —C₃-C₆cycloalkyl, substituted or unsubstituted 3- to 12-membered heterocyclyl, substituted or unsubstituted —C₆-C₁₂aryl and substituted or unsubstituted 5- to 12-membered heteroaryl;
R² is each independently selected from hydrogen, halogen, substituted or unsubstituted —C₁₋₆alkyl, substituted or unsubstituted —C₃₋₆cycloalkyl, substituted or unsubstituted 3- to 6-membered heterocyclyl, substituted or unsubstituted —C₆-C₁₂aryl, substituted or unsubstituted 5- to 12-membered heteroaryl, —OR^{2a}, —NR^{2a}R^{2b}, —COR^{2a}, —CO₂R^{2a} and —CONR^{2a}R^{2b}; or
when adjacent or geminal, two R² together with the atom(s) to which they are attached, form a 3- to 6-membered substituted or unsubstituted ring, said ring comprising 0-3 heteroatoms independently selected from nitrogen, oxygen and sulfur;
R^{2a} and R^{2b} are each independently selected from hydrogen, substituted or unsubstituted —C₁₋₆alkyl, substituted or unsubstituted —C₃-C₆cycloalkyl, substituted or unsubstituted 3- to 12-membered heterocyclyl, substituted or unsubstituted —C₆-C₁₂aryl and substituted or unsubstituted 5- to 12-membered heteroaryl;
R³ is selected from substituted or unsubstituted —C₁₋₆alkyl, substituted or unsubstituted —C₃₋₆cycloalkyl, substituted or unsubstituted 3- to 12-membered heterocyclyl, substituted or unsubstituted —C₆-C₁₂aryl, substituted or unsubstituted 5- to 12-membered heteroaryl, —OR, and —NR^{3a}R^{3b};
R^{3a} and R^{3b} are each independently selected from hydrogen, substituted or unsubstituted —C₁₋₆alkyl, substituted or unsubstituted —C₃-C₆cycloalkyl, substituted or unsubstituted 3- to 12-membered heterocyclyl, substituted or unsubstituted —C₆-C₁₂aryl and substituted or unsubstituted 5- to 12-membered heteroaryl;
R⁴ is selected from substituted or unsubstituted —C₁₋₃alkyl, substituted or unsubstituted —C₃₋₆cycloalkyl, substituted or unsubstituted 3- to 6-membered heterocyclyl, substituted or unsubstituted —C₆-C₁₂aryl and substituted or unsubstituted 5- to 12-membered heteroaryl;
R⁵ is selected from hydrogen, halogen, substituted or unsubstituted —C₁₋₃alkyl, substituted or unsubstituted —C₃₋₆cycloalkyl, substituted or unsubstituted 3- to 6-membered heterocyclyl, substituted or unsubstituted —C₆-C₁₂aryl and substituted or unsubstituted 5- to 12-membered heteroaryl;
R⁶ is selected from hydrogen and substituted or unsubstituted —C₁₋₃alkyl;
R⁷, R⁸ and R⁹ are each independently selected from hydrogen, halogen, substituted or unsubstituted —C₁₋₆alkyl and —CN;
R^{10a} and R^{10b} are each independently selected from hydrogen and substituted or unsubstituted —C₁₋₃ alkyl; or
R^{10a} and R^{10b} together with the atom to which they are attached, form a 3- to 6-membered substituted or unsubstituted ring, said ring comprising 0-3 heteroatoms independently selected from nitrogen, oxygen and sulfur;
R^{11a}, R^{11b}, R^{12a}, R^{12b}, R^{13a} and R^{13b} are each independently selected from hydrogen, halogen and substituted or unsubstituted —C₁₋₃alkyl.

Also provided herein is a pharmaceutical composition comprising a compound of Formula (I) or a pharmaceutically acceptable salt, hydrate, solvate, tautomer, or deuterated analog thereof and a pharmaceutically acceptable excipient.

Also provided herein is a method for inhibiting the activity of multiple RAS mutants in a cell, comprising contacting said cell with a therapeutically effective amount of a compound of Formula (I), or a pharmaceutically acceptable salt, hydrate, solvate, tautomer, or deuterated analog thereof.

Also provided herein is a method for treating cancer comprising administering to a subject in need thereof a therapeutically effective amount of a compound of formula (I), or a pharmaceutically acceptable salt, hydrate, solvate, tautomer, or deuterated analog thereof.

### DETAILED DESCRIPTION

### Definitions

Unless specifically defined elsewhere in this document, all other technical and scientific terms used herein have the meaning commonly understood by one of ordinary skill in the art.

It is meant to be understood that proper valences are maintained for all moieties and combinations thereof, that monovalent moieties having more than one atom are drawn from left to right and are attached through their left ends, and that divalent moieties are also drawn from left to right.

It is also meant to be understood that a specific embodiment of a variable moiety may be the same or different as another specific embodiment having the same identifier.

The indefinite articles “a” and “an” and the definite article “the” include plural as well as single referents, unless the context clearly indicates otherwise.

“Alkenyl” means a straight or branched hydrocarbon chain having from 2 to 12 carbon atoms and including at least one carbon-carbon double bond. Typically the alkenyl group has from 2 to 8 carbon atoms, preferably from 2 to 6 carbon atoms, and more preferably from 2 to 4 carbon atoms. Representative alkenyl groups include, but are not limited to, -vinyl, -allyl, -1-butenyl, -2-butenyl, -isobutylenyl, -1-pentenyl, 2pentenyl, -3-methyl-1-butenyl, -2-methyl-2-butenyl, -2,3-dimethyl-2-butenyl, -1-hexenyl, 2-hexenyl, -3-hexenyl, -1-heptenyl, -2-heptenyl, -3-heptenyl, -1-octenyl, -2-octenyl, -3-octenyl and the like.

“Alkenylene” means a bivalent straight or branched hydrocarbon radical derived from an alkenyl, as defined above, by removing one hydrogen from a carbon of the alkenyl. The bi-valency can be on the same carbon. Representative alkenylene groups include, but are not limited to, —CH═CH—, —CH₂—CH═CH—, —CH₂—CH═CH—CH₂— and the like.

“Alkyl” means a saturated straight or branched hydrocarbon chain having from 1 to 12 carbon atoms, typically from 1 to 8 carbon atoms, preferably from 1 to 6 carbon atoms, and more preferable from 1 to 4 carbon atoms. Representative alkyl groups include, but are not limited to, -methyl, -ethyl, -n-propyl, -n-butyl, -n-pentyl, -n-hexyl, -isopropyl, -sec-butyl, -isobutyl, tert-butyl, -isopentyl, -neopentyl, tert-pentyl, -2methylpentyl, -3methylpentyl, 4methylpentyl, 2,3dimethylbutyl and the like.

“Alkylene” means a bivalent straight or branched hydrocarbon radical derived from an alkyl, as defined above, by removing one hydrogen from a carbon of the alkyl. The bi-valency can be on the same carbon. Representative alkylene groups include, but are not limited to, —CH₂—, —CH₂CH₂—, —CH(CH₃)—, —(CH₂)₅—, and the like.

“Alkynyl” means a straight or branched hydrocarbon chain having from 2 to 12 carbon atoms and including at least one carbon-carbon triple bonds. Typically the alkynyl group has from 2 to 8 carbon atoms, preferably from 2 to 6 carbon atoms, and more preferable from 2 to 4 carbon atoms. Representative alkynyl groups include, but are not limited to, ethynyl, propynyl, and butynyl.

“Alkynylene” means a bivalent straight or branched hydrocarbon radical derived from an alkynyl, as defined above, by removing one hydrogen from a carbon of the alkynyl. The bi-valency can be on the same carbon. Representative alkynylene groups include, but are not limited to, —C≡C—, —CH₂—C≡C—, —CH₂—C≡C—CH₂— and the like.

“Alkoxy” or “alkoxyl” means —O-alkyl, wherein alkyl is defined herein.

“Amino” means a radical of the formula: —NH₂.

“Aryl” means an aromatic carbocyclic group having from 6 to 14 ring carbon atoms and having a single ring (e.g., phenyl) or multiple fused rings (e.g., naphthyl or anthryl). Preferably, the aryl group is phenyl or naphthyl, and more preferably phenyl.

“Atropisomer” means a stereoisomer resulting from hindered rotation about a single bond axis where the rotational barrier is high enough to allow for the isolation of the individual rotational isomer.

The term “axial chirality” is used to refer to stereoisomerism resulting from the non-planar arrangement of four groups in pairs about a chirality axis. It is exemplified by allenes abC═C═Ccd (or abC═C═Cab) and by the atropisomerism of ortho-substituted biphenyls.

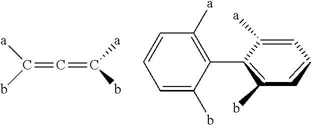

The configuration in molecular entities possessing axial chirality is specified by the stereodescriptors Rₐ and Sₐ (or by M or P).

In the context of the compounds disclosed herein, the term “M-isomer” refers to the configuration represented by the following compound:

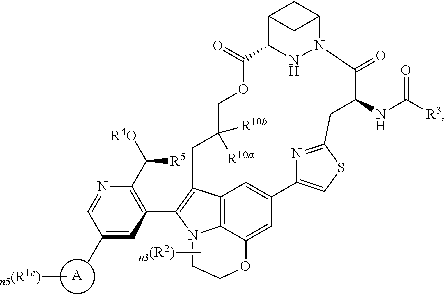

or its equivalent.

In the context of the compounds disclosed herein, the term “P-isomer” refers to the configuration represented by the following compound:

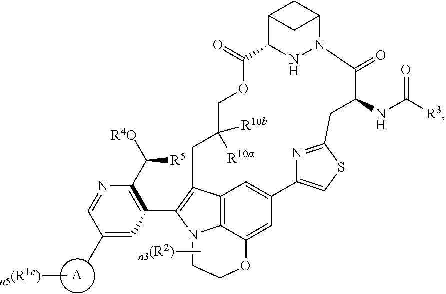

or its equivalent.

It is meant to be understood that the

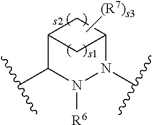

moiety is

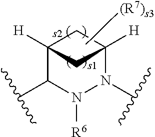

configuration unless the context clearly indicates otherwise.

“Bridged ring” means a ring system formed by connecting two non-adjacent atoms (usually carbon atoms) of a ring (mono- or fused multi cyclic) with an atom or chain of atoms. These two non-adjacent atoms are called “bridgeheads”. A bridged ring system may include only carbon atoms as ring atoms or may include carbon atoms and one to four heteroatoms (i.e., N, O, or S) as ring atoms. Examples of bridged ring systems include 1-bicyclo[1.1.1]pentyl, bicyclo[2.1.1]hexyl, bicyclo[2.2.1]heptyl, bicyclo[2.2.2]octyl, adamantanyl, norbornanyl, bicyclo [3.2.1] octyl, bicyclo [2.2.2] octyl, bicyclo[3.3.1] nonyl, bicyclo[3.2.2] nonyl, 2-oxa-bicyclo [2.2.2] octyl, 1-aza-bicyclo [2.2.2] octyl, 3-aza-bicyclo [3.2.1] octyl, and 2, 6-dioxa-tricyclo [3.3.1.0^{3,7}] nonyl.

“Carboxy” means a radical of the formula: C(═O)OH.

“Cycloalkyl” means a saturated, monocyclic or multiple fused ring system having from 3 to 14 ring carbon atoms and no heteroatoms. Typically, the cycloalkyl group is a monocyclic or bicyclic group having 3 to 10 ring carbon atoms, preferably a monocyclic group having 3 to 8 ring carbon atoms, more preferably a monocyclic group having 3 to 6 ring carbon atoms, and even more preferably a monocyclic group having 5 to 6 ring carbon atoms. Representative cycloalkyl groups include, but are not limited to cyclopropyl, cyclobutyl, cyclopentyl, cyclohexyl, cycloheptyl, and cyclooctyl.

“Cycloalkenyl” means a non-aromatic, unsaturated, monocyclic or multiple fused ring systems group having from 3 to 14 ring carbon atoms and no ring heteroatoms, and having at least one carbon-carbon double bond. Typically, the cycloalkenyl group is a monocyclic or bicyclic group having 3 to 10 ring carbon atoms, preferably a monocyclic group having 3 to 8 ring carbon atoms, more preferably a monocyclic group having 3 to 6 ring carbon atoms, and even more preferably a monocyclic group having 5 to 6 ring carbon atoms. Representative cycloalkenyl groups include, but are not limited to, cyclohexenyl, cyclopentenyl, cyclohexadienyl, butadienyl, pentadienyl, hexadienyl, 1,2,3,4-tetrahydronaphthyl.

“Cycloalkynyl” means a non-aromatic, unsaturated, monocyclic or multiple fused ring system having from 8 to 14 ring carbon atoms and no ring heteroatoms and including at least one carbon-carbon triple bond. Typically, the cycloalkynyl group is a monocyclic group having 8 to 10 ring carbon atoms, preferably 9 to 10 ring carbon atoms. Representative cycloalkynyl groups include, but are not limited to, cyclooctynyl, cyclononynyl, and cyclodecenyl.

“Halogen” means fluorine, chlorine, bromine or iodine.

“Heteroaryl” means an aromatic monocyclic or multiple fused ring system in which at least one, typically one to four, preferably one to three, more preferably one to two, or even more preferably one of the ring carbon atoms are independently replaced with a heteroatom selected from the group consisting of O, S and N. Typically, the heteroaryl group is a monocyclic or bicyclic ring system having 5 to 14 ring atoms, preferably a monocyclic or bicyclic ring system having 5 to 10 ring atoms, and more preferably a monocyclic ring having 5 to 6 ring atoms. Heteroaryl groups can be bonded to other groups at any ring atom (i.e., at any carbon atom or heteroatom of the heteroaryl ring system), provided that proper valences are maintained. Representative heteroaryl groups include, but are not limited to, pyrrolyl, pyrazolyl, imidazolyl, triazolyl, tetrazolyl, oxazolyl, isoxazolyl, benzisoxazolyl (e.g., benzo[d]isoxazolyl), thiazolyl, pyrolyl, pyridazinyl, pyrimidyl, pyrazinyl, thiophenyl, benzothiophenyl, furanyl, benzofuranyl, indolyl (e.g., indolyl-2-onyl or isoindolin-1-onyl), azaindolyl (pyrrolopyridyl or 1H-pyrrolo[2,3-b]pyridyl), indazolyl, benzimidazolyl (e.g., 1Hbenzo[d]imidazolyl), imidazopyridyl (e.g., azabenzimidazolyl or 1H-imidazo[4,5-b]pyridyl), pyrazolopyridyl, triazolopyridyl, benzotriazolyl (e.g., 1H-benzo[d][1,2,3]triazolyl), benzoxazolyl (e.g., benzo[d]oxazolyl), benzothiazolyl, benzothiadiazolyl, isoxazolopyridyl, thianaphthalenyl, purinyl, quinolinyl, isoquinolinyl, quinoxalinyl, and quinazolinyl groups.

“Heterocyclyl” means a non-aromatic monocyclic or multiple fused ring system in which at least one, preferably one to four, more preferably one to three, or even more preferably one to two of the ring carbon atoms are independently replaced with a heteroatom selected from the group consisting of O, S and N, wherein the nitrogen and sulfur heteroatoms may optionally be oxidized and/or the nitrogen heteroatom may be optionally be quarternized. The heterocyclyl group can be saturated or unsaturated. Typically, the heterocyclyl group is a monocyclic or bicyclic ring system having 3 to 14 ring atoms, preferably a monocyclic or bicyclic ring system having 3 to 10 ring atoms, more preferably a monocyclic ring having 3 to 6 ring atoms and even more preferably a monocyclic ring having 5 to 6 atoms. ring atoms The heterocyclyl group can be bonded to other groups at any ring atom (i.e., at any carbon atom or heteroatom of the heterocyclic ring system), provided that proper valences are maintained. Representative heterocyclyl groups include, but are not limited to, aziridinyl, azetidinyl, azepanyl, oxetanyl, pyrrolidyl, imidazolidinyl (e.g., imidazolidin-4-onyl or imidazolidin-2,4-dionyl), pyrazolidinyl, thiazolidinyl, tetrahydrothiophenyl, tetrahydrofuranyl, dioxolyl, pyrrolinyl, imidazolinyl, pyrazolinyl, thiazolinyl, piperidyl, piperazinyl (e.g., piperazin-2-onyl), morpholinyl, thiomorpholinyl, tetrahydropyranyl (e.g., tetrahydro-2H-pyranyl), tetrahydrothiopyranyl, oxathianyl, dioxyl, dithianyl, pyranyl, dihydropyridyl, dihydrodithiinyl, dihydrodithionyl, 1,4-dioxaspiro[4.5]decanyl, 2-oxo-1-oxa-3,8-diazaspiro[4.5]decane, 1-oxo-2,8-diazaspiro[4.5]decane, 3-oxo-2,8-diazaspiro[4.5]decane, 3-oxo-1-oxa-4,9-diazaspiro[5.5]undecane, 2-oxo-1-oxa-3,9-diazaspiro[5.5]undecane, homopiperazinyl, quinuclidyl, indolinyl, isoindolinyl, quinolizinyl, dihydrobenzothiazinyl, dihydrobenzofuranyl, dihydroindolyl, dihydrobenzodioxinyl, tetrahydroquinolinyl groups, and the like.

“Hydrate” means a solvate wherein the solvent molecule is H₂O.

“Oxo” means ═O wherein the double bond is attached to a carbon, sulfur, or nitrogen.

“Pharmaceutically acceptable” means suitable for use in contact with the tissues of human beings without excessive toxicity, irritation, allergic response, or other problem or complication, and commensurate with a reasonable benefit/risk ratio.

“Pharmaceutically acceptable excipient” means a pharmaceutically acceptable inert ingredient or vehicle, such as a liquid or solid filler, diluent, excipient, solvent or encapsulating material. Each excipient must be “acceptable” in the sense of being compatible with the other ingredients of the formulation and not deleterious to the recipients thereof.

“Pharmaceutically acceptable salt(s)” means a salt prepared from a pharmaceutically acceptable non-toxic acid or base including an inorganic acid and base and an organic acid and base.

“Solvate” means a physical association of a compound of formula (I) or a pharmaceutically acceptable salt thereof with one or more pharmaceutically acceptable solvent molecules. This physical association involves varying degrees of ionic and covalent bonding, including hydrogen bonding.

“Spiro ring” means a ring system that contains a bicyclic ring sharing a single ring atom (usually a quaternary carbon atom) between two ring members. Typically the spiro ring is a bicyclic ring system. The individual rings within a spiro ring system may be identical or different, may contain carbon atoms only as ring atoms, or may contain one to four heteroatoms as ring atom(s). Examples of spiro ring systems include spiropentane, spirohexane, spiro[5.4]decane, spiro[4.3]octane, spiro[5.2]octane, and spiropyran.

“Stereoisomer” or “stereomerically pure” means one stereoisomer of a compound that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound.

“Subject” means a mammal. In some preferred embodiment, “Subject” means a human.

“Substituted” means that any one or more hydrogens on the designated atom or group is replaced with a selection (i.e., substituent) from the indicated groups, provided that the designated atom's normal valence is not exceeded. It will be understood by those skilled in the art, with respect to any group containing one or more substituents, that such groups are not intended to introduce any substitution or substitution patterns that are sterically impractical, synthetically non-feasible and/or inherently unstable.

“Tautomer” means an isomeric form of a compound that is in equilibrium with one or more other isomers due to a tautomeric shift, resulting in different arrangements of atoms and chemical bonds. The concentration of the isomeric form will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

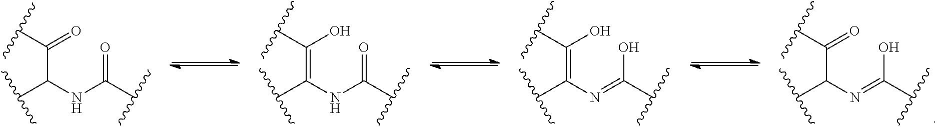 [SMILES: *C(=O)NC(*)C(*)=O ; *C(=O)N/C(*)=C(/*)O ; */C(O)=N/C(*)=C(/*)O ; *C(=O)C(*)/N=C(/*)O]

“Therapeutically effective amount” means an amount capable of treating a disorder, disease or condition, or symptoms thereof, disclosed herein.

The terms “treating”, “treatment” and the like, shall include the management and care of a subject or patient, preferably a mammal, more preferably a human, for the purpose of combating a disease, condition, or disorder and includes the administration of a compound of the present invention to prevent the onset of the symptoms or complications, alleviate the symptoms or complications, slow the progression of the disease or disorder, or eliminate the disease, condition, or disorder. The terms “treating” or“treatment” further include: (a) inhibiting the disease-state, i.e., arresting its development, and/or (b) relieving the disease-state, i.e., causing regression of the disease state.

### Compounds

One objective of the present invention is to provide compounds and derivatives which function as RAS inhibitors, and methods of preparation and uses thereof.

The following aspects and embodiments are not intended to be an explicit or implicit admission that these aspects or embodiments are independent or distinct nor should it be interpreted as such. Rather, it is intended to convey information so that the full breadth of the present disclosure can be understood. Furthermore, the following aspects and embodiments are not meant to be limiting on the full breadth of the disclosure as recited herein.

Aspect 1. A compound of Formula (I):

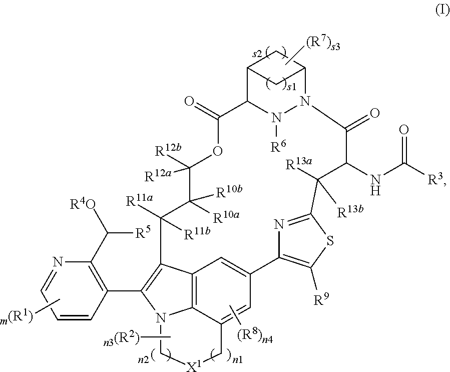

or a hydrate, solvate, N-oxide, pharmaceutically acceptable salt, stereoisomer, tautomer, or deuterated analog thereof, wherein:
X¹ is —O—, —S— or —CH₂—;
n is 0, 1 or 2;
n2 is 1, 2 or 3;
n3 is 0, 1, 2, 3 or 4;
n4 is 0, 1 or 2;
ss and s2 are each independently 1 or 2;
s3 is 0, 1 or 2;
m is 1, 2 or 3;
R¹ is selected from hydrogen, halogen, substituted or unsubstituted —C₁₋₆alkyl, substituted or unsubstituted —C₃₋₈cycloalkyl, substituted or unsubstituted 3- to 10-membered heterocyclyl, substituted or unsubstituted —C₆-C₁₂aryl, substituted or unsubstituted 5- to 12-membered heteroaryl, —OR^{1a}, —NR^{1a}R^{1b}, —COR^{1a}, —CO₂R^{1a}, and —CONR^{1a}R^{1b};
R^{1a} and R^{1b} are each independently selected from hydrogen, substituted or unsubstituted —C₁₋₆alkyl, substituted or unsubstituted —C₃-C₆cycloalkyl, substituted or unsubstituted 3- to 12-membered heterocyclyl, substituted or unsubstituted —C₆-C₁₂aryl, and substituted or unsubstituted 5- to 12-membered heteroaryl;
R² is each independently selected from hydrogen, halogen, substituted or unsubstituted —C₁₋₆alkyl, substituted or unsubstituted —C₃₋₆cycloalkyl, substituted or unsubstituted 3- to 6-membered heterocyclyl, substituted or unsubstituted —C₆-C₁₂aryl, substituted or unsubstituted 5- to 12-membered heteroaryl, —OR^{2a}, —NR^{2a}R^{2b}, —COR^{2a}, —CO₂R^{2a}, and —CONR^{2a}R^{2b}; or
when adjacent or geminal, two R² taking together with the atom(s) to which they are attached, form a 3- to 6-membered substituted or unsubstituted ring, said ring comprising 0-3 heteroatoms independently selected from nitrogen, oxygen, and sulfur;
R^{2a} and R^{2b} are each independently selected from hydrogen, substituted or unsubstituted —C₁₋₆alkyl, substituted or unsubstituted —C₃-C₆cycloalkyl, substituted or unsubstituted 3- to 12-membered heterocyclyl, substituted or unsubstituted —C₆-C₁₂aryl, and substituted or unsubstituted 5- to 12-membered heteroaryl;
R³ is selected from substituted or unsubstituted —C₁₋₆alkyl, substituted or unsubstituted —C₃₋₆ cycloalkyl, substituted or unsubstituted 3- to 12-membered heterocyclyl, substituted or unsubstituted —C₆-C₁₂aryl, substituted or unsubstituted 5- to 12-membered heteroaryl, —OR^{3a}, and —NR^{3a}R^{3b};
R^{3a} and R^{3b} are each independently selected from hydrogen, substituted or unsubstituted —C₁₋₆alkyl, substituted or unsubstituted —C₃-C₆cycloalkyl, substituted or unsubstituted 3- to 12-membered heterocyclyl, substituted or unsubstituted —C₆-C₁₂aryl, and substituted or unsubstituted 5- to 12-membered heteroaryl;
R⁴ is selected from substituted or unsubstituted —C₁₋₃alkyl, substituted or unsubstituted —C₃₋₆ cycloalkyl, substituted or unsubstituted 3- to 6-membered heterocyclyl, substituted or unsubstituted —C₆-C₁₂aryl, and substituted or unsubstituted 5- to 12-membered heteroaryl;
R⁵ is selected from hydrogen, halogen, substituted or unsubstituted —C₁₋₃alkyl, substituted or unsubstituted —C₃₋₆cycloalkyl, substituted or unsubstituted 3- to 6-membered heterocyclyl, substituted or unsubstituted —C₆-C₁₂aryl, and substituted or unsubstituted 5- to 12-membered heteroaryl; R⁶ is selected from hydrogen and substituted or unsubstituted —C₁₋₃alkyl;
R⁷, R⁸ and R⁹ are each independently selected from hydrogen, halogen, substituted or unsubstituted —C₁₋₆alkyl, and —CN;
R^{10a} and R^{10b} are each independently selected from hydrogen and substituted or unsubstituted —C₁₋₃alkyl; or
R^{10a} and R^{10b} taking together with the atom to which they are attached, form a 3- to 6-membered substituted or unsubstituted ring, said ring comprising 0-3 heteroatoms independently selected from nitrogen, oxygen, and sulfur;
R^{11a}, R^{12b}, R^{12a}, R^{12b}, R^{13a} and R^{13b} are each independently selected from hydrogen, halogen, and substituted or unsubstituted —C₁₋₃alkyl.

Aspect 2. The compound of Aspect 1, wherein the compound is formula (Ia):

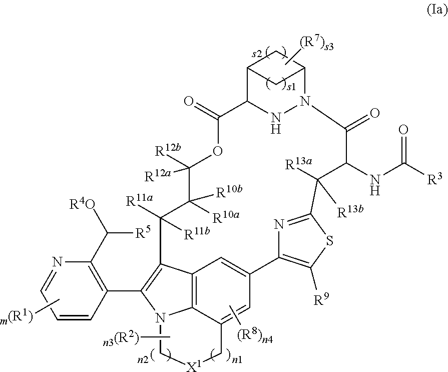

or a hydrate, solvate, N-oxide, pharmaceutically acceptable salt, stereoisomer, tautomer, or deuterated analog thereof, wherein:
X¹ is —O— or —CH₂—;
n1 is 0, 1 or 2;
n2 is 1, 2 or 3;
n3 is 0, 1 or 2;
n4 is 0 or 1;
s1 and s2 are each independently 1 or 2;
s3 is 0 or 1;
m is 1 or 2;
R¹ is selected from hydrogen, halogen, —C₁₋₆alkyl, —C₃₋₈cycloalkyl, 3- to 10-membered heterocyclyl, —OR^{1a}, —NR^{1a}R^{1b}, —COR^{1a}, —CO₂R^{1a} and —CONR^{1a}R^{1b}, wherein each of said —C₁₋₆alkyl, —C₃₋₈cycloalkyl and 3- to 10-membered heterocyclyl is optionally substituted with at least one substituent selected from R^{1c};
R^{1a} and R^{1b} are each independently selected from hydrogen, —C₁₋₆alkyl, —C₃-C₆cycloalkyl, 3- to 12-membered heterocyclyl, —C₆-C₁₂aryl and 5- to 12-membered heteroaryl, wherein each of said —C₁₋₆alkyl, —C₃-C₆cycloalkyl, 3- to 12-membered heterocyclyl, —C₆-C₁₂aryl and 5- to 12-membered heteroaryl is optionally substituted with at least one substituent R^{1d};
R^{1c} and R^{1d} are each independently selected from hydrogen, halogen, —C₁₋₆alkyl, —C₂₋₆-alkynyl, —C₃-C₆cycloalkyl, 3- to 6-membered heterocyclyl, —C₆-C₁₂aryl, 5- to 12-membered heteroaryl, —OR^{1e}, —COR^{1e}, —CO₂R^{1e}, —CONR^{1e}R^{1f}, —NR^{1e}R^{1f}, —NR^{1e}COR^{1f}, —NR^{1e}CO₂R^{1f}, oxo and —CN, wherein each of said —C₁₋₆alkyl, —C₂₋₆alkynyl, —C₃-C₆cycloalkyl, 3- to 6-membered heterocyclyl, —C₆-C₁₂aryl and 5- to 12-membered heteroaryl is optionally substituted with at least one substituent R^{1j}; or
two R^{1c} together with the atom(s) to which they are attached and any intervening atoms, form a 3- to 6-membered unsaturated or saturated ring, said ring comprising 0-3 heteroatoms independently selected from nitrogen, oxygen and sulfur and said ring is optionally substituted with at least one substituent selected from halogen, —C₁₋₆alkyl, —C₂₋₆alkynyl, —C₁₋₆alkoxy, —C₃-C₆cycloalkyl, 3- to 6-membered heterocyclyl, —C₆-C₁₂aryl, 5- to 12-membered heteroaryl, —CN, —OH, —NH₂ and oxo;
R^{1e} and R^{1f} are each independently selected from hydrogen, —C₁₋₆alkyl, —C₃-C₆cycloalkyl, 3- to 12-membered heterocyclyl, —C₆-C₁₂aryl and 5- to 12-membered heteroaryl, wherein each of said —C₁₋₆alkyl, —C₃-C₆cycloalkyl, 3- to 12-membered heterocyclyl, —C₆-C₁₂aryl and 5- to 12-membered heteroaryl is optionally substituted with at least one substituent selected from halogen, —C₁₋₆alkoxy, —C₃-C₆cycloalkyl, 3- to 6-membered heterocyclyl, —C₆-C₁₂aryl, 5- to 12-membered heteroaryl, —CN, —OH, —NH₂ and oxo;
R^{1j} is selected from halogen, —C₁₋₆alkyl, —C₂₋₆alkynyl, —C₁₋₆alkoxy, —C₃-C₆cycloalkyl, 3- to 6-membered heterocyclyl, —C₆-C₁₂aryl, 5- to 12-membered heteroaryl, —CN, —OH, —NH₂ and oxo; wherein each of said —C₁₋₆alkyl, —C₂₋₆-alkynyl, —C₁₋₆alkoxy, —C₃-C₆cycloalkyl, 3- to 6-membered heterocyclyl, —C₆-C₁₂aryl, 5- to 12-membered heteroaryl is optionally substituted with at least one substituent selected from —NH₂, —OH, alkynyl;
R² is each independently selected from hydrogen, halogen, —C₁₋₆alkyl, —C₃₋₆cycloalkyl, 3- to 6-membered heterocyclyl, —C₆-C₁₂aryl, 5- to 12-membered heteroaryl, —OR^{2a}, —NR^{2a}R^{2b}, —COR^{2a}, —CO₂R^{2a} and —CONR^{2a}R^{2b}, wherein each of said —C₁₋₆alkyl, —C₃₋₆cycloalkyl, 3- to 6-membered heterocyclyl, —C₆-C₁₂aryl and 5- to 12-membered heteroaryl is optionally substituted with at least one substituent R^{2e}; or
when adjacent or geminal, two R² together with the atom(s) to which they are attached, form a 3- to 6-membered ring, said ring comprising 0-3 heteroatoms independently selected from nitrogen, oxygen and sulfur, said ring is optionally substituted with at least one substituent R²;
R^{2a} and R^{2b} are each independently selected from hydrogen, —C₁₋₆alkyl, —C₃-C₆cycloalkyl, 3- to 12-membered heterocyclyl, —C₆-C₁₂aryl and 5- to 12-membered heteroaryl, wherein each of said —C₁₋₆alkyl, —C₃-C₆cycloalkyl, 3- to 12-membered heterocyclyl, —C₆-C₁₂aryl and 5- to 12-membered heteroaryl is optionally substituted with at least one substituent selected from halogen, —C₁₋₆alkoxy, —C₃-C₆cycloalkyl, 3- to 6-membered heterocyclyl, —C₆-C₁₂aryl, 5- to 12-membered heteroaryl, —CN, —OH, —NH₂ and oxo;
R^{2c} is each independently selected from hydrogen, halogen, —C₁₋₆alkyl, —C₃-C₆cycloalkyl, 3- to 6-membered heterocyclyl, —C₆-C₁₂aryl, 5- to 12-membered heteroaryl, —OR^{2d}, —COR^{2d}, —CO₂R^{2d}, —CONR^{2d}R^{2e}, —NR^{2d}R^{2e}, —NR^{2d}COR^{2e}, —NR^{2d}CO₂R^{2e}, oxo and —CN;
R^{2d} and R^{2e} are each independently selected from hydrogen, —C₁₋₆alkyl, —C₃-C₆cycloalkyl, 3- to 12-membered heterocyclyl, —C₆-C₁₂aryl and 5- to 12-membered heteroaryl, wherein each of said —C₁₋₆alkyl, —C₃-C₆cycloalkyl, 3- to 12-membered heterocyclyl, —C₆-C₁₂aryl and 5- to 12-membered heteroaryl is optionally substituted with at least one substituent selected from halogen, —C₁₋₆-alkoxy, —C₃-C₆cycloalkyl, 3- to 6-membered heterocyclyl, —C₆-C₁₂aryl, 5- to 12-membered heteroaryl, —CN, —OH, —NH₂ and oxo;
R³ is selected from —C₁₋₆alkyl, —C₃₋₆cycloalkyl, 3- to 12-membered heterocyclyl, —C₆-C₁₂aryl, 5- to 12-membered heteroaryl, —OR^{3a} and —NR^{3a}R^{3b}, wherein each of said —C₁₋₆alkyl, —C₃₋₆cycloalkyl, 3- to 12-membered heterocyclyl, —C₆-C₁₂aryl and 5- to 12-membered heteroaryl is optionally substituted with at least one substituent R^{3c};
R^{3a} and R^{3b} are each independently selected from hydrogen, —C₁₋₆alkyl, —C₃-C₆cycloalkyl, 3- to 12-membered heterocyclyl, —C₆-C₁₂aryl and 5- to 12-membered heteroaryl, wherein each of said —C₁₋₆alkyl, —C₃-C₆cycloalkyl, 3- to 12-membered heterocyclyl, —C₆-C₁₂aryl and 5- to 12-membered heteroaryl is optionally substituted with at least one substituent R^{d};
R^{3c} and R^{3d} are each independently selected from hydrogen, halogen, —C₁₋₆alkyl, —C₃-C₆cycloalkyl, 3- to 6-membered heterocyclyl, —C₆-C₁₂aryl, 5- to 12-membered heteroaryl, —OR^{3e}, —COR^{3e}, —CO₂R^{3e}, —CONR^{3e}R^{3f}, —NR^{3e}R^{3f}, —NR^{3e}COR^{3f}, —NR^{3e}CO₂R^{3f}, oxo and —CN; wherein each of said —C₁₋₆alkyl, —C₃-C₆cycloalkyl, 3- to 6-membered heterocyclyl, —C₆-C₁₂aryl and 5- to 12-membered heteroaryl is optionally substituted with at least one substituent selected from halogen, —C₁₋₆alkoxy, —C₃-C₆cycloalkyl, 3- to 6-membered heterocyclyl, —C₆-C₁₂aryl, 5- to 12-membered heteroaryl, —CN, —OH, —NH₂ and oxo;
R^{3e} and R^{3f} are each independently selected from hydrogen, —C₁₋₆alkyl, —C₃-C₆cycloalkyl, 3- to 12-membered heterocyclyl, —C₆-C₁₂aryl and 5- to 12-membered heteroaryl, wherein each of said —C₁₋₆alkyl, —C₃-C₆cycloalkyl, 3- to 12-membered heterocyclyl, —C₆-C₁₂aryl and 5- to 12-membered heteroaryl is optionally substituted with at least one substituent selected from halogen, —C₁₋₆alkoxy, —C₃-C₆cycloalkyl, 3- to 6-membered heterocyclyl, —C₆-C₁₂aryl, 5- to 12-membered heteroaryl, —CN, —OH, —NH₂ and oxo;
R⁴ is selected from —C₁₋₃alkyl, —C₃₋₆cycloalkyl, 3- to 6-membered heterocyclyl, —C₆-C₁₂aryl and 5- to 12-membered heteroaryl, wherein each of said —C₁₋₃alkyl, —C₃₋₆cycloalkyl, 3- to 6-membered heterocyclyl, —C₆-C₁₂aryl and 5- to 6-membered heteroaryl is optionally substituted with at least one substituent selected from R⁴;
R^{4a} is independently selected from hydrogen, halogen, —C₁₋₃alkyl, —C₃-C₆cycloalkyl, 3- to 6-membered heterocyclyl, —C₆-C₁₂aryl, 5- to 12-membered heteroaryl, —OR^{4b}, —COR^{4b}, —CO₂R^{4b}, —CONR^{4b}R^{4c}, —NR^{4b}R^{4c}, —NR^{4b}COR^{4c}, —NR^{4b}CO₂R^{4c}, oxo and —CN;
R^{4b} and R^{4c} are each independently selected from hydrogen, —C₁₋₆alkyl, —C₃-C₆cycloalkyl, 3- to 12-membered heterocyclyl, —C₆-C₁₂aryl and 5- to 12-membered heteroaryl, wherein each of said —C₁₋₆alkyl, —C₃-C₆cycloalkyl, 3- to 12-membered heterocyclyl, —C₆-C₁₂aryl and 5- to 12-membered heteroaryl is optionally substituted with at least one substituent selected from halogen, —C₁₋₆alkoxy, —C₃-C₆cycloalkyl, 3- to 6-membered heterocyclyl, —C₆-C₁₂aryl, 5- to 12-membered heteroaryl, —CN, —OH, —NH₂ and oxo;
R⁵ is selected from hydrogen, halogen, —C₁₋₃alkyl, —C₃₋₆cycloalkyl, 3- to 6-membered heterocyclyl, —C₆-C₁₂aryl and 5- to 12-membered heteroaryl, wherein each of said —C₁₋₃alkyl, —C₃₋₆cycloalkyl, 3- to 6-membered heterocyclyl, —C₆-C₁₂aryl and 5- to 12-membered heteroaryl is optionally substituted with at least one substituent R^{5a};
R^{5a} is independently selected from hydrogen, halogen, —C₁₋₃alkyl, —C₃-C₆cycloalkyl, 3- to 6-membered heterocyclyl, —C₆-C₁₂aryl, 5- to 12-membered heteroaryl, —OR^{5b}, —COR^{5b}, —CO₂R^{5b}, —CONR^{5b}R^{5c}, —NR^{5b}R^{5c}, —NR^{5b}COR^{5c}, —NR^{5b}CO₂R^{5c}, oxo and —CN;
R^{5b} and R^{5c} are each independently selected from hydrogen, —C₁₋₆alkyl, —C₃-C₆cycloalkyl, 3- to 12-membered heterocyclyl, —C₆-C₁₂aryl and 5- to 12-membered heteroaryl, wherein each of said —C₁₋₆alkyl, —C₃-C₆cycloalkyl, 3- to 12-membered heterocyclyl, —C₆-C₁₂aryl and 5- to 12-membered heteroaryl is optionally substituted with at least one substituent selected from halogen, —C₁₋₆alkoxy, —C₃-C₆cycloalkyl, 3- to 6-membered heterocyclyl, —C₆-C₁₂aryl, 5- to 12-membered heteroaryl, —CN, —OH, —NH₂ and oxo;
R⁷, R⁸ and R⁹ are each independently selected from hydrogen, halogen, —C₁₋₆alkyl and —CN, wherein said —C₁₋₆alkyl is optionally substituted with at least one substituent selected from the group consisting of halogen, —C₁₋₆alkoxy and —OH;
R^{10a} and R^{10b} are each independently selected from hydrogen and —C₁₋₃alkyl, wherein said —C₁₋₃alkyl is optionally substituted with at least one substituent selected from halogen, —C₁₋₃alkoxy, —C₃-C₆cycloalkyl, —CN, —OH, —NH₂ and oxo; or
R^{10a} and R^{10b} together with the atom to which they are attached, form a 3- to 6-membered ring, said ring comprising 0-3 heteroatoms independently selected from nitrogen, oxygen and sulfur, said ring is optionally substituted with at least one substituent selected from halogen, —C₁₋₃alkoxy, —C₃-C₆cycloalkyl, —CN, —OH, —NH₂ and oxo;
R^{11a}, R^{11b}, R^{12a}, R^{12b}, R^{13a} and R^{13b} are each independently selected from hydrogen, halogen and —C₁₋₃ alkyl, wherein said —C₁₋₃alkyl is optionally substituted with at least one substituent selected from hydrogen, halogen, —OH and —C₁₋₃alkoxy.

Aspect 3. The compound of Aspect 1 or 2, wherein the compound is formula (II)

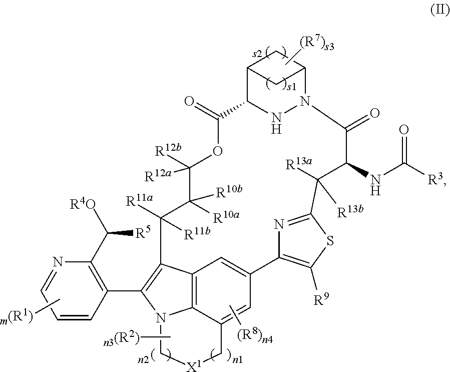

or a hydrate, solvate, N-oxide, pharmaceutically acceptable salt, stereoisomer, tautomer, or deuterated analog thereof.

In one embodiment, the compound is

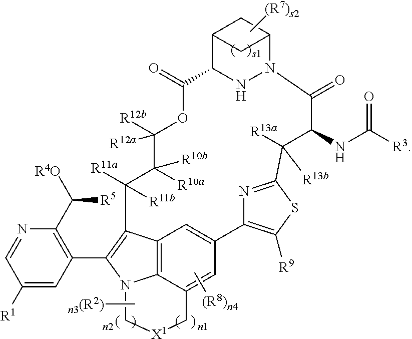

Aspect 4. The compound of Aspect 1 or 2, wherein the compound is formula (III)

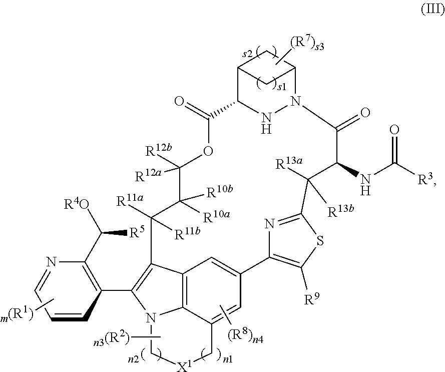

or a hydrate, solvate, N-oxide, pharmaceutically acceptable salt, stereoisomer, tautomer, or deuterated analog thereof.

In a preferred embodiment, the compound is

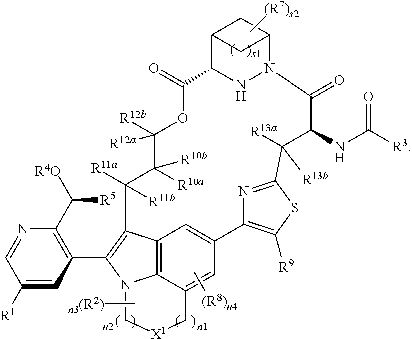

In one embodiment, the compound is

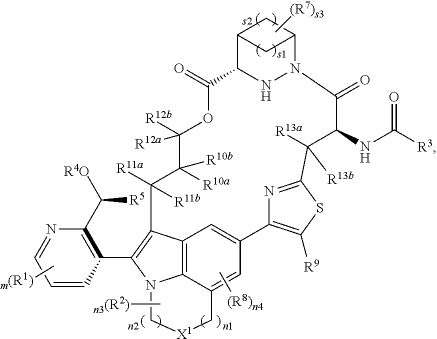

or a hydrate, solvate, N-oxide, pharmaceutically acceptable salt, stereoisomer, tautomer, or deuterated analog thereof, each of R¹, R², R³, R⁴, R⁵, R⁷, R⁸, R⁹, R^{10a}, R^{10b}, R^{11a}, R^{11b}, R^{12a}, R^{12b}, R^{13a}, R^{13b}, X¹, m, n1, n2, n3, n4, s1, s2 and s3 is defined as above.

In a preferred embodiment, the compound is

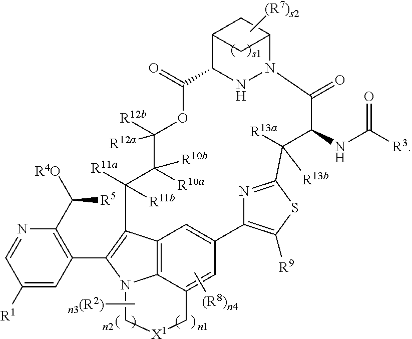

Aspect 5. The compound of Aspect 1 or 2, wherein the compound is formula (IV)

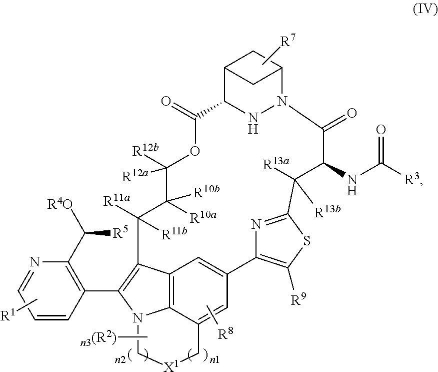

or a hydrate, solvate, N-oxide, pharmaceutically acceptable salt, stereoisomer, tautomer, or deuterated analog thereof.

In one embodiment, the compound is

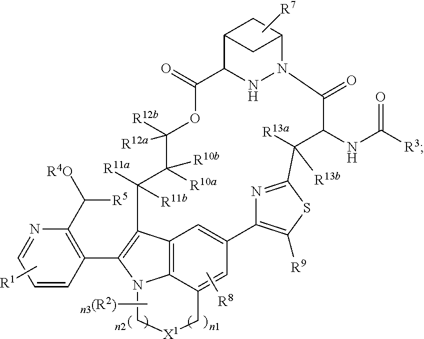

or a hydrate, solvate, N-oxide, pharmaceutically acceptable salt, stereoisomer, tautomer, or deuterated analog thereof, each of R¹, R², R³, R⁴, R⁵, R⁷, R⁸, R⁹, R^{10a}, R^{10b}, R^{11a}, R^{11b}, R^{12a}, R^{12b}, R^{13a}, R^{13b}, X¹, n1, n2 and n3 is defined as above.

In one embodiment, the compound is

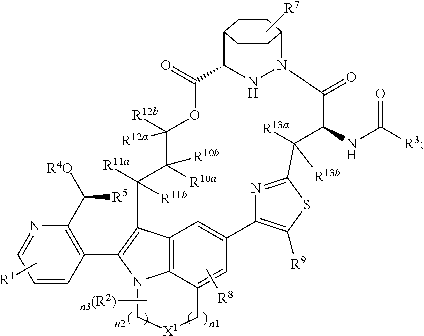

or a hydrate, solvate, N-oxide, pharmaceutically acceptable salt, stereoisomer, tautomer, or deuterated analog thereof, each of R¹, R², R³, R⁴, R⁵, R⁷, R⁸, R⁹, R^{10a}, R^{10b}, R^{11a}, R^{11b}, R^{12a}, R^{12b}, R^{13a}, R^{13b}, X¹, n1, n2 and n3 is defined as above.

Aspect 6. The compound of Aspect 1 or 2, wherein the compound is formula (V)

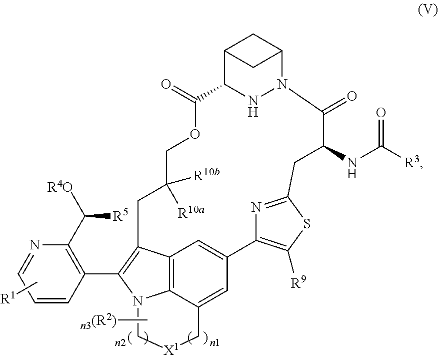

or a hydrate, solvate, N-oxide, pharmaceutically acceptable salt, stereoisomer, tautomer, or deuterated analog thereof.

In one embodiment, the compound is

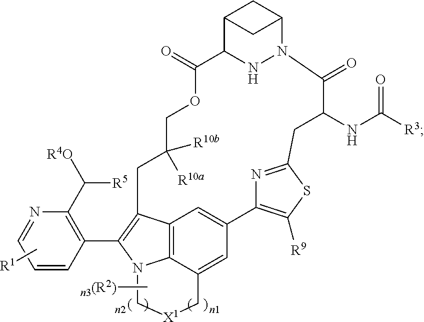

or a hydrate, solvate, N-oxide, pharmaceutically acceptable salt, stereoisomer, tautomer, or deuterated analog thereof, each of R¹, R³, R⁴, R⁵, R⁹, R^{10a}, R^{10b}, X¹, n1, n2 and n3 is defined as above.

In a preferred embodiment, the compound is

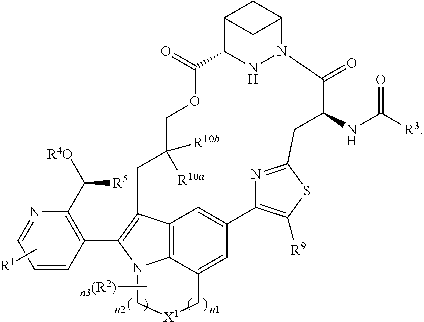

In one embodiment, the compound is

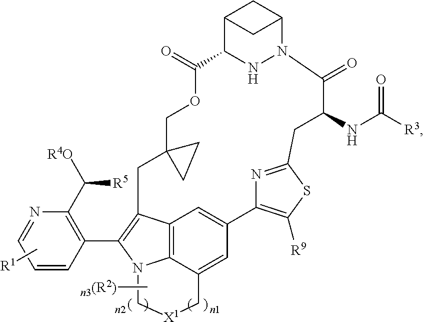

or a hydrate, solvate, N-oxide, pharmaceutically acceptable salt, stereoisomer, tautomer, or deuterated analog thereof, each of R¹, R², R³, R⁴, R⁵, R⁹, X¹, n1, n2 and n3 is defined as above.

In a preferred embodiment, the compound is

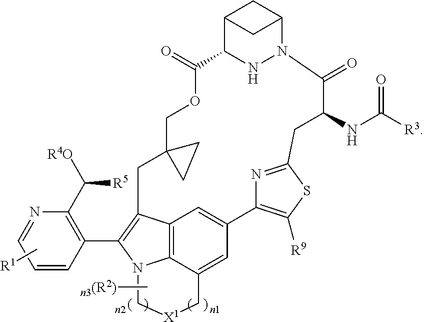

Aspect 7. The compound of Aspect 1 or 2, wherein the compound is formula (VI)

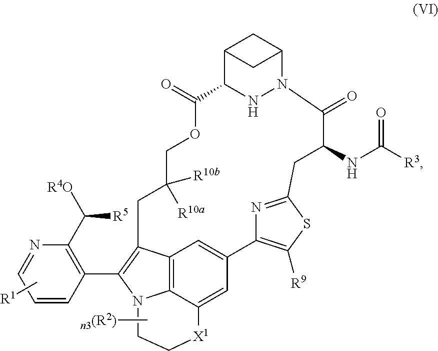

or a hydrate, solvate, N-oxide, pharmaceutically acceptable salt, stereoisomer, tautomer, or deuterated analog thereof.

In one embodiment, the compound is

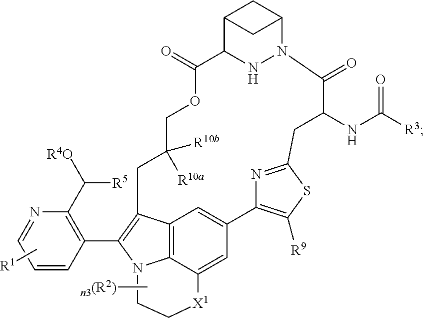

or a hydrate, solvate, N-oxide, pharmaceutically acceptable salt, stereoisomer, tautomer, or deuterated analog thereof, each of R¹, R², R³, R⁴, R⁵, R⁹, R^{10a}, R^{10b}, X¹ and n3 is defined as above.

In a preferred embodiment, the compound is

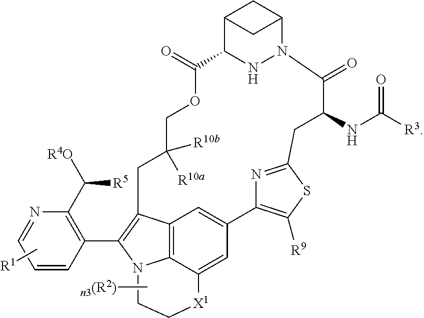

Aspect 8. The compound of Aspect 1 or 2, wherein the compound is formula (VIIa)

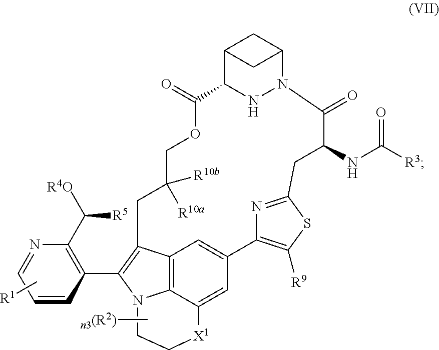

or a hydrate, solvate, N-oxide, pharmaceutically acceptable salt, stereoisomer, tautomer, or deuterated analog thereof.

In one embodiment, the compound is

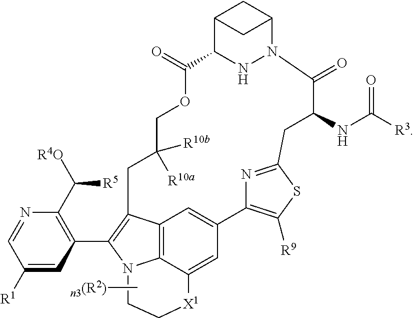

Aspect 9. The compound of Aspect 1 or 2, wherein the compound is formula (VIII)

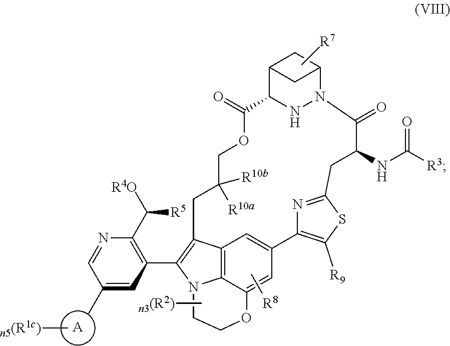

or a hydrate, solvate, N-oxide, pharmaceutically acceptable salt, stereoisomer, tautomer, or deuterated analog thereof;
Ring A is 5- to 10-membered heterocyclyl comprising 1 or 2 heteroatoms selected from N and O; and
n5 is 0, 1, 2, 3 or 4.

In one embodiment, the compound is

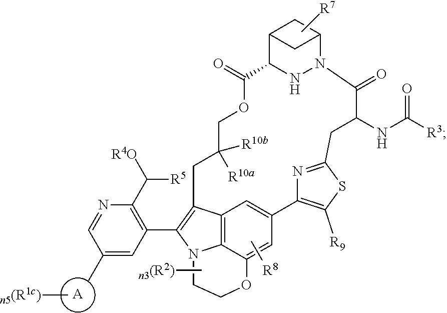

or a hydrate, solvate, N-oxide, pharmaceutically acceptable salt, stereoisomer, tautomer, or deuterated analog thereof;
Ring A is 5- to 10-membered heterocyclyl comprising 1 or 2 heteroatoms selected from N and O; and
n5 is 0, 1, 2, 3 or 4;
each of R^{1c}, R², R³, R⁴, R⁵, R⁷, R⁹, R^{10a}, R^{10b}, X¹ and n3 is defined as above.

Aspect 10. The compound of Aspect 1 or 2, wherein the compound is formula (IXa) or (IXb)

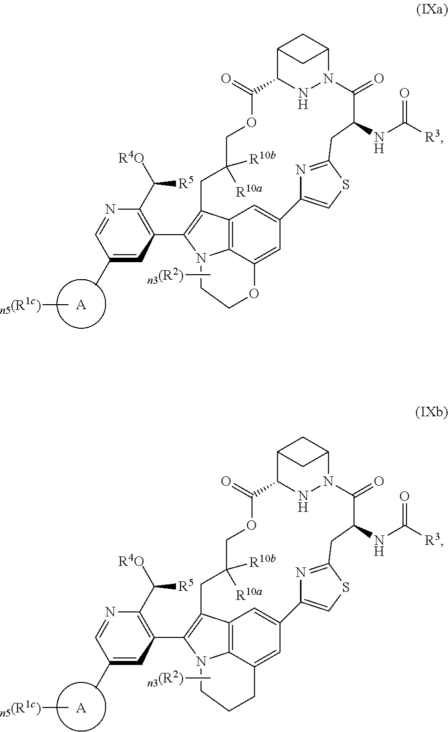

or a hydrate, solvate, N-oxide, pharmaceutically acceptable salt, stereoisomer, tautomer, or deuterated analog thereof;
Ring A is 5- to 10-membered heterocyclyl comprising 1 or 2 heteroatoms selected from N and O; and
n5 is 0, 1, 2, 3 or 4.

In one embodiment, the compound is

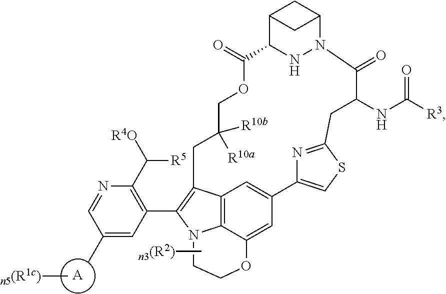

or a hydrate, solvate, N-oxide, pharmaceutically acceptable salt, stereoisomer, tautomer, or deuterated analog thereof;
Ring A is 5- to 10-membered heterocyclyl comprising 1 or 2 heteroatoms selected from N and O; and
n5 is 0, 1, 2, 3 or 4;
each of R^{1c}, R², R³, R⁴, R⁵, R^{10a}, R^{10b} and n3 is defined as above.

In one embodiment, the compound is

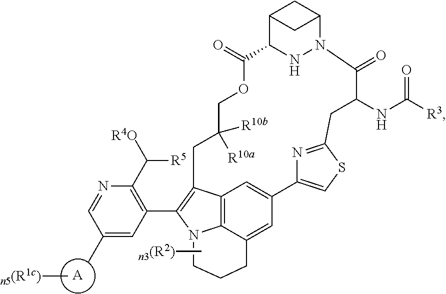

or a hydrate, solvate, N-oxide, pharmaceutically acceptable salt, stereoisomer, tautomer, or deuterated analog thereof;
Ring A is 5- to 10-membered heterocyclyl comprising 1 or 2 heteroatoms selected from N and O; and
n5 is 0, 1, 2, 3 or 4;
each of R^{1c}, R², R³, R⁴, R⁵, R^{10a}, R^{10b} and n3 is defined as above.

In one embodiment, the compound is

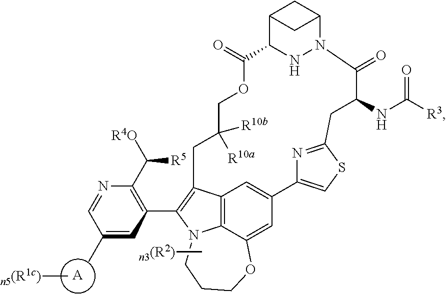

or a hydrate, solvate, N-oxide, pharmaceutically acceptable salt, stereoisomer, tautomer, or deuterated analog thereof;
Ring A is 5- to 10-membered heterocyclyl comprising 1 or 2 heteroatoms selected from N and O; and
n5 is 0, 1, 2, 3 or 4;
each of R^{1c}, R², R³, R⁴, R⁵, R^{10a}, R^{10b} and n3 is defined as above.

In one embodiment, the compound is

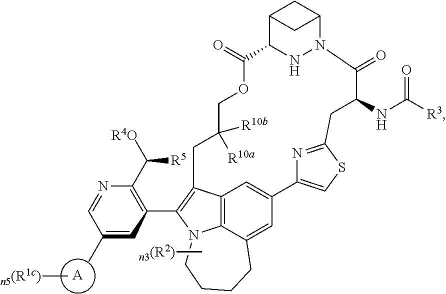

or a hydrate, solvate, N-oxide, pharmaceutically acceptable salt, stereoisomer, tautomer, or deuterated analog thereof;
Ring A is 5- to 10-membered heterocyclyl comprising 1 or 2 heteroatoms selected from N and O; and
n5 is 0, 1, 2, 3 or 4;
each of R^{1c}, R², R³, R⁴, R⁵, R^{10a}, R^{10b} and n3 is defined as above.

Aspect 11. The compound of Aspect 1 or 2, wherein the compound is formula (Xa) or (Xb)

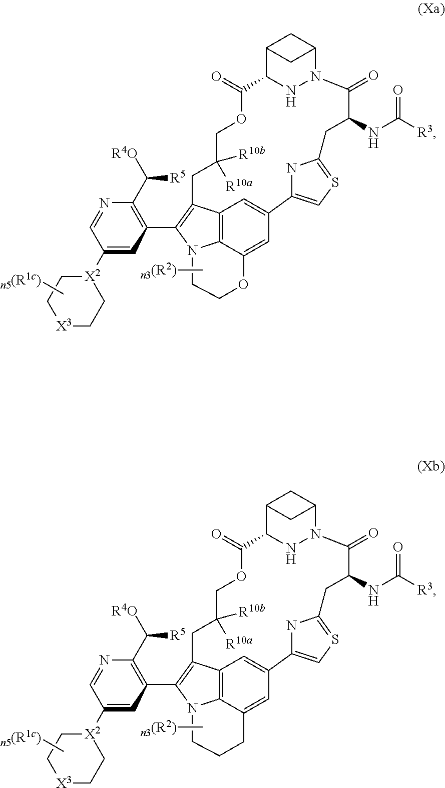

or a hydrate, solvate, N-oxide, pharmaceutically acceptable salt, stereoisomer, tautomer, or deuterated analog thereof;
X² is N or CH;
X³ is NH, CH₂ or O; and
n5 is 0, 1, 2, 3 or 4.

Aspect 12. The compound of Aspect 1 or 2, wherein the compound is formula (XIa) or (XIb):

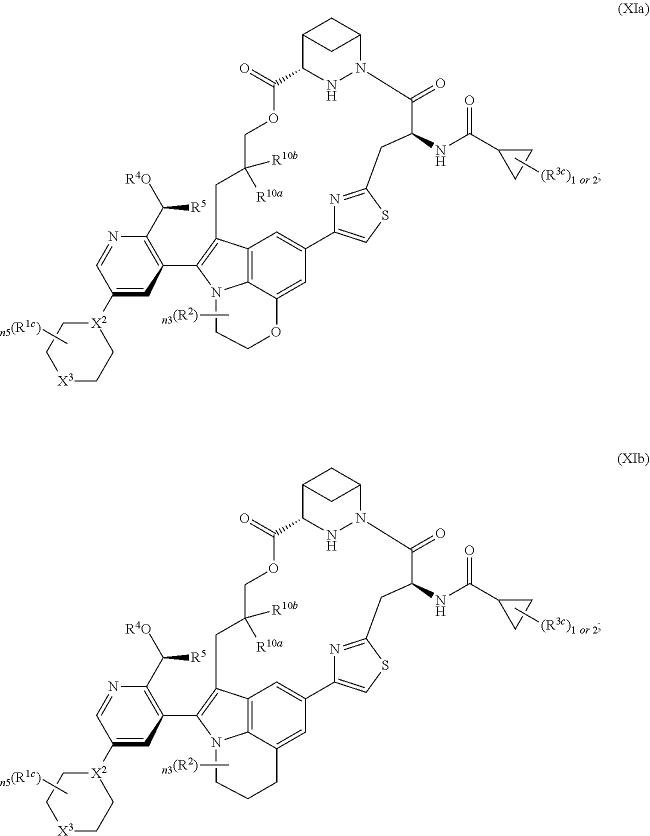

or a hydrate, solvate, N-oxide, pharmaceutically acceptable salt, stereoisomer, tautomer, or deuterated analog thereof.

In another embodiment, wherein the compound is

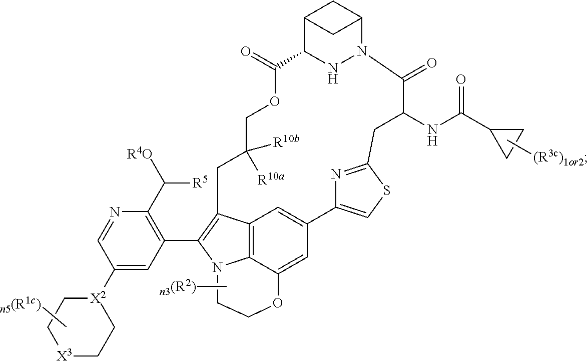

or a hydrate, solvate, N-oxide, pharmaceutically acceptable salt, stereoisomer, tautomer, or deuterated analog thereof;
each of R^{1c}, R², R³, R⁴, R⁵, R^{10a}, R^{10b}, X², X³, n5 and n3 is defined as above.

In one embodiment, wherein the compound is

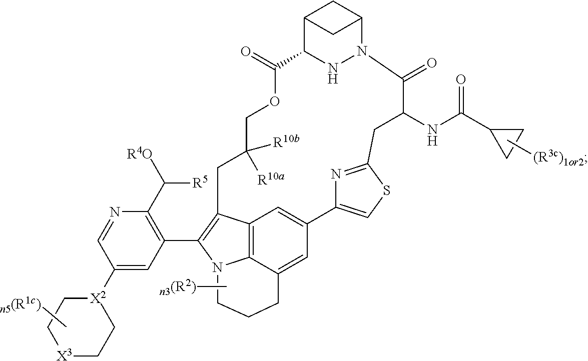

or a hydrate, solvate, N-oxide, pharmaceutically acceptable salt, stereoisomer, tautomer, or deuterated analog thereof;
each of R^{1c}, R², R^{3c}, R⁴, R⁵, R^{10a}, R^{10b}, X², X³, n5 and n3 is defined as above.

In one embodiment, wherein the compound is

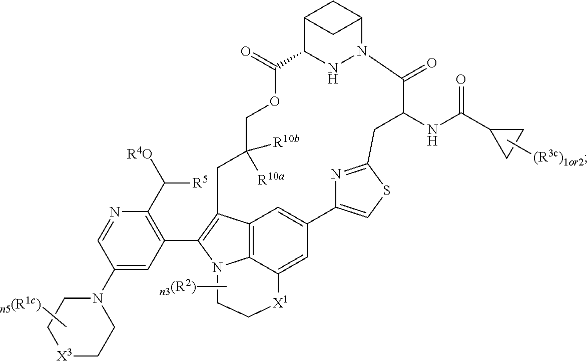

or a hydrate, solvate, N-oxide, pharmaceutically acceptable salt, stereoisomer, tautomer, or deuterated analog thereof;
each of R^{1c}, R², R^{3c}, R⁴, R⁵, R^{10a}, R^{10b}, X¹, X³, n5 and n3 is defined as above.

In one embodiment, wherein the compound is

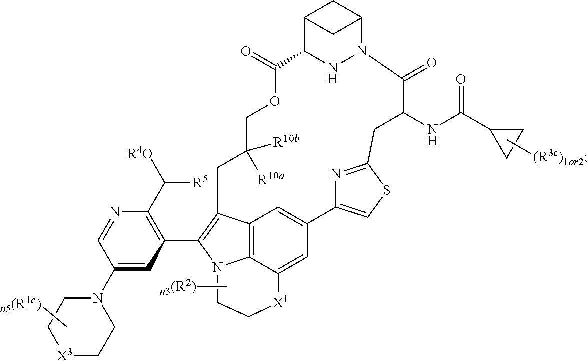

or a hydrate, solvate, N-oxide, pharmaceutically acceptable salt, stereoisomer, tautomer, or deuterated analog thereof;
each of R^{1c}, R², R^{3c}, R⁴, R⁵, R^{10a}, R^{10b}, X¹, X³, n5 and n3 is defined as above.

In a preferred embodiment, wherein the compound is

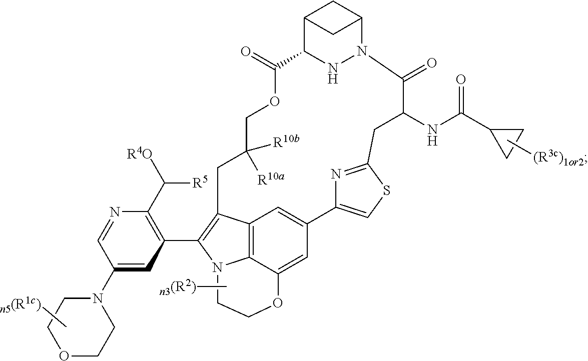

or a hydrate, solvate, N-oxide, pharmaceutically acceptable salt, stereoisomer, tautomer, or deuterated analog thereof.

In another more preferred embodiment, wherein the compound is

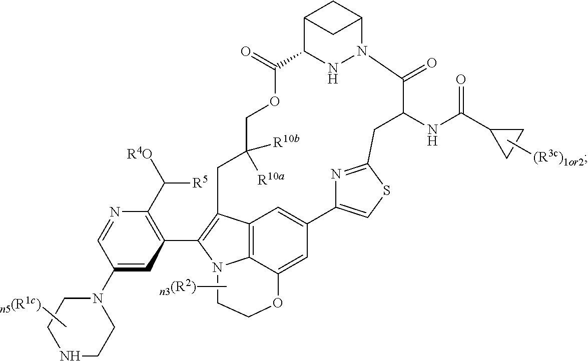

or a hydrate, solvate, N-oxide, pharmaceutically acceptable salt, stereoisomer, tautomer, or deuterated analog thereof.

In another more preferred embodiment, wherein the compound is

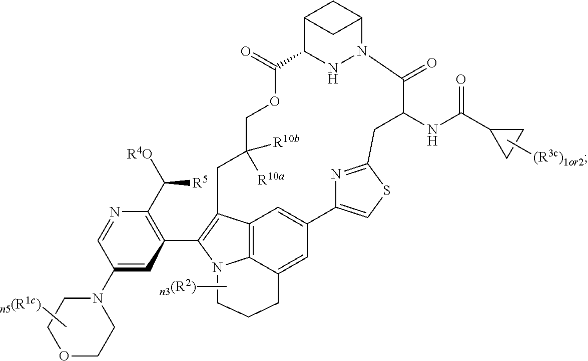

or a hydrate, solvate, N-oxide, pharmaceutically acceptable salt, stereoisomer, tautomer, or deuterated analog thereof.

In another more preferred embodiment, wherein the compound is

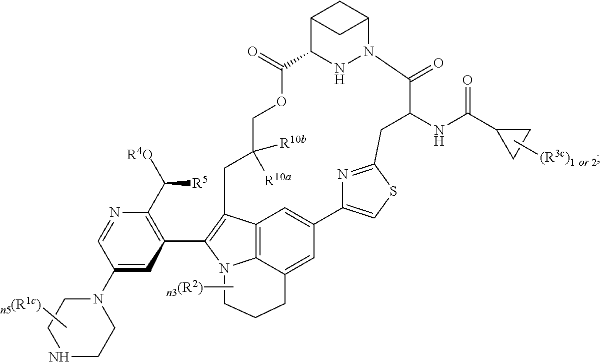

or a hydrate, solvate, N-oxide, pharmaceutically acceptable salt, stereoisomer, tautomer, or deuterated analog thereof.

In a even more preferred embodiment, wherein the compound is

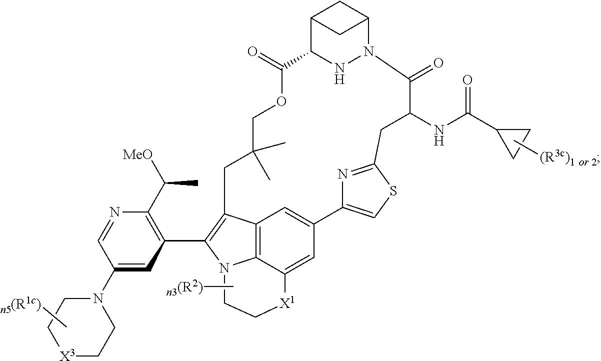

or a hydrate, solvate, N-oxide, pharmaceutically acceptable salt, stereoisomer, tautomer, or deuterated analog thereof.

Aspect 13. The compound or the hydrate, solvate, N-oxide, pharmaceutically acceptable salt, stereoisomer, tautomer, or deuterated analog of any one of Aspects 1-9, wherein X¹ is —O—.

In one embodiment, X¹ is —CH₂—.

Aspect 14. The compound or the hydrate, solvate, N-oxide, pharmaceutically acceptable salt, stereoisomer, tautomer, or deuterated analog of any one of Aspects 1-6, wherein n+n2≤3.

In one embodiment, n1+n2≤2. In one embodiment, n1+n2=2.

Aspect 15. The compound or the hydrate, solvate, N-oxide, pharmaceutically acceptable salt, stereoisomer, tautomer, or deuterated analog of Aspect 14, wherein n1 is 0 and n2 is 2.

In one embodiment, n1 is 0 and n2 is 2; and X¹ is —O—.

Aspect 16. The compound or the hydrate, solvate, N-oxide, pharmaceutically acceptable salt, stereoisomer, tautomer, or deuterated analog of any one of Aspects 1-6, wherein

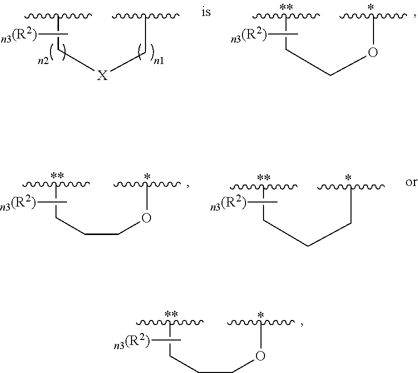

wherein * refers to the position attached to the phenyl moiety, and ** refers to the position attached to the pyrrolyl moiety.

In one embodiment,

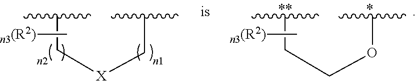

In another embodiment,

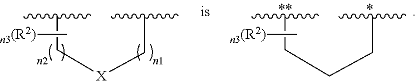

Aspect 17. The compound or the hydrate, solvate, N-oxide, pharmaceutically acceptable salt, stereoisomer, tautomer, or deuterated analog of any one of Aspects 1-8, wherein R¹ is selected from hydrogen, halogen, —C₁₋₆alkyl, —C₃₋₆cycloalkyl and 5- to 10-membered heterocyclyl, wherein each of said —C₁₋₆alkyl, —C₃₋₆cycloalkyl and 5- to 10-membered heterocyclyl is optionally substituted with at least one substituent R^{1c};
R^{1c} is each independently selected from hydrogen, halogen, —C₁₋₆alkyl, —C₂₋₆alkynyl, —C₃-C₆cycloalkyl, 3- to 6-membered heterocyclyl, —C₆-C₁₂aryl, 5- to 10-membered heteroaryl, —OR^{1e}, —COR^{1e}, —CO₂R^{1e}, —CONR^{1e}R^{1f}, —NR^{1e}R^{1f}, —NR^{1e}COR^{1f}, —NR^{1e}CO₂R^{1f}, oxo and —CN; wherein each of said —C₁₋₆alkyl, —C₂₋₆alkynyl, —C₃-C₆cycloalkyl, 3- to 6-membered heterocyclyl, —C₆-C₁₂aryl and 5- to 10-membered heteroaryl is optionally substituted with at least one substituent selected from halogen, —C₁₋₆alkoxy, —C₃-C₆cycloalkyl, 3- to 6-membered heterocyclyl, —C₆-C₁₂aryl, 5- to 12-membered heteroaryl, —CN, —OH, —NH₂ and oxo; or
when adjacent or geminal, two R^{1c} together with the atom(s) to which they are attached, form a 3- to 8-membered unsaturated or saturated ring, said ring comprising 0-3 heteroatoms independently selected from nitrogen, oxygen or sulfur and said ring is optionally substituted with at least one substituent selected from halogen, —C₁₋₆alkoxy, —C₃-C₆cycloalkyl, 3- to 6-membered heterocyclyl, —C₆-C₁₂aryl, 5- to 12-membered heteroaryl, —CN, —OH, —NH₂ and oxo;
R^{1e} and R^{1f} are each independently selected from hydrogen, —C₁₋₆alkyl, —C₃-C₆cycloalkyl, 3- to 10-membered heterocyclyl, —C₆-C₁₂aryl and 5- to 10-membered heteroaryl, wherein each of said —C₁₋₆alkyl, —C₃-C₆cycloalkyl, 3- to 10-membered heterocyclyl, —C₆-C₁₂aryl and 5- to 10-membered heteroaryl is optionally substituted with at least one substituent selected from halogen, —C₁₋₆alkoxy, —C₃-C₆cycloalkyl, 3- to 6-membered heterocyclyl, —C₆-C₁₂aryl, 5- to 12-membered heteroaryl, —CN, —OH, —NH₂ and oxo.

In a preferred embodiment, R¹ is selected from —C₃₋₆cycloalkyl and 5- to 10-membered heterocyclyl, wherein each of said —C₃₋₆cycloalkyl and 5- to 10-membered heterocyclyl is optionally substituted with at least one substituent R^{1c};
R^{1c} is each independently selected from hydrogen, halogen, —C₁₋₆alkyl, —C₃-C₆cycloalkyl, 3- to 6-membered heterocyclyl, —C₆-C₁₂aryl, 5- to 10-membered heteroaryl, —OR^{1e}, —COR^{1e}, —CO₂R^{1e}, —CONR^{1e}R^{1f}, —NR^{1e}R^{1f}, —NR^{1e}COR^{1f}, —NR^{1e}CO₂R^{1f}, oxo and —CN; wherein each of said —C₁₋₆alkyl, —C₃-C₆cycloalkyl, 3- to 6-membered heterocyclyl, —C₆-C₁₂aryl and 5- to 10-membered heteroaryl is optionally substituted with at least one substituent selected from halogen, —C₁₋₆alkoxy, —C₃-C₆cycloalkyl, 3- to 6-membered heterocyclyl, —C₆-C₁₂aryl, 5- to 12-membered heteroaryl, —CN, —OH, —NH₂ and oxo; or
when adjacent or geminal, two R^{1c} together with the atom(s) to which they are attached, form a 3- to 8-membered unsaturated or saturated ring, said ring comprising 0-3 heteroatoms independently selected from nitrogen, oxygen or sulfur and said ring is optionally substituted with at least one substituent selected from halogen, —C₁₋₆alkoxy, —C₃-C₆cycloalkyl, 3- to 6-membered heterocyclyl, —C₆-C₁₂aryl, 5- to 12-membered heteroaryl, —CN, —OH, —NH₂ and oxo;
R^{1e} and R^{1f} are each independently selected from hydrogen, —C₁₋₆alkyl, —C₃-C₆cycloalkyl, 3- to 10-membered heterocyclyl, —C₆-C₁₂aryl and 5- to 10-membered heteroaryl, wherein each of said —C₁₋₆alkyl, —C₃-C₆cycloalkyl, 3- to 10-membered heterocyclyl, —C₆-C₁₂aryl and 5- to 10-membered heteroaryl is optionally substituted with at least one substituent selected from halogen, —C₁₋₆alkoxy, —C₃-C₆cycloalkyl, 3- to 6-membered heterocyclyl, —C₆-C₁₂aryl, 5- to 12-membered heteroaryl, —CN, —OH, —NH₂ and oxo.

In another preferred embodiment, R¹ is selected from hydrogen, halogen, —C₁₋₆alkyl, —C₃₋₆cycloalkyl and 4- to 10-membered heterocyclyl, wherein each of said —C₁₋₆alkyl, —C₃₋₆cycloalkyl and 4- to 10-membered heterocyclyl is optionally substituted with at least one substituent R^{1c};
R^{1c} is each independently selected from hydrogen, halogen, —C₁₋₆alkyl, —C₃₋₆alkynyl, —C₃-C₆cycloalkyl, 3- to 6-membered heterocyclyl, —C₆-C₁₂aryl, 5- to 10-membered heteroaryl, —OR^{1e}, —COR^{1e}, —CO₂R^{1e}, —CONR^{1e}R^{1f}, —NR^{1e}R^{1f}, —NR^{1e}COR^{1f}, —NR^{1e}CO₂R^{1f}, oxo and —CN; wherein each of said —C₁₋₆alkyl, —C₂₋₆alkynyl, —C₃-C₆cycloalkyl, 3- to 6-membered heterocyclyl, —C₆-C₁₂aryl and 5- to 10-membered heteroaryl is optionally substituted with at least one substituent selected from halogen, —C₁₋₆alkoxy, —C₃-C₆cycloalkyl, 3- to 6-membered heterocyclyl, —C₆-C₁₂aryl, 5- to 12-membered heteroaryl, —CN, —OH, —NH₂ and oxo; or
when adjacent or geminal, two R^{1c} together with the atom(s) to which they are attached, form a 3- to 8-membered unsaturated or saturated ring, said ring comprising 0-3 heteroatoms independently selected from nitrogen, oxygen or sulfur and said ring is optionally substituted with at least one substituent selected from halogen, —C₁₋₆alkoxy, —C₃-C₆cycloalkyl, 3- to 6-membered heterocyclyl, —C₆-C₁₂aryl, 5- to 12-membered heteroaryl, —CN, —OH, —NH₂ and oxo; or
two non-adjacent and non-geminal R^{1c} form a bridge, said bridge is constituted with 1-3 —CH₂— and said —CH₂— is optionally substituted with at least one substituent selected from halogen, —C₁₋₆alkoxy, —C₃-C₆cycloalkyl, 3- to 6-membered heterocyclyl, —C₆-C₁₂aryl, 5- to 12-membered heteroaryl, —CN, —OH, —NH₂ and oxo;
R^{1e} and R^{1f} are each independently selected from hydrogen, —C₁₋₆alkyl, —C₃-C₆cycloalkyl, 3- to 10-membered heterocyclyl, —C₆-C₁₂aryl and 5- to 10-membered heteroaryl, wherein each of said —C₁₋₆alkyl, —C₃-C₆cycloalkyl, 3- to 10-membered heterocyclyl, —C₆-C₁₂aryl and 5- to 10-membered heteroaryl is optionally substituted with at least one substituent selected from halogen, —C₁₋₆alkoxy, —C₃-C₆cycloalkyl, 3- to 6-membered heterocyclyl, —C₆-C₁₂aryl, 5- to 12-membered heteroaryl, —CN, —OH, —NH₂ and oxo.

Aspect 18. The compound or the hydrate, solvate, N-oxide, pharmaceutically acceptable salt, stereoisomer, tautomer, or deuterated analog of Aspect 17, wherein R¹ is selected from —C₃₋₆cycloalkyl and 5- to 10-membered heterocyclyl, wherein each of said —C₃₋₆cycloalkyl and 5- to 10-membered heterocyclyl is optionally substituted with at least one substituent R^{1c};
R^{1c} is each independently selected from hydrogen, halogen, —C₁₋₃alkyl, —C₃-C₆cycloalkyl, —OR^{1e}, —NR^{1e}R^{1f}, oxo and —CN;
R^{1e} and R^{1f} are each independently selected from hydrogen, —C₁₋₃alkyl and —C₃-C₆cycloalkyl.

In a preferred embodiment, R¹ is selected from —C₃₋₆cycloalkyl and 5- to 10-membered heterocyclyl, wherein each of said —C₃₋₆cycloalkyl and 5- to 10-membered heterocyclyl is optionally substituted with at least one substituent R^{1c};
R^{1c} is each independently selected from hydrogen, halogen, —C₁₋₃alkyl, —C₃-C₆cycloalkyl, —C₂₋₄alkynyl, —OR^{1e}, —NR^{1e}R^{1f}, oxo and —CN;
R^{1e} and R^{1f} are each independently selected from hydrogen, —C₁₋₃alkyl and —C₃-C₆cycloalkyl.

Aspect 19. The compound or the hydrate, solvate, N-oxide, pharmaceutically acceptable salt, stereoisomer, tautomer, or deuterated analog of any one of Aspects 1-8, wherein R¹ is

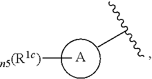

wherein Ring A is 5- to 6-membered hetero monocyclic ring comprising 1 or 2 heteroatoms atoms selected from N and O or 8- to 10-membered hetero bicyclic ring comprising 2 or 3 heteroatoms atoms selected from N and O;
R^{1c} is each independently selected from hydrogen, halogen, —C₁₋₃alkyl, —C₃-C₆cycloalkyl, —OR^{1e}, —NR^{1e}R^{1f}, oxo and —CN; each of said —C₁₋₃alkyl and —C₃-C₆cycloalkyl is optionally substituted with at least one substituent selected from halogen, —C₁₋₃alkoxy, —C₃-C₆cycloalkyl, —CN, —OH, —NH₂ and oxo;
R^{1e} and R^{1f} are each independently selected from hydrogen, —C₁₋₃alkyl and —C₃-C₆cycloalkyl; or
when adjacent or geminal, two R^{1c} together with the atom(s) to which they are attached, form a 3- to 8-membered unsaturated or saturated ring, said ring comprising 0-3 heteroatoms independently selected from nitrogen, oxygen or sulfur and said ring is optionally substituted with at least one substituent selected from halogen, —C₁₋₃alkoxy, —C₃-C₆cycloalkyl, —CN, —OH, —NH₂ and oxo;
n5 is 0, 1, 2, 3 or 4.

In a preferred embodiment, R¹ is

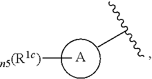

wherein Ring A is 5- to 6-membered hetero monocyclic ring comprising 1 or 2 heteroatoms atoms selected from N and O or 8- to 10-membered hetero bicyclic ring comprising 2 or 3 heteroatoms atoms selected from N and O;
R^{1c} is each independently selected from hydrogen, halogen, —C₁₋₃alkyl, —C₃-C₆cycloalkyl, —C₂₋₆alkynyl —OR^{1e}, —NR^{1e}R^{1f}, oxo and —CN; each of said —C₁₋₃alkyl, —C₃-C₆cycloalkyl and —C₂₋₆alkynyl is optionally substituted with at least one substituent selected from halogen, —C₁₋₃alkoxy, —C₃-C₆cycloalkyl, —CN, —OH, —NH₂ and oxo;
R^{1e} and R^{1f} are each independently selected from hydrogen, —C₁₋₃alkyl and —C₃-C₆cycloalkyl; or
when adjacent or geminal, two R^{1c} together with the atom(s) to which they are attached, form a 3- to 8-membered unsaturated or saturated ring, said ring comprising 0-3 heteroatoms independently selected from nitrogen, oxygen or sulfur and said ring is optionally substituted with at least one substituent selected from halogen, —C₁₋₃alkoxy, —C₃-C₆cycloalkyl, —CN, —OH, —NH₂ and oxo;
n5 is 0, 1, 2, 3 or 4.

In a more embodiment, R¹ is

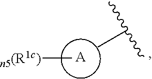

wherein Ring A is 4-, 5- or 6-membered hetero monocyclic ring comprising 1 or 2 heteroatoms atoms selected from N and O or 8- to 10-membered hetero bicyclic ring comprising 2 or 3 heteroatoms atoms selected from N and O;
R^{1c} is each independently selected from hydrogen, halogen, —C₁₋₃alkyl, —C₂₋₆alkynyl, —C₃-C₆cycloalkyl, 4- to 6-membered hetero monocyclic ring, —OR^{1e}, —NR^{1e}R^{1f}, oxo and —CN; each of said —C₁₋₃alkyl, —C₂₋₆alkynyl, —C₃-C₆cycloalkyl and 4- to 6-membered hetero monocyclic ring is optionally substituted with at least one substituent selected from halogen, —C₁₋₃alkoxy, —C₃-C₆cycloalkyl, —CN, —OH, —NH₂ and oxo;
R^{1e} and R^{1f} are each independently selected from hydrogen, —C₁₋₃alkyl and —C₃-C₆cycloalkyl; or
when adjacent or geminal, two R^{1c} together with the atom(s) to which they are attached, form a 3- to 8-membered unsaturated or saturated ring, said ring comprising 0-3 heteroatoms independently selected from nitrogen, oxygen or sulfur and said ring is optionally substituted with at least one substituent selected from halogen, —C₁₋₃alkoxy, —C₃-C₆cycloalkyl, —CN, —OH, —NH₂ and oxo; or
two non-adjacent and non-geminal RIC form a bridge, said bridge is constituted with 1-3 —CH₂— and said —CH₂— is optionally substituted with at least one substituent selected from halogen, —C₁₋₃alkoxy, —C₃-C₆cycloalkyl, —CN, —OH, —NH₂ and oxo;
n5 is 0, 1, 2, 3 or 4.

Aspect 20. The compound or the hydrate, solvate, N-oxide, pharmaceutically acceptable salt, stereoisomer, tautomer, or deuterated analog of Aspect 19, wherein R¹ is

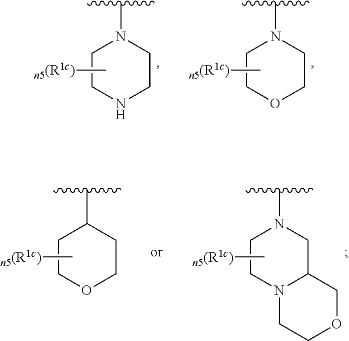

n5 is 0, 1, 2, 3 or 4;
R^{1c} is methyl, ethyl, propyl or cyclopropyl, wherein each of said methyl, ethyl, propyl and cyclopropyl is optionally substituted with at least one substituent selected from halogen, —C₁₋₃alkoxy, —C₃-C₆cycloalkyl, —CN, —OH, —NH₂ and oxo; or
two geminal R^{1c} together with the atom to which they are attached, form a 3- to 6-membered unsaturated or saturated ring, said ring comprising 0-3 heteroatoms independently selected from nitrogen, oxygen and sulfur and said ring is optionally substituted with at least one substituent selected from halogen, —C₁₋₃alkoxy, —C₃-C₆cycloalkyl, —CN, —OH, —NH₂ and oxo.

In another embodiment, R¹ is

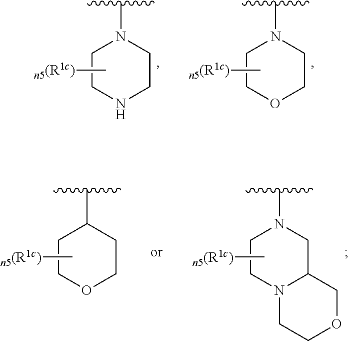

n5 is 0, 1, 2, 3 or 4;
R^{1c} is methyl, ethyl, propyl, cyclopropyl or —C₂₋₄alkynyl, wherein each of said methyl, ethyl, propyl, cyclopropyl and —C₂₋₄alkynyl is optionally substituted with at least one substituent selected from halogen, —C₁₋₃alkoxy, —C₃-C₆cycloalkyl, —CN, —OH, —NH₂ and oxo; or
two geminal R^{1c} together with the atom to which they are attached, form a 3- to 6-membered unsaturated or saturated ring, said ring comprising 0-3 heteroatoms independently selected from nitrogen, oxygen and sulfur and said ring is optionally substituted with at least one substituent selected from halogen, —C₁₋₃alkoxy, —C₃-C₆cycloalkyl, —CN, —OH, —NH₂ and oxo.

In another embodiment, R¹ is

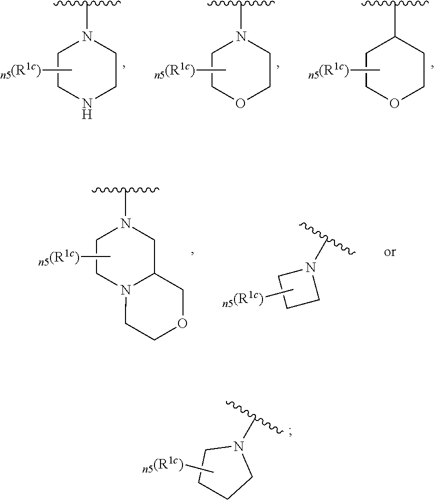

n5 is 0, 1, 2, 3 or 4;
R^{1c} is methyl, ethyl, propyl, cyclopropyl, cyclobutyl, oxetanyl or —C₂₋₄alkynyl, wherein each of said methyl, ethyl, propyl, cyclopropyl and —C₂₋₄alkynyl is optionally substituted with at least one substituent selected from halogen, —C₁₋₃alkoxy, —C₃-C₆cycloalkyl, —CN, —OH, —NH₂ and oxo; or
two geminal R^{1c} together with the atom to which they are attached, form a 3- to 6-membered unsaturated or saturated ring, said ring comprising 0-3 heteroatoms independently selected from nitrogen, oxygen and sulfur and said ring is optionally substituted with at least one substituent selected from halogen, —C₁₋₃alkoxy, —C₃-C₆cycloalkyl, —CN, —OH, —NH₂ and oxo; or
two adjacent R^{1c} together with the atoms to which they are attached, form a 3- to 6-membered unsaturated or saturated ring, said ring comprising 0-3 heteroatoms independently selected from nitrogen, oxygen and sulfur and said ring is optionally substituted with at least one substituent selected from halogen, —C₁₋₃alkoxy, —C₃-C₆cycloalkyl, —CN, —OH, —NH₂ and oxo; or
two non-adjacent and non-geminal R^{1c} form a bridge, said bridge is constituted with 1-3 —CH₂— and said —CH₂— is optionally substituted with at least one substituent selected from halogen, —C₁₋₃alkoxy, —C₃-C₆cycloalkyl, —CN, —OH, —NH₂ and oxo.

In another embodiment, R^{1c} is methyl, ethyl or propyl. In another embodiment, R^{1c} is methyl or ethyl. In another embodiment, R^{1c} is methyl. In another embodiment, two geminal R^{1c} together with the atom to which they are attached, form a 3- to 6-membered unsaturated or saturated ring. In another embodiment, two geminal R^{1c} together with the atom to which they are attached, form a 3-membered unsaturated or saturated ring. In another embodiment, two geminal R^{1c} together with the atom to which they are attached, form a 4-membered unsaturated or saturated ring. In another embodiment, two geminal R^{1c} together with the atom to which they are attached, form a 5-membered unsaturated or saturated ring. In another embodiment, two geminal R^{1c} together with the atom to which they are attached, form a 3-membered saturated ring. In another embodiment, two geminal R^{1c} together with the atom to which they are attached, form a 4-membered saturated ring. In another embodiment, two geminal RIC together with the atom to which they are attached, form a 5-membered saturated ring.

In one embodiment, R¹ is

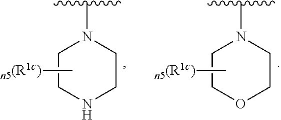

In a preferred embodiment, R¹ is

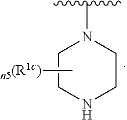

In another preferred embodiment, R¹ is

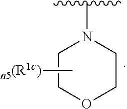

In another preferred embodiment, R¹ is

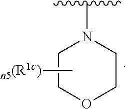

Aspect 21. The compound or the hydrate, solvate, N-oxide, pharmaceutically acceptable salt, stereoisomer, tautomer, or deuterated analog of Aspect 20, wherein R¹ is

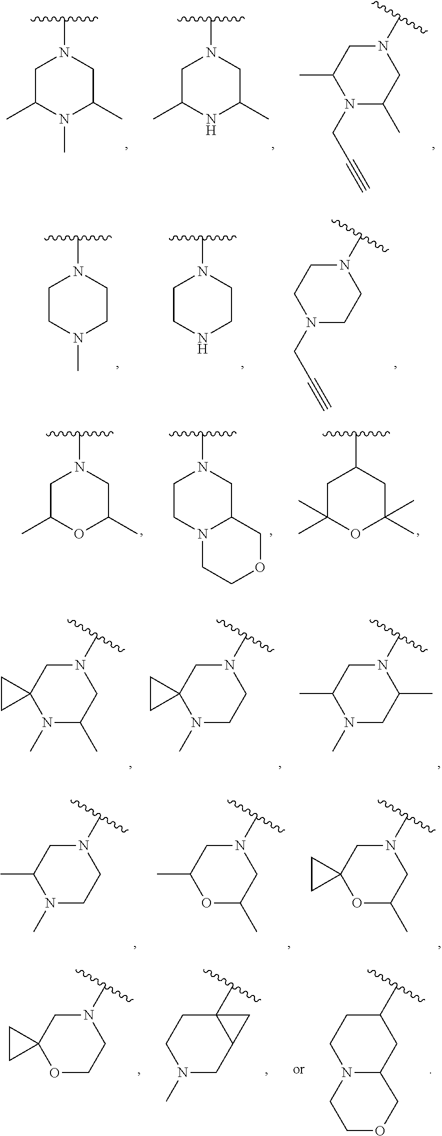

In a preferred embodiment, R¹ is

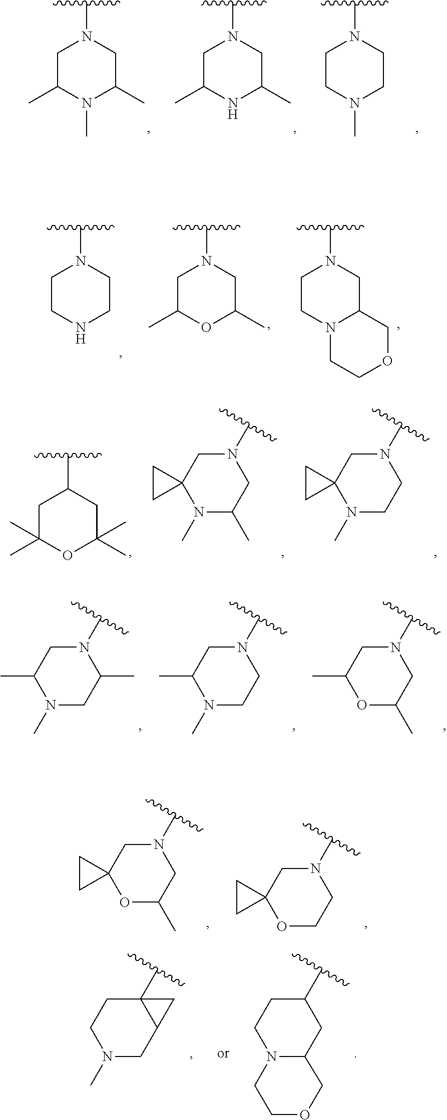

In another preferred embodiment, R¹ is

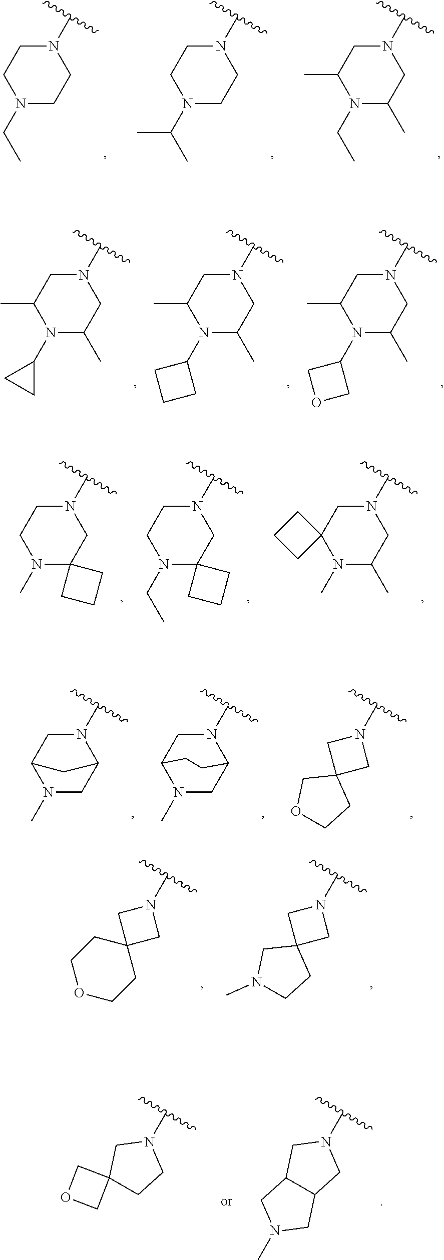

Aspect 22. The compound of Aspect 20, wherein R¹ is

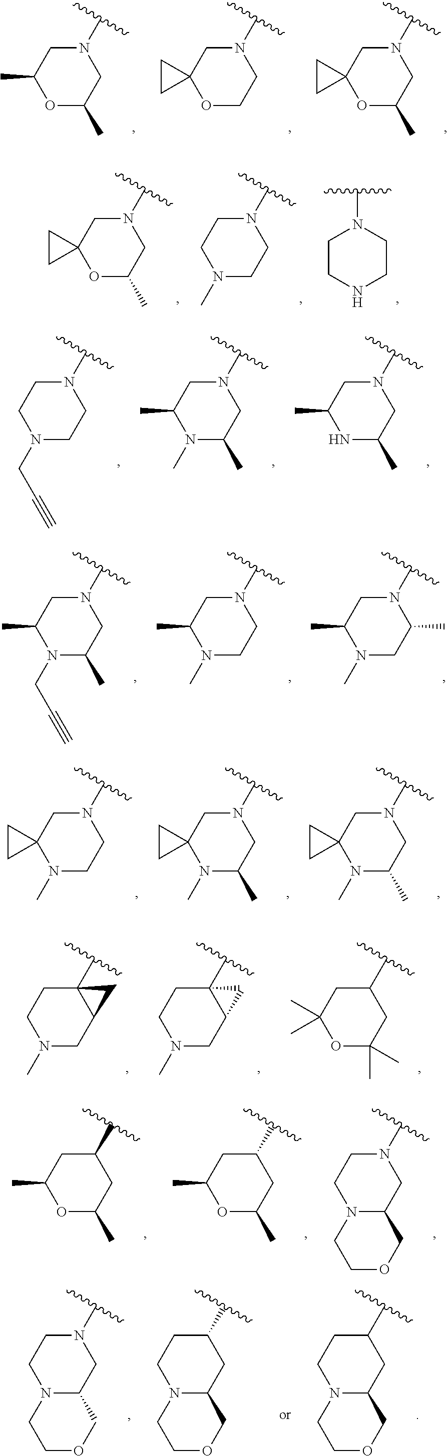

In a preferred embodiment, R¹ is

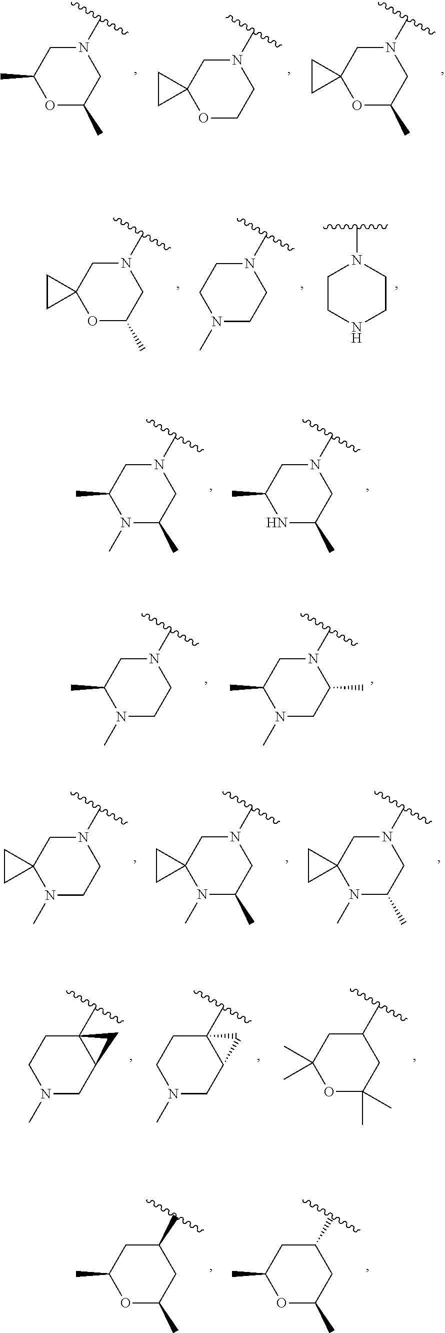

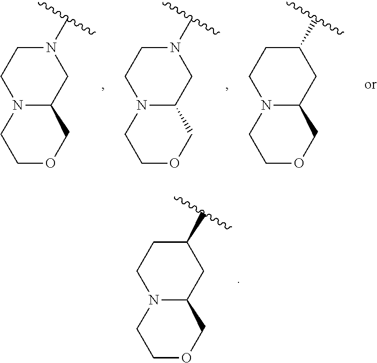 [SMILES: *N1CCN2CCOC[C@@H]2C1 ; *N1CCN2CCOC[C@H]2C1 ; *[C@H]1CCN2CCOC[C@@H]2C1 ; *[C@@H]1CCN2CCOC[C@@H]2C1]

In another preferred embodiment, R¹ is

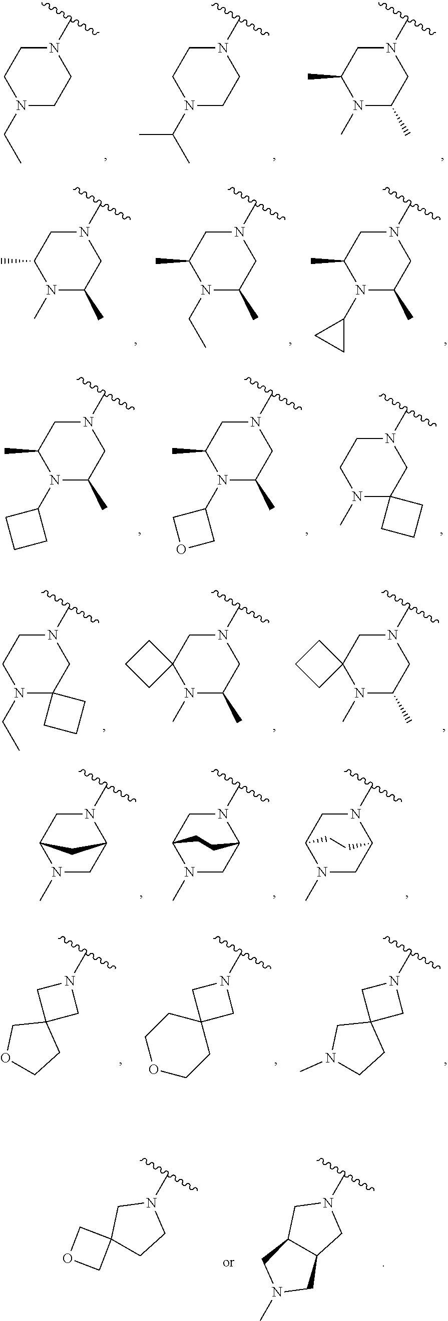

Aspect 23. The compound or the hydrate, solvate, N-oxide, pharmaceutically acceptable salt, stereoisomer, tautomer, or deuterated analog of any one of Aspects 1-12, wherein R² is each independently selected from hydrogen, halogen, —C₁₋₆alkyl, —C₃₋₆cycloalkyl and 3- to 6-membered heterocyclyl, wherein each of said —C₁₋₆alkyl, —C₃₋₆cycloalkyl and 3- to 6-membered heterocyclyl is optionally substituted with at least one substituent R^{2c};
R^{2c} is each independently selected from hydrogen, halogen, —C₁₋₃alkyl, —C₃-C₆cycloalkyl, 3- to 6-membered heterocyclyl, —OR^{2d}, —COR^{2d}, —CO₂R^{2d}, —CONR^{2d}R^{2e}, —NR^{2d}R^{2e}, —NR^{2d}COR^{2e}, —NR^{2d}CO₂R^{2e}, oxo and —CN;
R^{2d} and R^{2e} are each independently selected from hydrogen, —C₁₋₃alkyl and —C₃-C₆cycloalkyl, wherein each of said —C₁₋₃alkyl and —C₃-C₆cycloalkyl is optionally substituted with at least one substituent selected from halogen, —C₁₋₃alkoxy, —C₃₋₆cycloalkyl, 3- to 6-membered heterocyclyl, —CN, —OH, —NH₂ and oxo.

Aspect 24. The compound or the hydrate, solvate, N-oxide, pharmaceutically acceptable salt, stereoisomer, tautomer, or deuterated analog of Aspect 23, wherein R² is each independently selected from hydrogen, methyl, ethyl and propyl.

In one embodiment, (i) R² is each independently selected from hydrogen, methyl, ethyl and propyl; and n3 is 0, 1 or 2; In another embodiment, (ii) two geminal R² together with the atom to which they are attached, form a 3-, 4- or 5-membered carbon ring. In one embodiment, R² is hydrogen. In one embodiment, R² is methyl. In one embodiment, R² is ethyl.

Aspect 25. The compound or the hydrate, solvate, N-oxide, pharmaceutically acceptable salt, stereoisomer, tautomer, or deuterated analog of any one of Aspects 23-24, wherein R² is methyl; and
n3 is 0, 1 or 2.

Aspect 26. The compound or the hydrate, solvate, N-oxide, pharmaceutically acceptable salt, stereoisomer, tautomer, or deuterated analog of any one of Aspects 1-12, wherein two geminal R² together with the atom to which they are attached, form a 3- to 5-membered ring, said ring comprising 0-3 heteroatoms independently selected from nitrogen, oxygen and sulfur, said ring is optionally substituted with at least one substituent R²;
R^{2c} is each independently selected from hydrogen, halogen, —C₁₋₃alkyl, —C₃-C₆cycloalkyl, 3- to 6-membered heterocyclyl, —OR^{2d}, —COR^{2d}, —CO₂R^{2d}, —CONR^{2d}R^{2e}, —NR^{2d}R^{2e}, —NR^{2d}COR^{2e}, —NR^{2d}CO₂R^{2e}, oxo and —CN;
R^{2d} and R^{2e} are each independently selected from hydrogen, —C₁₋₃alkyl and —C₃-C₆cycloalkyl, wherein each of said —C₁₋₃alkyl and —C₃-C₆cycloalkyl is optionally substituted with at least one substituent selected from halogen, —C₁₋₃alkoxy, —C₃-C₆cycloalkyl, 3- to 6-membered heterocyclyl, —CN, —OH, —NH₂ and oxo.

Aspect 27. The compound or the hydrate, solvate, N-oxide, pharmaceutically acceptable salt, stereoisomer, tautomer, or deuterated analog of Aspect 26, wherein two geminal R² together with the atom to which they are attached, form a 3-, 4- or 5-membered carbon ring.

Aspect 28. The compound or the hydrate, solvate, N-oxide, pharmaceutically acceptable salt, stereoisomer, tautomer, or deuterated analog of any one of Aspects 1-6, wherein

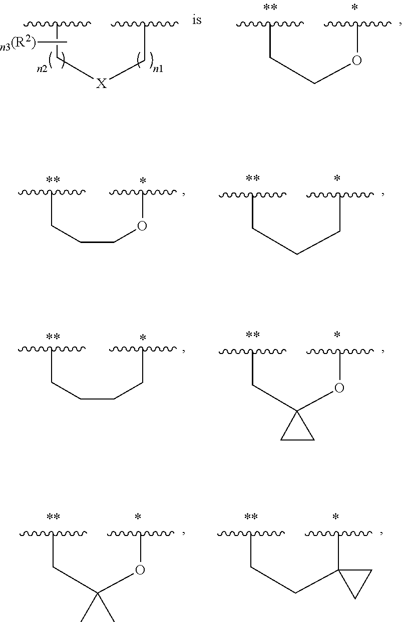

wherein * refers to the position attached to the phenyl moiety, and ** refers to the position attached to the pyrrolyl moiety.

Aspect 29. The compound or the hydrate, solvate, N-oxide, pharmaceutically acceptable salt, stereoisomer, tautomer, or deuterated analog of any one of Aspects 1-11, wherein R³ is selected from —C₁₋₆alkyl, —C₃₋₆cycloalkyl, and 3- to 6-membered heterocyclyl, wherein each of said —C₁₋₆alkyl, —C₃-C₆cycloalkyl and 3- to 6-membered heterocyclyl is optionally substituted with at least one substituent R^{3c};
R^{3c} is selected from hydrogen, halogen, —C₁₋₃alkyl, —C₃-C₆cycloalkyl, —OR^{3e}, oxo and —CN; wherein each of said —C₁₋₃alkyl and —C₃-C₆cycloalkyl is optionally substituted with at least one substituent selected from halogen, —C₁₋₃alkoxy, —C₃-C₆cycloalkyl, —CN, —OH, —NH₂ and oxo;
R^{3e} is selected from hydrogen, —C₁₋₃alkyl, —C₃-C₆cycloalkyl and 3- to 6-membered heterocyclyl, wherein each of said —C₁₋₃alkyl, —C₃-C₆cycloalkyl and 3- to 6-membered heterocyclyl is optionally substituted with at least one substituent selected from halogen, —C₁₋₃alkoxy, —C₃-C₆cycloalkyl, —CN, —OH, —NH₂ and oxo.

Aspect 30. The compound or the hydrate, solvate, N-oxide, pharmaceutically acceptable salt, stereoisomer, tautomer, or deuterated analog of Aspect 29, wherein R³ is selected from —C₁₋₃alkyl and —C₃₋₅cycloalkyl, wherein each of said —C₁₋₃alkyl and —C₃-C₅cycloalkyl is optionally substituted with at least one substituent R^{3c};
R^{3c} is selected from hydrogen, halogen, —C₁₋₃alkyl, —C₃-C₆cycloalkyl, —C₁₋₃alkoxy, oxo and —CN.

Aspect 31. The compound or the hydrate, solvate, N-oxide, pharmaceutically acceptable salt, stereoisomer, tautomer, or deuterated analog of Aspect 30, wherein R³ is

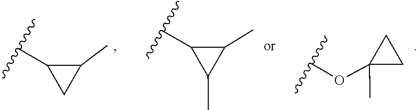

In one embodiment, R³ is —CF₃,

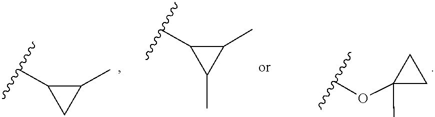

Aspect 32. The compound or the hydrate, solvate, N-oxide, pharmaceutically acceptable salt, stereoisomer, tautomer, or deuterated analog of Aspect 30, wherein R³ is

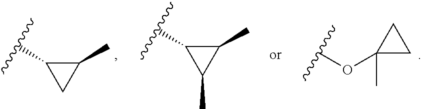

In a preferred embodiment, R³ is

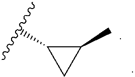 [SMILES: *[C@H]1C[C@@H]1C]

In another preferred embodiment, R³ is

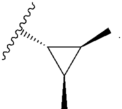 [SMILES: *[C@@H]1[C@@H](C)[C@H]1C]

In another preferred embodiment, R³ is

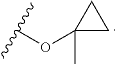 [SMILES: *OC1(C)CC1]

Aspect 33. The compound or the hydrate, solvate, N-oxide, pharmaceutically acceptable salt, stereoisomer, tautomer, or deuterated analog of any one of Aspects 1-12, wherein R⁴ is selected from —C₁₋₃ alkyl, —C₃₋₆cycloalkyl and 3- to 6-membered heterocyclyl, wherein each of said —C₁₋₃alkyl, —C₃₋₆cycloalkyl and 3- to 6-membered heterocyclyl is optionally substituted with at least one substituent R^{4a};
R^{4a} is independently selected from hydrogen, halogen, —C₁₋₃alkyl, —C₃-C₆cycloalkyl, —OR^{4b}, NR^{4b}R^{4c}, oxo and —CN;
R^{4b} and R^{4c} are each independently selected from hydrogen, —C₁₋₆alkyl and —C₃-C₆cycloalkyl.

In one embodiment, R⁴ is selected from —C₁₋₃alkyl, —C₃₋₆cycloalkyl and 3- to 6-membered heterocyclyl, wherein each of said —C₁₋₃alkyl, —C₃₋₆cycloalkyl and 3- to 6-membered heterocyclyl is optionally substituted with at least one substituent R^{4a};
R^{4a} is independently selected from hydrogen, halogen, —C₁₋₃alkyl, —C₃-C₆cycloalkyl, —OR^{4b}, NR^{4b}R^{4c}, oxo and —CN;
R^{4b} and R^{4c} are each independently selected from hydrogen, —C₁₋₆alkyl and —C₃-C₆cycloalkyl; and/or
R⁵ is selected from hydrogen, halogen, —C₁₋₃alkyl and —C₃₋₆cycloalkyl, wherein each of said —C₁₋₃alkyl and —C₃₋₆cycloalkyl is optionally substituted with at least one substituent R^{5a};
R^{5a} is independently selected from hydrogen, halogen, —C₁₋₃alkyl, —C₃-C₆cycloalkyl, 3- to 6-membered heterocyclyl, —OR^{5b}, —NR^{5b}R^{5c}, oxo and —CN;
R^{5b} and R^{5c} are each independently selected from hydrogen, —C₁₋₆alkyl and —C₃-C₆cycloalkyl, wherein each of said —C₁₋₆alkyl and —C₃-C₆cycloalkyl is optionally substituted with at least one substituent selected from halogen, —C₁₋₆alkoxy, —C₃-C₆cycloalkyl, 3- to 6-membered heterocyclyl, —C₆-C₁₂aryl, 5- to 12-membered heteroaryl, —CN, —OH, —NH₂ and oxo.

Aspect 34. The compound or the hydrate, solvate, N-oxide, pharmaceutically acceptable salt, stereoisomer, tautomer, or deuterated analog of Aspect 33, wherein R⁴ is selected from —C₁₋₃alkyl and —C₃₋₆cycloalkyl.

In one embodiment, R⁴ is selected from —C₁₋₃alkyl and —C₃₋₆cycloalkyl; and/or R⁵ is selected from hydrogen, halogen, —C₁₋₃alkyl and —C₃₋₆cycloalkyl.

Aspect 35. The compound or the hydrate, solvate, N-oxide, pharmaceutically acceptable salt, stereoisomer, tautomer, or deuterated analog of Aspect 34, wherein R⁴ is selected from methyl, ethyl and propyl. In a preferred embodiment, R⁴ is methyl. In another preferred embodiment, R⁴ is ethyl. In another preferred embodiment, R⁴ is propyl.

In one embodiment, R⁴ is selected from methyl, ethyl and propyl; and/or R⁵ is selected from methyl, ethyl and propyl. In a preferred embodiment, R⁴ is methyl; and/or R⁵ is methyl.

Aspect 36. The compound or the hydrate, solvate, N-oxide, pharmaceutically acceptable salt, stereoisomer, tautomer, or deuterated analog of any one of Aspects 1-12, wherein R⁵ is selected from hydrogen, halogen, —C₁₋₃alkyl and —C₃₋₆cycloalkyl, wherein each of said —C₁₋₃alkyl and —C₃₋₆cycloalkyl is optionally substituted with at least one substituent R^{5a};
R^{5a} is independently selected from hydrogen, halogen, —C₁₋₃alkyl, —C₃-C₆cycloalkyl, 3- to 6-membered heterocyclyl, —OR^{5b}, —NR^{5b}R^{5c}, oxo and —CN;
R^{5b} and R^{5c} are each independently selected from hydrogen, —C₁₋₆alkyl and —C₃-C₆cycloalkyl, wherein each of said —C₁₋₆alkyl and —C₃-C₆cycloalkyl is optionally substituted with at least one substituent selected from halogen, —C₁₋₆alkoxy, —C₃-C₆cycloalkyl, 3- to 6-membered heterocyclyl, —C₆-C₁₂aryl, 5- to 12-membered heteroaryl, —CN, —OH, —NH₂ and oxo.

Aspect 37. The compound or the hydrate, solvate, N-oxide, pharmaceutically acceptable salt, stereoisomer, tautomer, or deuterated analog of Aspect 36, wherein R⁵ is selected from hydrogen, halogen, —C₁₋₃alkyl and —C₃₋₆cycloalkyl.

Aspect 38. The compound or the hydrate, solvate, N-oxide, pharmaceutically acceptable salt, stereoisomer, tautomer, or deuterated analog of Aspect 37, wherein R⁵ is selected from methyl, ethyl and propyl.

In a preferred embodiment, R⁵ is methyl. In another preferred embodiment, R⁵ is ethyl. In another preferred embodiment, R⁵ is propyl.

Aspect 39. The compound or the hydrate, solvate, N-oxide, pharmaceutically acceptable salt, stereoisomer, tautomer, or deuterated analog of any one of Aspects 1-12 and 33-38, wherein R⁴ is methyl and R⁵ is methyl.

Aspect 40. The compound or the hydrate, solvate, N-oxide, pharmaceutically acceptable salt, stereoisomer, tautomer, or deuterated analog of Aspect 1, wherein R⁶ is selected from hydrogen and —C₁₋₃alkyl.

Aspect 41. The compound or the hydrate, solvate, N-oxide, pharmaceutically acceptable salt, stereoisomer, tautomer, or deuterated analog of Aspect 40, wherein R⁶ is hydrogen.

Aspect 42. The compound or the hydrate, solvate, N-oxide, pharmaceutically acceptable salt, stereoisomer, tautomer, or deuterated analog of any one of Aspects 1-9, wherein R⁷, R⁸ and R⁹ are each independently selected from hydrogen, halogen, —C₁₋₆alkyl and —CN.

Aspect 43. The compound or the hydrate, solvate, N-oxide, pharmaceutically acceptable salt, stereoisomer, tautomer, or deuterated analog of Aspect 42, wherein R⁷, R⁸ and R⁹ are each hydrogen.

Aspect 44. The compound or the hydrate, solvate, N-oxide, pharmaceutically acceptable salt, stereoisomer, tautomer, or deuterated analog of any one of Aspects 1-12, wherein R^{10a} and R^{10b} are each independently selected from hydrogen and —C₁₋₃alkyl; or
R^{10a} and R^{10b} together with the atom to which they are attached, form a cyclopropyl, cyclobutyl or cyclopentyl.

Aspect 45. The compound or the hydrate, solvate, N-oxide, pharmaceutically acceptable salt, stereoisomer, tautomer, or deuterated analog of Aspect 44, wherein at least one of R^{10a} and R^{10b} is —C₁₋₃alkyl. In one embodiment, R^{10a} and R^{10b} are each independently methyl, ethyl or propyl. In a preferred embodiment, R^{10a} and R^{10b} are each independently methyl or ethyl.

Aspect 46. The compound or the hydrate, solvate, N-oxide, pharmaceutically acceptable salt, stereoisomer, tautomer, or deuterated analog of Aspect 45, wherein both of R^{10a} and R^{10b} are methyl.

In one embodiment, both of R^{10a} and R^{10b} are methyl; or R^{10a} and R^{10b} together with the atom to which they are attached, form a cyclopropyl.

Aspect 47. The compound or the hydrate, solvate, N-oxide, pharmaceutically acceptable salt, stereoisomer, tautomer, or deuterated analog of Aspect 44, wherein R^{10a} and R^{10b} together with the atom to which they are attached, form a cyclopropyl.

Aspect 48. The compound or the hydrate, solvate, N-oxide, pharmaceutically acceptable salt, stereoisomer, tautomer, or deuterated analog of any one of Aspects 1-5, wherein R^{11a}, R^{11b}, R^{12a}, R^{12b}, R^{13a} and R^{13b} are each independently selected from hydrogen, halogen and —C₁₋₃alkyl.

In a preferred embodiment, R^{11a}, R^{11b}, R^{12a}, R^{12b}, R^{13a} and R^{13b} are each hydrogen.

Aspect 49. The compound or the hydrate, solvate, N-oxide, pharmaceutically acceptable salt, stereoisomer, tautomer, or deuterated analog of Aspect 48, wherein R^{11a}, R^{11b}, R^{12a}, R^{12b}, R^{13a} and R^{13b} are each hydrogen.

Aspect 50. The compound or the hydrate, solvate, N-oxide, pharmaceutically acceptable salt, stereoisomer, tautomer, or deuterated analog of any one of Aspects 1-5, wherein the

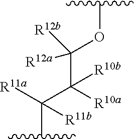

moiety is

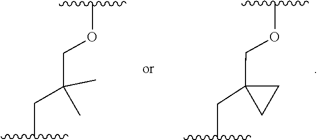

Aspect 51. A compound, or the hydrate, solvate, an N-oxide, pharmaceutically acceptable salt, stereoisomer, tautomer, or deuterated analog thereof, wherein the compound is selected from Table 1:

TABLE 1

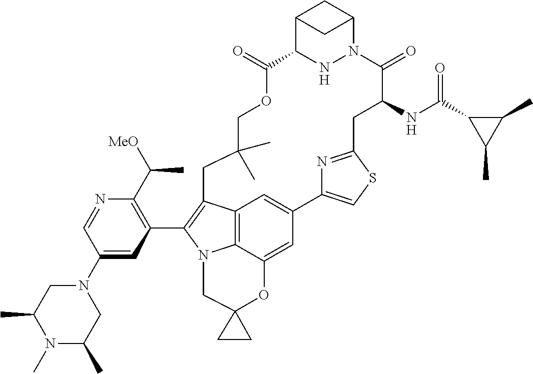

Example 1

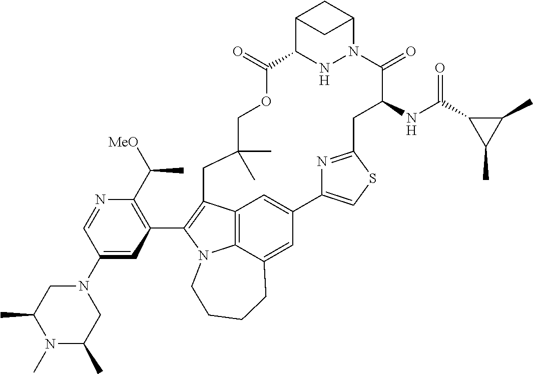

Example 2

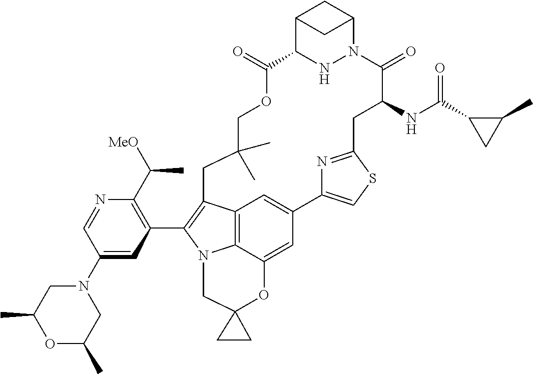

Example 3

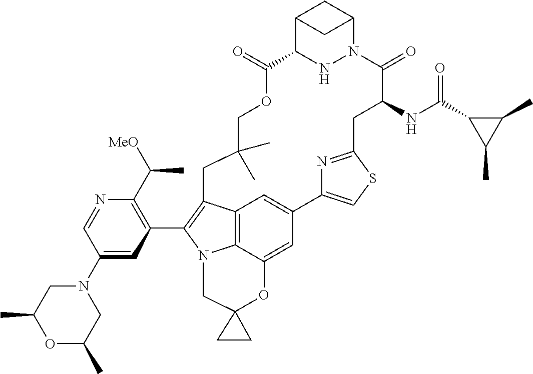

Example 4

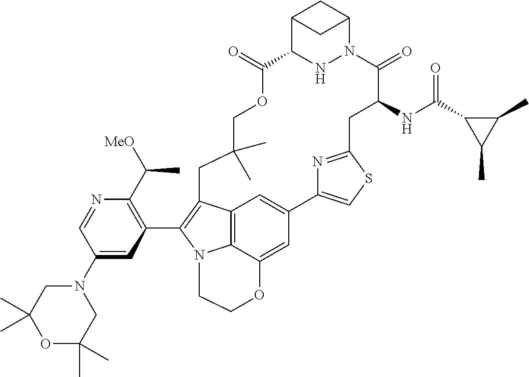

Example 5

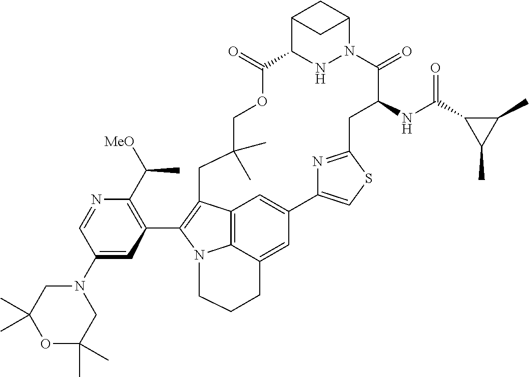

Example 6

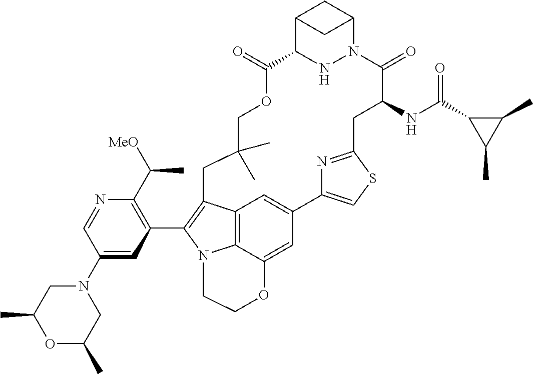

Example 7

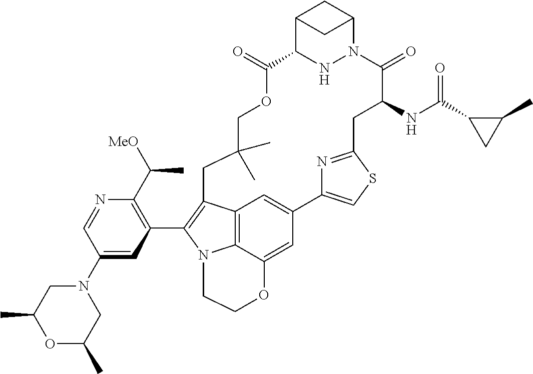

Example 8

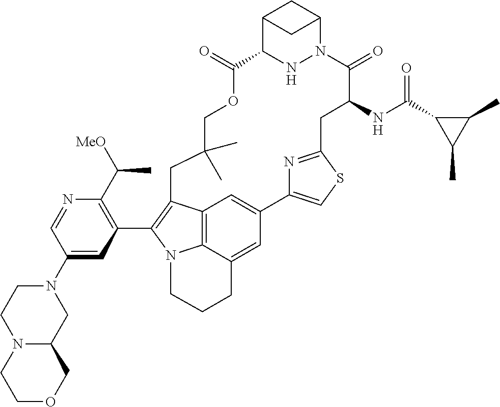

Example 9

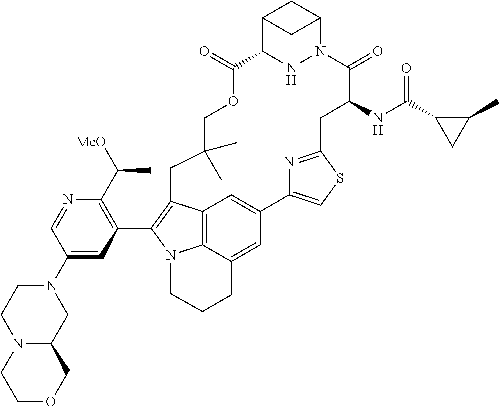

Example 10

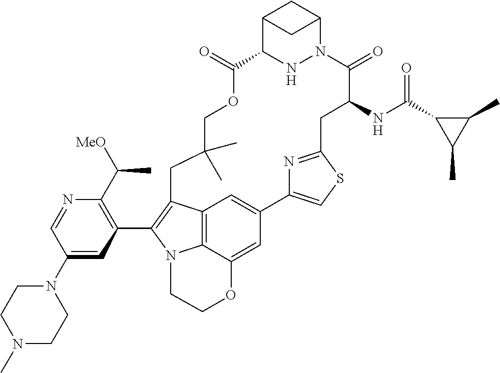

Example 11

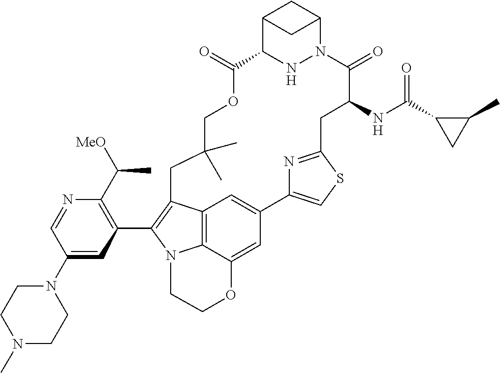

Example 12

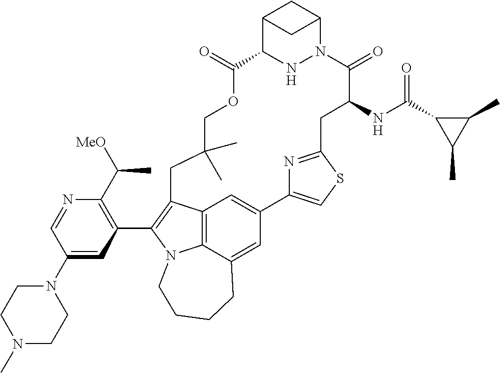

Example 14

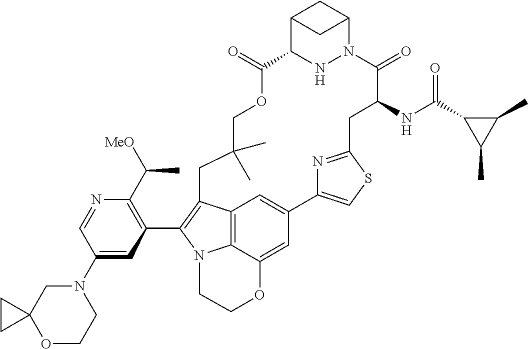

Exaple 15

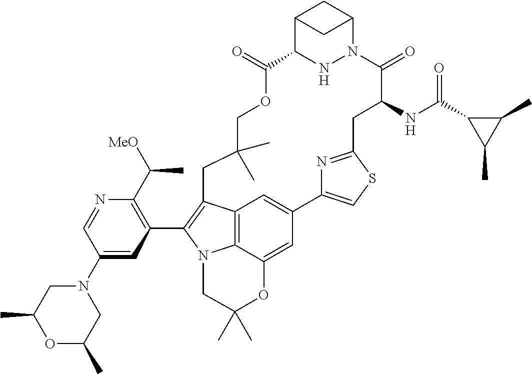

Example 16

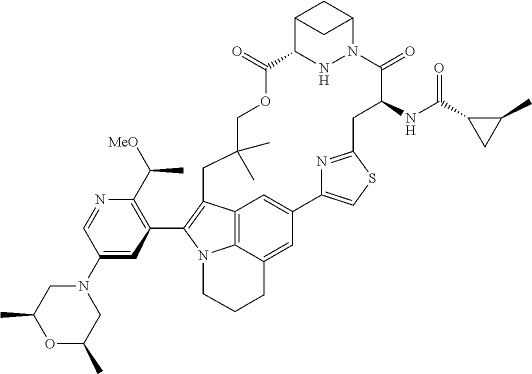

Example 17

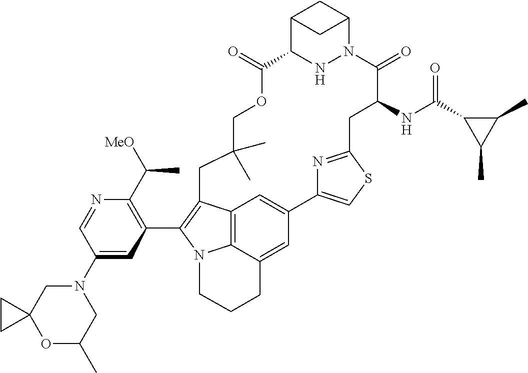

Example 18

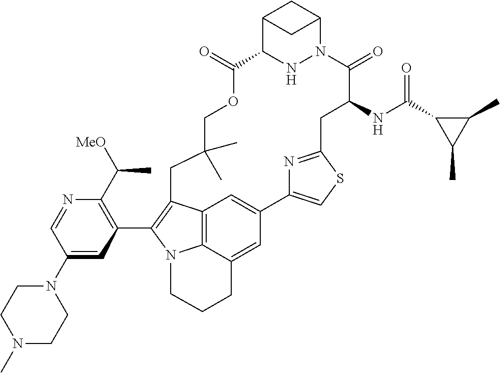

Example 19

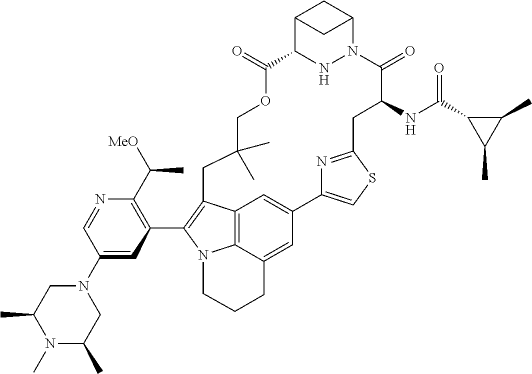

Example 20

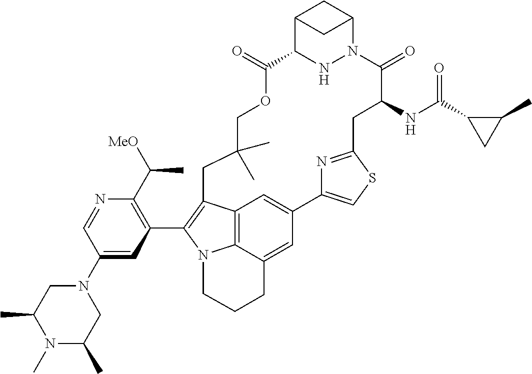

Example 21

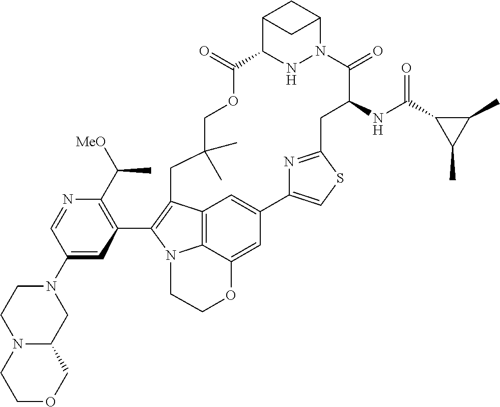

Example 22

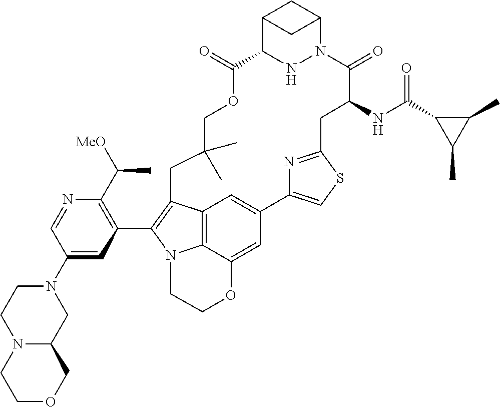

Example 23

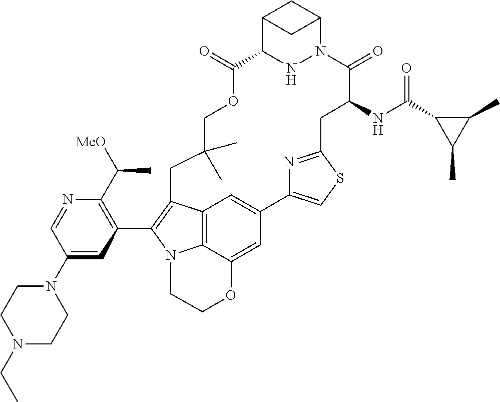

Example 24

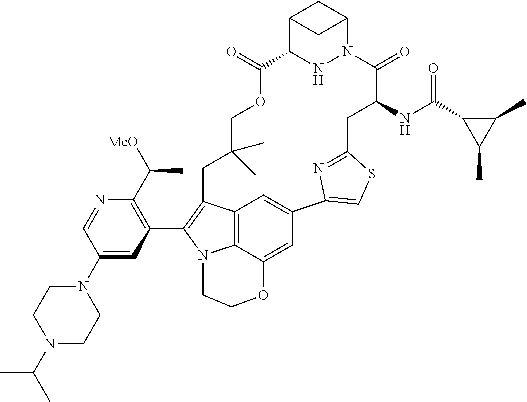

Example 25

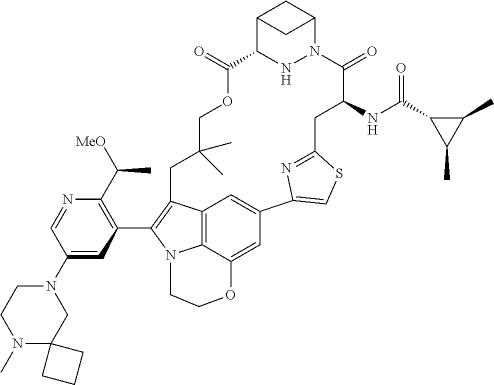

Example 26

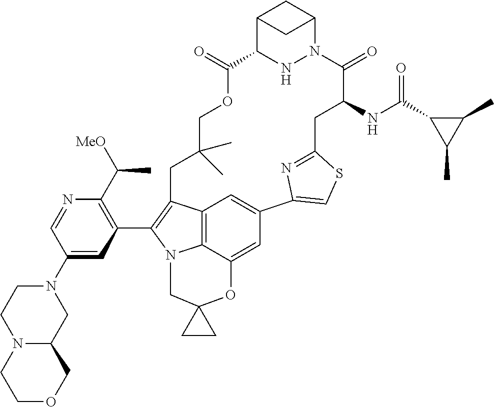

Example 27

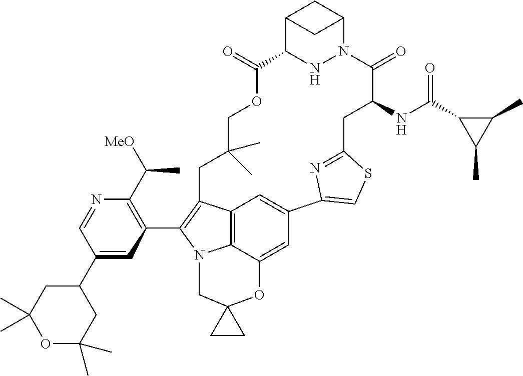

Example 28

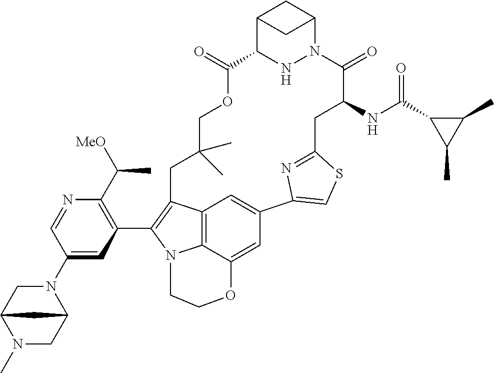

Example 29

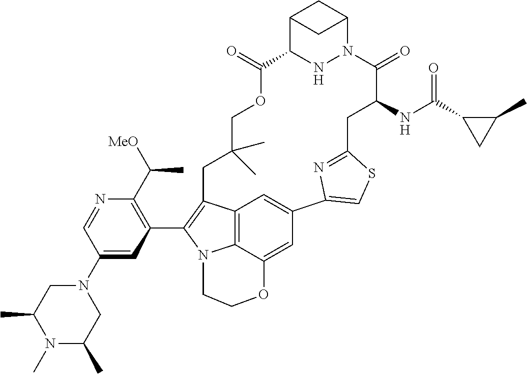

Example 30

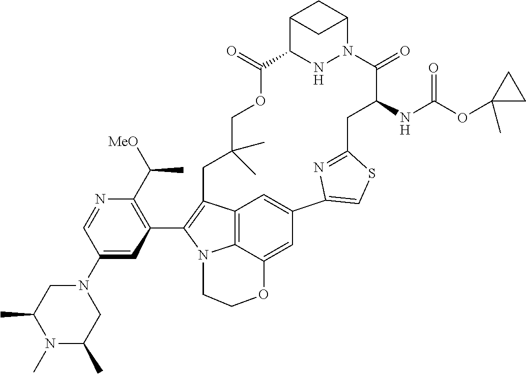

Example 31

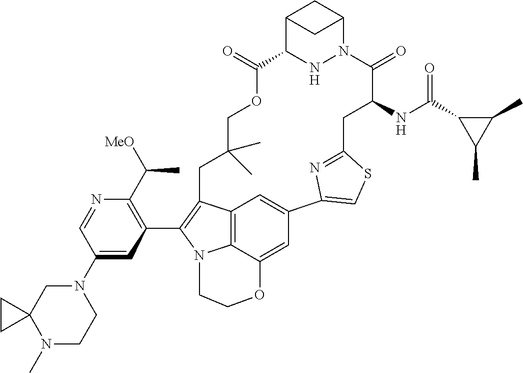

Example 32

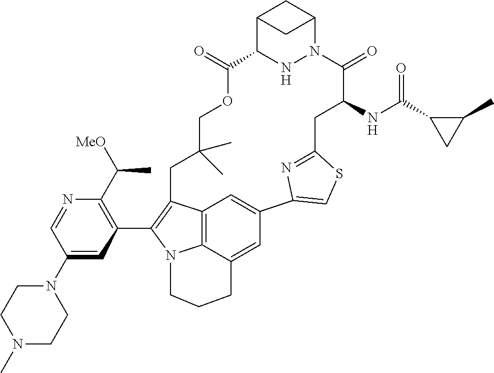

Example 33

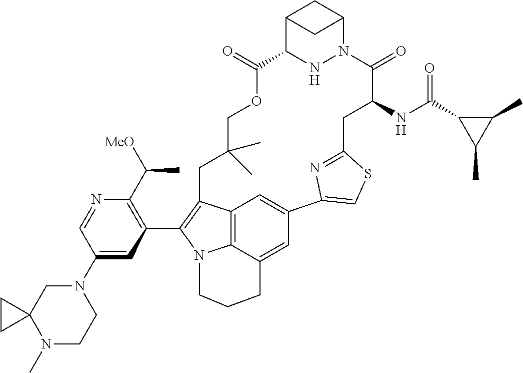

Example 34

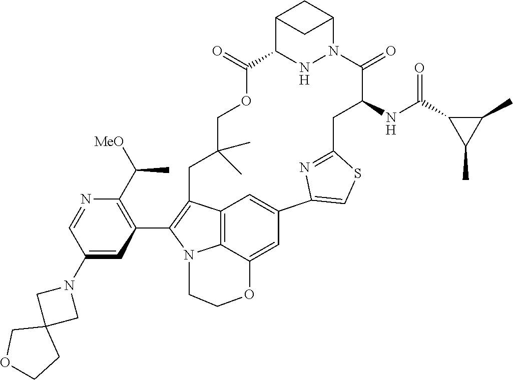

Example 35

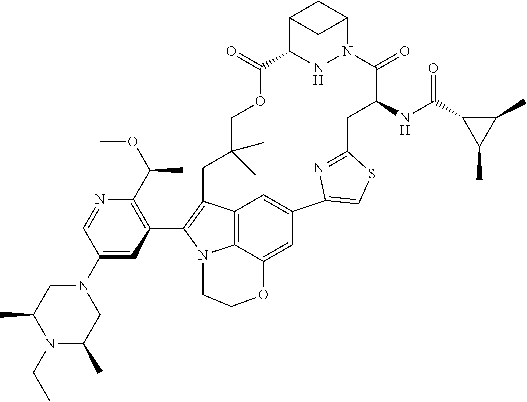

Example 36

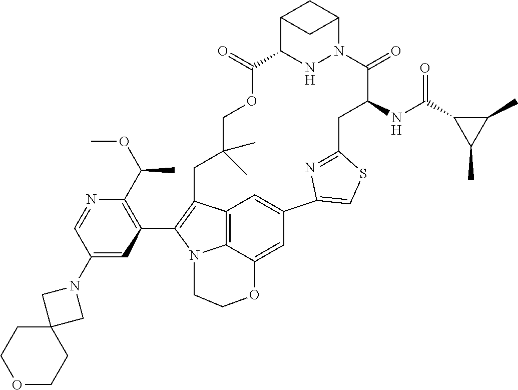

Example 37

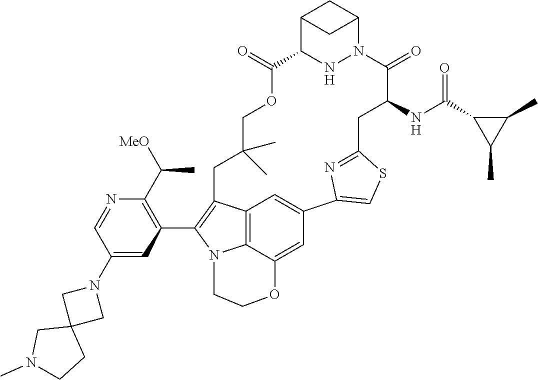

Example 38

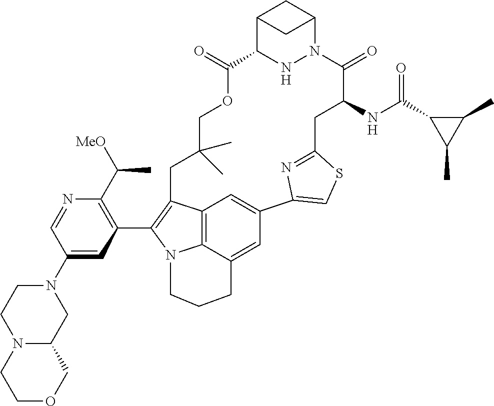

Example 39

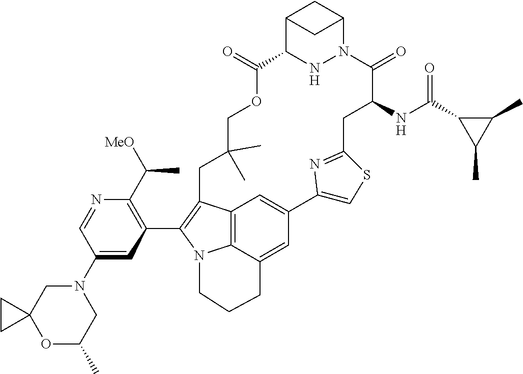

Example 40

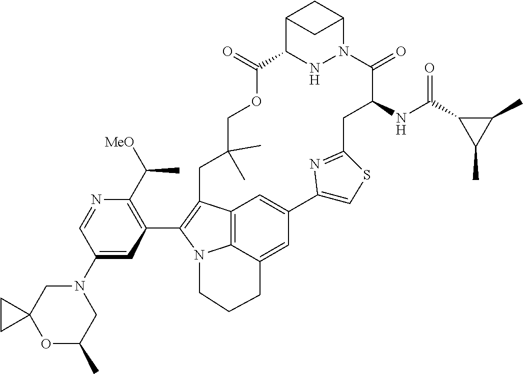

Example 41

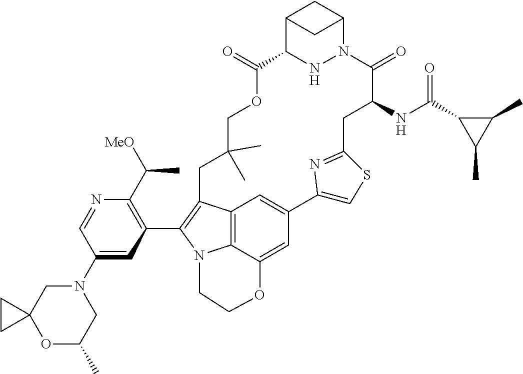

Example 42

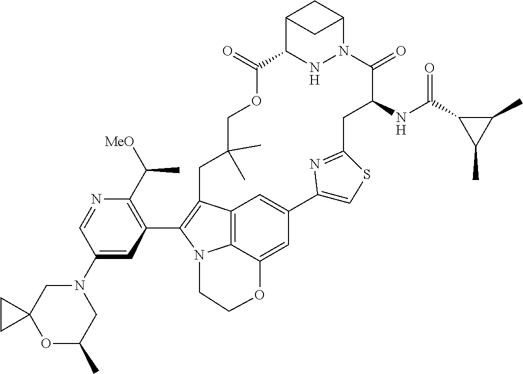

Example 43

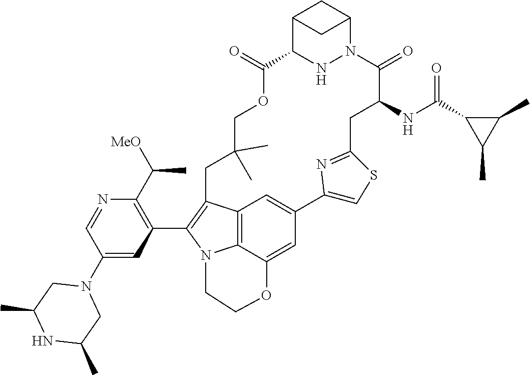

Example 44

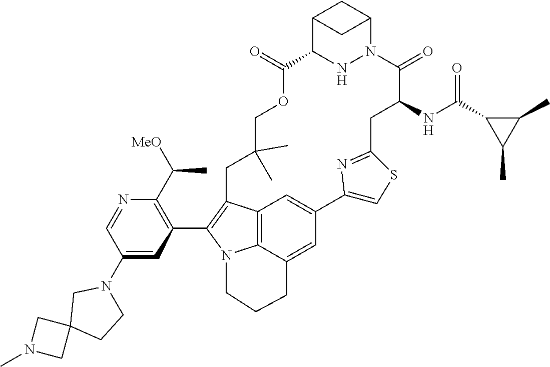

Example 45

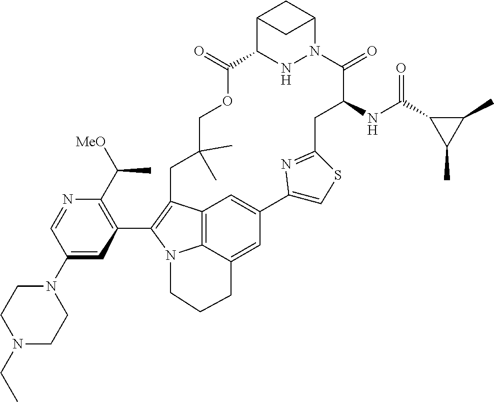

Example 46

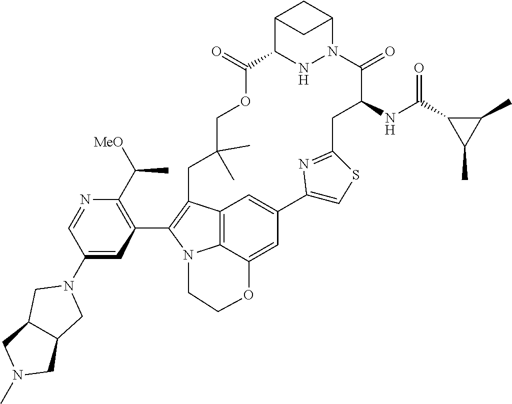

Example 47

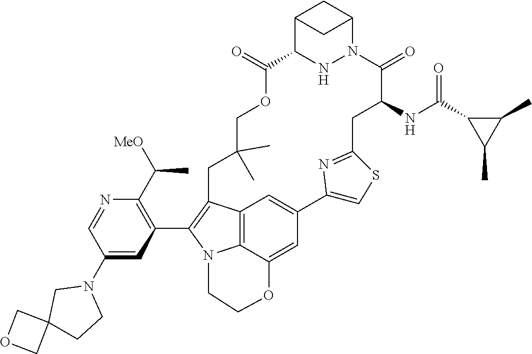

Example 48

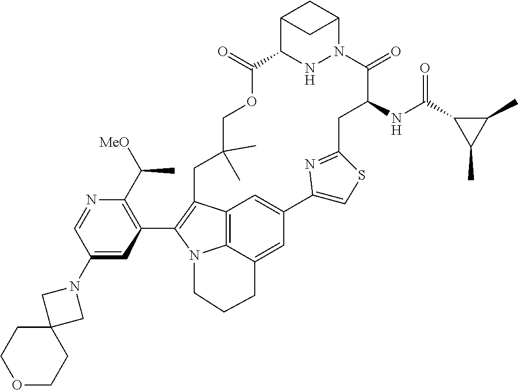

Example 49

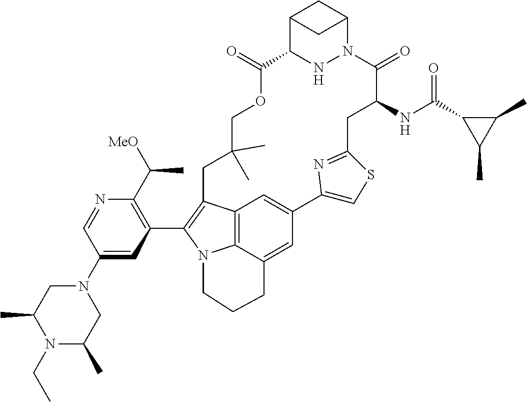

Example 50

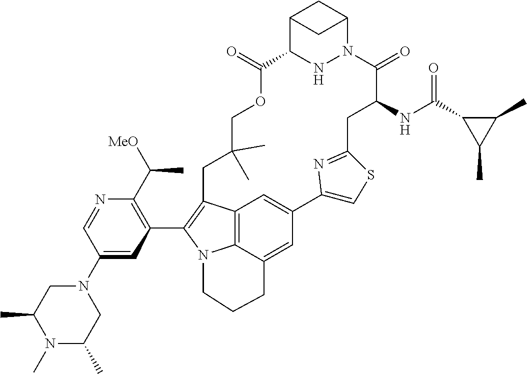

Example 51

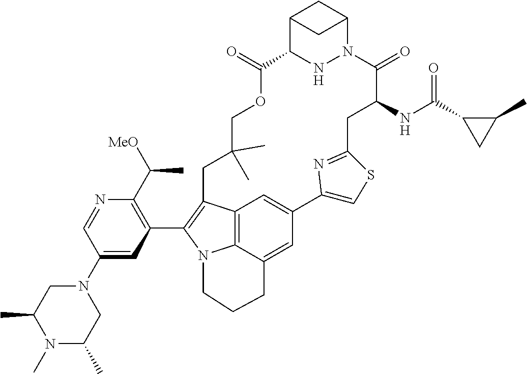

Example 52

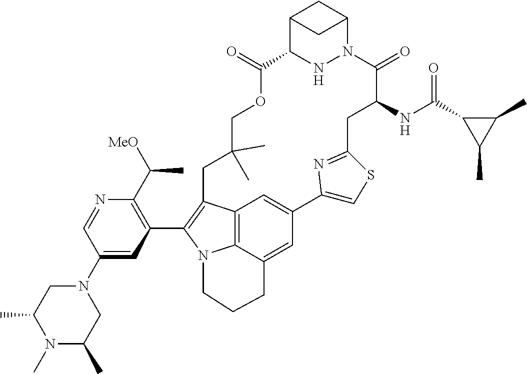

Example 53

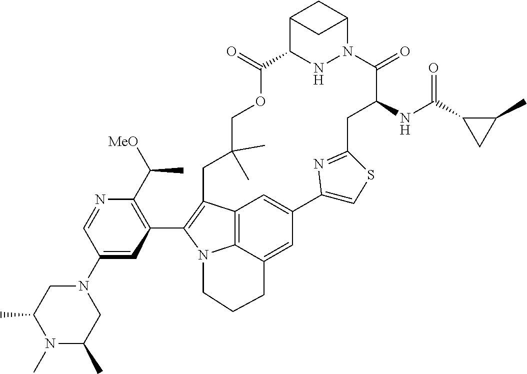

Example 54

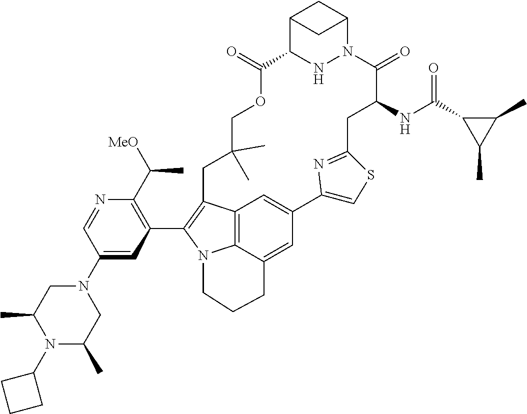

Example 55

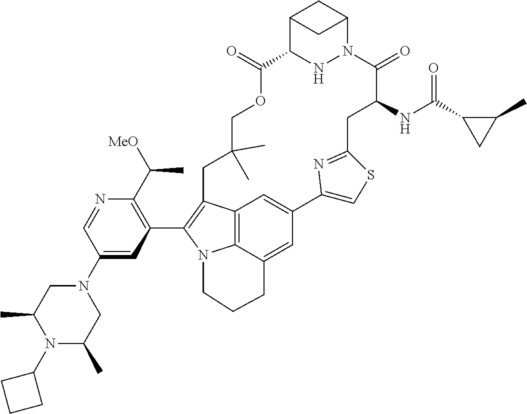

Example 56

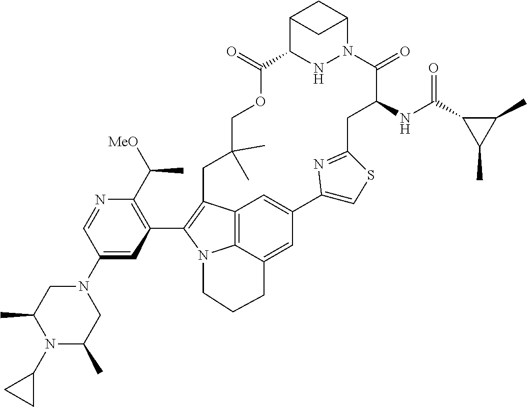

Example 57

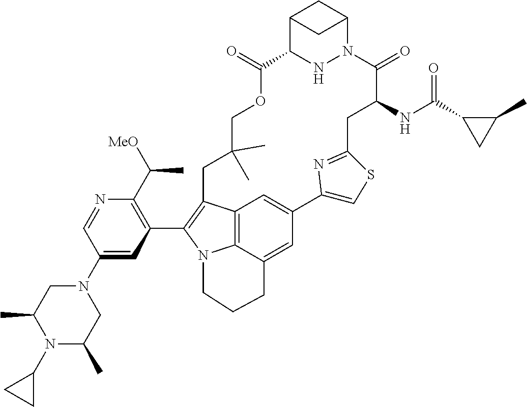

Example 58

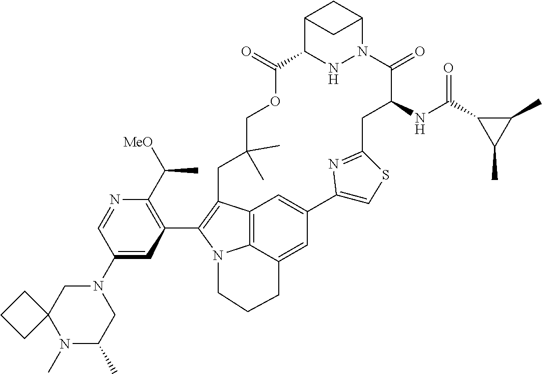

Example 59

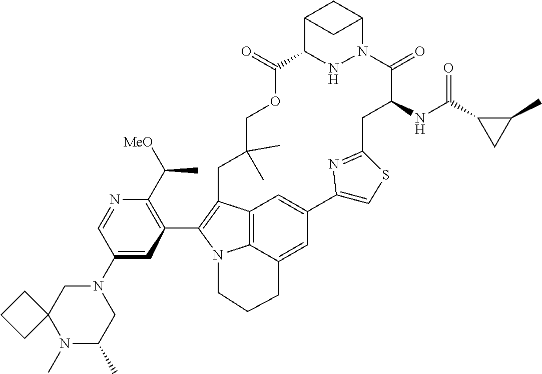

Example 60

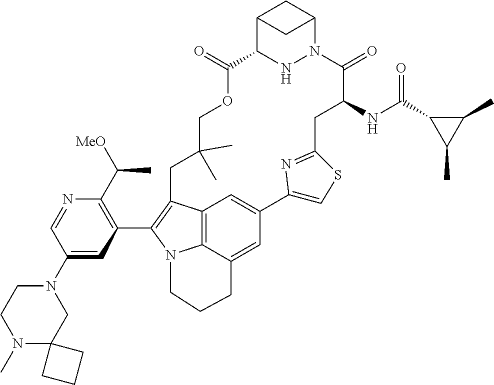

Example 61

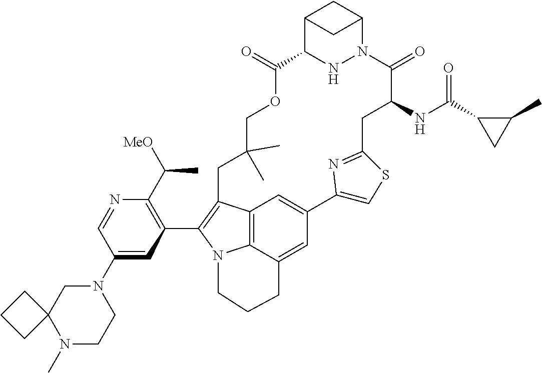

Example 62

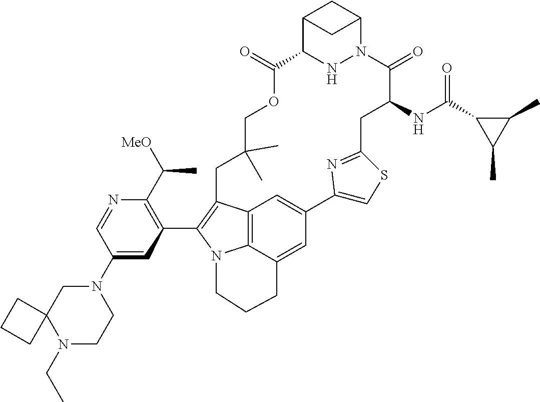

Example 63

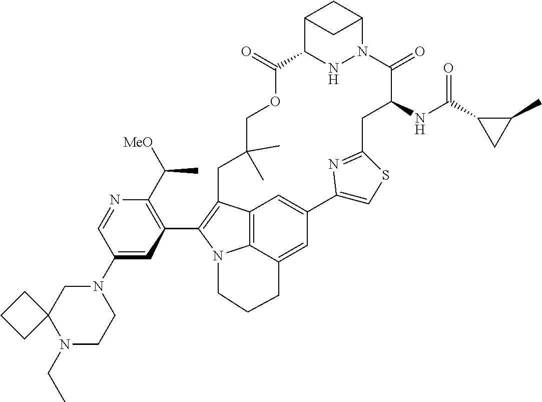

Example 64

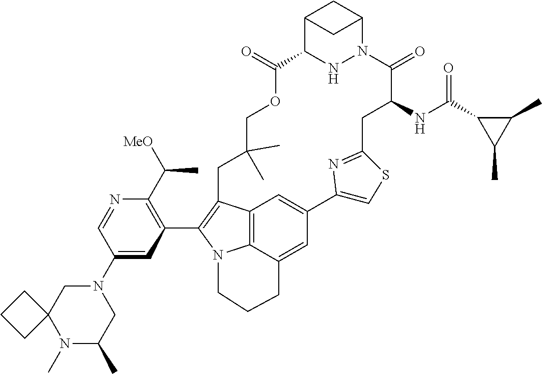

Example 65

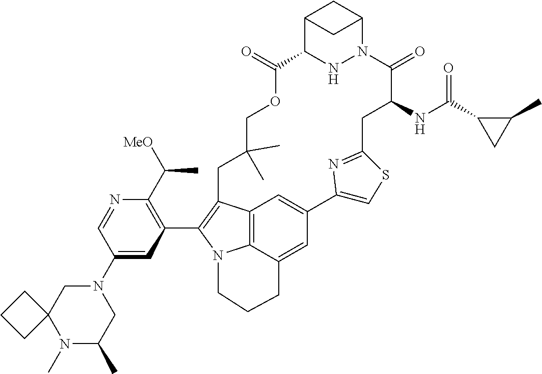

Example 66

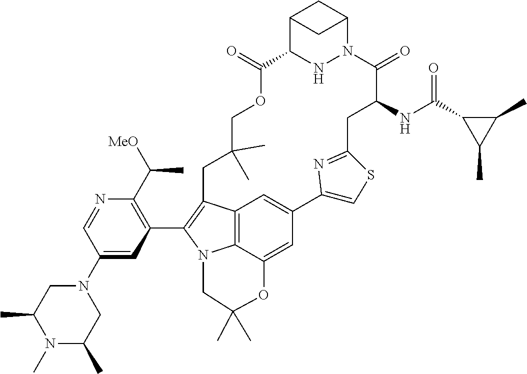

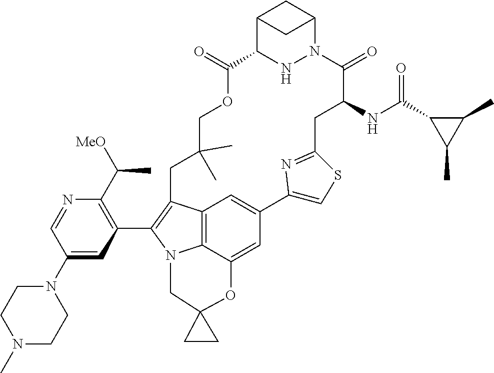

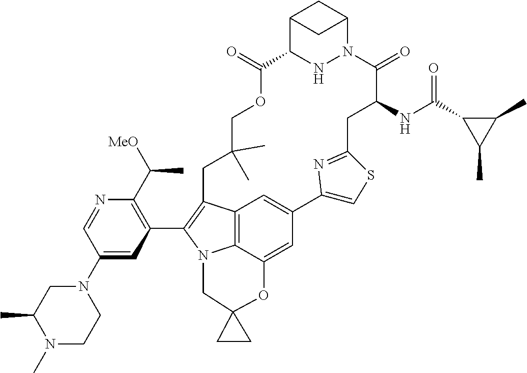

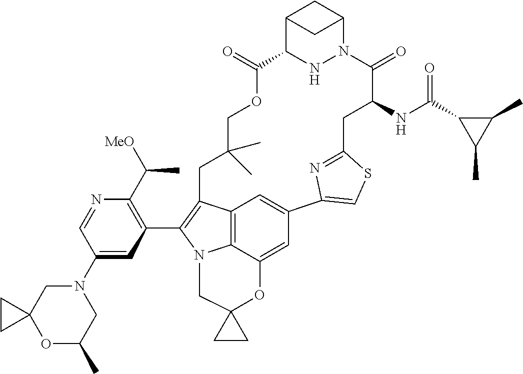

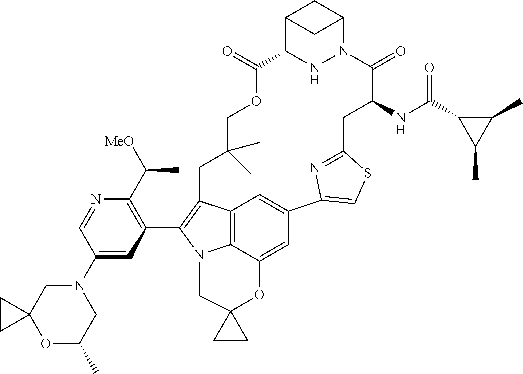

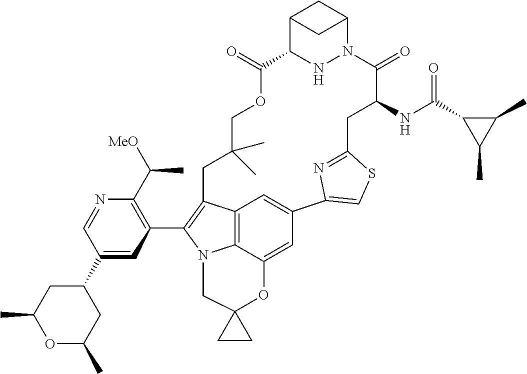

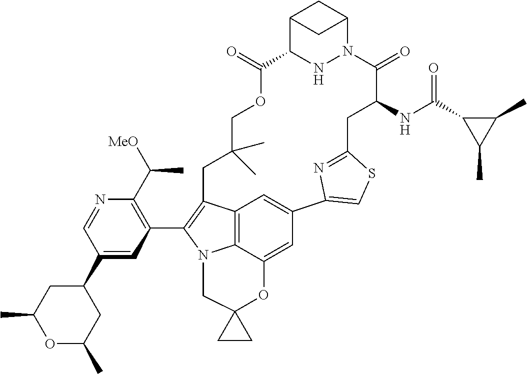

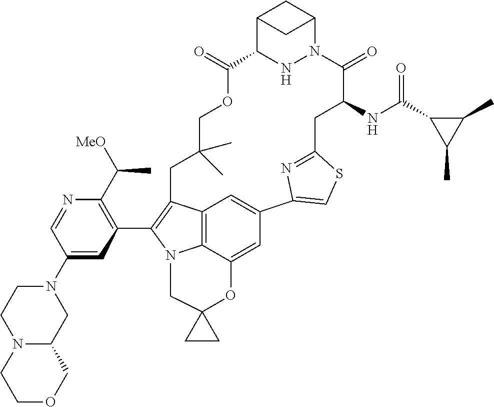

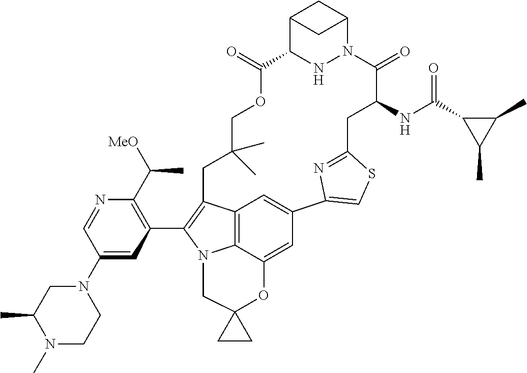

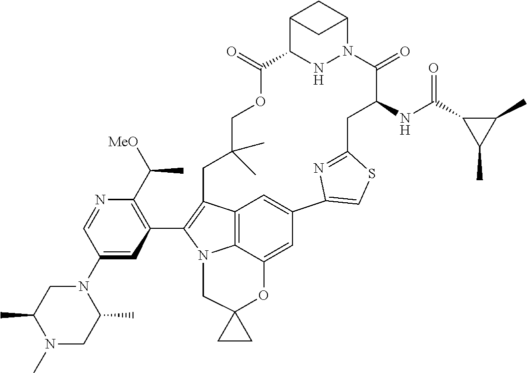

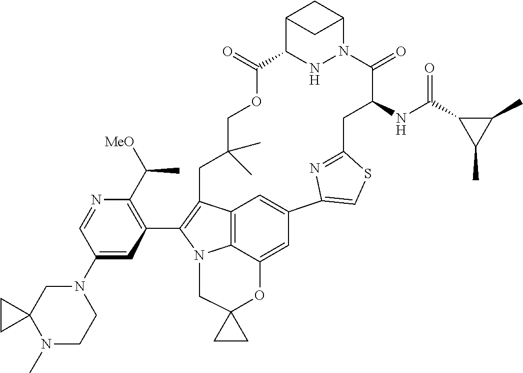

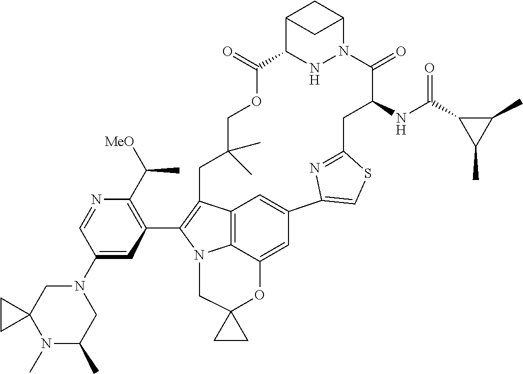

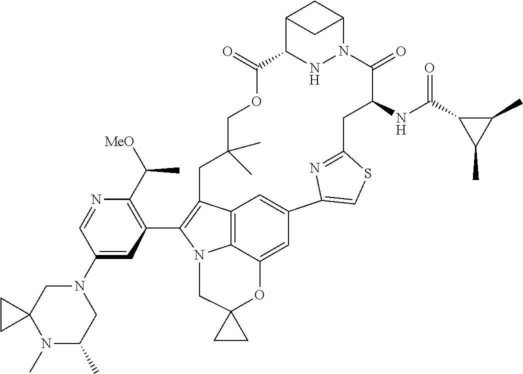

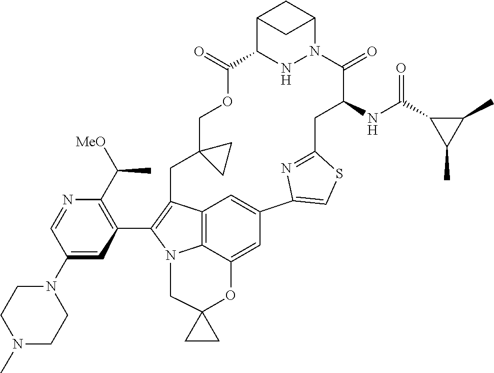

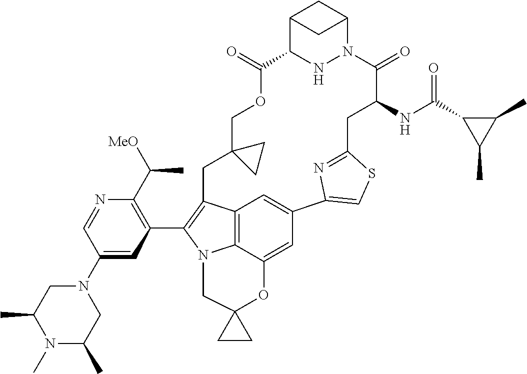

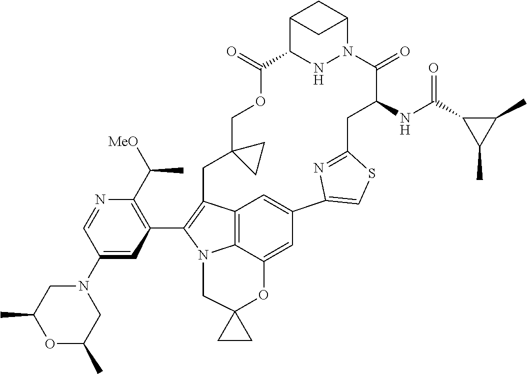

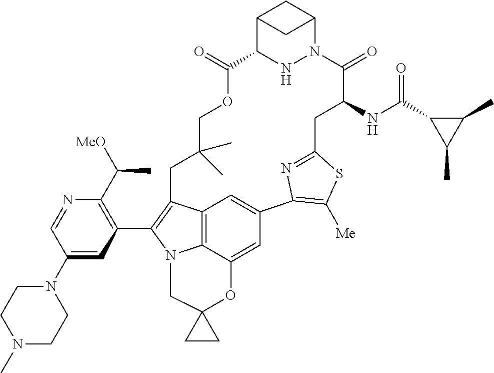

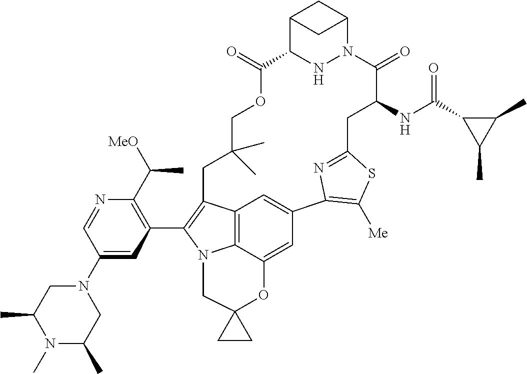

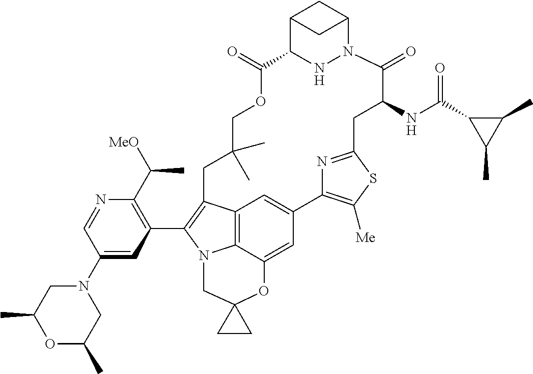

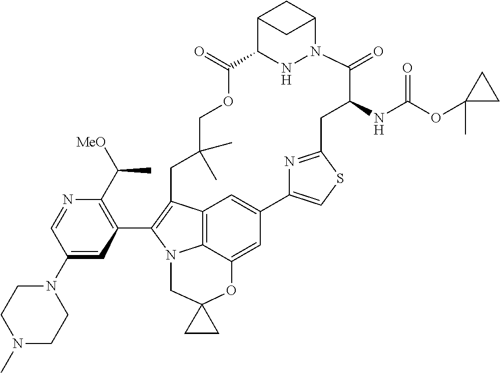

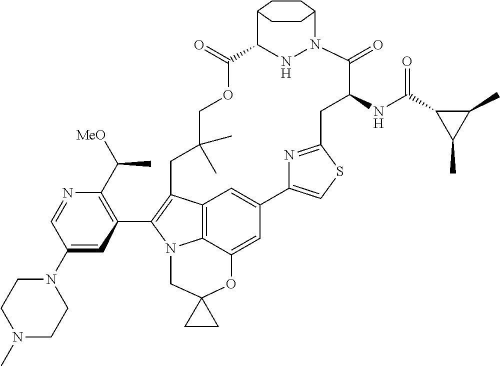

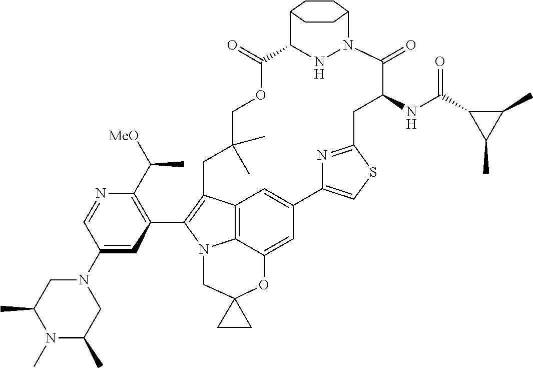

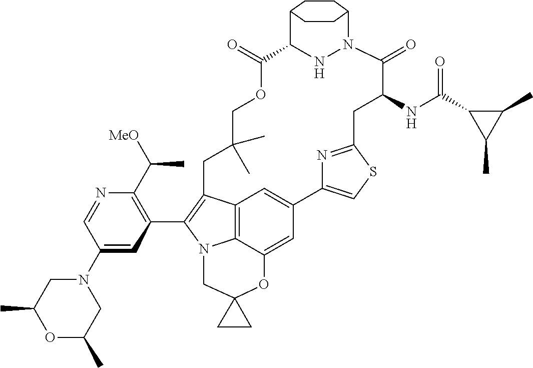

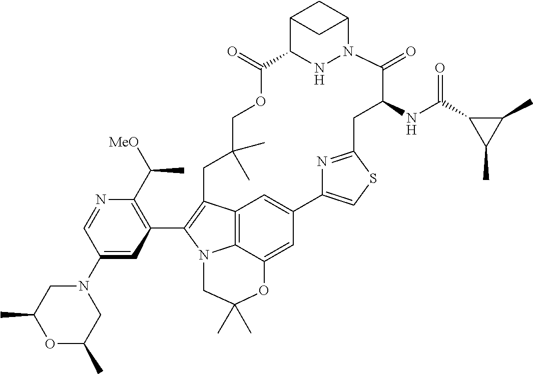

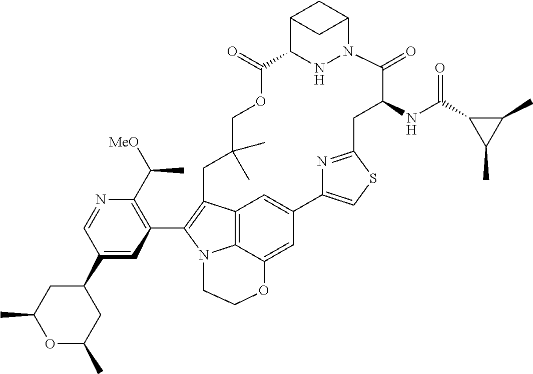

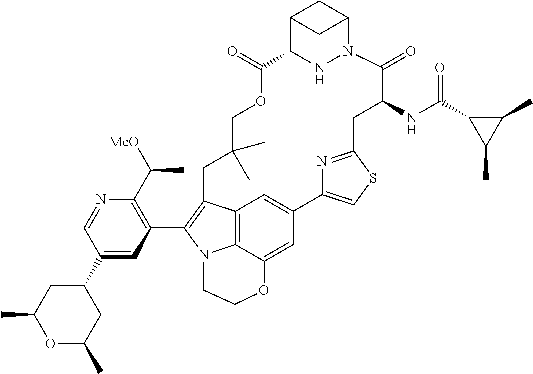

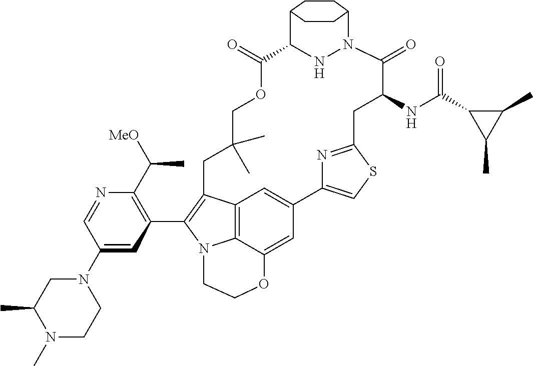

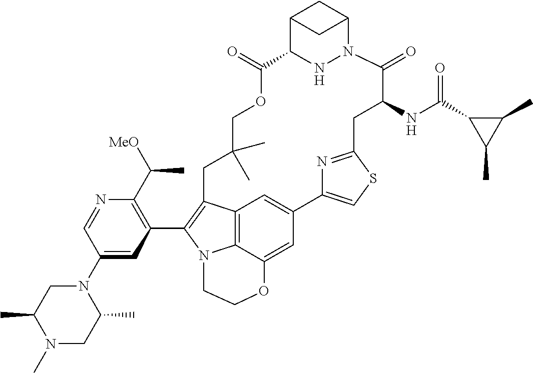

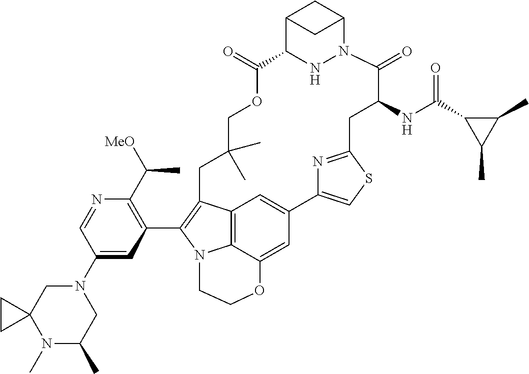

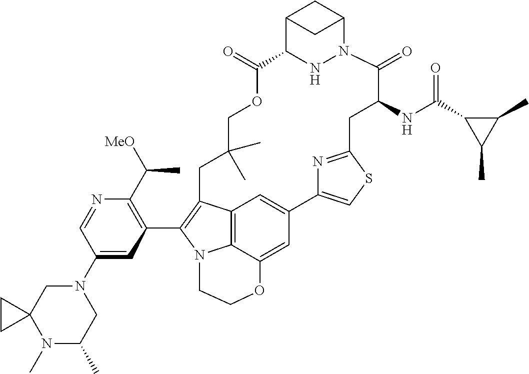

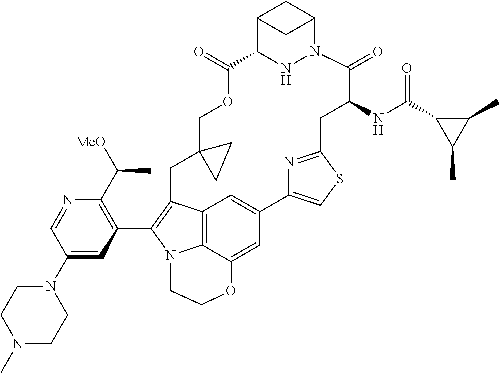

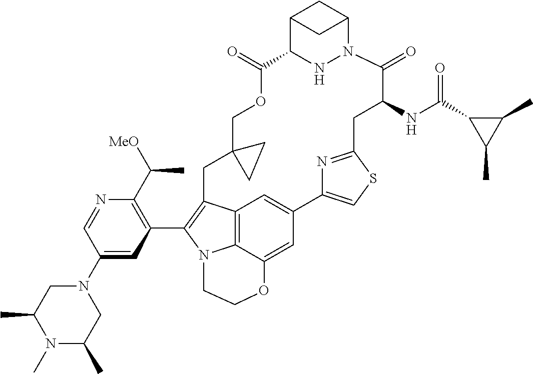

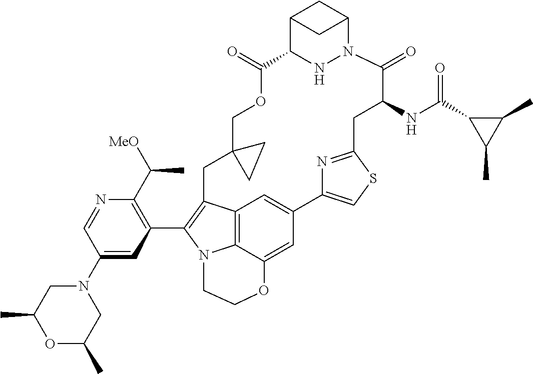

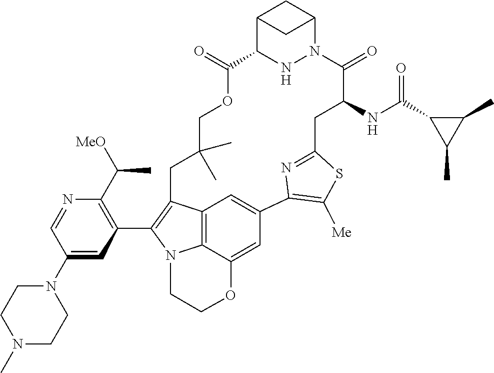

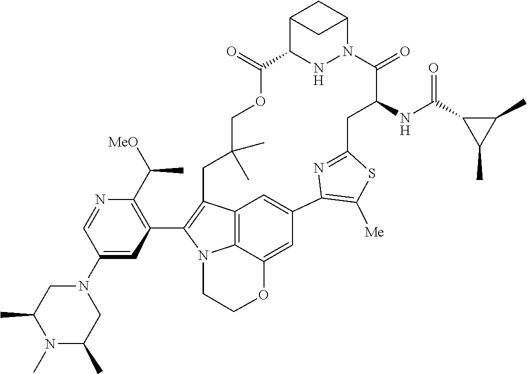

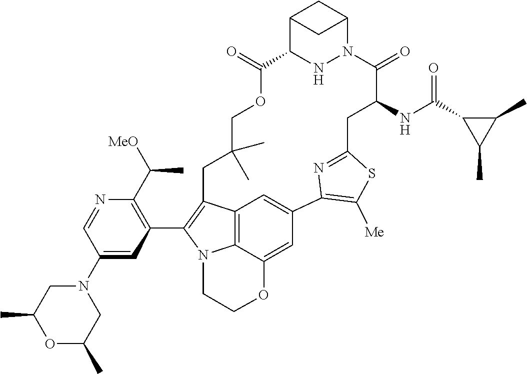

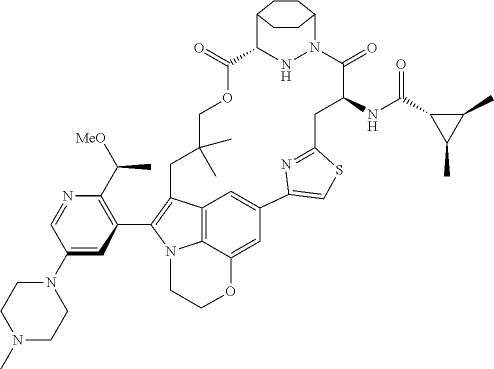

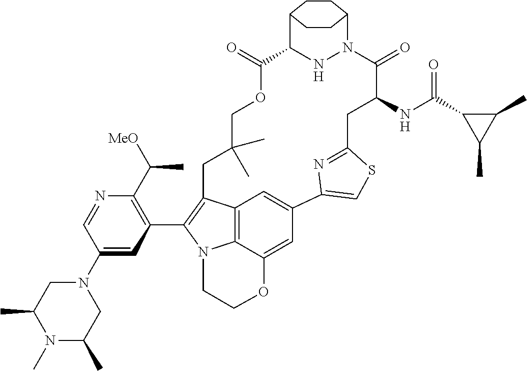

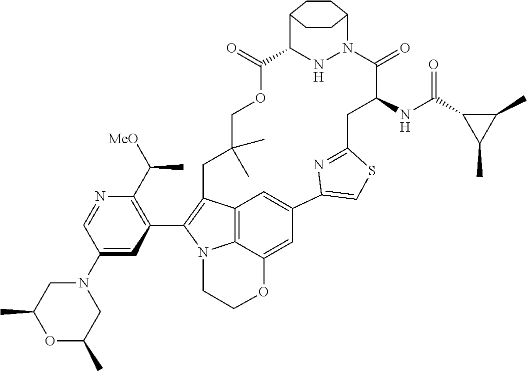

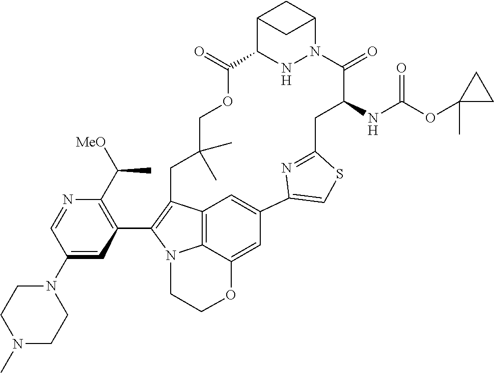

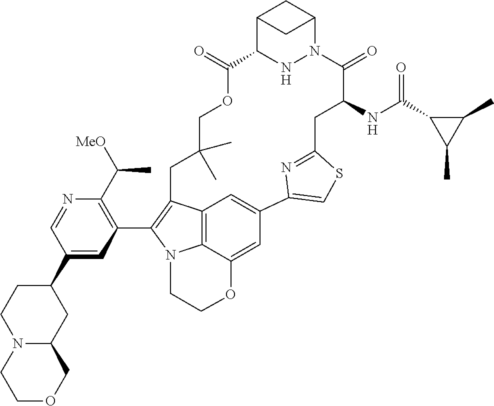

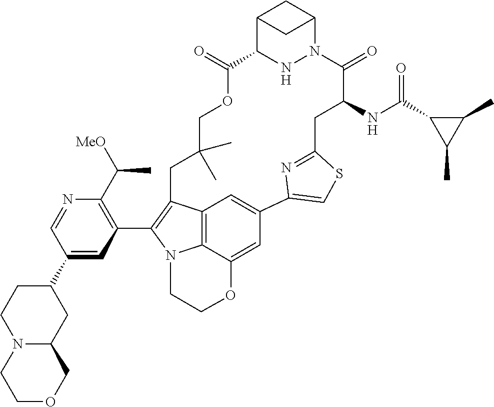

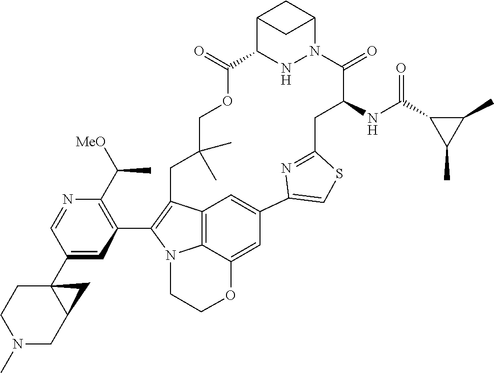

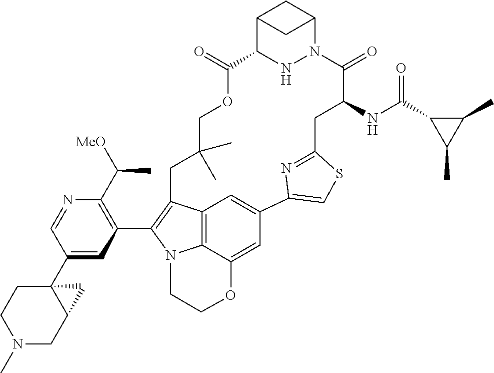

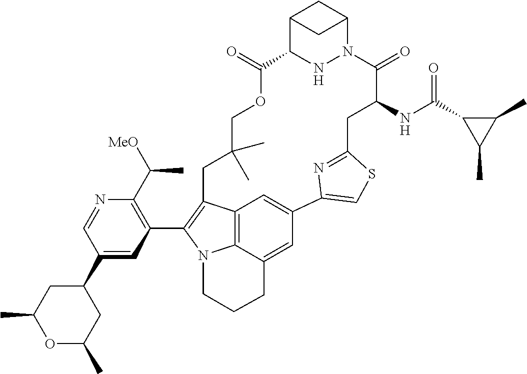

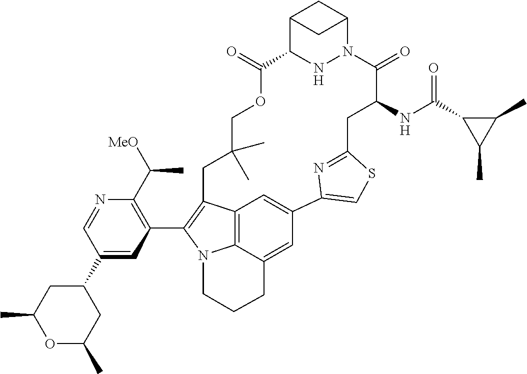

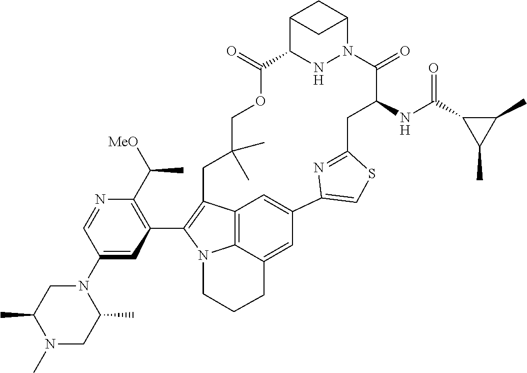

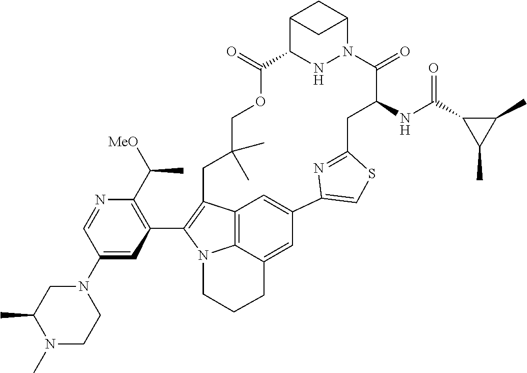

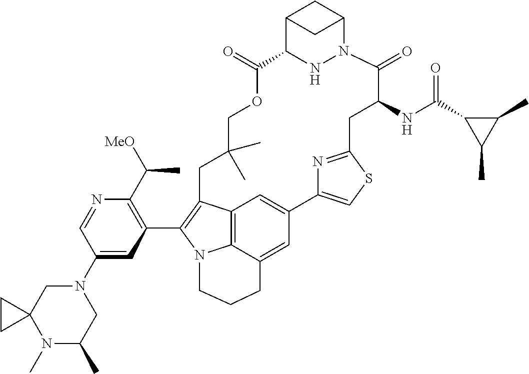

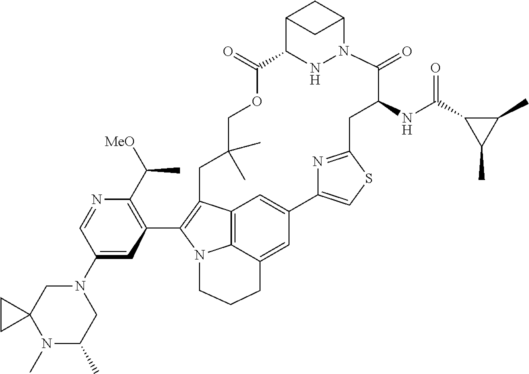

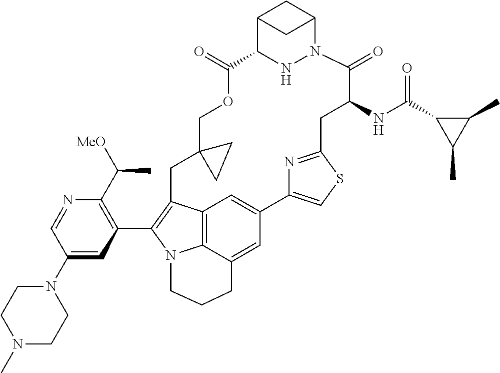

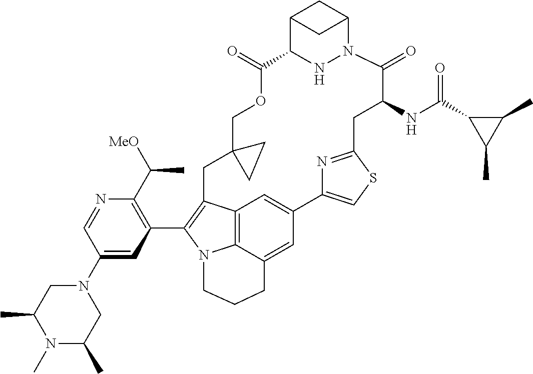

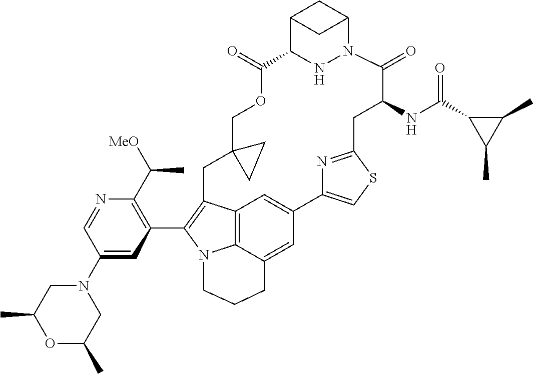

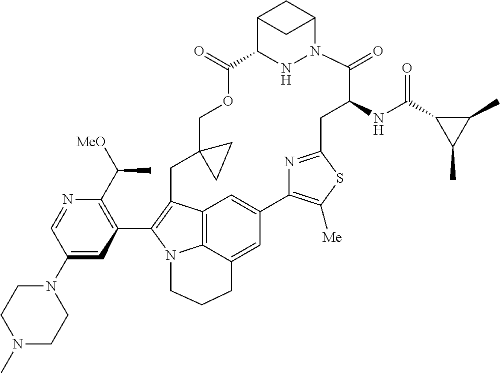

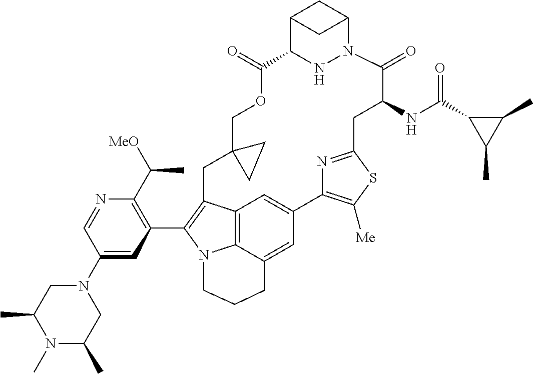

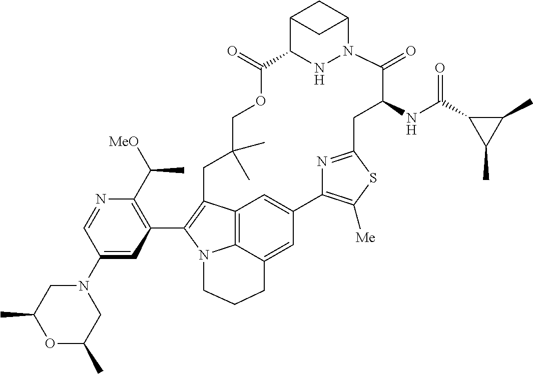

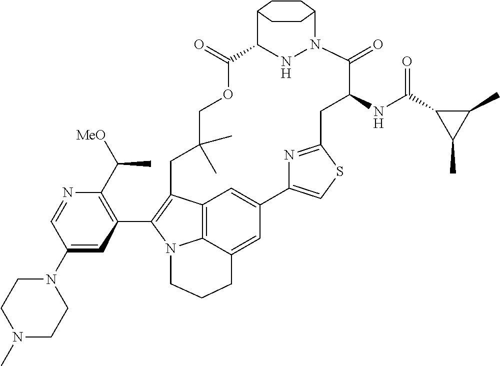

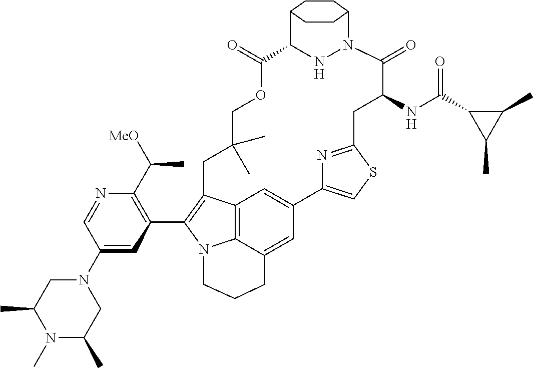

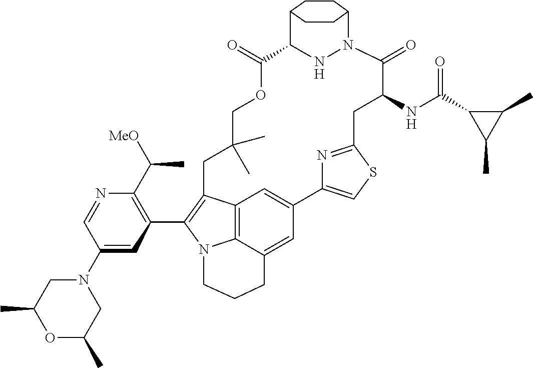

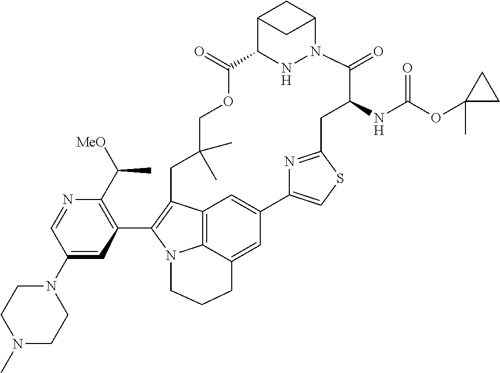

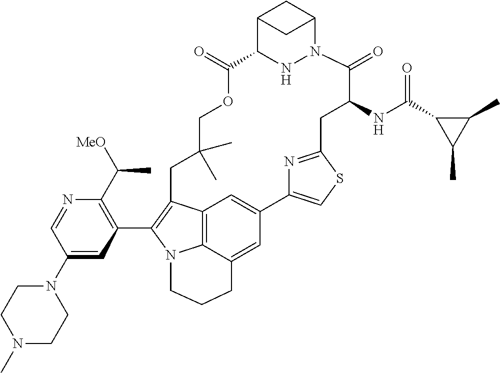

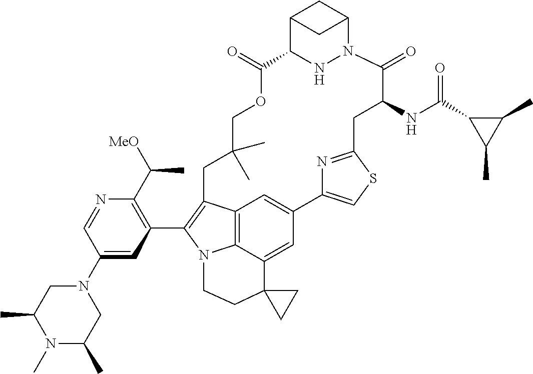

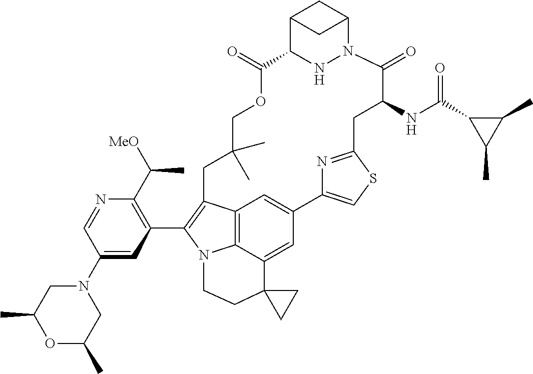

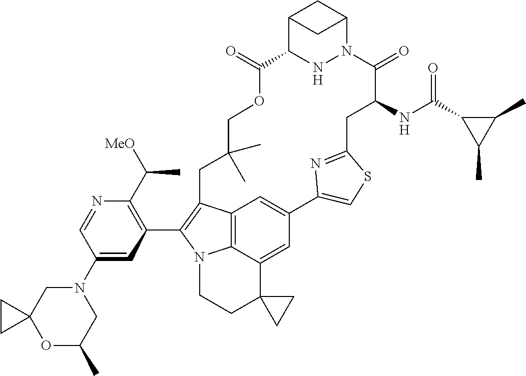

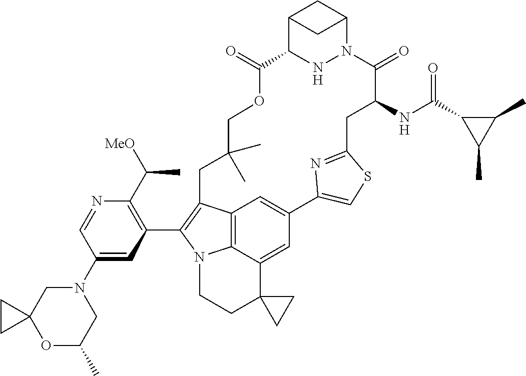

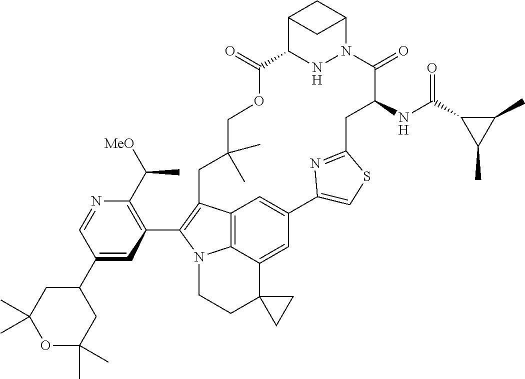

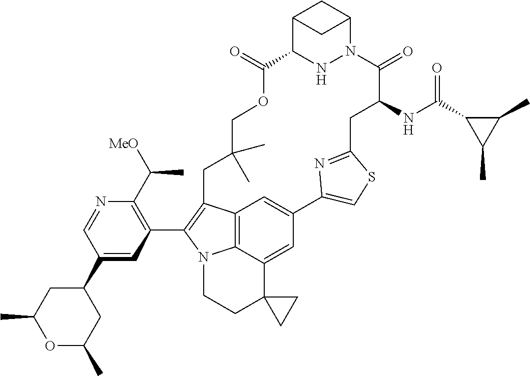

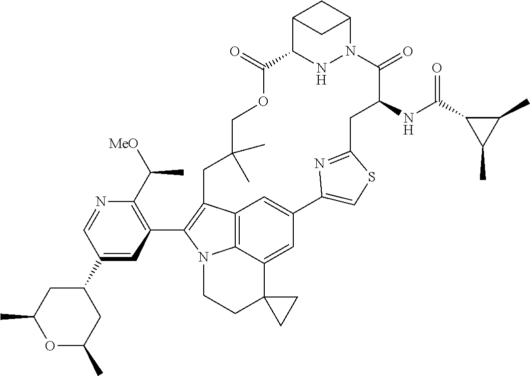

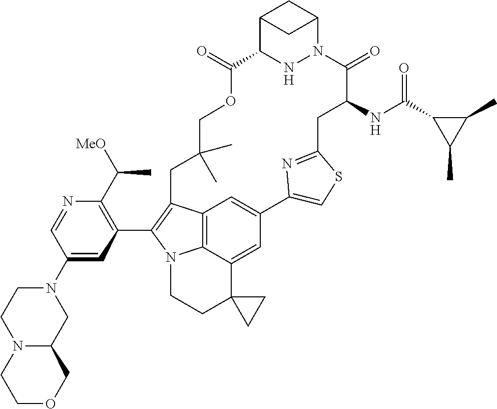

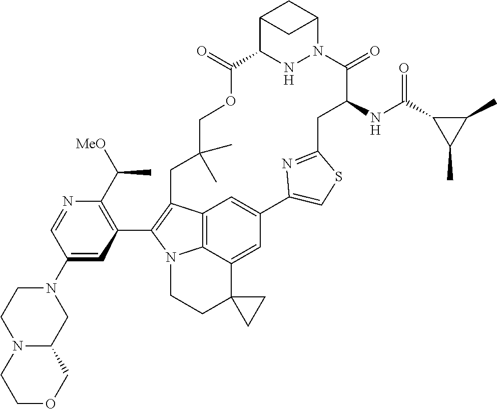

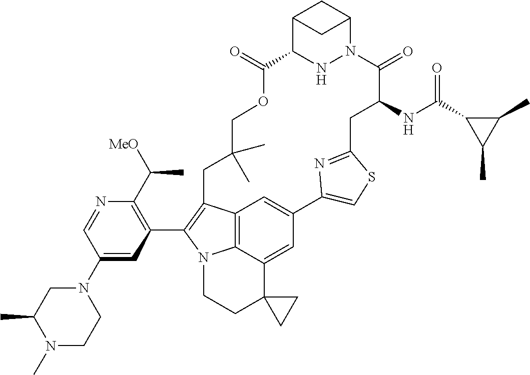

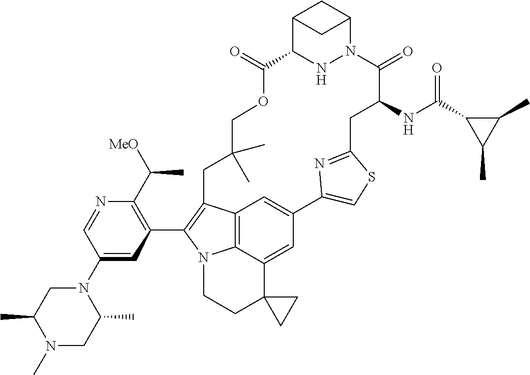

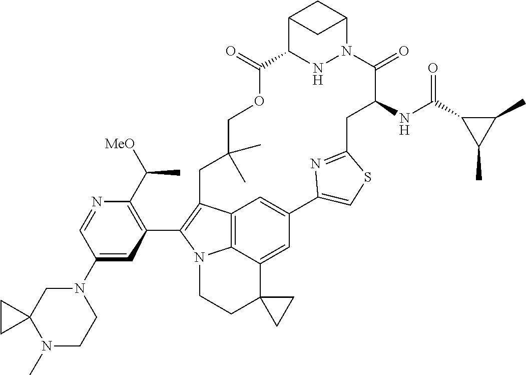

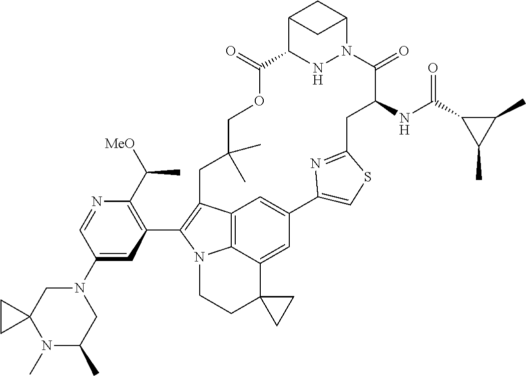

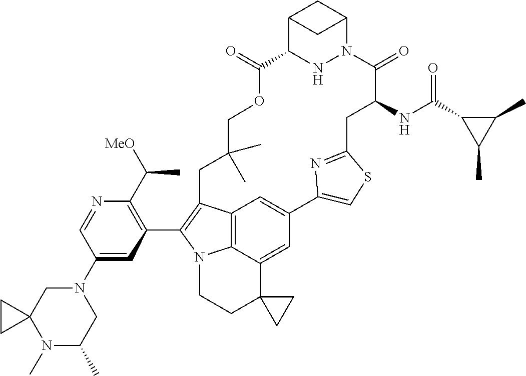

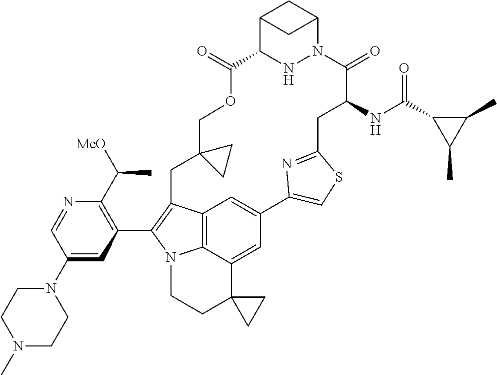

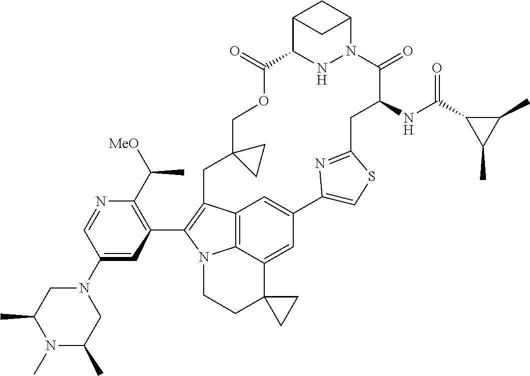

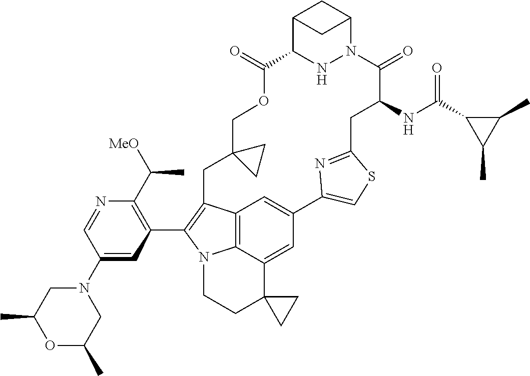

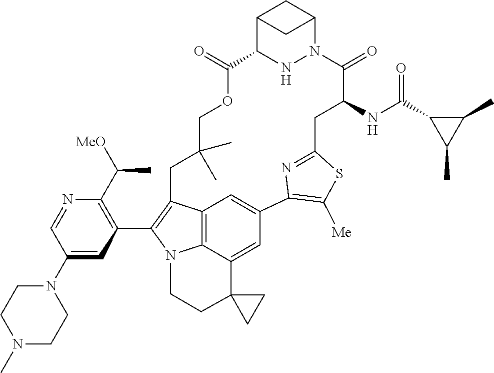

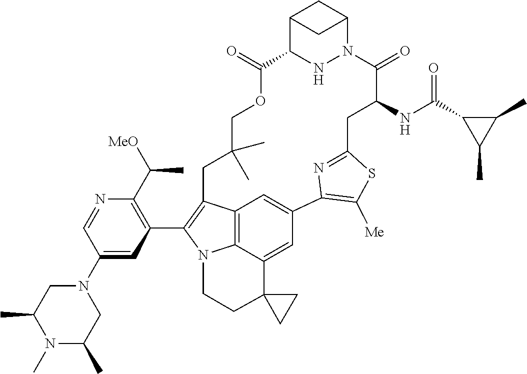

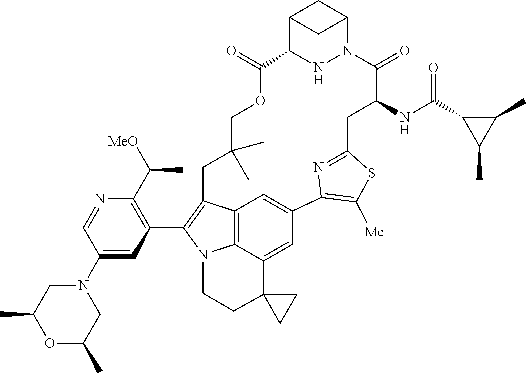

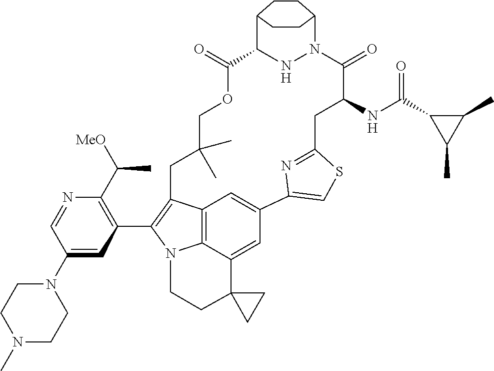

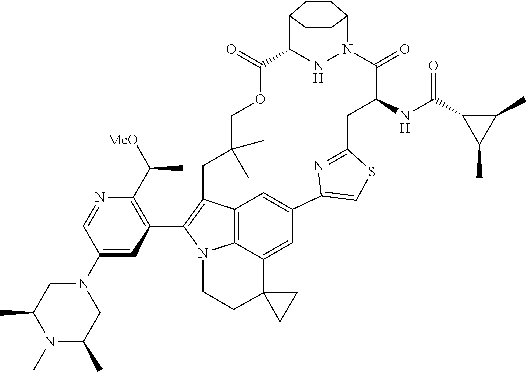

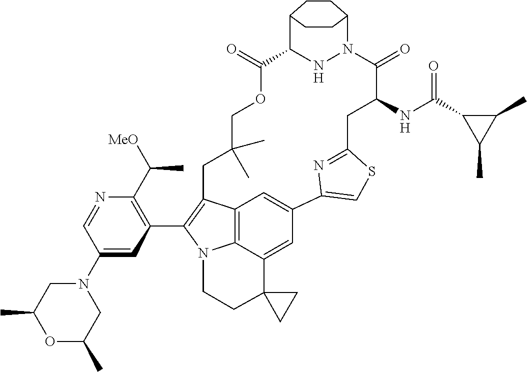

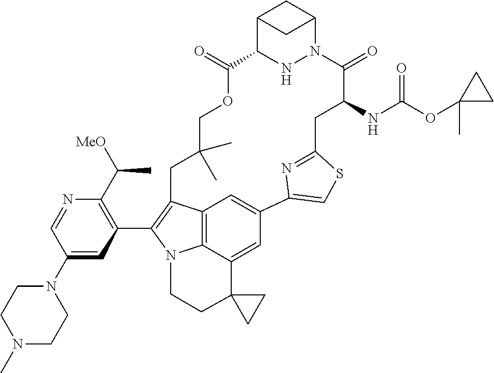

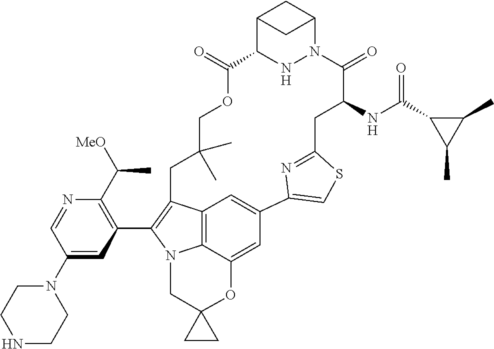

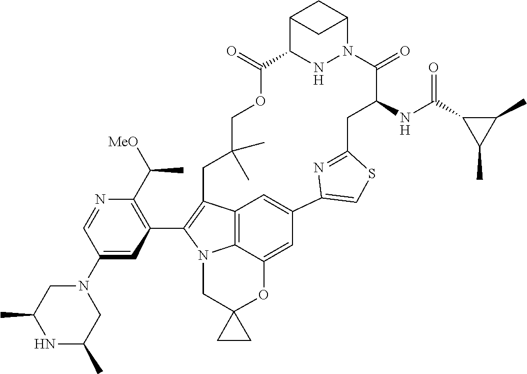

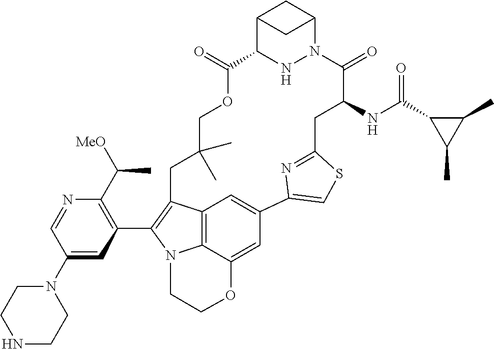

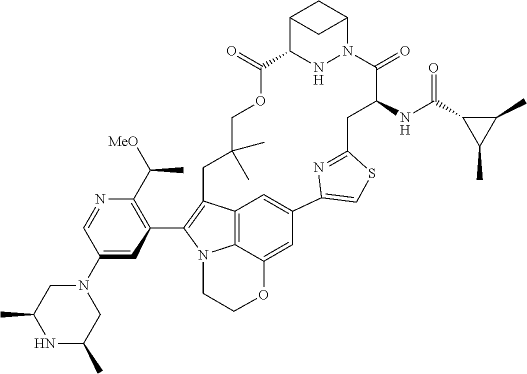

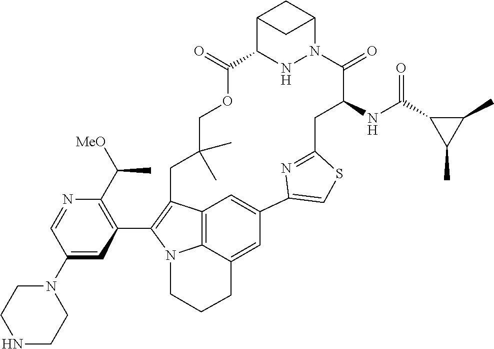

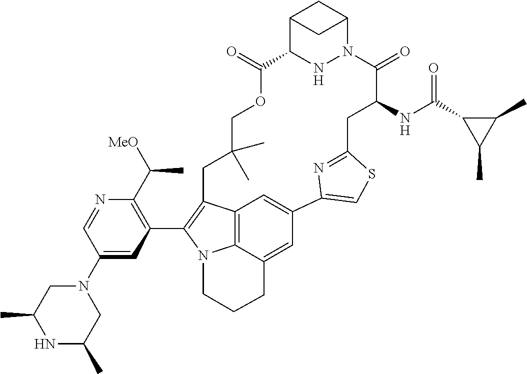

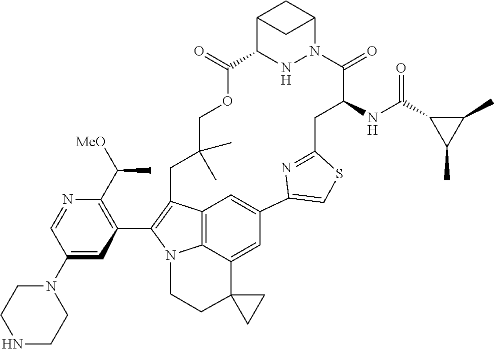

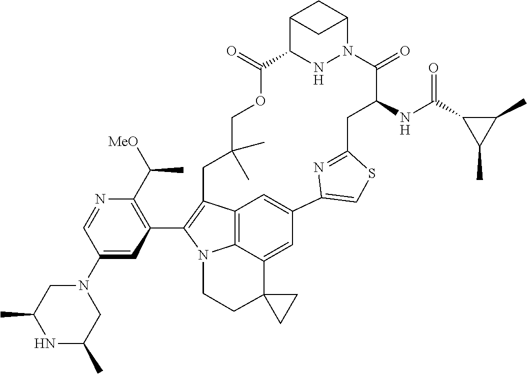

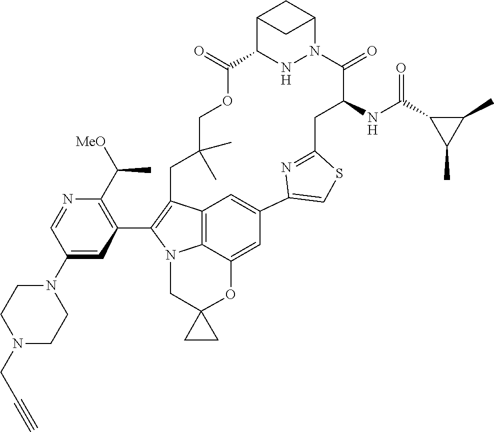

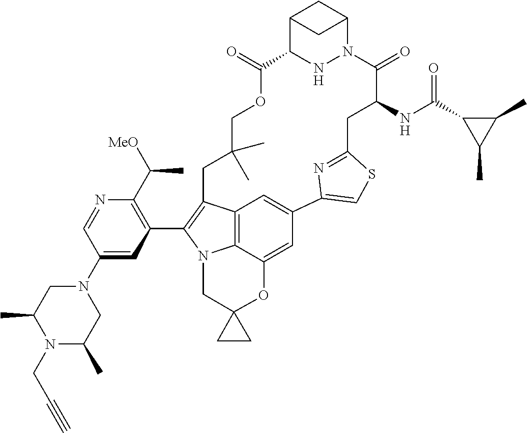

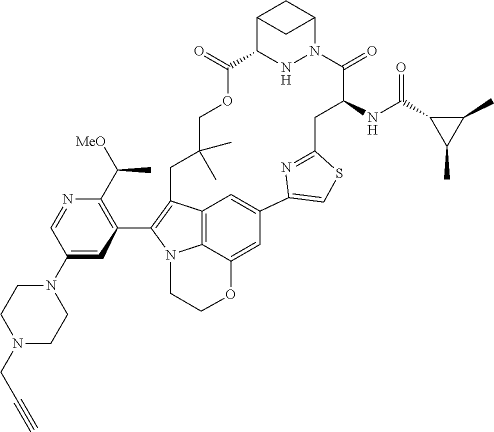

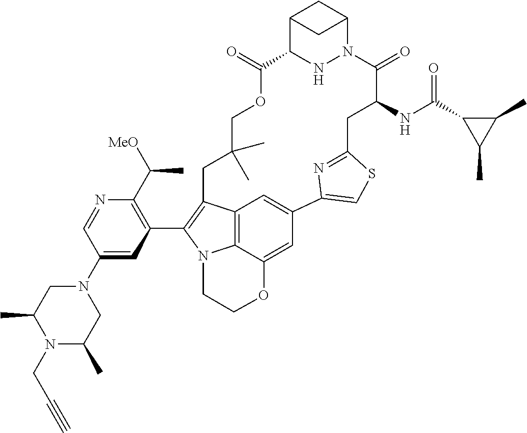

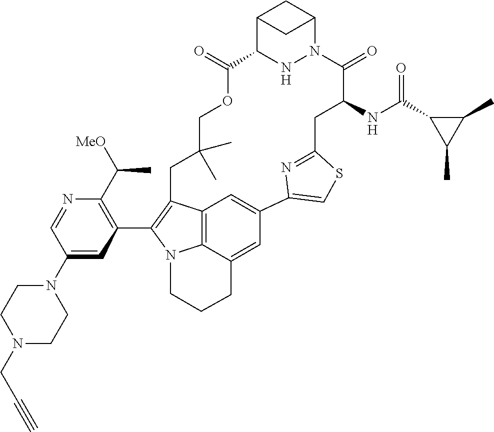

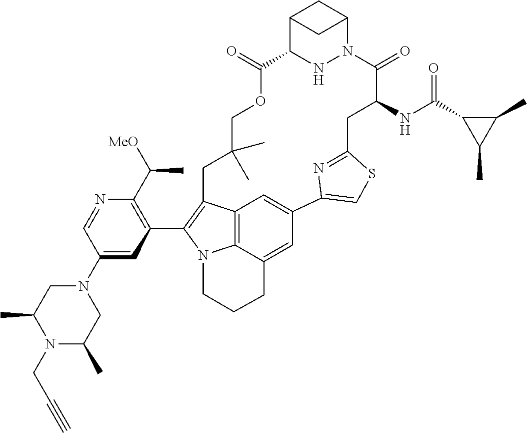

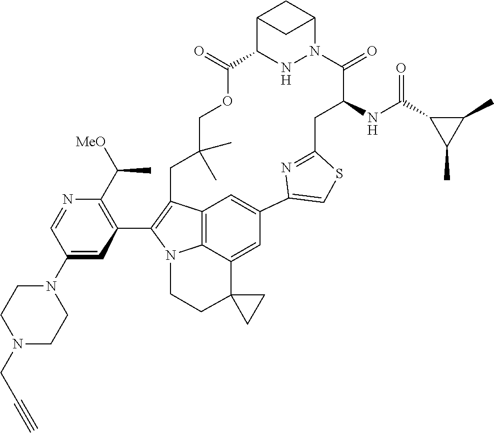

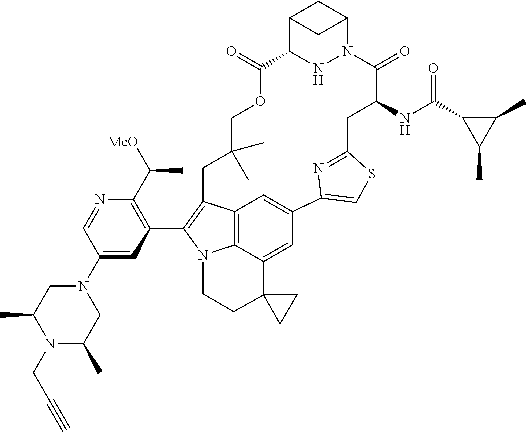

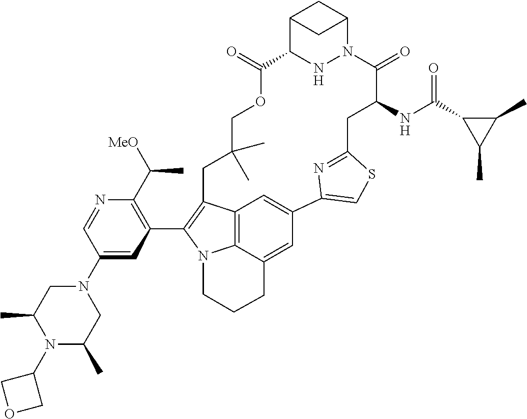

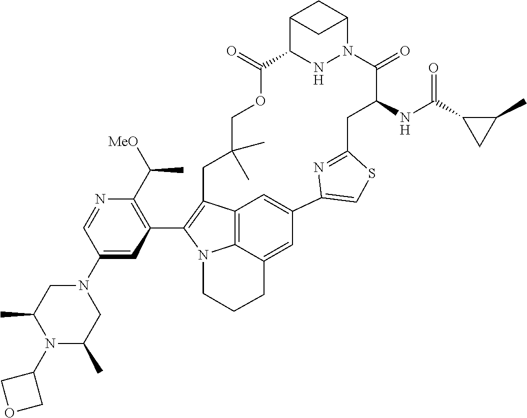

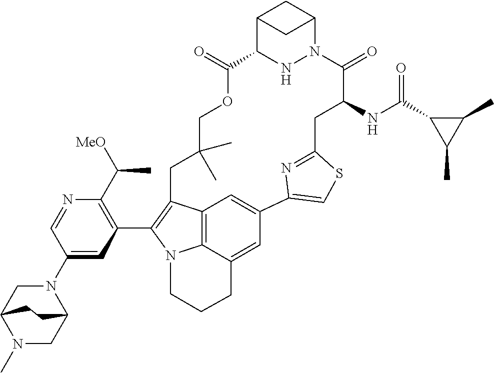

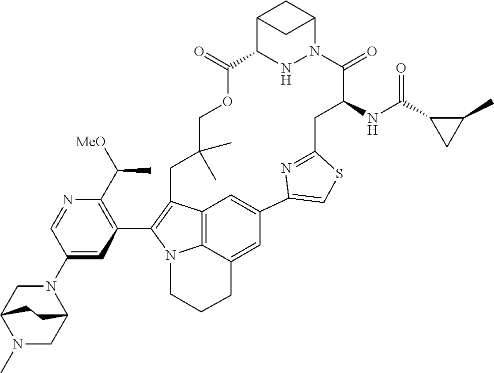

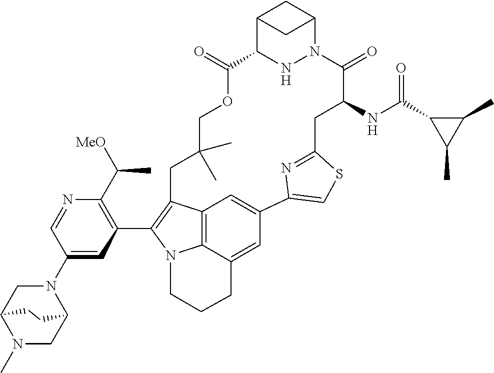

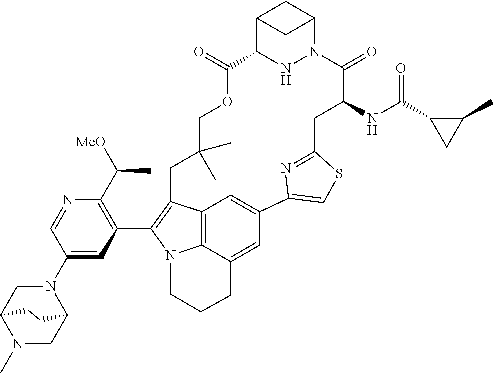

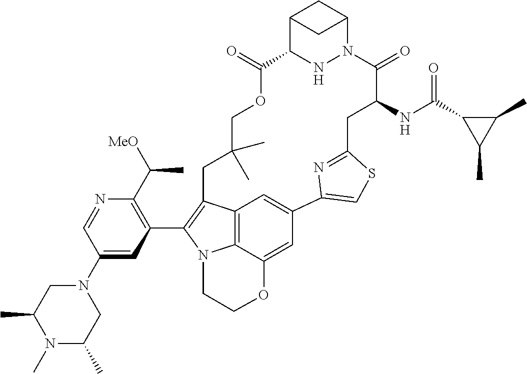

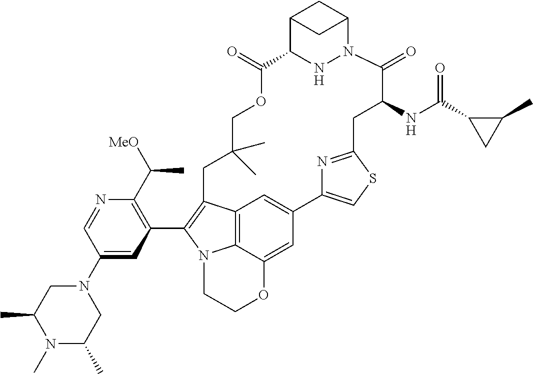

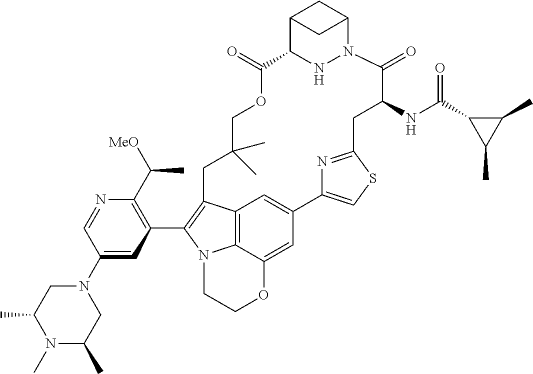

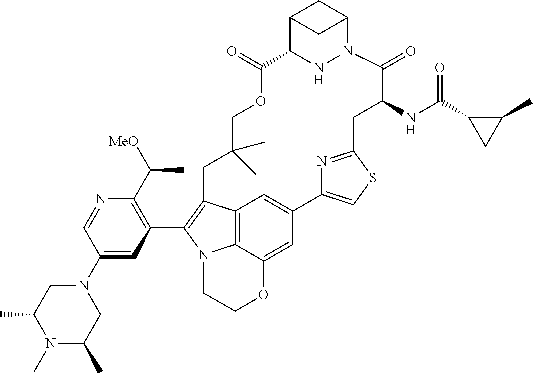

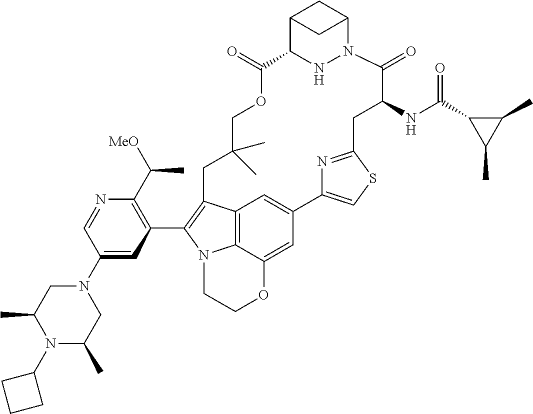

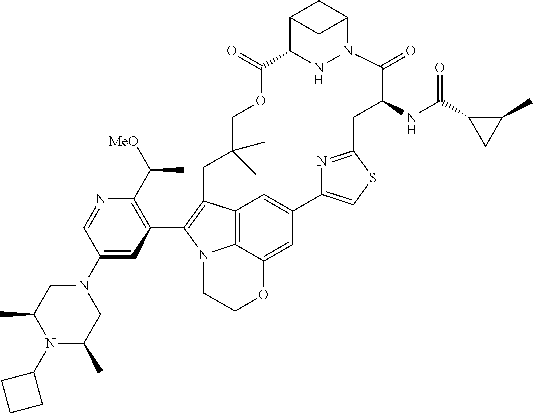

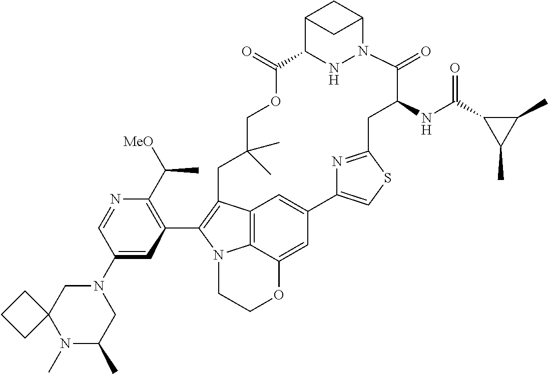

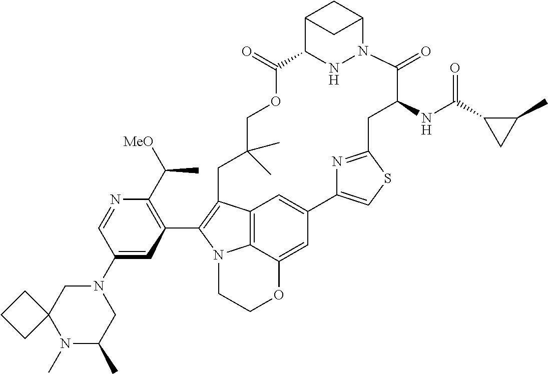

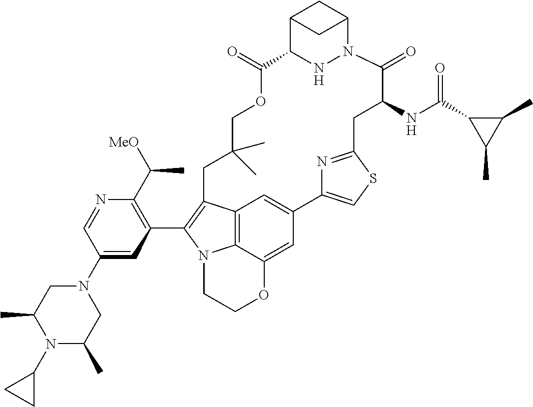

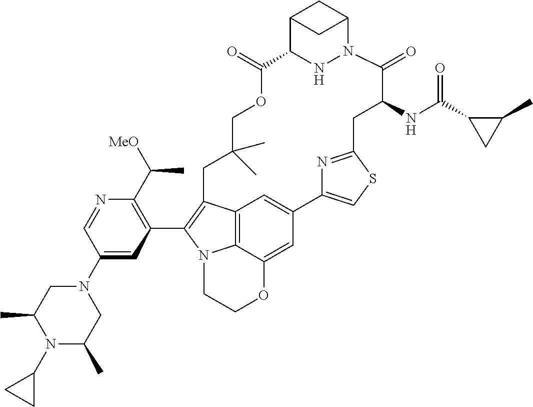

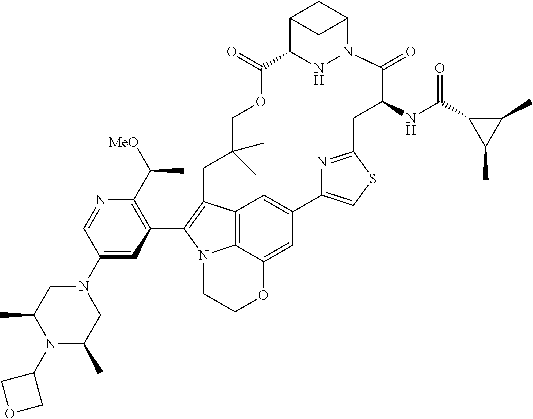

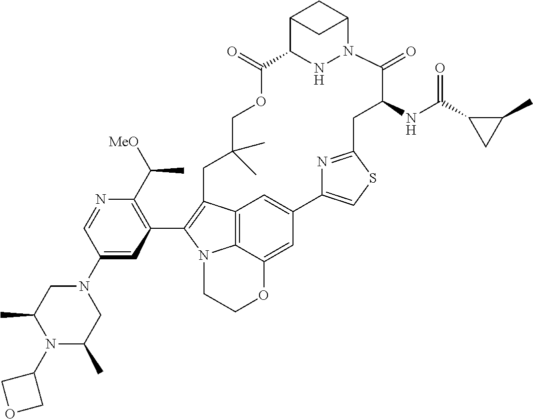

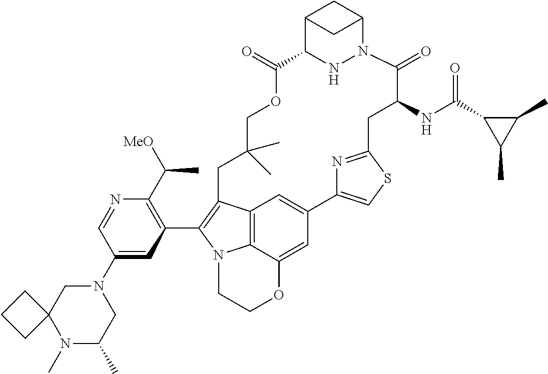

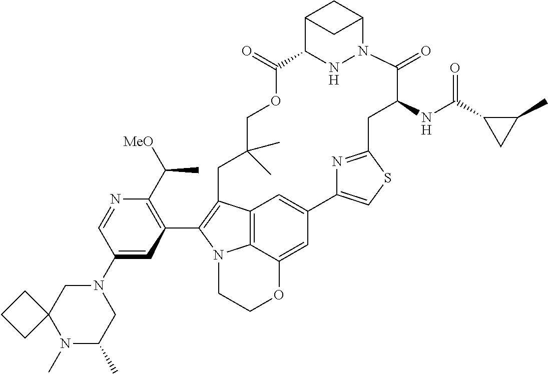

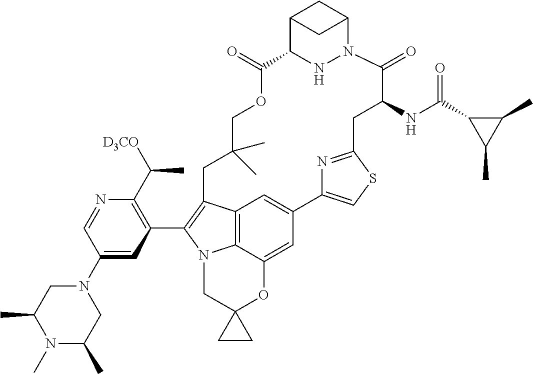

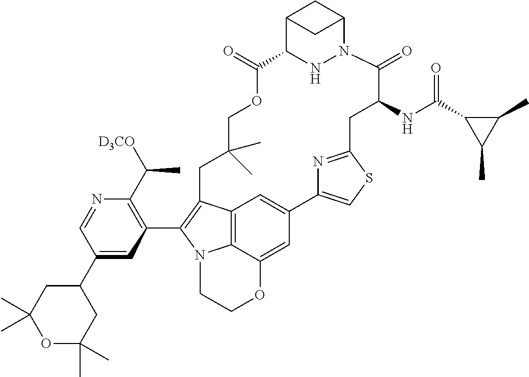

Aspect 52. A pharmaceutical composition comprising a compound of any one of Aspects 1-51, or an N-oxide, pharmaceutically acceptable salt, stereoisomer, tautomer, or deuterated analog thereof, together with a pharmaceutically acceptable excipient.

Aspect 53. A method of treating cancer in a subject in need thereof, the method comprising administering to the subject a therapeutically effective amount of a compound of any one of Aspects 1-51, or an N-oxide, pharmaceutically acceptable salt, stereoisomer, tautomer, or deuterated analog thereof, or a pharmaceutical composition of Aspect 52.

Aspect 54. The method of Aspect 53, wherein the cancer is pancreatic cancer, colorectal cancer, lung cancer, gastric cancer, esophageal cancer, ovarian cancer or uterine cancer.

Aspect 55. A method of treating RAS protein-related disorder in a subject in need thereof, the method comprising administering to the subject a therapeutically effective amount of a compound of any one of Aspects 1-51, or an N-oxide, pharmaceutically acceptable salt, stereoisomer, tautomer, or deuterated analog thereof, or a pharmaceutical composition of Aspect 52.

The compound of Formula (I) may contain asymmetric or chiral centers, and therefore, exist in different stereoisomeric forms, such as enantiomers, diastereomers, or atropisomers. All stereoisomeric forms of the compounds of Formula (I) as well as mixtures thereof, including racemic mixtures, form part of the present invention.

It should also be noted that the compound of Formula (I) can include E and Z isomers, or a mixture thereof, and cis and trans isomers or a mixture thereof.

It is also possible that the compound of Formula (I) may exist in different tautomeric forms, and all such forms are within the scope of the present invention.

In the compound of Formula (I), the atoms may exhibit their natural abundances, or one or more of the atoms may be artificially enriched in a particular isotope having the same atomic number, but an atomic mass or mass number different from the atomic mass or mass number predominantly found in nature. All suitable isotopic variations of the compound of Formula (I) are within the scope of the present invention. For example, different isotopic forms of hydrogen (H) include protium (¹H) and deuterium (²H).

### Methods for Making Compounds

The Compounds can be made using conventional organic syntheses and commercially available starting materials. By way of example and not limitation, Compounds of Formula (I) can be prepared as outlined in Schemes 1 or 2 shown below as well as in the examples set forth herein. It should be noted that one skilled in the art would know how to modify the procedures set forth in the illustrative schemes and examples to arrive at the desired products. Common protecting groups may be used to prevent certain functional groups from undergoing undesired reaction. Exemplary protecting groups are described in “Protective Groups in Organic Synthesis”, 4^{th} Edition, P. G. M. Wuts; T. W. Greene, John Wiley, 2007, and references cited therein.

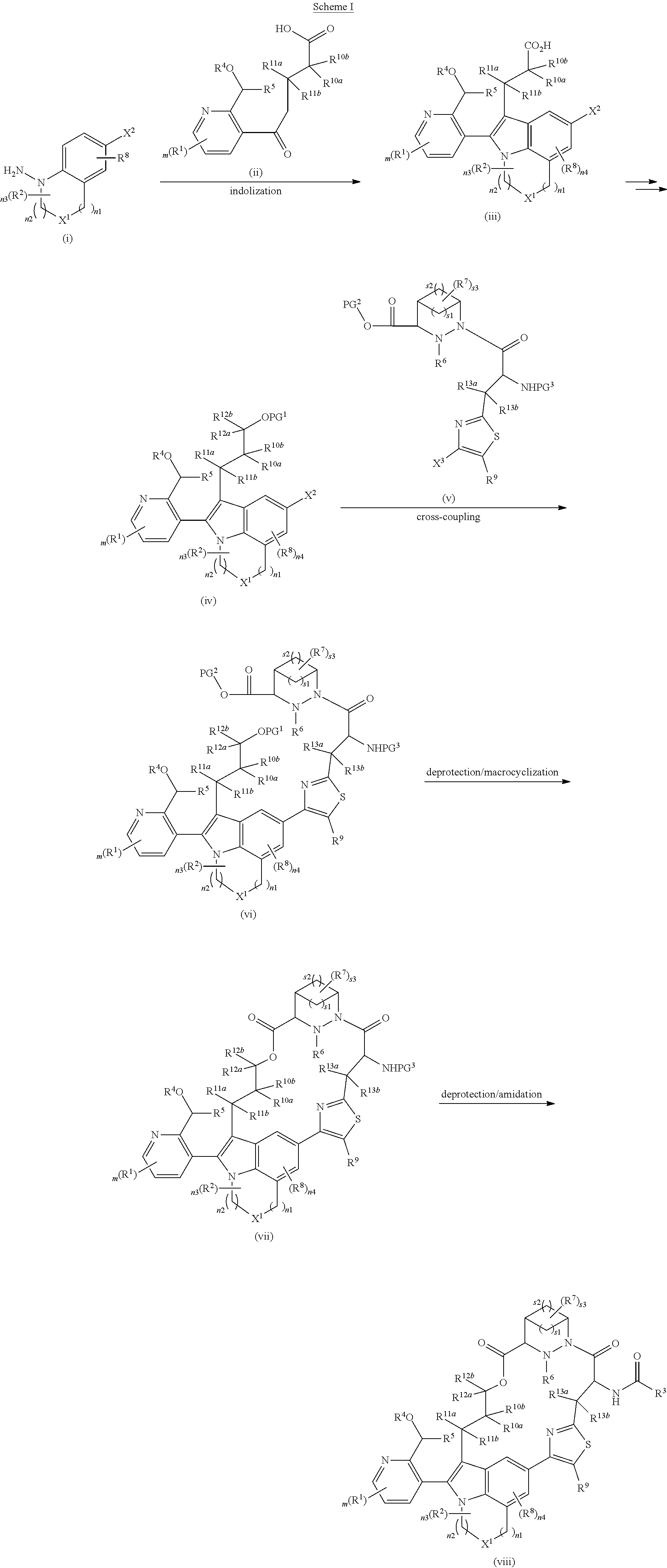

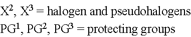

For example, compounds of Formula (I) can be formed as shown in Scheme I. Compound (i) and compound (ii) can be coupled together via indolization to give compound (iii). Compound (iii) can undergo functional group manipulations to afford compound (iv). Compound (iv) can be coupled with Compound (v) via borylation of the aryl halides and subsequent metal-catalyzed couplings to give Compound (vi). Compound (vi) can be converted into Compound (vii) via sequential deprotection and macrocyclization. Compound (vii) can undergo deprotection and amidation to give Compound (viii) [i.e., formula (I)].

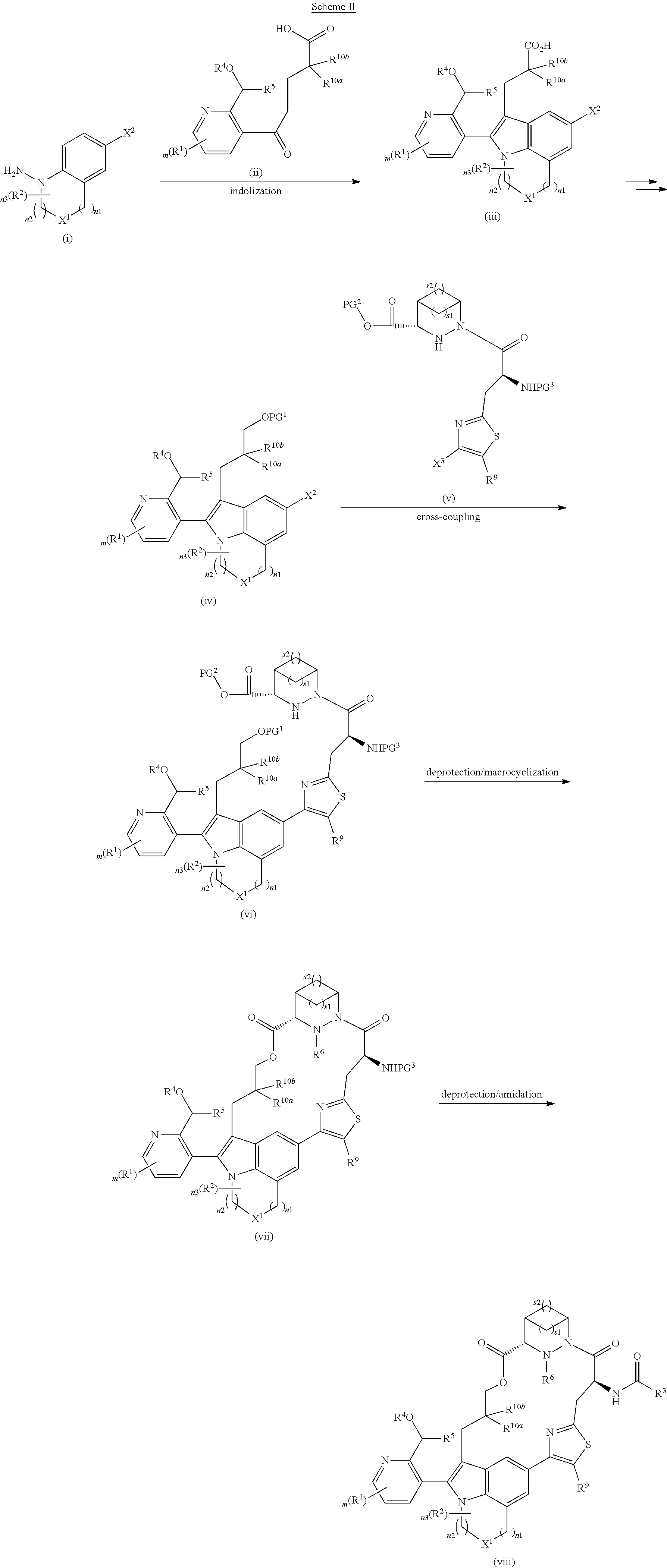

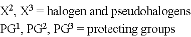

For example, compounds of Formula (I) can be formed as shown in Scheme I. Compound (i) and compound (ii) can be coupled together via indolization to give compound (iii). Compound (iii) can undergo functional group manipulations to afford compound (iv). Compound (iv) can undergo C—H functionalization to provide Compound (v). Compound (v) can be coupled with the corresponding reaction partners via metal-catalyzed coupling to give Compound (vi). Compound (vi) can be coupled with Compound (vii) via borylation of the aryl halides and subsequent metal-catalyzed couplings to give Compound (viii). Compound (viii) can be converted into Compound (ix) via sequential deprotection and macrocyclization. Compound (ix) can undergo deprotection and amidation to give Compound (x) [i.e., formula (I)].

### EXAMPLES

The examples below are intended to be purely exemplary and should not be considered to be limiting in any way. Unless otherwise specified, the experimental methods in the Examples described below are conventional methods. Unless otherwise specified, the reagents and materials are all commercially available. All solvents and chemicals employed are of analytical grade or chemical purity. Solvents are all redistilled before use. Anhydrous solvents are all prepared according to standard methods or reference methods.

¹H NMR spectra were recorded on Agilent instruments operating at 400 MHz, 500 MHz or 600 MHz. ¹HNMR spectra were obtained using CDCl₃, CD₂Cl₂, CD₃OD, D₂O, d₆-DMSO, d₆-acetone or (CD₃)₂CO as solvent and tetramethylsilane (0.00 ppm) or residual solvent (CDCl₃: 7.25 ppm; CD₃OD: 3.31 ppm; D₂O: 4.79 ppm; d₆-DMSO: 2.50 ppm; d₆-acetone: 2.05; (CD₃)₃CO: 2.05) as the reference standard. When peak multiplicities are reported, the following abbreviations are used: s (singlet), d (doublet), t (triplet), q (quartet), qn (quintuplet), sx (sextuplet), m (multiplet), br (broadened), dd (doublet of doublets), dt (doublet of triplets). Coupling constants, when given, are reported in Hertz (Hz).

LCMS-1: LC-MS spectrometer (Agilent 1260 Infinity) Detector: MWD (190-400 nm), Mass detector: 6120 SQ Mobile phase: A: water with 0.1% Formic acid, B: acetonitrile with 0.1% Formic acid Column: Poroshell 120 EC-C18, 4.6×50 mm, 2.7 pm Gradient method: Flow: 1.8 mL/min Time (min) A (%) B (%)

| Time (min) | A(%) | B(%) |
|---|---|---|
| 0.00 | 95 | 5 |
| 1.5 | 5 | 95 |
| 2.0 | 5 | 95 |
| 2.1 | 95 | 5 |
| 3.0 | 95 | 5 |

LCMS, LCMS-3: LC-MS spectrometer (Agilent 1260 Infinity II) Detector: MWD (190-400 nm), Mass detector: G6125C SQ Mobile phase: A: water with 0.1% Formic acid, B: acetonitrile with 0.1% Formic acid Column: Poroshell 120 EC-C18, 4.6×50 mm, 2.7 pm Gradient method: Flow: 1.8 mL/min Time (min) A (%) B (%)

| Time (min) | A(%) | B(%) |
|---|---|---|
| 0.00 | 95 | 5 |
| 1.5 | 5 | 95 |
| 2.0 | 5 | 95 |
| 2.1 | 95 | 5 |
| 3.0 | 95 | 5 |

LCMS-2: LC-MS spectrometer (Agilent 1290 Infinity II) Detector: MWD (190-400 nm), Mass detector: G6125C SQ Mobile phase: A: water with 0.1% Formic acid, B: acetonitrile with 0.1% Formic acid Column: Poroshell 120 EC-C18, 4.6×50 mm, 2.7 pm Gradient method: Flow: 1.2 mL/min Time (min) A (%) B (%)

| Time (min) | A(%) | B(%) |
|---|---|---|
| 0.00 | 90 | 10 |
| 1.5 | 5 | 95 |
| 2.0 | 5 | 95 |
| 2.1 | 90 | 10 |
| 3.0 | 90 | 10 |

Preparative HPLC was conducted on a column (150×21.2 mm ID, 5 pm, Gemini NXC 18; 150×19 mm ID, 5 pm, Waters Xbridge C118; 50×19 mm ID, 5 pm, Waters Xselect C118; or, 150×19 mm ID, 5 pm, Waters Sunfire C18) at a flow rate of about 10 to about 20 ml/min, injection volume about 2 ml, at room temperature and UV Detection at 214 nm and 254 nm.

In the following examples, the following abbreviations are used:

| | |
|---|---|
| NMR | nuclear magnetic resonance |
| UV | ultraviolet |
| HPLC | high performance liquid chromatography |
| prep | preparation/preparative |
| LC-MS | liquid chromatograph mass spectrometer |
| TLC | thin layer chromatography |
| PE | petroleum ether |
| Et | ethyl |
| Ac | acetyl |
| EA | ethyl acetate |
| TMS | trimethylsilyl |
| dppf | 1,1′-bis(diphenylphosphino)ferrocene |
| Me | methyl |
| DCM | dichloromethane |
| Boc | tert-butyloxycarbonyl |
| LDA | lithium diisopropylamide |
| TCFH | Chloro-N,N,N′,N′-tetramethylformamidinium |
| | hexafluorophosphate |
| NMI | N-methylimidazole |
| Bu | butyl |
| BPD | bis(pinacolato)diboron |
| DMSO | dimethyl sulfoxide |
| THF | tetrahydrofuran |
| 2-MeTHF | 2-methyltetrahydrofuran |
| Piv | pivaloyl |
| Ph | phenyl |
| DMF | N,N-dimethylformamide |
| HATU | 1-[bis (dimethylamino)methylene]-1H-1,2,3-triazolo [4,5- |
| | b]pyridinium 3-oxid hexafluorophosphate |
| DMAP | 4-dimethylaminopyridine |
| STAB | sodium triacetoxyborohydride |
| MTBE | methyl tert-butyl ether |
| Pr | propyl |
| SFC | supercritical fluid chromatography |
| TFA | trifluoroacetic acid |
| dtbpy | 4,4′-di-tert-butyl-2,2′-bipyridine |
| NMP | N-methyl-2-pyrrolidone |
| Bn | benzyl |
| Ts | tosyl |
| DIPEA | N,N-diisopropylethylamine |
| dtbpf | 1,1′-bis(di-tert-butylphosphino)ferrocene |
| Xphos- | (2-dicyclohexylphosphino-2′,4′,6′-triisopropyl-1,1′- |
| Pd-G3 | biphenyl)[2-(2′-amino-1,1′-biphenyl)]palladium(II) |
| | methanesulfonate |
| Xphos | 2-dicyclohexylphosphino-2′,4′,6′-triisopropylbiphenyl |
| FA | formic acid |
| CbzOSu | N-(benzyloxycarbonyloxy)succinimide |
| ERK | extracellular signal-regulated kinases |
| KRAS | Kirsten rat sarcoma viral oncogene homologue |
| THR | threonine |
| TYR | tyrosine |
| HTRF | homogeneous time resolved fluorescence |
| FRET | fluorescence resonance energy transfer |
| PMB | 4-methoxybenzyl |

### EXAMPLES

### Intermediate 1: 5-{2-[(1S)-1-methoxyethyl]pyridin-3-yl}-2,2-dimethyl-5-oxopentanoic Acid

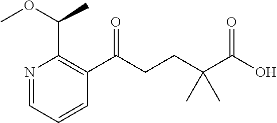 [SMILES: CO[C@@H](C)c1ncccc1C(=O)CCC(C)(C)C(=O)O]

To a mixture of i-PrMgCl (2 M in in THF, 120 mL) at −10° C. under nitrogen was added n-BuLi (2.5 M in hexanes, 80 mL, 200 mmol) dropwise. The mixture was stirred for 30 min at −10° C., and then 3-bromo-2-[(1S)-1-methoxyethyl]pyridine (43.2 g, 200 mmol) in THF (300 mL) was added dropwise at −10° C. The mixture was warmed to −5° C. and stirred for 1 h. Then, 3,3-dimethyloxane-2,6-dione (28.4 g, 200 mmol) in THF (200 mL) was added dropwise at −5° C. The mixture was warmed to 0° C. and stirred for 1.5 h. HCl (4 N in dioxane) was added at 0° C. to adjust the pH of the mixture to ˜5. The mixture was diluted with iced water (500 mL) and extracted with EA (800 mL×3). The combined organic layers were dried over Na₂SO₄, filtered and concentrated under vacuum. The residue was purified by silica gel chromatography (EA) to give intermediate 1 (13.5 g, 24%). LC-MS (M+H)⁺=280.1.

### Intermediate 2: methyl (4S)-2-[(2S)-3-(4-bromo-1,3-thiazol-2-yl)-2-{[(tert-butoxy)carbonyl]amino}propanoyl]-2,3-diazabicyclo[3.1.1]heptane-4-carboxylate

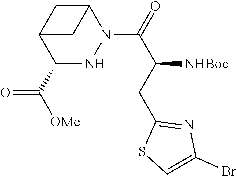 [SMILES: COC(=O)[C@H]1NN(C(=O)[C@H](Cc2nc(Br)cs2)NC(=O)OC(C)(C)C)C2CC1C2]

### Step 1: methyl (2S)-3-(4-bromo-1,3-thiazol-2-yl)-2-{[(tert-butoxy)carbonyl]amino}propanoate

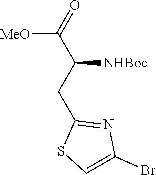 [SMILES: COC(=O)[C@H](Cc1nc(Br)cs1)NC(=O)OC(C)(C)C]

To a suspension of Zn powder (49.7 g, 760 mmol) in THF (500 mL) was added 1,2-dibromoethane (5.71 g, 30.4 mmol) dropwise. The suspension was stirred at 90° C. for 30 min and then cooled to 25° C. TMSCl (1.65 g, 15.2 mmol) was added, and the suspension was stirred for 30 min at 25° C. A solution of methyl (2R)-2-(tert-butoxycarbonylamino)-3-iodo-propanoate (50.0 g, 152 mmol) in THF (500 mL) was added dropwise and the suspension was stirred at 35° C. for 1 h. 2,4-dibromothiazole (73.8 g, 304 mmol) and Pd(PPh₃)₂Cl₂ (10.7 g, 15.2 mmol) was added to the mixture and stirred at 70° C. for 16 h. The mixture was poured into water (500 mL). The mixture was extracted with EA (200 mL×2). The combined organic phase was washed with brine (100 mL), dried over Na₂SO₄, filtered and concentrated in vacuum. The residue was purified by silica gel chromatograph (PE:EA=50:1 to 5:1) to give the title compound (5.0 g, 9%). LC-MS (M+Na)⁺=387.0/388.9.

### Step 2: (2S)-3-(4-bromo-1,3-thiazol-2-yl)-2-{[(tert-butoxy)carbonyl]amino}propanoic Acid

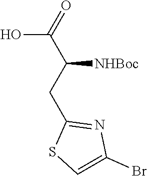 [SMILES: CC(C)(C)OC(=O)N[C@@H](Cc1nc(Br)cs1)C(=O)O]

To a solution of methyl (2S)-3-(4-bromo-1,3-thiazol-2-yl)-2-{[(tert-butoxy)carbonyl]amino}propanoate (2.0 g, 5.48 mmol) in THF (16 mL) and water (4 mL) was added LiOH·H₂O (230 mg, 5.48 mmol). The mixture was stirred at 25° C. for 1 h. The aqueous phase was extracted with DCM (30 mL×2). To the mixture was added hydrochloric acid (2 M) to adjust pH to ˜5. The mixture was poured into water (10 mL), and the aqueous phase was extracted with EA (10 mL×2). The combined organic phase was washed with brine (10 mL), dried over Na₂SO₄, filtered and concentrated in vacuum to afford the title compound (1.0 g, 52%). LC-MS (M+Na)⁺=373.0/374.9.

### Step 3: (4S)-4-benzyl-3-[2-(3-oxocyclobutyl)acetyl]-1,3-oxazolidin-2-one

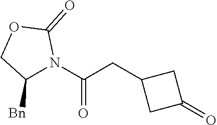 [SMILES: O=C1CC(CC(=O)N2C(=O)OC[C@@H]2Cc2ccccc2)C1]

To a mixture of 2-(3-oxocyclobutyl)acetic acid (25.0 g, 195 mmol), (S)-4-benzyloxazolidin-2-one (38.0 g, 215 mmol), Et₃N (59.0 g, 585 mmol) and DMAP (2.38 g, 19.5 mmol) in DCM (500 mL) was added 2-chloro-1-methylpyridinium iodide (84.7 g, 332 mmol) in portions at 0° C. The mixture was warmed to room temperature and stirred for 2 h. The mixture was poured into water (1 L). The organic layer was separated, and the aqueous phase was extracted with DCM (500 mL×2). The combined organic layer was washed with brine (1000 mL), dried over Na₂SO₄, filtered and concentrated under reduced pressure to give the title compound (56 g, 100%). LC-MS (M+H)⁺=288.3.

### Step 4: (4S)-4-benzyl-3-[2-(3-hydroxycyclobutyl)acetyl]-1,3-oxazolidin-2-one

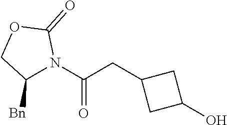 [SMILES: O=C(CC1CC(O)C1)N1C(=O)OC[C@@H]1Cc1ccccc1]

To a mixture of (4S)-4-benzyl-3-[2-(3-oxocyclobutyl)acetyl]-1,3-oxazolidin-2-one (56 g, 195 mmol) and AcOH (23.4 g, 390 mmol) in THF (500 mL) was added NaBH₄ (7.41 g, 195 mmol) in portions at 0° C. The mixture was stirred at 0° C. for 2 h and saturated NH₄Cl (300 mL) was added slowly. The mixture was extracted with EA (300 mL×2). The combined organic layer was washed with saturated NaHCO₃ (300 mL×2), brine (500 mL), dried over Na₂SO₄, filtered and concentrated under reduced pressure to give the title compound (56.4 g, 100%). LC-MS (M+H)⁺=290.3.

### Step 5: 3-{2-[(4S)-4-benzyl-2-oxo-1,3-oxazolidin-3-yl]-2-oxoethyl}cyclobutyl 4-methylbenzene-1-sulfonate

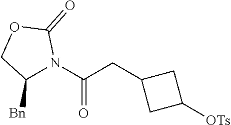 [SMILES: Cc1ccc(S(=O)(=O)OC2CC(CC(=O)N3C(=O)OC[C@@H]3Cc3ccccc3)C2)cc1]

To a mixture of (4S)-4-benzyl-3-[2-(3-hydroxycyclobutyl)acetyl]-1,3-oxazolidin-2-one (56.0 g, 195 mmol), DIPEA (37.7 g, 292 mmol) and DMAP (23.8 g, 195 mmol) in DCM (500 mL) was added tosyl chloride (40.9 g, 214 mmol) in portions at 0° C. The mixture was stirred at room temperature for 2 h. The mixture was poured into water (1 L). The organic layer was separated and the aqueous layer was extracted with DCM (500 mL×2). The combined organic layer was washed with brine (1 L), dried over Na₂SO₄, filtered and concentrated under reduced pressure. The residue was purified by silica gel chromatograph (PE:EA=20:1 to 3:1) to give the title compound (76 g, 88%). LC-MS (M+H)⁺=444.3.

### Step 6: (4S)-4-benzyl-3-[2-(3-bromocyclobutyl)acetyl]-1,3-oxazolidin-2-one

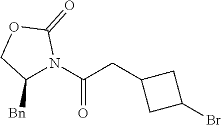 [SMILES: O=C(CC1CC(Br)C1)N1C(=O)OC[C@@H]1Cc1ccccc1]

A mixture of 3-{2-[(4S)-4-benzyl-2-oxo-1,3-oxazolidin-3-yl]-2-oxoethyl}cyclobutyl 4-methylbenzene-1-sulfonate (76.0 g, 172 mmol) and LiBr (29.8 g, 343 mmol) in NMP (760 mL) was stirred at 90° C. for 13 h. The mixture was cooled to room temperature and poured into water (1 L). The mixture was extracted with EA (500 mL×2). The combined organic layer was washed with brine (1 L), dried over Na₂SO₄, filtered and concentrated under reduced pressure. The residue was purified by silica gel chromatograph (PE:EA=20:1 to 4:1) to give the title compound (48.1 g, 80%). LC-MS (M+H)⁺=352.3.

### Step 7: (4S)-2,3-bis[(tert-butoxy)carbonyl]-2,3-diazabicyclo[3,1,1]heptane-4-carboxylic Acid

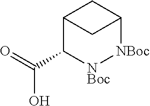 [SMILES: CC(C)(C)OC(=O)N1C2CC(C2)[C@@H](C(=O)O)N1C(=O)OC(C)(C)C]

To a mixture of (4S)-4-benzyl-3-[2-(3-bromocyclobutyl)acetyl]-1,3-oxazolidin-2-one (48.1 g, 136 mmol) in THF (400 mL) was added LDA (2.0 M in THF/hexane, 88.5 mL, 177 mmol) dropwise at −78° C. under nitrogen. The mixture was stirred at −78° C. for 30 min followed by addition of di-tert-butyl azodicarboxylate (37.7 g, 164 mmol) in THF (100 mL). The mixture was stirred at −78° C. for 30 min followed by slow addition of 1,3-dimethyl-1,3-diazinan-2-one (522 g, 4.08 mol). The mixture was slowly warmed to room temperature and stirred for 13 h. Water (100 mL) and LiOH·H₂O (17.1 g, 408 mmol) was added to the reaction mixture. The mixture was stirred at room temperature for 1 h and concentrated under reduced pressure. The residue was treated with brine (200 mL) and washed with MTBE (200 mL×2). The organic layer was discarded, and the pH of aqueous phase was adjusted to ˜5 with 1 N hydrochloric acid at 0° C. The mixture was extracted with EA (200 mL×2). The combined organic layer was washed with brine (1000 mL), dried over Na₂SO₄, filtered and concentrated under reduced pressure. The residue was purified by silica gel chromatograph (PE:EA=20:1 to 3:1) to give the title compound (26.0 g, 56%). LC-MS (M+H)⁺=343.3.

### Step 8: 2,3-di-tert-butyl 4-methyl (4S)-2,3-diazabicyclo[3.1.1]heptane-2,3,4-tricarboxylate

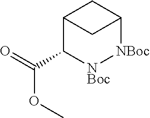 [SMILES: COC(=O)[C@@H]1C2CC(C2)N(C(=O)OC(C)(C)C)N1C(=O)OC(C)(C)C]

To a mixture of (4S)-2,3-bis[(tert-butoxy)carbonyl]-2,3-diazabicyclo[3.1.1]heptane-4-carboxylic acid (26 g, 76 mmol) in MeOH (150 mL) was added (trimethylsilyl)diazomethane (2.0 M in hexanes, 190 mL, 380 mmol) dropwise with vigorous stirring. The mixture was stirred at 25° C. for 1 h followed by addition of AcOH (1 mL). The mixture was concentrated under reduced pressure. The residue was purified by silica gel chromatography (PE:EA=20:1 to 3:1) to give the title compound (5.6 g, 21%). LC-MS (M+H)⁺=357.3.

### Step 9: methyl (4S)-2,3-diazabicyclo[3.1.1]heptane-4-carboxylate

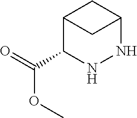

To mixture of 2,3-di-tert-butyl 4-methyl (4S)-2,3-diazabicyclo[3.1.1]heptane-2,3,4-tricarboxylate (5.6 g, 15.7 mmol) in dioxane (30 mL) was added HCl (4 M in dioxane, 30 mL). The mixture was stirred at room temperature for overnight and concentrated under vacuum. The residue was treated with DCM (20 mL) and washed with saturated NaHCO₃ (10 mL). The organic layer was dried over Na₂SO₄, filtered and concentrated under vacuum to give the title compound (2.45 g, 100%). LC-MS (M+H)⁺=157.3.

### Step 10: methyl (4S)-2-[(2S)-3-(4-bromo-1,3-thiazol-2-yl)-2-{[(tert-butoxy)carbonyl]amino}propanoyl]-2,3-diazabicyclo[3.1.1]heptane-4-carboxylate

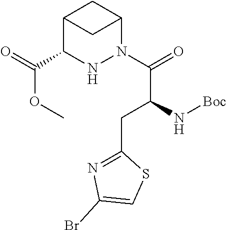 [SMILES: COC(=O)[C@H]1NN(C(=O)[C@H](Cc2nc(Br)cs2)NC(=O)OC(C)(C)C)C2CC1C2]

A mixture of methyl (4S)-2,3-diazabicyclo[3.1.1]heptane-4-carboxylate (2.45 g, 15.7 mmol), (2S)-3-(4-bromo-1,3-thiazol-2-yl)-2-{[(tert-butoxy)carbonyl]amino}propanoic acid (5.51 g, 15.7 mmol), HATU (11.93 g, 31.4 mmol) and DIPEA (10.13 g, 78.5 mmol) in DMF (200 mL) was stirred at room temperature for 2 h under nitrogen. The residue was diluted with EA (200 mL), washed with brine (200 mL×2), dried over Na₂SO₄, filtered and concentrated under vacuum. The residue was purified by silica gel chromatography (PE:EA=4:1 to 1:1) then by SFC to give intermediate 2 (3.2 g, 42%). ¹H NMR (Chloroform-d) δ 7.11 (s, 1H), 5.80-5.24 (m, 3H), 4.68 (t, J=4.8 Hz, 1H), 4.39-4.08 (m, 1H), 3.77 (s, 3H), 3.53 (dd, J=14.6, 5.5 Hz, 1H), 3.45 (dd, J=14.6, 5.1 Hz, 1H), 2.70 (q, J=6.0 Hz, 1H), 2.44 (dtd, J=10.6, 5.2, 2.9 Hz, 1H), 2.34 (dt, J=10.7, 4.9 Hz, 1H), 2.00-1.90 (m, 1H), 1.82 (t, J=9.7 Hz, 1H), 1.44 (s, 9H). LC-MS (M+H)⁺=489.3/491.3.

### Example 1: (1R,2R,3S)—N-[(7′S,13′S)-20′-{2-[(1S)-1-methoxyethyl]-5-[(3R,5S)-3,4,5-trimethylpiperazin-1-yl]pyridin-3-yl}-17′,17′-dimethyl-8′,14′-dioxo-15′,24′-dioxa-4′-thia-9′,21′,30′,32′-tetraazaspiro[cyclopropane-1,23′-heptacyclo[23.3.1.1^{2,5}.1^{9,13}.1^{10,12}.0^{19,27}.0^{21,26}]dotriacontane]-1′(28′),2′,5′(32′),19′,25′(29′),26′-hexaen-7′-yl]-2,3-dimethylcyclopropane-1-carboxamide

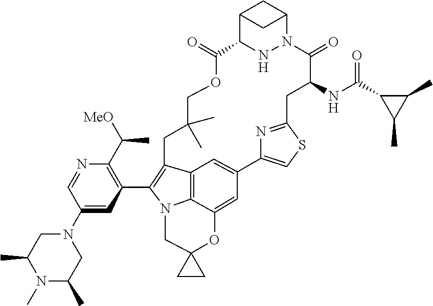

### Step 1: ethyl 1-(5-bromo-2-nitrophenoxy)cyclopropane-1-carboxylate

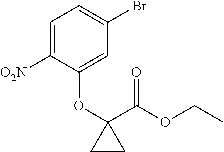 [SMILES: CCOC(=O)C1(Oc2cc(Br)ccc2[N+](=O)[O-])CC1]

To a solution of ethyl 1-hydroxycyclopropane-1-carboxylate (5.9 g, 45 mmol) in THF (150 mL) added NaH (60%, 1.8 g, 45 mmol) at 0° C. under nitrogen. The mixture was stirred at room temperature for 15 min followed by addition of 4-bromo-2-fluoro-1-nitrobenzene (8.3 g, 37.7 mmol). The mixture was stirred at room temperature overnight. The mixture was added to saturated solution of NH₄Cl (100 mL) and extracted with EA (120 mL×3). The combined organic layer was washed with brine (100 mL), dried over Na₂SO₄, filtered and concentrated under reduced pressure. The residue was dissolved in EA/PE (1:1, 140 mL) and filtered through a short silica gel pad (50 mL). The silica gel was further eluted with EA/PE (1:1, 600 mL). The filtrate was concentrated under reduced pressure to give the title compound (14.0 g, 94%). ¹H NMR (400 MHz, DMSO-d6) δ 7.84 (d, J=8.4 Hz, 1H), 7.43-7.31 (m, 2H), 4.10 (q, J=7.1 Hz, 2H), 1.60 (dd, J=8.7, 5.4 Hz, 2H), 1.41 (dd, J=8.6, 5.3 Hz, 2H), 1.07 (t, J=7.1 Hz, 3H). LC-MS (M+H)⁺=330.1.

### Step 2: 7-bromo-3,4-dihydrospiro[1,4-benzoxazine-2,1′-cyclopropan]-3-one

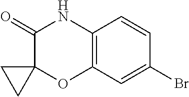 [SMILES: O=C1Nc2ccc(Br)cc2OC12CC2]

To a mixture of ethyl 1-(5-bromo-2-nitrophenoxy)cyclopropane-1-carboxylate (4.1 g, 12.5 mmol) in DMF (20 mL) was added tetrahydroxydiboron (4.5 g, 50 mmol) and the mixture was stirred at 100° C. for overnight. The mixture was cooled to room temperature and water (120 mL) was added. The mixture was aged with stirring for 30 min, and the solid was collected by filtration. The solid was dried under vacuum to give the title compound (2.85 g, 84%). ¹H NMR (400 MHz, DMSO-d6) δ 10.90 (s, 1H), 7.18 (d, J=8.4 Hz, 1H), 7.10 (s, 1H), 6.86 (d, J=8.4 Hz, 1H), 1.28-1.24 (m, 2H), 1.21-1.18 (m, 2H). LC-MS (M+H)⁺=254.0.

### Step 3: 7-bromo-3,4-dihydrospiro[1,4-benzoxazine-2,1′-cyclopropane]

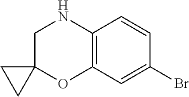 [SMILES: Brc1ccc2c(c1)OC1(CC1)CN2]

To a solution of 7-bromo-3,4-dihydrospiro[1,4-benzoxazine-2,1′-cyclopropan]-3-one (2.85 g, 11.2 mmol) in THF (40 mL) at 0° C. was added borane (1.0 M in THF, 22.4 mL, 22.4 mmol) under nitrogen. The mixture was stirred at 70° C. for 2 h and cooled to room temperature. The mixture was slowly added to aqueous NaOH (1 M, 100 mL) and extracted with EA (100 mL×3). The combined organic layer was washed with brine (100 mL), dried over Na₂SO₄, filtered and concentrated under reduced pressure. The residue was purified by silica gel chromatograph (EA/PE=1/10) to give the title compound (2.2 g, 82%). ¹H NMR (400 MHz, DMSO-d6) δ 6.78 (dd, J=8.4, 2.2 Hz, 1H), 6.68 (d, J=2.2 Hz, 1H), 6.50 (d, J=8.4 Hz, 1H), 6.07 (s, 1H), 3.17 (d, J=2.3 Hz, 2H), 0.85-0.82 (m, 2H), 0.66-0.63 (m, 2H). LC-MS (M+H)⁺=240.0.

### Step 4: 7-bromo-3,4-dihydrospiro[1,4-benzoxazine-2,1′-cyclopropan]-4-amine

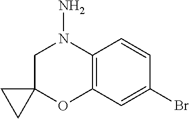 [SMILES: NN1CC2(CC2)Oc2cc(Br)ccc21]

To a solution of 7-bromo-3,4-dihydrospiro[1,4-benzoxazine-2,1′-cyclopropane](2.2 g 9.2 mmol) in EtOH (20 mL) was added a solution of NaNO₂ (950 mg, 13.7 mmol) in water (7 mL) at 0° C. Concentrated hydrochloric acid (1.3 mL) was added dropwise and the mixture was stirred at 0° C. for 1 h. A solution of NaOH (3.7 g, 92 mmol) and Na₂S₂O₄ (4.8 g, 27.6 mmol) in water (20 mL) was added, and the mixture was stirred at 90° C. for 1 h. The mixture was cooled to room temperature and diluted with water (120 mL). The mixture was extracted with EA (100 mL×3). The combined organic layer was washed with brine (100 mL), dried over Na₂SO₄, filtered and concentrated under reduced pressure. The residue was purified by silica gel chromatograph (EA/PE=1/10) to give the title compound (1.0 g, 43%). ¹H NMR (400 MHz, DMSO-d6) δ 7.08 (d, J=8.7 Hz, 1H), 6.91 (dd, J=8.7, 2.1 Hz, 1H), 6.69 (d, J=2.1 Hz, 1H), 4.38 (s, 2H), 3.23 (s, 2H), 0.87 (t, J=6.3 Hz, 2H), 0.70 (t, J=6.3 Hz, 2H). LC-MS (M-NH₂)⁺=238.0.

### Step 5: 3-(10′-bromo-3′-{2-[(1S)-1-methoxyethyl]pyridin-3-yl}-7′-oxa-4′-azaspiro[cyclopropane-1,6′-tricyclo[6.3.1.0^{4,12}]dodecane]-1′(12′),2′,8′,10′-tetraen-2′-yl)-2,2-dimethylpropanoic Acid

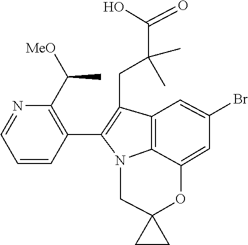 [SMILES: CO[C@@H](C)c1ncccc1-c1c(CC(C)(C)C(=O)O)c2cc(Br)cc3c2n1CC1(CC1)O3]

A mixture of 7-bromo-3,4-dihydrospiro[1,4-benzoxazine-2,1′-cyclopropan]-4-amine (1.0 g, 3.9 mmol), 5-{2-[(1S)-1-methoxyethyl]pyridin-3-yl}-2,2-dimethyl-5-oxopentanoic acid (1.1 g, 3.9 mmol) and p-toluenesulfonic acid (1.5 g, 7.9 mmol) in toluene (30 mL) was stirred at 110° C. overnight under nitrogen. The mixture was cooled to room temperature and concentrated under reduced pressure. The residue was dissolved in DCM (80 mL) washed with saturated NaHCO₃ (40 mL) and brine (40 mL), dried over Na₂SO₄, filtered and concentrated under reduced pressure to give the title compound (1.9 g, 84%). LC-MS (M+H)⁺=499.3/501.2.

### Step 6: ethyl 3-(10′-bromo-3′-{2-[(1S)-1-methoxyethyl]pyridin-3-yl}-7′-oxa-4′-azaspiro[cyclopropane-1,6′-tricyclo[6.3.1.0^{4,12}]dodecane]-1′(12′),2′,8′,10′-tetraen-2′-yl)-2,2-dimethylpropanoate

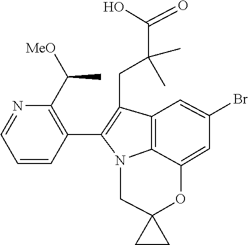 [SMILES: CO[C@@H](C)c1ncccc1-c1c(CC(C)(C)C(=O)O)c2cc(Br)cc3c2n1CC1(CC1)O3]

A mixture of 3-(10′-bromo-3′-{2-[(1S)-1-methoxyethyl]pyridin-3-yl}-7′-oxa-4′-azaspiro[cyclopropane-1,6′-tricyclo[6.3.1.0^{4,12}]dodecane]-1′(12′),2′,8′,10′-tetraen-2′-yl)-2,2-dimethylpropanoic acid (1.9 g, 3.8 mmol), iodoethane (711 mg, 4.56 mmol) and Cs₂CO₃ (1.48 g, 4.56 mmol) in DMF (6 mL) was stirred at room temperature for 1 h. The mixture was diluted with water (60 mL) and extracted with EA (50 mL×3). The combined organic layer was washed with brine (50 mL), dried over Na₂SO₄, filtered and concentrated under reduced pressure. The residue was purified by silica gel chromatograph (EA/PE=3/2) to give the title compound (1.75 g, 87%). LC-MS (M+H)⁺=527.3.

### Step 7: 3-(10′-bromo-3′-{2-[(1S)-1-methoxyethyl]pyridin-3-yl}-7′-oxa-4′-azaspiro[cyclopropane-1,6′-tricyclo[6.3.1.0^{4,12}]dodecane]-1′(12′),2′,8′,10′-tetraen-2′-yl)-2,2-dimethylpropan-1-ol

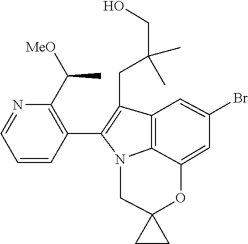 [SMILES: CO[C@@H](C)c1ncccc1-c1c(CC(C)(C)CO)c2cc(Br)cc3c2n1CC1(CC1)O3]

To a solution of ethyl 3-(10′-bromo-3′-{2-[(1S)-1-methoxyethyl]pyridin-3-yl}-7′-oxa-4′-azaspiro[cyclopropane-1,6′-tricyclo[6.3.1.0^{4,12}]dodecane]-1′(12′),2′,8′,10′-tetraen-2′-yl)-2,2-dimethylpropanoate (1.75 g, 3.32 mmol) in THF (40 mL) was added LiBH₄ (219 mg, 10.0 mmol) at 0° C. under nitrogen. The mixture was stirred at 60° C. overnight and then cooled to room temperature. The mixture was cooled to 0° C. and a saturated solution of NH₄Cl (50 mL) was added until the cease of bubbling. The mixture was extracted with EA (50 mL×3). The combined organic layer was washed with brine (50 mL), dried over Na₂SO₄, filtered and concentrated under reduced pressure to give the tile compound (1.6 g, 99%). LC-MS (M+H)⁺=485.4.

### Step 8: 3-(10′-bromo-3′-{2-[(1S)-1-methoxyethyl]pyridin-3-yl}-7′-oxa-4′-azaspiro[cyclopropane-1,6′-tricyclo[6.3.1.0^{4,12}]dodecane]-1′(12′),2′,8′,10′-tetraen-2′-yl)-2,2-dimethylpropyl Acetate

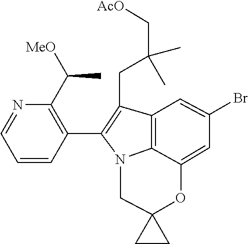 [SMILES: CO[C@@H](C)c1ncccc1-c1c(CC(C)(C)COC(C)=O)c2cc(Br)cc3c2n1CC1(CC1)O3]

To a solution of 3-(10′-bromo-3′-{2-[(1S)-1-methoxyethyl]pyridin-3-yl}-7′-oxa-4′-azaspiro[cyclopropane-1,6′-tricyclo[6.3.1.0^{4,12}]dodecane]-1′(12′),2′,8′,10′-tetraen-2′-yl)-2,2-dimethylpropan-1-ol (1.6 g, 3.3 mmol), DMAP (40 mg, 0.33 mmol) and Et₃N (670 mg, 6.6 mmol) in DCM (50 mL) at 0° C. was added Ac₂O (372 mg, 3.65 mmol) and the mixture was stirred at room temperature for 1 h. The mixture was diluted with DCM (50 mL) and successively washed with water (50 mL) and brine (50 mL). The organic layer was dried over Na₂SO₄, filtered and concentrated under reduced pressure. The residue was purified by silica gel chromatograph (DCM/MeOH=20/1) to give the title compound (1.6 g, 92%). LC-MS (M+H)⁺=527.3.

### Step 9: (5-{2′-[3-(acetyloxy)-2,2-dimethylpropyl]-10′-bromo-7′-oxa-4′-azaspiro[cyclopropane-1,6′-tricyclo[6.3.1.0^{4,12}]dodecane]-1′(12′),2′,8′,10′-tetraen-3′-yl}-6-[(1S)-1-methoxyethyl]pyridin-3-yl)boronic Acid

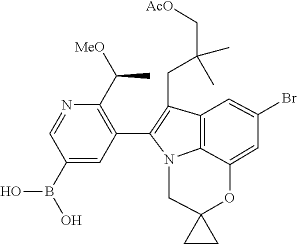 [SMILES: CO[C@@H](C)c1ncc(B(O)O)cc1-c1c(CC(C)(C)COC(C)=O)c2cc(Br)cc3c2n1CC1(CC1)O3]

To a mixture of 3-(10′-bromo-3′-{2-[(1S)-1-methoxyethyl]pyridin-3-yl}-7′-oxa-4′-azaspiro[cyclopropane-1,6′-tricyclo[6.3.1.0^{4,12}]dodecane]-1′(12′),2′,8′,10′-tetraen-2′-yl)-2,2-dimethylpropyl acetate (1.6 g, 3.04 mmol) and pinacolborane (1.95 g, 15.2 mmol) in anhydrous THF (30 mL) under nitrogen was added chloro(1,5-cyclooctadiene)iridium(I) dimer (199 mg, 0.30 mmol) and 4,4′-di-tert-butyl-2,2′-bipyridine (161 mg, 0.60 mmol). The mixture was stirred at 80° C. in a sealed tube overnight and then cooled to room temperature. The mixture was concentrated under reduced pressure. The residue was dissolved in DCM (150 mL), successively washed with water (50 mL) and brine (50 mL). The organic phase was dried over Na₂SO₄, filtered and concentrated under reduced pressure. The residue was purified by C18 chromatograph [MeCN/(0.1% TFA in water)=3:2] to give the title compound (1.4 g, 81%). LC-MS (M+H)⁺=571.3.

### Step 10: 3-(10′-bromo-3′-{2-[(1S)-1-methoxyethyl]-5-[(3R,5S)-3,4,5-trimethylpiperazin-1-yl]pyridin-3-yl}-7′-oxa-4′-azaspiro[cyclopropane-1,6′-tricyclo[6.3.1.0^{4,12}]dodecane]-1′(12′),2′,8′,10′-tetraen-2′-yl)-2,2-dimethylpropyl Acetate

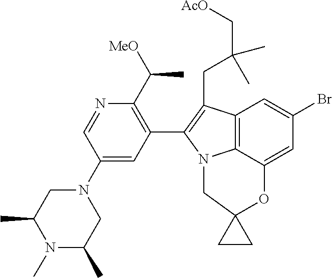 [SMILES: CO[C@@H](C)c1ncc(N2C[C@@H](C)N(C)[C@@H](C)C2)cc1-c1c(CC(C)(C)COC(C)=O)c2cc(Br)cc3c2n1CC1(CC1)O3]

A mixture of (5-{2′-[3-(acetyloxy)-2,2-dimethylpropyl]-10′-bromo-7′-oxa-4′-azaspiro[cyclopropane-1,6′-tricyclo[6.3.1.0^{4,12}]dodecane]-1′(12′),2′,8′,10′-tetraen-3′-yl}-6-[(1S)-1-methoxyethyl]pyridin-3-yl)boronic acid (700 mg, 1.23 mmol), (2R,6S)-1,2,6-trimethylpiperazine (785 mg, 6.13 mmol), Cu(OAc)₂ (448 mg, 2.46 mmol) and Et₃N (373 mg, 3.69 mmol) in MeCN (15 mL) was stirred at room temperature overnight under oxygen. The mixture was concentrated under reduced pressure. The residue was treated with DCM (50 mL) and water (50 mL), and solid was filtered off. The organic layer was separated, and the aqueous layer was extracted with DCM (50 mL×3). The combined organic layer was washed with brine (50 mL), dried over Na₂SO₄, filtered and concentrated under reduced pressure. The residue was purified by silica gel chromatograph (DCM/MeOH=20/1) to give the title compound (400 mg, 50%). LC-MS (M+H)⁺=653.4.

### Step 11: 3-(3′-{2-[(1S)-1-methoxyethyl]-5-[(3R,5S)-3,4,5-trimethylpiperazin-1-yl]pyridin-3-yl}-10′-(4,4,5,5-tetramethyl-1,3,2-dioxaborolan-2-yl)-7′-oxa-4′-azaspiro[cyclopropane-1,6′-tricyclo[6.3.1.0^{4,12}]dodecane]-1′(12′),2′,8′,10′-tetraen-2′-yl)-2,2-dimethylpropyl Acetate

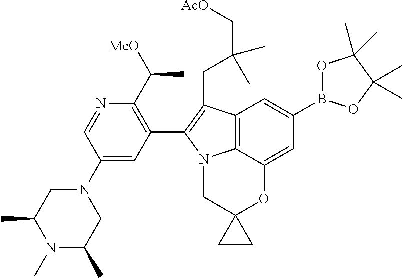

To a mixture of 3-(10′-bromo-3′-{2-[(1S)-1-methoxyethyl]-5-[(3R,5S)-3,4,5-trimethylpiperazin-1-yl]pyridin-3-yl}-7′-oxa-4′-azaspiro[cyclopropane-1,6′-tricyclo[6.3.1.0^{4,12}]dodecane]-1′(12′),2′,8′,10′-tetraen-2′-yl)-2,2-dimethylpropyl acetate (400 mg, 0.61 mmol) and BPD (233 mg, 0.92 mmol) in dioxane (15 mL) was added Pd(dppf)Cl₂ (45 mg, 0.061 mmol) and KOAc (179 mg, 1.83 mmol) under nitrogen. The mixture was stirred at 100° C. for 3 h and cooled to room temperature. Solid was filtered off and the filtrate was concentrated under reduced pressure to give the title compound in crude form which was used in step 12 without further purification. LC-MS (M+H)⁺=701.6.

### Step 12: methyl (4S)-2-[(2S)-3-(4-{2′-[3-(acetyloxy)-2,2-dimethylpropyl]-3′-{2-[(1S)-1-methoxyethyl]-5-[(3R,5S)-3,4,5-trimethylpiperazin-1-yl]pyridin-3-yl}-7′-oxa-4′-azaspiro[cyclopropane-1,6′-tricyclo[6.3.1.0^{4,12}]dodecane]-1′(12′),2′,8′,10′-tetraen-10′-yl}-1,3-thiazol-2-yl)-2-{[(tert-butoxy)carbonyl]amino}propanoyl]-2,3-diazabicyclo[3.1.1]heptane-4-carboxylate

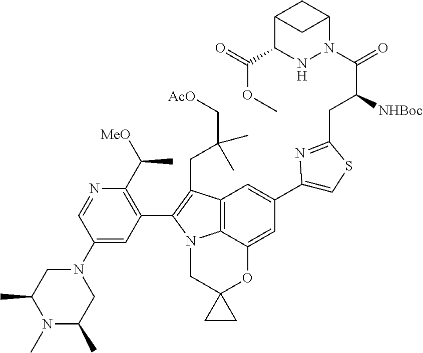 [SMILES: COC(=O)[C@H]1NN(C(=O)[C@H](Cc2nc(-c3cc4c5c(c3)c(CC(C)(C)COC(C)=O)c(-c3cc(N6C[C@@H](C)N(C)[C@@H](C)C6)cnc3[C@H](C)OC)n5CC3(CC3)O4)cs2)NC(=O)OC(C)(C)C)C2CC1C2]

A mixture of 3-(3′-{2-[(1S)-1-methoxyethyl]-5-[(3R,5S)-3,4,5-trimethylpiperazin-1-yl]pyridin-3-yl}-10′-(4,4,5,5-tetramethyl-1,3,2-dioxaborolan-2-yl)-7′-oxa-4′-azaspiro[cyclopropane-1,6′-tricyclo[6.3.1.0^{4,12}]dodecane]-1′(12′),2′,8′,10′-tetraen-2′-yl)-2,2-dimethylpropyl acetate (crude from step 11), methyl (4S)-2-[(2S)-3-(4-bromo-1,3-thiazol-2-yl)-2-{[(tert-butoxy)carbonyl]amino}propanoyl]-2,3-diazabicyclo[3.1.1]heptane-4-carboxylate (120 mg, 0.25 mmol), Pd(dtbpf)Cl₂ (45 mg, 0.061 mmol) and K₃PO₄ (179 mg, 1.83 mmol) in dioxane (25 mL) and water (4 mL) was stirred at 90° C. under nitrogen overnight. The mixture was cooled to room temperature and concentrated under reduced pressure. The residue was treated with DCM (50 mL) and water (50 mL). The organic layer was separated, and the aqueous layer was extracted with DCM (50 mL×3). The combined organic layer was washed with brine (50 mL), dried over Na₂SO₄, filtered and concentrated under reduced pressure. The residue was purified by prep-TLC (DCM/MeOH=12/1) to give the title compound (50 mg, 8% over two steps). LC-MS (M+H)⁺=983.6.

### Step 13: (4S)-2-[(2S)-2-{[(tert-butoxy)carbonyl]amino}-3-{4-[2′-(3-hydroxy-2,2-dimethylpropyl)-3′-{2-[(1S)-1-methoxyethyl]-5-[(3R,5S)-3,4,5-trimethylpiperazin-1-yl]pyridin-3-yl}-7′-oxa-4′-azaspiro[cyclopropane-1,6′-tricyclo[6.3.1.0^{4,12}]dodecane]-1′(12′),2′,8′,10′-tetraen-10′-yl]-1,3-thiazol-2-yl}propanoyl]-2,3-diazabicyclo[3.1.1]heptane-4-carboxylic Acid

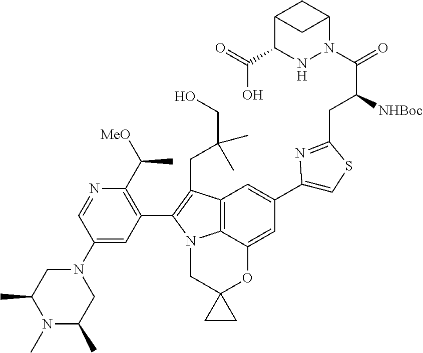 [SMILES: CO[C@@H](C)c1ncc(N2C[C@@H](C)N(C)[C@@H](C)C2)cc1-c1c(CC(C)(C)CO)c2cc(-c3csc(C[C@H](NC(=O)OC(C)(C)C)C(=O)N4N[C@H](C(=O)O)C5CC4C5)n3)cc3c2n1CC1(CC1)O3]

To a mixture of methyl (4S)-2-[(2S)-3-(4-{2′-[3-(acetyloxy)-2,2-dimethylpropyl]-3′-{2-[(1S)-1-methoxyethyl]-5-[(3R,5S)-3,4,5-trimethylpiperazin-1-yl]pyridin-3-yl}-7′-oxa-4′-azaspiro[cyclopropane-1,6′-tricyclo[6.3.1.0^{4,12}]dodecane]-1′(12′),2′,8′,10′-tetraen-10′-yl}-1,3-thiazol-2-yl)-2-{[(tert-butoxy)carbonyl]amino}propanoyl]-2,3-diazabicyclo[3.1.1]heptane-4-carboxylate (50 mg, 0.051 mmol) in THF (5 mL) was added LiOH (21 mg, 0.51 mmol) and the mixture was stirred at room temperature overnight. The mixture was neutralized with HCl (1 N) until its pH reached 7. The mixture was extracted with EA (10 mL×3). The combined organic layer was washed with brine (10 mL), dried over Na₂SO₄, filtered and concentrated under reduced pressure to give the title compound (45 mg, 95%). LC-MS (M+H)⁺=927.7.

### Step 14: tert-butyl N-[(7′S,13′S)-20′-{2-[(1S)-1-methoxyethyl]-5-[(3R,5S)-3,4,5-trimethylpiperazin-1-yl]pyridin-3-yl}-17′,17′-dimethyl-8′,14′-dioxo-15′,24′-dioxa-4′-thia-9′,21′,30′,32′-tetraazaspiro[cyclopropane-1,23′-heptacyclo[23.3.1.1^{2,5}.1^{9,13}.1^{10,12}.0^{19,27}.0^{21,26}]dotriacontane]-1′(28′),2′,5′(32′),19′,25′(29′),26′-hexaen-7′-yl]carbamate

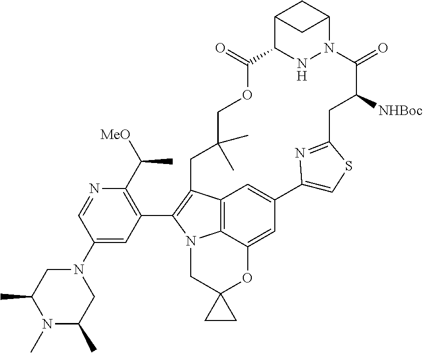 [SMILES: CO[C@@H](C)c1ncc(N2C[C@@H](C)N(C)[C@@H](C)C2)cc1-c1c2c3cc(cc4c3n1CC1(CC1)O4)-c1csc(n1)C[C@H](NC(=O)OC(C)(C)C)C(=O)N1N[C@H](C(=O)OCC(C)(C)C2)C2CC1C2]

A mixture of (4S)-2-[(2S)-2-{[(tert-butoxy)carbonyl]amino}-3-{4-[2′-(3-hydroxy-2,2-dimethylpropyl)-3′-{2-[(1S)-1-methoxyethyl]-5-[(3R,5S)-3,4,5-trimethylpiperazin-1-yl]pyridin-3-yl}-7′-oxa-4′-azaspiro[cyclopropane-1,6′-tricyclo[6.3.1.0^{4,12}]dodecane]-1′(12′),2′,8′,10′-tetraen-10′-yl]-1,3-thiazol-2-yl}propanoyl]-2,3-diazabicyclo[3.1.1]heptane-4-carboxylic acid (45 mg, 0.049 mmol), TCFH (144 mg, 0.51 mmol) and 1-methylimidazole (42 mg, 0.51 mmol) in anhydrous MeCN (20 mL) was stirred at room temperature under nitrogen for 2 h. The mixture was concentrated under reduced pressure. The residue was treated with DCM (5 mL) and water (5 mL). The organic layer was separated, and the aqueous layer was extracted with DCM (5 mL×3). The combined organic layer was washed with brine (5 mL), dried over Na₂SO₄, filtered and concentrated under reduced pressure. The residue was purified by prep-TLC (DCM/MeOH=12/1) to give the title compound (10 mg, 23%). LC-MS (M+H)⁺=909.4.

### Step 15: (1R,2R,3S)—N-[(7′S,13′S)-20′-{2-[(1S)-1-methoxyethyl]-5-[(3R,5S)-3,4,5-trimethylpiperazin-1-yl]pyridin-3-yl}-17′,17′-dimethyl-8′,14′-dioxo-15′,24′-dioxa-4′-thia-9′,21′,30′,32′-tetraazaspiro[cyclopropane-1,23′-heptacyclo[23.3.1.1^{2,5}.1^{9,13}.1^{10,12}.0^{19,27}.0^{21,26}]dotriacontane]-1′(28′),2′,5′(32′),19′,25′(29′),26′-hexaen-7′-yl]-2,3-dimethylcyclopropane-1-carboxamide

To a mixture of tert-butyl N-[(7′S,13′S)-20′-{2-[(1S)-1-methoxyethyl]-5-[(3R,5S)-3,4,5-trimethylpiperazin-1-yl]pyridin-3-yl}-17′,17′-dimethyl-8′,14′-dioxo-15′,24′-dioxa-4′-thia-9′,21′,30′,32′-tetraazaspiro[cyclopropane-1,23′-heptacyclo[23.3.1.1^{2,5}.1^{9,13}.1^{10,12}.0^{19,27}.0^{21,26}]dotriacontane]-1′(28′),2′,5′(32′),19′,25′(29′),26′-hexaen-7′-yl]carbamate (10 mg, 0.011 mmol) in DCM (1 mL) was added HCl (4.0 M in dioxane, 1 mL), and the mixture was stirred at room temperature for 2 h. The mixture was concentrated under reduced pressure. The residue was re-dissolved in DMF (1 mL) followed by addition of (1r,2R,3S)-2,3-dimethylcyclopropane-1-carboxylic acid (2 mg, 0.022 mmol), HATU (8 mg, 0.022 mmol) and DIPEA (4 mg, 0.033 mmol). The mixture was stirred at room temperature for 2 h. The mixture was diluted with water (5 mL) and extracted with EA (5 mL×3). The combined organic layer was dried over Na₂SO₄, filtered and concentrated under reduced pressure. The residue was purified by prep-HPLC to give example 1 (1 mg, 10%). ¹H NMR (400 MHz, DMSO-d6) δ 8.55-8.23 (m, 2H), 8.03 (s, 1H), 7.71 (s, 1H), 7.29-7.14 (m, 1H), 7.09 (s, 1H), 5.90 (d, J=11.1 Hz, 1H), 5.52-5.21 (m, 1H), 4.62 (d, J=11.0 Hz, 1H), 4.49-4.36 (m, 2H), 4.12-3.81 (m, 2H), 3.82-3.44 (m, 4H), 3.19-3.00 (m, 7H), 2.84 (d, J=14.4 Hz, 1H), 2.72-2.54 (m, 2H), 2.37-2.03 (m, 8H), 1.63-1.44 (m, 1H), 1.32 (d, J=6.3 Hz, 3H), 1.23-0.72 (m, 22H), 0.38 (s, 3H). LC-MS (M+H)⁺=905.8.

Atrop-2 & Example 2: (1R,2R,3S)—N-[(7S,13S,19P)-20-{2-[(1S)-1-methoxyethyl]-5-[(3R,5S)-3,4,5-trimethylpiperazin-1-yl]pyridin-3-yl}-17,17-dimethyl-8,14-dioxo-15-oxa-4-thia-9,21,31,33-tetraazaheptacyclo[24.3.1.1^{2,5}.1^{9,13}.1^{10,12}.0^{19,28}.0^{21,27}]tritriaconta-1(29),2,5(33),19,26(30),27-hexaen-7-yl]-2,3-dimethylcyclopropane-1-carboxamide & (1R,2R,3S)—N-[(7S,13S,19M)-20-{2-[(1S)-1-methoxyethyl]-5-[(3R,5S)-3,4,5-trimethylpiperazin-1-yl]pyridin-3-yl}-17,17-dimethyl-8,14-dioxo-15-oxa-4-thia-9,21,31,33-tetraazaheptacyclo[24.3.1.1^{2,5}.1^{9,13}.1^{10,12}.0^{19,28}.0^{21,27}]tritriaconta-1(29),2,5(33),19,26(30),27-hexaen-7-yl]-2,3-dimethylcyclopropane-1-carboxamide

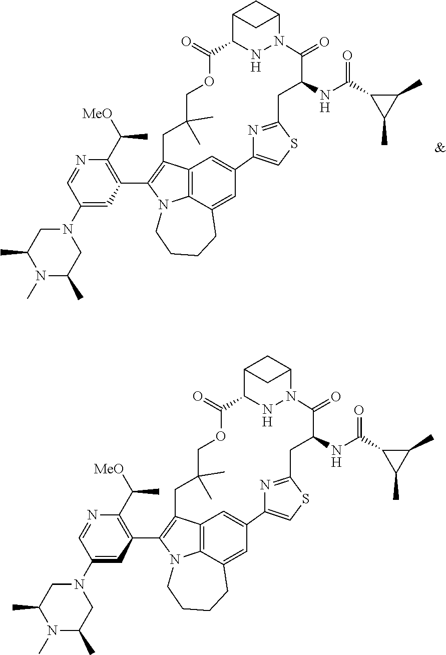

### Step 1: 3-(6-bromo-2-{2-[(1S)-1-methoxyethyl]pyridin-3-yl}-1-azatricyclo[6.4.1.0^{4,13}]trideca-2,4(13),5,7-tetraen-3-yl)-2,2-dimethylpropanoic Acid

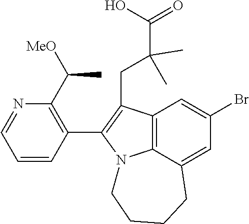 [SMILES: CO[C@@H](C)c1ncccc1-c1c(CC(C)(C)C(=O)O)c2cc(Br)cc3c2n1CCCC3]

The title compound (4.7 g, 96%) was prepared in a manner similar to that in example 1 step 5 from 5-{2-[(1S)-1-methoxyethyl]pyridin-3-yl}-2,2-dimethyl-5-oxopentanoic acid and 7-bromo-2,3,4,5-tetrahydro-1H-benzo[b]azepin-1-amine. LC-MS (M+H)⁺=485.3/487.3.

### Step 2: ethyl 3-(6-bromo-2-{2-[(1S)-1-methoxyethyl]pyridin-3-yl}-1-azatricyclo[6.4.1.0^{4,13}]trideca-2,4(13),5,7-tetraen-3-yl)-2,2-dimethylpropanoate

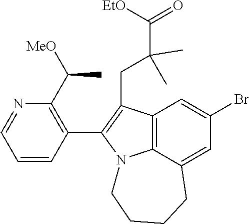 [SMILES: CCOC(=O)C(C)(C)Cc1c(-c2cccnc2[C@H](C)OC)n2c3c(cc(Br)cc13)CCCC2]

The title compound (4.8 g, 96%) was prepared in a manner similar to that in example 1 step 6 from 3-(6-bromo-2-{2-[(1S)-1-methoxyethyl]pyridin-3-yl}-1-azatricyclo[6.4.1.0^{4,13}]trideca-2,4(13),5,7-tetraen-3-yl)-2,2-dimethylpropanoic acid. LC-MS (M+H)⁺=513.3/515.3.

### Step 3: 3-(6-bromo-2-{2-[(1S)-1-methoxyethyl]pyridin-3-yl}-1-azatricyclo[6.4.1.0^{4,13}]trideca-2,4(13),5,7-tetraen-3-yl)-2,2-dimethylpropan-1-ol

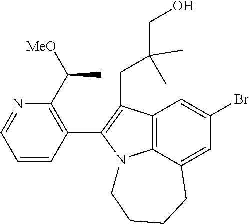 [SMILES: CO[C@@H](C)c1ncccc1-c1c(CC(C)(C)CO)c2cc(Br)cc3c2n1CCCC3]

The title compound (crude form was used in step 4 without further purification) was prepared in a manner similar to that in example 1 step 7 from ethyl 3-(6-bromo-2-{2-[(1S)-1-methoxyethyl]pyridin-3-yl}-1-azatricyclo[6.4.1.0^{4,13}]trideca-2,4(13),5,7-tetraen-3-yl)-2,2-dimethylpropanoate. LC-MS (M+H)⁺=471.3.

### Step 4: 3-(6-bromo-2-{2-[(1S)-1-methoxyethyl]pyridin-3-yl}-1-azatricyclo[6.4.1.0^{4,13}]trideca-2,4(13),5,7-tetraen-3-yl)-2,2-dimethylpropyl Acetate

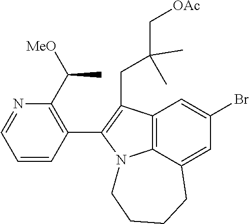 [SMILES: CO[C@@H](C)c1ncccc1-c1c(CC(C)(C)COC(C)=O)c2cc(Br)cc3c2n1CCCC3]

The title compound (4.0 g, 83% over two steps) was prepared in a manner similar to that in example 1 step 8 from 3-(6-bromo-2-{2-[(1S)-1-methoxyethyl]pyridin-3-yl}-1-azatricyclo[6.4.1.0^{4,13}]trideca-2,4(13),5,7-tetraen-3-yl)-2,2-dimethylpropan-1-ol. LC-MS (M+H)⁺=513.4.

### Step 5: (5-{3-[3-(acetyloxy)-2,2-dimethylpropyl]-6-bromo-1-azatricyclo[6.4.1.0^{4,13}]trideca-2,4(13),5,7-tetraen-2-yl}-6-[(1S)-1-methoxyethyl]pyridin-3-yl)boronic Acid

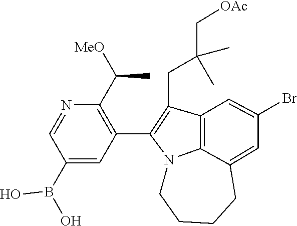 [SMILES: CO[C@@H](C)c1ncc(B(O)O)cc1-c1c(CC(C)(C)COC(C)=O)c2cc(Br)cc3c2n1CCCC3]

The title compound (4.5 g, 89%) was prepared in a manner similar to that in example 1 step 9 from 3-(6-bromo-2-{2-[(1S)-1-methoxyethyl]pyridin-3-yl}-1-azatricyclo[6.4.1.0^{4,13}]trideca-2,4(13),5,7-tetraen-3-yl)-2,2-dimethylpropyl acetate. LC-MS (M+H)⁺=557.4.

### Step 6: 3-(6-bromo-2-{2-[(1S)-1-methoxyethyl]-5-[(3R,5S)-3,4,5-trimethylpiperazin-1-yl]pyridin-3-yl}-1-azatricyclo[6.4.1.0^{4,13}]trideca-2,4(13),5,7-tetraen-3-yl)-2,2-dimethylpropyl Acetate

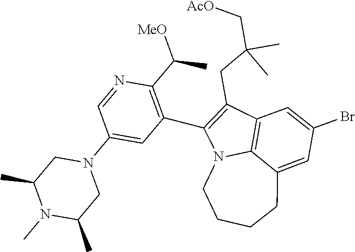 [SMILES: CO[C@@H](C)c1ncc(N2C[C@@H](C)N(C)[C@@H](C)C2)cc1-c1c(CC(C)(C)COC(C)=O)c2cc(Br)cc3c2n1CCCC3]

To a mixture of (5-{3-[3-(acetyloxy)-2,2-dimethylpropyl]-6-bromo-1-azatricyclo[6.4.1.0^{4,13}]trideca-2,4(13),5,7-tetraen-2-yl}-6-[(1S)-1-methoxyethyl]pyridin-3-yl)boronic acid (556 mg, 1.0 mmol) and (2S,6R)-1,2,6-trimethylpiperazine (640 mg, 5.0 mmol) in MeCN (30 mL) was added Et₃N (303 mg, 3.0 mmol) and Cu(OAc)₂ (363 mg, 2.0 mmol). The mixture was stirred at room temperature for 4 h under oxygen and then diluted with EA (50 mL) and water (50 mL). Solid was filtered off, and the filter cake was rinsed with EA (50 mL×2). The organic phase of the filtrate was separated and successively washed with water (50 mL), brine (100 mL), dried over Na₂SO₄, filtered and concentrated under vacuum. The residue was purified by silica gel chromatograph (DCM:MeOH=20:1) to give the title compound (230 mg, 36%). LC-MS (M+H)⁺=639.4.

### Step 7: 3-(2-{2-[(1S)-1-methoxyethyl]-5-[(3R,5S)-3,4,5-trimethylpiperazin-1-yl]pyridin-3-yl}-6-(4,4,5,5-tetramethyl-1,3,2-dioxaborolan-2-yl)-1-azatricyclo[6.4.1.0^{4,13}]trideca-2,4(13),5,7-tetraen-3-yl)-2,2-dimethylpropyl Acetate

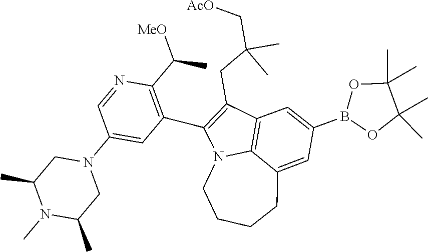

To a mixture of 3-(6-bromo-2-{2-[(1S)-1-methoxyethyl]-5-[(3R,5S)-3,4,5-trimethylpiperazin-1-yl]pyridin-3-yl}-1-azatricyclo[6.4.1.0^{4,13}]trideca-2,4(13),5,7-tetraen-3-yl)-2,2-dimethylpropyl acetate (230 mg, 0.36 mmol) and BPD (113 mg, 0.45 mmol) in dioxane (10 mL) was added Pd(dppf)Cl₂·DCM (27 mg, 0.037 mmol) and KOAc (73 mg, 0.74 mmol). The mixture was stirred at 90° C. for 2 h under nitrogen. The mixture was cooled to room temperature and diluted with EA (40 mL). The mixture was washed with brine (20 mL), dried over Na₂SO₄, filtered and concentrated under vacuum. The residue was purified by silica gel chromatograph (DCM:MeOH=15:1) to give the title compound (100 mg, 39%). LC-MS (M+H)⁺=687.5.

### Step 8: methyl (4S)-2-[(2S)-3-(4-{3-[3-(acetyloxy)-2,2-dimethylpropyl]-2-{2-[(1S)-1-methoxyethyl]-5-[(3R,5S)-3,4,5-trimethylpiperazin-1-yl]pyridin-3-yl}-1-azatricyclo[6.4.1.0^{4,13}]trideca-2,4(13),5,7-tetraen-6-yl}-1,3-thiazol-2-yl)-2-{[(tert-butoxy)carbonyl]amino}propanoyl]-2,3-diazabicyclo[3.1.1]heptane-4-carboxylate

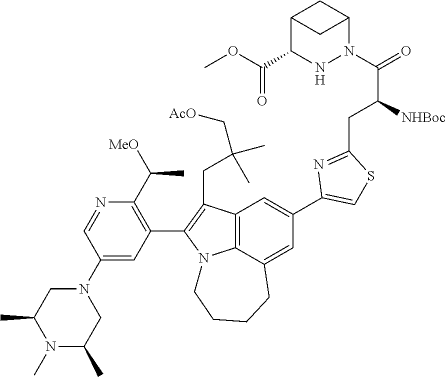 [SMILES: COC(=O)[C@H]1NN(C(=O)[C@H](Cc2nc(-c3cc4c5c(c3)c(CC(C)(C)COC(C)=O)c(-c3cc(N6C[C@@H](C)N(C)[C@@H](C)C6)cnc3[C@H](C)OC)n5CCCC4)cs2)NC(=O)OC(C)(C)C)C2CC1C2]

To a mixture of 3-(2-{2-[(1S)-1-methoxyethyl]-5-[(3R,5S)-3,4,5-trimethylpiperazin-1-yl]pyridin-3-yl}-6-(4,4,5,5-tetramethyl-1,3,2-dioxaborolan-2-yl)-1-azatricyclo[6.4.1.0^{4,13}]trideca-2,4(13),5,7-tetraen-3-yl)-2,2-dimethylpropyl acetate (100 mg, 0.15 mmol) and methyl (4S)-2-[(2S)-3-(4-bromo-1,3-thiazol-2-yl)-2-{[(tert-butoxy)carbonyl]amino}propanoyl]-2,3-diazabicyclo[3.1.1]heptane-4-carboxylate (86 mg, 0.17 mmol) in dioxane (5 mL) and water (0.5 mL) was added K₃PO₄ (64 mg, 0.3 mmol) and Pd(dtbpf)Cl₂ (10 mg, 0.015 mmol). The mixture was stirred for 3 h at 70° C. under nitrogen and cooled to room temperature. The mixture was diluted with EA (40 mL), washed with water (20 mL), brine (20 mL), dried over Na₂SO₄, filtered and concentrated under vacuum. The residue was purified by silica gel chromatograph (DCM:MeOH=10:1) to give the title compound (100 mg, 69%). LC-MS (M+H)⁺=969.5.

### Step 9: (4S)-2-[(2S)-2-{[(tert-butoxy)carbonyl]amino}-3-{4-[3-(3-hydroxy-2,2-dimethylpropyl)-2-{2-[(1S)-1-methoxyethyl]-5-[(3R,5S)-3,4,5-trimethylpiperazin-1-yl]pyridin-3-yl}-1-azatricyclo[6.4.1.0^{4,13}]trideca-2,4(13),5,7-tetraen-6-yl]-1,3-thiazol-2-yl}propanoyl]-2,3-diazabicyclo[3.1.1]heptane-4-carboxylic Acid

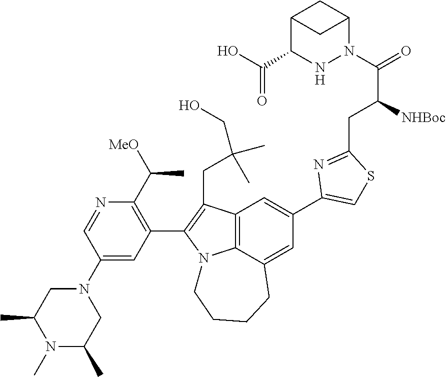 [SMILES: CO[C@@H](C)c1ncc(N2C[C@@H](C)N(C)[C@@H](C)C2)cc1-c1c(CC(C)(C)CO)c2cc(-c3csc(C[C@H](NC(=O)OC(C)(C)C)C(=O)N4N[C@H](C(=O)O)C5CC4C5)n3)cc3c2n1CCCC3]

To a mixture of methyl (4S)-2-[(2S)-3-(4-{3-[3-(acetyloxy)-2,2-dimethylpropyl]-2-{2-[(1S)-1-methoxyethyl]-5-[(3R,5S)-3,4,5-trimethylpiperazin-1-yl]pyridin-3-yl}-1-azatricyclo[6.4.1.0^{4,13}]trideca-2,4(13),5,7-tetraen-6-yl}-1,3-thiazol-2-yl)-2-{[(tert-butoxy)carbonyl]amino}propanoyl]-2,3-diazabicyclo[3.1.1]heptane-4-carboxylate (100 mg, 0.10 mmol) in THF (10 mL) and water (10 mL) was added LiOH (13 mg, 0.30 mmol). The mixture was stirred at room temperature for 3 h. The mixture was then diluted with water (30 mL) and washed with EA (10 mL). The organic layer was discarded and the pH of aqueous layer was adjusted to ˜5 with 1 N hydrochloric acid at 0° C. The mixture was extracted with EA (30 mL×3). The combined organic layer was dried over Na₂SO₄, filtered and concentrated under vacuum to give the title compound (46 mg, 50%). LC-MS (M+H)⁺=913.5.

### Step 10: tert-butyl N-[(7S,13S)-20-{2-[(1S)-1-methoxyethyl]-5-[(3R,5S)-3,4,5-trimethylpiperazin-1-yl]pyridin-3-yl}-17,17-dimethyl-8,14-dioxo-15-oxa-4-thia-9,21,31,33-tetraazaheptacyclo[24.3.1.1^{2,5}.1^{9,13}.1^{10,12}.0^{19,28}.0^{21,27}]tritriaconta-1(29),2,5(33),19,26(30),27-hexaen-7-yl]carbamate

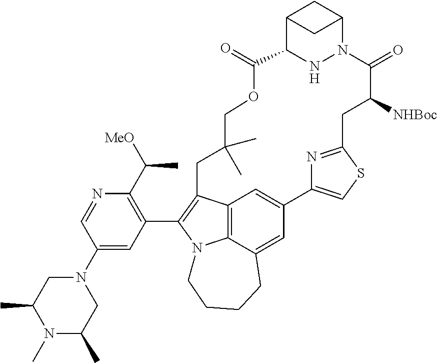 [SMILES: CO[C@@H](C)c1ncc(N2C[C@@H](C)N(C)[C@@H](C)C2)cc1-c1c2c3cc(cc4c3n1CCCC4)-c1csc(n1)C[C@H](NC(=O)OC(C)(C)C)C(=O)N1N[C@H](C(=O)OCC(C)(C)C2)C2CC1C2]

To a mixture of (4S)-2-[(2S)-2-{[(tert-butoxy)carbonyl]amino}-3-{4-[3-(3-hydroxy-2,2-dimethylpropyl)-2-{2-[(1S)-1-methoxyethyl]-5-[(3R,5S)-3,4,5-trimethylpiperazin-1-yl]pyridin-3-yl}-1-azatricyclo[6.4.1.0^{4,13}]trideca-2,4(13),5,7-tetraen-6-yl]-1,3-thiazol-2-yl}propanoyl]-2,3-diazabicyclo[3.1.1]heptane-4-carboxylic acid (46 mg, 0.05 mmol) in MeCN (20 mL) was added TCFH (36 mg, 0.12 mmol) and NMI (21 mg, 0.25 mmol). The mixture was stirred at room temperature for 20 h and then washed with water (10 mL) and brine (10 mL). The combined organic layer was dried over Na₂SO₄, filtered and concentrated under vacuum. The residue was purified by silica gel chromatograph (DCM:MeOH=20:1) to give the title compound (30 mg, 67%). LC-MS (M+H)⁺=895.5.

### Step 11: (7S,13S)-7-amino-20-{2-[(1S)-1-methoxyethyl]-5-[(3R,5S)-3,4,5-trimethylpiperazin-1-yl]pyridin-3-yl}-17,17-dimethyl-15-oxa-4-thia-9,21,31,33-tetraazaheptacyclo[24.3.1.1^{2,5}.1^{9,13}.1^{10,12}.0^{19,28}.0^{21,27}]tritriaconta-1(29),2,5(33),19,26(30),27-hexaene-8,14-dione

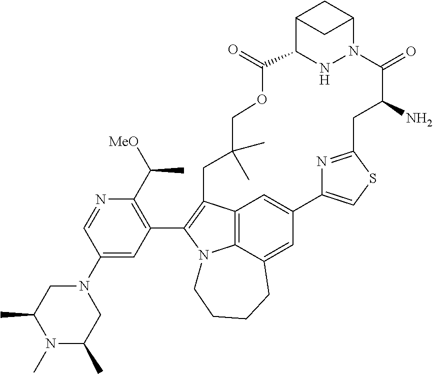 [SMILES: CO[C@@H](C)c1ncc(N2C[C@@H](C)N(C)[C@@H](C)C2)cc1-c1c2c3cc(cc4c3n1CCCC4)-c1csc(n1)C[C@H](N)C(=O)N1N[C@H](C(=O)OCC(C)(C)C2)C2CC1C2]

A mixture of tert-butyl N-[(7S,13S)-20-{2-[(1S)-1-methoxyethyl]-5-[(3R,5S)-3,4,5-trimethylpiperazin-1-yl]pyridin-3-yl}-17,17-dimethyl-8,14-dioxo-15-oxa-4-thia-9,21,31,33-tetraazaheptacyclo[24.3.1.1^{2,5}.1^{9,13}.1^{10,12}.0^{19,28}.0^{21,27}]tritriaconta-1(29),2,5(33),19,26(30),27-hexaen-7-yl]carbamate (30 mg, 0.034 mmol) in HCl (4 M in dioxane, 3 mL) was stirred at room temperature for 1 h and then concentrated under vacuum. The residue was partitioned between DCM (10 mL) and saturated NaHCO₃ (5 mL). The organic layer was dried over Na₂SO₄, filtered, and concentrated under vacuum to give the title compound (20 mg, 74%). LC-MS (M+H)⁺=795.5.

### Step 12: (1R,2R,3S)—N-[(7S,13S,19P)-20-{2-[(1S)-1-methoxyethyl]-5-[(3R,5S)-3,4,5-trimethylpiperazin-1-yl]pyridin-3-yl}-17,17-dimethyl-8,14-dioxo-15-oxa-4-thia-9,21,31,33-tetraazaheptacyclo[24.3.1.1^{2,5}.1^{9,13}.1^{10,12}.0^{19,28}.0^{21,27}]tritriaconta-1(29),2,5(33),19,26(30),27-hexaen-7-yl]-2,3-dimethylcyclopropane-1-carboxamide & (1R,2R,3S)—N-[(7S,13S,19M)-20-{2-[(1S)-1-methoxyethyl]-5-[(3R,5S)-3,4,5-trimethylpiperazin-1-yl]pyridin-3-yl}-17,17-dimethyl-8,14-dioxo-15-oxa-4-thia-9,21,31,33-tetraazaheptacyclo[24.3.1.1^{2,5}.1^{9,13}.1^{10,12}.0^{19,28}.0^{21,27}]tritriaconta-1(29),2,5(33),19,26(30),27-hexaen-7-yl]-2,3-dimethylcyclopropane-1-carboxamide

To a mixture of (7S,13S)-7-amino-20-{2-[(1S)-1-methoxyethyl]-5-[(3R,5S)-3,4,5-trimethylpiperazin-1-yl]pyridin-3-yl}-17,17-dimethyl-15-oxa-4-thia-9,21,31,33-tetraazaheptacyclo[24.3.1.1^{2,5}.1^{9,13}.1^{10,12}.0^{19,28}.0^{21,27}]tritriaconta-1(29),2,5(33),19,26(30),27-hexaene-8,14-dione (20 mg, 0.025 mmol) in DMF (3 mL) was added DIPEA (13 mg, 0.10 mmol), (1r,2R,3S)-2,3-dimethylcyclopropane-1-carboxylic acid (5 mg, 0.040 mmol) and HATU (14 mg, 0.038 mmol). The mixture was stirred for 1 h at room temperature and diluted with EA (15 mL). The mixture was washed with brine (10 mL), dried over Na₂SO₄, filtered, and concentrated under vacuum. The residue was purified by prep-TLC (DCM:MeOH=10:1) followed by prep-HPLC to give atrop-2 (P-isomer, 0.4 mg, 2%) and example 2 (M-isomer, 1.7 mg, 8%).

Atrop-2: ¹H NMR (400 MHz, DMSO, d6) δ 8.46-8.40 (m, 2H), 8.23 (s, 1H), 7.76 (s, 1H), 7.46 (s, 1H), 7.32 (s, 1H), 5.90-5.87 (m, 1H), 5.32-5.30 (m, 2H), 4.72-4.69 (m, 1H), 4.48 (s, 1H), 3.91-3.89 (m, 2H), 3.76-3.48 (m, 4H), 3.22-3.15 (m, 2H), 3.07 (s, 3H), 2.99-2.97 (m, 2H), 2.70-2.60 (m, 3H), 2.40-2.33 (m, 5H), 2.21-2.17 (m, 6H), 2.08-1.93 (m, 4H), 1.91-1.82 (m, 2H), 1.61 (s, 2H), 1.45 (s, 2H), 1.19-1.13 (m, 6H), 1.12-0.99 (m, 6H), 0.90 (s, 3H), 0.52 (s, 3H). LC-MS (M+H)⁺=891.3.

Example 2: ¹H NMR (400 MHz, DMSO, d6) δ 8.38 (d, J=2.6 Hz, 2H), 8.19 (s, 1H), 7.73 (s, 1H), 7.46 (s, 1H), 7.13 (s, 1H), 5.90-5.88 (m, 1H), 5.32 (s, 1H), 4.60-4.57 (m, 1H), 4.44-4.42 (m, 1H), 4.29-4.25 (m, 1H), 4.06-4.04 (m, 1H), 3.69-3.66 (m, 3H), 3.49-3.47 (m, 3H), 3.18 (s, 3H), 3.13-2.99 (m, 4H), 2.86-2.84 (m, 2H), 2.63-2.61 (m, 2H), 2.58-2.56 (m, 1H), 2.28 (s, 3H), 2.24-2.05 (m, 4H), 1.96-1.93 (m, 4H), 1.68-1.65 (m, 2H), 1.52-1.49 (m, 2H), 1.30-1.29 (m, 4H), 1.14-1.10 (m, 4H), 1.05-1.00 (m, 7H), 0.83-0.80 (m, 3H), 0.29 (s, 3H). LC-MS (M+H)⁺=891.3.

### Example 3 & Example 4: (1S,2S)—N-[(7′S,13′S)-20′-{5-[(2R,6S)-2,6-dimethylmorpholin-4-yl]-2-[(1S)-1-methoxyethyl]pyridin-3-yl}-17′,17′-dimethyl-8′,14′-dioxo-15′,24′-dioxa-4′-thia-9′,21′,30′,32′-tetraazaspiro[cyclopropane-1,23′-heptacyclo[23.3.1.1^{2,5}.1^{9,13}.1^{10,12}.0^{19,27}.0^{21,26}]dotriacontane]-1′(28′),2′,5′(32′),19′,25′(29′),26′-hexaen-7′-yl]-2-methylcyclopropane-1-carboxamide & (1R,2R,3S)—N-[(7′S,13′S)-20′-{5-[(2R,6S)-2,6-dimethylmorpholin-4-yl]-2-[(1S)-1-methoxyethyl]pyridin-3-yl}-17′,17′-dimethyl-8′,14′-dioxo-15′,24′-dioxa-4′-thia-9′,21′,30′,32′-tetraazaspiro[cyclopropane-1,23′-heptacyclo[23.3.1.1^{2,5}.1^{9,13}.1^{10,12}.0^{19,27}.0^{21,26}]dotriacontane]-1′(28′),2′,5′(32′),19′,25′(29′),26′-hexaen-7′-yl]-2,3-dimethylcyclopropane-1-carboxamide

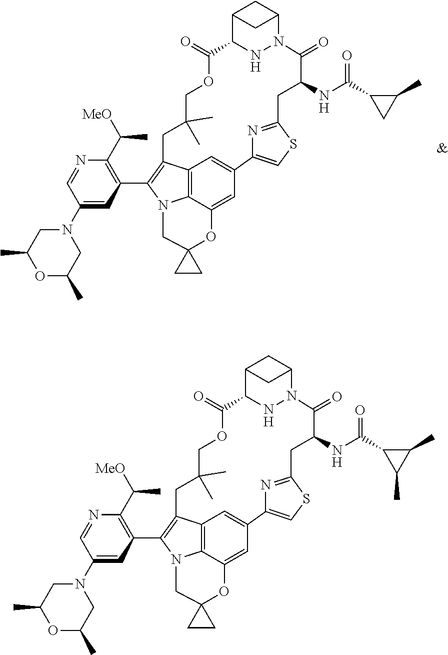

### Step 1: 3-(10′-bromo-3′-{5-[(2R,6S)-2,6-dimethylmorpholin-4-yl]-2-[(1S)-1-methoxyethyl]pyridin-3-yl}-7′-oxa-4′-azaspiro[cyclopropane-1,6′-tricyclo[6.3.1.0^{4,12}]dodecane]-1′(12′),2′,8′,10′-tetraen-2′-yl)-2,2-dimethylpropyl Acetate

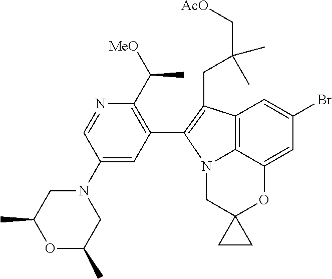 [SMILES: CO[C@@H](C)c1ncc(N2C[C@@H](C)O[C@@H](C)C2)cc1-c1c(CC(C)(C)COC(C)=O)c2cc(Br)cc3c2n1CC1(CC1)O3]

The title compound (550 mg, 70%) was prepared in a manner similar to that in example 1 step 10 from 5-{2′-[3-(acetyloxy)-2,2-dimethylpropyl]-10′-bromo-7′-oxa-4′-azaspiro[cyclopropane-1,6′-tricyclo[6.3.1.0^{4,12}]dodecane]-1′(12′),2′,8′,10′-tetraen-3′-yl}-6-[(1S)-1-methoxyethyl]pyridin-3-yl)boronic acid and (2R,6S)-2,6-dimethylmorpholine. LC-MS (M+H)⁺=640.4.

### Step 2: 3-(3′-{5-[(2R,6S)-2,6-dimethylmorpholin-4-yl]-2-[(1S)-1-methoxyethyl]pyridin-3-yl}-10′-(4,4,5,5-tetramethyl-1,3,2-dioxaborolan-2-yl)-7′-oxa-4′-azaspiro[cyclopropane-1,6′-tricyclo[6.3.1.0^{4,12}]dodecane]-1′(12′),2′,8′,10′-tetraen-2′-yl)-2,2-dimethylpropyl Acetate

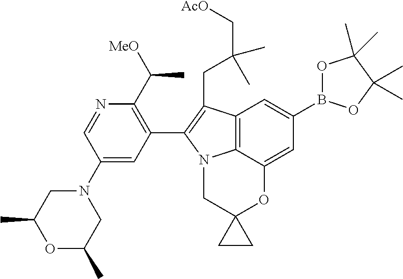

The title compound (crude form was used in step 3 without further purification) was prepared in a manner similar to that in example 1 step 11 from 3-(10′-bromo-3′-{5-[(2R,6S)-2,6-dimethylmorpholin-4-yl]-2-[(1S)-1-methoxyethyl]pyridin-3-yl}-7′-oxa-4′-azaspiro[cyclopropane-1,6′-tricyclo[6.3.1.0^{4,12}]dodecane]-1′(12′),2′,8′,10′-tetraen-2′-yl)-2,2-dimethylpropyl acetate. LC-MS (M+H)⁺=688.5.

### Step 3: methyl (4S)-2-[(2S)-3-(4-{2′-[3-(acetyloxy)-2,2-dimethylpropyl]-3′-{5-[(2R,6S)-2,6-dimethylmorpholin-4-yl]-2-[(1S)-1-methoxyethyl]pyridin-3-yl}-7′-oxa-4′-azaspiro[cyclopropane-1,6′-tricyclo[6.3.1.0^{4,12}]dodecane]-1′(12′),2′,8′,10′-tetraen-10′-yl}-1,3-thiazol-2-yl)-2-{[(tert-butoxy)carbonyl]amino}propanoyl]-2,3-diazabicyclo[3.1.1]heptane-4-carboxylate

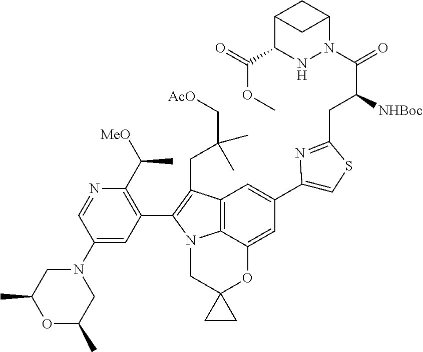 [SMILES: COC(=O)[C@H]1NN(C(=O)[C@H](Cc2nc(-c3cc4c5c(c3)c(CC(C)(C)COC(C)=O)c(-c3cc(N6C[C@@H](C)O[C@@H](C)C6)cnc3[C@H](C)OC)n5CC3(CC3)O4)cs2)NC(=O)OC(C)(C)C)C2CC1C2]

The title compound (120 mg, 21% over 2 steps) was prepared in a manner similar to that in example 1 step 12 from 3-(3′-{5-[(2R,6S)-2,6-dimethylmorpholin-4-yl]-2-[(1S)-1-methoxyethyl]pyridin-3-yl}-10′-(4,4,5,5-tetramethyl-1,3,2-dioxaborolan-2-yl)-7′-oxa-4′-azaspiro[cyclopropane-1,6′-tricyclo[6.3.1.0^{4,12}]dodecane]-1′(12′),2′,8′,10′-tetraen-2′-yl)-2,2-dimethylpropyl acetate (crude directly from step 2) and methyl (4S)-2-[(2S)-3-(4-bromo-1,3-thiazol-2-yl)-2-{[(tert-butoxy)carbonyl]amino}propanoyl]-2,3-diazabicyclo[3.1.1]heptane-4-carboxylate. LC-MS (M+H)⁺=970.6.

### Step 4: (4S)-2-[(2S)-2-{[(tert-butoxy)carbonyl]amino}-3-[4-(3′-{5-[(2R,6S)-2,6-dimethylmorpholin-4-yl]-2-[(1S)-1-methoxyethyl]pyridin-3-yl}-2′-(3-hydroxy-2,2-dimethylpropyl)-7′-oxa-4′-azaspiro[cyclopropane-1,6′-tricyclo[6.3.1.0^{4,12}]dodecane]-1′(12′),2′,8′,10′-tetraen-10′-yl)-1,3-thiazol-2-yl]propanoyl]-2,3-diazabicyclo[3.1.1]heptane-4-carboxylic Acid

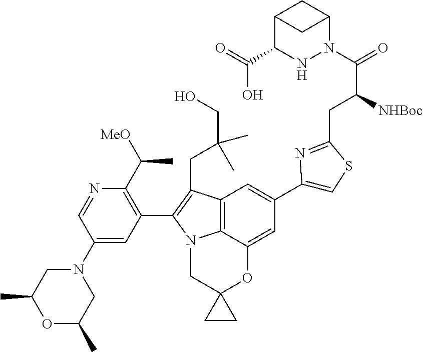 [SMILES: CO[C@@H](C)c1ncc(N2C[C@@H](C)O[C@@H](C)C2)cc1-c1c(CC(C)(C)CO)c2cc(-c3csc(C[C@H](NC(=O)OC(C)(C)C)C(=O)N4N[C@H](C(=O)O)C5CC4C5)n3)cc3c2n1CC1(CC1)O3]

The title compound (100 mg, 88%) was prepared in a manner similar to that in example 1 step 13 from methyl (4S)-2-[(2S)-3-(4-{2′-[3-(acetyloxy)-2,2-dimethylpropyl]-3′-{5-[(2R,6S)-2,6-dimethylmorpholin-4-yl]-2-[(1S)-1-methoxyethyl]pyridin-3-yl}-7′-oxa-4′-azaspiro[cyclopropane-1,6′-tricyclo[6.3.1.04,]dodecane]-1′(12′),2′,8′,10′-tetraen-10′-yl}-1,3-thiazol-2-yl)-2-{[(tert-butoxy)carbonyl]amino}propanoyl]-2,3-diazabicyclo[3.1.1]heptane-4-carboxylate. LC-MS (M+H)⁺=914.7.

### Step 5: tert-butyl N-[(7′S,13′S)-20′-{5-[(2R,6S)-2,6-dimethylmorpholin-4-yl]-2-[(1S)-1-methoxyethyl]pyridin-3-yl}-17′,17′-dimethyl-8′,14′-dioxo-15′,24′-dioxa-4′-thia-9′,21′,30′,32′-tetraazaspiro[cyclopropane-1,23′-heptacyclo[23.3.1.1^{2,5}.1^{9,13}.1^{10,12}.0^{19,27}.0^{21,26}]dotriacontane]-1′(28′),2′,5′(32′),19′,25′(29′),26′-hexaen-7′-yl]carbamate

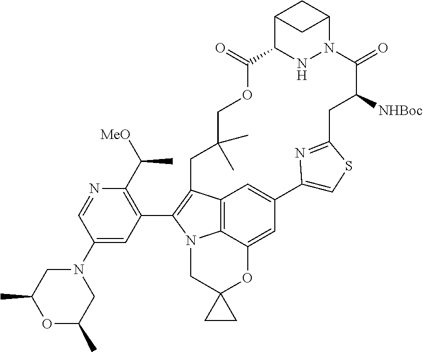 [SMILES: CO[C@@H](C)c1ncc(N2C[C@@H](C)O[C@@H](C)C2)cc1-c1c2c3cc(cc4c3n1CC1(CC1)O4)-c1csc(n1)C[C@H](NC(=O)OC(C)(C)C)C(=O)N1N[C@H](C(=O)OCC(C)(C)C2)C2CC1C2]

The title compound (30 mg, 31%) was prepared in a manner similar to that in example 1 step 14 from (4S)-2-[(2S)-2-{[(tert-butoxy)carbonyl]amino}-3-[4-(3′-{5-[(2R,6S)-2,6-dimethylmorpholin-4-yl]-2-[(1S)-1-methoxyethyl]pyridin-3-yl}-2′-(3-hydroxy-2,2-dimethylpropyl)-7′-oxa-4′-azaspiro[cyclopropane-1,6′-tricyclo[6.3.1.0^{4,12}]dodecane]-1′(12′),2′,8′,10′-tetraen-10′-yl)-1,3-thiazol-2-yl]propanoyl]-2,3-diazabicyclo[3.1.1]heptane-4-carboxylic acid. LC-MS (M+H)⁺=896.7.

### Step 6: (1S,2S)—N-[(7′S,13′S)-20′-{5-[(2R,6S)-2,6-dimethylmorpholin-4-yl]-2-[(1S)-1-methoxyethyl]pyridin-3-yl}-17′,17′-dimethyl-8′,14′-dioxo-15′,24′-dioxa-4′-thia-9′,21′,30′,32′-tetraazaspiro[cyclopropane-1,23′-heptacyclo[23.3.1.1^{2,5}.1^{9,13}.1^{10,12}.0^{19,27}.0^{21,26}]dotriacontane]-1′(28′),2′,5′(32′),19′,25′(29′),26′-hexaen-7′-yl]-2-methylcyclopropane-1-carboxamide & (1R,2R,3S)—N-[(7′S,13′S)-20′-{5-[(2R,6S)-2,6-dimethylmorpholin-4-yl]-2-[(1S)-1-methoxyethyl]pyridin-3-yl}-17′,17′-dimethyl-8′,14′-dioxo-15′,24′-dioxa-4′-thia-9′21′30′,32′-tetraazaspiro[cyclopropane-1,23′-heptacyclo[23.3.1.1^{2,5}.1^{9,13}.1^{10,12}.0^{19,27}.0^{21,26}]dotriacontane]-1′(28′),2′,5′(32′),19′,25′(29′),26′-hexaen-7′-yl]-2,3-dimethylcyclopropane-1-carboxamide

To a mixture of tert-butyl N-[(7′S,13′S)-20′-{5-[(2R,6S)-2,6-dimethylmorpholin-4-yl]-2-[(1S)-1-methoxyethyl]pyridin-3-yl}-17′,17′-dimethyl-8′,14′-dioxo-15′,24′-dioxa-4′-thia-9′,21′,30′,32′-tetraazaspiro[cyclopropane-1,23′-heptacyclo[23.3.1.1^{2,5}.1^{9,13}.1^{10,12}.0^{19,27}.0^{21,26}]dotriacontane]-1′(28′),2′,5′(32′),19′,25′(29′),26′-hexaen-7′-yl]carbamate (30 mg, 0.034 mmol) in DCM (1 mL) was added HCl (4.0 M in dioxane, 1 mL), and the mixture was stirred at room temperature for 2 h. The mixture was concentrated under reduced pressure to give a residue (25 mg).

A portion of the residue (10 mg) was re-dissolved in DMF (1 mL) followed by addition of (1S,2S)-2-methylcyclopropane-1-carboxylic acid (2 mg, 0.020 mmol), HATU (8 mg, 0.020 mmol) and DIPEA (4 mg, 0.030 mmol). The mixture was stirred at room temperature for 2 h. The mixture was diluted with water (5 mL) and extracted with EA (5 mL×3). The combined organic layer was dried over Na₂SO₄, filtered and concentrated under reduced pressure. The residue was purified by prep-HPLC to give example 3 (4 mg, 34%). ¹H NMR (400 MHz, DMSO-d6) δ 8.48 (d, J=8.9 Hz, 1H), 8.39 (d, J=2.9 Hz, 1H), 8.03 (s, 1H), 7.71 (s, 1H), 7.18 (d, J=2.9 Hz, 1H), 7.09 (s, 1H), 5.91 (d, J=11.0 Hz, 1H), 5.35 (t, J=8.4 Hz, 1H), 4.63 (d, J=11.0 Hz, 1H), 4.52-4.33 (m, 2H), 4.01-3.77 (m, 2H), 3.81-3.58 (m, 5H), 3.51 (d, J=10.7 Hz, 1H), 3.25-2.96 (m, 5H), 2.85 (d, J=14.3 Hz, 1H), 2.67-2.57 (m, 2H), 2.43-2.39 (m, 1H), 2.35-2.20 (m, 3H), 2.10 (t, J=9.8 Hz, 1H), 1.51 (t, J=9.3 Hz, 1H), 1.47-1.41 (m, 1H), 1.33 (d, J=6.3 Hz, 3H), 1.21-1.17 (m, 1H), 1.13-1.07 (m, 5H), 1.02 (s, 6H), 0.86 (s, 6H), 0.53-0.45 (m, 1H), 0.39 (s, 3H). LC-MS (M+H)⁺=878.7.

The other portion of the Boc-deprotected residue (15 mg) was re-dissolved in DMF (1 mL) followed by addition of (1r,2R,3S)-2,3-dimethylcyclopropane-1-carboxylic acid (4 mg, 0.040 mmol), HATU (15 mg, 0.040 mmol) and DIPEA (8 mg, 0.060 mmol). The mixture was stirred at room temperature for 2 h. The mixture was diluted with water (5 mL) and extracted with EA (5 mL×3). The combined organic layer was dried over Na₂SO₄, filtered and concentrated under reduced pressure. The residue was purified by prep-HPLC to give example 4 (3 mg, 17%).

¹H NMR (400 MHz, DMSO-d6) δ 8.52-8.30 (m, 2H), 8.03 (s, 1H), 7.84-7.66 (m, 1H), 7.18 (s, 1H), 7.09 (s, 1H), 5.90 (d, J=10.9 Hz, 1H), 5.42-5.28 (m, 1H), 4.86-4.54 (m, 1H), 4.46-4.32 (m, 2H), 4.05-3.78 (m, 2H), 3.76-3.57 (m, 5H), 3.51 (d, J=10.7 Hz, 1H), 3.23-3.01 (m, 5H), 2.84 (d, J=14.5 Hz, 1H), 2.71-2.54 (m, 2H), 2.51-2.48 (m, 1H), 2.37-2.20 (m, 3H), 2.10 (t, J=9.7 Hz, 1H), 1.55-1.43 (m, 1H), 1.32 (d, J=6.3 Hz, 3H), 1.20-0.96 (m, 16H), 0.85 (s, 6H), 0.39 (s, 3H). LC-MS (M+H)⁺=892.7.

Atrop-5 & Example 5: (1R,2R,3S)—N-[(7S,13S,19P)-20-{2-[(1S)-1-methoxyethyl]-5-(2,2,6,6-tetramethyloxan-4-yl)pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide & (1R,2R,3S)—N-[(7S,13S,19M)-20-{2-[(1S)-1-methoxyethyl]-5-(2,2,6,6-tetramethyloxan-4-yl)pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

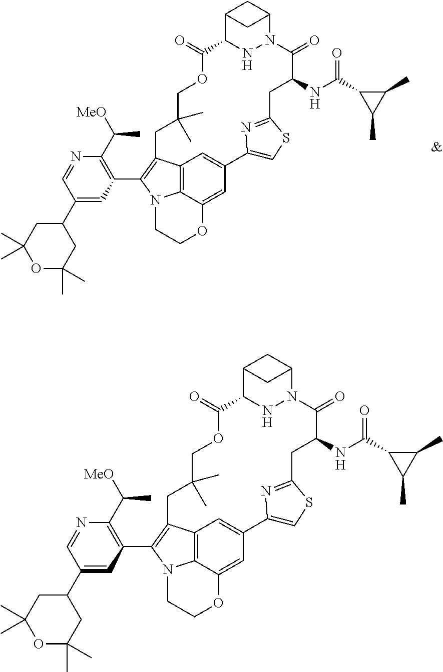

### Step 1: 4-methyl-N′-(2,2,6,6-tetramethyloxan-4-ylidene)benzene-1-sulfonohydrazide

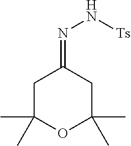

A mixture of 2,2,6,6-tetramethyloxan-4-one (3.0 g, 19.2 mmol) and tosylhydrazide (3.58 g, 19.2 mmol) in anhydrous MeOH (50 mL) was stirred at 60° C. for 2 h. The mixture was cooled to room temperature and concentrated under reduced pressure to give the title compound (6.1 g, 98%). LC-MS (M+H)⁺=325.3.

### Step 2: 7-bromo-3,4-dihydro-2H-1,4-benzoxazin-4-amine

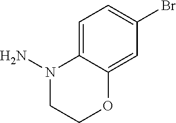 [SMILES: NN1CCOc2cc(Br)ccc21]

To a solution of 7-bromo-3,4-dihydro-2H-1,4-benzoxazine (24.0 g, 112 mmol) in EtOH (120 mL) was added a solution of NaNO₂ (9.33 g, 135 mmol) in water (50 mL). The mixture was cooled to 0° C., and concentrated hydrochloric acid (12.2 mL) was dropwise added at 0° C. with vigorously stirring. The mixture was stirred for 30 min at 0° C. A solution of NaOH (45.1 g, 1.12 mol) in water (120 mL) was added to the mixture at 0° C. followed by solid Na₂S₂O₄ (85%, 69.2 g, 338.1 mmol). The mixture was heated to reflux for 2 h and then cooled to room temperature, diluted with water (500 mL) and extracted with EA (700 mL×2). The combined organic layer was washed with brine (100 mL), dried over Na₂SO₄, filtered and concentrated under vacuum. The residue was purified by silica gel chromatograph (PE:EA=1:2) to give the title compound (11.0 g, 42%). LC-MS (M+H)⁺=229.1.

### Step 3: 3-(6-bromo-2-{2-[(1S)-1-methoxyethyl]pyridin-3-yl}-9-oxa-1-azatricyclo[6.3.1.0^{4,12}]dodeca-2,4(12),5,7-tetraen-3-yl)-2,2-dimethylpropanoic Acid

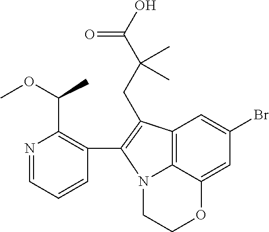 [SMILES: CO[C@@H](C)c1ncccc1-c1c(CC(C)(C)C(=O)O)c2cc(Br)cc3c2n1CCO3]

To a mixture of 7-bromo-3,4-dihydro-2H-1,4-benzoxazin-4-amine (9.0 g, 39.3 mmol) and 5-{2-[(1S)-1-methoxyethyl]pyridin-3-yl}-2,2-dimethyl-5-oxopentanoic acid (11.0 g, 39.4 mmol) in toluene (500 mL) was added TsOH·H₂O (15.0 g, 78.9 mmol) at room temperature. The mixture was stirred for 16 h at 100° C. under nitrogen then cooled to room temperature. The mixture was concentrated under reduced pressure. The residue was diluted with EA (400 mL) and successively washed with water (150 mL) and brine (100 mL). The organic layer was dried over Na₂SO₄, filtered and concentrated under vacuum. The residue was purified by silica gel chromatograph (MeOH:EA=1:100) to give the title compound (6.6 g, 36%). LC-MS (M+H)⁺=473.1.

### Step 4: ethyl 3-(6-bromo-2-{2-[(1S)-1-methoxyethyl]pyridin-3-yl}-9-oxa-1-azatricyclo[6.3.1.0^{4,12}]dodeca-2,4(12),5,7-tetraen-3-yl)-2,2-dimethylpropanoate

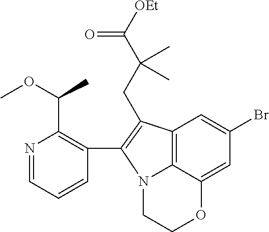 [SMILES: CCOC(=O)C(C)(C)Cc1c(-c2cccnc2[C@H](C)OC)n2c3c(cc(Br)cc13)OCC2]

To a solution of 3-(6-bromo-2-(2-[(1S)-1-methoxyethyl]pyridin-3-yl)-9-oxa-1-azatricyclo[6.3.1.0^{4,12}]dodeca-2,4(12),5,7-tetraen-3-yl)-2,2-dimethylpropanoic acid (6.57 g, 13.9 mmol) in DMF (100 mL) was added Cs₂CO₃ (6.79 g, 20.8 mmol). EtI (3.24 g, 20.8 mmol) was added dropwise at 0° C. under nitrogen. The mixture was stirred for 2 h at room temperature and then diluted with EA (300 mL). The mixture was washed with brine (3×80 mL), dried over Na₂SO₄, filtered and concentrated under vacuum. The residue was purified by silica gel chromatography (PE:EA=1:4) to give the title compound (6.8 g, 98%). LC-MS (M+H)⁺=501.1.

### Step 5: 3-(6-bromo-2-{2-[(1S)-1-methoxyethyl]pyridin-3-yl}-9-oxa-1-azatricyclo[6.3.1.0^{4,12}]dodeca-2,4(12),5,7-tetraen-3-yl)-2,2-dimethylpropan-1-ol

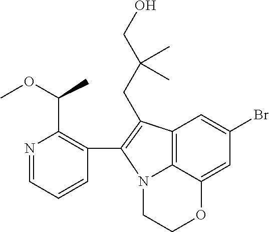 [SMILES: CO[C@@H](C)c1ncccc1-c1c(CC(C)(C)CO)c2cc(Br)cc3c2n1CCO3]

To a solution of ethyl 3-(6-bromo-2-(2-[(1S)-1-methoxyethyl]pyridin-3-yl)-9-oxa-1-azatricyclo[6.3.1.0^{4,12}]dodeca-2,4(12),5,7-tetraen-3-yl)-2,2-dimethylpropanoate (6.8 g, 13.6 mmol) in THF (100 mL) at 0° C. was added LiBH₄ (1.48 g, 67.9 mmol) under nitrogen. The mixture was stirred at 60° C. for 16 h. The mixture was cooled to room temperature and carefully quenched with pre-cooled (0° C.) saturated NH₄Cl (80 mL). The mixture was extracted with EA (150 mL×2) and the combined organic layer was washed with brine (60 mL×2), dried over Na₂SO₄, filtered and concentrated under vacuum. The residue was purified by silica gel chromatograph (EA:MeOH=200:1) to give the title compound (5.3 g, 85%). LC-MS (M+H)⁺=459.1.

### Step 6: 3-(6-bromo-2-{2-[(1S)-1-methoxyethyl]pyridin-3-yl}-9-oxa-1-azatricyclo[6.3.1.0^{4,12}]dodeca-2,4(12),5,7-tetraen-3-yl)-2,2-dimethylpropyl Acetate

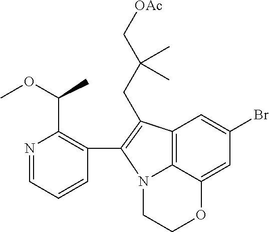 [SMILES: CO[C@@H](C)c1ncccc1-c1c(CC(C)(C)COC(C)=O)c2cc(Br)cc3c2n1CCO3]

To a mixture of 3-(6-bromo-2-{2-[(1S)-1-methoxyethyl]pyridin-3-yl}-9-oxa-1-azatricyclo[6.3.1.0^{4,12}]dodeca-2,4(12),5,7-tetraen-3-yl)-2,2-dimethylpropan-1-ol (5.3 g, 11.5 mmol), Et₃N (2.33 g, 23.1 mmol) and DMAP (140 mg, 1.15 mmol) in DCM (80 mL) was added Ac₂O (1.41 g, 13.8 mmol) in dropwise at 0° C. under nitrogen. The mixture was stirred at room temperature for 4 h and concentrated under reduced pressure. The residue was diluted with EA (200 mL), washed with 1 N HCl (40 mL), saturated NaHCO₃ (60 mL) and brine (60 mL). The organic layer was dried over Na₂SO₄, filtered and concentrated under vacuum. The residue was purified by silica gel chromatograph (PE:EA=1:4) to give the title compound (4.5 g, 78%). LC-MS (M+H)⁺=501.1.

### Step 7: (5-{3-[3-(acetyloxy)-2,2-dimethylpropyl]-6-bromo-9-oxa-1-azatricyclo[6.3.1.0^{4,12}]dodeca-2,4(12),5,7-tetraen-2-yl}-6-[(1S)-1-methoxyethyl]pyridin-3-yl)boronic Acid

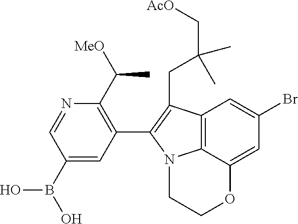 [SMILES: CO[C@@H](C)c1ncc(B(O)O)cc1-c1c(CC(C)(C)COC(C)=O)c2cc(Br)cc3c2n1CCO3]

To a mixture of 3-(6-bromo-2-{2-[(1S)-1-methoxyethyl]pyridin-3-yl}-9-oxa-1-azatricyclo[6.3.1.0^{4,12}]dodeca-2,4(12),5,7-tetraen-3-yl)-2,2-dimethylpropyl acetate (6.0 g, 12 mmol) and BPD (9.2 g, 72 mmol) in THF (80 mL) was added dtbpy (643 mg, 2.40 mmol) and chloro(1,5-cyclooctadiene)iridium(I) dimer (794 mg, 1.20 mmol). The mixture was stirred overnight at 75° C. under nitrogen and then cooled to room temperature. The mixture was diluted with EA (200 mL) and washed with water (50 mL) and brine (50 mL). The organic layer was dried over Na₂SO₄, filtered and concentrated under vacuum. The residue was purified by C18 chromatograph (MeCN:0.5% FA in water=0:100 to 55:45) to give the title compound (4.8 g, 73%). LC-MS (M+H)⁺=545.1/547.1.

### Step 8: 3-(6-bromo-2-{2-[(1S)-1-methoxyethyl]-5-(2,2,6,6-tetramethyloxan-4-yl)pyridin-3-yl}-9-oxa-1-azatricyclo[6.3.1.0^{4,12}]dodeca-2,4(12),5,7-tetraen-3-yl)-2,2-dimethylpropyl Acetate

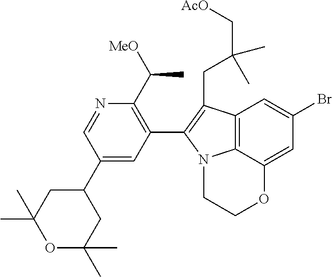 [SMILES: CO[C@@H](C)c1ncc(C2CC(C)(C)OC(C)(C)C2)cc1-c1c(CC(C)(C)COC(C)=O)c2cc(Br)cc3c2n1CCO3]

A mixture of (5-{3-[3-(acetyloxy)-2,2-dimethylpropyl]-6-bromo-9-oxa-1-azatricyclo[6.3.1.0^{4,12}]dodeca-2,4(12),5,7-tetraen-2-yl}-6-[(1S)-1-methoxyethyl]pyridin-3-yl)boronic acid (818 mg, 1.51 mmol), 4-methyl-N′-(2,2,6,6-tetramethyloxan-4-ylidene)benzene-1-sulfonohydrazide (1.5 g, 4.5 mmol) and Cs₂CO₃ (1.2 g, 3.8 mmol) in dioxane (30 mL) was stirred at 110° C. overnight under nitrogen. The mixture was cooled to room temperature and treated with brine (100 mL). The mixture was extracted with EA (50 mL×3). The organic layer was washed with brine (50 mL), dried over Na₂SO₄, filtered and concentrated. The residue was purified by silica gel chromatograph (DCM:MeOH=10:1) to give the title compound (420 mg, 43%). LC-MS (M+H)⁺=641.4/643.4.

### Step 9: 3-(2-{2-[(1S)-1-methoxyethyl]-5-(2,2,6,6-tetramethyloxan-4-yl)pyridin-3-yl}-6-(4,4,5,5-tetramethyl-1,3,2-dioxaborolan-2-yl)-9-oxa-1-azatricyclo[6.3.1.0^{4,12}]dodeca-2,4(12),5,7-tetraen-3-yl)-2,2-dimethylpropyl Acetate

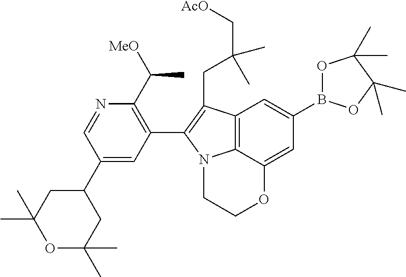

To a mixture of 3-(6-bromo-2-{2-[(1S)-1-methoxyethyl]-5-(2,2,6,6-tetramethyloxan-4-yl)pyridin-3-yl}-9-oxa-1-azatricyclo[6.3.1.0^{4,12}]dodeca-2,4(12),5,7-tetraen-3-yl)-2,2-dimethylpropyl acetate (420 mg, 0.65 mmol) and BPD (416 mg, 1.64 mmol) in dioxane (8 mL) was added Pd(dppf)Cl₂·DCM (53 mg, 0.065 mmol) and KOAc (191 mg, 1.95 mmol). The mixture was stirred for 12 h at 90° C. under nitrogen. The mixture was cooled to room temperature and diluted with EA (50 mL), washed with brine (40 mL). The organic phase was dried over Na₂SO₄, filtered and concentrated under vacuum. The residue was purified by silica gel chromatograph (DCM:MeOH=15:1) to give the title compound (310 mg, 69%). LC-MS (M+H)⁺=689.5.

### Step 10: methyl (4S)-2-[(2S)-3-(4-{3-[3-(acetyloxy)-2,2-dimethylpropyl]-2-{2-[(1S)-1-methoxyethyl]-5-(2,2,6,6-tetramethyloxan-4-yl)pyridin-3-yl}-9-oxa-1-azatricyclo[6.3.1.0^{4,12}]dodeca-2,4(12),5,7-tetraen-6-yl}-1,3-thiazol-2-yl)-2-{[(tert-butoxy)carbonyl]amino}propanoyl]-2,3-diazabicyclo[3.1.1]heptane-4-carboxylate

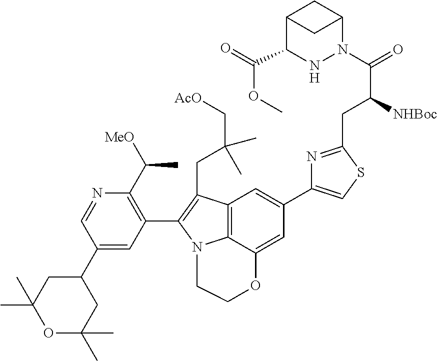 [SMILES: COC(=O)[C@H]1NN(C(=O)[C@H](Cc2nc(-c3cc4c5c(c3)c(CC(C)(C)COC(C)=O)c(-c3cc(C6CC(C)(C)OC(C)(C)C6)cnc3[C@H](C)OC)n5CCO4)cs2)NC(=O)OC(C)(C)C)C2CC1C2]

To a mixture of 3-(2-{2-[(1S)-1-methoxyethyl]-5-(2,2,6,6-tetramethyloxan-4-yl)pyridin-3-yl}-6-(4,4,5,5-tetramethyl-1,3,2-dioxaborolan-2-yl)-9-oxa-1-azatricyclo[6.3.1.0^{4,12}]dodeca-2,4(12),5,7-tetraen-3-yl)-2,2-dimethylpropyl acetate (282 mg, 0.41 mmol) and methyl (4S)-2-[(2S)-3-(4-bromo-1,3-thiazol-2-yl)-2-{[(tert-butoxy)carbonyl]amino}propanoyl]-2,3-diazabicyclo[3.1.1]heptane-4-carboxylate (200 mg, 0.41 mmol) in dioxane (6 mL) and water (0.6 mL) was added K₃PO₄ (260 mg, 1.23 mmol) and Pd(dtbpf)Cl₂ (27 mg, 0.041 mmol). The solution was stirred at 75° C. for 3 h under nitrogen. The mixture was cooled to room temperature, diluted with EA (40 mL) and washed with water (20 mL) and brine (20 mL). The organic layer was dried over Na₂SO₄, filtered and concentrated under vacuum. The residue was purified by silica gel chromatograph (DCM:MeOH=15:1) to give the title compound (330 mg, 83%). LC-MS (M+H)⁺=971.5.

### Step 11: (4S)-2-[(2S)-2-{[(tert-butoxy)carbonyl]amino}-3-{4-[3-(3-hydroxy-2,2-dimethylpropyl)-2-{2-[(1S)-1-methoxyethyl]-5-(2,2,6,6-tetramethyloxan-4-yl)pyridin-3-yl}-9-oxa-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic Acid

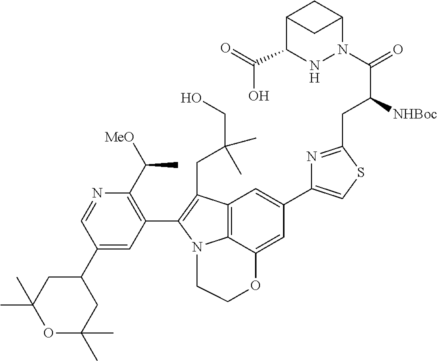 [SMILES: CO[C@@H](C)c1ncc(C2CC(C)(C)OC(C)(C)C2)cc1-c1c(CC(C)(C)CO)c2cc(-c3csc(C[C@H](NC(=O)OC(C)(C)C)C(=O)N4N[C@H](C(=O)O)C5CC4C5)n3)cc3c2n1CCO3]

To a mixture of methyl (4S)-2-[(2S)-3-(4-{3-[3-(acetyloxy)-2,2-dimethylpropyl]-2-{2-[(1S)-1-methoxyethyl]-5-(2,2,6,6-tetramethyloxan-4-yl)pyridin-3-yl}-9-oxa-1-azatricyclo[6.3.1.0^{4,12}]dodeca-2,4(12),5,7-tetraen-6-yl}-1,3-thiazol-2-yl)-2-{[(tert-butoxy)carbonyl]amino}propanoyl]-2,3-diazabicyclo[3.1.1]heptane-4-carboxylate (330 mg, 0.34 mmol) in THF (3 mL) and water (3 mL) was added LiOH (57 mg, 2.4 mmol). The mixture was stirred for 5 h at room temperature. Volatiles were removed under vacuum and the residue was diluted with water (40 mL). The mixture was washed with MTBE (2×10 mL). The organic layer was discarded and the pH of the aqueous layer was adjusted to ˜5 with 1 N hydrochloric acid at 0° C. The solution was extracted with EA (30 mL×3). The combined organic layer was dried over Na₂SO₄, filtered and concentrated under vacuum to give the title compound (200 mg, 64%). LC-MS (M+H)⁺=915.5.

### Step 12: tert-butyl N-[(7S,13S)-20-{2-[(1S)-1-methoxyethyl]-5-(2,2,6,6-tetramethyloxan-4-yl)pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate

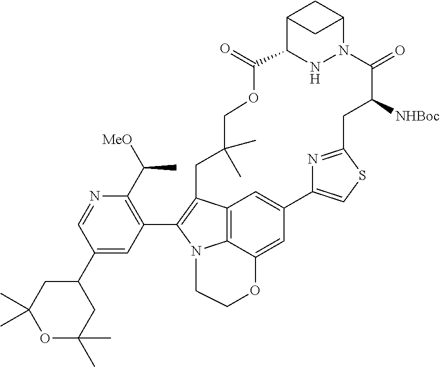 [SMILES: CO[C@@H](C)c1ncc(C2CC(C)(C)OC(C)(C)C2)cc1-c1c2c3cc(cc4c3n1CCO4)-c1csc(n1)C[C@H](NC(=O)OC(C)(C)C)C(=O)N1N[C@H](C(=O)OCC(C)(C)C2)C2CC1C2]

To a mixture of (4S)-2-[(2S)-2-{[(tert-butoxy)carbonyl]amino}-3-{4-[3-(3-hydroxy-2,2-dimethylpropyl)-2-{2-[(1S)-1-methoxyethyl]-5-(2,2,6,6-tetramethyloxan-4-yl)pyridin-3-yl}-9-oxa-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic acid (200 mg, 0.22 mmol) in MeCN (20 mL) was added TCFH (1.84 g, 6.55 mmol) and NMI (537 mg, 6.55 mmol). The mixture was stirred at room temperature for 2 h under nitrogen. The mixture was concentrated under vacuum, and the residue was dissolved in EA (40 mL). The mixture was washed with water (15 mL) and brine (15 mL), dried over Na₂SO₄, filtered and concentrated under vacuum. The residue was purified by C18 chromatograph (MeCN:0.5% FA in water=9:1) to give the title compound (80 mg, 41%). LC-MS (M+H)⁺=897.5.

### Step 13: (7S,13S)-7-amino-20-{2-[(1S)-1-methoxyethyl]-5-(2,2,6,6-tetramethyloxan-4-yl)pyridin-3-yl}-17,17-dimethyl-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione

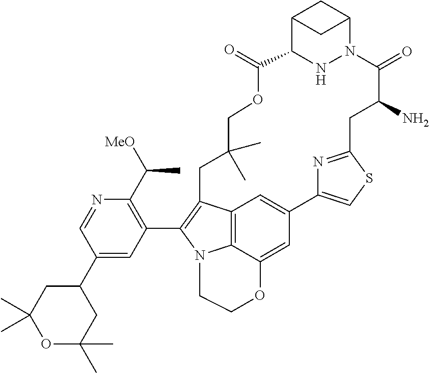 [SMILES: CO[C@@H](C)c1ncc(C2CC(C)(C)OC(C)(C)C2)cc1-c1c2c3cc(cc4c3n1CCO4)-c1csc(n1)C[C@H](N)C(=O)N1N[C@H](C(=O)OCC(C)(C)C2)C2CC1C2]

A mixture of tert-butyl N-[(7S,13S)-20-{2-[(1S)-1-methoxyethyl]-5-(2,2,6,6-tetramethyloxan-4-yl)pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate (80 mg, 0.089 mmol) in HCl (4 M in dioxane, 3 mL) was stirred for 1 h at room temperature and concentrated under vacuum. The residue was partitioned between DCM (10 mL) and saturated NaHCO₃ (5 mL). The organic layer was dried over Na₂SO₄, filtered and concentrated under vacuum to give the title compound (60 mg, 85%). LC-MS (M+H)⁺=797.5.

### Step 14: (1R,2R,3S)—N-[(7S,13S,19P)-20-{2-[(1S)-1-methoxyethyl]-5-(2,2,6,6-tetramethyloxan-4-yl)pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide & (1R,2R,3S)—N-[(7S,13S,19M)-20-{2-[(1S)-1-methoxyethyl]-5-(2,2,6,6-tetramethyloxan-4-yl)pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

To a solution of (7S,13S)-7-amino-20-{2-[(1S)-1-methoxyethyl]-5-(2,2,6,6-tetramethyloxan-4-yl)pyridin-3-yl}-17,17-dimethyl-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione (48 mg, 0.06 mmol) in DMF (3 mL) was added DIPEA (387 mg, 3.0 mmol), (1r,2R,3S)-2,3-dimethylcyclopropane-1-carboxylic acid (14 mg, 0.12 mmol) and HATU (46 mg, 0.12 mmol). The mixture was stirred for 1 h at room temperature and then diluted with EA (15 mL). The mixture was washed with brine (10 mL), dried over Na₂SO₄, filtered and concentrated under vacuum. The residue was purified by prep-TLC (DCM:MeOH=10:1) followed by prep-HPLC to give Atrop-5 (P-isomer, 2.4 mg, 5%) and example 5 (M-isomer, 16 mg, 30%).

Atrop-5: ¹H NMR (400 MHz, DMSO-d6) δ 8.62 (s, 1H), 8.39 (d, J=8.0 Hz, 1H), 8.03 (s, 1H), 7.79-7.71 (m, 2H), 7.13 (s, 1H), 5.83 (d, J=12.0 Hz, 1H), 5.30-5.22 (m, 1H), 4.69 (d, J=12.0 Hz, 1H), 4.54-4.35 (m, 3H), 4.15-4.06 (m, 1H), 4.03-3.95 (m, 1H), 3.67-3.60 (m, 1H), 3.59-3.54 (m, 1H), 3.49-3.43 (m, 1H), 3.26-3.15 (m, 3H), 3.10 (s, 3H), 3.08-3.05 (m, 1H), 3.00-2.93 (m, 1H), 2.65-2.60 (m, 1H), 2.34-2.26 (m, 2H), 2.12-2.04 (m, 1H), 1.68-1.54 (m, 3H), 1.45-1.36 (m, 2H), 1.20-1.16 (m, 1H), 1.14-1.08 (m, 11H), 1.06-1.00 (m, 6H), 0.88 (s, 3H), 0.38 (s, 3H). LC-MS (M+H)⁺=893.5.

Example 5: ¹H NMR (500 MHz, DMSO-d6) δ 8.67 (d, J=2.2 Hz, 1H), 8.42 (d, J=8.8 Hz, 1H), 8.06 (d, J=1.2 Hz, 1H), 7.78 (s, 1H), 7.65 (d, J=2.3 Hz, 1H), 7.18 (d, J=1.1 Hz, 1H), 5.91 (d, J=11.0 Hz, 1H), 5.36 (t, J=8.3 Hz, 1H), 4.70 (d, J=11.0 Hz, 1H), 4.66-4.55 (m, 2H), 4.50 (q, J=4.9 Hz, 1H), 4.44 (q, J=6.1 Hz, 1H), 4.32-4.22 (m, 1H), 3.72 (ddd, J=12.4, 8.2, 4.0 Hz, 1H), 3.64-3.49 (m, 2H), 3.33-3.19 (m, 5H), 3.13 (dd, J=14.9, 7.4 Hz, 1H), 2.95 (d, J=14.3 Hz, 1H), 2.71-2.61 (m, 1H), 2.50-2.43 (m, 2H), 2.39-2.28 (m, 1H), 2.13 (t, J=9.8 Hz, 1H), 1.85-1.66 (m, 2H), 1.58 (t, J=9.4 Hz, 1H), 1.51-1.40 (m, 2H), 1.37 (d, J=6.0 Hz, 3H), 1.32-1.28 (m, 6H), 1.27-1.13 (m, 9H), 1.09 (d, J=6.3 Hz, 3H), 1.07 (d, J=5.5 Hz, 3H), 0.91 (s, 3H), 0.34 (s, 3H). LC-MS (M+H)⁺=893.5.

Atrop-6 & Example 6: (1R,2R,3S)—N-[(7S,13S,19P)-20-{2-[(1S)-1-methoxyethyl]-5-(2,2,6,6-tetramethyloxan-4-yl)pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide & (1R,2R,3S)—N-[(7S,13S,19M)-20-{2-[(1S)-1-methoxyethyl]-5-(2,2,6,6-tetramethyloxan-4-yl)pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

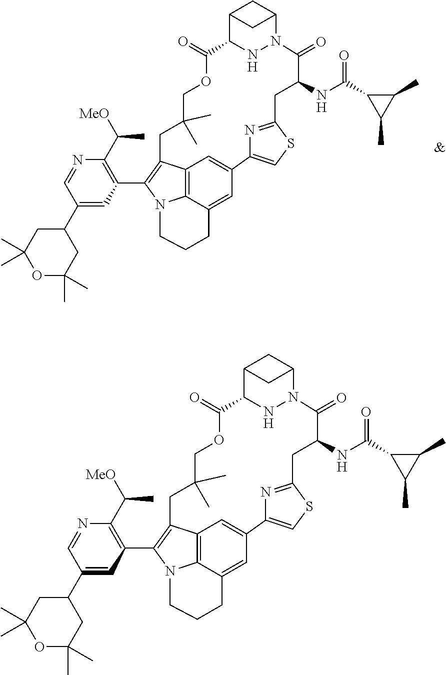

### Step 1: 6-bromo-1,2,3,4-tetrahydroquinolin-1-amine

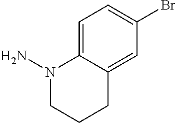 [SMILES: NN1CCCc2cc(Br)ccc21]

To a solution of 6-bromo-1,2,3,4-tetrahydroquinoline (87.3 g, 412 mmol) in EtOH (900 mL) was added a solution of NaNO₂ (56.8 g, 823 mmol) in water (540 mL). The mixture was cooled to 0° C., and concentrated hydrochloric acid (190 mL) was dropwise added at 0° C. with vigorously stirring. The mixture was stirred for 2 h at 0° C. The reaction mixture was diluted with water (500 mL) and extracted with EA (500 mL×3). The combined organic layer was washed with brine (500 mL×3), dried over Na₂SO₄, filtered and concentrated under vacuum to give a residue. The reside was dissolved in THF (500 mL) and H₂O (500 mL) followed by the addition of NH₄Cl (229 g, 4.28 mol) and zinc powder (93.3 g, 1.43 mol) at 0° C. The reaction mixture was stirred at 0° C. for 2 h. Solids were filtered off, and the filtrate was extracted with EA (500 mL×3). The combined organic layer was washed with brine (500 mL×3), dried over Na₂SO₄, filtered and concentrated under vacuum to give a residue. The residue was purified by silica gel chromatograph (PE:EA=10:1 to 1:1) to give the title compound (53.6 g, 57%). ¹H NMR (400 MHz, DMSO-d6) δ=7.11-7.06 (m, 2H), 7.01-6.97 (m, 1H), 4.33-4.20 (m, 2H), 3.25-3.21 (m, 2H), 2.66-2.61 (m, 2H), 1.92-1.86 (m, 2H). LC-MS (M+H)⁺=226.9/229.0.

### Step 2: 3-(6-bromo-2-{2-[(1S)-1-methoxyethyl]pyridin-3-yl}-1-azatricyclo[6.3.1.0^{4,12}]dodeca-2,4(12),5,7-tetraen-3-yl)-2,2-dimethylpropanoic Acid

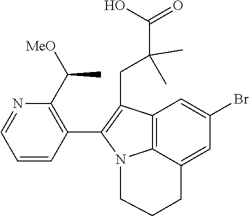 [SMILES: CO[C@@H](C)c1ncccc1-c1c(CC(C)(C)C(=O)O)c2cc(Br)cc3c2n1CCC3]

To a mixture of 6-bromo-1,2,3,4-tetrahydroquinolin-1-amine (2.62 g, 11.7 mmol) and 5-{2-[(1S)-1-methoxyethyl]pyridin-3-yl}-2,2-dimethyl-5-oxopentanoic acid (3.1 g, 11 mmol) in toluene (150 mL) was added TsOH·H₂O (4.2 g, 22 mmol). The mixture was stirred at 100° C. for 16 h under nitrogen and then cooled to room temperature. The mixture was concentrated under vacuum. The residue was dissolved in EA (200 mL) and successively washed with water (80 mL) and brine (80 mL). The organic phase was dried over Na₂SO₄, filtered and concentrated under vacuum to give the title compound (5.2 g, 100%). LC-MS (M+H)⁺=471.1/473.1.

### Step 3: ethyl 3-(6-bromo-2-{2-[(1S)-1-methoxyethyl]pyridin-3-yl}-1-azatricyclo[6.3.1.0^{4,12}]dodeca-2,4(12),5,7-tetraen-3-yl)-2,2-dimethylpropanoate

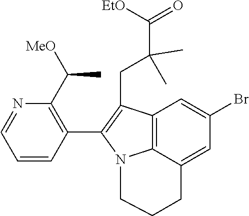 [SMILES: CCOC(=O)C(C)(C)Cc1c(-c2cccnc2[C@H](C)OC)n2c3c(cc(Br)cc13)CCC2]

To a solution of 3-(6-bromo-2-(2-[(1S)-1-methoxyethyl]pyridin-3-yl)-1-azatricyclo[6.3.1.0^{4,12}]dodeca-2,4(12),5,7-tetraen-3-yl)-2,2-dimethylpropanoic acid (5.2 g, 11.1 mmol) in DMF (50 mL) was added Cs₂CO₃ (5.38 g, 16.5 mmol), and then EtI (2.57 g, 16.5 mmol) was added dropwise at 0° C. under nitrogen. The mixture was stirred at room temperature for 2 h, diluted with EA (200 mL) and washed with brine (80 mL×2). The organic phase was dried over Na₂SO₄, filtered and concentrated under vacuum to give a residue. The residue was purified by silica gel chromatograph (PE:EA=1:4) to give the title compound (5.1 g, 92%). LC-MS (M+H)⁺=499.3/501.3.

### Step 4: 3-(6-bromo-2-{2-[(1S)-1-methoxyethyl]pyridin-3-yl}-1-azatricyclo[6.3.1.0^{4,12}]dodeca-2,4(12),5,7-tetraen-3-yl)-2,2-dimethylpropan-1-ol

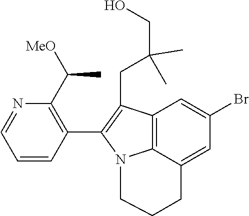 [SMILES: CO[C@@H](C)c1ncccc1-c1c(CC(C)(C)CO)c2cc(Br)cc3c2n1CCC3]

To a solution of ethyl 3-(6-bromo-2-{2-[(1S)-1-methoxyethyl]pyridin-3-yl}-1-azatricyclo[6.3.1.0^{4,12}]dodeca-2,4(12),5,7-tetraen-3-yl)-2,2-dimethylpropanoate (5.1 g, 10.2 mmol) in THF (80 mL) at 0° C. was added LiBH₄ (536 mg, 25.5 mmol) in portions under nitrogen. The mixture was stirred at 60° C. for 16 h. The mixture was cooled to room temperature followed by slow addition of pre-cooled (0° C.) aqueous solution of NH₄Cl (100 mL). The mixture was extracted with EA (150 mL×2). The combined organic layer was washed with brine (80 mL), dried over Na₂SO₄, filtered and concentrated under vacuum to give the title compound (4.6 g, 98%). LC-MS (M+H)⁺=457.3/459.3.

### Step 5: 3-(6-bromo-2-{2-[(1S)-1-methoxyethyl]pyridin-3-yl}-1-azatricyclo[6.3.1.0^{4,12}]dodeca-2,4(12),5,7-tetraen-3-yl)-2,2-dimethylpropyl Acetate

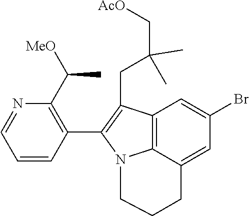 [SMILES: CO[C@@H](C)c1ncccc1-c1c(CC(C)(C)COC(C)=O)c2cc(Br)cc3c2n1CCC3]

To a mixture of 3-(6-bromo-2-{2-[(1S)-1-methoxyethyl]pyridin-3-yl}-1-azatricyclo[6.3.1.0^{4,12}]dodeca-2,4(12),5,7-tetraen-3-yl)-2,2-dimethylpropan-1-ol (4.6, 10.0 mmol) and Et₃N (2.06 g, 20.4 mmol) in DCM (70 mL) was added DMAP (124 mg, 1.02 mmol) and Ac₂O (1.25 g, 12.2 mmol) in portions at 0° C. under nitrogen. The mixture was stirred for 3 h at room temperature and concentrated under reduced pressure. The residue was diluted with DCM (60 mL), washed with hydrochloric acid (1 N, 40 mL), sat. NaHCO₃ (60 mL) and brine (60 mL). The organic layer was dried over Na₂SO₄, filtered and concentrated under vacuum. The residue was purified by silica gel chromatograph (PE:EA=1:4) to give the title compound (4.1 g, 82%). LC-MS (M+H)⁺=499.3/501.3.

### Step 6: (5-{3-[3-(acetyloxy)-2,2-dimethylpropyl]-6-bromo-1-azatricyclo[6.3.1.0^{4,12}]dodeca-2,4(12),5,7-tetraen-2-yl}-6-[(1S)-1-methoxyethyl]pyridin-3-yl)boronic Acid

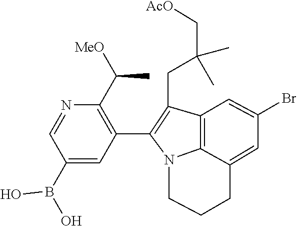 [SMILES: CO[C@@H](C)c1ncc(B(O)O)cc1-c1c(CC(C)(C)COC(C)=O)c2cc(Br)cc3c2n1CCC3]

The title compound (8.1 g, 32%) was prepared in a manner similar to that in example 1 step 9 from 3-(6-bromo-2-{2-[(1S)-1-methoxyethyl]pyridin-3-yl}-1-azatricyclo[6.3.1.0^{4,12}]dodeca-2,4(12),5,7-tetraen-3-yl)-2,2-dimethylpropyl acetate and BPD. LC-MS (M+H)⁺=543.3/545.3.

### Step 7: 3-(6-bromo-2-{2-[(1S)-1-methoxyethyl]-5-(2,2,6,6-tetramethyloxan-4-yl)pyridin-3-yl}-1-azatricyclo[6.3.1.0^{4,12}]dodeca-2,4(12),5,7-tetraen-3-yl)-2,2-dimethylpropyl Acetate

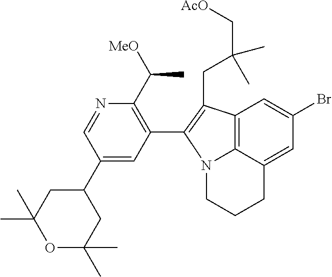 [SMILES: CO[C@@H](C)c1ncc(C2CC(C)(C)OC(C)(C)C2)cc1-c1c(CC(C)(C)COC(C)=O)c2cc(Br)cc3c2n1CCC3]

The title compound (150 mg, 35%) was prepared in a manner similar to that in example 5 step 8 from (5-{3-[3-(acetyloxy)-2,2-dimethylpropyl]-6-bromo-1-azatricyclo[6.3.1.0^{4,12}]dodeca-2,4(12),5,7-tetraen-2-yl}-6-[(1S)-1-methoxyethyl]pyridin-3-yl)boronic acid and 4-methyl-N′-(2,2,6,6-tetramethyloxan-4-ylidene)benzene-1-sulfonohydrazide. LC-MS (M+H)⁺=639.3/641.3.

### Step 8: 3-(2-{2-[(1S)-1-methoxyethyl]-5-(2,2,6,6-tetramethyloxan-4-yl)pyridin-3-yl}-6-(4,4,5,5-tetramethyl-1,3,2-dioxaborolan-2-yl)-1-azatricyclo[6.3.1.0^{4,12}]dodeca-2,4(12),5,7-tetraen-3-yl)-2,2-dimethylpropyl Acetate

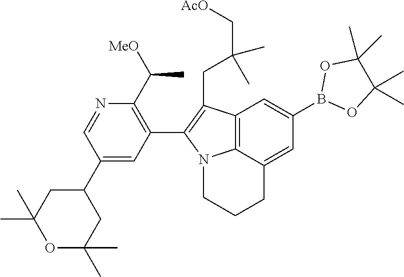

The title compound (110 mg, 68%) was prepared in a manner similar to that in example 5 step 9 from 3-(6-bromo-2-{2-[(1S)-1-methoxyethyl]-5-(2,2,6,6-tetramethyloxan-4-yl)pyridin-3-yl}-1-azatricyclo[6.3.1.0^{4,12}]dodeca-2,4(12),5,7-tetraen-3-yl)-2,2-dimethylpropyl acetate. LC-MS (M+H)⁺=687.5.

### Step 9: methyl (4S)-2-[(2S)-3-(4-{3-[3-(acetyloxy)-2,2-dimethylpropyl]-2-{2-[(1S)-1-methoxyethyl]-5-(2,2,6,6-tetramethyloxan-4-yl)pyridin-3-yl}-1-azatricyclo[6.3.1.0^{4,12}]dodeca-2,4(12),5,7-tetraen-6-yl}-1,3-thiazol-2-yl)-2-{[(tert-butoxy)carbonyl]amino}propanoyl]-2,3-diazabicyclo[3.1.1]heptane-4-carboxylate

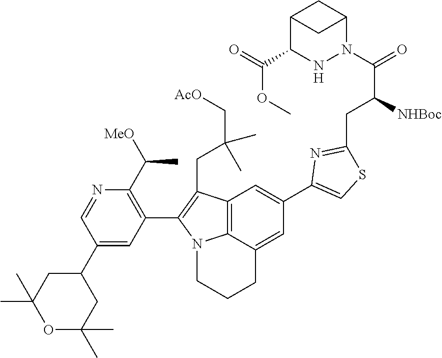 [SMILES: COC(=O)[C@H]1NN(C(=O)[C@H](Cc2nc(-c3cc4c5c(c3)c(CC(C)(C)COC(C)=O)c(-c3cc(C6CC(C)(C)OC(C)(C)C6)cnc3[C@H](C)OC)n5CCC4)cs2)NC(=O)OC(C)(C)C)C2CC1C2]

The title compound (120 mg, 77%) was prepared in a manner similar to that in example 5 step 10 from 3-(2-{2-[(1S)-1-methoxyethyl]-5-(2,2,6,6-tetramethyloxan-4-yl)pyridin-3-yl}-6-(4,4,5,5-tetramethyl-1,3,2-dioxaborolan-2-yl)-1-azatricyclo[6.3.1.0^{4,12}]dodeca-2,4(12),5,7-tetraen-3-yl)-2,2-dimethylpropyl acetate and methyl (4S)-2-[(2S)-3-(4-bromo-1,3-thiazol-2-yl)-2-{[(tert-butoxy)carbonyl]amino}propanoyl]-2,3-diazabicyclo[3.1.1]heptane-4-carboxylate. LC-MS (M+H)⁺=969.6.

### Step 10: (4S)-2-[(2S)-2-{[(tert-butoxy)carbonyl]amino}-3-{4-[3-(3-hydroxy-2,2-dimethylpropyl)-2-{2-[(1S)-1-methoxyethyl]-5-(2,2,6,6-tetramethyloxan-4-yl)pyridin-3-yl}-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic Acid

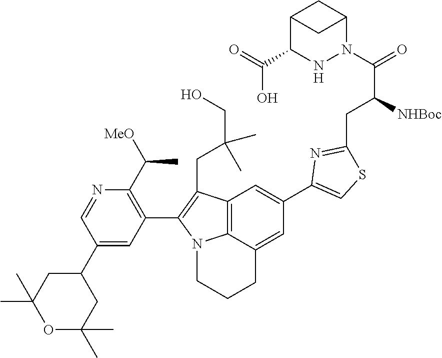 [SMILES: CO[C@@H](C)c1ncc(C2CC(C)(C)OC(C)(C)C2)cc1-c1c(CC(C)(C)CO)c2cc(-c3csc(C[C@H](NC(=O)OC(C)(C)C)C(=O)N4N[C@H](C(=O)O)C5CC4C5)n3)cc3c2n1CCC3]

The title compound (60 mg, 53%) was prepared in a manner similar to that in example 5 step 11 from methyl (4S)-2-[(2S)-3-(4-{3-[3-(acetyloxy)-2,2-dimethylpropyl]-2-{2-[(1S)-1-methoxyethyl]-5-(2,2,6,6-tetramethyloxan-4-yl)pyridin-3-yl}-1-azatricyclo[6.3.1.0^{4,12}]dodeca-2,4(12),5,7-tetraen-6-yl}-1,3-thiazol-2-yl)-2-{[(tert-butoxy)carbonyl]amino}propanoyl]-2,3-diazabicyclo[3.1.1]heptane-4-carboxylate. LC-MS (M+H)⁺=913.6.

### Step 11: tert-butyl N-[(7S,13S)-20-{2-[(1S)-1-methoxyethyl]-5-(2,2,6,6-tetramethyloxan-4-yl)pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate

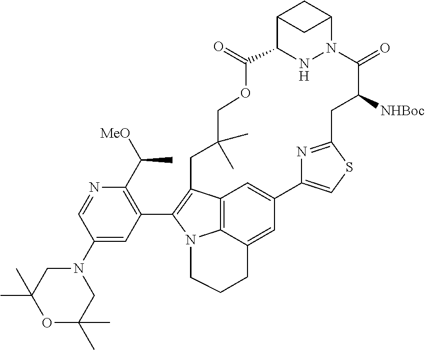 [SMILES: CO[C@@H](C)c1ncc(N2CC(C)(C)OC(C)(C)C2)cc1-c1c2c3cc(cc4c3n1CCC4)-c1csc(n1)C[C@H](NC(=O)OC(C)(C)C)C(=O)N1N[C@H](C(=O)OCC(C)(C)C2)C2CC1C2]

The title compound (30 mg, 51%) was prepared in a manner similar to that in example 5 step 12 from (4S)-2-[(2S)-2-{[(tert-butoxy)carbonyl]amino}-3-{4-[3-(3-hydroxy-2,2-dimethylpropyl)-2-{2-[(1S)-1-methoxyethyl]-5-(2,2,6,6-tetramethyloxan-4-yl)pyridin-3-yl}-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic acid. LC-MS (M+H)⁺=895.6.

### Step 12: (7S,13S)-7-amino-20-{2-[(1S)-1-methoxyethyl]-5-(2,2,6,6-tetramethyloxan-4-yl)pyridin-3-yl}-17,17-dimethyl-15-oxa-4-thia-9,21,30,32-tetraazaheptacyclo[23.3.1.1^{2,5}.1^{10,12}.0^{19,27}.0^{21,26}]dotriaconta-1(28),2,5(32),19,25(29),26-hexaene-8,14-dione

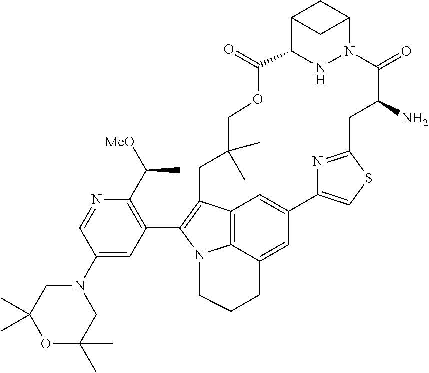 [SMILES: CO[C@@H](C)c1ncc(N2CC(C)(C)OC(C)(C)C2)cc1-c1c2c3cc(cc4c3n1CCC4)-c1csc(n1)C[C@H](N)C(=O)N1N[C@H](C(=O)OCC(C)(C)C2)C2CC1C2]

The title compound (25 mg, 94%) was prepared in a manner similar to that in example 5 step 13 from tert-butyl N-[(7S,13S)-20-{2-[(1S)-1-methoxyethyl]-5-(2,2,6,6-tetramethyloxan-4-yl)pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate. LC-MS (M+H)⁺=795.6.

### Step 13: (1R,2R,3S)—N-[(7S,13S,19P)-20-{2-[(1S)-1-methoxyethyl]-5-(2,2,6,6-tetramethyloxan-4-yl)pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide & (1R,2R,3S)—N-[(7S,13S,19M)-20-{2-[(1S)-1-methoxyethyl]-5-(2,2,6,6-tetramethyloxan-4-yl)pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

Atrop-6 (P-isomer, 2.8 mg, 10%) and example 6 (M-isomer, 8 mg, 29%) were prepared in a manner similar to that in example 5 step 14 from (7S,13S)-7-amino-20-{2-[(1S)-1-methoxyethyl]-5-(2,2,6,6-tetramethyloxan-4-yl)pyridin-3-yl}-17,17-dimethyl-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione and (1r,2R,3S)-2,3-dimethylcyclopropane-1-carboxylic acid.

Atrop-6: ¹H NMR (400 MHz, DMSO-d6) δ 8.62 (d, J=4.0 Hz, 1H), 8.39 (d, J=8.0 Hz, 1H), 8.22 (s, 1H), 7.72 (s, 1H), 7.69 (s, 1H), 7.40 (s, 1H), 5.84 (d, J=12.0 Hz, 1H), 5.30-5.22 (m, 1H), 4.70 (d, J=12.0 Hz, 1H), 4.49-4.42 (m, 1H), 4.07-3.99 (m, 1H), 3.88-3.79 (m, 1H), 3.59-3.54 (m, 1H), 3.49-3.43 (m, 1H), 3.45-3.39 (m, 1H), 3.27-3.22 (m, 3H), 3.21-3.15 (m, 1H), 3.14-3.09 (m, 1H), 3.01-2.90 (m, 3H), 2.65-2.60 (m, 1H), 2.36-2.24 (m, 3H), 2.21-2.14 (m, 1H), 2.12-2.05 (m, 1H), 2.02-1.95 (m, 1H), 1.69-1.62 (m, 2H), 1.59-1.54 (m, 1H), 1.45-1.37 (m, 2H), 1.26 (s, 6H), 1.21-1.18 (m, 1H), 1.15-1.07 (m, 12H), 1.06-1.00 (m, 6H), 0.87 (s, 3H), 0.40 (s, 3H). LC-MS (M+H)⁺=891.6.

Example 6: ¹H NMR (400 MHz, DMSO-d6) δ 8.67 (d, J=2.3 Hz, 1H), 8.41 (d, J=8.9 Hz, 1H), 8.26 (s, 1H), 7.73 (s, 1H), 7.60 (d, J=2.3 Hz, 1H), 7.45 (s, 1H), 5.93 (d, J=11.0 Hz, 1H), 5.56-5.28 (m, 1H), 4.66 (d, J=11.0 Hz, 1H), 4.56-4.45 (m, 1H), 4.43-4.29 (m, 1H), 4.21-4.14 (m, 1H), 3.70-3.49 (m, 3H), 3.32-3.09 (m, 6H), 3.10-2.98 (m, 1H), 2.97-2.84 (m, 2H), 2.75-2.59 (m, 2H), 2.48-2.08 (m, 5H), 1.88-1.67 (m, 2H), 1.62-1.39 (m, 3H), 1.37 (d, J=6.0 Hz, 3H), 1.33-1.26 (m, 6H), 1.27-1.12 (m, 9H), 1.09 (d, J=6.1 Hz, 3H), 1.06 (d, J=5.3 Hz, 3H), 0.86 (s, 3H), 0.37 (s, 3H). LC-MS (M+H)⁺=891.6.

### Example 7 & Example 8: (1R,2R,3S)—N-[(7S,13S,19M)-20-{5-[(2R,6S)-2,6-dimethylmorpholin-4-yl]-2-[(1S)-1-methoxyethyl]pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide & (1S,2S)—N-[(7S,13S,19M)-20-{5-[(2R,6S)-2,6-dimethylmorpholin-4-yl]-2-[(1S)-1-methoxyethyl]pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2-methylcyclopropane-1-carboxamide

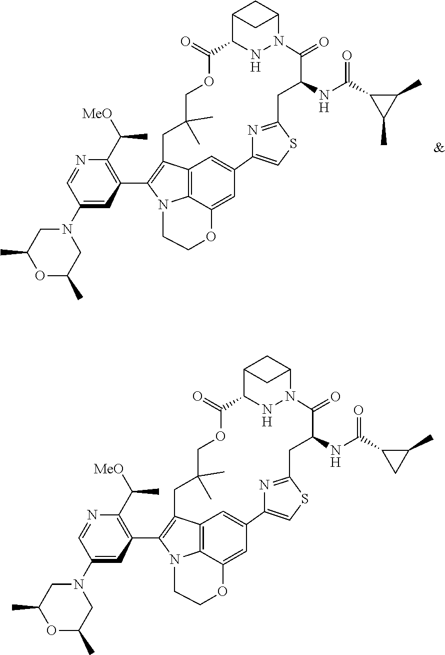

### Step 1: 3-(6-bromo-2-{2-[(S)-1-methoxyethyl]-5-(4,4,5,5-tetramethyl-1,3,2-dioxaborolan-2-yl)pyridin-3-yl}-9-oxa-1-azatricyclo[6.3.1.0^{4,12}]dodeca-2,4(12),5,7-tetraen-3-yl)-2,2-dimethylpropyl Acetate

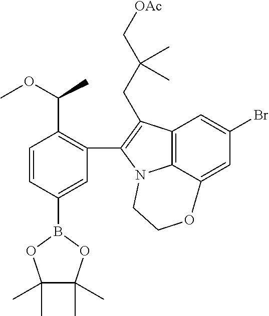 [SMILES: CO[C@@H](C)c1ccc(B2OC(C)(C)C(C)(C)O2)cc1-c1c(CC(C)(C)COC(C)=O)c2cc(Br)cc3c2n1CCO3]

To a mixture of 3-(6-bromo-2-(2-[(1S)-1-methoxyethyl]pyridin-3-yl)-9-oxa-1-azatricyclo[6.3.1.0^{4,12}]dodeca-2,4(12),5,7-tetraen-3-yl)-2,2-dimethylpropyl acetate (2.0 g, 3.98 mmol) and BPD (1.72 g, 6.8 mmol) in THF (40 mL) was added dtbpy (182 mg, 0.68 mmol) and chloro(1,5-cyclooctadiene)iridium(I) dimer (188 mg, 0.28 mmol). The mixture was stirred at 75° C. under nitrogen for overnight then cooled to room temperature. The mixture was diluted with EA (100 mL), successively washed with water (40 mL) and brine (30 mL). The organic layer was dried over Na₂SO₄, filtered and concentrated under vacuum. The residue was purified by silica gel chromatograph (DCM:MeOH=20:1) to give the title compound (1.9 g, 76%). LC-MS (M-pinacol+2H₂O+H)⁺=545.1.

### Step 2: 3-(6-bromo-2-{5-[(2R,6S)-2,6-dimethylmorpholin-4-yl]-2-[(1S)-1-methoxyethyl]pyridin-3-yl}-9-oxa-1-azatricyclo[6.3.1.0^{4,12}]dodeca-2,4(12),5,7-tetraen-3-yl)-2,2-dimethylpropyl Acetate

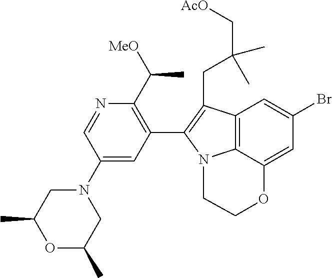 [SMILES: CO[C@@H](C)c1ncc(N2C[C@@H](C)O[C@@H](C)C2)cc1-c1c(CC(C)(C)COC(C)=O)c2cc(Br)cc3c2n1CCO3]

To a mixture of 3-(6-bromo-2-{2-[(1S)-1-methoxyethyl]-5-(4,4,5,5-tetramethyl-1,3,2-dioxaborolan-2-yl)pyridin-3-yl}-9-oxa-1-azatricyclo[6.3.1.0^{4,12}]dodeca-2,4(12),5,7-tetraen-3-yl)-2,2-dimethylpropyl acetate (450 mg, 0.72 mmol) and (2R,6S)-2,6-dimethylmorpholine (413 mg, 3.59 mmol) in MeCN (15 mL) was added Et₃N (218 mg, 2.16 mmol) and Cu(OAc)₂ (262 mg, 1.44 mmol). The mixture was stirred for 4 h at room temperature under oxygen. The mixture was diluted with EA (50 mL) and water (50 mL). Solid was filtered off and the filter cake was rinsed with EA (50 mL×2). The organic phase of the filtrate was separated, successively washed with water (50 mL) and brine (50 mL), dried over Na₂SO₄, filtered and concentrated under vacuum. The residue was purified by silica gel column chromatography (DCM:MeOH=20:1) to give the title compound (270 mg, 61%). LC-MS (M+H)⁺=614.3.

### Step 3: 3-(2-{5-[(2R,6S)-2,6-dimethylmorpholin-4-yl]-2-[(1S)-1-methoxyethyl]pyridin-3-yl}-6-(4,4,5,5-tetramethyl-1,3,2-dioxaborolan-2-yl)-9-oxa-1-azatricyclo[6.3.1.0^{4,12}]dodeca-2,4(12),5,7-tetraen-3-yl)-2,2-dimethylpropyl Acetate

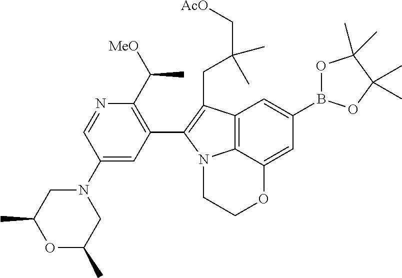

To a mixture of 3-(6-bromo-2-{5-[(2R,6S)-2,6-dimethylmorpholin-4-yl]-2-[(1S)-1-methoxyethyl]pyridin-3-yl}-9-oxa-1-azatricyclo[6.3.1.0^{4,12}]dodeca-2,4(12),5,7-tetraen-3-yl)-2,2-dimethylpropyl acetate (270 mg, 0.44 mmol) and BPD (224 mg, 0.88 mmol) in dioxane (8 mL) was added Pd(dppf)Cl₂·DCM (36 mg, 0.044 mmol) and KOAc (129 mg, 1.32 mmol). The mixture was stirred for 12 h at 100° C. under nitrogen. The mixture was cooled to room temperature and diluted with EA (50 mL), washed with brine (40 mL). The organic phase was dried over Na₂SO₄, filtered and concentrated under vacuum. The residue was purified by silica gel chromatograph (DCM:MeOH=15:1) to give the title compound (160 mg, 55%). LC-MS (M+H)⁺=662.5.

### Step 4: methyl (4S)-2-[(2S)-3-(4-{3-[3-(acetyloxy)-2,2-dimethylpropyl]-2-{5-[(2R,6S)-2,6-dimethylmorpholin-4-yl]-2-[(1S)-1-methoxyethyl]pyridin-3-yl}-9-oxa-1-azatricyclo[6.3.1.0^{4,12}]dodeca-2,4(12),5,7-tetraen-6-yl}-1,3-thiazol-2-yl)-2-{[(tert-butoxy)carbonyl]amino}propanoyl]-2,3-diazabicyclo[3.1.1]heptane-4-carboxylate

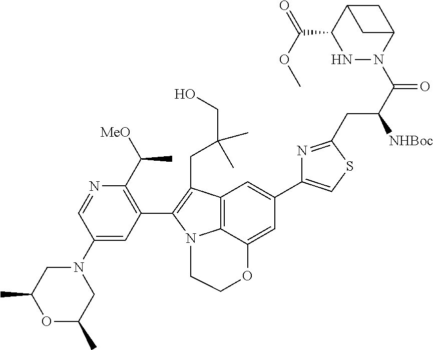 [SMILES: COC(=O)[C@H]1NN(C(=O)[C@H](Cc2nc(-c3cc4c5c(c3)c(CC(C)(C)CO)c(-c3cc(N6C[C@@H](C)O[C@@H](C)C6)cnc3[C@H](C)OC)n5CCO4)cs2)NC(=O)OC(C)(C)C)C2CC1C2]

To a mixture of 3-(2-{5-[(2R,6S)-2,6-dimethylmorpholin-4-yl]-2-[(1S)-1-methoxyethyl]pyridin-3-yl}-6-(4,4,5,5-tetramethyl-1,3,2-dioxaborolan-2-yl)-9-oxa-1-azatricyclo[6.3.1.0^{4,12}]dodeca-2,4(12),5,7-tetraen-3-yl)-2,2-dimethylpropyl acetate (271 mg, 0.41 mmol) and methyl (4S)-2-[(2S)-3-(4-bromo-1,3-thiazol-2-yl)-2-{[(tert-butoxy)carbonyl]amino}propanoyl]-2,3-diazabicyclo[3.1.1]heptane-4-carboxylate (200 mg, 0.41 mmol) in dioxane (10 mL) and water (1 mL) was added K₃PO₄ (260 mg, 1.23 mmol) and Pd(dtbpf)Cl₂ (27 mg, 0.041 mmol). The mixture was stirred for 3 h at 75° C. under nitrogen and then cooled to room temperature. The mixture was diluted with EA (50 mL), successively washed with water (50 mL), brine (50 mL), dried over Na₂SO₄, filtered and concentrated under vacuum. The residue was purified by silica gel chromatograph (DCM:MeOH=20:1) to give the title compound (350 mg, 91%). LC-MS (M+H)⁺=944.7.

### Step 5: (4S)-2-[(2S)-2-{[(tert-butoxy)carbonyl]amino}-3-[4-(2-{5-[(2R,6S)-2,6-dimethylmorpholin-4-yl]-2-[(1S)-1-methoxyethyl]pyridin-3-yl}-3-(3-hydroxy-2,2-dimethylpropyl)-9-oxa-1-azatricyclo[6.3.1.0^{4,12}]dodeca-2,4(12),5,7-tetraen-6-yl)-1,3-thiazol-2-yl]propanoyl]-2,3-diazabicyclo[3.1.1]heptane-4-carboxylic Acid

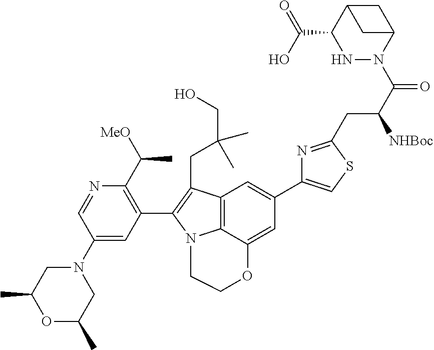 [SMILES: CO[C@@H](C)c1ncc(N2C[C@@H](C)O[C@@H](C)C2)cc1-c1c(CC(C)(C)CO)c2cc(-c3csc(C[C@H](NC(=O)OC(C)(C)C)C(=O)N4N[C@H](C(=O)O)C5CC4C5)n3)cc3c2n1CCO3]

To a mixture of methyl (4S)-2-[(2S)-3-(4-{3-[3-(acetyloxy)-2,2-dimethylpropyl]-2-{5-[(2R,6S)-2,6-dimethylmorpholin-4-yl]-2-[(1S)-1-methoxyethyl]pyridin-3-yl}-9-oxa-1-azatricyclo[6.3.1.0^{4,12}]dodeca-2,4(12),5,7-tetraen-6-yl}-1,3-thiazol-2-yl)-2-{[(tert-butoxy)carbonyl]amino}propanoyl]-2,3-diazabicyclo[3.1.1]heptane-4-carboxylate (350 mg, 0.37 mmol) in THF (5 mL) and water (5 mL) was added LiOH·H₂O (78 mg, 1.85 mmol). The mixture was stirred at room temperature for 2 h. Most of the solvent was removed under vacuum. The pH of the remaining aqueous phase was adjusted to 5 with hydrochloric acid (1 N) at 0° C. The mixture was extracted with DCM (30 mL×2). The combined organic layer was dried over Na₂SO₄, filtered and concentrated under vacuum to give the title compound (330 mg, 100%). LC-MS (M+H)⁺=888.7.

### Step 6: tert-butyl N-[(7S,13S)-20-{5-[(2R,6S)-2,6-dimethylmorpholin-4-yl]-2-[(1S)-1-methoxyethyl]pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate

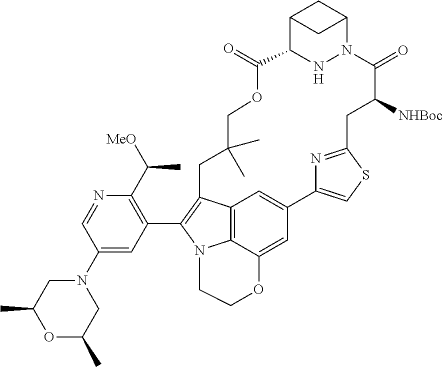 [SMILES: CO[C@@H](C)c1ncc(N2C[C@@H](C)O[C@@H](C)C2)cc1-c1c2c3cc(cc4c3n1CCO4)-c1csc(n1)C[C@H](NC(=O)OC(C)(C)C)C(=O)N1N[C@H](C(=O)OCC(C)(C)C2)C2CC1C2]

To a solution of (4S)-2-[(2S)-2-{[(tert-butoxy)carbonyl]amino}-3-[4-(2-{5-[(2R,6S)-2,6-dimethylmorpholin-4-yl]-2-[(1S)-1-methoxyethyl]pyridin-3-yl}-3-(3-hydroxy-2,2-dimethylpropyl)-9-oxa-1-azatricyclo[6.3.1.04,12]dodeca-2,4(12),5,7-tetraen-6-yl)-1,3-thiazol-2-yl]propanoyl]-2,3-diazabicyclo[3.1.1]heptane-4-carboxylic acid (330 mg, 0.37 mmol) in MeCN (100 mL) was added TCFH (3.22 g, 11.5 mmol) and NMI (943 mg, 11.5 mmol). The mixture was stirred for 2 h at room temperature and concentrated under reduced pressure. The residue was dissolved in EA (100 mL), washed with water (100 mL) and brine (100 mL). The organic layer was concentrated under vacuum. The residue was purified by silica gel chromatograph (DCM:MeOH=20:1) to give the title compound (180 mg, 56%). LC-MS (M+H)⁺=870.7.

### Step 7: (1R,2R,3S)—N-[(7S,13S,19M)-20-{5-[(2R,6S)-2,6-dimethylmorpholin-4-yl]-2-[(1S)-1-methoxyethyl]pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide & (1S,2S)—N-[(7S,13S,19M)-20-{5-[(2R,6S)-2,6-dimethylmorpholin-4-yl]-2-[(1S)-1-methoxyethyl]pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2-methylcyclopropane-1-carboxamide

A mixture of tert-butyl N-[(7S,13S)-20-{5-[(2R,6S)-2,6-dimethylmorpholin-4-yl]-2-[(1S)-1-methoxyethyl]pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate (180 mg, 0.21 mmol) in HCl (4 M n dioxane, 12 mL) was stirred for 4 h at room temperature. The mixture was concentrated under reduced pressure to give a residue (120 mg).

A portion of the residue (60 mg) was re-dissolved in DMF (5 mL) followed by addition of (1r,2R,3S)-2,3-dimethylcyclopropane-1-carboxylic acid (17 mg, 0.15 mmol), HATU (57 mg, 0.15 mmol) and DIPEA (481 mg, 3.7 mmol). The mixture was stirred for 1 h at room temperature. The mixture was diluted with EA (30 mL), washed with brine (10 mL), dried over Na₂SO₄, filtered and concentrated under vacuum. The residue was purified by prep-HPLC to give example 7 (9 mg, 10%). ¹H NMR (400 MHz, DMSO-d6) δ 8.51-8.24 (m, 2H), 8.00 (s, 1H), 7.74 (s, 1H), 7.38-7.03 (m, 2H), 5.87 (d, J=10.9 Hz, 1H), 5.29 (t, J=8.1 Hz, 1H), 4.74-4.41 (m, 4H), 4.37-4.19 (m, 2H), 3.68 (t, J=11.5 Hz, 5H), 3.60-3.44 (m, 2H), 3.25-3.21 (m, 1H), 3.18 (s, 3H), 3.07 (dd, J=14.9, 7.4 Hz, 1H), 2.87 (d, J=14.4 Hz, 1H), 2.65-2.56 (m, 1H), 2.37-2.21 (m, 5H), 2.09 (t, J=9.6 Hz, 1H), 1.55 (t, J=9.3 Hz, 1H), 1.30 (d, J=6.1 Hz, 3H), 1.24-0.94 (m, 15H), 0.88 (s, 3H), 0.33 (s, 3H). LC-MS (M+H)⁺=866.7.

The other portion of the Boc-deprotected residue (60 mg) was re-dissolved in DMF (5 mL) followed by addition of (1S,2S)-2-methylcyclopropane-1-carboxylic acid (15 mg, 0.15 mmol), HATU (57 mg, 0.15 mmol) and DIPEA (481 mg, 3.7 mmol). The mixture was stirred for 1 h at room temperature. The mixture was diluted with EA (30 mL), washed with brine (10 mL), dried over Na₂SO₄, filtered and concentrated under vacuum. The residue was purified by prep-HPLC to give example 8 (7 mg, 8%). ¹H NMR (400 MHz, DMSO-d6) δ 8.50 (d, J=8.9 Hz, 1H), 8.39 (d, J=2.8 Hz, 1H), 8.00 (s, 1H), 7.73 (s, 1H), 7.17 (d, J=2.9 Hz, 1H), 7.12 (s, 1H), 5.88 (d, J=11.0 Hz, 1H), 5.30 (t, J=8.2 Hz, 1H), 4.82-4.39 (m, 4H), 4.42-4.15 (m, 2H), 3.68 (t, J=12.1 Hz, 5H), 3.61-3.45 (m, 2H), 3.26-3.22 (m, 1H), 3.18 (s, 3H), 3.07 (dd, J=14.8, 7.5 Hz, 1H), 2.88 (d, J=14.3 Hz, 1H), 2.67-2.57 (m, 1H), 2.41-2.20 (m, 5H), 2.09 (t, J=9.8 Hz, 1H), 1.56 (t, J=9.3 Hz, 1H), 1.48-1.42 (m, 1H), 1.30 (d, J=6.1 Hz, 3H), 1.17-0.79 (m, 14H), 0.56-0.46 (m, 1H), 0.33 (s, 3H). LC-MS (M+H)⁺=852.7.

Atrop-9 & Example 9: (1R,2R,3S)—N-[(7S,13S,19P)-20-{5-[(9aS)-octahydropyrazino[2,1-c][1,4]oxazin-8-yl]-2-[(1S)-1-methoxyethyl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide & (1R,2R,3S)—N-[(7S,13S,19M)-20-{5-[(9aS)-octahydropyrazino[2,1-c][1,4]oxazin-8-yl]-2-[(1S)-1-methoxyethyl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

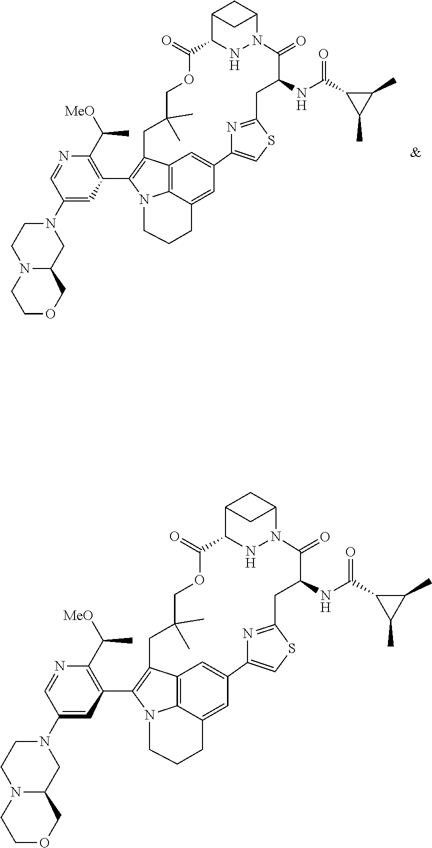

### Step 1: 3-(6-bromo-2-{2-[(1S)-1-methoxyethyl]-5-(4,4,5,5-tetramethyl-1,3,2-dioxaborolan-2-yl)pyridin-3-yl}-1-azatricyclo[6.3.1.0^{4,12}]dodeca-2,4(12),5,7-tetraen-3-yl)-2,2-dimethylpropyl Acetate

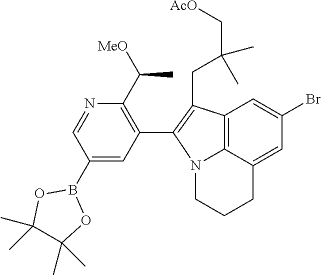 [SMILES: CO[C@@H](C)c1ncc(B2OC(C)(C)C(C)(C)O2)cc1-c1c(CC(C)(C)COC(C)=O)c2cc(Br)cc3c2n1CCC3]

To a mixture of 3-(6-bromo-2-{2-[(1S)-1-methoxyethyl]pyridin-3-yl}-1-azatricyclo[6.3.1.0^{4,12}]dodeca-2,4(12),5,7-tetraen-3-yl)-2,2-dimethylpropyl acetate (2.3 g, 4.6 mmol) and pinacolborane (3.55 g, 27.8 mmol) in THF (35 mL) was added dtbpy (247 mg, 0.92 mmol) and chloro(1,5-cyclooctadiene)iridium(I) dimer (305 mg, 0.46 mmol). The mixture was stirred for 16 h at 80° C. under nitrogen in a sealed tube and then cooled to room temperature. The mixture was poured into iced water (200 mL) with stirring. The mixture was then extracted with EA (200 mL), washed with brine (80 mL), dried over Na₂SO₄, filtered and concentrated under vacuum. The residue was triturated in MeOH (20 mL) and the solid was collected and dried under vacuum to give the title compound (1.7 g, 59%). LC-MS (M+H)⁺=625.3.

### Step 2: 3-(2-{5-[(9aS)-octahydropyrazino[2,1-c][1,4]oxazin-8-yl]-2-[(1S)-1-methoxyethyl]pyridin-3-yl}-6-bromo-1-azatricyclo[6.3.1.0^{4,12}]dodeca-2,4(12),5,7-tetraen-3-yl)-2,2-dimethylpropyl Acetate

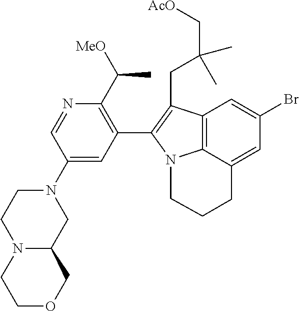 [SMILES: CO[C@@H](C)c1ncc(N2CCN3CCOC[C@@H]3C2)cc1-c1c(CC(C)(C)COC(C)=O)c2cc(Br)cc3c2n1CCC3]

To a mixture of 3-(6-bromo-2-{2-[(1S)-1-methoxyethyl]-5-(4,4,5,5-tetramethyl-1,3,2-dioxaborolan-2-yl)pyridin-3-yl}-1-azatricyclo[6.3.1.0^{4,12}]dodeca-2,4(12),5,7-tetraen-3-yl)-2,2-dimethylpropyl acetate (0.50 g, 0.80 mmol) and (9aS)-octahydropyrazino[2,1-c][1,4]oxazine (802 mg, 8.0 mmol) in MeCN (20 mL) was added Et₃N (243 mg, 2.4 mmol) and Cu(OAc)₂ (291 mg, 1.6 mmol). The mixture was stirred for 4 h at room temperature under oxygen. The mixture was then diluted with EA (60 mL) and water (60 mL). Solid was filtered off and the filter cake was rinsed with EA (30 mL×2). The organic phase of the filtrate was separated and successively washed with water (50 mL) and brine (60 mL), dried over Na₂SO₄, filtered and concentrated under vacuum. The residue was purified by silica gel chromatograph (DCM:MeOH=15:1) to give the title compound (300 mg, 59%). LC-MS (M+H)⁺=639.3.

### Step 3: 3-(2-{5-[(9aS)-octahydropyrazino[2,1-c][1,4]oxazin-8-yl]-2-[(1S)-1-methoxyethyl]pyridin-3-yl}-6-(4,4,5,5-tetramethyl-1,3,2-dioxaborolan-2-yl)-1-azatricyclo[6.3.1.0^{4,12}]dodeca-2,4(12),5,7-tetraen-3-yl)-2,2-dimethylpropyl Acetate

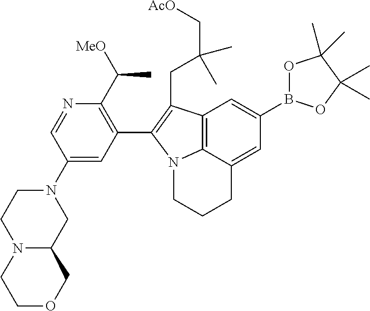 [SMILES: CO[C@@H](C)c1ncc(N2CCN3CCOC[C@@H]3C2)cc1-c1c(CC(C)(C)COC(C)=O)c2cc(B3OC(C)(C)C(C)(C)O3)cc3c2n1CCC3]

To a mixture of 3-(2-{5-[(9aS)-octahydropyrazino[2,1-c][1,4]oxazin-8-yl]-2-[(1S)-1-methoxyethyl]pyridin-3-yl}-6-bromo-1-azatricyclo[6.3.1.0^{4,12}]dodeca-2,4(12),5,7-tetraen-3-yl)-2,2-dimethylpropyl acetate (300 mg, 0.46 mmol) and BPD (308 mg, 1.2 mmol) in toluene (10 mL) was added Pd(dppf)Cl₂·DCM (32 mg, 0.039 mmol) and KOAc (142 mg, 1.45 mmol). The mixture was stirred for 4 h at 90° C. under nitrogen. The mixture was cooled to room temperature and diluted with EA (40 mL). The mixture was washed with brine (20 mL) dried over Na₂SO₄, filtered and concentrated under vacuum. The residue was purified by prep-TLC (DCM:MeOH=15:1) to give the title compound (230 mg, 72%). LC-MS (M+H)⁺=687.5.

### Step 4: methyl (4S)-2-[(2S)-3-[4-(2-{5-[(9aS)-octahydropyrazino[2,1-c][1,4]oxazin-8-yl]-2-[(1S)-1-methoxyethyl]pyridin-3-yl}-3-[3-(acetyloxy)-2,2-dimethylpropyl]-1-azatricyclo[6.3.1.0^{4,12}]dodeca-2,4(12),5,7-tetraen-6-yl)-1,3-thiazol-2-yl]-2-{[(tert-butoxy)carbonyl]amino}propanoyl]-2,3-diazabicyclo[3.1.1]heptane-4-carboxylate

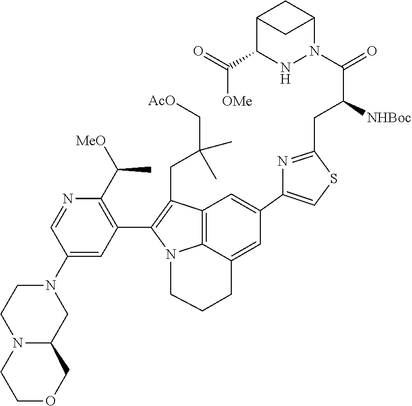 [SMILES: COC(=O)[C@H]1NN(C(=O)[C@H](Cc2nc(-c3cc4c5c(c3)c(CC(C)(C)COC(C)=O)c(-c3cc(N6CCN7CCOC[C@@H]7C6)cnc3[C@H](C)OC)n5CCC4)cs2)NC(=O)OC(C)(C)C)C2CC1C2]

To a mixture of 3-(2-{5-[(9aS)-octahydropyrazino[2,1-c][1,4]oxazin-8-yl]-2-[(1S)-1-methoxyethyl]pyridin-3-yl}-6-(4,4,5,5-tetramethyl-1,3,2-dioxaborolan-2-yl)-1-azatricyclo[6.3.1.0^{4,12}]dodeca-2,4(12),5,7-tetraen-3-yl)-2,2-dimethylpropyl acetate (230 mg, 0.34 mmol) and methyl (4S)-2-[(2S)-3-(4-bromo-1,3-thiazol-2-yl)-2-{[(tert-butoxy)carbonyl]amino}propanoyl]-2,3-diazabicyclo[3.1.1]heptane-4-carboxylate (148 mg, 0.30 mmol) in dioxane (10 mL) and water (2 mL) was added K₃PO₄ (193 mg, 0.912 mmol) and Pd(dtbpf)Cl₂ (20 mg, 0.03 mmol). The mixture was stirred for 3 h at 70° C. under nitrogen and then cooled to room temperature. The mixture was diluted with EA (50 mL) and successively washed with water (20 mL) and brine (20 mL). The organic layer was dried over Na₂SO₄, filtered and concentrated under vacuum. The residue was purified by prep-TLC (DCM:MeOH=10:1) to give the title compound (200 mg, 61%). LC-MS (M+H)⁺=969.5.

### Step 5: (4S)-2-[(2S)-3-[4-(2-{5-[(9aS)-octahydropyrazino[2,1-c][1,4]oxazin-8-yl]-2-[(1S)-1-methoxyethyl]pyridin-3-yl}-3-(3-hydroxy-2,2-dimethylpropyl)-1-azatricyclo[6.3.1.0^{4,12}]dodeca-2,4(12),5,7-tetraen-6-yl)-1,3-thiazol-2-yl]-2-{[(tert-butoxy)carbonyl]amino}propanoyl]-2,3-diazabicyclo[3.1.1]heptane-4-carboxylic Acid

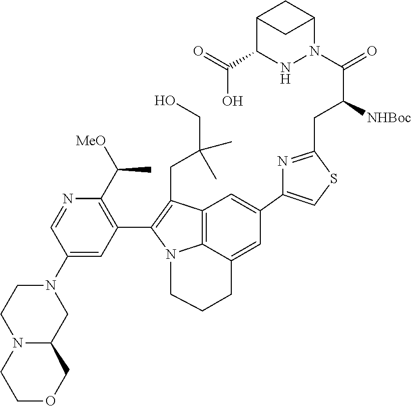 [SMILES: CO[C@@H](C)c1ncc(N2CCN3CCOC[C@@H]3C2)cc1-c1c(CC(C)(C)CO)c2cc(-c3csc(C[C@H](NC(=O)OC(C)(C)C)C(=O)N4N[C@H](C(=O)O)C5CC4C5)n3)cc3c2n1CCC3]

To a mixture of methyl (4S)-2-[(2S)-3-[4-(2-{5-[(9aS)-octahydropyrazino[2,1-c][1,4]oxazin-8-yl]-2-[(1S)-1-methoxyethyl]pyridin-3-yl}-3-[3-(acetyloxy)-2,2-dimethylpropyl]-1-azatricyclo[6.3.1.0^{4,12}]dodeca-2,4(12),5,7-tetraen-6-yl)-1,3-thiazol-2-yl]-2-{[(tert-butoxy)carbonyl]amino}propanoyl]-2,3-diazabicyclo[3.1.1]heptane-4-carboxylate (200 mg, 0.21 mmol) in THF (5 mL) and water (5 mL) was added LiOH·H₂O (84 mg, 2.0 mmol). The mixture was stirred for 6 h at room temperature. The mixture was diluted with water (30 mL) and washed with EA (10 mL). The organic phase was discarded, and the pH of the aqueous phase was adjusted to ˜5 with 1 N hydrochloric acid at 0° C. The mixture was extracted with EA (40 mL×3). The combined organic layer was dried over Na₂SO₄, filtered and concentrated under vacuum to give the title compound (180 mg, 95%). LC-MS (M+H)⁺=913.5.

### Step 6: tert-butyl N-[(7S,13S)-20-{5-[(9aS)-octahydropyrazino[2,1-c][1,4]oxazin-8-yl]-2-[(1S)-1-methoxyethyl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate

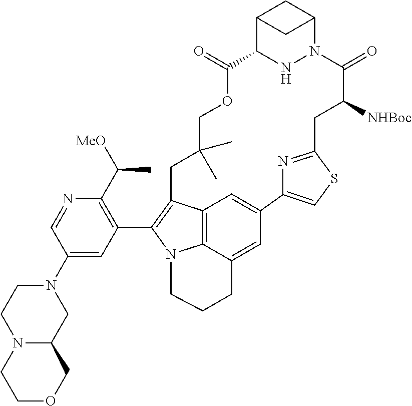 [SMILES: CO[C@@H](C)c1ncc(N2CCN3CCOC[C@@H]3C2)cc1-c1c2c3cc(cc4c3n1CCC4)-c1csc(n1)C[C@H](NC(=O)OC(C)(C)C)C(=O)N1N[C@H](C(=O)OCC(C)(C)C2)C2CC1C2]

To a mixture of (4S)-2-[(2S)-3-[4-(2-{5-[(9aS)-octahydropyrazino[2,1-c][1,4]oxazin-8-yl]-2-[(1S)-1-methoxyethyl]pyridin-3-yl}-3-(3-hydroxy-2,2-dimethylpropyl)-1-azatricyclo[6.3.1.0^{4,12}]dodeca-2,4(12),5,7-tetraen-6-yl)-1,3-thiazol-2-yl]-2-{[(tert-butoxy)carbonyl]amino}propanoyl]-2,3-diazabicyclo[3.1.1]heptane-4-carboxylic acid (180 mg, 0.20 mmol) in MeCN (80 mL) was added TCFH (0.56 g, 2.0 mmol) and NMI (164 mg, 2.0 mmol). The mixture was stirred 2 h at room temperature and then concentrated under reduced pressure. The residue was dissolved in EA (40 mL), washed with 1 N hydrochloric acid (20 mL), water (20 mL) and brine (20 mL). The organic layer was dried over Na₂SO₄, filtered and concentrated under vacuum. The residue was purified by prep-TLC (DCM:MeOH=10:1) to give the title compound (40 mg, 23%). LC-MS (M+H)⁺=895.5.

### Step 7: (7S,13S)-20-{5-[(9aS)-octahydropyrazino[2,1-c][1,4]oxazin-8-yl]-2-[(1S)-1-methoxyethyl]pyridin-3-yl}-7-amino-17,17-dimethyl-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione

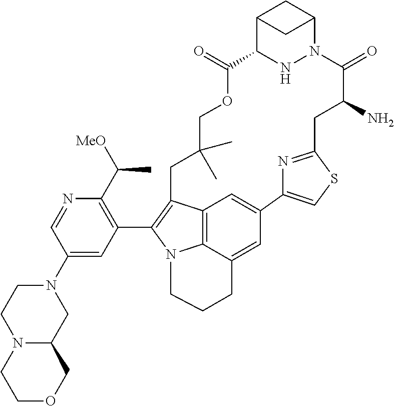 [SMILES: CO[C@@H](C)c1ncc(N2CCN3CCOC[C@@H]3C2)cc1-c1c2c3cc(cc4c3n1CCC4)-c1csc(n1)C[C@H](N)C(=O)N1N[C@H](C(=O)OCC(C)(C)C2)C2CC1C2]

A mixture of tert-butyl N-[(7S,13S)-20-{5-[(9aS)-octahydropyrazino[2,1-c][1,4]oxazin-8-yl]-2-[(1S)-1-methoxyethyl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate (40 mg, 0.04 mmol) in HCG (4 M in dioxane, 3 mL) was stirred for 1 h at room temperature and then concentrated under vacuum. The residue was diluted with DCM (20 mL), washed with saturated NaHCO₃ (10 mL), dried over Na₂SO₄, filtered and concentrated under vacuum to give the title compound (35 mg, 99%/). LC-MS (M+H)⁺=795.5.

### Step 8: (1R,2R,3S)—N-[(7S,13S,19P)-20-{5-[(9aS)-octahydropyrazino[2,1-c][1,4]oxazin-8-yl]-2-[(1S)-1-methoxyethyl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide & (1R,2R,3S)—N-[(7S,13S,19M)-20-{5-[(9aS)-octahydropyrazino[2,1-c][1,4]oxazin-8-yl]-2-[(1S)-1-methoxyethyl]pyridin-3-yl}-17,17-dimethyl-8,14-dioxo-15-exa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

To a mixture of (7S,13S)-20-{5-[(9aS)-octahydropyrazino[2,1-c][1,4]oxazin-8-yl]-2-[(1S)-1-methoxyethyl]pyridin-3-yl}-7-amino-17,17-dimethyl-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione (20 mg, 0.025 mmol) in DMF (4 mL) was added DIPEA (16 mg, 0.125 mmol), (1r,2R,3S)-2,3-dimethylcyclopropane-1-carboxylic acid (5.7 mg, 0.050 mmol) and HATU (19 mg, 0.050 mmol). The mixture was stirred for 2 h at room temperature and diluted with EA (40 mL), washed with brine (10 mL×2). The organic phase was dried over Na₂SO₄, filtered and concentrated under vacuum. The residue was purified by prep-TLC (DCM:MeOH=10:1) followed by prep-HPLC to give atrop-9 (P-isomer, 2.1 mg, 9%) and example 9 (M-isomer, 1.4 mg, 6%).

Atrop-9: ¹H NMR (400 MHz, DMSO-d6) δ 8.43-8.34 (m, 2H), 8.22 (s, 1H), 7.69 (s, 1H), 7.39 (s, 1H), 7.33 (d, J=2.7 Hz, 1H), 5.84 (d, J=11.0 Hz, 1H), 5.27 (t, J=8.0 Hz, 1H), 4.70 (d, J=11.1 Hz, 1H), 4.5-4.4 (m, 1H), 3.96-3.82 (m, 2H), 3.78-3.55 (m, 6H), 3.54-3.41 (m, 3H), 3.17-3.08 (m, 2H), 3.05 (s, 3H), 3.00-2.90 (m, 3H), 2.8-2.72 (m, 2H), 2.66-2.59 (m, 3H), 2.35-2.23 (m, 5H), 2.2-2.10 (m, 3H), 2.01-1.9 (m, 1H), 1.58 (t, J=9.4 Hz, 1H), 1.23-1.14 (m, 2H), 1.12-1.00 (m, 10H), 0.89 (s, 3H), 0.47 (s, 3H). LC-MS (M+H)⁺=891.7.

Example 9: ¹H NMR (400 MHz, DMSO-d6) δ 8.59-8.30 (m, 2H), 8.20 (s, 1H), 7.68 (s, 1H), 7.39 (s, 1H), 7.10 (d, J=2.9 Hz, 1H), 5.89 (d, J=11.0 Hz, 1H), 5.32 (d, J=8.6 Hz, 1H), 4.62 (d, J=11.0 Hz, 1H), 4.59-4.36 (m, 1H), 4.32-4.08 (m, 2H), 3.86-3.42 (m, 10H), 3.23-2.93 (m, 14H), 2.42-2.05 (m, 7H), 1.51 (t, J=9.3 Hz, 1H), 1.29 (d, J=6.0 Hz, 3H), 1.19-0.96 (m, 9H), 0.84 (s, 3H), 0.35 (s, 3H). LC-MS (M+H)⁺=891.7.

Atrop-10 & Example 10: (1S,2S)—N-[(7S,13S,19P)-20-{5-[(9aS)-octahydropyrazino[2,1-c][1,4]oxazin-8-yl]-2-[(1S)-1-methoxyethyl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2-methylcyclopropane-1-carboxamide & (1S,2S)—N-[(7S,13S,19M)-20-{5-[(9aS)-octahydropyrazino[2,1-c][1,4]oxazin-8-yl]-2-[(1S)-1-methoxyethyl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2-methylcyclopropane-1-carboxamide

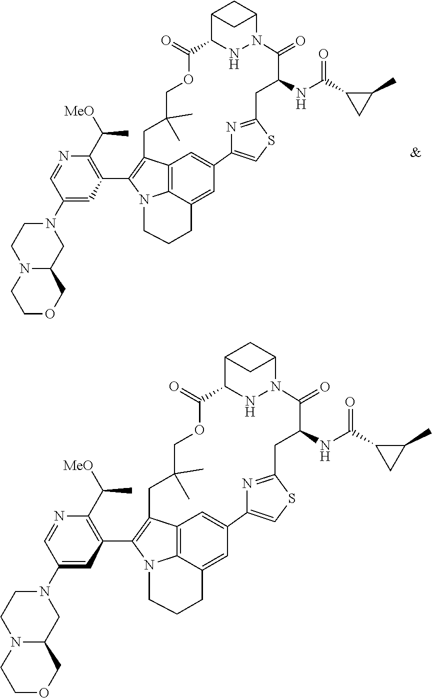

To a mixture of (7S,13S)-20-{5-[(9aS)-octahydropyrazino[2,1-c][1,4]oxazin-8-yl]-2-[(1S)-1-methoxyethyl]pyridin-3-yl}-7-amino-17,17-dimethyl-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione (15 mg, 0.019 mmol) in DMF (4 mL) was added DIPEA (16 mg, 0.125 mmol), (1S,2S)-2-methylcyclopropane-1-carboxylic acid (5 mg, 0.050 mmol) and HATU (19 mg, 0.050 mmol). The mixture was stirred for 2 h at room temperature and then diluted with EA (40 mL), washed with brine (10 mL×2). The organic phase was dried over Na₂SO₄, filtered and concentrated under vacuum. The residue was purified by prep-TLC (DCM:MeOH=10:1) followed by prep-HPLC to give atrop-10 (P-isomer, 1.0 mg, 6%) and example 10 (M-isomer, 1.0 mg, 6%).

Atrop-10: ¹H NMR (400 MHz, DMSO-d6) δ 8.51 (d, J=8.8 Hz, 1H), 8.43 (s, 1H), 8.38 (d, J=2.7 Hz, 1H), 7.68 (s, 1H), 7.39 (s, 1H), 7.33 (d, J=2.8 Hz, 1H), 5.86 (d, J=11.1 Hz, 1H), 5.28 (t, J=8.4 Hz, 1H), 4.70 (d, J=11.2 Hz, 1H), 4.5-4.43 (m, 1H), 3.95-3.80 (m, 3H), 3.76-3.62 (m, 4H), 3.57 (s, 2H), 3.52-3.42 (m, 3H), 3.22-3.16 (m, 2H), 3.14-3.08 (m, 2H), 3.05 (s, 3H), 3.01-2.89 (m, 3H), 2.79-2.72 (m, 3H), 2.65-2.59 (m, 4H), 2.36-2.26 (m, 3H), 2.22-1.91 (m, 4H), 1.58 (t, J=9.5 Hz, 1H), 1.48-1.415 (m, 1H), 1.12-1.05 (m, 3H), 1.03 (s, 3H), 0.93-0.84 (m, 4H), 0.56-0.51 (m, 1H), 0.47 (s, 3H). LC-MS (M+H)⁺=877.7.

Example 10: ¹H NMR (400 MHz, DMSO-d6) δ 8.55-8.34 (m, 2H), 8.19 (s, 1H), 7.67 (s, 1H), 7.39 (s, 1H), 7.10 (d, J=2.9 Hz, 1H), 5.90 (d, J=11.0 Hz, 1H), 5.35 (d, J=8.1 Hz, 1H), 4.63 (d, J=10.9 Hz, 1H), 4.57-4.37 (m, 1H), 4.39-4.02 (m, 2H), 3.84-3.44 (m, 10H), 3.22-2.56 (m, 14H), 2.38-2.05 (m, 7H), 1.60-1.41 (m, 2H), 1.29 (d, J=6.0 Hz, 3H), 1.05-0.79 (m, 8H), 0.58-0.47 (m, 1H), 0.34 (s, 3H). LC-MS (M+H)⁺=877.7.

### Example 11: (1R,2R,3S)—N-[(7S,13S,19M)-20-{2-[(1S)-1-methoxyethyl]-5-(4-methylpiperazin-1-yl)pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

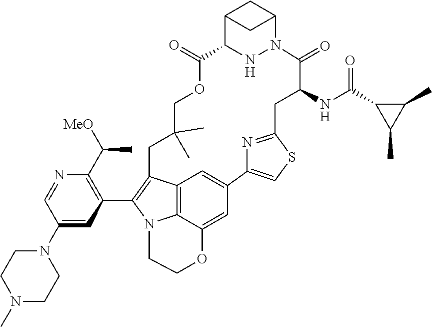

### Step 1: 3-(6-bromo-2-{2-[(1S)-1-methoxyethyl]-5-(4-methylpiperazin-1-yl)pyridin-3-yl}-9-oxa-1-azatricyclo[6.3.1.0^{4,12}]dodeca-2,4(12),5,7-tetraen-3-yl)-2,2-dimethylpropyl Acetate

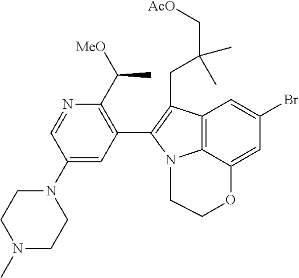

To a mixture of (5-{3-[3-(acetyloxy)-2,2-dimethylpropyl]-6-bromo-9-oxa-1-azatricyclo[6.3.1.0^{4,12}]dodeca-2,4(12),5,7-tetraen-2-yl}-6-[(1S)-1-methoxyethyl]pyridin-3-yl)boronic acid (1.65 g, 3.03 mmol) and 1-methylpiperazine (1.21 g, 12.1 mmol) in MeCN (40 mL) was added Et₃N (918 mg, 9.09 mmol) and Cu(OAc)₂ (1.1 g, 6.06 mmol). The mixture was stirred overnight at room temperature under oxygen and then diluted with EA (150 mL) and water (50 mL). Solid was filtered off and the filter cake was rinsed with EA (50 mL×2). The organic layer of the filtrate was separated and successively washed with water (50 mL), brine (40 mL), dried over Na₂SO₄, filtered and concentrated under vacuum. The residue was purified by silica gel chromatograph (DCM:MeOH=20:1) to give the title compound (1.06 g, 58%). LC-MS (M+H)⁺=599.3/601.3.

### Step 2: 3-(2-{2-[(1S)-1-methoxyethyl]-5-(4-methylpiperazin-1-yl)pyridin-3-yl}-6-(4,4,5,5-tetramethyl-1,3,2-dioxaborolan-2-yl)-9-oxa-1-azatricyclo[6.3.1.0^{4,12}]dodeca-2,4(12),5,7-tetraen-3-yl)-2,2-dimethylpropyl Acetate

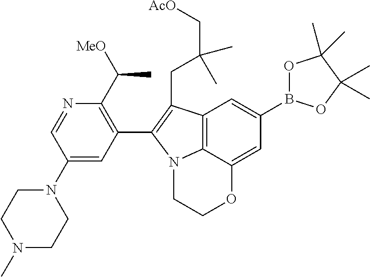 [SMILES: CO[C@@H](C)c1ncc(N2CCN(C)CC2)cc1-c1c(CC(C)(C)COC(C)=O)c2cc(B3OC(C)(C)C(C)(C)O3)cc3c2n1CCO3]

To a mixture of 3-(6-bromo-2-{2-[(1S)-1-methoxyethyl]-5-(4-methylpiperazin-1-yl)pyridin-3-yl}-9-oxa-1-azatricyclo[6.3.1.0^{4,12}]dodeca-2,4(12),5,7-tetraen-3-yl)-2,2-dimethylpropyl acetate (1.01 g, 1.67 mmol) and BPD (847 mg, 3.33 mmol) in toluene (25 mL) was added Pd(dppf)Cl₂·DCM (140 mg, 0.17 mmol) and KOAc (491 mg, 5.0 mmol). The mixture was stirred at 90° C. for 2 h under nitrogen. The mixture was cooled to room temperature and concentrated under reduced pressure. The residue was purified by silica gel chromatograph (DCM:MeOH=10:1) to give the title compound (760 mg, 70%). LC-MS (M+H)⁺=647.4.

### Step 3: methyl (4S)-2-[(2S)-3-(4-{3-[3-(acetyloxy)-2,2-dimethylpropyl]-2-{2-[(1S)-1-methoxyethyl]-5-(4-methylpiperazin-1-yl)pyridin-3-yl}-9-oxa-1-azatricyclo[6.3.1.0^{4,12}]dodeca-2,4(12),5,7-tetraen-6-yl}-1,3-thiazol-2-yl)-2-{[(tert-butoxy)carbonyl]amino}propanoyl]-2,3-diazabicyclo[3.1.1]heptane-4-carboxylate

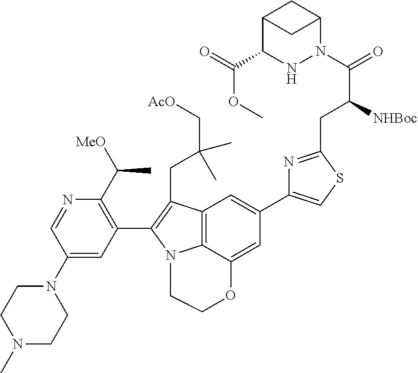 [SMILES: COC(=O)[C@H]1NN(C(=O)[C@H](Cc2nc(-c3cc4c5c(c3)c(CC(C)(C)COC(C)=O)c(-c3cc(N6CCN(C)CC6)cnc3[C@H](C)OC)n5CCO4)cs2)NC(=O)OC(C)(C)C)C2CC1C2]

To a mixture of 3-(2-{2-[(1S)-1-methoxyethyl]-5-(4-methylpiperazin-1-yl)pyridin-3-yl}-6-(4,4,5,5-tetramethyl-1,3,2-dioxaborolan-2-yl)-9-oxa-1-azatricyclo[6.3.1.0^{4,12}]dodeca-2,4(12),5,7-tetraen-3-yl)-2,2-dimethylpropyl acetate (194 mg, 0.30 mmol) and methyl (4S)-2-[(2S)-3-(4-bromo-1,3-thiazol-2-yl)-2-{[(tert-butoxy)carbonyl]amino}propanoyl]-2,3-diazabicyclo[3.1.1]heptane-4-carboxylate (176 mg, 0.36 mmol) in dioxane (5 mL) and water (0.5 mL) was added K₃PO₄ (191 mg, 0.9 mmol) and Pd(dtbpf)Cl₂ (19 mg, 0.031 mmol). The mixture was stirred at 80° C. for 8 h under nitrogen and then cooled to room temperature. The mixture was treated with brine (50 mL) and extracted with EtOAc (50 mL×3). The organic layer was separated, dried over Na₂SO₄, filtered and concentrated under vacuum. The residue was purified by prep-TLC (DCM:MeOH=10:1) to give the title compound (114 mg, 41%). LC-MS (M+H)⁺=929.7.

### Step 4: (4S)-2-[(2S)-2-{[(tert-butoxy)carbonyl]amino}-3-{4-[3-(3-hydroxy-2,2-dimethylpropyl)-2-{2-[(1S)-1-methoxyethyl]-5-(4-methylpiperazin-1-yl)pyridin-3-yl}-9-oxa-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic Acid

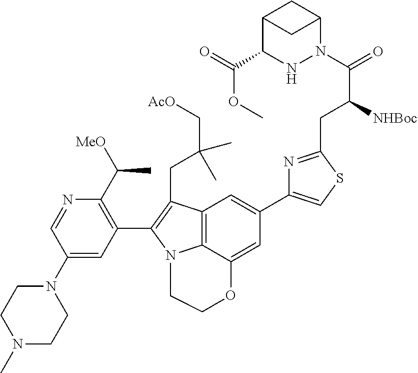 [SMILES: COC(=O)[C@H]1NN(C(=O)[C@H](Cc2nc(-c3cc4c5c(c3)c(CC(C)(C)COC(C)=O)c(-c3cc(N6CCN(C)CC6)cnc3[C@H](C)OC)n5CCO4)cs2)NC(=O)OC(C)(C)C)C2CC1C2]

To a solution of methyl (4S)-2-[(2S)-3-(4-{3-[3-(acetyloxy)-2,2-dimethylpropyl]-2-{2-[(1S)-1-methoxyethyl]-5-(4-methylpiperazin-1-yl)pyridin-3-yl}-9-oxa-1-azatricyclo[6.3.1.0^{4,12}]dodeca-2,4(12),5,7-tetraen-6-yl}-1,3-thiazol-2-yl)-2-{[(tert-butoxy)carbonyl]amino}propanoyl]-2,3-diazabicyclo[3.1.1]heptane-4-carboxylate (114 mg, 0.12 mmol) in THF (2 mL) and water (2 mL) was added LiOH·H₂O (25 mg, 0.60 mmol). The mixture was stirred for 8 h at room temperature. Then mixture was diluted with water (30 mL), and the pH was adjusted to ˜5 with 1 N hydrochloric acid at 0° C. The mixture was extracted with DCM:MeOH (10:1) (30 mL×3). The combined organic layer was dried over Na₂SO₄, filtered and concentrated under vacuum to give the title compound (94 mg, 90%). LC-MS (M+H)⁺=873.6.

### Step 5: tert-butyl N-[(7S,13S)-20-{2-[(S)-1-methoxyethyl]-5-(4-methylpiperazin-1-yl)pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate

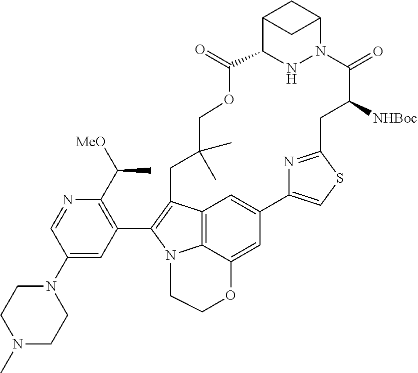 [SMILES: CO[C@@H](C)c1ncc(N2CCN(C)CC2)cc1-c1c2c3cc(cc4c3n1CCO4)-c1csc(n1)C[C@H](NC(=O)OC(C)(C)C)C(=O)N1N[C@H](C(=O)OCC(C)(C)C2)C2CC1C2]

To a solution of (4S)-2-[(2S)-2-{[(tert-butoxy)carbonyl]amino}-3-{4-[3-(3-hydroxy-2,2-dimethylpropyl)-2-{2-[(1S)-1-methoxyethyl]-5-(4-methylpiperazin-1-yl)pyridin-3-yl}-9-oxa-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic acid (94 mg, 0.11 mmol) in anhydrous MeCN (30 mL) was added TCFH (927 mg, 3.3 mmol) and NMI (271 mg, 3.3 mmol). The mixture was stirred for 6 h at room temperature. Then mixture was diluted with water (60 mL), extracted with DCM (50 mL×5). The combined organic layer was washed with brine (30 mL×2), dried over Na₂SO₄, filtered and concentrated under vacuum. The residue was purified by prep-TLC (DCM:MeOH=10:1) to give the title compound (27 mg, 29%). LC-MS (M+H)⁺=855.7.

### Step 6: (1R,2R,3S)—N-[(7S,13S,19M)-20-{2-[(S)-1-methoxyethyl]-5-(4-methylpiperazin-1-yl)pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

To a mixture of tert-butyl N-[(7S,13S)-20-(2-[(1S)-1-methoxyethyl]-5-{4-methylpiperazin-1-yl)pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate (27 mg, 0.03 mmol) in dioxane (1.5 mL) was added HCl (4 M in dioxane, 1.5 mL). The mixture was stirred for 0.5 h at room temperature and concentrated under vacuum. The residue was re-dissolved in DMF (3 mL), followed by addition of (1r,2R,3S)-2,3-dimethylcyclopropane-1-carboxylic acid (6.8 mg, 0.06 mmol), HATU (23 mg, 0.06 mmol) and DIEA (39 mg, 0.31 mmol). The mixture was stirred at room temperature for 1 h. The mixture was diluted with brine (50 mL) and extracted with EtOAc (50 mL×2). The combined organic layer was dried over Na₂SO₄, filtered and concentrated under vacuum. The residue was purified by prep-HPLC to give example 11 (10 mg, 37%). 8.45-8.32 (m, 2H), 8.00 (s, 1H), 7.73 (s, 1H), 7.22-7.03 (m, 2H), 5.86 (d, J=11.0, 1H), 5.29 (t, J=8.2, 1H), 4.71-4.18 (m, 6H), 3.73-3.45 (m, 3H), 3.23-3.13 (m, 8H), 3.11-3.04 (m, 1H), 2.92-2.84 (m, 1H), 2.64-2.57 (m, 1H), 2.43-2.27 (m, 7H), 2.20-2.05 (m, 4H), 1.55 (t, J=9.2, 1H), 1.29 (d, J=8.2, 3H), 1.22-0.95 (m, 9H), 0.88 (s, 3H), 0.32 (s, 3H). LC-MS (M+H)⁺=851.8.

### Example 12: (1S,2S)—N-[(7S,13S,19M)-20-{2-[(1S)-1-methoxyethyl]-5-(4-methylpiperazin-1-yl)pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2-methylcyclopropane-1-carboxamide

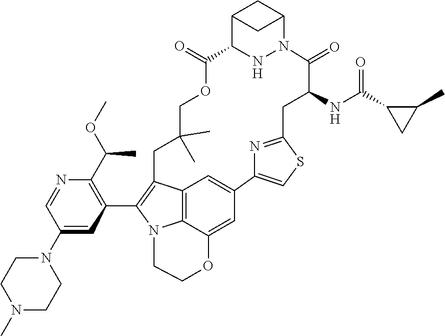

Example 12 (10 mg, 33%) was prepared in a manner similar to that in example 11 step 6 from tert-butyl N-[(7S,13S)-20-{2-[(1S)-1-methoxyethyl]-5-(4-methylpiperazin-1-yl)pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate and (1S,2S)-2-methylcyclopropane-1-carboxylic acid. ¹H NMR (400 MHz, DMSO-d₆) δ 8.50 (d, J=9.0 Hz, 1H), 8.41 (d, J=2.9 Hz, 1H), 8.00 (s, 1H), 7.73 (s, 1H), 7.19 (d, J=2.9 Hz, 1H), 7.12 (s, 1H), 5.88 (d, J=11.0 Hz, 1H), 5.30 (t, J=8.3 Hz, 1H), 4.84-4.42 (m, 4H), 4.38-4.19 (m, 2H), 3.74-3.60 (m, 1H), 3.59-3.45 (m, 2H), 3.28-3.15 (m, 8H), 3.07 (dd, J=15.0, 7.2 Hz, 1H), 2.90 (s, 1H), 2.71-2.26 (m, 11H), 2.09 (t, J=9.8 Hz, 1H), 1.56 (t, J=9.3 Hz, 1H), 1.48-1.41 (m, 1H), 1.29 (d, J=6.1 Hz, 3H), 1.09-0.82 (m, 8H), 0.59-0.47 (m, 1H), 0.32 (s, 3H). LC-MS (M+H)⁺=837.4.

### Example 14: (1R,2R,3S)—N-[(7S,13S,19M)-20-{2-[(1S)-1-methoxyethyl]-5-[(3R,5S)-3,4,5-trimethylpiperazin-1-yl]pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

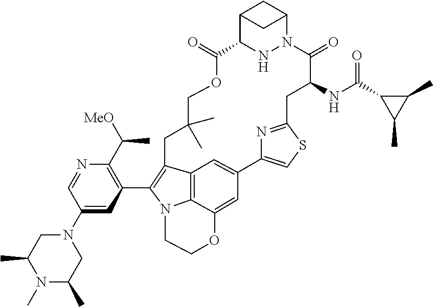

### Step 1: 3-(6-bromo-2-{2-[(1S)-1-methoxyethyl]-5-[(3R,5S)-3,4,5-trimethylpiperazin-1-yl]pyridin-3-yl}-9-oxa-1-azatricyclo[6.3.1.0^{4,12}]dodeca-2,4(12),5,7-tetraen-3-yl)-2,2-dimethylpropyl Acetate

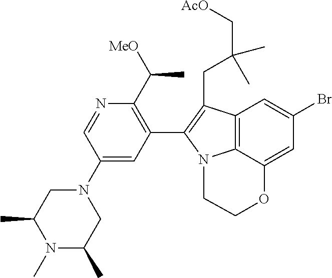 [SMILES: CO[C@@H](C)c1ncc(N2C[C@@H](C)N(C)[C@@H](C)C2)cc1-c1c(CC(C)(C)COC(C)=O)c2cc(Br)cc3c2n1CCO3]

To a solution of (5-{3-[3-(acetyloxy)-2,2-dimethylpropyl]-6-bromo-9-oxa-1-azatricyclo[6.3.1.0^{4,12}]dodeca-2,4(12),5,7-tetraen-2-yl}-6-[(1S)-1-methoxyethyl]pyridin-3-yl)boronic acid (1.09 g, 2.00 mmol) in MeCN (20 mL) was added (2R,6S)-1,2,6-trimethylpiperazine (1.02 g, 8.00 mmol), Cu(OAc)₂ (728 mg, 4.00 mmol) and Et₃N (606 mg, 6.00 mmol). The mixture was stirred at room temperature overnight under oxygen. The mixture was diluted with brine (50 mL) and DCM (50 mL). The solids was filtered off, and the filter cake was rinsed with DCM:MeOH (10:1, 300 mL). The organic layer of the filtrate was separated and dried over Na₂SO₄, filtered and concentrated under vacuum. The residue was purified by silica gel chromatograph (DCM:MeOH=10:1) to give the title compound (610 mg, 49%). LC-MS (M+H)⁺=627.5/629.5.

### Step 2: 3-(2-{2-[(1S)-1-methoxyethyl]-5-[(3R,5S)-3,4,5-trimethylpiperazin-1-yl]pyridin-3-yl}-6-(4,4,5,5-tetramethyl-1,3,2-dioxaborolan-2-yl)-9-oxa-1-azatricyclo[6.3.1.0^{4,12}]dodeca-2,4(12),5,7-tetraen-3-yl)-2,2-dimethylpropyl Acetate

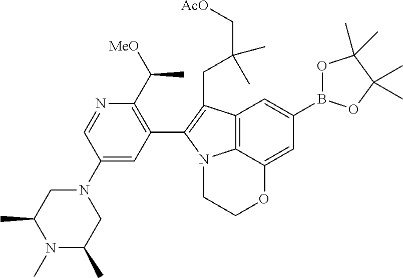

To a mixture of 3-(6-bromo-2-{2-[(1S)-1-methoxyethyl]-5-[(3R,5S)-3,4,5-trimethylpiperazin-1-yl]pyridin-3-yl}-9-oxa-1-azatricyclo[6.3.1.0^{4,12}]dodeca-2,4(12),5,7-tetraen-3-yl)-2,2-dimethylpropyl acetate (610 mg, 0.97 mmol) and BPD (739 mg, 2.91 mmol) in dioxane (10 mL) was added Pd(dppf)Cl₂·DCM (79 mg, 0.097 mmol) and KOAc (327 mg, 3.34 mmol). The mixture was stirred at 80° C. for 6 h under nitrogen. The mixture was cooled to room temperature, diluted with EA (50 mL) and then washed with brine (40 mL). The organic layer was dried over Na₂SO₄, filtered and concentrated under vacuum. The residue was purified by silica gel chromatograph (DCM:MeOH=10:1) to give the title compound (608 mg, 92%). LC-MS (M+H)⁺=675.6.

### Step 3: methyl (4S)-2-[(2S)-3-(4-{3-[3-(acetyloxy)-2,2-dimethylpropyl]-2-{2-[(1S)-1-methoxyethyl]-5-[(3R,5S)-3,4,5-trimethylpiperazin-1-yl]pyridin-3-yl}-9-oxa-1-azatricyclo[6.3.1.0^{4,12}]dodeca-2,4(12),5,7-tetraen-6-yl}-1,3-thiazol-2-yl)-2-{[(tert-butoxy)carbonyl]amino}propanoyl]-2,3-diazabicyclo[3.1.1]heptane-4-carboxylate

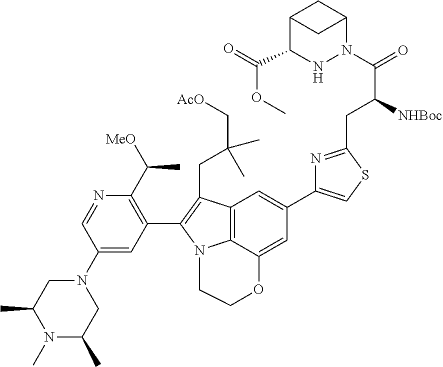 [SMILES: COC(=O)[C@H]1NN(C(=O)[C@H](Cc2nc(-c3cc4c5c(c3)c(CC(C)(C)COC(C)=O)c(-c3cc(N6C[C@@H](C)N(C)[C@@H](C)C6)cnc3[C@H](C)OC)n5CCO4)cs2)NC(=O)OC(C)(C)C)C2CC1C2]

To a mixture of 3-(2-{2-[(1S)-1-methoxyethyl]-5-[(3R,5S)-3,4,5-trimethylpiperazin-1-yl]pyridin-3-yl}-6-(4,4,5,5-tetramethyl-1,3,2-dioxaborolan-2-yl)-9-oxa-1-azatricyclo[6.3.1.0^{4,12}]dodeca-2,4(12),5,7-tetraen-3-yl)-2,2-dimethylpropyl acetate (270 mg, 0.40 mmol) and methyl (4S)-2-[(2S)-3-(4-bromo-1,3-thiazol-2-yl)-2-{[(tert-butoxy)carbonyl]amino}propanoyl]-2,3-diazabicyclo[3.1.1]heptane-4-carboxylate (234 mg, 0.48 mmol) in dioxane (5 mL) and water (0.5 mL) was added K₃PO₄ (253 mg, 1.20 mmol) and Pd(dtbpf)Cl₂ (26 mg, 0.04 mmol). The mixture was stirred for 8 h at 90° C. under nitrogen and cooled to room temperature. The mixture was diluted with brine (50 mL) and extracted with EtOAc (50 mL×3). The combined organic layer was dried over Na₂SO₄, filtered and concentrated under vacuum. The residue was purified by prep-TLC (DCM:MeOH=10:1) to give the title compound (160 mg, 42%). LC-MS (M+H)⁺=957.7.

### Step 4: (4S)-2-[(2S)-2-{[(tert-butoxy)carbonyl]amino}-3-{4-[3-(3-hydroxy-2,2-dimethylpropyl)-2-{2-[(1S)-1-methoxyethyl]-5-[(3R,5S)-3,4,5-trimethylpiperazin-1-yl]pyridin-3-yl}-9-oxa-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic Acid

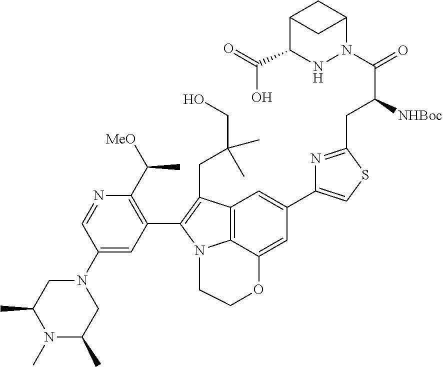 [SMILES: CO[C@@H](C)c1ncc(N2C[C@@H](C)N(C)[C@@H](C)C2)cc1-c1c(CC(C)(C)CO)c2cc(-c3csc(C[C@H](NC(=O)OC(C)(C)C)C(=O)N4N[C@H](C(=O)O)C5CC4C5)n3)cc3c2n1CCO3]

To a solution of methyl (4S)-2-[(2S)-3-(4-{3-[3-(acetyloxy)-2,2-dimethylpropyl]-2-{2-[(1S)-1-methoxyethyl]-5-[(3R,5S)-3,4,5-trimethylpiperazin-1-yl]pyridin-3-yl}-9-oxa-1-azatricyclo[6.3.1.0^{4,12}]dodeca-2,4(12),5,7-tetraen-6-yl}-1,3-thiazol-2-yl)-2-{[(tert-butoxy)carbonyl]amino}propanoyl]-2,3-diazabicyclo[3.1.1]heptane-4-carboxylate (160 mg, 0.17 mmol) in THF (4 mL) and water (4 mL) was added LiOH·H₂O (36 mg, 0.85 mmol). The mixture was stirred for 10 h at room temperature and diluted with water (30 mL). The pH of the mixture was adjusted to ˜5 with 1 N hydrochloric acid at 0° C. The mixture was extracted with DCM:MeOH (10:1, 30 mL×7). The combined organic layer was dried over Na₂SO₄, filtered and concentrated under vacuum to give the title compound (130 mg, 85%). LC-MS (M+H)⁺=901.7.

### Step 5: tert-butyl N-[(7S,13S)-20-{2-[(1S)-1-methoxyethyl]-5-[(3R,5S)-3,4,5-trimethylpiperazin-1-yl]pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate

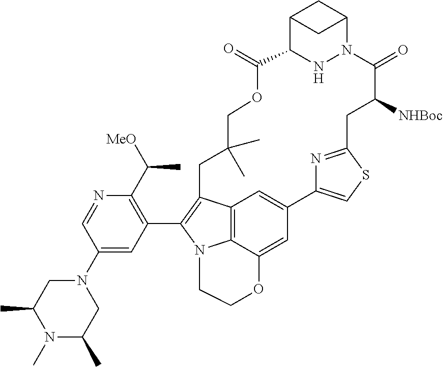 [SMILES: CO[C@@H](C)c1ncc(N2C[C@@H](C)N(C)[C@@H](C)C2)cc1-c1c2c3cc(cc4c3n1CCO4)-c1csc(n1)C[C@H](NC(=O)OC(C)(C)C)C(=O)N1N[C@H](C(=O)OCC(C)(C)C2)C2CC1C2]

To a mixture of (4S)-2-[(2S)-2-{[(tert-butoxy)carbonyl]amino}-3-{4-[3-(3-hydroxy-2,2-dimethylpropyl)-2-{2-[(1S)-1-methoxyethyl]-5-[(3R,5S)-3,4,5-trimethylpiperazin-1-yl]pyridin-3-yl}-9-oxa-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic acid (130 mg, 0.14 mmol) in anhydrous MeCN (50 mL) was added TCFH (1.18 mg, 4.20 mmol) and NMI (344 mg, 4.20 mmol). The mixture was stirred at room temperature for 10 h and diluted with water (60 mL). The mixture was extracted with DCM (50 mL×4). The combined organic layer was washed with brine (30 mL×3), dried over Na₂SO₄, filtered and concentrated under vacuum. The residue was purified by prep-TLC (DCM:MeOH=10:1) to give the title compound (13 mg, 10%). LC-MS (M+H)⁺=883.7.

### Step 6: (1R,2R,3S)—N-[(7S,13S,19M)-20-{2-[(1S)-1-methoxyethyl]-5-[(3R,5S)-3,4,5-trimethylpiperazin-1-yl]pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

To a mixture of tert-butyl N-[(7S,13S)-20-{2-[(1S)-1-methoxyethyl]-5-[(3R,5S)-3,4,5-trimethylpiperazin-1-yl]pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate (13 mg, 0.015 mmol) in dioxane (1.5 mL) was added HCl (4 M in dioxane, 1.5 mL). The mixture was stirred for 0.5 h and concentrated under vacuum. The residue was re-dissolved in DMF (3 mL), followed by addition of (1r,2R,3S)-2,3-dimethylcyclopropane-1-carboxylic acid (3.4 mg, 0.030 mmol), HATU (11.4 mg, 0.030 mmol) and DIEA (19.3 mg, 0.15 mmol). The mixture was stirred at room temperature for 1 h. The mixture was diluted with brine (20 mL) and extracted with EtOAc (30 mL×2). The combined organic layer was dried over Na₂SO₄, filtered and concentrated under vacuum. The residue was purified by prep-HPLC to give example 14 (5 mg, 35%). δ 8.44 (d, J=2.9 Hz, 1H), 8.41 (d, J=8.9 Hz, 1H), 8.06 (s, 1H), 7.77 (s, 1H), 7.20 (d, J=2.9 Hz, 1H), 7.16 (d, J=1.1 Hz, 1H), 5.90 (d, J=11.0 Hz, 1H), 5.35 (t, J=8.0 Hz, 1H), 4.71 (d, J=11.0 Hz, 1H), 4.64-4.54 (m, 2H), 4.50 (q, J=4.9 Hz, 1H), 4.36-4.26 (m, 2H), 3.78-3.66 (m, 3H), 3.61 (d, J=10.8 Hz, 1H), 3.55 (d, J=10.7 Hz, 1H), 3.28-3.19 (m, 4H), 3.13 (dd, J=14.8, 7.4 Hz, 1H), 2.93 (d, J=14.3 Hz, 1H), 2.69-2.62 (m, 1H), 2.48-2.45 (m, 4H), 2.36-2.09 (m, 7H), 1.58 (t, J=9.4 Hz, 1H), 1.35 (d, J=6.1 Hz, 3H), 1.28-1.15 (m, 3H), 1.14-1.01 (m, 12H), 0.93 (s, 3H), 0.38 (s, 3H). LC-MS (M+H)⁺=879.4.

Atrop-15 and Example 15: (1R,2R,3S)—N-[(7S,13S,19P)-20-{2-[(1S)-1-methoxyethyl]-5-{4-oxa-7-azaspiro[2.5]octan-7-yl}pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide & (1R,2R,3S)—N-[(7S,13S,19M)-20-{2-[(1S)-1-methoxyethyl]-5-{4-oxa-7-azaspiro[2.5]octan-7-yl}pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

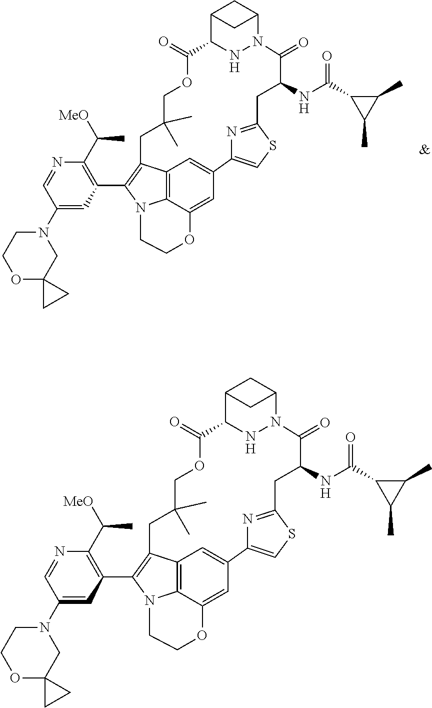

### Step 1: 3-(6-bromo-2-{5-iodo-2-[(1S)-1-methoxyethyl]pyridin-3-yl}-9-oxa-1-azatricyclo[6.3.1.0^{4,12}]dodeca-2,4(12),5,7-tetraen-3-yl)-2,2-dimethylpropyl Acetate

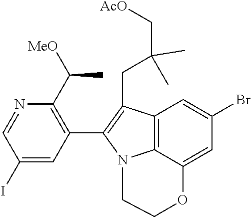 [SMILES: CO[C@@H](C)c1ncc(I)cc1-c1c(CC(C)(C)COC(C)=O)c2cc(Br)cc3c2n1CCO3]

To a solution of (5-{3-[3-(acetyloxy)-2,2-dimethylpropyl]-6-bromo-9-oxa-1-azatricyclo[6.3.1.0^{4,12}]dodeca-2,4(12),5,7-tetraen-2-yl}-6-[(1S)-1-methoxyethyl]pyridin-3-yl)boronic acid (2.7 g, 5.0 mol) and chloramine-T (N-chloro-p-toluenesulfonylamide) (6.8 g, 30 mmol) in THF (30 mL) was added a solution of NaI (4.5 g, 30 mmol) in water (8 mL) dropwise at 0° C. under nitrogen. The mixture was stirred at 50° C. for 3 days and then cooled to room temperature. The mixture was diluted with water (100 mL) and extracted by EtOAc (50 mL×3). The combined organic layer was dried over Na₂SO₄, filtered and concentrated under vacuum. The residue was purified by silica gel chromatograph (PE:EA=3:1) to give the title compound (1.2 g, 38%). LC-MS (M+H)⁺=627.3/629.3.

### Step 2: 3-(6-bromo-2-{2-[(1S)-1-methoxyethyl]-5-{4-oxa-7-azaspiro[2.5]octan-7-yl}pyridin-3-yl}-9-oxa-1-azatricyclo[6.3.1.0^{4,12}]dodeca-2,4(12),5,7-tetraen-3-yl)-2,2-dimethylpropyl Acetate

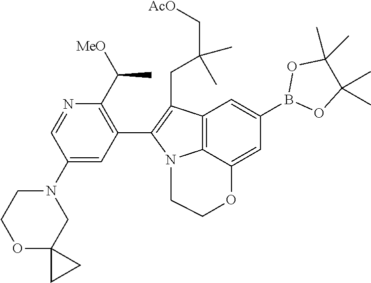

A mixture of 3-(6-bromo-2-{5-iodo-2-[(1S)-1-methoxyethyl]pyridin-3-yl}-9-oxa-1-azatricyclo[6.3.1.0^{4,12}]dodeca-2,4(12),5,7-tetraen-3-yl)-2,2-dimethylpropyl acetate (626 mg, 1.0 mmol), 4-oxa-7-azaspiro[2.5]octane hydrochloride (210 mg, 1.4 mmol), Pd(OAc)₂ (22 mg, 0.10 mmol), Cs₂CO₃ (1.14 g, 3.50 mmol) in toluene (5 mL) was stirred at 90° C. for 12 h and cooled to room temperature. The mixture was diluted with water (100 mL) and extracted with EtOAc (50 mL×2). The combined organic layer was dried over Na₂SO₄, filtered and concentrated under vacuum. The residue was purified by silica gel chromatograph (PE:EA=1:1) to give the title compound (345 mg, 56%). LC-MS (M+H)⁺=612.3/614.3.

### Step 3: 3-(2-{2-[(1S)-1-methoxyethyl]-5-{4-oxa-7-azaspiro[2.5]octan-7-yl}pyridin-3-yl}-6-(4,4,5,5-tetramethyl-1,3,2-dioxaborolan-2-yl)-9-oxa-1-azatricyclo[6.3.1.0^{4,12}]dodeca-2,4(12),5,7-tetraen-3-yl)-2,2-dimethylpropyl Acetate

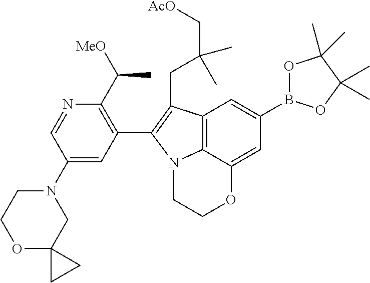 [SMILES: CO[C@@H](C)c1ncc(N2CCOC3(CC3)C2)cc1-c1c(CC(C)(C)COC(C)=O)c2cc(B3OC(C)(C)C(C)(C)O3)cc3c2n1CCO3]

To a mixture of 3-(6-bromo-2-{2-[(1S)-1-methoxyethyl]-5-{4-oxa-7-azaspiro[2.5]octan-7-yl}pyridin-3-yl}-9-oxa-1-azatricyclo[6.3.1.0^{4,12}]dodeca-2,4(12),5,7-tetraen-3-yl)-2,2-dimethylpropyl acetate (245 mg, 0.40 mmol) and BPD (305 mg, 1.20 mmol) in dioxane (10 mL) was added Pd(dppf)Cl₂·DCM (33 mg, 0.04 mmol) and KOAc (137 mg, 1.40 mmol). The mixture was stirred at 100° C. for 12 h under nitrogen. The mixture was cooled to room temperature, diluted with EA (100 mL) and washed with brine (40 mL). The organic phase was dried over Na₂SO₄, filtered and concentrated under vacuum. The crude product was purified by silica gel chromatograph (DCM:MeOH=10:1) to give the title compound (236 mg, 89%). LC-MS (M+H)⁺=660.5.

### Step 4: methyl (4S)-2-[(2S)-3-(4-{3-[3-(acetyloxy)-2,2-dimethylpropyl]-2-{2-[(1S)-1-methoxyethyl]-5-{4-oxa-7-azaspiro[2.5]octan-7-yl}pyridin-3-yl}-9-oxa-1-azatricyclo[6.3.1.0^{4,12}]dodeca-2,4(12),5,7-tetraen-6-yl}-1,3-thiazol-2-yl)-2-{[(tert-butoxy)carbonyl]amino}propanoyl]-2,3-diazabicyclo[3.1.1]heptane-4-carboxylate

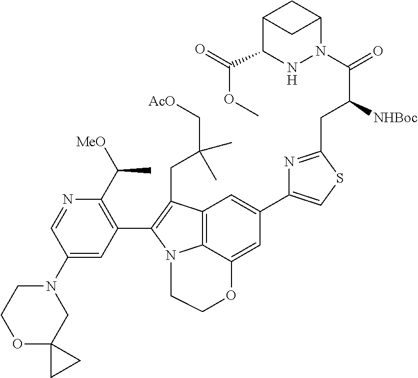 [SMILES: COC(=O)[C@H]1NN(C(=O)[C@H](Cc2nc(-c3cc4c5c(c3)c(CC(C)(C)COC(C)=O)c(-c3cc(N6CCOC7(CC7)C6)cnc3[C@H](C)OC)n5CCO4)cs2)NC(=O)OC(C)(C)C)C2CC1C2]

To a mixture of 3-(2-{2-[(1S)-1-methoxyethyl]-5-{4-oxa-7-azaspiro[2.5]octan-7-yl}pyridin-3-yl}-6-(4,4,5,5-tetramethyl-1,3,2-dioxaborolan-2-yl)-9-oxa-1-azatricyclo[6.3.1.0^{4,12}]dodeca-2,4(12),5,7-tetraen-3-yl)-2,2-dimethylpropyl acetate (236 mg, 0.36 mmol) and methyl (4S)-2-[(2S)-3-(4-bromo-1,3-thiazol-2-yl)-2-{[(tert-butoxy)carbonyl]amino}propanoyl]-2,3-diazabicyclo[3.1.1]heptane-4-carboxylate (210 mg, 0.43 mmol) in dioxane (5 mL) and water (0.5 mL) was added K₃PO₄ (229 mg, 1.08 mmol) and Pd(dtbpf)Cl₂ (23 mg, 0.036 mmol). The mixture was stirred at 80° C. for 8 h under nitrogen and cooled to room temperature. The mixture was diluted with brine (50 mL) and extracted with EtOAc (50 mL×3). The combined organic layer was dried over Na₂SO₄, filtered and concentrated under vacuum. The residue was purified by prep-TLC (DCM:MeOH=10:1) to give the title compound (132 mg, 39%). LC-MS (M+H)⁺=942.6.

### Step 5: (4S)-2-[(2S)-2-{[(tert-butoxy)carbonyl]amino}-3-{4-[3-(3-hydroxy-2,2-dimethylpropyl)-2-{2-[(1S)-1-methoxyethyl]-5-{4-oxa-7-azaspiro[2.5]octan-7-yl}pyridin-3-yl}-9-oxa-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic Acid

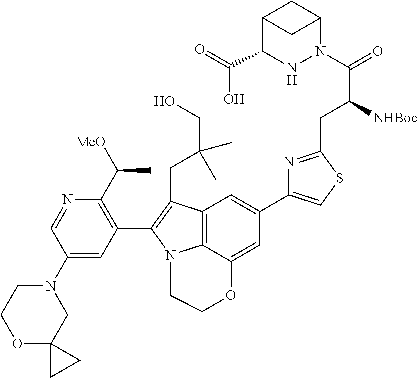 [SMILES: CO[C@@H](C)c1ncc(N2CCOC3(CC3)C2)cc1-c1c(CC(C)(C)CO)c2cc(-c3csc(C[C@H](NC(=O)OC(C)(C)C)C(=O)N4N[C@H](C(=O)O)C5CC4C5)n3)cc3c2n1CCO3]

To a solution of methyl (4S)-2-[(2S)-3-(4-{3-[3-(acetyloxy)-2,2-dimethylpropyl]-2-{2-[(1S)-1-methoxyethyl]-5-{4-oxa-7-azaspiro[2.5]octan-7-yl}pyridin-3-yl}-9-oxa-1-azatricyclo[6.3.1.0^{4,12}]dodeca-2,4(12),5,7-tetraen-6-yl}-1,3-thiazol-2-yl)-2-{[(tert-butoxy)carbonyl]amino}propanoyl]-2,3-diazabicyclo[3.1.1]heptane-4-carboxylate (132 mg, 0.14 mmol) in THF (2 mL) and water (2 mL) was added LiOH·H₂O (30 mg, 0.70 mmol). The mixture was stirred at room temperature for 10 h and diluted with water (30 mL). The pH of the mixture was adjusted to ˜5 with 1 N hydrochloric acid at 0° C. The mixture was extracted with DCM:MeOH (10:1) (30 mL×5). The combined organic layer was dried over Na₂SO₄, filtered and concentrated under vacuum to give the title compound (95 mg, 77%). LC-MS (M+H)⁺=886.6.

### Step 6: tert-butyl N-[(7S,13S)-20-{2-[(1S)-1-methoxyethyl]-5-{4-oxa-7-azaspiro[2.5]octan-7-yl}pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate

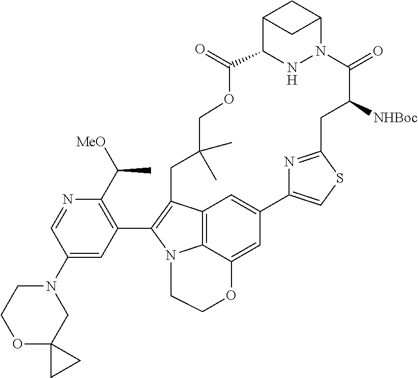 [SMILES: CO[C@@H](C)c1ncc(N2CCOC3(CC3)C2)cc1-c1c2c3cc(cc4c3n1CCO4)-c1csc(n1)C[C@H](NC(=O)OC(C)(C)C)C(=O)N1N[C@H](C(=O)OCC(C)(C)C2)C2CC1C2]

To a solution of (4S)-2-[(2S)-2-{[(tert-butoxy)carbonyl]amino}-3-{4-[3-(3-hydroxy-2,2-dimethylpropyl)-2-{2-[(1S)-1-methoxyethyl]-5-{4-oxa-7-azaspiro[2.5]octan-7-yl}pyridin-3-yl}-9-oxa-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic acid (75 mg, 0.085 mmol) in anhydrous acetonitrile (30 mL) was added TCFH (594 mg, 2.12 mmol) and NMI (174 mg, 2.12 mmol). The mixture was stirred at room temperature for 10 h. Then mixture was diluted with water (60 mL) and extracted with DCM (50 mL×4). The combined organic layer was washed with brine (30 mL×2) dried over Na₂SO₄, filtered and concentrated under. The residue was purified by prep-TLC (DCM:MeOH=10:1) to give the title compound (30 mg, 41%). LC-MS (M+H)⁺=868.6.

### Step 7: (1R,2R,3S)—N-[(7S,13S,19P)-20-{2-[(1S)-1-methoxyethyl]-5-{4-oxa-7-azaspiro[2.5]octan-7-yl}pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide & (1R,2R,3S)—N-[(7S,13S,19M)-20-{2-[(1S)-1-methoxyethyl]-5-{4-oxa-7-azaspiro[2.5]octan-7-yl}pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

mixture of tert-butyl N-[(7S,13S)-20-{2-[(1S)-1-methoxyethyl]-5-{4-oxa-7-azaspiro[2.5]octan-7-yl}pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate (30 mg, 0.034 mmol) in dioxane (1.5 mL) was added HCl (4 M in dioxane, 1.5 mL). The mixture was stirred for 0.5 h and concentrated under vacuum. The residue was re-dissolved in DMF (3 mL) followed by addition of (1r,2R,3S)-2,3-dimethylcyclopropane-1-carboxylic acid (7.7 mg, 0.068 mmol), HATU (25.8 mg, 0.068 mmol) and DIPEA (43.7 mg, 0.34 mmol). The mixture was stirred at room temperature for 1 h, diluted with brine (50 mL) and extracted with EtOAc (50 mL×2). The combined organic layer was dried over Na₂SO₄, filtered and concentrated under vacuum. The residue was purified by prep-HPLC to give atrop-15 (P-isomer, 3.0 mg, 10%) and example 15 (M-isomer, 7.2 mg, 24%).

Atrop-15: ¹H NMR (400 MHz, DMSO-d6) δ 8.45-8.31 (m, 2H), 8.03 (s, 1H), 7.73 (s, 1H), 7.37 (d, J=2.6 Hz, 1H), 7.12 (s, 1H), 5.83 (d, J=10.8 Hz, 1H), 5.26 (t, J=8.2 Hz, 1H), 4.69 (d, J=11.0 Hz, 1H), 4.51-4.34 (m, 3H), 4.06-3.95 (m, 2H), 3.79-3.44 (m, 5H), 3.22-3.03 (m, 8H), 2.96 (d, J=14.2 Hz, 1H), 2.42-2.29 (m, 4H), 2.09 (t, J=9.8 Hz, 1H), 1.58 (t, J=9.6 Hz, 1H), 1.24-1.16 (m, 2H), 1.13-0.97 (m, 11H), 0.90 (s, 3H), 0.70-0.51 (m, 4H), 0.45 (s, 3H). LC-MS (M+H)⁺=864.7

Example 15: ¹H NMR (400 MHz, DMSO-d6) δ 8.59-8.26 (m, 2H), 8.00 (s, 1H), 7.73 (s, 1H), 7.39-6.66 (m, 2H), 5.87 (d, J=11.0 Hz, 1H), 5.29 (t, J=8.2 Hz, 1H), 4.98-4.38 (m, 4H), 4.44-4.10 (m, 2H), 4.02-3.41 (m, 5H), 3.25-3.15 (m, 6H), 3.07 (dd, J=14.8, 7.4 Hz, 1H), 2.87 (d, J=14.3 Hz, 1H), 2.65-2.47 (m, 4H), 2.37-2.24 (m, 2H), 2.09 (t, J=9.8 Hz, 1H), 1.55 (t, J=9.3 Hz, 1H), 1.30 (d, J=6.1 Hz, 3H), 1.23-0.93 (m, 9H), 0.89 (s, 3H), 0.76-0.65 (m, 2H), 0.64-0.51 (m, 2H), 0.33 (s, 3H). LC-MS (M+H)⁺=864.7.

Atrop-16 & Example 16: ((1R,2R,3S)—N-[(7S,13S,19P)-20-{5-[(2R,6S)-2,6-dimethylmorpholin-4-yl]-2-[(1S)-1-methoxyethyl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide & (1R,2R,3S)—N-[(7S,13S,19M)-20-{5-[(2R,6S)-2,6-dimethylmorpholin-4-yl]-2-[(1S)-1-methoxyethyl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

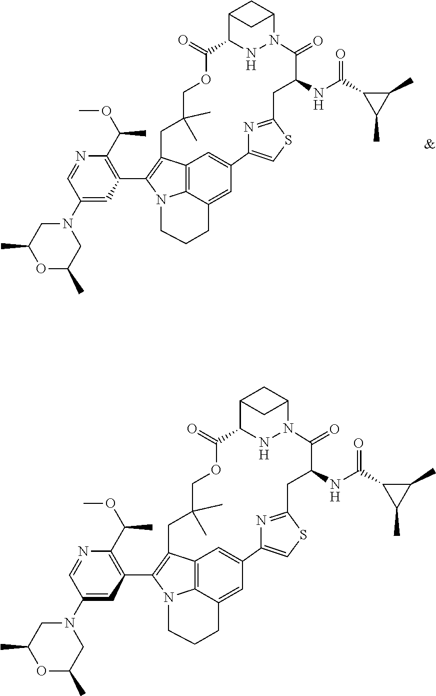

### Step 1: 3-(6-bromo-2-{5-[(2R,6S)-2,6-dimethylmorpholin-4-yl]-2-[(1S)-1-methoxyethyl]pyridin-3-yl}-1-azatricyclo[6.3.1.0^{4,12}]dodeca-2,4(12),5,7-tetraen-3-yl)-2,2-dimethylpropyl Acetate

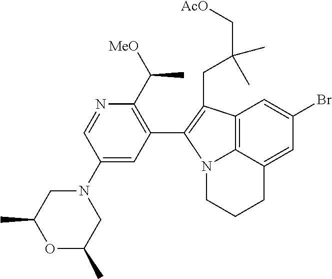 [SMILES: CO[C@@H](C)c1ncc(N2C[C@@H](C)O[C@@H](C)C2)cc1-c1c(CC(C)(C)COC(C)=O)c2cc(Br)cc3c2n1CCC3]

The title compound (330 mg, 61%) was prepared in a manner similar to that in example 9 step 2 from 3-(6-bromo-2-{2-[(1S)-1-methoxyethyl]-5-(4,4,5,5-tetramethyl-1,3,2-dioxaborolan-2-yl)pyridin-3-yl}-1-azatricyclo[6.3.1.0^{4,12}]dodeca-2,4(12),5,7-tetraen-3-yl)-2,2-dimethylpropyl acetate and (2R,6S)-2,6-dimethylmorpholine. LC-MS (M+H)⁺=612.3.

### Step 2: 3-(2-{5-[(2R,6S)-2,6-dimethylmorpholin-4-yl]-2-[(1S)-1-methoxyethyl]pyridin-3-yl}-6-(4,4,5,5-tetramethyl-1,3,2-dioxaborolan-2-yl)-1-azatricyclo[6.3.1.0^{4,12}]dodeca-2,4(12),5,7-tetraen-3-yl)-2,2-dimethylpropyl Acetate

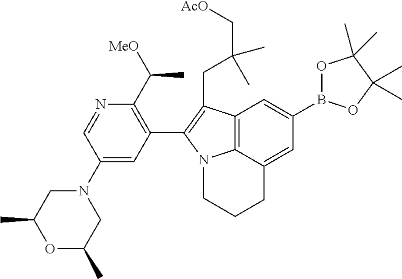

The title compound (300 mg, 84%) was prepared in a manner similar to that in example 9 step 3 from 3-(6-bromo-2-{5-[(2R,6S)-2,6-dimethylmorpholin-4-yl]-2-[(1S)-1-methoxyethyl]pyridin-3-yl}-1-azatricyclo[6.3.1.0^{4,12}]dodeca-2,4(12),5,7-tetraen-3-yl)-2,2-dimethylpropyl acetate and BPD. LC-MS (M+H)⁺=660.5.

### Step 3: methyl (4S)-2-[(2S)-3-(4-{3-[3-(acetyloxy)-2,2-dimethylpropyl]-2-{5-[(2R,6S)-2,6-dimethylmorpholin-4-yl]-2-[(1S)-1-methoxyethyl]pyridin-3-yl}-1-azatricyclo[6.3.1.0^{4,12}]dodeca-2,4(12),5,7-tetraen-6-yl}-1,3-thiazol-2-yl)-2-{[(tert-butoxy)carbonyl]amino}propanoyl]-2,3-diazabicyclo[3.1.1]heptane-4-carboxylate

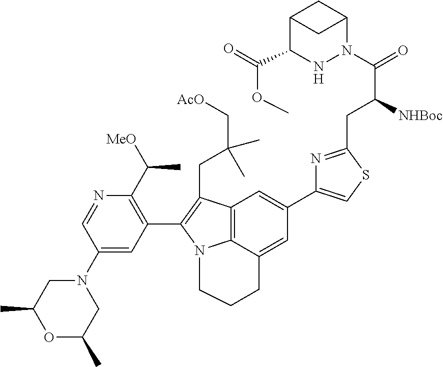 [SMILES: COC(=O)[C@H]1NN(C(=O)[C@H](Cc2nc(-c3cc4c5c(c3)c(CC(C)(C)COC(C)=O)c(-c3cc(N6C[C@@H](C)O[C@@H](C)C6)cnc3[C@H](C)OC)n5CCC4)cs2)NC(=O)OC(C)(C)C)C2CC1C2]

To a mixture of 3-(2-{5-[(2R,6S)-2,6-dimethylmorpholin-4-yl]-2-[(1S)-1-methoxyethyl]pyridin-3-yl}-6-(4,4,5,5-tetramethyl-1,3,2-dioxaborolan-2-yl)-1-azatricyclo[6.3.1.0412]dodeca-2,4(12),5,7-tetraen-3-yl)-2,2-dimethylpropyl acetate (200 mg, 0.303 mmol) and methyl (4S)-2-[(2S)-3-(4-bromo-1,3-thiazol-2-yl)-2-{[(tert-butoxy)carbonyl]amino}propanoyl]-2,3-diazabicyclo[3.1.1]heptane-4-carboxylate (148 mg, 0.303 mmol) in dioxane (4 mL) and water (0.5 mL) was added K₃PO₄ (128 mg, 0.606 mmol) and Pd(dtbpf)Cl₂ (20 mg, 0.03 mmol). The mixture was stirred for 8 h at 75° C. under nitrogen and then cooled to room temperature. The mixture was diluted with EA (50 mL), washed with water (20 mL), brine (20 mL). The organic phase was dried over Na₂SO₄, filtered and concentrated under vacuum. The residue was purified by prep-TLC (EA/PE=1/0) to give the title compound (200 mg, 70%). LC-MS (M+H)⁺=942.6.

### Step 4: (4S)-2-[(2S)-2-{[(tert-butoxy)carbonyl]amino}-3-[4-(2-{5-[(2R,6S)-2,6-dimethylmorpholin-4-yl]-2-[(1S)-1-methoxyethyl]pyridin-3-yl}-3-(3-hydroxy-2,2-dimethylpropyl)-1-azatricyclo[6.3.1.0^{4,12}]dodeca-2,4(12),5,7-tetraen-6-yl)-1,3-thiazol-2-yl]propanoyl]-2,3-diazabicyclo[3.1.1]heptane-4-carboxylic Acid

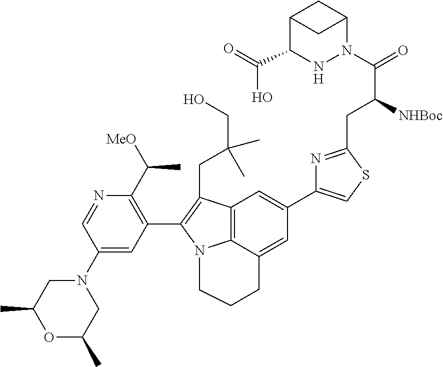 [SMILES: CO[C@@H](C)c1ncc(N2C[C@@H](C)O[C@@H](C)C2)cc1-c1c(CC(C)(C)CO)c2cc(-c3csc(C[C@H](NC(=O)OC(C)(C)C)C(=O)N4N[C@H](C(=O)O)C5CC4C5)n3)cc3c2n1CCC3]

To a mixture of methyl (4S)-2-[(2S)-3-(4-{3-[3-(acetyloxy)-2,2-dimethylpropyl]-2-{5-[(2R,6S)-2,6-dimethylmorpholin-4-yl]-2-[(1S)-1-methoxyethyl]pyridin-3-yl}-1-azatricyclo[6.3.1.0^{4,12}]dodeca-2,4(12),5,7-tetraen-6-yl}-1,3-thiazol-2-yl)-2-{[(tert-butoxy)carbonyl]amino}propanoyl]-2,3-diazabicyclo[3.1.1]heptane-4-carboxylate (200 mg, 0.21 mmol) in THF (5 mL) and water (2 mL) was added LiOH (36 mg, 1.5 mmol). The mixture was stirred overnight at room temperature. The mixture was diluted with water (30 mL) and the pH was adjusted to ˜5 with hydrochloric acid (0.5 N) at 0° C. The mixture was extracted with EA (40 mL×3). The combined organic layer was dried over Na₂SO₄, filtered and concentrated under vacuum to give the title compound (140 mg, 71%). LC-MS (M+H)⁺=886.5.

### Step 5: tert-butyl N-[(7S,13S)-20-{5-[(2R,6S)-2,6-dimethylmorpholin-4-yl]-2-[(1S)-1-methoxyethyl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate

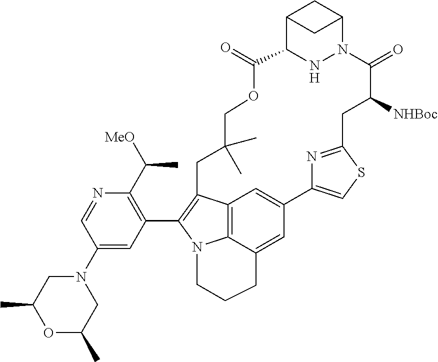 [SMILES: CO[C@@H](C)c1ncc(N2C[C@@H](C)O[C@@H](C)C2)cc1-c1c2c3cc(cc4c3n1CCC4)-c1csc(n1)C[C@H](NC(=O)OC(C)(C)C)C(=O)N1N[C@H](C(=O)OCC(C)(C)C2)C2CC1C2]

To a mixture of (4S)-2-[(2S)-2-{[(tert-butoxy)carbonyl]amino}-3-[4-(2-{5-[(2R,6S)-2,6-dimethylmorpholin-4-yl]-2-[(1S)-1-methoxyethyl]pyridin-3-yl}-3-(3-hydroxy-2,2-dimethylpropyl)-1-azatricyclo[6.3.1.0^{4,12}]dodeca-2,4(12),5,7-tetraen-6-yl)-1,3-thiazol-2-yl]propanoyl]-2,3-diazabicyclo[3.1.1]heptane-4-carboxylic acid (140 mg, 0.15 mmol) in MeCN (80 mL) was added NMI (0.37 g, 4.5 mmol) and TCFH (1.26 g, 4.5 mmol). The mixture was stirred for 1 h at room temperature and then concentrated under reduced pressure. The residue was re-dissolved in EA (40 mL) and successively washed with water (20 mL) and brine (20 mL). The organic layer was dried over Na₂SO₄, filtered and concentrated under vacuum. The residue was purified by prep-TLC (EA/PE=1/0) to give the title compound (50 mg, 39%). LC-MS (M+H)⁺=868.4.

### Step 6: (7S,13S)-7-amino-20-{5-[(2R,6S)-2,6-dimethylmorpholin-4-yl]-2-[(1S)-1-methoxyethyl]pyridin-3-yl}-17,17-dimethyl-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione

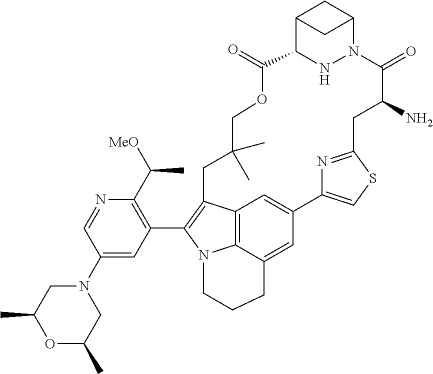 [SMILES: CO[C@@H](C)c1ncc(N2C[C@@H](C)O[C@@H](C)C2)cc1-c1c2c3cc(cc4c3n1CCC4)-c1csc(n1)C[C@H](N)C(=O)N1N[C@H](C(=O)OCC(C)(C)C2)C2CC1C2]

To a mixture of tert-butyl N-[(7S,13S)-20-{5-[(2R,6S)-2,6-dimethylmorpholin-4-yl]-2-[(1S)-1-methoxyethyl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate (50 mg, 0.057 mmol) in MeOH (1 mL) was added HCl in dioxane (4.0 M, 4 mL). The mixture was stirred for 1 h at room temperature and concentrated under vacuum. The residue was partitioned between DCM (10 mL) and saturated NaHCO₃ (5 mL). The organic layer was dried over Na₂SO₄, filtered and concentrated under vacuum to give the title compound (35 mg, 80%). LC-MS (M+H)⁺=768.5.

### Step 7: ((1R,2R,3S)—N-[(7S,13S,19P)-20-{5-[(2R,6S)-2,6-dimethylmorpholin-4-yl]-2-[(1S)-1-methoxyethyl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide & (1R,2R,3S)—N-[(7S,13S,19M)-20-{5-[(2R,6S)-2,6-dimethylmorpholin-4-yl]-2-[(1S)-1-methoxyethyl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

To a mixture of (7S,13S)-7-amino-20-{5-[(2R,6S)-2,6-dimethylmorpholin-4-yl]-2-[(1S)-1-methoxyethyl]pyridin-3-yl}-17,17-dimethyl-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione (20 mg, 0.026 mmol) in DMF (3 mL) was added DIPEA (16.8 mg, 0.13 mmol), (1r,2R,3S)-2,3-dimethylcyclopropane-1-carboxylic acid (5.9 mg, 0.052 mmol) and HATU (14.8 mg, 0.039 mmol). The mixture was stirred at room temperature for 1 h and then diluted with EA (40 mL). The mixture was washed with brine (20 mL×3), dried over Na₂SO₄, filtered and concentrated under vacuum. The residue was purified by prep-TLC (DCM:MeOH=10:1) followed by prep-HPLC to give Atrop-16 (P-isomer, 0.5 mg, 2%) and Example-16 (M-isomer, 1.6 mg, 7%).

Atrop-16: ¹H NMR (400 MHz, DMSO-d6) δ 8.47-8.43 (m, 2H), 8.27 (s, 1H), 7.73 (s, 1H), 7.44 (s, 1H), 7.39 (d, J=2.7 Hz, 1H), 5.88 (d, J=11.0 Hz, 1H), 5.31 (t, J=8.1 Hz, 2H), 4.75 (d, J=11.0 Hz, 1H), 4.50 (d, J=4.7 Hz, 1H), 4.00-3.90 (m, 3H), 3.76-3.61 (m, 5H), 3.10 (s, 4H), 2.98 (d, J=6.4 Hz, 3H), 2.67 (d, J=1.8 Hz, 3H), 2.38-2.30 (m, 4H), 2.22-2.13 (m, 2H), 2.02-1.97 (m, 3H), 1.62 (t, J=9.1 Hz, 1H), 1.18-1.10 (m, 11H), 1.09-1.06 (m, 4H), 0.94 (s, 3H), 0.87-0.84 (m, 1H), 0.52 (s, 3H). LC-MS (M+H)⁺=864.7.

Example-16: ¹H NMR (400 MHz, DMSO-d6) δ 8.45-8.31 (m, 2H), 8.20 (s, 1H), 7.68 (s, 1H), 7.39 (s, 1H), 7.13 (d, J=2.9 Hz, 1H), 5.89 (d, J=11.0 Hz, 1H), 5.50-5.29 (m, 1H), 4.62 (d, J=10.9 Hz, 1H), 4.52-4.36 (m, 1H), 4.33-4.04 (m, 2H), 3.81-3.45 (m, 7H), 3.23-3.14 (m, 4H), 3.08 (dd, J=14.8, 7.4 Hz, 1H), 3.00-2.95 (m, 1H), 2.87-2.82 (m, 2H), 2.67-2.55 (m, 2H), 2.33-1.90 (m, 7H), 1.51 (t, J=9.3 Hz, 1H), 1.30 (d, J=6.1 Hz, 3H), 1.23-0.99 (m, 15H), 0.84 (s, 3H), 0.35 (s, 3H). LC-MS (M+H)⁺=864.7.

Atrop-17 & Example 17: (1S,2S)—N-[(7S,13S,19P)-20-{5-[(2R,6S)-2,6-dimethylmorpholin-4-yl]-2-[(1S)-1-methoxyethyl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2-methylcyclopropane-1-carboxamide & (1S,2S)—N-[(7S,13S,19M)-20-{5-[(2R,6S)-2,6-dimethylmorpholin-4-yl]-2-[(1S)-1-methoxyethyl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2-methylcyclopropane-1-carboxamide

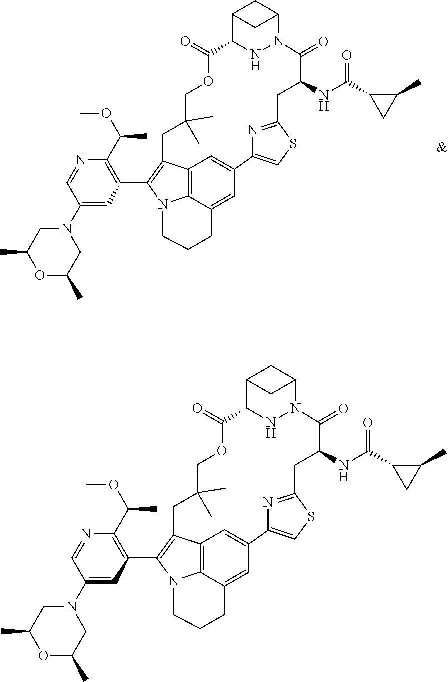

Atrop-17 (P-isomer, 0.4 mg, 2%) and example 17 (M-isomer, 1.4 mg, 6%) were prepared in a manner similar to that in example 16 step 6 from (7S,13S)-7-amino-20-{5-[(2R,6S)-2,6-dimethylmorpholin-4-yl]-2-[(1S)-1-methoxyethyl]pyridin-3-yl}-17,17-dimethyl-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione and (1S,2S)-2-methylcyclopropane-1-carboxylic acid, and the atropisomers were separated by prep-HPLC.

Atrop-17: ¹H NMR (400 MHz, DMSO-d6) δ 8.56 (d, J=8.7 Hz, 1H), 8.44 (s, 1H), 8.27 (s, 1H), 7.73 (s, 1H), 7.44 (s, 1H), 7.39 (s, 1H), 5.90 (d, J=11.0 Hz, 1H), 5.32 (d, J=7.1 Hz, 1H), 4.75 (d, J=11.0 Hz, 1H), 4.53-4.49 (m, 1H), 4.00-3.95 (m, 1H), 3.92-3.87 (m, 1H), 3.74-3.68 (m, 4H), 3.63 (d, J=10.4 Hz, 4H), 3.10 (s, 3H), 3.03-2.98 (m, 3H), 2.67 (s, 2H), 2.37-2.31 (m, 3H), 2.18-2.13 (m, 1H), 2.03-1.97 (m, 3H), 1.63 (t, J=8 Hz, 1H), 1.52-1.43 (m, 2H), 1.15-1.15 (m, 8H), 1.07 (s, 4H), 0.94 (s, 3H), 0.85 (t, J=6.6 Hz, 2H), 0.57 (t, J=4 Hz, 1H), 0.52 (s, 3H). LC-MS (M+H)⁺=850.7.

Example 17: ¹H NMR (400 MHz, DMSO-d6) δ 8.50 (d, J=8.9 Hz, 1H), 8.39 (d, J=3.0 Hz, 1H), 8.20 (s, 1H), 7.67 (s, 1H), 7.39 (s, 1H), 7.13 (d, J=2.9 Hz, 1H), 5.90 (d, J=11.0 Hz, 1H), 5.34 (t, J=8.1 Hz, 1H), 4.63 (d, J=11.0 Hz, 1H), 4.45 (q, J=4.9 Hz, 1H), 4.32-3.94 (m, 2H), 3.75-3.44 (m, 7H), 3.24-3.13 (m, 4H), 3.07 (dd, J=14.9, 7.3 Hz, 1H), 3.01-2.93 (m, 1H), 2.91-2.80 (m, 2H), 2.63-2.56 (m, 1H), 2.37-1.88 (m, 8H), 1.52 (t, J=9.3 Hz, 1H), 1.47-1.41 (m, 1H), 1.30 (d, J=6.0 Hz, 3H), 1.15-1.00 (m, 10H), 0.85 (s, 4H), 0.57-0.48 (m, 1H), 0.35 (s, 3H). LC-MS (M+H)⁺=850.7.

Atrop-18 & Example 18: (1R,2R,3S)—N-[(7S,13S,19P)-20-{2-[(1S)-1-methoxyethyl]-5-{5-methyl-4-oxa-7-azaspiro[2.5]octan-7-yl}pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide and (1R,2R,3S)—N-[(7S,13S,19M)-20-{2-[(1S)-1-methoxyethyl]-5-{5-methyl-4-oxa-7-azaspiro[2.5]octan-7-yl}pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

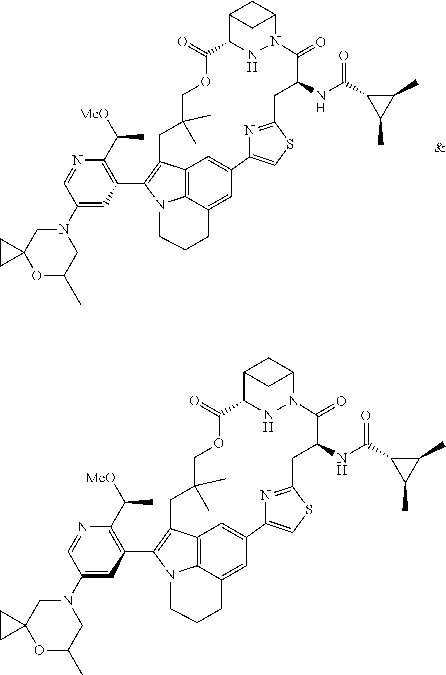

### Step 1: 2-bromo-N-[(1-hydroxycyclopropyl)methyl]propanamide

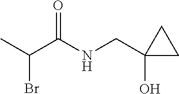

To a mixture of 1-(aminomethyl)cyclopropan-1-ol (1.0 g, 11.5 mmol) and Et₃N (3.48 g, 34.5 mmol) in anhydrous DCM (20 mL) was added 2-bromopropanoyl chloride (1.97 g, 11.5 mmol) dropwise at 0° C. The mixture was stirred at room temperature for 2 h and concentrated under reduced pressure. The residue was purified by silica gel chromatograph (EA/PE=1/10 to 6/4) to give the title compound (1.59 g, 63%). LC-MS (M+H)⁺=222.4.

### Step 2: 5-methyl-4-oxa-7-azaspiro[2.5]octan-6-one

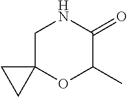

To a mixture of 2-bromo-N-[(1-hydroxycyclopropyl)methyl]propanamide (1.59 g, 7.19 mmol) in anhydrous THF (15 mL) was added NaH (0.43 g, 11 mmol) at 0° C. The reaction mixture was allowed to warm to room temperature and stirred at room temperature for 2 h. The reaction mixture was quenched with ice-water (0.3 mL). Then, the mixture was dried over Na₂SO₄, filtered and concentrated under vacuum to give a residue. The residue was purified by flash-column chromatography on silica gel (EA:PE=0:1 to 8:2) to give the title compound (1.0 g, 989%). LC-MS (M+H)⁺=142.4.

### Step 3: 5-methyl-4-oxa-7-azaspiro[2.5]octane

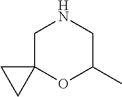

To a mixture of 5-methyl-4-oxa-7-azaspiro[2.5]octan-6-one (1.0 g, 7.09 mmol) in anhydrous THF (10 mL) was added LiAlH₄ (2.5 M in THF, 5.7 mL, 14.2 mmol) dropwise at 0° C. and the mixture was stirred at room temperature for 6 h. The mixture was cooled to 0° C. and EA (70 mL) was added dropwise. Na₂SO₄·10H₂O (20 g) was added to the mixture, and the mixture was stirred at room temperature for 1 h. Solid was filtered off and the filtrate was dried over Na₂SO₄, filtered and concentrated under vacuum to give the title compound (0.65 g, 72%). LC-MS (M+H)⁺=128.3.

### Step 4: 3-(6-bromo-2-{2-[(1S)-1-methoxyethyl]-5-{5-methyl-4-oxa-7-azaspiro[2.5]octan-7-yl}pyridin-3-yl}-1-azatricyclo[6.3.1.0^{4,12}]dodeca-2,4(12),5,7-tetraen-3-yl)-2,2-dimethylpropyl Acetate

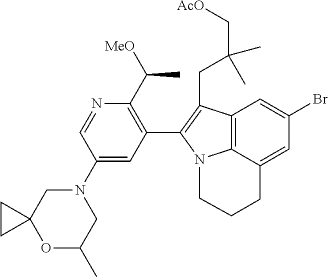 [SMILES: CO[C@@H](C)c1ncc(N2CC(C)OC3(CC3)C2)cc1-c1c(CC(C)(C)COC(C)=O)c2cc(Br)cc3c2n1CCC3]

To a mixture of (5-{3-[3-(acetyloxy)-2,2-dimethylpropyl]-6-bromo-1-azatricyclo[6.3.1.0^{4,12}]dodeca-2,4(12),5,7-tetraen-2-yl}-6-[(1S)-1-methoxyethyl]pyridin-3-yl)boronic acid (500 mg, 0.92 mmol) in MeCN (8 mL) was added 5-methyl-4-oxa-7-azaspiro[2.5]octane (586 mg, 4.6 mmol), Cu(OAc)₂ (335 mg, 1.84 mmol) and Et₃N (279 mg, 2.76 mmol). The mixture was stirred at room temperature overnight under oxygen and then treated with brine (50 mL). Solid was filtered off, and the filter cake was rinsed with DCM:MeOH (10:1, 300 mL). The organic layer of the filtrate was separated, dried over Na₂SO₄, filtered and concentrated under vacuum. The residue was purified by prep-TLC (EA) to give the title compound (320 mg, 55%). LC-MS (M+H)⁺=624.5.

### Step 5: 3-(2-{2-[(1S)-1-methoxyethyl]-5-{5-methyl-4-oxa-7-azaspiro[2.5]octan-7-yl}pyridin-3-yl}-6-(4,4,5,5-tetramethyl-1,3,2-dioxaborolan-2-yl)-1-azatricyclo[6.3.1.0^{4,12}]dodeca-2,4(12),5,7-tetraen-3-yl)-2,2-dimethylpropyl Acetate

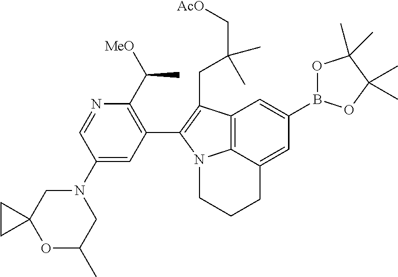 [SMILES: CO[C@@H](C)c1ncc(N2CC(C)OC3(CC3)C2)cc1-c1c(CC(C)(C)COC(C)=O)c2cc(B3OC(C)(C)C(C)(C)O3)cc3c2n1CCC3]

To a mixture of 3-(6-bromo-2-{2-[(1S)-1-methoxyethyl]-5-{5-methyl-4-oxa-7-azaspiro[2.5]octan-7-yl}pyridin-3-yl}-1-azatricyclo[6.3.1.0^{4,12}]dodeca-2,4(12),5,7-tetraen-3-yl)-2,2-dimethylpropyl acetate (320 mg, 0.51 mmol) and BPD (330 mg, 1.28 mmol) in dioxane (6 mL) was added Pd(dppf)Cl₂ (38 mg, 0.0514 mmol) and KOAc (150 mg, 1.54 mmol). The mixture was stirred at 90° C. for 1 h under nitrogen and then cooled to room temperature. The mixture was diluted with EA (50 mL) and washed with brine (40 mL). The organic layer was dried over Na₂SO₄, filtered and concentrated under vacuum. The crude product was purified by silica gel chromatograph (EA:PE=0:1 to 4:1) to give the title compound (230 mg, 66%). LC-MS (M+H)⁺=672.6.

### Step 6: methyl (4S)-2-[(2S)-3-(4-{3-[3-(acetyloxy)-2,2-dimethylpropyl]-2-{2-[(1S)-1-methoxyethyl]-5-{5-methyl-4-oxa-7-azaspiro[2.5]octan-7-yl}pyridin-3-yl}-1-azatricyclo[6.3.1.0^{4,12}]dodeca-2,4(12),5,7-tetraen-6-yl}-1,3-thiazol-2-yl)-2-{[(tert-butoxy)carbonyl]amino}propanoyl]-2,3-diazabicyclo[3.1.1]heptane-4-carboxylate

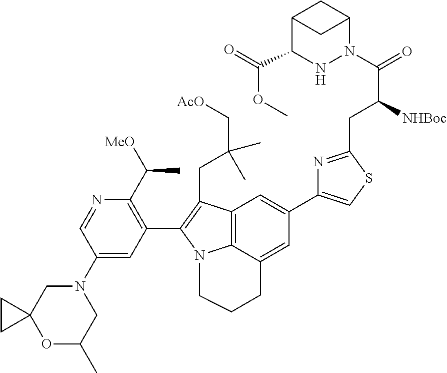 [SMILES: COC(=O)[C@H]1NN(C(=O)[C@H](Cc2nc(-c3cc4c5c(c3)c(CC(C)(C)COC(C)=O)c(-c3cc(N6CC(C)OC7(CC7)C6)cnc3[C@H](C)OC)n5CCC4)cs2)NC(=O)OC(C)(C)C)C2CC1C2]

The title compound (69 mg, 21%) was prepared in a manner similar to that in example 16 step 3 from 3-(2-{2-[(1S)-1-methoxyethyl]-5-{5-methyl-4-oxa-7-azaspiro[2.5]octan-7-yl}pyridin-3-yl}-6-(4,4,5,5-tetramethyl-1,3,2-dioxaborolan-2-yl)-1-azatricyclo[6.3.1.0^{4,12}]dodeca-2,4(12),5,7-tetraen-3-yl)-2,2-dimethylpropyl acetate and methyl (4S)-2-[(2S)-3-(4-bromo-1,3-thiazol-2-yl)-2-{[(tert-butoxy)carbonyl]amino}propanoyl]-2,3-diazabicyclo[3.1.1]heptane-4-carboxylate. LC-MS (M+H)⁺=954.4.

### Step 7: (4S)-2-[(2S)-2-{[(tert-butoxy)carbonyl]amino}-3-{4-[3-(3-hydroxy-2,2-dimethylpropyl)-2-{2-[(1S)-1-methoxyethyl]-5-{5-methyl-4-oxa-7-azaspiro[2.5]octan-7-yl}pyridin-3-yl}-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic Acid

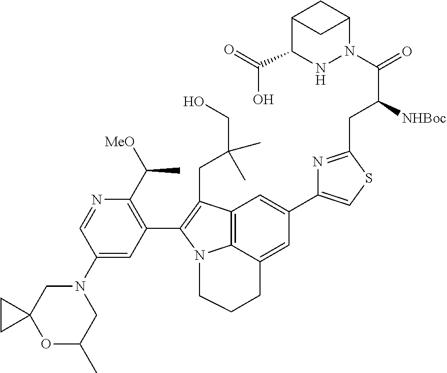 [SMILES: CO[C@@H](C)c1ncc(N2CC(C)OC3(CC3)C2)cc1-c1c(CC(C)(C)CO)c2cc(-c3csc(C[C@H](NC(=O)OC(C)(C)C)C(=O)N4N[C@H](C(=O)O)C5CC4C5)n3)cc3c2n1CCC3]

The title compound (47 mg, 72%) was prepared in a manner similar to that in example 16 step 4 from methyl (4S)-2-[(2S)-3-(4-{3-[3-(acetyloxy)-2,2-dimethylpropyl]-2-{2-[(1S)-1-methoxyethyl]-5-{5-methyl-4-oxa-7-azaspiro[2.5]octan-7-yl}pyridin-3-yl}-1-azatricyclo[6.3.1.0^{4,12}]dodeca-2,4(12),5,7-tetraen-6-yl}-1,3-thiazol-2-yl)-2-{[(tert-butoxy)carbonyl]amino}propanoyl]-2,3-diazabicyclo[3.1.1]heptane-4-carboxylate. LC-MS (M+H)⁺=898.5.

### Step 8: tert-butyl N-[(7S,13S)-20-{2-[(1S)-1-methoxyethyl]-5-{5-methyl-4-oxa-7-azaspiro[2.5]octan-7-yl}pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate

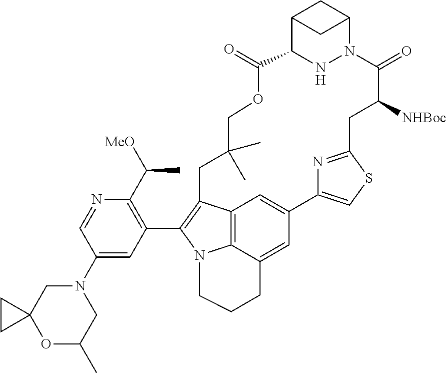 [SMILES: CO[C@@H](C)c1ncc(N2CC(C)OC3(CC3)C2)cc1-c1c2c3cc(cc4c3n1CCC4)-c1csc(n1)C[C@H](NC(=O)OC(C)(C)C)C(=O)N1N[C@H](C(=O)OCC(C)(C)C2)C2CC1C2]

The title compound (29 mg, 51%) was prepared in a manner similar to that in example 16 step 5 from (4S)-2-[(2S)-2-{[(tert-butoxy)carbonyl]amino}-3-{4-[3-(3-hydroxy-2,2-dimethylpropyl)-2-{2-[(1S)-1-methoxyethyl]-5-{5-methyl-4-oxa-7-azaspiro[2.5]octan-7-yl}pyridin-3-yl}-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic acid. LC-MS (M+H)⁺=880.3.

### Step 9: (7S,13S)-7-amino-20-{2-[(1S)-1-methoxyethyl]-5-{5-methyl-4-oxa-7-azaspiro[2.5]octan-7-yl}pyridin-3-yl}-17,17-dimethyl-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione

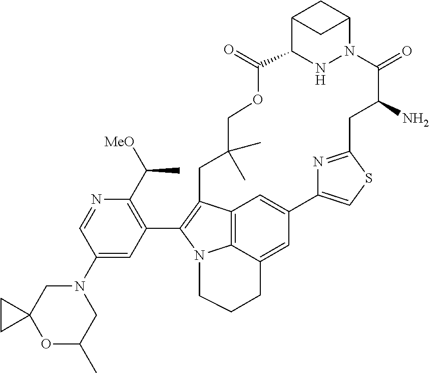 [SMILES: CO[C@@H](C)c1ncc(N2CC(C)OC3(CC3)C2)cc1-c1c2c3cc(cc4c3n1CCC4)-c1csc(n1)C[C@H](N)C(=O)N1N[C@H](C(=O)OCC(C)(C)C2)C2CC1C2]

The title compound (20 mg, 79%) was prepared in a manner similar to that in example 16 step 6 from tert-butyl N-[(7S,13S)-20-{2-[(1S)-1-methoxyethyl]-5-{5-methyl-4-oxa-7-azaspiro[2.5]octan-7-yl}pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate. LC-MS (M+H)⁺=780.5.

### Step 10: (1R,2R,3S)—N-[(7S,13S,19P)-20-{2-[(1S)-1-methoxyethyl]-5-{5-methyl-4-oxa-7-azaspiro[2.5]octan-7-yl}pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide and (1R,2R,3S)—N-[(7S,13S,19M)-20-{2-[(1S)-1-methoxyethyl]-5-{5-methyl-4-oxa-7-azaspiro[2.5]octan-7-yl}pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

Atrop-18 (P-isomer, 0.4 mg, 2%) and Example 18 (M-isomer, 0.4 mg, 2%) were prepared in a manner similar to that in example 16 step 7 from (7S,13S)-7-amino-20-{2-[(1S)-1-methoxyethyl]-5-{5-methyl-4-oxa-7-azaspiro[2.5]octan-7-yl}pyridin-3-yl}-17,17-dimethyl-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione and (1r,2R,3S)-2,3-dimethylcyclopropane-1-carboxylic acid, and the atropisomers were separated by prep-HPLC.

Atrop-18: ¹H NMR (400 MHz, DMSO-d6) δ 8.45-4.39 (m, 2H), 8.27 (s, 1H), 7.73 (s, 1H), 7.44 (s, 1H), 7.39 (t, J=4 Hz, 1H), 5.89 (d, J=11.2 Hz, 1H), 5.31 (t, J=8.2 Hz, 1H), 4.75 (d, J=11.0 Hz, 1H), 4.52-4.49 (m, 1H), 4.00-3.95 (m, 1H), 3.94-3.76 (m, 5H), 3.63 (d, J=10.7 Hz, 2H), 3.10 (s, 4H), 2.98 (d, J=6.4 Hz, 3H), 2.67 (d, J=4.4 Hz, 2H), 2.42-2.33 (m, 3H), 2.19-2.12 (m, 3H), 2.04-1.96 (m, 2H), 1.62 (t, J=9.2 Hz, 1H), 1.14-1.04 (m, 13H), 0.94 (s, 3H), 0.89-0.76 (m, 3H), 0.67-0.62 (m, 3H), 0.52 (s, 3H). LC-MS (M+H)⁺=876.4.

Example 18: ¹H NMR (400 MHz, DMSO-d6) δ 8.47-8.37 (m, 2H), 8.24 (s, 1H), 7.72 (s, 1H), 7.44 (s, 1H), 7.18 (d, J=2.4 Hz, 1H), 5.94 (d, J=10.8 Hz, 1H), 5.38-5.32 (m, 1H), 4.69-4.65 (m, 1H), 4.51-4.47 (m, 1H), 4.30-4.26 (m, 1H), 4.23-4.17 (m, 1H), 3.86-3.78 (m, 2H), 3.61 (d, J=10.6 Hz, 2H), 3.57-3.50 (m, 3H), 3.23-3.20 (m, 5H), 3.16-3.10 (m, 2H), 3.02 (t, J=4 Hz, 1H), 2.94-2.88 (m, 2H), 2.69-2.62 (m, 2H), 2.35-2.30 (m, 1H), 2.18-2.09 (m, 2H), 2.03-1.97 (m, 1H), 1.58-1.52 (m, 1H), 1.35 (d, J=5.9 Hz, 3H), 1.17-1.06 (m, 12H), 0.92-0.87 (m, 3H), 0.86-0.76 (m, 2H), 0.63-0.59 (m, 2H), 0.40 (s, 3H). LC-MS (M+H)⁺=876.4.

Atrop-19 & Example 19: (1R,2R,3S)—N-[(7S,13S,19P)-20-{2-[(1S)-1-methoxyethyl]-5-{4-methylpiperazin-1-yl}pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide & (1R,2R,3S)—N-[(7S,13S,19M)-20-{2-[(1S)-1-methoxyethyl]-5-(4-methylpiperazin-1-yl)pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

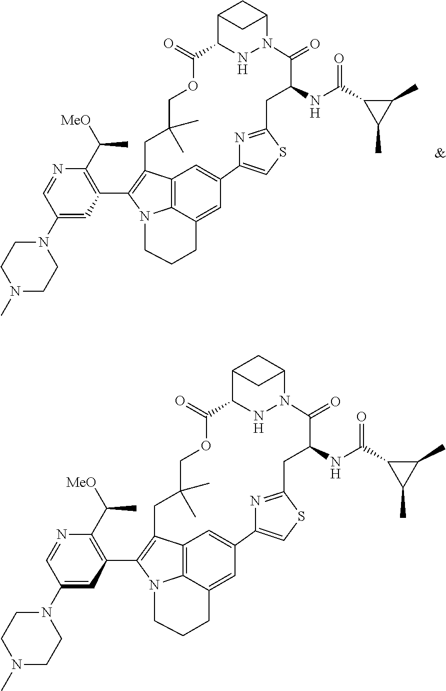

### Step 1: 3-(6-bromo-2-{2-[(1S)-1-methoxyethyl]-5-(4-methylpiperazin-1-yl)pyridin-3-yl}-1-azatricyclo[6.3.1.0^{4,12}]dodeca-2,4(12),5,7-tetraen-3-yl)-2,2-dimethylpropyl Acetate

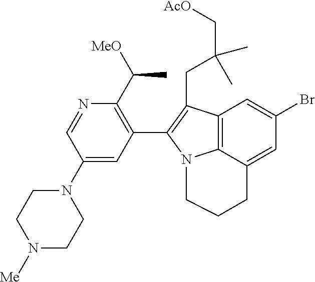

To a mixture of 3-(6-bromo-2-{2-[(1S)-1-methoxyethyl]-5-(4,4,5,5-tetramethyl-1,3,2-dioxaborolan-2-yl)pyridin-3-yl}-1-azatricyclo[6.3.1.0^{4,12}]dodeca-2,4(12),5,7-tetraen-3-yl)-2,2-dimethylpropyl acetate (1 g, 1.6 mmol) and 1-methylpiperazine (802 mg, 8 mmol) in MeCN (30 mL) was added TEA (485 mg, 4.8 mmol) and Cu(OAc)₂ (582 mg, 3.2 mmol). The mixture was stirred for 4 h at room temperature under oxygen atmosphere. The mixture was then diluted with EA (100 mL) and water (100 mL). The solid was filtered off and the filter cake was rinsed with EA (2×30 mL). The organic phase was separated and washed with water (50 mL), brine (60 mL), dried over Na₂SO₄, filtered and concentrated under vacuum. The residue was purified by silica gel column chromatography (DCM:MeOH=15:1) to give the title compound (465 mg, 49%). LC-MS (M+H)⁺=597.3/599.3.

### Step 2: 3-(2-{2-[(1S)-1-methoxyethyl]-5-(4-methylpiperazin-1-yl)pyridin-3-yl}-6-(4,4,5,5-tetramethyl-1,3,2-dioxaborolan-2-yl)-1-azatricyclo[6.3.1.0^{4,12}]dodeca-2,4(12),5,7-tetraen-3-yl)-2,2-dimethylpropyl Acetate

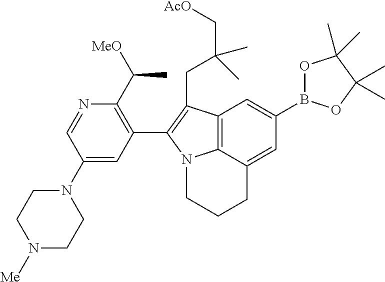 [SMILES: CO[C@@H](C)c1ncc(N2CCN(C)CC2)cc1-c1c(CC(C)(C)COC(C)=O)c2cc(B3OC(C)(C)C(C)(C)O3)cc3c2n1CCC3]

To a mixture of 3-(6-bromo-2-(2-[(1S)-1-methoxyethyl]-5-{4-methylpiperazin-1-yl)pyridin-3-yl}-1-azatricyclo[6.3.1.0^{4,12}]dodeca-2,4(12),5,7-tetraen-3-yl)-2,2-dimethylpropyl acetate (465 mg, 0.78 mmol) and BPD (495 mg, 1.95 mmol) in toluene (10 mL) was added Pd(dppf)Cl₂·DCM (64 mg, 0.078 mmol) and KOAc (229 mg, 2.34 mmol). The mixture was stirred for 4 h at 90° C. under nitrogen. The mixture was cooled to room temperature and then diluted with EA (40 mL), washed with brine (20 mL). The organic phase was dried over Na₂SO₄, filtered and concentrated under vacuum. The residue was purified by prep-TLC (DCM:MeOH=15:1) to give the title compound (430 mg, 86%). LC-MS (M+H)⁺=645.5.

### Step 3: methyl (4S)-2-[(2S)-3-(4-{3-[3-(acetyloxy)-2,2-dimethylpropyl]-2-{2-[(1S)-1-methoxyethyl]-5-(4-methylpiperazin-1-yl)pyridin-3-yl}-1-azatricyclo[6.3.1.0^{4,12}]dodeca-2,4(12),5,7-tetraen-6-yl}-1,3-thiazol-2-yl)-2-{[(tert-butoxy)carbonyl]amino}propanoyl]-2,3-diazabicyclo[3.1.1]heptane-4-carboxylate

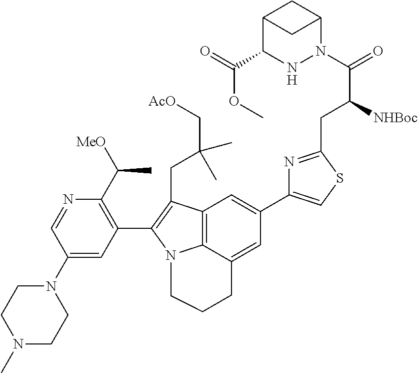 [SMILES: COC(=O)[C@H]1NN(C(=O)[C@H](Cc2nc(-c3cc4c5c(c3)c(CC(C)(C)COC(C)=O)c(-c3cc(N6CCN(C)CC6)cnc3[C@H](C)OC)n5CCC4)cs2)NC(=O)OC(C)(C)C)C2CC1C2]

To a mixture of 3-(2-{2-[(1S)-1-methoxyethyl]-5-(4-methylpiperazin-1-yl)pyridin-3-yl}-6-(4,4,5,5-tetramethyl-1,3,2-dioxaborolan-2-yl)-1-azatricyclo[6.3.1.0^{4,12}]dodeca-2,4(12),5,7-tetraen-3-yl)-2,2-dimethylpropyl acetate (367 mg, 0.57 mmol) and methyl (4S)-2-[(2S)-3-(4-bromo-1,3-thiazol-2-yl)-2-{[(tert-butoxy)carbonyl]amino}propanoyl]-2,3-diazabicyclo[3.1.1]heptane-4-carboxylate (280 mg, 0.57 mmol) in dioxane (8 mL) and water (1 mL) was added K₃PO₄ (240 mg, 2.01 mmol) and Pd(dtbpf)Cl₂ (37 mg, 0.057 mmol). The mixture was stirred at 75° C. for 8 h under nitrogen and then cooled to room temperature. The mixture was diluted with EA (50 mL) and successively washed with water (20 mL) and brine (20 mL). The organic layer was dried over Na₂SO₄, filtered and concentrated under vacuum. The residue was purified by prep-TLC (DCM:MeOH=10:1) to give the title compound (320 mg, 60.7%). LC-MS (M+H)⁺=927.4.

### Step 4: (4S)-2-[(2S)-2-{[(tert-butoxy)carbonyl]amino}-3-{4-[3-(3-hydroxy-2,2-dimethylpropyl)-2-{2-[(1S)-1-methoxyethyl]-5-(4-methylpiperazin-1-yl)pyridin-3-yl}-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic Acid

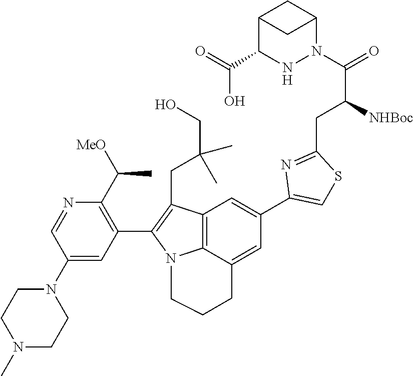 [SMILES: CO[C@@H](C)c1ncc(N2CCN(C)CC2)cc1-c1c(CC(C)(C)CO)c2cc(-c3csc(C[C@H](NC(=O)OC(C)(C)C)C(=O)N4N[C@H](C(=O)O)C5CC4C5)n3)cc3c2n1CCC3]

To a mixture of methyl (4S)-2-[(2S)-3-(4-{3-[3-(acetyloxy)-2,2-dimethylpropyl]-2-{2-[(1S)-1-methoxyethyl]-5-(4-methylpiperazin-1-yl)pyridin-3-yl}-1-azatricyclo[6.3.1.0^{4,12}]dodeca-2,4(12),5,7-tetraen-6-yl}-1,3-thiazol-2-yl)-2-{[(tert-butoxy)carbonyl]amino}propanoyl]-2,3-diazabicyclo[3.1.1]heptane-4-carboxylate (320 mg, 0.346 mmol) in THF (6 mL) and water (6 mL) was added LiOH (58 mg, 2.42 mmol). The mixture was stirred at room temperature for 6 h. The mixture was diluted with water (4 mL) and washed with MTBE (20 mL×3). The pH of the aqueous phase was adjusted to pH ˜6 with hydrochloric acid (0.5 N) at 0° C. The mixture was extracted with DCM (30 mL×3). The combined organic layer was dried over Na₂SO₄, filtered and concentrated under reduced pressure to give the title compound (280 mg, 92%). LC-MS (M+H)⁺=871.4.

### Step 5: tert-butyl N-[(7S,13S)-20-{2-[(S)-1-methoxyethyl]-5-(4-methylpiperazin-1-yl)pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate

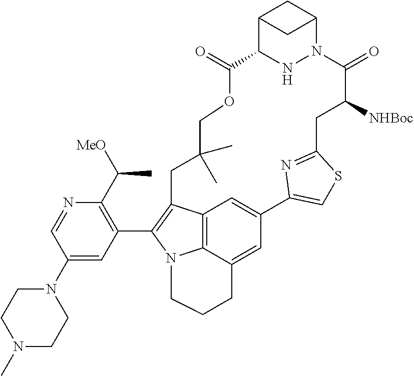 [SMILES: CO[C@@H](C)c1ncc(N2CCN(C)CC2)cc1-c1c2c3cc(cc4c3n1CCC4)-c1csc(n1)C[C@H](NC(=O)OC(C)(C)C)C(=O)N1N[C@H](C(=O)OCC(C)(C)C2)C2CC1C2]

The title compound (120 mg, 44%) was prepared in a manner similar to that in example 16 step 5 from (4S)-2-[(2S)-2-{[(tert-butoxy)carbonyl]amino}-3-{4-[3-(3-hydroxy-2,2-dimethylpropyl)-2-{2-[(1S)-1-methoxyethyl]-5-(4-methylpiperazin-1-yl)pyridin-3-yl}-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic acid. LC-MS (M+H)⁺=853.4.

### Step 6: (7S,13S)-7-amino-20-{2-[(1S)-1-methoxyethyl]-5-(4-methylpiperazin-1-yl)pyridin-3-yl}-17,17-dimethyl-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione

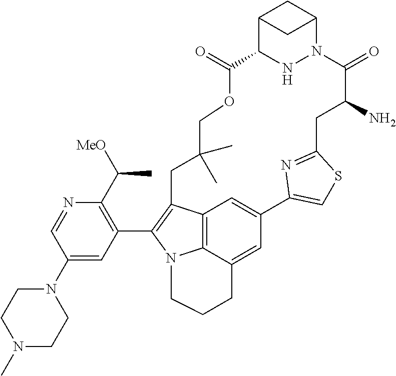 [SMILES: CO[C@@H](C)c1ncc(N2CCN(C)CC2)cc1-c1c2c3cc(cc4c3n1CCC4)-c1csc(n1)C[C@H](N)C(=O)N1N[C@H](C(=O)OCC(C)(C)C2)C2CC1C2]

The title compound (100 mg, 98%) was prepared in a manner similar to that in example 16 step 6 from tert-butyl N-[(7S,13S)-20-{2-[(1S)-1-methoxyethyl]-5-(4-methylpiperazin-1-yl)pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate. LC-MS (M+H)⁺=753.4.

### Step 7: (1R,2R,3S)—N-[(7S,13S,19P)-20-{2-[(1S)-1-methoxyethyl]-5-(4-methylpiperazin-1-yl)pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide & (1R,2R,3S)—N-[(7S,13S,19M)-20-{2-[(1S)-1-methoxyethyl]-5-(4-methylpiperazin-1-yl)pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

Atrop-19 (P-isomer, 11.3 mg, 10%) and example 19 (M-isomer, 10.8 mg, 10%) were prepared in a manner similar to that in example 16 step 7 from (7S,13S)-7-amino-20-(2-[(1S)-1-methoxyethyl]-5-{4-methylpiperazin-1-yl)pyridin-3-yl}-17,17-dimethyl-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione and (1r,2R,3S)-2,3-dimethylcyclopropane-1-carboxylic acid, and the atropisomers were separated by prep-HPLC

Atrop-19: ¹H NMR (400 MHz, DMSO-d6) ¹H NMR (500 MHz, DMSO) δ 8.60-8.38 (m, 2H), 8.27 (d, J=3.3 Hz, 1H), 7.73 (d, J=3.3 Hz, 1H), 7.51-7.23 (m, 2H), 5.97-5.78 (m, 1H), 5.42-5.19 (m, 1H), 4.86-4.68 (m, 1H), 4.55-4.44 (m, 1H), 4.10-3.80 (m, 2H), 3.69-3.44 (m, 3H), 3.26-2.93 (m, 12H), 2.79-2.61 (m, 4H), 2.41-1.93 (m, 10H), 1.63 (t, J=9.1 Hz, 1H), 1.29-1.01 (m, 12H), 0.94 (s, 3H), 0.53 (s, 3H). LC-MS (M+H)⁺=849.7.

Example 19: ¹H NMR (400 MHz, DMSO-d6) δ 8.55-8.36 (m, 2H), 8.23 (s, 1H), 7.71 (s, 1H), 7.42 (s, 1H), 7.16 (s, 1H), 5.92 (d, J=11.1 Hz, 1H), 5.36 (t, J=8.1 Hz, 1H), 4.66 (d, J=11.0 Hz, 1H), 4.56-4.40 (m, 1H), 4.34-4.11 (m, 2H), 3.70-3.42 (m, 3H), 3.29-3.18 (m, 8H), 3.10 (dd, J=14.9, 7.5 Hz, 1H), 3.04-2.96 (m, 1H), 2.93-2.84 (m, 2H), 2.69-2.57 (m, 4H), 2.44-2.08 (m, 10H), 1.54 (t, J=9.3 Hz, 1H), 1.33 (s, 3H), 1.22-0.99 (m, 9H), 0.87 (s, 3H), 0.37 (s, 3H). LC-MS (M+H)⁺=849.7.

### Example 20: (1R,2R,3S)—N-[(7S,13S,19M)-20-{2-[(1S)-1-methoxyethyl]-5-[(3R,5S)-3,4,5-trimethylpiperazin-1-yl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

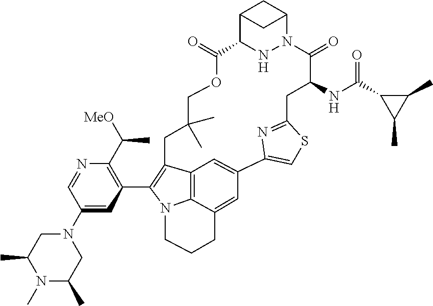

### Step 1: 3-(6-bromo-2-{2-[(1S)-1-methoxyethyl]-5-[(3R,5S)-3,4,5-trimethylpiperazin-1-yl]pyridin-3-yl}-1-azatricyclo[6.3.1.0^{4,12}]dodeca-2,4(12),5,7-tetraen-3-yl)-2,2-dimethylpropyl Acetate

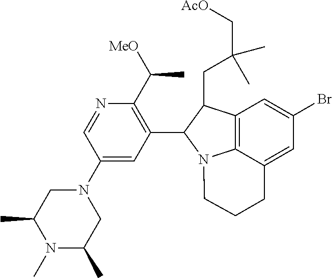 [SMILES: CO[C@@H](C)c1ncc(N2C[C@@H](C)N(C)[C@@H](C)C2)cc1C1C(CC(C)(C)COC(C)=O)c2cc(Br)cc3c2N1CCC3]

To a mixture of 3-(6-bromo-2-{2-[(1S)-1-methoxyethyl]-5-(4,4,5,5-tetramethyl-1,3,2-dioxaborolan-2-yl)pyridin-3-yl}-1-azatricyclo[6.3.1.0^{4,12}]dodeca-2,4(12),5,7-tetraen-3-yl)-2,2-dimethylpropyl acetate (8.0 g, 12.8 mmol) and (2S,6R)-1,2,6-trimethylpiperazine (8.19 g, 64 mmol) in MeCN (80 mL) was added TEA (3.88 g, 38.4 mmol) and Cu(OAc)₂ (4.66 g, 25.6 mmol). The mixture was stirred for 4 h at room temperature under oxygen atmosphere. The mixture was then diluted with EA (100 mL) and water (60 mL). The solid was filtered off and the filter cake was rinsed with EA (2×100 mL). The organic phase was separated and washed with water (100 mL), brine (100 mL), dried over Na₂SO₄, filtered and concentrated under vacuum. The residue was purified by silica gel column chromatography (DCM:MeOH=15:1) to give the title compound (4.2 g, 52%). LC-MS (M+H)⁺=625.3/627.3.

### Step 2: {3-[3-(acetyloxy)-2,2-dimethylpropyl]-2-{2-[(1S)-1-methoxyethyl]-5-[(3R,5S)-3,4,5-trimethylpiperazin-1-yl]pyridin-3-yl}-1-azatricyclo[6.3.1.0^{4,12}]dodeca-2,4(12),5,7-tetraen-6-yl}boronic Acid

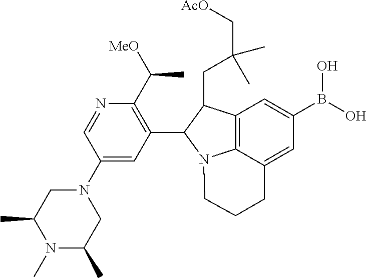 [SMILES: CO[C@@H](C)c1ncc(N2C[C@@H](C)N(C)[C@@H](C)C2)cc1C1C(CC(C)(C)COC(C)=O)c2cc(B(O)O)cc3c2N1CCC3]

To a mixture of 3-(6-bromo-2-{2-[(1S)-1-methoxyethyl]-5-[(3R,5S)-3,4,5-trimethylpiperazin-1-yl]pyridin-3-yl}-1-azatricyclo[6.3.1.0^{4,12}]dodeca-2,4(12),5,7-tetraen-3-yl)-2,2-dimethylpropyl acetate (4.2 g, 6.7 mmol) and B₂(OH)₄ (1.69 g, 18.7 mmol) in 2-MeTHF (30 mL) and MeOH (10 mL) was added KOPiv (2 g, 14.7 mmol), XPhos (0.639 g, 1.34 mmol), XPhos-Pd-G3 (566 mg, 0.67 mmol). The mixture was stirred for 2 h at 30° C. under nitrogen. The mixture was cooled to room temperature and then diluted with EA (100 mL). The mixture was washed with brine (20 mL). The organic phase was dried over Na₂SO₄, filtered and concentrated under vacuum. The residue was purified by prep-TLC (DCM:MeOH=15:1) to give the title compound (3.3 g, 84%). LC-MS (M+H)⁺=591.5.

### Step 3: methyl (4S)-2-[(2S)-3-(4-{3-[3-(acetyloxy)-2,2-dimethylpropyl]-2-{2-[(1S)-1-methoxyethyl]-5-[(3R,5S)-3,4,5-trimethylpiperazin-1-yl]pyridin-3-yl}-1-azatricyclo[6.3.1.0^{4,12}]dodeca-2,4(12),5,7-tetraen-6-yl}-1,3-thiazol-2-yl)-2-{[(tert-butoxy)carbonyl]amino}propanoyl]-2,3-diazabicyclo[3.1.1]heptane-4-carboxylate

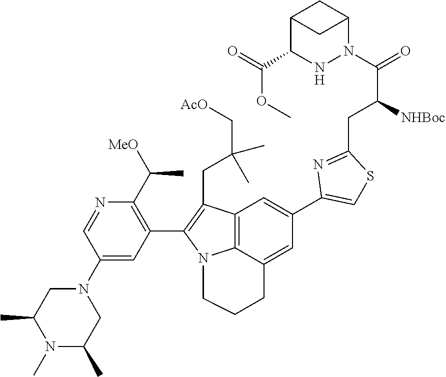 [SMILES: COC(=O)[C@H]1NN(C(=O)[C@H](Cc2nc(-c3cc4c5c(c3)c(CC(C)(C)COC(C)=O)c(-c3cc(N6C[C@@H](C)N(C)[C@@H](C)C6)cnc3[C@H](C)OC)n5CCC4)cs2)NC(=O)OC(C)(C)C)C2CC1C2]

To a mixture of {3-[3-(acetyloxy)-2,2-dimethylpropyl]-2-{2-[(1S)-1-methoxyethyl]-5-[(3R,5S)-3,4,5-trimethylpiperazin-1-yl]pyridin-3-yl}-1-azatricyclo[6.3.1.0^{4,12}]dodeca-2,4(12),5,7-tetraen-6-yl}boronic acid (3.3 g, 5.58 mmol) and methyl (4S)-2-[(2S)-3-(4-bromo-1,3-thiazol-2-yl)-2-{[(tert-butoxy)carbonyl]amino}propanoyl]-2,3-diazabicyclo[3.1.1]heptane-4-carboxylate (2.6 g, 5.31 mmol) in dioxane (100 mL) and water (10 mL) was added K₃PO₄ (3.38 g, 45.9 mmol) and Pd(dtbpf)Cl₂ (346 mg, 0.531 mmol). The solution was stirred for 3 h at 70° C. under nitrogen and then cooled to room temperature. The mixture was diluted with EA (100 mL), washed with water (50 mL), brine (50 mL). The organic phase was dried over Na₂SO₄, filtered and concentrated under vacuum. The residue was purified by silica gel column chromatography (DCM:MeOH=15:1) to give the title compound (2.8 g, 52%). LC-MS (M+H)⁺=955.5.

### Step 4: (4S)-2-[(2S)-2-{[(tert-butoxy)carbonyl]amino}-3-{4-[3-(3-hydroxy-2,2-dimethylpropyl)-2-{2-[(1S)-1-methoxyethyl]-5-[(3R,5S)-3,4,5-trimethylpiperazin-1-yl]pyridin-3-yl}-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic Acid

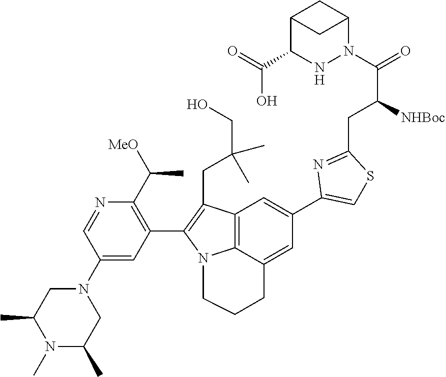 [SMILES: CO[C@@H](C)c1ncc(N2C[C@@H](C)N(C)[C@@H](C)C2)cc1-c1c(CC(C)(C)CO)c2cc(-c3csc(C[C@H](NC(=O)OC(C)(C)C)C(=O)N4N[C@H](C(=O)O)C5CC4C5)n3)cc3c2n1CCC3]

To a solution of methyl (4S)-2-[(2S)-3-(4-{3-[3-(acetyloxy)-2,2-dimethylpropyl]-2-{2-[(1S)-1-methoxyethyl]-5-[(3R,5S)-3,4,5-trimethylpiperazin-1-yl]pyridin-3-yl}-1-azatricyclo[6.3.1.0^{4,12}]dodeca-2,4(12),5,7-tetraen-6-yl}-1,3-thiazol-2-yl)-2-{[(tert-butoxy)carbonyl]amino}propanoyl]-2,3-diazabicyclo[3.1.1]heptane-4-carboxylate (2.8 g, 2.93 mmol) in THF (50 mL) and water (50 mL) was added LiOH·H₂O (616 mg, 14.6 mmol). The resulting mixture was stirred for 6 h at room temperature. The mixture was diluted with water (100 mL) and washed with EA (100 mL). The aqueous phase was acidified to pH ˜5 with 1 N hydrochloric acid at 0° C. The mixture was extracted with DCM/MeOH (10/1, 3×40 mL). The organic phases were combined, dried over Na₂SO₄, filtered and concentrated under vacuum to give the title compound (2.5 g, 95%). LC-MS (M+H)⁺=899.5.

### Step 5: tert-butyl N-[(7S,13S)-20-{2-[(1S)-1-methoxyethyl]-5-[(3R,5S)-3,4,5-trimethylpiperazin-1-yl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate

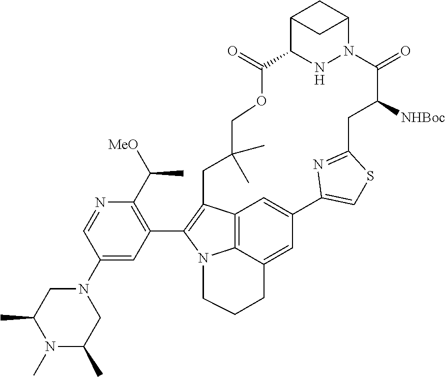 [SMILES: CO[C@@H](C)c1ncc(N2C[C@@H](C)N(C)[C@@H](C)C2)cc1-c1c2c3cc(cc4c3n1CCC4)-c1csc(n1)C[C@H](NC(=O)OC(C)(C)C)C(=O)N1N[C@H](C(=O)OCC(C)(C)C2)C2CC1C2]

To a solution of (4S)-2-[(2S)-2-{[(tert-butoxy)carbonyl]amino}-3-{4-[3-(3-hydroxy-2,2-dimethylpropyl)-2-{2-[(1S)-1-methoxyethyl]-5-[(3R,5S)-3,4,5-trimethylpiperazin-1-yl]pyridin-3-yl}-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic acid (2.5 g, 2.78 mmol) in MeCN (1250 mL) was added TCFH (2.34 g, 8.35 mmol) and NMI (1.37 g, 16.68 mmol) at room temperature. The mixture was stirred for 2 h at room temperature, and then the solvent was removed under vacuum. To this residue was added EA (100 mL) and the mixture was washed with water (100 mL) and brine (100 mL). The organic phase was dried over Na₂SO₄, filtered and concentrated under vacuum. The residue was purified by silica gel column chromatography (DCM:MeOH=15:1) to give the title compound (1.7 g, 69%). LC-MS (M+H)⁺=881.4.

### Step 6: (7S,13S)-7-amino-20-{2-[(1S)-1-methoxyethyl]-5-[(3R,5S)-3,4,5-trimethylpiperazin-1-yl]pyridin-3-yl}-17,17-dimethyl-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione

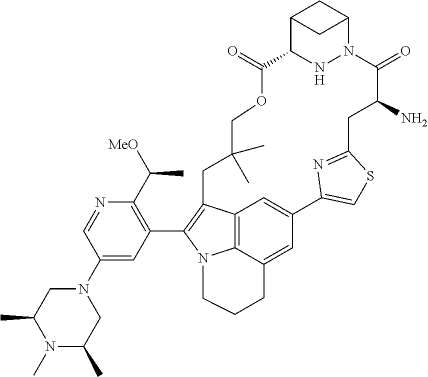 [SMILES: CO[C@@H](C)c1ncc(N2C[C@@H](C)N(C)[C@@H](C)C2)cc1-c1c2c3cc(cc4c3n1CCC4)-c1csc(n1)C[C@H](N)C(=O)N1N[C@H](C(=O)OCC(C)(C)C2)C2CC1C2]

A mixture of tert-butyl N-[(7S,13S)-20-{2-[(1S)-1-methoxyethyl]-5-[(3R,5S)-3,4,5-trimethylpiperazin-1-yl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate (1.7 g, 1.93 mmol) in HCl/dioxane (4 M, 30 mL) was stirred for 1 h at room temperature and then concentrated under vacuum. The residue was diluted with DCM (50 mL), washed with saturated NaHCO₃ (50 mL), dried over Na₂SO₄, filtered, and concentrated under vacuum to give the title compound (1.4 g, 93%). LC-MS (M+H)⁺=781.4

### Step 7: (1R,2R,3S)—N-[(7S,13S,19M)-20-{2-[(1S)-1-methoxyethyl]-5-[(3R,5S)-3,4,5-trimethylpiperazin-1-yl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

To a solution of (7S,13S)-7-amino-20-{2-[(1S)-1-methoxyethyl]-5-[(3R,5S)-3,4,5-trimethylpiperazin-1-yl]pyridin-3-yl}-17,17-dimethyl-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione (1.3 g, 1.66 mmol) in DMF (30 mL) was added DIEA (0.92 mL, 4.98 mmol), (1r,2R,3S)-2,3-dimethylcyclopropane-1-carboxylic acid (0.38 g, 3.32 mmol) and HATU (946 mg, 2.49 mmol). The mixture was stirred for 2 h at room temperature and then diluted with EA (40 mL), washed with brine (2×20 mL). The organic phase was dried over Na₂SO₄, filtered, and concentrated under vacuum. The residue was purified by prep-TLC (DCM:MeOH=10:1) to give a pair of atropisomers. The atropisomers were further separated by prep-HPLC to give example 20 (M-isomer, 280 mg, 19%). ¹H NMR (400 MHz, DMSO-d6) δ 8.57-8.32 (m, 2H), 8.20 (s, 1H), 7.67 (s, 1H), 7.39 (s, 1H), 7.12 (d, J=2.9 Hz, 1H), 5.88 (d, J=11.0 Hz, 1H), 5.34 (t, J=8.0 Hz, 1H), 4.62 (d, J=10.9 Hz, 1H), 4.53-4.38 (m, 1H), 4.33-4.04 (m, 2H), 3.76-3.64 (m, 2H), 3.62-3.43 (m, 3H), 3.24-3.13 (m, 4H), 3.08 (dd, J=14.6, 7.4 Hz, 1H), 3.01-2.76 (m, 3H), 2.66-2.57 (m, 1H), 3.30-3.20 (m, 4H), 2.31-2.00 (m, 9H), 1.50 (t, J=9.3 Hz, 1H), 1.30 (d, J=6.1 Hz, 3H), 1.22-1.09 (m, 3H), 1.08-0.97 (m, 12H), 0.84 (s, 3H), 0.35 (s, 3H). LC-MS (M+H)⁺=877.4.

### Example 21: (1S,2S)—N-[(7S,13S,19M)-20-{2-[(1S)-1-methoxyethyl]-5-[(3R,5S)-3,4,5-trimethylpiperazin-1-yl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2-methylcyclopropane-1-carboxamide

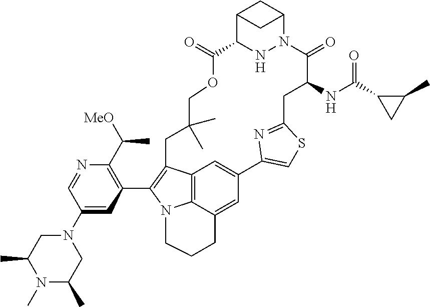

To a solution of (7S,13S)-7-amino-20-{2-[(1S)-1-methoxyethyl]-5-[(3R,5S)-3,4,5-trimethylpiperazin-1-yl]pyridin-3-yl}-17,17-dimethyl-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione (100 mg, 0.13 mmol) in DMF (4 mL) was added DIEA (49.5 mg, 0.384 mmol), (1S,2S)-2-methylcyclopropane-1-carboxylic acid (25.6 mg, 0.256 mmol) and HATU (73 mg, 0.192 mmol). The mixture was stirred for 2 h at room temperature and then diluted with EA (40 mL), washed with brine (2×10 mL). The organic phase was dried over Na₂SO₄, filtered, and concentrated under vacuum. The residue was purified by prep-TLC (DCM:MeOH=10:1) to give a pair of atropisomers. The atropisomers were further separated by prep-HPLC to give example 21 (M-isomer, 26 mg, 23%). ¹H NMR (400 MHz, DMSO-d6) δ 8.54 (d, J=8.9 Hz, 1H), 8.43 (d, J=2.9 Hz, 1H), 8.25 (s, 1H), 7.72 (s, 1H), 7.44 (s, 1H), 7.16 (d, J=3.0 Hz, 1H), 5.95 (d, J=11.0 Hz, 1H), 5.40 (t, J=8.3 Hz, 1H), 4.77-4.59 (m, 1H), 4.57-4.37 (m, 1H), 4.37-4.13 (m, 2H), 3.84-3.68 (m, 2H), 3.63-3.46 (m, 3H), 3.30-3.20 (m, 4H), 3.12 (dd, J=14.9, 7.3 Hz, 1H), 3.05-2.82 (m, 3H), 2.69-2.60 (m, 1H), 2.49-2.41 (m, 4H), 2.36-2.08 (m, 9H), 1.67-1.47 (m, 2H), 1.34 (d, J=6.0 Hz, 3H), 1.16-1.02 (m, 10H), 0.97-0.84 (m, 4H), 0.67-0.52 (m, 1H), 0.39 (s, 3H). LC-MS (M+H)⁺=863.4.

### Example 22: (1R,2R,3S)—N-[(7S,13S,19M)-20-{5-[(9aR)-octahydropyrazino[2,1-c][1,4]oxazin-8-yl]-2-[(1S)-1-methoxyethyl]pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

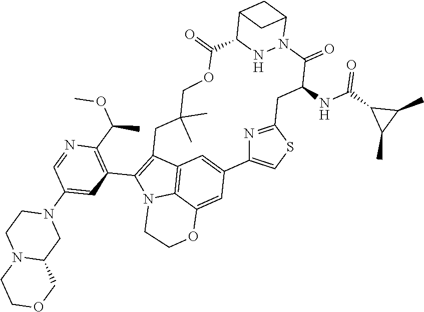

### Step 1: 3-(2-{5-[(9aR)-octahydropyrazino[2,1-c][1,4]oxazin-8-yl]-2-[(1S)-1-methoxyethyl]pyridin-3-yl}-6-bromo-9-oxa-1-azatricyclo[6.3.1.0^{4,12}]dodeca-2,4(12),5,7-tetraen-3-yl)-2,2-dimethylpropyl Acetate

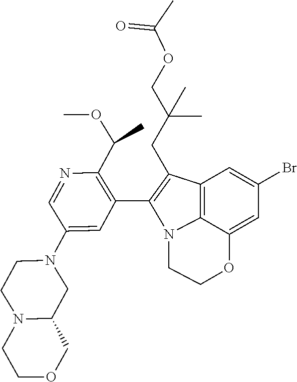 [SMILES: CO[C@@H](C)c1ncc(N2CCN3CCOC[C@H]3C2)cc1-c1c(CC(C)(C)COC(C)=O)c2cc(Br)cc3c2n1CCO3]

The title compound was prepared in a manner similar to that in example 9 step 2 from (5-{3-[3-(acetyloxy)-2,2-dimethylpropyl]-6-bromo-9-oxa-1-azatricyclo[6.3.1.0^{4,12}]dodeca-2,4(12),5,7-tetraen-2-yl}-6-[(1S)-1-methoxyethyl]pyridin-3-yl)boronic acid and (9aR)-octahydropyrazino[2,1-c][1,4]oxazine (550 mg, 47%). LC-MS (M+H)⁺=641.2/643.2.

### Step 2: 3-(2-{5-[(9aR)-octahydropyrazino[2,1-c][1,4]oxazin-8-yl]-2-[(1S)-1-methoxyethyl]pyridin-3-yl}-6-(4,4,5,5-tetramethyl-1,3,2-dioxaborolan-2-yl)-9-oxa-1-azatricyclo[6.3.1.0^{4,12}]dodeca-2,4(12),5,7-tetraen-3-yl)-2,2-dimethylpropyl Acetate

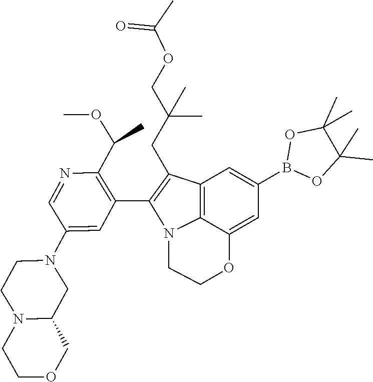 [SMILES: CO[C@@H](C)c1ncc(N2CCN3CCOC[C@H]3C2)cc1-c1c(CC(C)(C)COC(C)=O)c2cc(B3OC(C)(C)C(C)(C)O3)cc3c2n1CCO3]

The title compound was prepared in a manner similar to that in example 9 step 3 from 3-(2-{5-[(9aR)-octahydropyrazino[2,1-c][1,4]oxazin-8-yl]-2-[(1S)-1-methoxyethyl]pyridin-3-yl}-6-bromo-9-oxa-1-azatricyclo[6.3.1.0^{4,12}]dodeca-2,4(12),5,7-tetraen-3-yl)-2,2-dimethylpropyl acetate and BDP (423 mg, 74%). LC-MS (M+H)⁺=689.4.

### Step 3: methyl (4S)-2-[(2S)-3-[4-(2-{5-[(9aR)-octahydropyrazino[2,1-c][1,4]oxazin-8-yl]-2-[(1S)-1-methoxyethyl]pyridin-3-yl}-3-[3-(acetyloxy)-2,2-dimethylpropyl]-9-oxa-1-azatricyclo[6.3.1.0^{4,12}]dodeca-2,4(12),5,7-tetraen-6-yl)-1,3-thiazol-2-yl]-2-{[(tert-butoxy)carbonyl]amino}propanoyl]-2,3-diazabicyclo[3.1.1]heptane-4-carboxylate

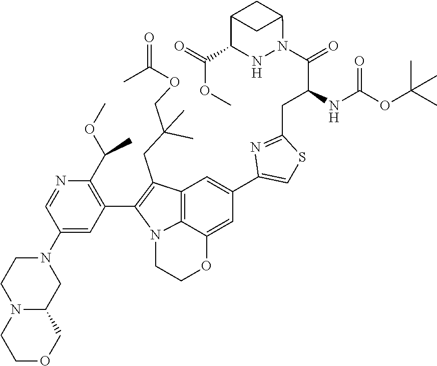 [SMILES: COC(=O)[C@H]1NN(C(=O)[C@H](Cc2nc(-c3cc4c5c(c3)c(CC(C)(C)COC(C)=O)c(-c3cc(N6CCN7CCOC[C@H]7C6)cnc3[C@H](C)OC)n5CCO4)cs2)NC(=O)OC(C)(C)C)C2CC1C2]

The title compound was prepared in a manner similar to that in example 9 step 4 from 3-(2-{5-[(9aR)-octahydropyrazino[2,1-c][1,4]oxazin-8-yl]-2-[(1S)-1-methoxyethyl]pyridin-3-yl}-6-(4,4,5,5-tetramethyl-1,3,2-dioxaborolan-2-yl)-9-oxa-1-azatricyclo[6.3.1.0^{4,12}]dodeca-2,4(12),5,7-tetraen-3-yl)-2,2-dimethylpropyl acetate and methyl (4S)-2-[(2S)-3-(4-bromo-1,3-thiazol-2-yl)-2-{[(tert-butoxy)carbonyl]amino}propanoyl]-2,3-diazabicyclo[3.1.1]heptane-4-carboxylate (458 mg, 77%). LC-MS (M+H)⁺=971.4.

### Step 4: (4S)-2-[(2S)-3-[4-(2-{5-[(9aR)-octahydropyrazino[2,1-c][1,4]oxazin-8-yl]-2-[(1S)-1-methoxyethyl]pyridin-3-yl}-3-(3-hydroxy-2,2-dimethylpropyl)-9-oxa-1-azatricyclo[6.3.1.0^{4,12}]dodeca-2,4(12),5,7-tetraen-6-yl)-1,3-thiazol-2-yl]-2-{[(tert-butoxy)carbonyl]amino}propanoyl]-2,3-diazabicyclo[3.1.1]heptane-4-carboxylic Acid

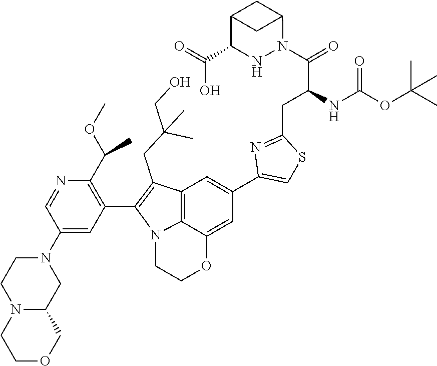 [SMILES: CO[C@@H](C)c1ncc(N2CCN3CCOC[C@H]3C2)cc1-c1c(CC(C)(C)CO)c2cc(-c3csc(C[C@H](NC(=O)OC(C)(C)C)C(=O)N4N[C@H](C(=O)O)C5CC4C5)n3)cc3c2n1CCO3]

The title compound was prepared in a manner similar to that in example 9 step 5 from methyl (4S)-2-[(2S)-3-[4-(2-{5-[(9aR)-octahydropyrazino[2,1-c][1,4]oxazin-8-yl]-2-[(1S)-1-methoxyethyl]pyridin-3-yl}-3-[3-(acetyloxy)-2,2-dimethylpropyl]-9-oxa-1-azatricyclo[6.3.1.0^{4,12}]dodeca-2,4(12),5,7-tetraen-6-yl)-1,3-thiazol-2-yl]-2-{[(tert-butoxy)carbonyl]amino}propanoyl]-2,3-diazabicyclo[3.1.1]heptane-4-carboxylate (360 mg, 84%). LC-MS (M+H)⁺=915.4.

### Step 5: tert-butyl N-[(7S,13S)-20-{5-[(9aR)-octahydropyrazino[2,1-c][1,4]oxazin-8-yl]-2-[(1S)-1-methoxyethyl]pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate

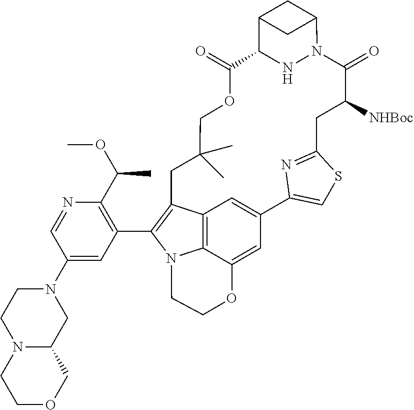 [SMILES: CO[C@@H](C)c1ncc(N2CCN3CCOC[C@H]3C2)cc1-c1c2c3cc(cc4c3n1CCO4)-c1csc(n1)C[C@H](NC(=O)OC(C)(C)C)C(=O)N1N[C@H](C(=O)OCC(C)(C)C2)C2CC1C2]

The title compound was prepared in a manner similar to that in example 9 step 6 from (4S)-2-[(2S)-3-[4-(2-{5-[(9aR)-octahydropyrazino[2,1-c][1,4]oxazin-8-yl]-2-[(1S)-1-methoxyethyl]pyridin-3-yl}-3-(3-hydroxy-2,2-dimethylpropyl)-9-oxa-1-azatricyclo[6.3.1.0^{4,12}]dodeca-2,4(12),5,7-tetraen-6-yl)-1,3-thiazol-2-yl]-2-{[(tert-butoxy)carbonyl]amino}propanoyl]-2,3-diazabicyclo[3.1.1]heptane-4-carboxylic acid (65 mg, 36%). LC-MS (M+H)⁺=897.4.

### Step 6: (7S,13S)-20-{5-[(9aR)-octahydropyrazino[2,1-c][1,4]oxazin-8-yl]-2-[(1S)-1-methoxyethyl]pyridin-3-yl}-7-amino-17,17-dimethyl-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione

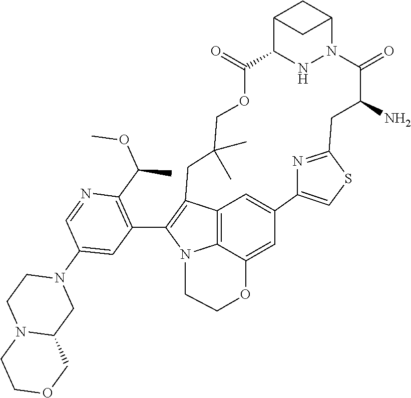 [SMILES: CO[C@@H](C)c1ncc(N2CCN3CCOC[C@H]3C2)cc1-c1c2c3cc(cc4c3n1CCO4)-c1csc(n1)C[C@H](N)C(=O)N1N[C@H](C(=O)OCC(C)(C)C2)C2CC1C2]

The title compound was prepared in a manner similar to that in example 9 step 7 from tert-butyl N-[(7S,13S)-20-{5-[(9aR)-octahydropyrazino[2,1-c][1,4]oxazin-8-yl]-2-[(1S)-1-methoxyethyl]pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate (60 mg, 100%). LC-MS (M+H)⁺=797.4.

### Step 7: (1R,2R,3S)—N-[(7S,13S,19M)-20-{5-[(9aR)-octahydropyrazino[2,1-c][1,4]oxazin-8-yl]-2-[(1S)-1-methoxyethyl]pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

Example 22 (10 mg, 15%) was prepared in a manner similar to that in example 9 step 8 from (7S,13S)-20-{5-[(9aR)-octahydropyrazino[2,1-c][1,4]oxazin-8-yl]-2-[(1S)-1-methoxyethyl]pyridin-3-yl}-7-amino-17,17-dimethyl-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione and (1r,2R,3S)-2,3-dimethylcyclopropane-1-carboxylic acid. LC-MS (M+H)⁺=893.4. ¹H NMR (400 MHz, DMSO-d6) δ 8.41 (d, J=9.3 Hz, 2H), 8.01 (s, 1H), 7.75 (s, 1H), 7.18 (d, J=2.4 Hz, 1H), 7.13 (s, 1H), 5.89 (d, J=11.0 Hz, 1H), 5.30 (t, J=8.1 Hz, 1H), 4.68 (d, J=11.0 Hz, 1H), 4.58-4.54 (m, 2H), 4.46 (d, J=4.9 Hz, 1H), 4.28 (t, J=9.1 Hz, 2H), 3.79-3.16 (m, 7H), 3.19 (s, 4H), 3.09 (t, J=10.2 Hz, 2H), 2.91-2.77 (m, 3H), 2.68-2.61 (m, 3H), 2.31-2.13 (m, 6H), 1.56 (t, J=9.0 Hz, 1H), 1.31 (d, J=6.0 Hz, 3H), 1.24-1.16 (m, 2H), 1.13 (d, J=4.0 Hz, 2H), 1.04 (dd, J=11.1, 5.7 Hz, 6H), 0.90 (s, 3H), 0.34 (s, 3H).

### Example 23: (1R,2R,3S)—N-[(7S,13S,19M)-20-{5-[(9aS)-octahydropyrazino[2,1-c][1,4]oxazin-8-yl]-2-[(1S)-1-methoxyethyl]pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

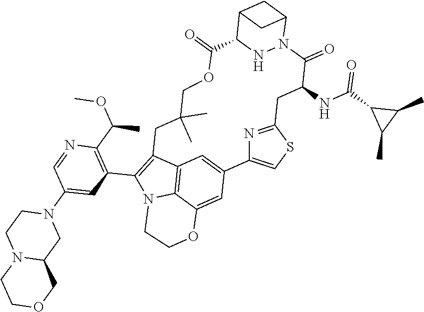

### Step 1: 3-(2-{5-[(9aS)-octahydropyrazino[2,1-c][1,4]oxazin-8-yl]-2-[(1S)-1-methoxyethyl]pyridin-3-yl}-6-bromo-9-exa-1-azatricyclo[6.3.1.0^{4,12}]dodeca-2,4(12),5,7-tetraen-3-yl)-2,2-dimethylpropyl Acetate

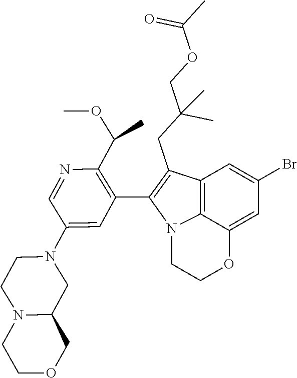 [SMILES: CO[C@@H](C)c1ncc(N2CCN3CCOC[C@@H]3C2)cc1-c1c(CC(C)(C)COC(C)=O)c2cc(Br)cc3c2n1CCO3]

The title compound (343 mg, 49%) was prepared in a manner similar to that in example 9 step 2 from (5-{3-[3-(acetyloxy)-2,2-dimethylpropyl]-6-bromo-9-oxa-1-azatricyclo[6.3.1.0^{4,12}]dodeca-2,4(12),5,7-tetraen-2-yl}-6-[(1S)-1-methoxyethyl]pyridin-3-yl)boronic acid and (9aS)-octahydropyrazino[2,1-c][1,4]oxazine. LC-MS (M+H)⁺=641.2/643.2.

### Step 2: 3-(2-{5-[(9aS)-octahydropyrazino[2,1-c][1,4]oxazin-8-yl]-2-[(1S)-1-methoxyethyl]pyridin-3-yl}-6-(4,4,5,5-tetramethyl-1,3,2-dioxaborolan-2-yl)-9-oxa-1-azatricyclo[6.3.1.0^{4,12}]dodeca-2,4(12),5,7-tetraen-3-yl)-2,2-dimethylpropyl Acetate

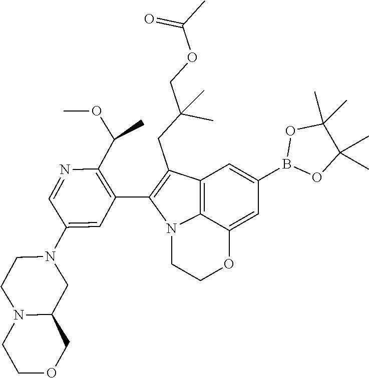 [SMILES: CO[C@@H](C)c1ncc(N2CCN3CCOC[C@@H]3C2)cc1-c1c(CC(C)(C)COC(C)=O)c2cc(B3OC(C)(C)C(C)(C)O3)cc3c2n1CCO3]

The title compound (323 mg, 88%) was prepared in a manner similar to that in example 9 step 3 from 3-(2-{5-[(9aS)-octahydropyrazino[2,1-c][1,4]oxazin-8-yl]-2-[(1S)-1-methoxyethyl]pyridin-3-yl}-6-bromo-9-oxa-1-azatricyclo[6.3.1.0^{4,12}]dodeca-2,4(12),5,7-tetraen-3-yl)-2,2-dimethylpropyl acetate. LC-MS (M+H)⁺=689.4.

### Step 3: methyl (4S)-2-[(2S)-3-[4-(2-{5-[(9aS)-octahydropyrazino[2,1-c][1,4]oxazin-8-yl]-2-[(1S)-1-methoxyethyl]pyridin-3-yl}-3-[3-(acetyloxy)-2,2-dimethylpropyl]-9-oxa-1-azatricyclo[6.3.1.0^{4,12}]dodeca-2,4(12),5,7-tetraen-6-yl)-1,3-thiazol-2-yl]-2-{[(tert-butoxy)carbonyl]amino}propanoyl]-2,3-diazabicyclo[3.1.1]heptane-4-carboxylate

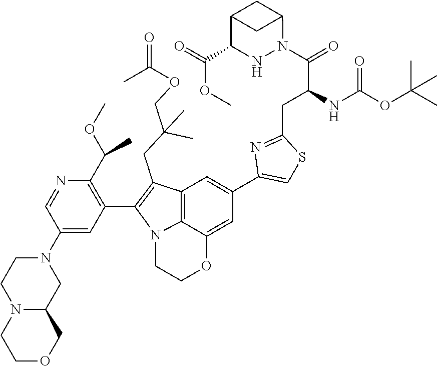 [SMILES: COC(=O)[C@H]1NN(C(=O)[C@H](Cc2nc(-c3cc4c5c(c3)c(CC(C)(C)COC(C)=O)c(-c3cc(N6CCN7CCOC[C@@H]7C6)cnc3[C@H](C)OC)n5CCO4)cs2)NC(=O)OC(C)(C)C)C2CC1C2]

The title compound (290 mg, 64%) was prepared in a manner similar to that in example 9 step 4 from 3-(2-{5-[(9aS)-octahydropyrazino[2,1-c][1,4]oxazin-8-yl]-2-[(1S)-1-methoxyethyl]pyridin-3-yl}-6-(4,4,5,5-tetramethyl-1,3,2-dioxaborolan-2-yl)-9-oxa-1-azatricyclo[6.3.1.0^{4,12}]dodeca-2,4(12),5,7-tetraen-3-yl)-2,2-dimethylpropyl acetate and methyl (4S)-2-[(2S)-3-(4-bromo-1,3-thiazol-2-yl)-2-{[(tert-butoxy)carbonyl]amino}propanoyl]-2,3-diazabicyclo[3.1.1]heptane-4-carboxylate. LC-MS (M+H)⁺=971.4.

### Step 4: (4S)-2-[(2S)-3-[4-(2-{5-[(9aS)-octahydropyrazino[2,1-c][1,4]oxazin-8-yl]-2-[(1S)-1-methoxyethyl]pyridin-3-yl}-3-(3-hydroxy-2,2-dimethylpropyl)-9-oxa-1-azatricyclo[6.3.1.0^{4,12}]dodeca-2,4(12),5,7-tetraen-6-yl)-1,3-thiazol-2-yl]-2-{[(tert-butoxy)carbonyl]amino}propanoyl]-2,3-diazabicyclo[3.1.1]heptane-4-carboxylic Acid

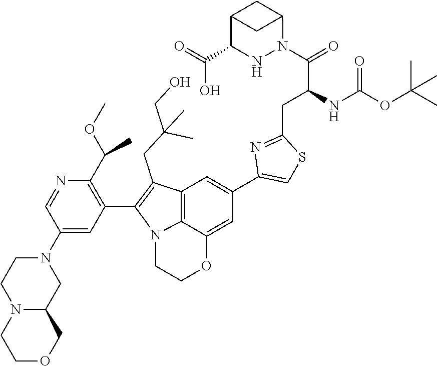 [SMILES: CO[C@@H](C)c1ncc(N2CCN3CCOC[C@@H]3C2)cc1-c1c(CC(C)(C)CO)c2cc(-c3csc(C[C@H](NC(=O)OC(C)(C)C)C(=O)N4N[C@H](C(=O)O)C5CC4C5)n3)cc3c2n1CCO3]

The title compound (190 mg, 70%) was prepared in a manner similar to that in example 9 step 5 from methyl (4S)-2-[(2S)-3-[4-(2-{5-[(9aS)-octahydropyrazino[2,1-c][1,4]oxazin-8-yl]-2-[(1S)-1-methoxyethyl]pyridin-3-yl}-3-[3-(acetyloxy)-2,2-dimethylpropyl]-9-oxa-1-azatricyclo[6.3.1.0⁴ 2]dodeca-2,4(12),5,7-tetraen-6-yl)-1,3-thiazol-2-yl]-2-{[(tert-butoxy)carbonyl]amino}propanoyl]-2,3-diazabicyclo[3.1.1]heptane-4-carboxylate. LC-MS (M+H)⁺=915.4.

### Step 5: tert-butyl N-[(7S,13S)-20-{5-[(9aS)-octahydropyrazino[2,1-c][1,4]oxazin-8-yl]-2-[(15)-1-methoxyethyl]pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate

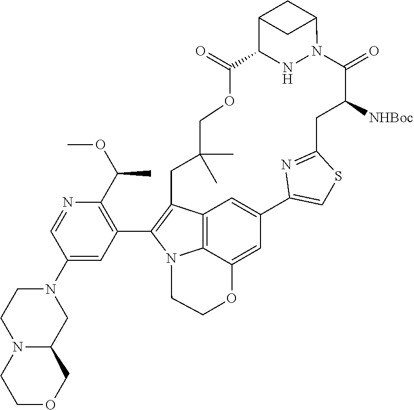 [SMILES: CO[C@@H](C)c1ncc(N2CCN3CCOC[C@@H]3C2)cc1-c1c2c3cc(cc4c3n1CCO4)-c1csc(n1)C[C@H](NC(=O)OC(C)(C)C)C(=O)N1N[C@H](C(=O)OCC(C)(C)C2)C2CC1C2]

The title compound (46 mg, 24%) was prepared in a manner similar to that in example 9 step 6 from (4S)-2-[(2S)-3-[4-(2-{5-[(9aS)-octahydropyrazino[2,1-c][1,4]oxazin-8-yl]-2-[(1S)-1-methoxyethyl]pyridin-3-yl}-3-(3-hydroxy-2,2-dimethylpropyl)-9-oxa-1-azatricyclo[6.3.1.0^{4,12}]dodeca-2,4(12),5,7-tetraen-6-yl)-1,3-thiazol-2-yl]-2-{[(tert-butoxy)carbonyl]amino}propanoyl]-2,3-diazabicyclo[3.1.1]heptane-4-carboxylic acid. LC-MS (M+H)⁺=897.4.

### Step 6: (7S,13S)-20-{5-[(9aS)-octahydropyrazino[2,1-c][1,4]oxazin-8-yl]-2-[(1S)-1-methoxyethyl]pyridin-3-yl}-7-amino-17,17-dimethyl-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione

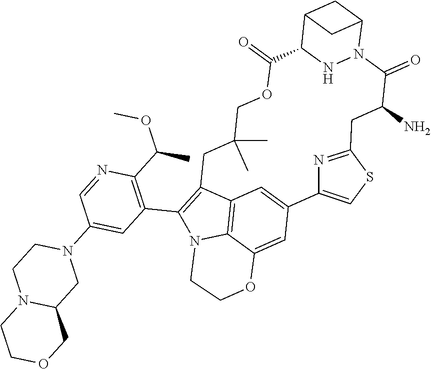 [SMILES: CO[C@@H](C)c1ncc(N2CCN3CCOC[C@@H]3C2)cc1-c1c2c3cc(cc4c3n1CCO4)-c1csc(n1)C[C@H](N)C(=O)N1N[C@H](C(=O)OCC(C)(C)C2)C2CC1C2]

The title compound (48 mg, 100%) was prepared in a manner similar to that in example 9 step 7 from tert-butyl N-[(7S,13S)-20-{5-[(9aS)-octahydropyrazino[2,1-c][1,4]oxazin-8-yl]-2-[(1S)-1-methoxyethyl]pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate. LC-MS (M+H)⁺=797.4.

### Step 7: (1R,2R,3S)—N-[(7S,13S,19M)-20-{5-[(9aS)-octahydropyrazino[2,1-c][1,4]oxazin-8-yl]-2-[(1S)-1-methoxyethyl]pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

Example 23 was prepared in a manner similar to that in example 9 step 8 from (7S,13S)-20-{5-[(9aS)-octahydropyrazino[2,1-c][1,4]oxazin-8-yl]-2-[(1S)-1-methoxyethyl]pyridin-3-yl}-7-amino-17,17-dimethyl-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione and (1r,2R,3S)-2,3-dimethylcyclopropane-1-carboxylic acid (12 mg, 27%). ¹H NMR (400 MHz, DMSO-d6) δ 8.41 (d, J=9.3 Hz, 2H), 8.01 (s, 1H), 7.75 (s, 1H), 7.18 (d, J=2.4 Hz, 1H), 7.13 (s, 1H), 5.89 (d, J=11.0 Hz, 1H), 5.30 (t, J=8.1 Hz, 1H), 4.68 (d, J=11.0 Hz, 1H), 4.58-4.54 (m, 2H), 4.46 (d, J=4.9 Hz, 1H), 4.28 (t, J=9.1 Hz, 2H), 3.79-3.16 (m, 7H), 3.19 (s, 4H), 3.09 (t, J=10.2 Hz, 2H), 2.91-2.77 (m, 3H), 2.68-2.61 (m, 3H), 2.31-2.13 (m, 6H), 1.56 (t, J=9.0 Hz, 1H), 1.31 (d, J=6.0 Hz, 3H), 1.24-1.16 (m, 2H), 1.13 (d, J=4.0 Hz, 2H), 1.04 (dd, J=11.1, 5.7 Hz, 6H), 0.90 (s, 3H), 0.34 (s, 3H). LC-MS (M+H)⁺=893.4.

Atrop-24 & Example 24: (1R,2R,3S)—N-[(7S,13S,19P)-20-[5-(4-ethylpiperazin-1-yl)-2-[(1S)-1-methoxyethyl]pyridin-3-yl]-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide and (1R,2R,3S)—N-[(7S,13S,19M)-20-[5-(4-ethylpiperazin-1-yl)-2-[(1S)-1-methoxyethyl]pyridin-3-yl]-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

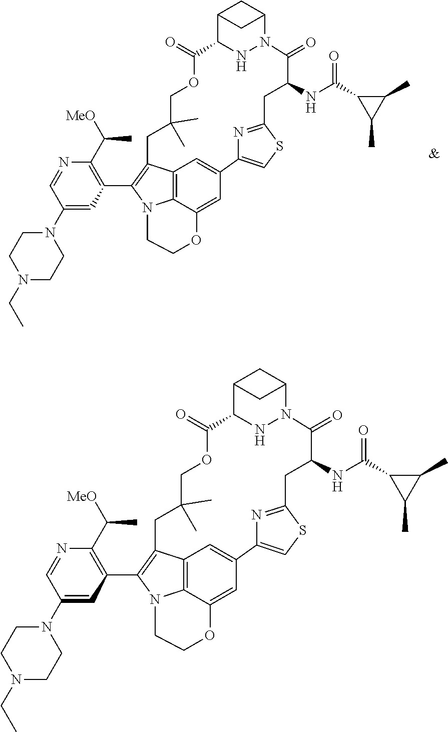

### Step 1: 3-{6-bromo-2-[5-(4-ethylpiperazin-1-yl)-2-[(1S)-1-methoxyethyl]pyridin-3-yl]-9-oxa-1-azatricyclo[6.3.1.0^{4,12}]dodeca-2,4(12),5,7-tetraen-3-yl}-2,2-dimethylpropyl Acetate

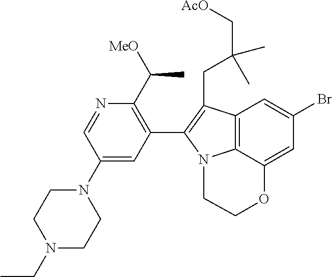 [SMILES: CCN1CCN(c2cnc([C@H](C)OC)c(-c3c(CC(C)(C)COC(C)=O)c4cc(Br)cc5c4n3CCO5)c2)CC1]

The title compound (250 mg, 62%) was prepared in a manner similar to that in example 7 step 2 from 3-(6-bromo-2-{2-[(1S)-1-methoxyethyl]-5-(4,4,5,5-tetramethyl-1,3,2-dioxaborolan-2-yl)pyridin-3-yl}-9-oxa-1-azatricyclo[6.3.1.0^{4,12}]dodeca-2,4(12),5,7-tetraen-3-yl)-2,2-dimethylpropyl acetate and 1-ethylpiperazine. LC-MS (M+H)⁺=613.3/615.3.

### Step 2: 3-{2-[5-(4-ethylpiperazin-1-yl)-2-[(1S)-1-methoxyethyl]pyridin-3-yl]-6-(4,4,5,5-tetramethyl-1,3,2-dioxaborolan-2-yl)-9-oxa-1-azatricyclo[6.3.1.0^{4,12}]dodeca-2,4(12),5,7-tetraen-3-yl}-2,2-dimethylpropyl Acetate

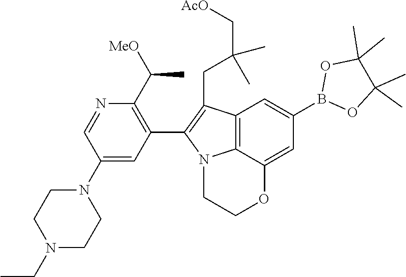 [SMILES: CCN1CCN(c2cnc([C@H](C)OC)c(-c3c(CC(C)(C)COC(C)=O)c4cc(B5OC(C)(C)C(C)(C)O5)cc5c4n3CCO5)c2)CC1]

The title compound (180 mg, 67%) was prepared in a manner similar to that in example 7 step 3 from 3-{6-bromo-2-[5-(4-ethylpiperazin-1-yl)-2-[(1S)-1-methoxyethyl]pyridin-3-yl]-9-oxa-1-azatricyclo[6.3.1.0^{4,12}]dodeca-2,4(12),5,7-tetraen-3-yl}-2,2-dimethylpropyl acetate. LC-MS (M+H)⁺=661.5.

### Step 3: methyl (4S)-2-[(2S)-3-(4-{3-[3-(acetyloxy)-2,2-dimethylpropyl]-2-[5-(4-ethylpiperazin-1-yl)-2-[(1S)-1-methoxyethyl]pyridin-3-yl]-9-oxa-1-azatricyclo[6.3.1.0^{4,12}]dodeca-2,4(12),5,7-tetraen-6-yl}-1,3-thiazol-2-yl)-2-{[(tert-butoxy)carbonyl]amino}propanoyl]-2,3-diazabicyclo[3.1.1]heptane-4-carboxylate

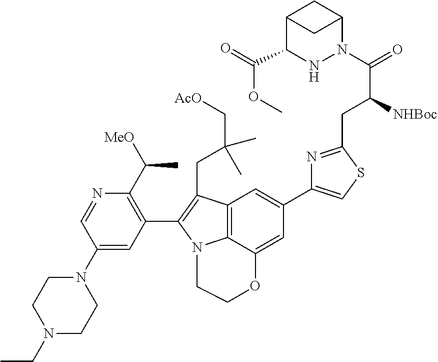 [SMILES: CCN1CCN(c2cnc([C@H](C)OC)c(-c3c(CC(C)(C)COC(C)=O)c4cc(-c5csc(C[C@H](NC(=O)OC(C)(C)C)C(=O)N6N[C@H](C(=O)OC)C7CC6C7)n5)cc5c4n3CCO5)c2)CC1]

The title compound (145 mg, 56%) was prepared in a manner similar to that in example 7 step 4 from 3-{2-[5-(4-ethylpiperazin-1-yl)-2-[(1S)-1-methoxyethyl]pyridin-3-yl]-6-(4,4,5,5-tetramethyl-1,3,2-dioxaborolan-2-yl)-9-oxa-1-azatricyclo[6.3.1.0^{4,12}]dodeca-2,4(12),5,7-tetraen-3-yl}-2,2-dimethylpropyl acetate and methyl (4S)-2-[(2S)-3-(4-bromo-1,3-thiazol-2-yl)-2-{[(tert-butoxy)carbonyl]amino}propanoyl]-2,3-diazabicyclo[3.1.1]heptane-4-carboxylate. LC-MS (M+H)⁺=943.3.

### Step 4: (4S)-2-[(2S)-2-{[(tert-butoxy)carbonyl]amino}-3-(4-{2-[5-(4-ethylpiperazin-1-yl)-2-[(1S)-1-methoxyethyl]pyridin-3-yl]-3-(3-hydroxy-2,2-dimethylpropyl)-9-oxa-1-azatricyclo[6.3.1.0^{4,12}]dodeca-2,4(12),5,7-tetraen-6-yl}-1,3-thiazol-2-yl)propanoyl]-2,3-diazabicyclo[3.1.1]heptane-4-carboxylic Acid

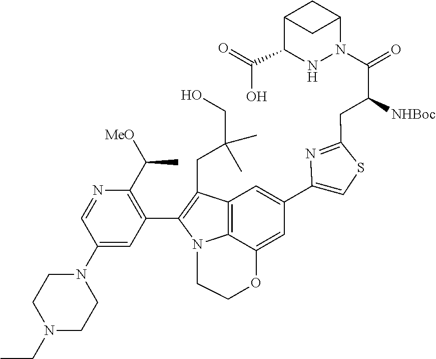 [SMILES: CCN1CCN(c2cnc([C@H](C)OC)c(-c3c(CC(C)(C)CO)c4cc(-c5csc(C[C@H](NC(=O)OC(C)(C)C)C(=O)N6N[C@H](C(=O)O)C7CC6C7)n5)cc5c4n3CCO5)c2)CC1]

The title compound (130 mg, 95%) was prepared in a manner similar to that in example 7 step 5 from methyl (4S)-2-[(2S)-3-(4-{3-[3-(acetyloxy)-2,2-dimethylpropyl]-2-[5-(4-ethylpiperazin-1-yl)-2-[(1S)-1-methoxyethyl]pyridin-3-yl]-9-oxa-1-azatricyclo[6.3.1.0^{4,12}]dodeca-2,4(12),5,7-tetraen-6-yl}-1,3-thiazol-2-yl)-2-{[(tert-butoxy)carbonyl]amino}propanoyl]-2,3-diazabicyclo[3.1.1]heptane-4-carboxylate. LC-MS (M+H)⁺=887.3.

### Step 5: tert-butyl N-[(7S,13S)-20-[5-(4-ethylpiperazin-1-yl)-2-[(1S)-1-methoxyethyl]pyridin-3-yl]-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate

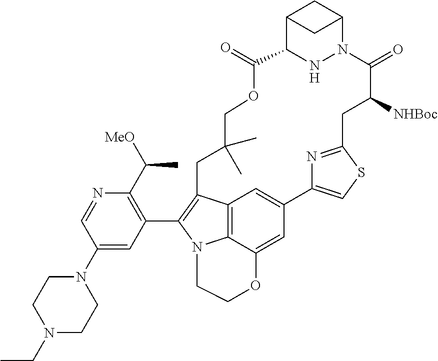 [SMILES: CCN1CCN(c2cnc([C@H](C)OC)c(-c3c4c5cc(cc6c5n3CCO6)-c3csc(n3)C[C@H](NC(=O)OC(C)(C)C)C(=O)N3N[C@H](C(=O)OCC(C)(C)C4)C4CC3C4)c2)CC1]

The title compound (40 mg, 32%) was prepared in a manner similar to that in example 7 step 6 from (4S)-2-[(2S)-2-{[(tert-butoxy)carbonyl]amino}-3-(4-{2-[5-(4-ethylpiperazin-1-yl)-2-[(1S)-1-methoxyethyl]pyridin-3-yl]-3-(3-hydroxy-2,2-dimethylpropyl)-9-oxa-1-azatricyclo[6.3.1.0^{4,12}]dodeca-2,4(12),5,7-tetraen-6-yl}-1,3-thiazol-2-yl)propanoyl]-2,3-diazabicyclo[3.1.1]heptane-4-carboxylic acid. LC-MS (M+H)⁺=869.5.

### Step 6: (7S,13S)-7-amino-20-[5-(4-ethylpiperazin-1-yl)-2-[(1S)-1-methoxyethyl]pyridin-3-yl]-17,17-dimethyl-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione

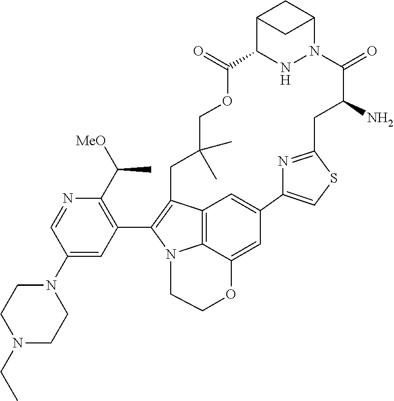 [SMILES: CCN1CCN(c2cnc([C@H](C)OC)c(-c3c4c5cc(cc6c5n3CCO6)-c3csc(n3)C[C@H](N)C(=O)N3N[C@H](C(=O)OCC(C)(C)C4)C4CC3C4)c2)CC1]

The title compound (30 mg, 85%) was prepared in a manner similar to that in example 9 step 7 from tert-butyl N-[(7S,13S)-20-[5-(4-ethylpiperazin-1-yl)-2-[(1S)-1-methoxyethyl]pyridin-3-yl]-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate. LC-MS (M+H)⁺=769.5.

### Step 7: (1R,2R,3S)—N-[(7S,13S,19P)-20-[5-(4-ethylpiperazin-1-yl)-2-[(1S)-1-methoxyethyl]pyridin-3-yl]-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide and (1R,2R,3S)—N-[(7S,13S,19M)-20-[5-(4-ethylpiperazin-1-yl)-2-[(1S)-1-methoxyethyl]pyridin-3-yl]-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

Atrop-24 (P-isomer, 4.0 mg, 12%) and example 24 (M-isomer, 8.0 mg, 24%) were prepared in a manner similar to that in example 9 step 8 from (7S,13S)-7-amino-20-[5-(4-ethylpiperazin-1-yl)-2-[(1S)-1-methoxyethyl]pyridin-3-yl]-17,17-dimethyl-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione and (1r,2R,3S)-2,3-dimethylcyclopropane-1-carboxylic acid.

Atrop-24: ¹H NMR (400 MHz, DMSO-d6) δ 8.40 (d, J=8.0 Hz, 2H), 8.04 (s, 1H), 7.75 (s, 1H), 7.36 (s, 1H), 7.13 (s, 1H), 5.85 (d, J=12.0 Hz, 1H), 5.32-5.24 (m, 1H), 4.71 (d, J=12.0 Hz, 1H), 4.48 (s, 2H), 4.41 (s, 1H), 4.05-3.97 (m, 2H), 3.68-3.46 (m, 3H), 3.24-3.20 (m, 3H), 3.19-3.16 (m, 2H), 3.14-3.10 (m, 1H), 3.08 (s, 3H), 2.99-2.94 (m, 1H), 2.66-2.62 (m, 1H), 2.40-2.35 (m, 2H), 2.34-2.31 (m, 2H), 2.31-2.29 (m, 1H), 2.14-2.08 (m, 1H), 2.00-1.93 (m, 1H), 1.63-1.56 (m, 1H), 1.22-1.08 (m, 3H), 1.15-1.10 (m, 5H), 1.08-1.02 (m, 6H), 1.01-0.96 (m, 4H), 0.91 (s, 3H), 0.47 (s, 3H). LC-MS (M+H)⁺=865.1.

Example 24: ¹H NMR (400 MHz, DMSO-d6) δ 8.42 (d, J=8.0 Hz, 2H), 8.01 (s, 1H), 7.76 (s, 1H), 7.17-7.11 (m, 2H), 5.89 (d, J=12.0 Hz, 1H), 5.34-5.26 (m, 1H), 4.68 (d, J=12.0 Hz, 1H), 4.63-4.53 (m, 2H), 4.48-4.44 (m, 1H), 4.31-4.25 (m, 2H), 3.72-3.67 (m, 1H), 3.57-3.48 (m, 3H), 3.42-3.39 (m, 1H), 3.24-3.21 (m, 4H), 3.20 (s, 3H), 3.11-3.05 (m, 1H), 2.91-2.86 (m, 1H), 2.65-2.61 (m, 2H), 2.34-2.29 (m, 3H), 2.14-2.06 (m, 1H), 1.60-1.54 (m, 1H), 1.30 (d, J=6.0 Hz, 3H), 1.23-1.19 (m, 3H), 1.15-1.11 (m, 2H), 1.07-1.02 (m, 6H), 1.01-0.96 (m, 4H), 0.90 (s, 3H), 0.83-0.76 (m, 1H), 0.33 (s, 3H). LC-MS (M+H)⁺=865.1.

Atrop-25 & Example 25: (1R,2R,3S)—N-[(7S,13S,19P)-20-{2-[(1S)-1-methoxyethyl]-5-[4-(propan-2-yl)piperazin-1-yl]pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide and (1R,2R,3S)—N-[(7S,13S,19M)-20-{2-[(1S)-1-methoxyethyl]-5-[4-(propan-2-yl)piperazin-1-yl]pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

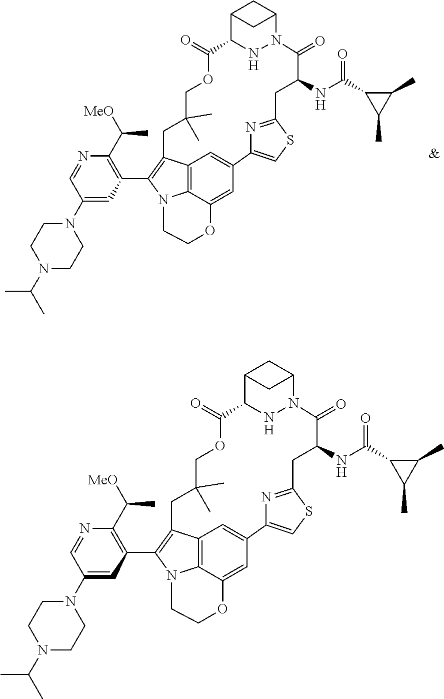

### Step 1: 3-(6-bromo-2-{2-[(1S)-1-methoxyethyl]-5-[4-(propan-2-yl)piperazin-1-yl]pyridin-3-yl}-9-oxa-1-azatricyclo[6.3.1.0^{4,12}]dodeca-2,4(12),5,7-tetraen-3-yl)-2,2-dimethylpropyl Acetate

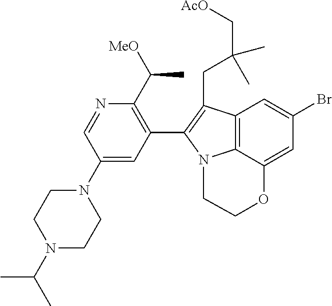 [SMILES: CO[C@@H](C)c1ncc(N2CCN(C(C)C)CC2)cc1-c1c(CC(C)(C)COC(C)=O)c2cc(Br)cc3c2n1CCO3]

The title compound (270 mg, 61%) was prepared in a manner similar to that in example 7 step 2 from 3-(6-bromo-2-{2-[(1S)-1-methoxyethyl]-5-(4,4,5,5-tetramethyl-1,3,2-dioxaborolan-2-yl)pyridin-3-yl}-9-oxa-1-azatricyclo[6.3.1.0^{4,12}]dodeca-2,4(12),5,7-tetraen-3-yl)-2,2-dimethylpropyl acetate and 1-(propan-2-yl)piperazine. LC-MS (M+H)⁺=627.3/629.3.

### Step 2: 3-(2-{2-[(1S)-1-methoxyethyl]-5-[4-(propan-2-yl)piperazin-1-yl]pyridin-3-yl}-6-(4,4,5,5-tetramethyl-1,3,2-dioxaborolan-2-yl)-9-oxa-1-azatricyclo[6.3.1.0^{4,12}]dodeca-2,4(12),5,7-tetraen-3-yl)-2,2-dimethylpropyl Acetate

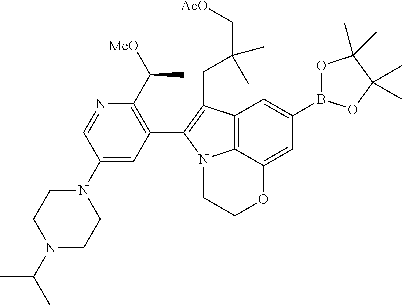 [SMILES: CO[C@@H](C)c1ncc(N2CCN(C(C)C)CC2)cc1-c1c(CC(C)(C)COC(C)=O)c2cc(B3OC(C)(C)C(C)(C)O3)cc3c2n1CCO3]

The title compound (200 mg, 69%) was prepared in a manner similar to that in example 7 step 3 from 3-(6-bromo-2-{2-[(1S)-1-methoxyethyl]-5-[4-(propan-2-yl)piperazin-1-yl]pyridin-3-yl}-9-oxa-1-azatricyclo[6.3.1.0^{4,12}]dodeca-2,4(12),5,7-tetraen-3-yl)-2,2-dimethylpropyl acetate. LC-MS (M+H)⁺=675.5.

### Step 3: methyl (4S)-2-[(2S)-3-(4-{3-[3-(acetyloxy)-2,2-dimethylpropyl]-2{-2-[(1S)-1-methoxyethyl]-5-[4-(propan-2-yl)piperazin-1-yl]pyridin-3-yl}-9-oxa-1-azatricyclo[6.3.1.0^{4,12}]dodeca-2,4(12),5,7-tetraen-6-yl}-1,3-thiazol-2-yl)-2-[{(tert-butoxy)carbonyl]amino}propanoyl]-2,3-diazabicyclo[3.1.1]heptane-4-carboxylate

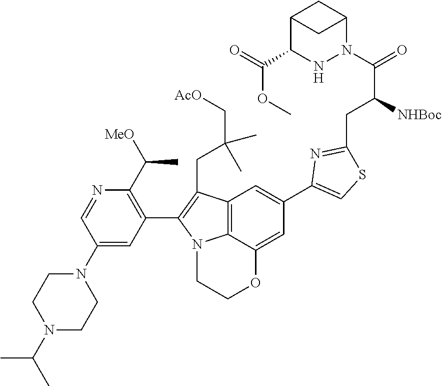 [SMILES: COC(=O)[C@H]1NN(C(=O)[C@H](Cc2nc(-c3cc4c5c(c3)c(CC(C)(C)COC(C)=O)c(-c3cc(N6CCN(C(C)C)CC6)cnc3[C@H](C)OC)n5CCO4)cs2)NC(=O)OC(C)(C)C)C2CC1C2]

The title compound (160 mg, 56%) was prepared in a manner similar to that in example 7 step 4 from 3-(2-{2-[(1S)-1-methoxyethyl]-5-[4-(propan-2-yl)piperazin-1-yl]pyridin-3-yl}-6-(4,4,5,5-tetramethyl-1,3,2-dioxaborolan-2-yl)-9-oxa-1-azatricyclo[6.3.1.0^{4,12}]dodeca-2,4(12),5,7-tetraen-3-yl)-2,2-dimethylpropyl acetate and methyl (4S)-2-[(2S)-3-(4-bromo-1,3-thiazol-2-yl)-2-{[(tert-butoxy)carbonyl]amino}propanoyl]-2,3-diazabicyclo[3.1.1]heptane-4-carboxylate. LC-MS (M+H)⁺=957.3.

### Step 4: (4S)-2-[(2S)-2-{[(tert-butoxy)carbonyl]amino}-3-{4-[3-(3-hydroxy-2,2-dimethylpropyl)-2-{2-[(1S)-1-methoxyethyl]-5-[4-(propan-2-yl)piperazin-1-yl]pyridin-3-yl}-9-oxa-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic Acid

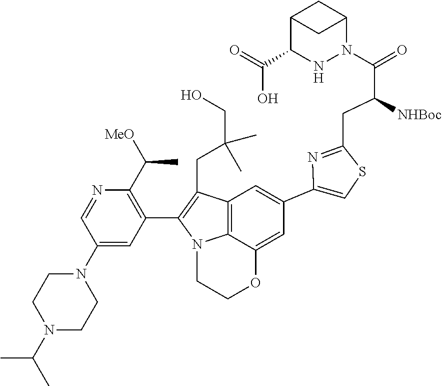 [SMILES: CO[C@@H](C)c1ncc(N2CCN(C(C)C)CC2)cc1-c1c(CC(C)(C)CO)c2cc(-c3csc(C[C@H](NC(=O)OC(C)(C)C)C(=O)N4N[C@H](C(=O)O)C5CC4C5)n3)cc3c2n1CCO3]

The title compound (140 mg, 93%) was prepared in a manner similar to that in example 7 step 5 from methyl (4S)-2-[(2S)-3-(4-{3-[3-(acetyloxy)-2,2-dimethylpropyl]-2-{2-[(1S)-1-methoxyethyl]-5-[4-(propan-2-yl)piperazin-1-yl]pyridin-3-yl}-9-oxa-1-azatricyclo[6.3.1.0^{4,12}]dodeca-2,4(12),5,7-tetraen-6-yl}-1,3-thiazol-2-yl)-2-{[(tert-butoxy)carbonyl]amino}propanoyl]-2,3-diazabicyclo[3.1.1]heptane-4-carboxylate. LC-MS (M+H)⁺=901.6.

### Step 5: tert-butyl N-[(7S,13S)-20-{2-[(1S)-1-methoxyethyl]-5-[4-(propan-2-yl)piperazin-1-yl]pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate

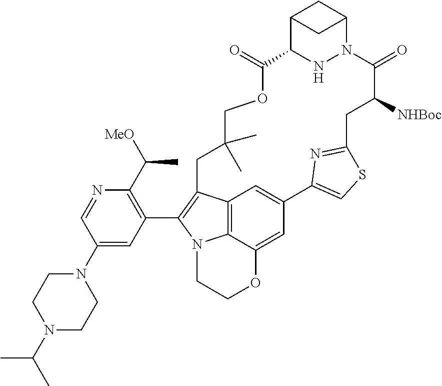 [SMILES: CO[C@@H](C)c1ncc(N2CCN(C(C)C)CC2)cc1-c1c2c3cc(cc4c3n1CCO4)-c1csc(n1)C[C@H](NC(=O)OC(C)(C)C)C(=O)N1N[C@H](C(=O)OCC(C)(C)C2)C2CC1C2]

The title compound (50 mg, 36%) was prepared in a manner similar to that in example 7 step 6 from (4S)-2-[(2S)-2-{[(tert-butoxy)carbonyl]amino}-3-{4-[3-(3-hydroxy-2,2-dimethylpropyl)-2-{2-[(1S)-1-methoxyethyl]-5-[4-(propan-2-yl)piperazin-1-yl]pyridin-3-yl}-9-oxa-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic acid. LC-MS (M+H)⁺=883.5.

### Step 6: (7S,13S)-7-amino-20-{2-[(1S)-1-methoxyethyl]-5-[4-(propan-2-yl)piperazin-1-yl]pyridin-3-yl}-17,17-dimethyl-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione

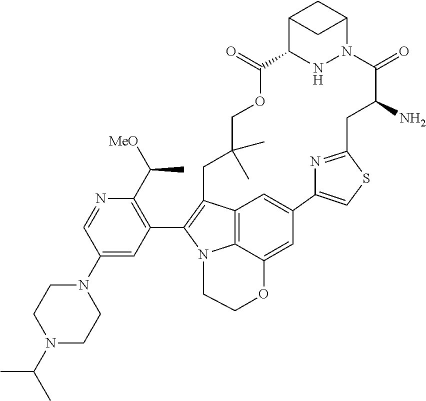 [SMILES: CO[C@@H](C)c1ncc(N2CCN(C(C)C)CC2)cc1-c1c2c3cc(cc4c3n1CCO4)-c1csc(n1)C[C@H](N)C(=O)N1N[C@H](C(=O)OCC(C)(C)C2)C2CC1C2]

The title compound (40 mg, 91%) was prepared in a manner similar to that in example 9 step 7 from tert-butyl N-[(7S,13S)-20-{2-[(1S)-1-methoxyethyl]-5-[4-(propan-2-yl)piperazin-1-yl]pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate. LC-MS (M+H)⁺=783.5.

### Step 7: (1R,2R,3S)—N-[(7S,13S,19P)-20-{2-[(1S)-1-methoxyethyl]-5-[4-(propan-2-yl)piperazin-1-yl]pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide and (1R,2R,3S)—N-[(7S,13S,19M)-20-{2-[(1S)-1-methoxyethyl]-5-[4-(propan-2-yl)piperazin-1-yl]pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

Atrop-25 (P-isomer, 6.0 mg, 13%) and example 25 (M-isomer, 18.0 mg, 40%) were prepared in a manner similar to that in example 9 step 8 from (7S,13S)-7-amino-20-{2-[(1S)-1-methoxyethyl]-5-[4-(propan-2-yl)piperazin-1-yl]pyridin-3-yl}-17,17-dimethyl-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione and (1r,2R,3S)-2,3-dimethylcyclopropane-1-carboxylic acid.

Atrop-25: ¹H NMR (400 MHz, DMSO-d6) δ 8.44-8.38 (m, 2H), 8.04 (s, 1H), 7.75 (s, 1H), 7.35 (s, 1H), 7.13 (s, 1H), 5.85 (d, J=12.0 Hz, 1H), 5.31-5.24 (m, 1H), 4.71 (d, J=12.0 Hz, 1H), 4.52-4.45 (m, 2H), 4.42-4.36 (m, 1H), 4.06-3.96 (m, 2H), 3.67-3.57 (m, 2H), 3.52-3.47 (m, 1H), 3.22-3.16 (m, 4H), 3.14-3.11 (m, 1H), 3.08 (s, 3H), 2.99-2.94 (m, 1H), 2.68-2.61 (m, 2H), 2.56-2.52 (m, 4H), 2.40-2.39 (m, 1H), 2.34-2.28 (m, 2H), 2.13-2.07 (m, 1H), 1.62-1.55 (m, 1H), 1.22-1.19 (m, 2H), 1.14-1.10 (m, 5H), 1.08-1.02 (m, 6H), 0.96 (d, J=6.0 Hz, 6H), 0.91 (s, 3H), 0.47 (s, 3H). LC-MS (M+H)+=879.1.

Example 25: 1H NMR (400 MHz, DMSO-d6) δ 8.44-8.39 (m, 2H), 8.01 (s, 1H), 7.76 (s, 1H), 7.16-7.12 (m, 2H), 5.89 (d, J=12.0 Hz, 1H), 5.34-5.26 (m, 1H), 4.68 (d, J=12.0 Hz, 1H), 4.61-4.51 (m, 2H), 4.48-4.43 (m, 1H), 4.32-4.25 (m, 2H), 3.72-3.65 (m, 1H), 3.57-3.47 (m, 2H), 3.23-3.18 (m, 8H), 3.12-3.04 (m, 1H), 2.91-2.85 (m, 1H), 2.68-2.60 (m, 3H), 2.56-2.53 (m, 4H), 2.31-2.28 (m, 1H), 2.13-2.06 (m, 1H), 1.60-1.54 (m, 1H), 1.30 (d, J=6.0 Hz, 3H), 1.22-1.18 (m, 2H), 1.14-1.11 (m, 2H), 1.07-1.02 (m, 6H), 0.96 (d, J=6.0 Hz, 6H), 0.89 (s, 3H), 0.32 (s, 3H). LC-MS (M+H)+=879.1.

### Example 26: (1R,2R,3S)—N-[(7S,13S,19M)-20-{2-[(1S)-1-methoxyethyl]-5-{5-methyl-5,8-diazaspiro[3.5]nonan-8-yl}pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

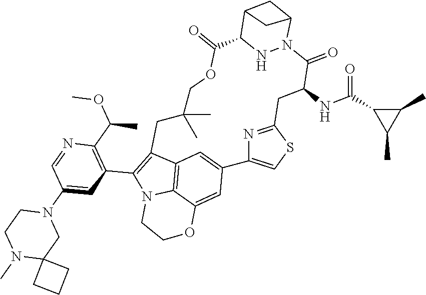

### Step 1: tert-butyl 5-methyl-5,8-diazaspiro[3.5]nonane-8-carboxylate

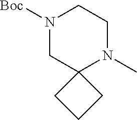 [SMILES: CN1CCN(C(=O)OC(C)(C)C)CC12CCC2]

To a mixture of tert-butyl 5,8-diazaspiro[3.5]nonane-8-carboxylate (1.25 g, 5.5 mmol) in THF (30 mL) was added NaH (265 mg, 6.6 mmol) at 0° C. The mixture was stirred at 0° C. for 30 min, followed by the addition of methyl iodide (942 mg, 6.6 mmol). The mixture was warmed to room temperature and stirred at room temperature for 4 h. The reaction mixture was quenched with water (30 mL) at 0° C. and extracted with EA (30 mL), dried over Na₂SO₄, filtered and concentrated under vacuum to give the title compound (1.33 g, 100%). LC-MS (M+H)⁺=241.1.

### Step 2: 5-methyl-5,8-diazaspiro[3.5]nonane

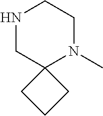

To an round-bottom flask charged with tert-butyl 5-methyl-5,8-diazaspiro[3.5]nonane-8-carboxylate (1.33 g, 5.5 mmol) was added HCl/dioxane (4M, 20 mL). The reaction mixture was stirred at room temperature for 5 h. Volatiles were removed in vacuo to give a residue. The residue was dissolved in saturated aqueous NaHCO₃ (10 mL) and extracted with EA (5×20 mL). The organic phase was separated, dried over Na₂SO₄, filtered and concentrated under vacuum to give the title compound (350 mg, 41%). LC-MS (M+H)⁺=141.1.

### Step 3: 3-(6-bromo-2-{2-[(1S)-1-methoxyethyl]-5-{5-methyl-5,8-diazaspiro[3.5]nonan-8-yl}pyridin-3-yl}-9-oxa-1-azatricyclo[6.3.1.0^{4,12}]dodeca-2,4(12),5,7-tetraen-3-yl)-2,2-dimethylpropyl Acetate

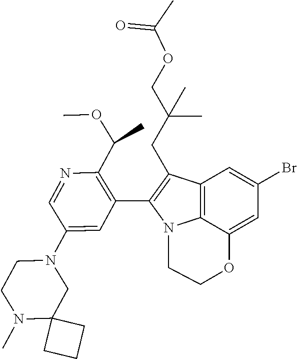 [SMILES: CO[C@@H](C)c1ncc(N2CCN(C)C3(CCC3)C2)cc1-c1c(CC(C)(C)COC(C)=O)c2cc(Br)cc3c2n1CCO3]

The title compound (400 mg, 46%) was prepared in a manner similar to that in example 9 step 2 from 3-(6-bromo-2-{2-[(1S)-1-methoxyethyl]-5-(4,4,5,5-tetramethyl-1,3,2-dioxaborolan-2-yl)pyridin-3-yl}-9-oxa-1-azatricyclo[6.3.1.0^{4,12}]dodeca-2,4(12),5,7-tetraen-3-yl)-2,2-dimethylpropyl acetate and 5-methyl-5,8-diazaspiro[3.5]nonane. LC-MS (M+H)⁺=639.2/641.2.

### Step 4: 3-(2-{2-[(1S)-1-methoxyethyl]-5-{5-methyl-5,8-diazaspiro[3.5]nonan-8-yl}pyridin-3-yl}-6-(4,4,5,5-tetramethyl-1,3,2-dioxaborolan-2-yl)-9-oxa-1-azatricyclo[6.3.1.0^{4,12}]dodeca-2,4(12),5,7-tetraen-3-yl)-2,2-dimethylpropyl Acetate

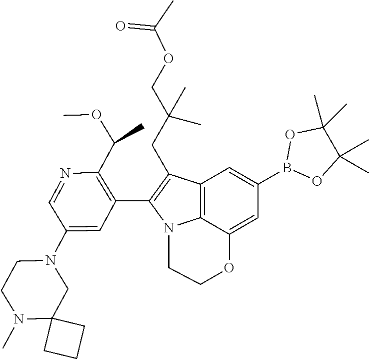 [SMILES: CO[C@@H](C)c1ncc(N2CCN(C)C3(CCC3)C2)cc1-c1c(CC(C)(C)COC(C)=O)c2cc(B3OC(C)(C)C(C)(C)O3)cc3c2n1CCO3]

The title compound (210 mg, 48%) was prepared in a manner similar to that in example 9 step 3 from 3-(6-bromo-2-{2-[(1S)-1-methoxyethyl]-5-{5-methyl-5,8-diazaspiro[3.5]nonan-8-yl}pyridin-3-yl}-9-oxa-1-azatricyclo[6.3.1.0^{4,12}]dodeca-2,4(12),5,7-tetraen-3-yl)-2,2-dimethylpropyl acetate. LC-MS (M+H)⁺=687.4.

### Step 5: methyl (4S)-2-[(2S)-3-(4-{3-[3-(acetyloxy)-2,2-dimethylpropyl]-2-{2-[(1S)-1-methoxyethyl]-5-{5-methyl-5,8-diazaspiro[3.5]nonan-8-yl}pyridin-3-yl}-9-oxa-1-azatricyclo[6.3.1.0^{4,12}]dodeca-2,4(12),5,7-tetraen-6-yl}-1,3-thiazol-2-yl)-2-{[(tert-butoxy)carbonyl]amino}propanoyl]-2,3-diazabicyclo[3.1.1]heptane-4-carboxylate

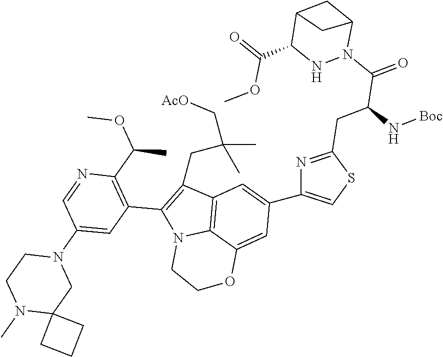 [SMILES: COC(=O)[C@H]1NN(C(=O)[C@H](Cc2nc(-c3cc4c5c(c3)c(CC(C)(C)COC(C)=O)c(-c3cc(N6CCN(C)C7(CCC7)C6)cnc3[C@H](C)OC)n5CCO4)cs2)NC(=O)OC(C)(C)C)C2CC1C2]

The title compound (220 mg, 74%) was prepared in a manner similar to that in example 9 step 4 from 3-(2-{2-[(1S)-1-methoxyethyl]-5-{5-methyl-5,8-diazaspiro[3.5]nonan-8-yl}pyridin-3-yl}-6-(4,4,5,5-tetramethyl-1,3,2-dioxaborolan-2-yl)-9-oxa-1-azatricyclo[6.3.1.0^{4,12}]dodeca-2,4(12),5,7-tetraen-3-yl)-2,2-dimethylpropyl acetate and methyl (4S)-2-[(2S)-3-(4-bromo-1,3-thiazol-2-yl)-2-{[(tert-butoxy)carbonyl]amino}propanoyl]-2,3-diazabicyclo[3.1.1]heptane-4-carboxylate. LC-MS (M+H)⁺=969.4.

### Step 6: (4S)-2-[(2S)-2-{[(tert-butoxy)carbonyl]amino}-3-{4-[3-(3-hydroxy-2,2-dimethylpropyl)-2-{2-[(1S)-1-methoxyethyl]-5-{5-methyl-5,8-diazaspiro[3.5]nonan-8-yl}pyridin-3-yl}-9-oxa-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic Acid

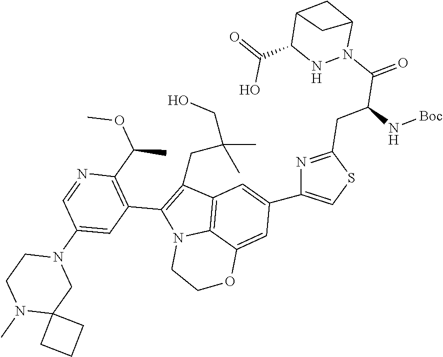 [SMILES: CO[C@@H](C)c1ncc(N2CCN(C)C3(CCC3)C2)cc1-c1c(CC(C)(C)CO)c2cc(-c3csc(C[C@H](NC(=O)OC(C)(C)C)C(=O)N4N[C@H](C(=O)O)C5CC4C5)n3)cc3c2n1CCO3]

The tile compound (157 mg, 76%) was prepared in a manner similar to that in example 9 step 5 from methyl (4S)-2-[(2S)-3-(4-{3-[3-(acetyloxy)-2,2-dimethylpropyl]-2-{2-[(1S)-1-methoxyethyl]-5-(5-methyl-5,8-diazaspiro[3.5]nonan-8-yl}pyridin-3-yl}-9-oxa-1-azatricyclo[6.3.1.0^{4,12}]dodeca-2,4(12),5,7-tetraen-6-yl)-1,3-thiazol-2-yl)-2-{[(tert-butoxy)carbonyl]amino}propanoyl]-2,3-diazabicyclo[3.1.1]heptane-4-carboxylate. LC-MS (M+H)⁺=913.4.

### Step 7: tert-butyl N-[(7S,13S)-20-{2-[(1S)-1-methoxyethyl]-5-{5-methyl-5,8-diazaspiro[3.5]nonan-8-yl}pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate

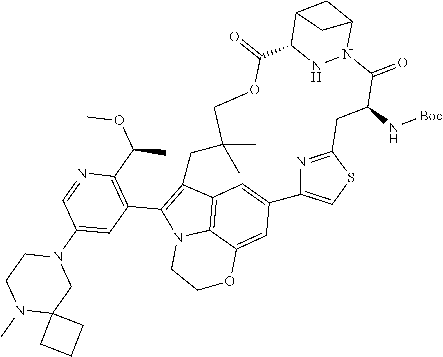 [SMILES: CO[C@@H](C)c1ncc(N2CCN(C)C3(CCC3)C2)cc1-c1c2c3cc(cc4c3n1CCO4)-c1csc(n1)C[C@H](NC(=O)OC(C)(C)C)C(=O)N1N[C@H](C(=O)OCC(C)(C)C2)C2CC1C2]

The title compound (27 mg, 18%) was prepared in a manner similar to that in example 9 step 6 from (4S)-2-[(2S)-2-{[(tert-butoxy)carbonyl]amino}-3-{4-[3-(3-hydroxy-2,2-dimethylpropyl)-2-{2-[(1S)-1-methoxyethyl]-5-{5-methyl-5,8-diazaspiro[3.5]nonan-8-yl}pyridin-3-yl}-9-oxa-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic acid. LC-MS (M+H)⁺=895.4.

### Step 8: (1R,2R,3S)—N-[(7S,13S,19M)-20-{2-[(1S)-1-methoxyethyl]-5-{5-methyl-5,8-diazaspiro[3.5]nonan-8-yl}pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

Example 26 (10 mg, 37%) was prepared in a manner similar to that in example 11 step 6 from tert-butyl N-[(7S,13S)-20-{2-[(1S)-1-methoxyethyl]-5-{5-methyl-5,8-diazaspiro[3.5]nonan-8-yl}pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate and (1r,2R,3S)-2,3-dimethylcyclopropane-1-carboxylic acid. LC-MS (M+H)⁺=891.4. ¹H NMR (400 MHz, DMSO-d6) δ 8.44 (d, J=2.8 Hz, 1H), 8.40 (d, J=9.2 Hz, 1H), 8.02 (s, 1H), 7.75 (s, 1H), 7.21 (d, J=2.6 Hz, 1H), 7.13 (s, 1H), 5.88 (d, J=10.8 Hz, 1H), 5.30 (d, J=8.4 Hz, 1H), 4.68 (d, J=10.6 Hz, 1H), 4.59-4.54 (m, 2H), 4.47 (d, J=5.1 Hz, 1H), 4.33-4.25 (m, 2H), 3.69 (s, 1H), 3.58-3.50 (m, 2H), 3.24-3.15 (m, 9H), 3.11-3.05 (m, 1H), 2.92-2.88 (m, 1H), 2.64 (d, J=1.6 Hz, 3H), 2.30 (s, 2H), 2.24 (s, 3H), 2.20-2.10 (m, 3H), 1.80 (d, J=8.8 Hz, 1H), 1.63-1.53 (m, 4H), 1.31 (d, J=6.1 Hz, 3H), 1.20 (s, 2H), 1.13 (d, J=4.3 Hz, 2H), 1.05 (dd, J=11.1, 5.7 Hz, 6H), 0.89 (s, 3H), 0.34 (s, 3H).

### Example 27: (1R,2R,3S)—N-[(7′S,13′S)-20′-{5-[(9aS)-octahydropyrazino[2,1-c][1,4]oxazin-8-yl]-2-[(1S)-1-methoxyethyl]pyridin-3-yl}-17′,17′-dimethyl-8′,14′-dioxo-15′,24′-dioxa-4′-thia-9′,21′,30′,32′-tetraazaspiro[cyclopropane-1,23′-heptacyclo[23.3.1.1^{2,5}.1^{9,13}.1^{10,12}.0^{19,27}.0^{21,26}]dotriacontane]-1′(28′),2′,5′(32′),19′,25′(29′),26′-hexaen

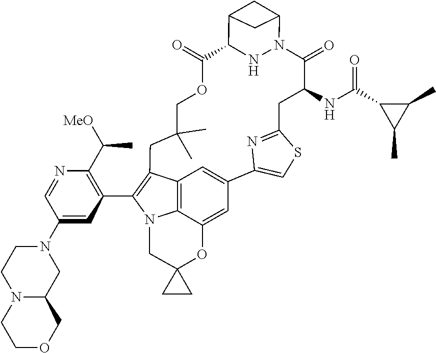

### Step 1: 3-(3′-{5-[(9aS)-octahydropyrazino[2,1-c][1,4]oxazin-8-yl]-2-[(1S)-1-methoxyethyl]pyridin-3-yl}-10′-bromo-7′-oxa-4′-azaspiro[cyclopropane-1,6′-tricyclo[6.3.1.0^{4,12}]dodecane]-1′(12′),2′,8′,10′-tetraen-2′-yl)-2,2-dimethylpropyl Acetate

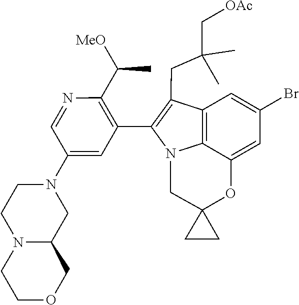 [SMILES: CO[C@@H](C)c1ncc(N2CCN3CCOC[C@@H]3C2)cc1-c1c(CC(C)(C)COC(C)=O)c2cc(Br)cc3c2n1CC1(CC1)O3]

The title compound (340 mg, 60%) was prepared in a manner similar to that in example 1 step 10 from 5-{2′-[3-(acetyloxy)-2,2-dimethylpropyl]-10′-bromo-7′-oxa-4′-azaspiro[cyclopropane-1,6′-tricyclo[6.3.1.0^{4,12}]dodecane]-1′(12′),2′,8′,10′-tetraen-3′-yl}-6-[(1S)-1-methoxyethyl]pyridin-3-yl)boronic acid and (9aS)-octahydropyrazino[2,1-c][1,4]oxazine. LC-MS (M+H)⁺=667.3/669.3.

### Step 2: methyl (4S)-2-[(2S)-2-{[(tert-butoxy)carbonyl]amino}-3-[4-(4,4,5,5-tetramethyl-1,3,2-dioxaborolan-2-yl)-1,3-thiazol-2-yl]propanoyl]-2,3-diazabicyclo[3.1.1]heptane-4-carboxylate

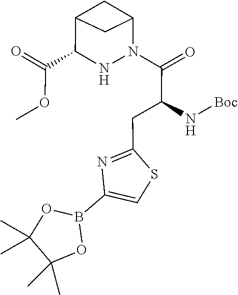 [SMILES: COC(=O)[C@H]1NN(C(=O)[C@H](Cc2nc(B3OC(C)(C)C(C)(C)O3)cs2)NC(=O)OC(C)(C)C)C2CC1C2]

The title compound (450 mg, 37%) was prepared in a manner similar to that in example 9 step 3 from methyl (4S)-2-[(2S)-3-(4-bromo-1,3-thiazol-2-yl)-2-{[(tert-butoxy)carbonyl]amino}propanoyl]-2,3-diazabicyclo[3.1.1]heptane-4-carboxylate and BDP. LC-MS (M-pinacol+2H₂O+H)⁺=455.1.

### Step 3: methyl (4S)-2-[(2S)-3-[4-(3′-{5-[(9aS)-octahydropyrazino[2,1-c][1,4]oxazin-8-yl]-2-[(1S)-1-methoxyethyl]pyridin-3-yl}-2′-[3-(acetyloxy)-2,2-dimethylpropyl]-7′-oxa-4′-azaspiro[cyclopropane-1,6′-tricyclo[6.3.1.0^{4,12}]dodecane]-1′(12′),2′,8′,10′-tetraen-10′-yl)-1,3-thiazol-2-yl]-2-{[(tert-butoxy)carbonyl]amino}propanoyl]-2,3-diazabicyclo[3.1.1]heptane-4-carboxylate

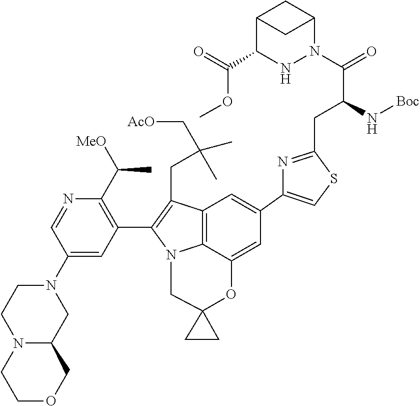 [SMILES: COC(=O)[C@H]1NN(C(=O)[C@H](Cc2nc(-c3cc4c5c(c3)c(CC(C)(C)COC(C)=O)c(-c3cc(N6CCN7CCOC[C@@H]7C6)cnc3[C@H](C)OC)n5CC3(CC3)O4)cs2)NC(=O)OC(C)(C)C)C2CC1C2]

The title compound (270 mg, 54%) was prepared in a manner similar to that in example 9 step 4 from 3-(3′-{5-[(9aS)-octahydropyrazino[2,1-c][1,4]oxazin-8-yl]-2-[(1S)-1-methoxyethyl]pyridin-3-yl}-10′-bromo-7′-oxa-4′-azaspiro[cyclopropane-1,6′-tricyclo[6.3.1.0^{4,12}]dodecane]-1′(12′),2′,8′,10′-tetraen-2′-yl)-2,2-dimethylpropyl acetate and methyl (4S)-2-[(2S)-2-{[(tert-butoxy)carbonyl]amino}-3-[4-(4,4,5,5-tetramethyl-1,3,2-dioxaborolan-2-yl)-1,3-thiazol-2-yl]propanoyl]-2,3-diazabicyclo[3.1.1]heptane-4-carboxylate. LC-MS (M+H)⁺=997.3.

### Step 4: (4S)-2-[(2S)-3-[4-(3′-{5-[(9aS)-octahydropyrazino[2,1-c][1,4]oxazin-8-yl]-2-[(1S)-1-methoxyethyl]pyridin-3-yl}-2′-(3-hydroxy-2,2-dimethylpropyl)-7′-oxa-4′-azaspiro[cyclopropane-1,6′-tricyclo[6.3.1.0^{4,12}]dodecane]-1′(12′),2′,8′,10′-tetraen-10′-yl)-1,3-thiazol-2-yl]-2-{[(tert-butoxy)carbonyl]amino}propanoyl]-2,3-diazabicyclo[3.1.1]heptane-4-carboxylic Acid

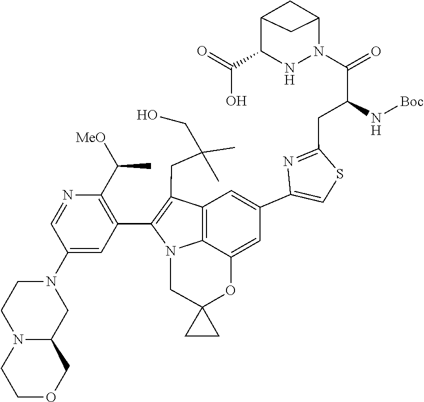 [SMILES: CO[C@@H](C)c1ncc(N2CCN3CCOC[C@@H]3C2)cc1-c1c(CC(C)(C)CO)c2cc(-c3csc(C[C@H](NC(=O)OC(C)(C)C)C(=O)N4N[C@H](C(=O)O)C5CC4C5)n3)cc3c2n1CC1(CC1)O3]

The title compound (200 mg, 79%) was prepared in a manner similar to that in example 9 step 5 from methyl (4S)-2-[(2S)-3-[4-(3′-{5-[(9aS)-octahydropyrazino[2,1-c][1,4]oxazin-8-yl]-2-[(1S)-1-methoxyethyl]pyridin-3-yl}-2′-[3-(acetyloxy)-2,2-dimethylpropyl]-7′-oxa-4′-azaspiro[cyclopropane-1,6′-tricyclo[6.3.1.0^{4,12}]dodecane]-1′(12′),2′,8′,10′-tetraen-10′-yl)-1,3-thiazol-2-yl]-2-{[(tert-butoxy)carbonyl]amino}propanoyl]-2,3-diazabicyclo[3.1.1]heptane-4-carboxylate. LC-MS (M+H)⁺=941.3.

### Step 5: tert-butyl N-[(7′S,13′S)-20′-{5-[(9aS)-octahydropyrazino[2,1-c][1,4]oxazin-8-yl]-2-[(1S)-1-methoxyethyl]pyridin-3-yl}-17′,17′-dimethyl-8′,14′-dioxo-15′,24′-dioxa-4′-thia-9′,21′,30′,32′-tetraazaspiro[cyclopropane-1,23′-heptcyclo[23.3.1.1^{2,5}.1^{9,13}.1^{10,12}.0^{19,27}.0^{21,26}]dotriacontane]-1′(28′),2′,5′(32′),19′,25′(29′),26′-hexaen-7′-yl]carbamate

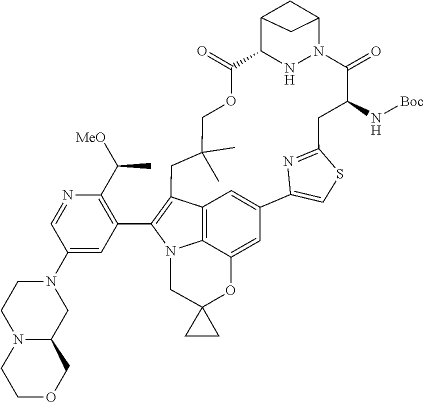 [SMILES: CO[C@@H](C)c1ncc(N2CCN3CCOC[C@@H]3C2)cc1-c1c2c3cc(cc4c3n1CC1(CC1)O4)-c1csc(n1)C[C@H](NC(=O)OC(C)(C)C)C(=O)N1N[C@H](C(=O)OCC(C)(C)C2)C2CC1C2]

The title compound (70 mg, 36%) was prepared in a manner similar to that in example 9 step 6 from (4S)-2-[(2S)-3-[4-(3′-{5-[(9aS)-octahydropyrazino[2,1-c][1,4]oxazin-8-yl]-2-[(1S)-1-methoxyethyl]pyridin-3-yl}-2′-(3-hydroxy-2,2-dimethylpropyl)-7′-oxa-4′-azaspiro[cyclopropane-1,6′-tricyclo[6.3.1.0^{4,12}]dodecane]-1′(12′),2′,8′,10′-tetraen-10′-yl)-1,3-thiazol-2-yl]-2-{[(tert-butoxy)carbonyl]amino}propanoyl]-2,3-diazabicyclo[3.1.1]heptane-4-carboxylic acid. LC-MS (M+H)⁺=923.3.

### Step 6: (7′S,13′S)-20′-{5-[(9aS)-octahydropyrazino[2,1-c][1,4]oxazin-8-yl]-2-[(1S)-1-methoxyethyl]pyridin-3-yl}-7′-amino-17′,17′-dimethyl-15′,24′-dioxa-4′-thia-9′,21′,30′,32′-tetraazaspiro[cyclopropane-1,23′-heptcyclo[23.3.1.1^{2,5}.1^{9,13}.1^{10,12}.0^{19,27}.0^{21,26}]dotriacontane]-1′(28′),2′,5′(32′),19′,25′(29′),26′-hexaene-8′,14′-dione

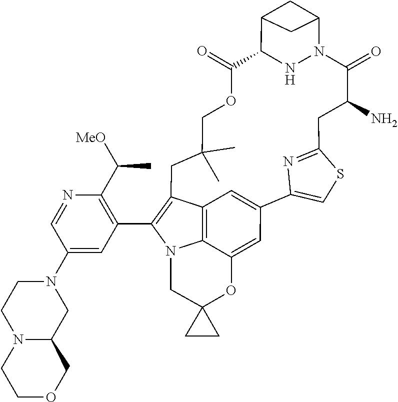 [SMILES: CO[C@@H](C)c1ncc(N2CCN3CCOC[C@@H]3C2)cc1-c1c2c3cc(cc4c3n1CC1(CC1)O4)-c1csc(n1)C[C@H](N)C(=O)N1N[C@H](C(=O)OCC(C)(C)C2)C2CC1C2]

The title compound (62 mg, 100%) was prepared in a manner similar to that in example 9 step 7 from tert-butyl N-[(7′S,13′S)-20′-{5-[(9aS)-octahydropyrazino[2,1-c][1,4]oxazin-8-yl]-2-[(1S)-1-methoxyethyl]pyridin-3-yl}-17′,17′-dimethyl-8′,14′-dioxo-15′,24′-dioxa-4′-thia-9′,21′,30′,32′-tetraazaspiro[cyclopropane-1,23′-heptacyclo[23.3.1.1^{2,5}.1^{9,13}.1^{10,12}.0^{19,27}.0^{21,26}]dotriacontane]-1′(28′),2′,5′(32′),19′,25′(29′),26′-hexaen-7′-yl]carbamate. LC-MS (M+H)⁺=823.3.

### Step 7: (1R,2R,3S)—N-[(7′S,13′S)-20′-{5-[(9aS)-octahydropyrazino[2,1-c][1,4]oxazin-8-yl]-2-[(1S)-1-methoxyethyl]pyridin-3-yl}-17′,17′-dimethyl-8′,14′-dioxo-15′,24′-dioxa-4′-thia-9′,21′,30′,32′-tetraazaspiro[cyclopropane-1,23′-heptacyclo[23.3.1.1^{2,5}.1^{9,13}.1^{10,12}.0^{19,27}.0^{21,26}]dotriacontane]-1′(28′),2′,5′(32′),19′,25′(29′),26′-hexaen

Example 27 (8 mg, 11%) was prepared in a manner similar to that in example 9 step 8 from (7′S,13′S)-20′-{5-[(9aS)-octahydropyrazino[2,1-c][1,4]oxazin-8-yl]-2-[(1S)-1-methoxyethyl]pyridin-3-yl}-7′-amino-17′,17′-dimethyl-15′,24′-dioxa-4′-thia-9′,21′,30′,32′-tetraazaspiro[cyclopropane-1,23′-heptacyclo[23.3.1.1^{2,5}.1^{9,13}.1^{10,12}.0^{19,27}.0^{21,26}]dotriacontane]-1′(28′),2′,5′(32′),19′,25′(29′),26′-hexaene-8′,14′-dione and (1r,2R,3S)-2,3-dimethylcyclopropane-1-carboxylic acid. ¹H NMR (400 MHz, DMSO-d6) δ 8.44-8.42 (m, 2H), 8.08 (s, 1H), 7.76 (s, 1H), 7.21 (s, 1H), 7.14 (s, 1H), 5.95 (d, J=10.7 Hz, 1H), 5.38 (s, 1H), 4.67 (d, J=10.8 Hz, 1H), 4.50-4.48 (m, 2H), 4.04-4.00 (m, 1H), 3.94-3.91 (m, 1H), 3.80-3.75 (m, 2H), 3.69-3.65 (m, 3H), 3.57-3.50 (m, 2H), 3.27-3.20 (m, 1H), 3.19-3.08 (m, 5H), 2.93-2.78 (m, 3H), 2.69-2.63 (m, 2H), 2.47 (brs, 2H), 2.40-2.27 (m, 3H), 2.26-2.11 (m, 3H), 1.55 (t, J=9.0 Hz, 1H), 1.37 (d, J=5.7 Hz, 3H), 1.23-1.21 (m, 2H), 1.17 (s, 2H), 1.08-1.06 (m, 8H), 0.90 (s, 5H), 0.43 (s, 3H). LC-MS (M+H)⁺=919.3

### Example 28: (1R,2R,3S)—N-[(7′S,13′S)-20′-{2-[(1S)-1-methoxyethyl]-5-(2,2,6,6-tetramethyloxan-4-yl)pyridin-3-yl}-17′,17′-dimethyl-8′,14′-dioxo-15′,24′-dioxa-4′-thia-9′,21′,30′,32′-tetraazaspiro[cyclopropane-1,23′-heptacyclo[23.3.1.1^{2,5}.1^{9,13}.1^{10,12}.0^{19,27}.0^{21,26}]dotriacontane]-1′(28′),2′,5′(32′),19′,25′(29′),26′-hexaen-7′-yl]-2,3-dimethylcyclopropane-1-carboxamide

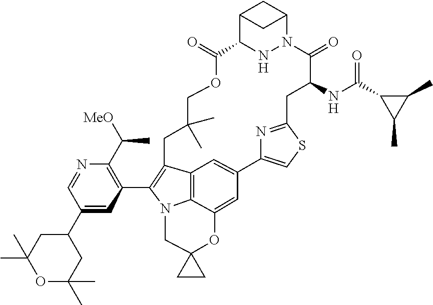

### Step 1: 3-(10′-bromo-3′-{2-[(1S)-1-methoxyethyl]-5-(2,2,6,6-tetramethyloxan-4-yl)pyridin-3-yl}-7′-oxa-4′-azaspiro[cyclopropane-1,6′-tricyclo[6.3.1.0^{4,12}]dodecane]-1′(12′),2′,8′,10′-tetraen-2′-yl)-2,2-dimethylpropyl Acetate

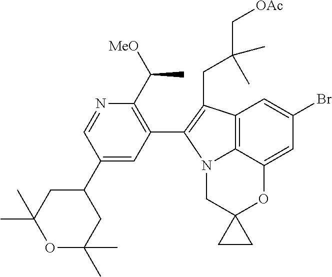 [SMILES: CO[C@@H](C)c1ncc(C2CC(C)(C)OC(C)(C)C2)cc1-c1c(CC(C)(C)COC(C)=O)c2cc(Br)cc3c2n1CC1(CC1)O3]

The title compound (260 mg, 39%) was prepared in a manner similar to that in example 5 step 8 from (5-{2′-[3-(acetyloxy)-2,2-dimethylpropyl]-10′-bromo-7′-oxa-4′-azaspiro[cyclopropane-1,6′-tricyclo[6.3.1.0^{4,12}]dodecane]-1′(12′),2′,8′,10′-tetraen-3′-yl}-6-[(1S)-1-methoxyethyl]pyridin-3-yl)boronic acid and 4-methyl-N′-(2,2,6,6-tetramethyloxan-4-ylidene)benzene-1-sulfonohydrazide. LC-MS (M+H)⁺=669.3/667.3.

### Step 2: methyl (4S)-2-[(2S)-3-(4-{2′-[3-(acetyloxy)-2,2-dimethylpropyl]-3′-{2-[(1S)-1-methoxyethyl]-5-(2,2,6,6-tetramethyloxan-4-yl)pyridin-3-yl}-7′-oxa-4′-azaspiro[cyclopropane-1,6′-tricyclo[6.3.1.0^{4,12}]dodecane]-1′(12′),2′,8′,10′-tetraen-10′-yl}-1,3-thiazol-2-yl)-2-{[(tert-butoxy)carbonyl]amino}propanoyl]-2,3-diazabicyclo[3.1.1]heptane-4-carboxylate

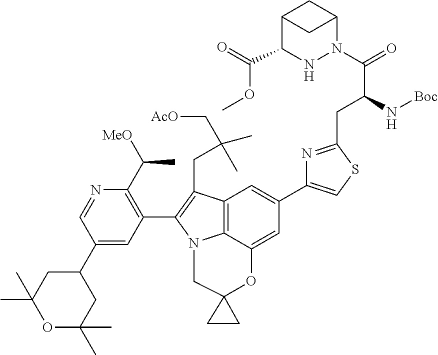 [SMILES: COC(=O)[C@H]1NN(C(=O)[C@H](Cc2nc(-c3cc4c5c(c3)c(CC(C)(C)COC(C)=O)c(-c3cc(C6CC(C)(C)OC(C)(C)C6)cnc3[C@H](C)OC)n5CC3(CC3)O4)cs2)NC(=O)OC(C)(C)C)C2CC1C2]

The title compound (180 mg, 52%) was prepared in a manner similar to that in example 9 step 4 from 3-(10′-bromo-3′-{2-[(1S)-1-methoxyethyl]-5-(2,2,6,6-tetramethyloxan-4-yl)pyridin-3-yl}-7′-oxa-4′-azaspiro[cyclopropane-1,6′-tricyclo[6.3.1.0^{4,12}]dodecane]-1′(12′),2′,8′,10′-tetraen-2′-yl)-2,2-dimethylpropyl acetate and methyl (4S)-2-[(2S)-2-{[(tert-butoxy)carbonyl]amino}-3-[4-(4,4,5,5-tetramethyl-1,3,2-dioxaborolan-2-yl)-1,3-thiazol-2-yl]propanoyl]-2,3-diazabicyclo[3.1.1]heptane-4-carboxylate. LC-MS (M+H)⁺=998.3.

### Step 3: (4S)-2-[(2S)-2-{[(tert-butoxy)carbonyl]amino}-3-{4-[2′-(3-hydroxy-2,2-dimethylpropyl)-3′-{2-[(1S)-1-methoxyethyl]-5-(2,2,6,6-tetramethyloxan-4-yl)pyridin-3-yl}-7′-oxa-4′-azaspiro[cyclopropane-1,6′-tricyclo[6.3.1.0^{4,12}]dodecane]-1′(12′),2′,8′,10′-tetraen-10′-yl]-1,3-thiazol-2-yl}propanoyl]-2,3-diazabicyclo[3.1.1]heptane-4-carboxylic Acid

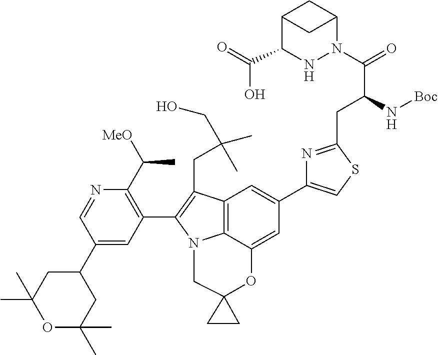 [SMILES: CO[C@@H](C)c1ncc(C2CC(C)(C)OC(C)(C)C2)cc1-c1c(CC(C)(C)CO)c2cc(-c3csc(C[C@H](NC(=O)OC(C)(C)C)C(=O)N4N[C@H](C(=O)O)C5CC4C5)n3)cc3c2n1CC1(CC1)O3]

The title compound (100 mg, 59%) was prepared in a manner similar to that in example 5 step 11 from methyl (4S)-2-[(2S)-3-(4-{2′-[3-(acetyloxy)-2,2-dimethylpropyl]-3′-{2-[(1S)-1-methoxyethyl]-5-(2,2,6,6-tetramethyloxan-4-yl)pyridin-3-yl}-7′-oxa-4′-azaspiro[cyclopropane-1,6′-tricyclo[6.3.1.0^{4,12}]dodecane]-1′(12′),2′,8′,10′-tetraen-10′-yl}-1,3-thiazol-2-yl)-2-{[(tert-butoxy)carbonyl]amino}propanoyl]-2,3-diazabicyclo[3.1.1]heptane-4-carboxylate. LC-MS (M+H)⁺=941.3.

### Step 4: tert-butyl N-[(7′S,13′S)-20′-{2-[(1S)-1-methoxyethyl]-5-(2,2,6,6-tetramethyloxan-4-yl)pyridin-3-yl}-17′,17′-dimethyl-8′,14′-dioxo-15′,24′-dioxa-4′-thia-9′,21′,30′,32′-tetraazaspiro[cyclopropane-1,23′-heptacyclo[23.3.1.1^{2,5}.1^{9,13}.1^{10,12}.0^{19,27}.0^{21,26}]dotriacontane]-1′(28′),2′,5′(32′),19′,25′(29′),26′-hexaen-7′-yl]carbamate

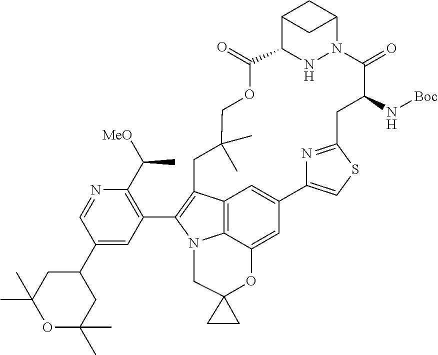 [SMILES: CO[C@@H](C)c1ncc(C2CC(C)(C)OC(C)(C)C2)cc1-c1c2c3cc(cc4c3n1CC1(CC1)O4)-c1csc(n1)C[C@H](NC(=O)OC(C)(C)C)C(=O)N1N[C@H](C(=O)OCC(C)(C)C2)C2CC1C2]

The title compound (70 mg, 69%) was prepared in a manner similar to that in example 5 step 12 from (4S)-2-[(2S)-2-{[(tert-butoxy)carbonyl]amino}-3-{4-[2′-(3-hydroxy-2,2-dimethylpropyl)-3′-{2-[(1S)-1-methoxyethyl]-5-(2,2,6,6-tetramethyloxan-4-yl)pyridin-3-yl}-7′-oxa-4′-azaspiro[cyclopropane-1,6′-tricyclo[6.3.1.0^{4,12}]dodecane]-1′(12′),2′,8′,10′-tetraen-10′-yl]-1,3-thiazol-2-yl}propanoyl]-2,3-diazabicyclo[3.1.1]heptane-4-carboxylic acid. LC-MS (M+H)⁺=923.3.

### Step 5: (7′S,13′S)-7′-amino-20′-{2-[(1S)-1-methoxyethyl]-5-(2,2,6,6-tetramethyloxan-4-yl)pyridin-3-yl}-17′,17′-dimethyl-15′,24′-dioxa-4′-thia-9′,21′,30′,32′-tetraazaspiro[cyclopropane-1,23′-heptacyclo[23.3.1.1^{2,5}.1^{9,13}.1^{10,12}.0^{19,27}.0^{21,26}]dotriacontane]-1′(28′),2′5′(32′),19′,25′(29′),26′-hexaene-8′,14′-dione

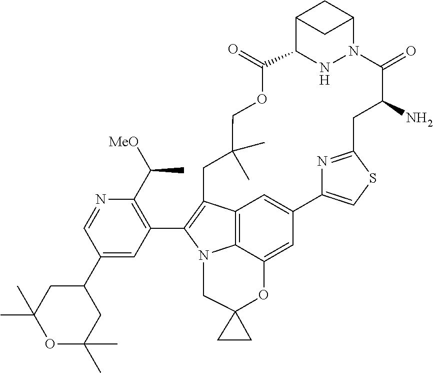 [SMILES: CO[C@@H](C)c1ncc(C2CC(C)(C)OC(C)(C)C2)cc1-c1c2c3cc(cc4c3n1CC1(CC1)O4)-c1csc(n1)C[C@H](N)C(=O)N1N[C@H](C(=O)OCC(C)(C)C2)C2CC1C2]

The title compound (60 mg, 99%) was prepared in a manner similar to that in example 5 step 13 from tert-butyl N-[(7′S,13′S)-20′-(2-[(1S)-1-methoxyethyl]-5-(2,2,6,6-tetramethyloxan-4-yl)pyridin-3-yl)-17′,17′-dimethyl-8′,14′-dioxo-15′,24′-dioxa-4′-thia-9′,21′,30′,32′-tetraazaspiro[cyclopropane-1,23′-heptacyclo[23.3.1.1^{2,5}.1^{9,13}.1^{10,12}.0^{19,27}.0^{21,26}]dotriacontane]-1′(28′),2′,5′(32′),19′,25′(29′),26′-hexaen-7′-yl]carbamate. LC-MS (M+H)⁺=823.3.

### Step 6: (1R,2R,3S)—N-[(7′S,13′S)-20′-{2-[(1S)-1-methoxyethyl]-5-(2,2,6,6-tetramethyloxan-4-yl)pyridin-3-yl}-17′,17′-dimethyl-8′,14′-dioxo-15′,24′-dioxa-4′-thia-9′,21′,30′,32′-tetraazaspiro[cyclopropane-1,23′-heptacyclo[23.3.1.1^{2,5}.1^{9,13}.1^{10,12}.0^{19,27}.0^{21,26}]dotriacontane]-1′(28′),2′,5′(32′),19′,25′(29′),26′-hexaen-7′-yl]-2,3-dimethylcyclopropane-1-carboxamide

Example 28 (8 mg, 11%) was prepared in a manner similar to that in example 5 step 14 from (7′S,13′S)-7′-amino-20′-(2-[(1S)-1-methoxyethyl]-5-(2,2,6,6-tetramethyloxan-4-yl)pyridin-3-yl)-17′,17′-dimethyl-15′,24′-dioxa-4′-thia-9′,21′,30′,32′-tetraazaspiro[cyclopropane-1,23′-heptacyclo[23.3.1.1^{2,5}.1^{9,13}.1^{10,12}.0^{19,27}.0^{21,26}]dotriacontane]-1′(28′),2′,5′(32′),19′,25′(29′),26′-hexaene-8′,14′-dione and (1r,2R,3S)-2,3-dimethylcyclopropane-1-carboxylic acid. ¹H NMR (400 MHz, DMSO-d6) δ 8.67 (s, 1H), 8.42 (d, J=8.7 Hz, 1H), 8.09 (s, 1H), 7.77 (s, 1H), 7.66 (s, 1H), 7.15 (s, 1H), 5.95 (d, J=10.8 Hz, 1H), 5.38 (brs, 1H), 4.69-4.66 (m, 1H), 4.59-4.57 (m, 1H), 4.50 (brs, 1H), 3.99-3.95 (m, 2H), 3.67-3.64 (m, 1H), 3.57-3.54 (m, 1H), 3.20 (s, 3H), 3.15-3.13 (m, 1H), 2.91-2.89 (m, 1H), 2.67-2.63 (m, 4H), 2.33 (s, 3H), 2.15-2.13 (m, 1H), 1.75-1.73 (m, 2H), 1.59-1.42 (m, 4H), 1.40-1.38 (m, 3H), 1.31 (s, 6H), 1.15 (brs, 8H), 1.08-1.05 (m, 7H), 0.92 (s, 2H), 0.88 (s, 3H), 0.39 (s, 3H). LC-MS (M+H)*=919.4.

### Example 29: (1R,2R,3S)—N-[(7S,13S,19M)-20-{2-[(1S)-1-methoxyethyl]-5-[(1S,4S)-5-methyl-2,5-diazabicyclo[2.2.1]heptan-2-yl]pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

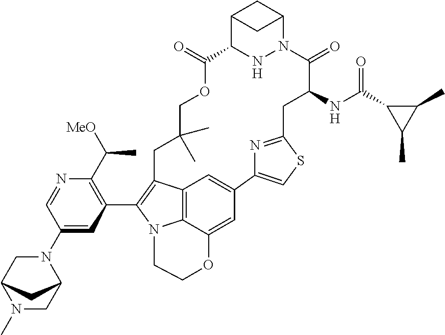

### Step 1: tert-butyl (1S,4S)-5-(5-{3-[3-(acetyloxy)-2,2-dimethylpropyl]-6-bromo-9-oxa-1-azatricyclo[6.3.1.0^{4,12}]dodeca-2,4(12),5,7-tetraen-2-yl}-6-[(1S)-1-methoxyethyl]pyridin-3-yl)-2,5-diazabicyclo[2.2.1]heptane-2-carboxylate

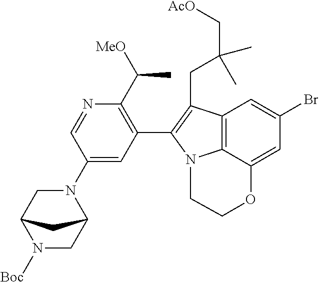 [SMILES: CO[C@@H](C)c1ncc(N2C[C@@H]3C[C@H]2CN3C(=O)OC(C)(C)C)cc1-c1c(CC(C)(C)COC(C)=O)c2cc(Br)cc3c2n1CCO3]

The title compound (460 mg, 28%) was prepared in a manner similar to that in example 7 step 2 from 3-(6-bromo-2-{2-[(1S)-1-methoxyethyl]-5-(4,4,5,5-tetramethyl-1,3,2-dioxaborolan-2-yl)pyridin-3-yl}-9-oxa-1-azatricyclo[6.3.1.0^{4,12}]dodeca-2,4(12),5,7-tetraen-3-yl)-2,2-dimethylpropyl acetate and tert-butyl (1S,4S)-2,5-diazabicyclo[2.2.1]heptane-2-carboxylate. LC-MS (M+H)⁺=697.3/699.3.

### Step 2: 3-(6-bromo-2-{5-[(1S,4S)-2,5-diazabicyclo[2.2.1]heptan-2-yl]-2-[(1S)-1-methoxyethyl]pyridin-3-yl}-9-oxa-1-azatricyclo[6.3.1.0^{4,12}]dodeca-2,4(12),5,7-tetraen-3-yl)-2,2-dimethylpropyl Acetate

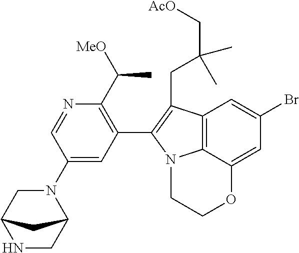 [SMILES: CO[C@@H](C)c1ncc(N2C[C@@H]3C[C@H]2CN3)cc1-c1c(CC(C)(C)COC(C)=O)c2cc(Br)cc3c2n1CCO3]

A mixture of tert-butyl (1S,4S)-5-(5-{3-[3-(acetyloxy)-2,2-dimethylpropyl]-6-bromo-9-oxa-1-azatricyclo[6.3.1.0^{4,12}]dodeca-2,4(12),5,7-tetraen-2-yl}-6-[(1S)-1-methoxyethyl]pyridin-3-yl)-2,5-diazabicyclo[2.2.1]heptane-2-carboxylate (460 mg, 0.66 mmol) and HCl (4 M in dioxane, 2 mL) was stirred for 0.5 h at room temperature. The mixture was concentrated under reduced pressure to give a residue (280 mg, 71%). LC-MS (M+H)⁺=597.3/599.3.

### Step 3: 3-(6-bromo-2-{2-[(1S)-1-methoxyethyl]-5-[(1S,4S)-5-methyl-2,5-diazabicyclo[2.2.1]heptan-2-yl]pyridin-3-yl}-9-oxa-1-azatricyclo[6.3.1.0^{4,12}]dodeca-2,4(12),5,7-tetraen-3-yl)-2,2-dimethylpropyl Acetate

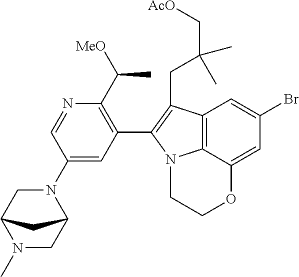 [SMILES: CO[C@@H](C)c1ncc(N2C[C@@H]3C[C@H]2CN3C)cc1-c1c(CC(C)(C)COC(C)=O)c2cc(Br)cc3c2n1CCO3]

To a mixture of 3-(6-bromo-2-{5-[(1S,4S)-2,5-diazabicyclo[2.2.1]heptan-2-yl]-2-[(1S)-1-methoxyethyl]pyridin-3-yl}-9-oxa-1-azatricyclo[6.3.1.0^{4,12}]dodeca-2,4(12),5,7-tetraen-3-yl)-2,2-dimethylpropyl acetate (280 mg, 0.47 mmol) and formaldehyde (1.41 mmol, 141 mg, 30% in water) in MeOH (4 mL) was added STAB (1.41 mmol, 299 mg). The mixture was stirred for 0.5 h at room temperature. Then, saturated aqueous NaHCO₃ solution (30 mL) was added. The mixture was extracted by EtOAc (30 mL×3). The organic layer was concentrated under reduced pressure to give a residue and the residue was purified by silica gel chromatograph (DCM:MeOH=20:1) to give the title compound (240 mg, 84%). LC-MS (M+H)⁺=611.4/613.4.

### Step 4: 3-(2-{2-[(1S)-1-methoxyethyl]-5-(1S,4S)-5-methyl-2,5-diazabicyclo[2.2.1]heptan-2-yl]pyridin-3-yl}-6-(4,4,5,5-tetramethyl-1,3,2-dioxaborolan-2-yl)-9-oxa-1-azatricyclo[6.3.1.0^{4,12}]dodeca-2,4(12),5,7-tetraen-3-yl)-2,2-dimethylpropyl Acetate

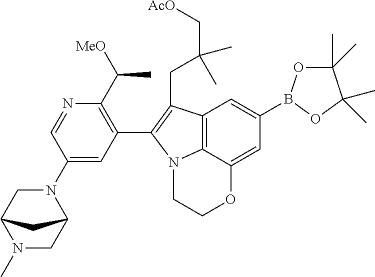 [SMILES: CO[C@@H](C)c1ncc(N2C[C@@H]3C[C@H]2CN3C)cc1-c1c(CC(C)(C)COC(C)=O)c2cc(B3OC(C)(C)C(C)(C)O3)cc3c2n1CCO3]

The title compound (150 mg, 58%) was prepared in a manner similar to that in example 7 step 3 from 3-(6-bromo-2-{2-[(1S)-1-methoxyethyl]-5-[(1S,4S)-5-methyl-2,5-diazabicyclo[2.2.1]heptan-2-yl]pyridin-3-yl}-9-oxa-1-azatricyclo[6.3.1.0^{4,12}]dodeca-2,4(12),5,7-tetraen-3-yl)-2,2-dimethylpropyl acetate. LC-MS (M+H)⁺=659.5.

### Step 5: methyl (4S)-2-[(2S)-3-(4-{3-[3-(acetyloxy)-2,2-dimethylpropyl]-2-{2-[(1S)-1-methoxyethyl]-5-[(1S,4S)-5-methyl-2,5-diazabicyclo[2.2.1]heptan-2-yl]pyridin-3-yl}-9-oxa-1-azatricyclo[6.3.1.0^{4,12}]dodeca-2,4(12),5,7-tetraen-6-yl}-1,3-thiazol-2-yl)-2-{[(tert-butoxy)carbonyl]amino}propanoyl]-2,3-diazabicyclo[3.1.1]heptane-4-carboxylate

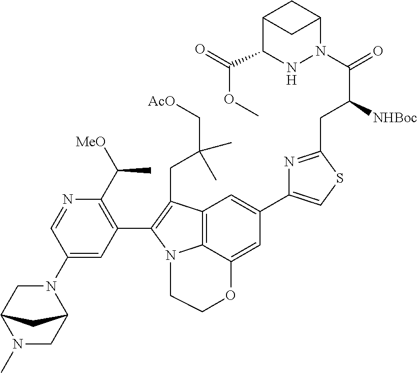 [SMILES: COC(=O)[C@H]1NN(C(=O)[C@H](Cc2nc(-c3cc4c5c(c3)c(CC(C)(C)COC(C)=O)c(-c3cc(N6C[C@@H]7C[C@H]6CN7C)cnc3[C@H](C)OC)n5CCO4)cs2)NC(=O)OC(C)(C)C)C2CC1C2]

The title compound (150 mg, 70%) was prepared in a manner similar to that in example 7 step 4 from 3-(2-(2-[(1S)-1-methoxyethyl]-5-[(1S,4S)-5-methyl-2,5-diazabicyclo[2.2.1]heptan-2-yl]pyridin-3-yl)-6-(4,4,5,5-tetramethyl-1,3,2-dioxaborolan-2-yl)-9-oxa-1-azatricyclo[6.3.1.0^{4,12}]dodeca-2,4(12),5,7-tetraen-3-yl)-2,2-dimethylpropyl acetate and methyl (4S)-2-[(2S)-3-(4-bromo-1,3-thiazol-2-yl)-2-{[(tert-butoxy)carbonyl]amino}propanoyl]-2,3-diazabicyclo[3.1.1]heptane-4-carboxylate. LC-MS (M+H)⁺=941.7.

### Step 6: (4S)-2-[(2S)-2-{[(tert-butoxy)carbonyl]amino}-3-{4-[3-(3-hydroxy-2,2-dimethylpropyl)-2-{2-[(1S)-1-methoxyethyl]-5-[(1S,4S)-5-methyl-2,5-diazabicyclo[2.2.1]heptan-2-yl]pyridin-3-yl}-9-oxa-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic Acid

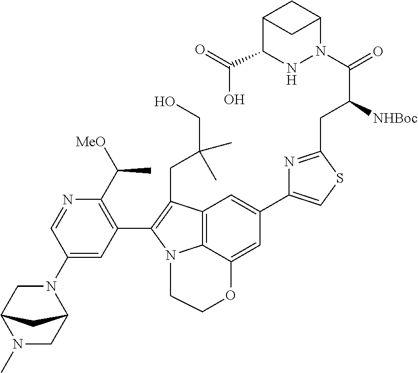 [SMILES: CO[C@@H](C)c1ncc(N2C[C@@H]3C[C@H]2CN3C)cc1-c1c(CC(C)(C)CO)c2cc(-c3csc(C[C@H](NC(=O)OC(C)(C)C)C(=O)N4N[C@H](C(=O)O)C5CC4C5)n3)cc3c2n1CCO3]

The title compound (130 mg, 88%) was prepared in a manner similar to that in example 7 step 5 from methyl (4S)-2-[(2S)-3-(4-{3-[3-(acetyloxy)-2,2-dimethylpropyl]-2-{2-[(1S)-1-methoxyethyl]-5-[(1S,4S)-5-methyl-2,5-diazabicyclo[2.2.1]heptan-2-yl]pyridin-3-yl}-9-oxa-1-azatricyclo[6.3.1.0^{4,12}]dodeca-2,4(12),5,7-tetraen-6-yl}-1,3-thiazol-2-yl)-2-{[(tert-butoxy)carbonyl]amino}propanoyl]-2,3-diazabicyclo[3.1.1]heptane-4-carboxylate. LC-MS (M+H)⁺=885.7.

### Step 7: tert-butyl N-[(7S,13S)-20-{2-[(1S)-1-methoxyethyl]-5-[(1S,4S)-5-methyl-2,5-diazabicyclo[2.2.1]heptan-2-yl]pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate

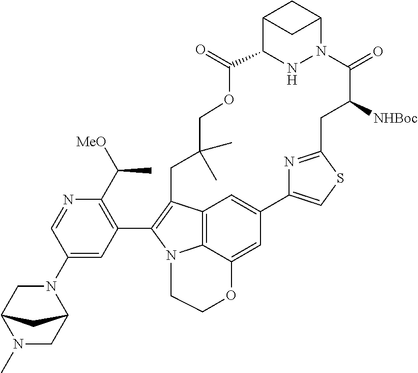 [SMILES: CO[C@@H](C)c1ncc(N2C[C@@H]3C[C@H]2CN3C)cc1-c1c2c3cc(cc4c3n1CCO4)-c1csc(n1)C[C@H](NC(=O)OC(C)(C)C)C(=O)N1N[C@H](C(=O)OCC(C)(C)C2)C2CC1C2]

The title compound (52 mg, 41%) was prepared in a manner similar to that in example 7 step 6 from (4S)-2-[(2S)-2-{[(tert-butoxy)carbonyl]amino}-3-{4-[3-(3-hydroxy-2,2-dimethylpropyl)-2-{2-[(1S)-1-methoxyethyl]-5-[(1S,4S)-5-methyl-2,5-diazabicyclo[2.2.1]heptan-2-yl]pyridin-3-yl}-9-oxa-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic acid. LC-MS (M+H)⁺=867.7.

### Step 8: (1R,2R,3S)—N-[(7S,13S,19M)-20-{2-[(1S)-1-methoxyethyl]-5-[(1S,4S)-5-methyl-2,5-diazabicyclo[2.2.1]heptan-2-yl]pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

Example 29 (18 mg, 35%) was prepared in a manner similar to that in example 7 step 7 from tert-butyl N-[(7S,13S)-20-{2-[(1S)-1-methoxyethyl]-5-[(1S,4S)-5-methyl-2,5-diazabicyclo[2.2.1]heptan-2-yl]pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate and (1r,2R,3S)-2,3-dimethylcyclopropane-1-carboxylic acid. ¹H NMR (400 MHz, DMSO-d6) δ=8.41 (d, J=9.0, 1H), 8.08 (d, J=2.2, 1H), 8.01 (s, 1H), 7.75 (s, 1H), 7.13 (s, 1H), 6.82 (d, J=2.0, 1H), 5.88 (d, J=11.0, 1H), 5.30 (t, J=8.2, 1H), 4.73-4.44 (m, 4H), 4.40-4.22 (m, 3H), 3.70 (t, J=9.0, 1H), 3.60-3.41 (m, 3H), 3.22-3.05 (m, 6H), 2.89 (d, J=14.4, 1H), 2.75-2.60 (m, 2H), 2.43-2.35 (m, 3H), 2.32-2.28 (m, 1H), 2.23 (s, 3H), 2.10 (t, J=9.8, 1H), 1.88-1.73 (m, 2H), 1.57 (t, J=9.2, 1H), 1.30 (d, J=6.0, 3H), 1.24-0.96 (m, 10H), 0.90 (s, 3H), 0.33 (s, 3H). LC-MS (M+H)⁺=863.6.

Atrop-30 & Example 30: (1S,2S)—N-[(7S,13S,19P)-20-{2-[(1S)-1-methoxyethyl]-5-[(3R,5S)-3,4,5-trimethylpiperazin-1-yl]pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2-methylcyclopropane-1-carboxamide & (1S,2S)—N-[(7S,13S,19M)-20-{2-[(1S)-1-methoxyethyl]-5-[(3R,5S)-3,4,5-trimethylpiperazin-1-yl]pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2-methylcyclopropane-1-carboxamide

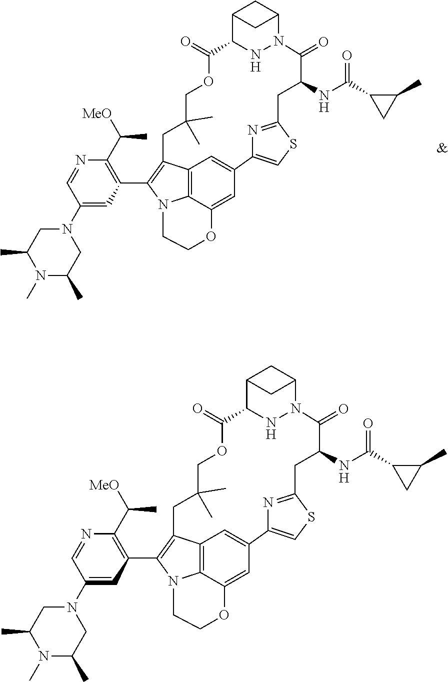

Atrop-30 (P-isomer, 15.2 mg, 13%) and example 30 (M-isomer, 35 mg, 29%) and were prepared in a manner similar to that in example 14 step 6 from tert-butyl N-[(7S,13S)-20-{2-[(1S)-1-methoxyethyl]-5-[(3R,5S)-3,4,5-trimethylpiperazin-1-yl]pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate and (1S,2S)-2-methylcyclopropane-1-carboxylic acid.

Atrop-30: ¹H NMR (400 MHz, DMSO-d₆) δ 8.49 (d, J=8.8, 1H), 8.40 (d, J=2.0, 1H), 8.03 (s, 1H), 7.72 (s, 1H), 7.36 (d, J=2.0, 1H), 7.12 (s, 1H), 5.84 (d, J=11.0, 1H), 5.28 (t, J=8.1, 1H), 4.70 (d, J=11.0, 1H), 4.52-4.33 (m, 3H), 4.08-3.94 (m, 2H), 3.71-3.45 (m, 5H), 3.23-2.92 (m, 6H), 2.63 (d, J=5.5, 1H), 2.41-2.28 (m, 4H), 2.23-2.07 (m, 6H), 1.59 (t, J=9.2, 1H), 1.45 (s, 1H), 1.22-0.95 (m, 14H), 0.93-0.83 (m, 4H), 0.56-0.41 (m, 4H). LC-MS (M+H)⁺=865.6.

Example 30: ¹H NMR (400 MHz, DMSO-d₆) δ 8.49 (d, J=8.8, 1H), 8.39 (d, J=2.6, 1H), 8.00 (s, 1H), 7.72 (s, 1H), 7.15 (d, J=2.6, 1H), 7.11 (s, 1H), 5.87 (d, J=11.0, 1H), 5.31 (t, J=8.0, 1H), 4.72-4.41 (m, 4H), 4.31-4.24 (m, 2H), 3.73-3.43 (m, 5H), 3.23-3.00 (m, 5H), 2.88 (d, J=14.2, 1H), 2.66-2.56 (m, 1H), 2.42-2.03 (m, 10H), 1.60-1.40 (m, 2H), 1.30 (d, J=6.0, 3H), 1.16-0.73 (m, 15H), 0.57-0.45 (m, 1H), 0.32 (s, 3H). LC-MS (M+H)⁺=865.7.

Atrop-31 & Example 31: 1-methylcyclopropyl N-[(7S,13S,19P)-20-{2-[(1S)-1-methoxyethyl]-5-[(3R,5S)-3,4,5-trimethylpiperazin-1-yl]pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate & 1-methylcyclopropyl N-[(7S,13S,19M)-20-{2-[(1S)-1-methoxyethyl]-5-[(3R,5S)-3,4,5-trimethylpiperazin-1-yl]pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate

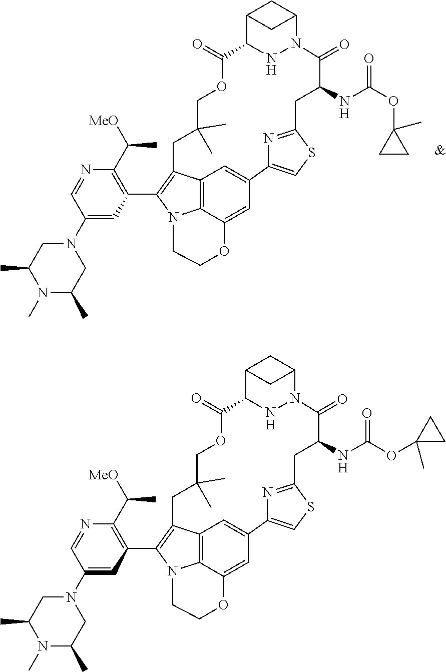

Bis(2,5-dioxopyrrolidin-1-yl) carbonate (82 mg. 0.32 mmol) was added to a solution of 1-methylcyclopropan-1-ol (23 mg. 0.32 mmol) and pyridine (25 mg, 0.32 mmol) in acetonitrile (1.5 mL) under nitrogen. The reaction mixture was stirred at 40° C. for 18 h before cooling to room temperature. (7S,13S)-7-amino-20-{2-[(1S)-1-methoxyethyl]-5-[(3R,5S)-3,4,5-trimethylpiperazin-1-yl]pyridin-3-yl}-17,17-dimethyl-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione (78 mg, 0.1 mmol), which was prepared in a manner similar to that in example 9 step 7 from tert-butyl N-[(7S,13S)-20-{2-[(1S)-1-methoxyethyl]-5-[(3R,5S)-3,4,5-trimethylpiperazin-1-yl]pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate (88 mg, 0.1 mmol), was dissolved in DMF (2 mL) and the solution was added to the above reaction mixture, followed by the addition of TEA (32 mg, 0.32 mmol). The reaction mixture was stirred at room temperature for 4 h and then quenched with brine (30 mL) and extracted by EtOAc (30 mL×3). The combined organic layer was dried over Na₂SO₄, filtered and concentrated under vacuum. The residue was purified by prep-HPLC to give atrop-31 (P-isomer, 12 mg, 14%) and example 31 (M-isomer, 35 mg, 40%).

Atrop-31: ¹H NMR (400 MHz, DMSO-d₆) δ 8.41 (d, J=2.0, 1H), 8.06 (s, 1H), 7.77 (s, 1H), 7.53 (d, J=9.2, 1H), 7.37 (d, J=2.0, 1H), 7.15 (s, 1H), 5.89 (d, J=11.0, 1H), 4.99 (t, J=7.6, 1H), 4.72 (d, J=10.8, 1H), 4.52-4.33 (m, 3H), 4.08-3.95 (m, 2H), 3.72-3.47 (m, 5H), 3.18-2.93 (m, 6H), 2.66-2.61 (m 1H), 2.42-2.28 (m, 3H), 2.24-2.11 (m, 6H), 1.59 (t, J=9.2, 1H), 1.44 (s, 3H), 1.40-1.30 (m, 1H), 1.23-1.17 (m, 1H), 1.12 (d, J=6.0, 3H), 1.02 (d, J=6.0, 6H), 0.92 (s, 3H), 0.80-0.70 (m, 2H), 0.66-0.54 (m, 2H), 0.47 (s, 3H). LC-MS (M+H)⁺=881.8.

Example 31: ¹H NMR (400 MHz, DMSO-d₆) δ 8.41 (d, J=2.8, 1H), 8.03 (s, 1H), 7.77 (s, 1H), 7.53 (d, J=9.1, 1H), 7.19-7.12 (m, 2H), 5.92 (d, J=11.0, 1H), 5.00 (t, J=8.0, 1H), 4.70 (d, J=11.0, 1H), 4.62-4.45 (m, 3H), 4.35-4.23 (m, 2H), 3.74-3.63 (m, 3H), 3.54 (dd, J=26.4, 10.6, 2H), 3.23-3.07 (m, 5H), 2.91 (d, J=14.4, 1H), 2.67-2.60 (m, 1H), 2.45-2.37 (m, 3H), 2.35-2.06 (m, 8H), 1.58 (t, J=9.2, 1H), 1.44 (s, 3H), 1.31 (d, J=6.0, 3H), 1.02 (dd, J=6.0, 2.7, 6H), 0.91 (s, 3H), 0.77-0.71 (m, 2H), 0.63-0.53 (m, 2H), 0.33 (s, 3H). LC-MS (M+H)⁺=881.7.

Atrop-32 & Example 32: (1R,2R,3S)—N-[(7S,13S,19P)-20-{2-[(1S)-1-methoxyethyl]-5-{4-methyl-4,7-diazaspiro[2.5]octan-7-yl}pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide & (1R,2R,3S)—N-[(7S,13S,19M)-20-{2-[(1S)-1-methoxyethyl]-5-{4-methyl-4,7-diazaspiro[2.5]octan-7-yl}pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

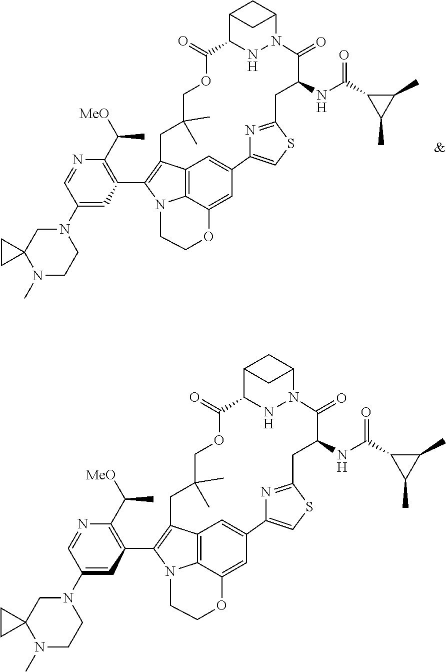

### Step 1: methyl (4S)-2-[(2S)-2-amino-3-(4-bromo-1,3-thiazol-2-yl)propanoyl]-2,3-diazabicyclo[3.1.1]heptane-4-carboxylate Hydrochloride

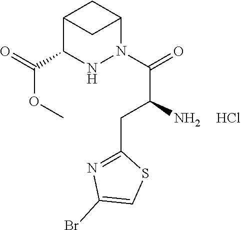 [SMILES: COC(=O)[C@H]1NN(C(=O)[C@@H](N)Cc2nc(Br)cs2)C2CC1C2 ; Cl]

To the solution of methyl (4S)-2-[(2S)-3-(4-bromo-1,3-thiazol-2-yl)-2-{[(tert-butoxy)carbonyl]amino}propanoyl]-2,3-diazabicyclo[3.1.1]heptane-4-carboxylate (1.0 g, 2.0 mmol) in DCM (20 mL) was added a solution of HCl in 1,4-dioxane (5 mL, 4 M) and the solution was stirred at room temperature for 2 h. Then, the reaction mixture was concentrated under vacuum to give the title product which was used in the next step without further purification. LC-MS (M+H)⁺=389.1/391.1.

### Step 2: methyl (4S)-2-[(2S)-3-(4-bromo-1,3-thiazol-2-yl)-2-{[(1R,2R,3S)-2,3-dimethylcyclopropyl]formamido}propanoyl]-2,3-diazabicyclo[3.1.1]heptane-4-carboxylate

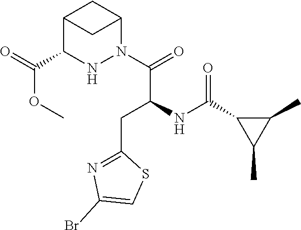 [SMILES: COC(=O)[C@H]1NN(C(=O)[C@H](Cc2nc(Br)cs2)NC(=O)[C@H]2[C@H](C)[C@@H]2C)C2CC1C2]

The reaction mixture of methyl (4S)-2-[(2S)-2-amino-3-(4-bromo-1,3-thiazol-2-yl)propanoyl]-2,3-diazabicyclo[3.1.1]heptane-4-carboxylate hydrochloride (crude), (1r,2R,3S)-2,3-dimethylcyclopropane-1-carboxylic acid (456 mg, 4 mmol), HATU (1.52 g, 4 mmol) and DIPEA (774 mg, 6 mmol) in DMF (15 mL) was stirred at room temperature overnight. The mixture was treated with water (150 mL) and extracted with EA (20 mL×3). The combined organic layers were washed with brine (30 mL), dried over Na₂SO₄, filtered and evaporated. The residue was purified by column chromatography (EA/PE=1/4) to give the title compound (970 mg, 100%). LC-MS (M+H)⁺=485.2/487.2.

### Step 3: (4S)-2-[(2S)-3-(4-bromo-1,3-thiazol-2-yl)-2-{[(1R,2R,3S)-2,3-dimethylcyclopropyl]formamido}propanoyl]-2,3-diazabicyclo[3.1.1]heptane-4-carboxylic Acid

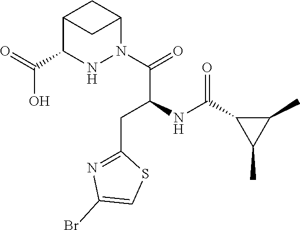 [SMILES: C[C@@H]1[C@H](C)[C@H]1C(=O)N[C@@H](Cc1nc(Br)cs1)C(=O)N1N[C@H](C(=O)O)C2CC1C2]

To a solution of methyl (4S)-2-[(2S)-3-(4-bromo-1,3-thiazol-2-yl)-2-{[(1R,2R,3S)-2,3-dimethylcyclopropyl]formamido}propanoyl]-2,3-diazabicyclo[3.1.1]heptane-4-carboxylate (970 mg, 2 mmol) in THF (10 mL) was added a solution of LiOH (252 mg, 6 mmol) in water (3 mL) and the mixture was stirred at room temperature overnight. The reaction was quenched with aqueous solution of HCl (1 M) and THF was evaporated. The precipitate was filtered and dried to give the title compound (890 mg, 94%). ¹H NMR (400 MHz, DMSO-d₆) δ 7.98 (d, J=7.9 Hz, 1H), 7.66 (s, 1H), 5.89 (s, 1H), 5.50 (dd, J=13.5, 7.5 Hz, 1H), 4.46 (dd, J=10.1, 4.7 Hz, 1H), 3.57 (s, 1H), 3.35 (d, J=5.7 Hz, 1H), 3.20-3.09 (m, 2H), 2.31-2.19 (m, 1H), 2.18-2.13 (m, 1H), 1.80 (t, J=9.5 Hz, 1H), 1.43 (t, J=9.1 Hz, 1H), 1.13 (s, 3H), 1.00-0.98 (m, 6H). LC-MS (M+H)⁺=471.2, 473.2.

### Step 4: tert-butyl 7-(5-{3-[3-(acetyloxy)-2,2-dimethylpropyl]-6-bromo-9-oxa-1-azatricyclo[6.3.1.0^{4,12}]dodeca-2,4(12),5,7-tetraen-2-yl}-6-[(1S)-1-methoxyethyl]pyridin-3-yl)-4,7-diazaspiro[2.5]octane-4-carboxylate

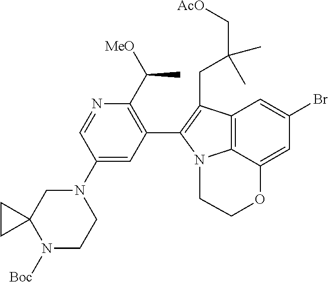 [SMILES: CO[C@@H](C)c1ncc(N2CCN(C(=O)OC(C)(C)C)C3(CC3)C2)cc1-c1c(CC(C)(C)COC(C)=O)c2cc(Br)cc3c2n1CCO3]

To a solution of (5-{3-[3-(acetyloxy)-2,2-dimethylpropyl]-6-bromo-9-oxa-1-azatricyclo[6.3.1.0^{4,12}]dodeca-2,4(12),5,7-tetraen-2-yl}-6-[(1S)-1-methoxyethyl]pyridin-3-yl)boronic acid (1.58 g, 2.9 mmol) in MeCN (20 mL) was added tert-butyl 4,7-diazaspiro[2.5]octane-4-carboxylate (3.1 g, 14.5 mmol), Cu(OAc)₂ (1.06 g, 5.8 mmol) and Et₃N (879 mg, 8.7 mmol). The mixture was stirred at room temperature overnight under oxygen. The mixture was diluted with brine (50 mL) and DCM (50 mL). The solids was filtered off, and the filter cake was rinsed with DCM:MeOH (10:1, 300 mL). The organic layer of the filtrate was separated and dried over Na₂SO₄, filtered and concentrated under vacuum. The residue was purified by silica gel chromatograph (DCM:MeOH=10:1) to give the title compound (1 g, 48%). LC-MS (M+H)⁺=711.6/713.6.

### Step 5: 3-[6-bromo-2-(5-{4,7-diazaspiro[2.5]octan-7-yl}-2-[(1S)-1-methoxyethyl]pyridin-3-yl)-9-oxa-1-azatricyclo[6.3.1.0^{4,12}]dodeca-2,4(12),5,7-tetraen-3-yl]-2,2-dimethylpropan-1-ol

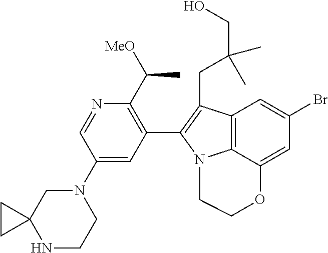

To the solution of tert-butyl 7-(5-{3-[3-(acetyloxy)-2,2-dimethylpropyl]-6-bromo-9-oxa-1-azatricyclo[6.3.1.0^{4,12}]dodeca-2,4(12),5,7-tetraen-2-yl}-6-[(1S)-1-methoxyethyl]pyridin-3-yl)-4,7-diazaspiro[2.5]octane-4-carboxylate (1.0 g, 1.4 mmol) in DCM (10 mL) was added a solution of HCl in 1,4-dioxane (5 mL, 4 M) and the mixture was stirred at room temperature overnight. The solvent was evaporated to give the title compound which was used in the next step without further purification. LC-MS (M+H)⁺=569.4/571.4.

### Step 6: 3-(6-bromo-2-{2-[(1S)-1-methoxyethyl]-5-{4-methyl-4,7-diazaspiro[2.5]octan-7-yl}pyridin-3-yl}-9-oxa-1-azatricyclo[6.3.1.0^{4,12}]dodeca-2,4(12),5,7-tetraen-3-yl)-2,2-dimethylpropan-1-ol

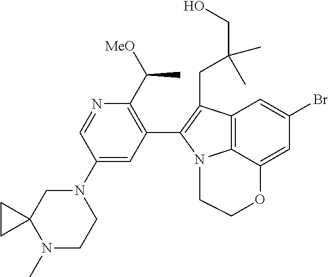

To the solution of 3-[6-bromo-2-(5-{4,7-diazaspiro[2.5]octan-7-yl}-2-[(1S)-1-methoxyethyl]pyridin-3-yl)-9-oxa-1-azatricyclo[6.3.1.0^{4,12}]dodeca-2,4(12),5,7-tetraen-3-yl]-2,2-dimethylpropan-1-ol (crude, 1.4 mmol) in MeOH/DCM (5 mL/15 mL) was added a solution of formaldehyde in water (4 mL, 37%), followed by the addition of NaBH(CN)₃ (441 mg, 7 mmol). The reaction mixture was stirred at room temperature for 5 h. The reaction mixture was quenched with a saturated aqueous solution of NaHCO₃ (10 mL) and the mixture was extracted with DCM (50 mL*3). The combined organic layers were washed with brine (20 mL), dried over Na₂SO₄, filtered and evaporated. The residue was purified by column chromatography (DCM/MeOH=20/1) to give the title compound (800 mg, 97%). LC-MS (M+H)⁺=583.4/585.4.

### Step 7: [3-(3-hydroxy-2,2-dimethylpropyl)-2-{2-[(1S)-1-methoxyethyl]-5-{4-methyl-4,7-diazaspiro[2.5]octan-7-yl}pyridin-3-yl}-9-oxa-1-azatricyclo[6.3.1.0^{4,12}]dodeca-2,4(12),5,7-tetraen-6-yl]boronic Acid

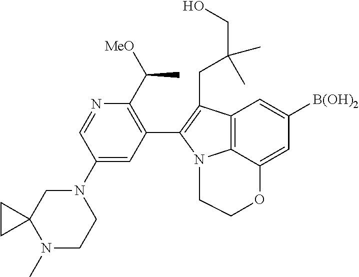

The reaction mixture of 3-(6-bromo-2-{2-[(1S)-1-methoxyethyl]-5-{4-methyl-4,7-diazaspiro[2.5]octan-7-yl}pyridin-3-yl}-9-oxa-1-azatricyclo[6.3.1.0^{4,12}]dodeca-2,4(12),5,7-tetraen-3-yl)-2,2-dimethylpropan-1-ol (800 mg, 1.36 mmol), B₂(OH)₄ (353 mg, 3.92 mmol), Xphos-Pd-G3 (118 mg, 0.14 mmol), Xphos (134 mg, 0.28 mmol) and KOPiv (437 mg, 3.08 mmol) in Me-THF/MeOH (21 mL/7 mL) under nitrogen was stirred at 30° C. for 1 h. The reaction mixture was concentrated under vacuum and the residue was purified by reverse-phase column chromatography to give the product (490 mg, 65%). LC-MS (M+H)⁺=549.6.

### Step 8: (4S)-2-[(2S)-2-{[(1R,2R,3S)-2,3-dimethylcyclopropyl]formamido}-3-{4-[3-(3-hydroxy-2,2-dimethylpropyl)-2-{2-[(1S)-1-methoxyethyl]-5-{4-methyl-4,7-diazaspiro[2.5]octan-7-yl}pyridin-3-yl}-9-oxa-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic Acid

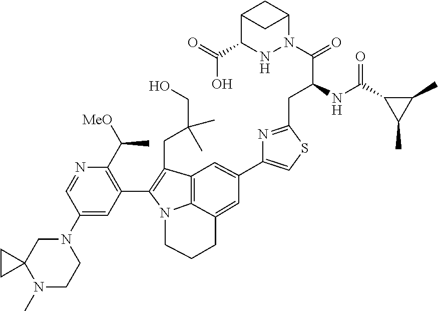 [SMILES: CO[C@@H](C)c1ncc(N2CCN(C)C3(CC3)C2)cc1-c1c(CC(C)(C)CO)c2cc(-c3csc(C[C@H](NC(=O)[C@H]4[C@H](C)[C@@H]4C)C(=O)N4N[C@H](C(=O)O)C5CC4C5)n3)cc3c2n1CCC3]

The reaction mixture of [3-(3-hydroxy-2,2-dimethylpropyl)-2-(2-[(1S)-1-methoxyethyl]-5-(4-methyl-4,7-diazaspiro[2.5]octan-7-yl)pyridin-3-yl)-9-oxa-1-azatricyclo[6.3.1.0^{4,12}]dodeca-2,4(12),5,7-tetraen-6-yl]boronic acid (460 mg, 0.84 mmol), (4S)-2-[(2S)-3-(4-bromo-1,3-thiazol-2-yl)-2-{[(1R,2R,3S)-2,3-dimethylcyclopropyl]formamido}propanoyl]-2,3-diazabicyclo[3.1.1]heptane-4-carboxylic acid (434 mg, 0.92 mmol), Pd(dtbpf)Cl₂ (55 mg, 0.084 mmol) and K₂CO₃ (348 mg, 2.52 mmol) in 1,4-dioxane/H₂O (25 mL/4 mL) under nitrogen was stirred at 90° C. for 2 h. The solvent was evaporated, and the residue was dissolved with DCM (40 mL) and water (40 mL). The organic layer was separated, and the aqueous phase was extracted with DCM (50 mL×3). The combined organic layers were washed with brine (40 mL), dried over Na₂SO₄, filtered and evaporated. The residue was purified by prep-TLC (DCM/MeOH=12/1) to give the title compound (550 mg, 73%). LC-MS (M+H)⁺=895.7.

### Step 9: (1R,2R,3S)—N-[(7S,13S,19P)-20-{2-[(1S)-1-methoxyethyl]-5-{4-methyl-4,7-diazaspiro[2.5]octan-7-yl}pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide & (1R,2R,3S)—N-[(7S,13S,19M)-20-{2-[(1S)-1-methoxyethyl]-5-{4-methyl-4,7-diazaspiro[2.5]octan-7-yl}pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

The reaction mixture of (4S)-2-[(2S)-2-{[(R,2R,3S)-2,3-dimethylcyclopropyl]formamido}-3-{4-[3-(3-hydroxy-2,2-dimethylpropyl)-2-{2-[(1S)-1-methoxyethyl]-5-{4-methyl-4,7-diazaspiro[2.5]octan-7-yl}pyridin-3-yl}-9-oxa-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic acid (550 mg, 0.61 mmol), EDCI (354 mg, 1.84 mmol), HOBT (165 mg, 1.22 mmol), DIPEA (157 mg, 1.22 mmol) and DMAP (37 mg, 0.31 mmol) in dry DCM (60 mL) under nitrogen was stirred at room temperature overnight. The mixture was washed with brine (40 mL), dried over Na₂SO₄, filtered and evaporated. The residue was purified by column chromatography (DCM/MeOH=12/1) and SFC to give atrop-32 (P-isomer, 24 mg, 4.5%) and example 32 (M-isomer, 86 mg, 16%).

Atrop-32: ¹H NMR (400 MHz, DMSO-d6) δ 8.40-8.37 (m, 2H), 8.04 (s, 1H), 7.74 (s, 1H), 7.35 (s, 1H), 7.13 (s, 1H), 5.84 (d, J=10.9 Hz, 1H), 5.28 (t, J=7.9 Hz, 1H), 4.71 (d, J=10.9 Hz, 1H), 4.48-4.38 (m, 3H), 4.10-3.93 (m, 2H), 3.71-3.43 (m, 3H), 3.27-3.20 (m, 2H), 3.20-3.12 (m, 2H), 3.08-3.05 (m, 4H), 3.03-2.93 (m, 2H), 2.89 (s, 2H), 2.65-2.64 (m, 1H), 2.51-2.50 (m, 4H), 2.35-2.29 (m, 1H), 2.25 (s, 3H), 2.11 (t, J=9.8 Hz, 1H), 1.59 (t, J=9.2 Hz, 1H), 1.26-1.18 (m, 1H), 1.17-1.09 (m, 5H), 1.07-1.03 (m, 6H), 0.91 (s, 3H), 0.58 (s, 2H), 0.48 (s, 3H). LC-MS (M+H)⁺=877.7.

Example 32: ¹H NMR (400 MHz, DMSO-d6) δ 8.40-8.37 (m, 2H), 8.01 (s, 1H), 7.74 (s, 1H), 7.13 (s, 2H), 5.87 (d, J=11.0 Hz, 1H), 5.31 (t, J=7.9 Hz, 1H), 4.67 (d, J=11.1 Hz, 1H), 4.64-4.41 (m, 3H), 4.37-4.19 (m, 2H), 3.77-3.45 (m, 3H), 3.22-3.19 (m, 6H), 3.11-3.07 (m, 3H), 2.90 (s, 4H), 2.62 (d, J=5.2 Hz, 2H), 2.51 (s, 4H), 2.37-2.19 (m, 6H), 2.15-2.06 (m, 1H), 1.56 (s, 1H), 1.31 (d, J=5.9 Hz, 3H), 1.22-1.11 (m, 3H), 1.08-1.00 (m, 5H), 0.90 (s, 3H), 0.58 (s, 2H), 0.46 (s, 2H), 0.35 (s, 3H). LC-MS (M+H)⁺=877.7.

Atrop-33 & Example 33: (1S,2S)—N-[(7S,13S,19P)-20-{2-[(1S)-1-methoxyethyl]-5-(4-methylpiperazin-1-yl)pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2-methylcyclopropane-1-carboxamide and (1S,2S)—N-[(7S,13S,19M)-20-{2-[(1S)-1-methoxyethyl]-5-(4-methylpiperazin-1-yl)pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2-methylcyclopropane-1-carboxamide

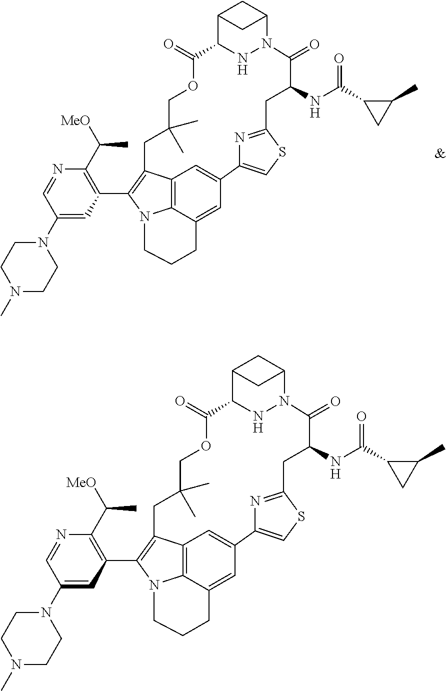

Atrop-33 (P-isomer, 26 mg, 14.8%) and example 33 (M-isomer, 16 mg, 9%) were prepared in a manner similar to that in example 21 from (7S,13S)-7-amino-20-{2-[(1S)-1-methoxyethyl]-5-(4-methylpiperazin-1-yl)pyridin-3-yl}-17,17-dimethyl-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione and (1S,2S)-2-methylcyclopropane-1-carboxylic acid, and the atropisomers were separated by prep-HPLC.

Atrop-33: ¹H NMR (400 MHz, DMSO) δ 8.56 (d, J=8.9 Hz, 1H), 8.44 (d, J=2.7 Hz, 1H), 8.27 (s, 1H), 7.73 (s, 1H), 7.44 (s, 1H), 7.37 (d, J=2.7 Hz, 1H), 5.91 (d, J=11.1 Hz, 1H), 5.33 (t, J=8.2 Hz, 1H), 4.75 (d, J=11.0 Hz, 1H), 4.51 (dd, J=9.5, 4.6 Hz, 1H), 3.91 (m, 2H), 3.63 (d, J=10.7 Hz, 1H), 3.54 (d, J=10.8 Hz, 1H), 3.51-3.42 (m, 1H), 3.31-3.12 (m, 6H), 3.08 (s, 3H), 3.04-2.93 (m, 3H), 2.68 (q, J=5.8 Hz, 1H), 2.45 (t, J=4.8 Hz, 4H), 2.39-2.34 (m, 3H), 2.26-2.13 (m, 5H), 2.08-1.97 (m, 1H), 1.63 (t, J=9.3 Hz, 1H), 1.54-1.48 (m, 1H), 1.13 (d, J=6.3 Hz, 3H), 1.08 (s, 4H), 0.94-0.88 (m, 4H), 0.61-0.48 (m, 4H).

LC-MS (M+H)⁺=835.3.

Example 33: ¹H NMR (400 MHz, DMSO-d6) δ 8.53 (d, J=9.0 Hz, 1H), 8.44 (d, J=2.6 Hz, 1H), 8.24 (s, 1H), 7.72 (s, 1H), 7.44 (s, 1H), 7.15 (d, J=2.6 Hz, 1H), 5.94 (d, J=11.0 Hz, 1H), 5.38 (d, J=7.9 Hz, 1H), 4.68 (d, J=11.0 Hz, 1H), 4.51-4.48 (m, 1H), 4.32-4.14 (m, 2H), 3.57 (dd, J=28.2, 10.8 Hz, 3H), 3.28-3.22 (m, 7H), 3.12 (dd, J=14.8, 7.4 Hz, 1H), 3.13-3.01 (m, 1H), 2.90 (d, J=14.5 Hz, 2H), 2.69-2.62 (m, 3H), 2.46-2.40 (m, 5H), 2.36-2.31 (m, 2H), 2.21 (s, 3H), 2.18-2.10 (m, 2H), 1.57 (t, J=9.4 Hz, 1H), 1.51-1.48 (m, 1H), 1.34 (d, J=6.0 Hz, 3H), 1.07 (s, 4H), 0.90 (s, 4H), 0.57-0.54 (m, 1H), 0.39 (s, 3H). LC-MS (M+H)⁺=835.3.

Atrop-34 & Example 34: (1R,2R,3S)—N-[(7S,13S,19P)-20-{2-[(1S)-1-methoxyethyl]-5-{4-methyl-4,7-diazaspiro[2.5]octan-7-yl}pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide and (1R,2R,3S)—N-[(7S,13S,19M)-20-{2-[(1S)-1-methoxyethyl]-5-{4-methyl-4,7-diazaspiro[2.5]octan-7-yl}pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

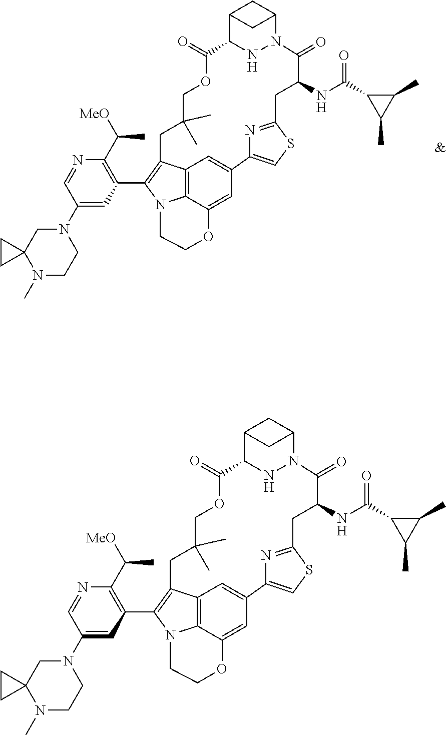

### Step 1: tert-butyl 7-(5-{3-[3-(acetyloxy)-2,2-dimethylpropyl]-6-bromo-1-azatricyclo[6.3.1.0^{4,12}]dodeca-2,4(12),5,7-tetraen-2-yl}-6-[(1S)-1-methoxyethyl]pyridin-3-yl)-4,7-diazaspiro[2.5]octane-4-carboxylate

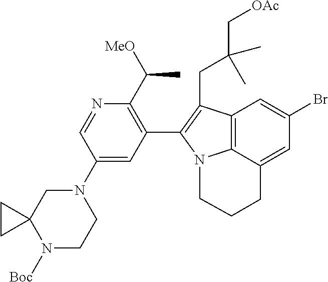 [SMILES: CO[C@@H](C)c1ncc(N2CCN(C(=O)OC(C)(C)C)C3(CC3)C2)cc1-c1c(CC(C)(C)COC(C)=O)c2cc(Br)cc3c2n1CCC3]

The title compound (260 mg, 18%) was prepared in a manner similar to that in example 9 step 2 from (5-{3-[3-(acetyloxy)-2,2-dimethylpropyl]-6-bromo-1-azatricyclo[6.3.1.0²]dodeca-2,4(12),5,7-tetraen-2-yl}-6-[(1S)-1-methoxyethyl]pyridin-3-yl)boronic acid and tert-butyl 4,7-diazaspiro[2.5]octane-4-carboxylate. LC-MS (M+H)⁺=711.3/709.3.

### Step 2: 3-[6-bromo-2-(5-{4,7-diazaspiro[2.5]octan-7-yl}-2-[(1S)-1-methoxyethyl]pyridin-3-yl)-1-azatricyclo[6.3.1.0^{4,12}]dodeca-2,4(12),5,7-tetraen-3-yl]-2,2-dimethylpropan-1-ol

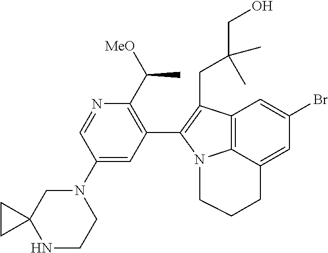

The title compound (220 mg, 100%) was prepared in a manner similar to that in example 32 step 5 from tert-butyl 7-(5-{3-[3-(acetyloxy)-2,2-dimethylpropyl]-6-bromo-1-azatricyclo[6.3.1.0^{4,12}]dodeca-2,4(12),5,7-tetraen-2-yl}-6-[(1S)-1-methoxyethyl]pyridin-3-yl)-4,7-diazaspiro[2.5]octane-4-carboxylate. LC-MS (M+H)⁺=569.3/567.3.

### Step 3: 3-(6-bromo-2-{2-[(1S)-1-methoxyethyl]-5-{4-methyl-4,7-diazaspiro[2.5]octan-7-yl}pyridin-3-yl}-1-azatricyclo[6.3.1.0^{4,12}]dodeca-2,4(12),5,7-tetraen-3-yl)-2,2-dimethylpropan-1-ol

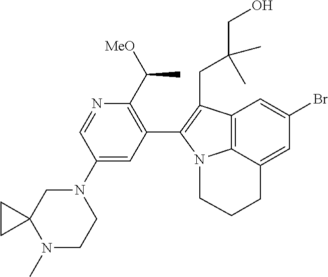 [SMILES: CO[C@@H](C)c1ncc(N2CCN(C)C3(CC3)C2)cc1-c1c(CC(C)(C)CO)c2cc(Br)cc3c2n1CCC3]

The title compound (180 mg, 100%) was prepared in a manner similar to that in example 32 step 6 from 3-[6-bromo-2-(5-{4,7-diazaspiro[2.5]octan-7-yl}-2-[(1S)-1-methoxyethyl]pyridin-3-yl)-1-azatricyclo[6.3.1.0^{4,12}]dodeca-2,4(12),5,7-tetraen-3-yl]-2,2-dimethylpropan-1-ol. LC-MS (M+H)⁺=583.3/581.3.

### Step 4: methyl (4S)-2-[(2S)-2-{[(tert-butoxy)carbonyl]amino}-3-{4-[3-(3-hydroxy-2,2-dimethylpropyl)-2-{2-[(1S)-1-methoxyethyl]-5-{4-methyl-4,7-diazaspiro[2.5]octan-7-yl}pyridin-3-yl}-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylate

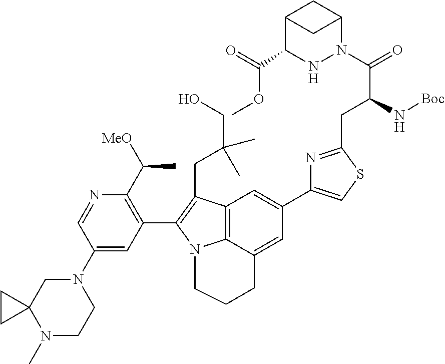 [SMILES: COC(=O)[C@H]1NN(C(=O)[C@H](Cc2nc(-c3cc4c5c(c3)c(CC(C)(C)CO)c(-c3cc(N6CCN(C)C7(CC7)C6)cnc3[C@H](C)OC)n5CCC4)cs2)NC(=O)OC(C)(C)C)C2CC1C2]

The title compound (150 mg, 53%) was prepared in a manner similar to that in example 9 step 4 from 3-(6-bromo-2-{2-[(1S)-1-methoxyethyl]-5-{4-methyl-4,7-diazaspiro[2.5]octan-7-yl}pyridin-3-yl}-1-azatricyclo[6.3.1.0^{4,12}]dodeca-2,4(12),5,7-tetraen-3-yl)-2,2-dimethylpropan-1-ol and methyl (4S)-2-[(2S)-2-{[(tert-butoxy)carbonyl]amino}-3-[4-(4,4,5,5-tetramethyl-1,3,2-dioxaborolan-2-yl)-1,3-thiazol-2-yl]propanoyl]-2,3-diazabicyclo[3.1.1]heptane-4-carboxylate. LC-MS (M+H)⁺=911.3.

### Step 5: (4S)-2-[(2S)-2-{[(tert-butoxy)carbonyl]amino}-3-{4-[3-(3-hydroxy-2,2-dimethylpropyl)-2-{2-[(1S)-1-methoxyethyl]-5-{4-methyl-4,7-diazaspiro[2.5]octan-7-yl}pyridin-3-yl}-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic Acid

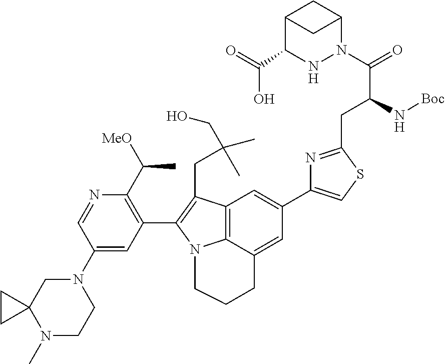 [SMILES: CO[C@@H](C)c1ncc(N2CCN(C)C3(CC3)C2)cc1-c1c(CC(C)(C)CO)c2cc(-c3csc(C[C@H](NC(=O)OC(C)(C)C)C(=O)N4N[C@H](C(=O)O)C5CC4C5)n3)cc3c2n1CCC3]

The title compound (140 mg, 95%) was prepared in a manner similar to that in example 9 step 5 from methyl (4S)-2-[(2S)-2-{[(tert-butoxy)carbonyl]amino}-3-{4-[3-(3-hydroxy-2,2-dimethylpropyl)-2-{2-[(1S)-1-methoxyethyl]-5-{4-methyl-4,7-diazaspiro[2.5]octan-7-yl}pyridin-3-yl}-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylate. LC-MS (M+H)⁺=897.3.

### Step 6: tert-butyl N-[(7S,13S)-20-{2-[(1S)-1-methoxyethyl]-5-{4-methyl-4,7-diazaspiro[2.5]octan-7-yl}pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate

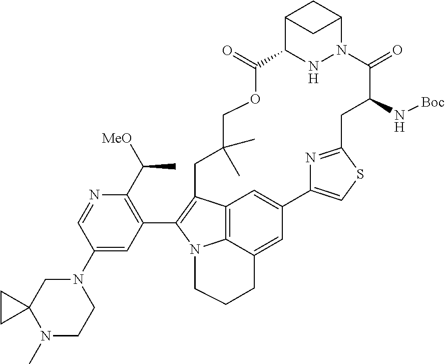 [SMILES: CO[C@@H](C)c1ncc(N2CCN(C)C3(CC3)C2)cc1-c1c2c3cc(cc4c3n1CCC4)-c1csc(n1)C[C@H](NC(=O)OC(C)(C)C)C(=O)N1N[C@H](C(=O)OCC(C)(C)C2)C2CC1C2]

The title compound (100 mg, 68%) was prepared in a manner similar to that in example 9 step 6 from (4S)-2-[(2S)-2-{[(tert-butoxy)carbonyl]amino}-3-{4-[3-(3-hydroxy-2,2-dimethylpropyl)-2-{2-[(1S)-1-methoxyethyl]-5-{4-methyl-4,7-diazaspiro[2.5]octan-7-yl}pyridin-3-yl}-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic acid. LC-MS (M+H)⁺=879.3

### Step 7: (7S,13S)-7-amino-20-{2-[(1S)-1-methoxyethyl]-5-{4-methyl-4,7-diazaspiro[2.5]octan-7-yl}pyridin-3-yl}-17,17-dimethyl-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione

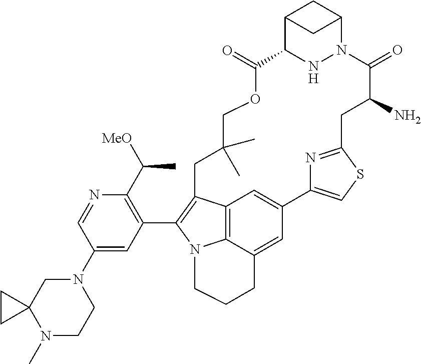 [SMILES: CO[C@@H](C)c1ncc(N2CCN(C)C3(CC3)C2)cc1-c1c2c3cc(cc4c3n1CCC4)-c1csc(n1)C[C@H](N)C(=O)N1N[C@H](C(=O)OCC(C)(C)C2)C2CC1C2]

The title compound (100 mg, 75%) was prepared in a manner similar to that in example 9 step 7 from tert-butyl N-[(7S,13S)-20-{2-[(1S)-1-methoxyethyl]-5-{4-methyl-4,7-diazaspiro[2.5]octan-7-yl}pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate. LC-MS (M+H)⁺=779.3

### Step 8: (1R,2R,3S)—N-[(7S,13S,19P)-20-{2-[(1S)-1-methoxyethyl]-5-{4-methyl-4,7-diazaspiro[2.5]octan-7-yl}pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide and (1R,2R,3S)—N-[(7S,13S,19M)-20-{2-[(1S)-1-methoxyethyl]-5-{4-methyl-4,7-diazaspiro[2.5]octan-7-yl}pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

Atrop-34 (P-isomer, 13 mg, 11%) and example 34 (M-isomer, 10 mg, 9%) were prepared in a manner similar to that in example 9 step 8 from (7S,13S)-7-amino-20-{2-[(1S)-1-methoxyethyl]-5-{4-methyl-4,7-diazaspiro[2.5]octan-7-yl}pyridin-3-yl}-17,17-dimethyl-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione and (1r,2R,3S)-2,3-dimethylcyclopropane-1-carboxylic acid.

Atrop-34: ¹H NMR (400 MHz, DMSO-d6) δ 8.40 (d, J=8.8 Hz, 1H), 8.37 (d, J=1.9 Hz, 1H), 8.23 (s, 1H), 7.70 (s, 1H), 7.40 (s, 1H), 7.32 (d, J=1.9 Hz, 1H), 5.86-5.84 (m, 1H), 5.28 (t, J=8.2 Hz, 1H), 4.73-4.70 (m, 1H), 4.47 (d, J=4.6 Hz, 1H), 3.93-3.88 (m, 1H), 3.85 (brs, 1H), 3.61-3.58 (m, 1H), 3.52-3.49 (m, 1H), 3.47-3.38 (m, 1H), 3.27-3.21 (m, 2H), 3.18-3.15 (m, 1H), 3.13-3.11 (m, 1H), 3.09-3.07 (m, 1H), 3.05 (s, 3H), 3.02-2.99 (m, 2H), 2.95-2.93 (m, 2H), 2.91-2.89 (m, 2H), 2.65-2.63 (m, 1H), 2.38-2.28 (m, 2H), 2.25 (s, 3H), 2.21-2.07 (m, 2H), 1.97 (s, 1H), 1.61-1.57 (m, 1H), 1.26-1.17 (m, 1H), 1.15-1.13 (m, 2H), 1.11-1.01 (m, 9H), 0.91 (s, 3H), 0.59 (s, 2H), 0.49-0.47 (m, 4H), 0.42 (s, 1H). LC-MS (M+H)⁺=875.4.

Example 34: ¹H NMR (400 MHz, DMSO-d6) δ 8.37-8.35 (m, 2H), 8.20 (s, 1H), 7.67 (s, 1H), 7.39 (s, 1H), 7.08 (d, J=2.4 Hz, 1H), 5.89-5.86 (m, 1H), 5.33 (brs, 1H), 4.64-4.61 (m, 1H), 4.45-4.44 (m, 1H), 4.23-4.20 (m, 1H), 4.17-4.14 (m, 1H), 3.58-3.55 (m, 1H), 3.53-3.42 (m, 2H), 3.22-3.17 (m, 3H), 3.16 (s, 3H), 3.12-3.06 (m, 1H), 3.04 (s, 2H), 2.96 (brs, 1H), 2.91-2.84 (m, 3H), 2.82 (s, 1H), 2.62 (s, 1H), 2.59-2.58 (m, 1H), 2.28 (s, 1H), 2.24 (s, 3H), 2.14-2.04 (m, 2H), 1.99-1.92 (m, 1H), 1.53-1.48 (m, 1H), 1.41 (s, 1H), 1.29 (d, J=6.0 Hz, 3H), 1.12-1.11 (m, 2H), 1.05-1.01 (m, 6H), 0.87-0.77 (m, 4H), 0.56 (s, 2H), 0.44 (s, 2H), 0.36 (s, 3H). LC-MS (M+H)⁺=875.4.

Atrop-35 & Example 35: (1R,2R,3S)—N-[(7S,13S,19P)-20-{2-[(1S)-1-methoxyethyl]-5-(6-oxa-2-azaspiro[3.4]octan-2-yl)pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide and (1R,2R,3S)—N-[(7S,13S,19M)-20-{2-[(1S)-1-methoxyethyl]-5-(6-oxa-2-azaspiro[3.4]octan-2-yl)pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

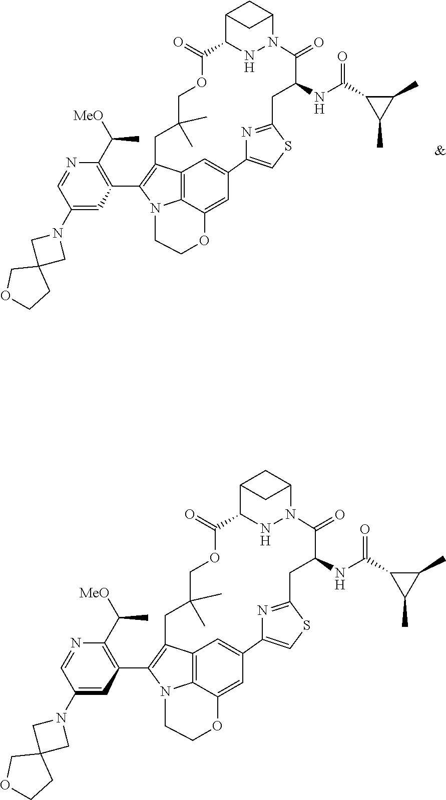

### Step 1: 3-(6-bromo-2-{2-[(1S)-1-methoxyethyl]-5-{6-oxa-2-azaspiro[3.4]octan-2-yl}pyridin-3-yl}-9-oxa-1-azatricyclo[6.3.1.0^{4,12}]dodeca-2,4(12),5,7-tetraen-3-yl)-2,2-dimethylpropyl Acetate

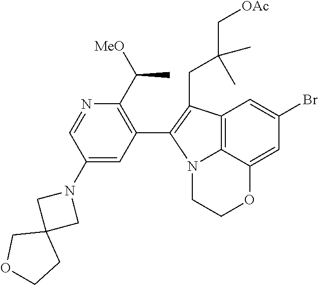 [SMILES: CO[C@@H](C)c1ncc(N2CC3(CCOC3)C2)cc1-c1c(CC(C)(C)COC(C)=O)c2cc(Br)cc3c2n1CCO3]

The title compound (1.29 g, 72%) was prepared in a manner similar to that in example 9 step 2 from (5-{3-[3-(acetyloxy)-2,2-dimethylpropyl]-6-bromo-9-oxa-1-azatricyclo[6.3.1.0^{4,12}]dodeca-2,4(12),5,7-tetraen-2-yl}-6-[(1S)-1-methoxyethyl]pyridin-3-yl)boronic acid and 6-oxa-2-azaspiro[3.4]octane. LC-MS (M+H)⁺=612.4/614.4.

### Step 2: 3-(6-bromo-2-{2-[(1S)-1-methoxyethyl]-5-{6-oxa-2-azaspiro[3.4]octan-2-yl}pyridin-3-yl}-9-oxa-1-azatricyclo[6.3.1.0^{4,12}]dodeca-2,4(12),5,7-tetraen-3-yl)-2,2-dimethylpropan-1-ol

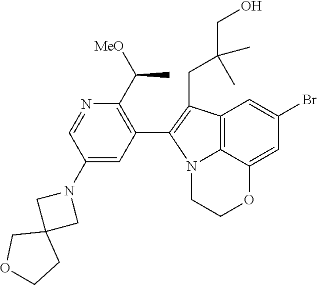 [SMILES: CO[C@@H](C)c1ncc(N2CC3(CCOC3)C2)cc1-c1c(CC(C)(C)CO)c2cc(Br)cc3c2n1CCO3]

To a solution of 3-(6-bromo-2-(2-[(1S)-1-methoxyethyl]-5-(6-oxa-2-azaspiro[3.4]octan-2-yl)pyridin-3-yl)-9-oxa-1-azatricyclo[6.3.1.0^{4,12}]dodeca-2,4(12),5,7-tetraen-3-yl)-2,2-dimethylpropyl acetate (1.29 g, 2.11 mmol) in THF (8 mL) and water (4 mL) was added LiOH (0.89 g, 21.1 mmol). The resulting mixture was stirred overnight at room temperature. The mixture was diluted with water (30 mL) and acidified to pH ˜5 with 0.5 N hydrochloric acid at 0° C. The mixture was extracted with EA (3×40 mL). The organic phases were combined, dried over Na₂SO₄, filtered and concentrated under vacuum to give the title compound (1.0 g, 83%). LC-MS (M+H)⁺=570.2/572.2.

### Step 3: [3-(3-hydroxy-2,2-dimethylpropyl)-2-{2-[(1S)-1-methoxyethyl]-5-{6-oxa-2-azaspiro[3.4]octan-2-yl}pyridin-3-yl}-9-oxa-1-azatricyclo[6.3.1.0^{4,12}]dodeca-2,4(12),5,7-tetraen-6-yl]boronic Acid

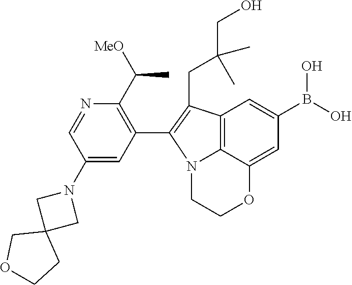 [SMILES: CO[C@@H](C)c1ncc(N2CC3(CCOC3)C2)cc1-c1c(CC(C)(C)CO)c2cc(B(O)O)cc3c2n1CCO3]

To a mixture of 3-(6-bromo-2-{2-[(1S)-1-methoxyethyl]-5-{6-oxa-2-azaspiro[3.4]octan-2-yl}pyridin-3-yl}-9-oxa-1-azatricyclo[6.3.1.0^{4,12}]dodeca-2,4(12),5,7-tetraen-3-yl)-2,2-dimethylpropan-1-ol (1 g, 1.76 mmol), KOPiv (0.55 g, 3.87 mmol), B₂(OH)₄ (0.315 g, 3.51 mmol), XPhos (0.167 g, 0.351 mmol) and XPhos-Pd-G3 (0.149 g, 0.176 mmol) was added 2-MeTHF (9 mL) and MeOH (3 mL). The mixture was stirred for 1 h at 30° C. under nitrogen. The mixture was cooled to room temperature and diluted with EA (50 mL), washed with brine (40 mL). The organic phase was dried over Na₂SO₄, filtered and concentrated under vacuum. The residue was purified by silica gel chromatograph (DCM:MeOH=20:1) to give the title compound (0.6 g, 63%). LC-MS (M+H)⁺=536.4.

### Step 4: methyl (4S)-2-[(2S)-2-{[(1R,2R,3S)-2,3-dimethylcyclopropyl]formamido}-3-{4-[3-(3-hydroxy-2,2-dimethylpropyl)-2-{2-[(1S)-1-methoxyethyl]-5-{6-oxa-2-azaspiro[3.4]octan-2-yl}pyridin-3-yl}-9-oxa-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylate

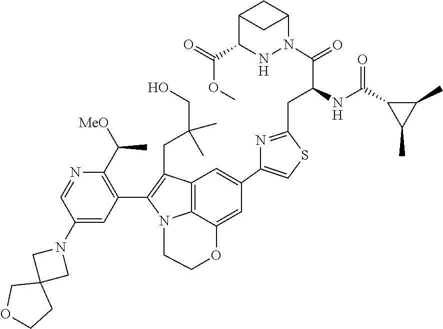 [SMILES: COC(=O)[C@H]1NN(C(=O)[C@H](Cc2nc(-c3cc4c5c(c3)c(CC(C)(C)CO)c(-c3cc(N6CC7(CCOC7)C6)cnc3[C@H](C)OC)n5CCO4)cs2)NC(=O)[C@H]2[C@H](C)[C@@H]2C)C2CC1C2]

To a mixture of [3-(3-hydroxy-2,2-dimethylpropyl)-2-{2-[(1S)-1-methoxyethyl]-5-{6-oxa-2-azaspiro[3.4]octan-2-yl}pyridin-3-yl}-9-oxa-1-azatricyclo[6.3.1.0^{4,12}]dodeca-2,4(12),5,7-tetraen-6-yl]boronic acid (0.3 g, 0.56 mmol) and methyl (4S)-2-[(2S)-3-(4-bromo-1,3-thiazol-2-yl)-2-{[(1R,2R,3S)-2,3-dimethylcyclopropyl]formamido}propanoyl]-2,3-diazabicyclo[3.1.1]heptane-4-carboxylate (0.27 g, 0.56 mmol) in dioxane (5 mL) and water (0.5 mL) was added K₃PO₄ (0.237 g, 1.12 mmol) and Pd(dtbpf)Cl₂ (36.5 mg, 0.056 mmol). The mixture was stirred for 3 h at 75° C. under nitrogen and then cooled to room temperature. The mixture was diluted with EA (50 mL), successively washed with water (50 mL), brine (50 mL), dried over Na₂SO₄, filtered and concentrated under vacuum. The residue was purified by silica gel chromatograph (DCM:MeOH=20:1) to give the title compound (0.3 g, 60%). LC-MS (M+H)⁺=896.5.

### Step 5: (4S)-2-{[(2S)-2-{[(1R,2R,3S)-2,3-dimethylcyclopropyl]formamido}-3-{4-[3-(3-hydroxy-2,2-dimethylpropyl)-2-{2-[(1S)-1-methoxyethyl]-5-{6-oxa-2-azaspiro[3.4]octan-2-yl}pyridin-3-yl}-9-oxa-1-azatricyclo[6.3.1.0^{4,12}]dodeca-2,4(12),5,7-tetraen-6-yl}-1,3-thiazol-2-yl}propanoyl]-2,3-diazabicyclo[3.1.1]heptane-4-carboxylic Acid

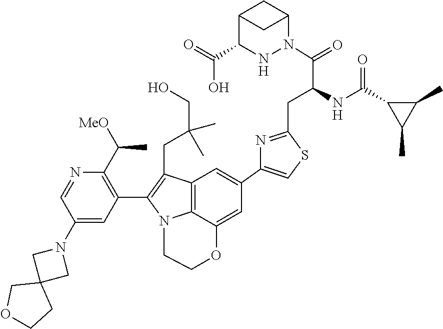 [SMILES: CO[C@@H](C)c1ncc(N2CC3(CCOC3)C2)cc1-c1c(CC(C)(C)CO)c2cc(-c3csc(C[C@H](NC(=O)[C@H]4[C@H](C)[C@@H]4C)C(=O)N4N[C@H](C(=O)O)C5CC4C5)n3)cc3c2n1CCO3]

To a solution of methyl (4S)-2-[(2S)-2-{[(R,2R,3S)-2,3-dimethylcyclopropyl]formamido}-3-{4-[3-(3-hydroxy-2,2-dimethylpropyl)-2-{2-[(1S)-1-methoxyethyl]-5-{6-oxa-2-azaspiro[3.4]octan-2-yl}pyridin-3-yl}-9-oxa-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylate (0.3 g, 0.335 mmol) in THF (6 mL) was added LiOH·H₂O (42 mg, 1.0 mmol) in water (4 mL). The resulting mixture was stirred for 1 h at room temperature. The mixture was diluted with water (10 mL) and extracted with MTBE (20 mL×2). The aqueous phase was acidified to pH ˜7 with 0.5 N hydrochloric acid at 0° C. The mixture was extracted with DCM (30 mL×2). The combined organic layer was dried over Na₂SO₄, filtered and concentrated under vacuum to give the title compound (0.28 g, 95%). LC-MS (M+H)⁺=882.5

### Step 6: (1R,2R,3S)—N-[(7S,13S,19P)-20-{2-[(1S)-1-methoxyethyl]-5-{6-oxa-2-azaspiro[3.4]octan-2-yl}pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide and (1R,2R,3S)—N-[(7S,13S,19M)-20-{2-[(1S)-1-methoxyethyl]-5-{6-oxa-2-azaspiro[3.4]octan-2-yl}pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

To a solution of (4S)-2-[(2S)-2-{[(R,2R,3S)-2,3-dimethylcyclopropyl]formamido}-3-{4-[3-(3-hydroxy-2,2-dimethylpropyl)-2-{2-[(1S)-1-methoxyethyl]-5-{6-oxa-2-azaspiro[3.4]octan-2-yl}pyridin-3-yl}-9-oxa-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic acid (0.28 g, 0.32 mmol), DIPEA (86 mg, 0.67 mmol), DMAP (20 mg, 0.052 mmol) in DCM (30 mL) was added EDCI (0.193 g, 1 mmol) and HOBt (90 mg, 0.67 mmol). The mixture was stirred for 16 h at room temperature and then diluted with EA (40 mL), washed with brine (3×20 mL). The organic phase was dried over Na₂SO₄, filtered, and concentrated under vacuum. The residue was purified by prep-TLC (DCM:MeOH=10:1) to give a pair of atropisomers. The atropisomers were further separated by prep-HPLC to give Atrop-35 (P-isomer, 14 mg, 5%) and example 35 (M-isomer, 12 mg, 4.4%).

Atrop-35: ¹H NMR (400 MHz, DMSO-d6) δ 8.42 (d, J=8.9 Hz, 1H), 8.07 (s, 1H), 8.00 (s, 1H), 7.77 (s, 1H), 7.16 (s, 1H), 6.93 (s, 1H), 5.87 (d, J=11.0 Hz, 1H), 5.31 (t, J=7.9 Hz, 1H), 4.74 (d, J=10.8 Hz, 1H), 4.56-4.41 (m, 3H), 4.08-3.97 (m, 2H), 3.964-3.91 (m, 2H), 3.84-3.82 (m, 4H), 3.73 (t, J=6.9 Hz, 2H), 3.69-3.59 (m, 2H), 3.53 (d, J=10.9 Hz, 1H), 3.21-3.25 (m, 2H), 3.10 (s, 3H), 3.00 (d, J=14.2 Hz, 1H), 2.70-2.66 (m, 1H), 2.37-2.31 (m, 2H), 2.18-2.12 (m, 3H), 1.62 (t, J=9.4 Hz, 1H), 1.28-1.21 (m, 1H), 1.20-1.13 (m, 5H), 1.11-1.06 (m, 7H), 0.96 (s, 3H), 0.53 (s, 3H).

LC-MS (M+H)⁺=864.6

Example 35: ¹H NMR ¹H NMR (400 MHz, DMSO-d6) δ 8.42 (d, J=8.9 Hz, 1H), 8.04 (s, 1H), 7.97 (d, J=2.6 Hz, 1H), 7.78 (s, 1H), 7.16 (s, 1H), 6.71 (d, J=2.6 Hz, 1H), 5.90 (d, J=11.1 Hz, 1H), 5.33 (t, J=7.9 Hz, 1H), 4.72 (d, J=11.0 Hz, 1H), 4.68-4.45 (m, 3H), 4.31 (d, J=6.4 Hz, 2H), 3.92-3.88 (m, 4H), 3.82 (s, 2H), 3.75-3.66 (m, 3H), 3.57 (dd, J=21.8, 10.8 Hz, 2H), 3.26-3.22 (m, 4H), 3.14-3.09 (m, 1H), 2.92 (d, J=14.2 Hz, 1H), 2.70-2.61 (m, 2H), 2.37-2.31 (m, 1H), 2.23-2.10 (m, 3H), 1.60 (t, J=9.3 Hz, 1H), 1.33 (d, J=6.1 Hz, 3H), 1.28-1.13 (m, 4H), 1.10-1.06 (m, 6H), 0.94 (s, 3H), 0.38 (s, 3H). LC-MS (M+H)⁺=864.6

### Example 36: (1R,2R,3S)—N-[(7S,13S,19M)-20-{5-[(3R,5S)-4-ethyl-3,5-dimethylpiperazin-1-yl]-2-[(1S)-1-methoxyethyl]pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

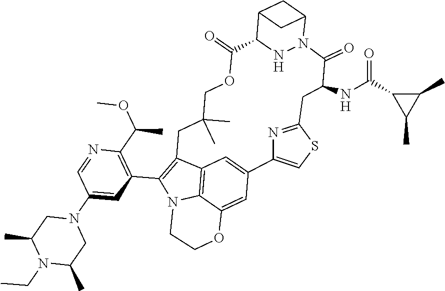

### Step 1: tert-butyl (2R,6S)-4-(5-{3-[3-(acetyloxy)-2,2-dimethylpropyl]-6-bromo-9-oxa-1-azatricyclo[6.3.1.0^{4,12}]dodeca-2,4(12),5,7-tetraen-2-yl}-6-[(1S)-1-methoxyethyl]pyridin-3-yl)-2,6-dimethylpiperazine-1-carboxylate

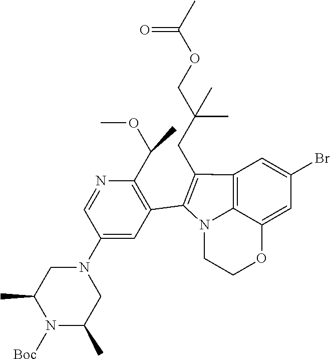 [SMILES: CO[C@@H](C)c1ncc(N2C[C@@H](C)N(C(=O)OC(C)(C)C)[C@@H](C)C2)cc1-c1c(CC(C)(C)COC(C)=O)c2cc(Br)cc3c2n1CCO3]

The title compound (360 mg, 31%) was prepared in a manner similar to that in example 9 step 2 from 3-(6-bromo-2-{2-[(1S)-1-methoxyethyl]-5-(4,4,5,5-tetramethyl-1,3,2-dioxaborolan-2-yl)pyridin-3-yl}-9-oxa-1-azatricyclo[6.3.1.0^{4,12}]dodeca-2,4(12),5,7-tetraen-3-yl)-2,2-dimethylpropyl acetate and tert-butyl (2R,6S)-2,6-dimethylpiperazine-1-carboxylate. LC-MS (M+H)⁺=713.2/715.2.

### Step 2: 3-(6-bromo-2-{5-[(3R,5S)-4-ethyl-3,5-dimethylpiperazin-1-yl]-2-[(1S)-1-methoxyethyl]pyridin-3-yl}-9-oxa-1-azatricyclo[6.3.1.0^{4,12}]dodeca-2,4(12),5,7-tetraen-3-yl)-2,2-dimethylpropyl Acetate

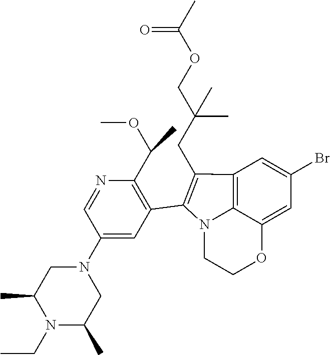 [SMILES: CCN1[C@H](C)CN(c2cnc([C@H](C)OC)c(-c3c(CC(C)(C)COC(C)=O)c4cc(Br)cc5c4n3CCO5)c2)C[C@@H]1C]

To a round-bottom flask charged with tert-butyl (2R,6S)-4-(5-{3-[3-(acetyloxy)-2,2-dimethylpropyl]-6-bromo-9-oxa-1-azatricyclo[6.3.1.0^{4,12}]dodeca-2,4(12),5,7-tetraen-2-yl}-6-[(1S)-1-methoxyethyl]pyridin-3-yl)-2,6-dimethylpiperazine-1-carboxylate (360 mg, 0.5 mmol) was added HCl/dioxane (10 mL, 4 M). The reaction mixture was stirred at room temperature for 2 h. Solvents was removed in vacuo to give a white solid. The solid was dissolved in DMF (5 mL), and then iodoethane (94 mg, 0.6 mmol) and K₂CO₃ (139 mg, 1 mmol) were added. The reaction mixture was heated 80° C. and stirred for 12 h. The reaction mixture was cooled to room temperature and diluted with water (20 mL), extracted with EA (3×10 mL). The combined organic layer washed with brine (10 mL), dried over Na₂SO₄, filtered and evaporated in vacuo to give a residue. The residue was purified by prep-TLC (DCM/MeOH=15:1) to give the title compound (130 mg, 40%). LC-MS (M+H)⁺=641.2/643.2.

### Step 3: 3-(6-bromo-2-{5-[(3R,5S)-4-ethyl-3,5-dimethylpiperazin-1-yl]-2-[(1S)-1-methoxyethyl]pyridin-3-yl}-9-oxa-1-azatricyclo[6.3.1.0^{4,12}]dodeca-2,4(12),5,7-tetraen-3-yl)-2,2-dimethylpropan-1-ol

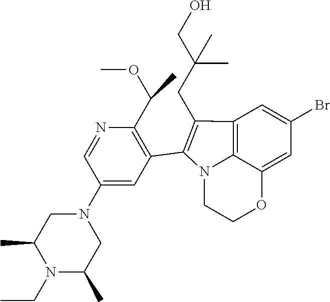 [SMILES: CCN1[C@H](C)CN(c2cnc([C@H](C)OC)c(-c3c(CC(C)(C)CO)c4cc(Br)cc5c4n3CCO5)c2)C[C@@H]1C]

The title compound (120 mg, 80%) was prepared in a manner similar to that in example 35 step 2 from 3-(6-bromo-2-{5-[(3R,5S)-4-ethyl-3,5-dimethylpiperazin-1-yl]-2-[(1S)-1-methoxyethyl]pyridin-3-yl}-9-oxa-1-azatricyclo[6.3.1.0^{4,12}]dodeca-2,4(12),5,7-tetraen-3-yl)-2,2-dimethylpropyl acetate. LC-MS (M+H)⁺=599.2/601.2.

### Step 4: (2-{5-[(3R,5S)-4-ethyl-3,5-dimethylpiperazin-1-yl]-2-[(1S)-1-methoxyethyl]pyridin-3-yl}-3-(3-hydroxy-2,2-dimethylpropyl)-9-oxa-1-azatricyclo[6.3.1.0^{4,12}]dodeca-2,4(12),5,7-tetraen-6-yl)boronic Acid

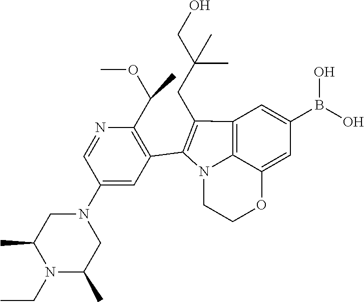 [SMILES: CCN1[C@H](C)CN(c2cnc([C@H](C)OC)c(-c3c(CC(C)(C)CO)c4cc(B(O)O)cc5c4n3CCO5)c2)C[C@@H]1C]

The title compound (90 mg, 80%) was prepared in a manner similar to that in example 35 step 3 from 3-(6-bromo-2-{5-[(3R,5S)-4-ethyl-3,5-dimethylpiperazin-1-yl]-2-[(1S)-1-methoxyethyl]pyridin-3-yl}-9-oxa-1-azatricyclo[6.3.1.0^{4,12}]dodeca-2,4(12),5,7-tetraen-3-yl)-2,2-dimethylpropan-1-ol. LC-MS (M+H)⁺=565.3.

### Step 5: (4S)-2-[(2S)-2-{[(1R,2R,3S)-2,3-dimethylcyclopropyl]formamido}-3-[4-(2-{5-[(3R,5S)-4-ethyl-3,5-dimethylpiperazin-1-yl]-2-[(1S)-1-methoxyethyl]pyridin-3-yl}-3-(3-hydroxy-2,2-dimethylpropyl)-9-oxa-1-azatricyclo[6.3.1.0^{4,12}]dodeca-2,4(12),5,7-tetraen-6-yl)-1,3-thiazol-2-yl]propanoyl]-2,3-diazabicyclo[3.1.1]heptane-4-carboxylic Acid

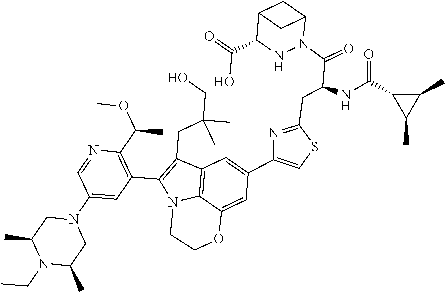 [SMILES: CCN1[C@H](C)CN(c2cnc([C@H](C)OC)c(-c3c(CC(C)(C)CO)c4cc(-c5csc(C[C@H](NC(=O)[C@H]6[C@H](C)[C@@H]6C)C(=O)N6N[C@H](C(=O)O)C7CC6C7)n5)cc5c4n3CCO5)c2)C[C@@H]1C]

The title compound (71 mg, 48%) was prepared in a manner similar to that in example 32 step 8 from (2-{5-[(3R,5S)-4-ethyl-3,5-dimethylpiperazin-1-yl]-2-[(1S)-1-methoxyethyl]pyridin-3-yl}-3-(3-hydroxy-2,2-dimethylpropyl)-9-oxa-1-azatricyclo[6.3.1.0^{4,12}]dodeca-2,4(12),5,7-tetraen-6-yl)boronic acid and (4S)-2-[(2S)-3-(4-bromo-1,3-thiazol-2-yl)-2-{[(R,2R,3S)-2,3-dimethylcyclopropyl]formamido}propanoyl]-2,3-diazabicyclo[3.1.1]heptane-4-carboxylic acid. LC-MS (M+H)⁺=911.4.

### Step 6: (1R,2R,3S)—N-[(7S,13S,19M)-20-{5-[(3R,5S)-4-ethyl-3,5-dimethylpiperazin-1-yl]-2-[(1S)-1-methoxyethyl]pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

Example 36 (10 mg, 15%) was prepared in a manner similar to that in example 32 step 9 from (4S)-2-[(2S)-2-{[(R,2R,3S)-2,3-dimethylcyclopropyl]formamido}-3-[4-(2-{5-[(3R,5S)-4-ethyl-3,5-dimethylpiperazin-1-yl]-2-[(1S)-1-methoxyethyl]pyridin-3-yl}-3-(3-hydroxy-2,2-dimethylpropyl)-9-oxa-1-azatricyclo[6.3.1.0^{4,12}]dodeca-2,4(12),5,7-tetraen-6-yl)-1,3-thiazol-2-yl]propanoyl]-2,3-diazabicyclo[3.1.1]heptane-4-carboxylic acid. ¹H NMR (399 MHz, DMSO) δ 8.41-8.34 (m, 2H), 8.00 (s, 1H), 7.73 (s, 1H), 7.14 (d, J=1.9 Hz, 1H), 7.11 (s, 1H), 5.86 (d, J=10.9 Hz, 1H), 5.29 (t, J=8.3 Hz, 1H), 4.66 (d, J=11.0 Hz, 1H), 4.60-4.52 (m, 2H), 4.45 (d, J=4.9 Hz, 1H), 4.30-4.23 (m, 2H), 3.69-3.65 (m, 3H), 3.57-3.47 (m, 2H), 3.17 (s, 4H), 3.07 (dd, J=14.5, 7.3 Hz, 1H), 2.87 (d, J=14.6 Hz, 1H), 2.77 (d, J=7.0 Hz, 2H), 2.61 (d, J=8.3 Hz, 4H), 2.38 (d, J=6.2 Hz, 2H), 2.29 (d, J=6.6 Hz, 2H), 2.08 (t, J=10.0 Hz, 1H), 1.54 (t, J=9.3 Hz, 1H), 1.29 (d, J=5.8 Hz, 3H), 1.18 (d, J=6.6 Hz, 2H), 1.12 (s, 2H), 1.05-0.98 (m, 12H), 0.88 (s, 3H), 0.79 (t, J=6.9 Hz, 3H), 0.32 (s, 3H). LC-MS (M+H)⁺=893.4.

### Example 37: (1R,2R,3S)—N-[(7S,13S,19M)-20-{2-[(1S)-1-methoxyethyl]-5-(7-oxa-2-azaspiro[3.5]nonan-2-yl)pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

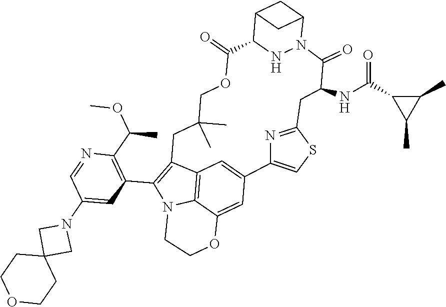

### Step 1: {5-[6-bromo-3-(3-hydroxy-2,2-dimethylpropyl)-9-oxa-1-azatricyclo[6.3.1.0^{4,12}]dodeca-2,4(12),5,7-tetraen-2-yl]-6-[(1S)-1-methoxyethyl]pyridin-3-yl}boronic Acid

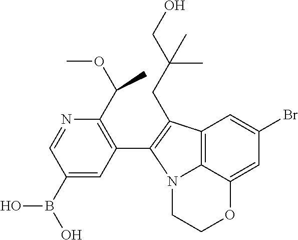 [SMILES: CO[C@@H](C)c1ncc(B(O)O)cc1-c1c(CC(C)(C)CO)c2cc(Br)cc3c2n1CCO3]

To a sealed tube charged with 3-(6-bromo-2-{2-[(1S)-1-methoxyethyl]pyridin-3-yl}-9-oxa-1-azatricyclo[6.3.1.0^{4,12}]dodeca-2,4(12),5,7-tetraen-3-yl)-2,2-dimethylpropan-1-ol (5 g, 10 mmol), dtbpy (536 mg, 2 mmol), and (1,5-cyclooctadiene)(methoxy)iridium(I) dimer (662 mg, 1 mmol) was added THF (100 mL) and pinacolborane (7.7 g, 60 mmol). The reaction mixture was heated to 80° C. for 12 h and then cooled to room temperature. To the reaction mixture was added water (50 mL). The resulting mixture was extracted with EA (100 mL), dried over Na₂SO₄ filtered and concentrated in vacuum. The residue was purified by reverse-phase column chromatography to give the product (4 g, 74%). LC-MS (M+H)⁺=503.1/505.1.

### Step 2: 3-(6-bromo-2-{2-[(1S)-1-methoxyethyl]-5-{7-oxa-2-azaspiro[3.5]nonan-2-yl}pyridin-3-yl}-9-oxa-1-azatricyclo[6.3.1.0^{4,12}]dodeca-2,4(12),5,7-tetraen-3-yl)-2,2-dimethylpropan-1-ol

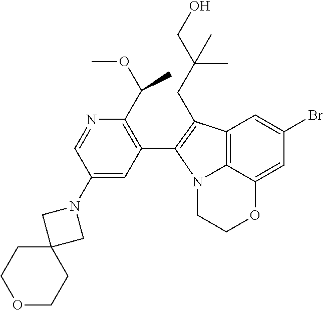 [SMILES: CO[C@@H](C)c1ncc(N2CC3(CCOCC3)C2)cc1-c1c(CC(C)(C)CO)c2cc(Br)cc3c2n1CCO3]

The title compound (270 mg, 29%) was prepared in a manner similar to that in example 9 step 2 from {5-[6-bromo-3-(3-hydroxy-2,2-dimethylpropyl)-9-oxa-1-azatricyclo[6.3.1.0^{4,12}]dodeca-2,4(12),5,7-tetraen-2-yl]-6-[(1S)-1-methoxyethyl]pyridin-3-yl}boronic acid and 7-oxa-2-azaspiro[3.5]nonane. LC-MS (M+H)⁺=584.2/586.2.

### Step 3: [3-(3-hydroxy-2,2-dimethylpropyl)-2-{2-[(1S)-1-methoxyethyl]-5-{7-oxa-2-azaspiro[3.5]nonan-2-yl}pyridin-3-yl}-9-oxa-1-azatricyclo[6.3.1.0^{4,12}]dodeca-2,4(12),5,7-tetraen-6-yl]boronic Acid

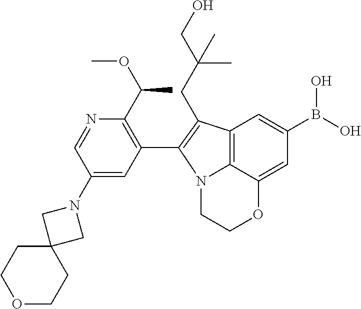 [SMILES: CO[C@@H](C)c1ncc(N2CC3(CCOCC3)C2)cc1-c1c(CC(C)(C)CO)c2cc(B(O)O)cc3c2n1CCO3]

The title compound (190 mg, 80%) was prepared in a manner similar to that in example 32 step 7 from 3-(6-bromo-2-{2-[(1S)-1-methoxyethyl]-5-{7-oxa-2-azaspiro[3.5]nonan-2-yl}pyridin-3-yl}-9-oxa-1-azatricyclo[6.3.1.0^{4,12}]dodeca-2,4(12),5,7-tetraen-3-yl)-2,2-dimethylpropan-1-ol. LC-MS (M+H)⁺=550.3.

### Step 4: (4S)-2-[(2S)-2-{[(1R,2R,3S)-2,3-dimethylcyclopropyl]formamido}-3-{4-[3-(3-hydroxy-2,2-dimethylpropyl)-2-{2-[(1S)-1-methoxyethyl]-5-{7-oxa-2-azaspiro[3.5]nonan-2-yl}pyridin-3-yl}-9-oxa-1-azatricyclo[6.3.1.0^{4,12}]dodeca-2,4(12),5,7-tetraen-6-yl]-1,3-thiazol-2-yl}propanoyl-2,3-diazabicyclo[3.1.1]heptane-4-carboxylic Acid

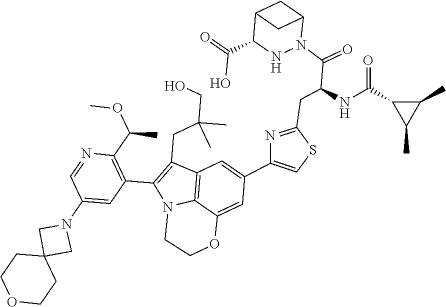 [SMILES: CO[C@@H](C)c1ncc(N2CC3(CCOCC3)C2)cc1-c1c(CC(C)(C)CO)c2cc(-c3csc(C[C@H](NC(=O)[C@H]4[C@H](C)[C@@H]4C)C(=O)N4N[C@H](C(=O)O)C5CC4C5)n3)cc3c2n1CCO3]

The title compound (140 mg, 45%) was prepared in a manner similar to that in example 32 step 8 from [3-(3-hydroxy-2,2-dimethylpropyl)-2-{2-[(1S)-1-methoxyethyl]-5-{7-oxa-2-azaspiro[3.5]nonan-2-yl}pyridin-3-yl}-9-oxa-1-azatricyclo[6.3.1.0^{4,12}]dodeca-2,4(12),5,7-tetraen-6-yl]boronic acid. LC-MS (M+H)⁺=896.4.

### Step 5: (1R,2R,3S)—N-[(7S,13S,19M)-20-{2-[(1S)-1-methoxyethyl]-5-{7-oxa-2-azaspiro[3.5]nonan-2-yl}pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

Example 37 (10 mg, 7%) was prepared in a manner similar to that in example 32 step 9 from (4S)-2-[(2S)-2-{[(R,2R,3S)-2,3-dimethylcyclopropyl]formamido}-3-{4-[3-(3-hydroxy-2,2-dimethylpropyl)-2-{2-[(1S)-1-methoxyethyl]-5-{7-oxa-2-azaspiro[3.5]nonan-2-yl}pyridin-3-yl}-9-oxa-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic acid. ¹H NMR (400 MHz, DMSO-d6) δ8.38 (d, J=8.8 Hz, 1H), 7.99 (s, 1H), 7.91 (d, J=2.2 Hz, 1H), 7.73 (s, 1H), 7.11 (s, 1H), 6.62 (d, J=2.2 Hz, 1H), 5.85 (d, J=11.0 Hz, 1H), 5.28 (t, J=8.0 Hz, 1H), 4.67 (d, J=11.0 Hz, 1H), 4.59-4.49 (m, 2H), 4.45 (d, J=4.6 Hz, 1H), 4.29-4.23 (m, 2H), 3.65 (s, 5H), 3.58-3.48 (m, 6H), 3.17 (s, 4H), 3.09-3.04 (m, 1H), 2.87 (d, J=14.3 Hz, 1H), 2.61 (d, J=6.0 Hz, 1H), 2.40 (s, 1H), 2.30-2.23 (m, 1H), 2.08 (t, J=9.8 Hz, 1H), 1.71 (s, 4H), 1.56 (t, J=9.3 Hz, 1H), 1.28 (d, J=6.0 Hz, 3H), 1.21-1.15 (m, 1H), 1.11 (d, J=3.9 Hz, 2H), 1.03 (dd, J=11.2, 5.6 Hz, 6H), 0.89 (s, 3H), 0.33 (s, 3H). LC-MS (M+H)⁺=878.4.

Atrop-38 & Example 38: (1R,2R,3S)—N-[(7S,13S,19P)-20-{2-[(1S)-1-methoxyethyl]-5-{6-methyl-2,6-diazaspiro[3.4]octan-2-yl}pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide & (1R,2R,3S)—N-[(7S,13S,19M)-20-{2-[(1S)-1-methoxyethyl]-5-{6-methyl-2,6-diazaspiro[3.4]octan-2-yl}pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

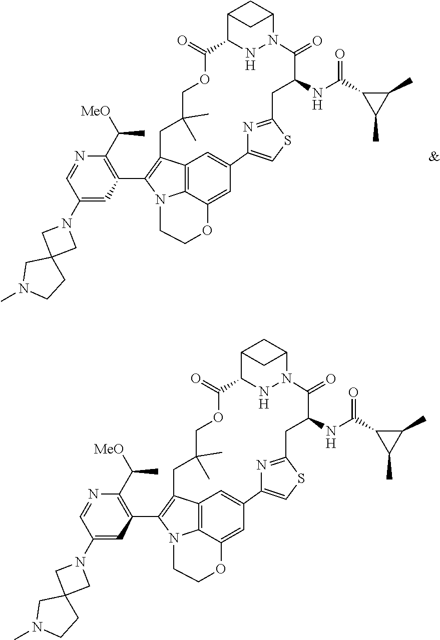

### Step 1: 3-(6-bromo-2-{2-[(1S)-1-methoxyethyl]-5-{6-methyl-2,6-diazaspiro[3.4]octan-2-yl}pyridin-3-yl}-9-oxa-1-azatricyclo[6.3.1.0^{4,12}]dodeca-2,4(12),5,7-tetraen-3-yl)-2,2-dimethylpropyl Acetate

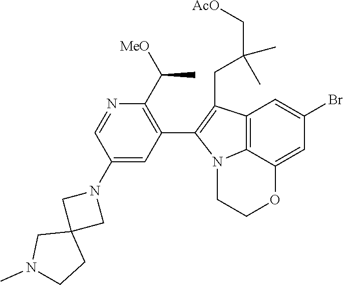 [SMILES: CO[C@@H](C)c1ncc(N2CC3(CCN(C)C3)C2)cc1-c1c(CC(C)(C)COC(C)=O)c2cc(Br)cc3c2n1CCO3]

The title compound (610 mg, 61%) was prepared in a manner similar to that in example 9 step 2 from 3-(6-bromo-2-{2-[(1S)-1-methoxyethyl]-5-(4,4,5,5-tetramethyl-1,3,2-dioxaborolan-2-yl)pyridin-3-yl}-1-azatricyclo[6.3.1.0^{4,12}]dodeca-2,4(12),5,7-tetraen-3-yl)-2,2-dimethylpropyl acetate and 6-methyl-2,6-diazaspiro[3.4]octane. LC-MS (M+H)⁺=625.4.

### Step 2: 3-(6-bromo-2-{2-[(1S)-1-methoxyethyl]-5-{6-methyl-2,6-diazaspiro[3.4]octan-2-yl}pyridin-3-yl}-9-oxa-1-azatricyclo[6.3.1.0^{4,12}]dodeca-2,4(12),5,7-tetraen-3-yl)-2,2-dimethylpropan-1-ol

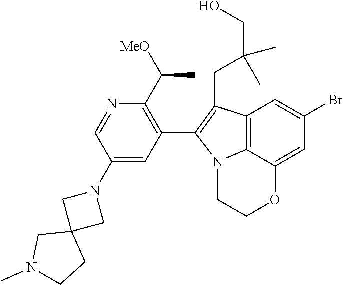 [SMILES: CO[C@@H](C)c1ncc(N2CC3(CCN(C)C3)C2)cc1-c1c(CC(C)(C)CO)c2cc(Br)cc3c2n1CCO3]

To a mixture of 3-(6-bromo-2-{2-[(1S)-1-methoxyethyl]-5-{6-methyl-2,6-diazaspiro[3.4]octan-2-yl}pyridin-3-yl}-9-oxa-1-azatricyclo[6.3.1.0^{4,12}]dodeca-2,4(12),5,7-tetraen-3-yl)-2,2-dimethylpropyl acetate (610 mg, 0.97 mmol) in THF (15 mL) and water (15 mL) was added LiOH (409 mg, 9.7 mmol) and the mixture was stirred at room temperature overnight. The mixture was neutralized with HCl (1 N) until its pH reached 7. The mixture was extracted with EA (10 mL×3). The combined organic layer was washed with brine (10 mL), dried over Na₂SO₄, filtered and concentrated under reduced pressure to give the title compound (540 mg, 95%). LC-MS (M+H)⁺=583.4.

### Step 3: [3-(3-hydroxy-2,2-dimethylpropyl)-2-{2-[(1S)-1-methoxyethyl]-5-{6-methyl-2,6-diazaspiro[3.4]octan-2-yl}pyridin-3-yl}-9-oxa-1-azatricyclo[6.3.1.0^{4,12}]dodeca-2,4(12),5,7-tetraen-6-yl]boronic Acid

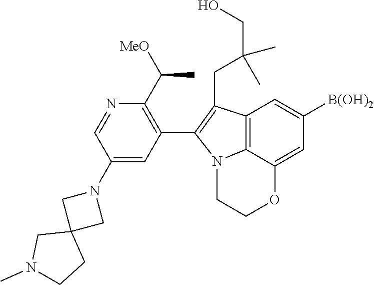 [SMILES: CO[C@@H](C)c1ncc(N2CC3(CCN(C)C3)C2)cc1-c1c(CC(C)(C)CO)c2cc(B(O)O)cc3c2n1CCO3]

The title compound (494 mg, 98%) was prepared in a manner similar to that in example 32 step 7 from 3-(6-bromo-2-{2-[(1S)-1-methoxyethyl]-5-{6-methyl-2,6-diazaspiro[3.4]octan-2-yl}pyridin-3-yl}-9-oxa-1-azatricyclo[6.3.1.0^{4,12}]dodeca-2,4(12),5,7-tetraen-3-yl)-2,2-dimethylpropan-1-ol. LC-MS (M+H)⁺=549.3

### Step 4: (4S)-2-[(2S)-2-{[(1R,2R,3S)-2,3-dimethylcyclopropyl]formamido}-3-{4-[3-(3-hydroxy-2,2-dimethylpropyl)-2-{2-[(1S)-1-methoxyethyl]-5-{6-methyl-2,6-diazaspiro[3.4]octan-2-yl}pyridin-3-yl}-9-oxa-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic Acid

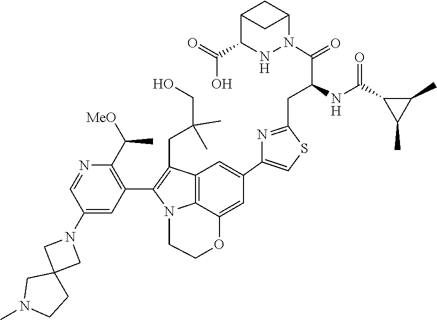

The title compound (90 mg, 11%) was prepared in a manner similar to that in example 32 step 8 from [3-(3-hydroxy-2,2-dimethylpropyl)-2-{2-[(1S)-1-methoxyethyl]-5-{6-methyl-2,6-diazaspiro[3.4]octan-2-yl}pyridin-3-yl}-9-oxa-1-azatricyclo[6.3.1.0^{4,12}]dodeca-2,4(12),5,7-tetraen-6-yl]boronic acid and (4S)-2-[(2S)-3-(4-bromo-1,3-thiazol-2-yl)-2-{[(R,2R,3S)-2,3-dimethylcyclopropyl]formamido}propanoyl]-2,3-diazabicyclo[3.1.1]heptane-4-carboxylic acid. LC-MS (M+H)⁺=895.4.

### Step 5: (1R,2R,3S)—N-[(7S,13S,19P)-20-{2-[(1S)-1-methoxyethyl]-5-{6-methyl-2,6-diazaspiro[3.4]octan-2-yl}pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide & (1R,2R,3S)—N-[(7S,13S,19M)-20-{2-[(1S)-1-methoxyethyl]-5-{6-methyl-2,6-diazaspiro[3.4]octan-2-yl}pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

Atrop-38 (P-isomer, 3 mg, 3%) and example 38 (M-isomer, 14 mg, 16%) was prepared in a manner similar to that in example 32 step 9 from (4S)-2-[(2S)-2-{[(1R,2R,3S)-2,3-dimethylcyclopropyl]formamido}-3-{4-[3-(3-hydroxy-2,2-dimethylpropyl)-2-{2-[(1S)-1-methoxyethyl]-5-{6-methyl-2,6-diazaspiro[3.4]octan-2-yl}pyridin-3-yl}-9-oxa-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic acid.

Atrop-38: ¹H NMR (400 MHz, DMSO) δ 8.43 (d, J=8.7 Hz, 2H), 8.07 (s, 1H), 7.97 (s, 1H), 7.78 (s, 1H), 7.16 (s, 1H), 6.90 (s, 1H), 5.89-5.87 (m, 1H), 5.31 (t, J=8.0 Hz, 1H), 4.75-4.73 (m, 1H), 4.51 (s, 2H), 4.44 (s, 1H), 4.04 (s, 1H), 3.99-3.88 (m, 1H), 3.90-3.88 (m, 1H), 3.85-3.78 (m, 2H), 3.74-3.73 (m, 1H), 3.64-3.61 (m, 2H), 3.54-3.51 (m, 2H), 3.24-3.21 (m, 2H), 3.16-3.14 (m, 1H), 3.10 (s, 3H), 3.07-2.97 (m, 2H), 2.67 (brs, 3H), 2.46-2.45 (m, 1H), 2.35-2.33 (m, 2H), 2.24 (s, 3H), 2.14-2.12 (m, 1H), 2.07-2.05 (m, 2H), 1.65-1.60 (m, 1H), 1.24 (s, 1H), 1.18-1.12 (m, 4H), 1.10-1.06 (m, 5H), 0.95 (s, 3H), 0.52 (s, 3H). LC-MS (M+H)⁺=877.3.

Example 38: ¹H NMR (400 MHz, DMSO) δ 8.44 (d, J=8.9 Hz, 1H), 8.04 (s, 1H), 7.95 (d, J=2.1 Hz, 1H), 7.78 (s, 1H), 7.16 (s, 1H), 6.69 (d, J=2.2 Hz, 1H), 5.91 (d, J=8 Hz, 1H), 5.33 (t, J=8.5 Hz, 1H), 4.72 (d, J=8 Hz, 1H), 4.66-4.54 (m, 2H), 4.50-4.49 (m, 1H), 4.35-4.27 (m, 2H), 3.878-3.86 (m, 2H), 3.81-3.80 (m, 2H), 3.70 (s, 1H), 3.58-3.53 (m, 2H), 3.22 (s, 3H), 3.16-3.07 (m, 1H), 2.94-2.90 (m, 1H), 2.67-2.65 (m, 4H), 2.44-2.43 (m, 2H), 2.34 (brs, 2H), 2.23 (s, 3H), 2.13-2.11 (m, 1H), 2.05 (s, 2H), 1.63-1.61 (m, 1H), 1.34-1.32 (m, 3H), 1.23-1.21 (m, 1H), 1.18-1.16 (m, 2H), 1.09-1.06 (m, 6H), 0.94 (s, 3H), 0.37 (s, 3H). LC-MS (M+H)⁺=877.3.

Atrop-39 & Example 39: (1R,2R,3S)—N-[(7S,13S,19P)-20-{5-[(9aR)-octahydropyrazino[2,1-c][1,4]oxazin-8-yl]-2-[(1S)-1-methoxyethyl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide & (1R,2R,3S)—N-[(7S,13S,19M)-20-{5-[(9aR)-octahydropyrazino[2,1-c][1,4]oxazin-8-yl]-2-[(1S)-1-methoxyethyl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

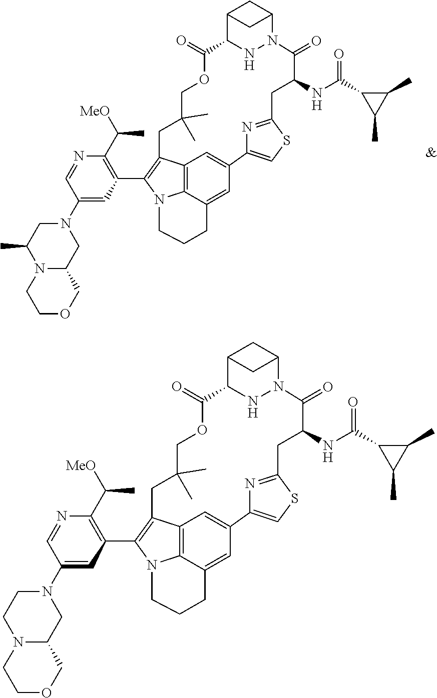

### Step 1: 3-(2-{5-[(9aR)-octahydropyrazino[2,1-c][1,4]oxazin-8-yl]-2-[(1S)-1-methoxyethyl]pyridin-3-yl}-6-bromo-1-azatricyclo[6.3.1.0^{4,12}]dodeca-2,4(12),5,7-tetraen-3-yl)-2,2-dimethylpropyl Acetate

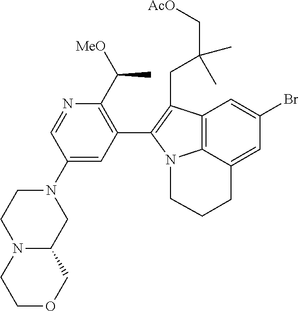 [SMILES: CO[C@@H](C)c1ncc(N2CCN3CCOC[C@H]3C2)cc1-c1c(CC(C)(C)COC(C)=O)c2cc(Br)cc3c2n1CCC3]

The title compound (180 mg, 28%) was prepared in a manner similar to that in example 9 step 2 from 3-(6-bromo-2-{2-[(1S)-1-methoxyethyl]-5-(4,4,5,5-tetramethyl-1,3,2-dioxaborolan-2-yl)pyridin-3-yl}-1-azatricyclo[6.3.1.0^{4,12}]dodeca-2,4(12),5,7-tetraen-3-yl)-2,2-dimethylpropyl acetate and (9aR)-octahydropyrazino[2,1-c][1,4]oxazine. LC-MS (M+H)⁺=639.3/641.4.

### Step 2: 3-(2-{5-[(9aR)-octahydropyrazino[2,1-c][1,4]oxazin-8-yl]-2-[(1S)-1-methoxyethyl]pyridin-3-yl}-6-(4,4,5,5-tetramethyl-1,3,2-dioxaborolan-2-yl)-1-azatricyclo[6.3.1.0^{4,12}]dodeca-2,4(12),5,7-tetraen-3-yl)-2,2-dimethylpropyl Acetate

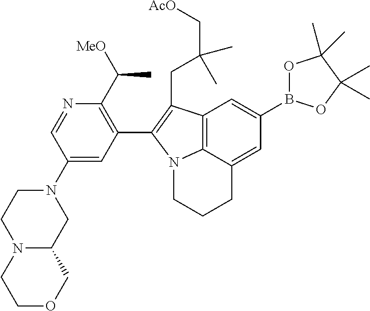 [SMILES: CO[C@@H](C)c1ncc(N2CCN3CCOC[C@H]3C2)cc1-c1c(CC(C)(C)COC(C)=O)c2cc(B3OC(C)(C)C(C)(C)O3)cc3c2n1CCC3]

The title compound (131 mg, 67%) was prepared in a manner similar to that in example 9 step 3 from 3-(2-{5-[(9aR)-octahydropyrazino[2,1-c][1,4]oxazin-8-yl]-2-[(1S)-1-methoxyethyl]pyridin-3-yl}-6-bromo-1-azatricyclo[6.3.1.0^{4,12}]dodeca-2,4(12),5,7-tetraen-3-yl)-2,2-dimethylpropyl acetate. LC-MS (M+H)⁺=687.5.

### Step 3: methyl (4S)-2-[(2S)-3-[4-(2-{5-[(9aR)-octahydropyrazino[2,1-c][1,4]oxazin-8-yl]-2-[(1S)-1-methoxyethyl]pyridin-3-yl}-3-[3-(acetyloxy)-2,2-dimethylpropyl]-1-azatricyclo[6.3.1.0^{4,12}]dodeca-2,4(12),5,7-tetraen-6-yl)-1,3-thiazol-2-yl]-2-{[(tert-butoxy)carbonyl]amino}propanoyl]-2,3-diazabicyclo[3.1.1]heptane-4-carboxylate

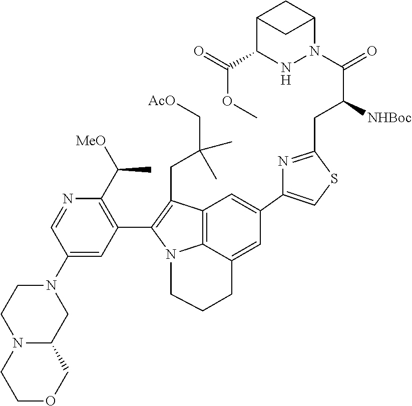 [SMILES: COC(=O)[C@H]1NN(C(=O)[C@H](Cc2nc(-c3cc4c5c(c3)c(CC(C)(C)COC(C)=O)c(-c3cc(N6CCN7CCOC[C@H]7C6)cnc3[C@H](C)OC)n5CCC4)cs2)NC(=O)OC(C)(C)C)C2CC1C2]

The title compound (171 mg, 93%) was prepared in a manner similar to that in example 9 step 4 from 3-(2-{5-[(9aR)-octahydropyrazino[2,1-c][1,4]oxazin-8-yl]-2-[(1S)-1-methoxyethyl]pyridin-3-yl}-6-(4,4,5,5-tetramethyl-1,3,2-dioxaborolan-2-yl)-1-azatricyclo[6.3.1.0^{4,12}]dodeca-2,4(12),5,7-tetraen-3-yl)-2,2-dimethylpropyl acetate and methyl (4S)-2-[(2S)-3-(4-bromo-1,3-thiazol-2-yl)-2-{[(tert-butoxy)carbonyl]amino}propanoyl]-2,3-diazabicyclo[3.1.1]heptane-4-carboxylate. LC-MS (M+H)⁺=969.7.

### Step 4: (4S)-2-[(2S)-3-[4-(2-{5-[(9aR)-octahydropyrazino[2,1-c][1,4]oxazin-8-yl]-2-[(1S)-1-methoxyethyl]pyridin-3-yl}-3-(3-hydroxy-2,2-dimethylpropyl)-1-azatricyclo[6.3.1.0^{4,12}]dodeca-2,4(12),5,7-tetraen-6-yl)-1,3-thiazol-2-yl]-2-{[(tert-butoxy)carbonyl]amino}propanoyl]-2,3-diazabicyclo[3.1.1]heptane-4-carboxylic Acid

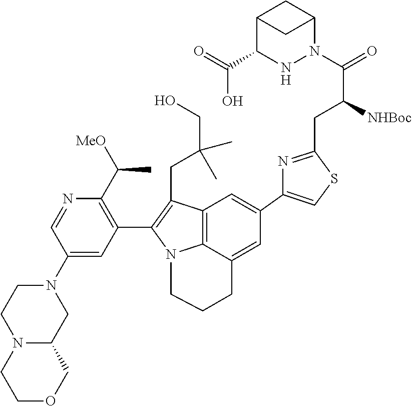 [SMILES: CO[C@@H](C)c1ncc(N2CCN3CCOC[C@H]3C2)cc1-c1c(CC(C)(C)CO)c2cc(-c3csc(C[C@H](NC(=O)OC(C)(C)C)C(=O)N4N[C@H](C(=O)O)C5CC4C5)n3)cc3c2n1CCC3]

The title compound (151 mg, 94%) was prepared in a manner similar to that in example 9 step 5 from methyl (4S)-2-[(2S)-3-[4-(2-{5-[(9aR)-octahydropyrazino[2,1-c][1,4]oxazin-8-yl]-2-[(1S)-1-methoxyethyl]pyridin-3-yl}-3-[3-(acetyloxy)-2,2-dimethylpropyl]-1-azatricyclo[6.3.1.0^{4,12}]dodeca-2,4(12),5,7-tetraen-6-yl)-1,3-thiazol-2-yl]-2-{[(tert-butoxy)carbonyl]amino}propanoyl]-2,3-diazabicyclo[3.1.1]heptane-4-carboxylate. LC-MS (M+H)⁺=913.7.

### Step 5: tert-butyl N-[(7S,13S)-20-{5-[(9aR)-octahydropyrazino[2,1-c][1,4]oxazin-8-yl]-2-[(1S)-1-methoxyethyl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate

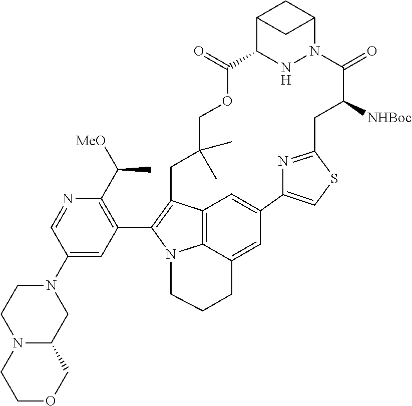 [SMILES: CO[C@@H](C)c1ncc(N2CCN3CCOC[C@H]3C2)cc1-c1c2c3cc(cc4c3n1CCC4)-c1csc(n1)C[C@H](NC(=O)OC(C)(C)C)C(=O)N1N[C@H](C(=O)OCC(C)(C)C2)C2CC1C2]

The title compound (45 mg, 30%) was prepared in a manner similar to that in example 9 step 6 from (4S)-2-[(2S)-3-[4-(2-{5-[(9aR)-octahydropyrazino[2,1-c][1,4]oxazin-8-yl]-2-[(1S)-1-methoxyethyl]pyridin-3-yl}-3-(3-hydroxy-2,2-dimethylpropyl)-1-azatricyclo[6.3.1.0^{4,12}]dodeca-2,4(12),5,7-tetraen-6-yl)-1,3-thiazol-2-yl]-2-{[(tert-butoxy)carbonyl]amino}propanoyl]-2,3-diazabicyclo[3.1.1]heptane-4-carboxylic acid. LC-MS (M+H)⁺=895.7.

### Step 6: (7S,13S)-20-{5-[(9aR)-octahydropyrazino[2,1-c][1,4]oxazin-8-yl]-2-[(1S)-1-methoxyethyl]pyridin-3-yl}-7-amino-17,17-dimethyl-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione

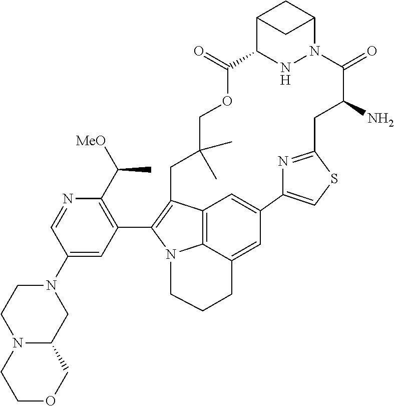 [SMILES: CO[C@@H](C)c1ncc(N2CCN3CCOC[C@H]3C2)cc1-c1c2c3cc(cc4c3n1CCC4)-c1csc(n1)C[C@H](N)C(=O)N1N[C@H](C(=O)OCC(C)(C)C2)C2CC1C2]

The title compound (40 mg, 99%) was prepared in a manner similar to that in example 9 step 7 from tert-butyl N-[(7S,13S)-20-{5-[(9aR)-octahydropyrazino[2,1-c][1,4]oxazin-8-yl]-2-[(1S)-1-methoxyethyl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate. LC-MS (M+H)⁺=795.5.

### Step 7: (1R,2R,3S)—N-[(7S,13S,19P)-20-{5-[(9aR)-octahydropyrazino[2,1-c][1,4]oxazin-8-yl]-2-[(1S)-1-methoxyethyl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide & (1R,2R,3S)—N-[(7S,13S,19M)-20-{5-[(9aR)-octahydropyrazino[2,1-c][1,4]oxazin-8-yl]-2-[(1S)-1-methoxyethyl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

Atrop-39 (P-isomer, 15.7 mg, 35%) and example 39 (M-isomer, 17.6 mg, 40%) were prepared in a manner similar to that in example 9 step 8 from (7S,13S)-20-{5-[(9aR)-octahydropyrazino[2,1-c][1,4]oxazin-8-yl]-2-[(1S)-1-methoxyethyl]pyridin-3-yl}-7-amino-17,17-dimethyl-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione and (1r,2R,3S)-2,3-dimethylcyclopropane-1-carboxylic acid.

Atrop-39: ¹H NMR (400 MHz, DMSO-d₆) δ 8.43-8.34 (d, J=9.0, 2H), 8.22 (s, 1H), 7.68 (s, 1H), 7.39 (s, 1H), 7.32 (d, J=2.4, 1H), 5.83 (d, J=11.2, 1H), 5.27 (t, J=8.2, 1H), 4.70 (d, J=11.0, 1H), 4.46 (dd, J=9.2, 4.6, 1H), 3.95-3.78 (m, 2H), 3.76-3.38 (m, 8H), 3.22-3.01 (m, 6H), 3.00-2.87 (m, 3H), 2.85-2.72 (m, 2H), 2.62 (d, J=6.6, 2H), 2.37-2.05 (m, 9H), 2.00-1.90 (m, 1H), 1.58 (t, J=9.4, 1H), 1.24-0.99 (m, 12H), 0.89 (s, 3H), 0.47 (s, 3H). LC-MS (M+H)⁺=891.7.

Example 39: ¹H NMR (400 MHz, DMSO-d₆) δ 8.36 (d, J=8.4, 2H), 8.20 (s, 1H), 7.67 (s, 1H), 7.39 (s, 1H), 7.13 (d, J=2.2, 1H), 5.88 (d, J=11.0, 1H), 5.34 (t, J=7.8, 1H), 4.62 (d, J=11.0, 1H), 4.44 (dd, J=9.6, 4.8, 1H), 4.27-4.08 (m, 2H), 3.80-3.39 (m, 8H), 3.24-3.05 (m, 6H), 3.02-2.92 (m, 1H), 2.90-2.69 (m, 4H), 2.66-2.55 (m, 2H), 2.32-2.02 (m, 9H), 1.51 (t, J=9.2, 1H), 1.30 (d, J=6.0, 3H), 1.23-0.93 (m, 10H), 0.85 (s, 3H), 0.35 (s, 3H). LC-MS (M+H)⁺=891.7.

Atrop-40 & Example 40: (1R,2R,3S)—N-[(7S,13S,19P)-20-{2-[(1S)-1-methoxyethyl]-5-[(5S)-5-methyl-4-oxa-7-azaspiro[2.5]octan-7-yl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide and (1R,2R,3S)—N-[(7S,13S,19M)-20-{2-[(1S)-1-methoxyethyl]-5-[(5S)-5-methyl-4-oxa-7-azaspiro[2.5]octan-7-yl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

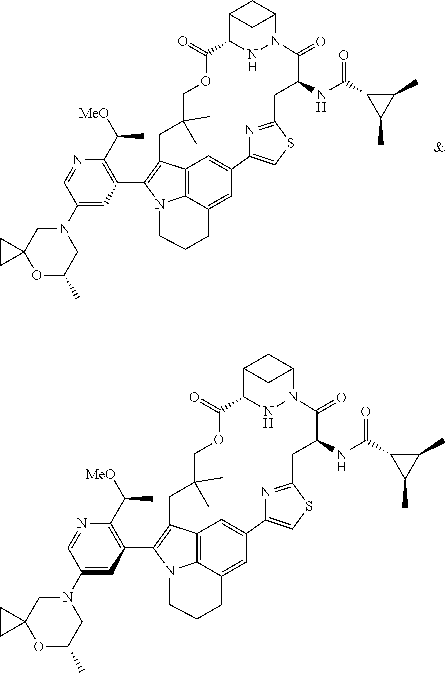

### Step 1: benzyl 5-methyl-4-oxa-7-azaspiro[2.5]octane-7-carboxylate

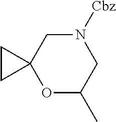 [SMILES: CC1CN(C(=O)OCc2ccccc2)CC2(CC2)O1]

To a solution of 5-methyl-4-oxa-7-azaspiro[2.5]octane (5.9 g, 46.5 mmol) in DCM (100 mL) were added TEA (9.4 g, 92.9 mmol) and CbzOSu (11.6 g, 46.5 mmol). The mixture was stirred at room temperature for 12 h and diluted with DCM (200 mL). The mixture was washed with brine (80 mL×2), dried over Na₂SO₄, filtered and concentrated under reduced pressure. The residue was purified by silica gel column chromatograph (PEEA=8/1) to give the title compound (7.6 g, 62%). LC-MS (M+H)⁺=262.3.

### Step 2: benzyl (5S)-5-methyl-4-oxa-7-azaspiro[2.5]octane-7-carboxylate and benzyl (5R)-5-methyl-4-oxa-7-azaspiro[2.5]octane-7-carboxylate

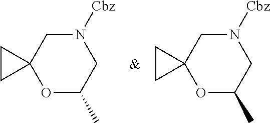 [SMILES: C[C@H]1CN(C(=O)OCc2ccccc2)CC2(CC2)O1 ; C[C@@H]1CN(C(=O)OCc2ccccc2)CC2(CC2)O1]

Benzyl 5-methyl-4-oxa-7-azaspiro[2.5]octane-7-carboxylate (7.6 g, 29.0 mmol) was separated by SFC (column: Lux Cellulose-4; Column Size: 3 cm×25 cm, 5 um; Mobile Phase A: CO₂; Mobile Phase B: MeCN (0.5% 2 mM NH₃-MeOH); Flow Rate: 100 mL/min; Wave Length: UV 220 nm; Temperature: 25° C.) to give benzyl (5S)-5-methyl-4-oxa-7-azaspiro[2.5]octane-7-carboxylate (2.8 g, 37/a, first peak) and benzyl (5R)-5-methyl-4-oxa-7-azaspiro[2.5]octane-7-carboxylate (3.0 g, 39%, second peak). LC-MS (M+H)⁺=262.3.

### Step 3: (5S)-5-methyl-4-oxa-7-azaspiro[2.5]octane

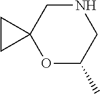

To a solution of benzyl (5S)-5-methyl-4-oxa-7-azaspiro[2.5]octane-7-carboxylate (2.8 g, 10.7 mmol) in MeOH (40 mL) was added Pd(OH)₂/C (300 mg) under nitrogen. The mixture was stirred for 10 h at room temperature under hydrogen. The filtrate was collected and concentrated to give the title compound (1.3 g, 96%). LC-MS (M+H)⁺=128.3.

### Step 4: 3-(6-bromo-2-{2-[(1S)-1-methoxyethyl]-5-[(5S)-5-methyl-4-oxa-7-azaspiro[2.5]octan-7-yl]pyridin-3-yl}-1-azatricyclo[6.3.1.0^{4,12}]dodeca-2,4(12),5,7-tetraen-3-yl)-2,2-dimethylpropyl Acetate

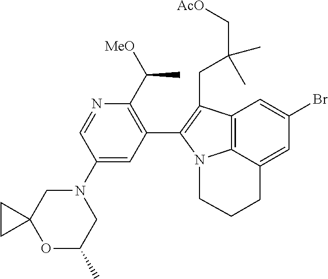 [SMILES: CO[C@@H](C)c1ncc(N2C[C@H](C)OC3(CC3)C2)cc1-c1c(CC(C)(C)COC(C)=O)c2cc(Br)cc3c2n1CCC3]

To a mixture of 3-(6-bromo-2-{2-[(1S)-1-methoxyethyl]-5-(4,4,5,5-tetramethyl-1,3,2-dioxaborolan-2-yl)pyridin-3-yl}-1-azatricyclo[6.3.1.0^{4,12}]dodeca-2,4(12),5,7-tetraen-3-yl)-2,2-dimethylpropyl acetate (1.1 g, 1.76 mmol) and (5S)-5-methyl-4-oxa-7-azaspiro[2.5]octane (447 mg, 3.52 mmol) in MeCN (15 mL) was added Et₃N (533 mg, 5.28 mmol) and Cu(OAc)₂ (640 mg, 3.52 mmol). The mixture was stirred for 4 h at room temperature under oxygen. The mixture was then diluted with EA (60 mL) and water (60 mL). Solid was filtered off and the filter cake was rinsed with EA (30 mL×2). The organic phase of the filtrate was separated and successively washed with water (50 mL) and brine (60 mL), dried over Na₂SO₄, filtered and concentrated under vacuum. The residue was purified by silica gel chromatograph (DCM:MeOH=15:1) to give the title compound (550 mg, 59%). LC-MS (M+H)⁺=624.5.

### Step 5: 3-(2-{2-[(1S)-1-methoxyethyl]-5-[(5S)-5-methyl-4-oxa-7-azaspiro[2.5]octan-7-yl]pyridin-3-yl}-6-(4,4,5,5-tetramethyl-1,3,2-dioxaborolan-2-yl)-1-azatricyclo[6.3.1.0^{4,12}]dodeca-2,4(12),5,7-tetraen-3-yl)-2,2-dimethylpropyl Acetate

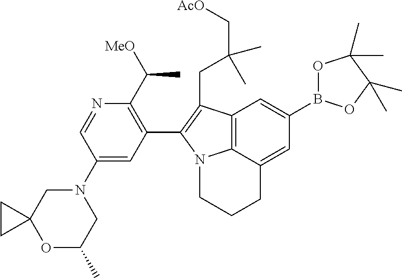 [SMILES: CO[C@@H](C)c1ncc(N2C[C@H](C)OC3(CC3)C2)cc1-c1c(CC(C)(C)COC(C)=O)c2cc(B3OC(C)(C)C(C)(C)O3)cc3c2n1CCC3]

To a mixture of 3-(6-bromo-2-{2-[(1S)-1-methoxyethyl]-5-[(5S)-5-methyl-4-oxa-7-azaspiro[2.5]octan-7-yl]pyridin-3-yl}-1-azatricyclo[6.3.1.0^{4,12}]dodeca-2,4(12),5,7-tetraen-3-yl)-2,2-dimethylpropyl acetate (550 mg, 0.88 mmol) and BPD (447 mg, 1.76 mmol) in dioxane (10 mL) was added Pd(dppf)Cl₂·DCM (72 mg, 0.088 mmol) and KOAc (259 mg, 2.64 mmol). The mixture was stirred for 4 h at 90° C. under nitrogen. The mixture was cooled to room temperature and diluted with EA (50 mL). The mixture was washed with brine (20 mL) dried over Na₂SO₄, filtered and concentrated under vacuum. The residue was purified by prep-TLC (DCM:MeOH=15:1) to give the title compound (280 mg, 47%). LC-MS (M+H)⁺=672.5.

### Step 6: methyl (4S)-2-[(2S)-3-(4-{3-[3-(acetyloxy)-2,2-dimethylpropyl]-2-{2-[(1S)-1-methoxyethyl]-5-[(5S)-5-methyl-4-oxa-7-azaspiro[2.5]octan-7-yl]pyridin-3-yl}-1-azatricyclo[6.3.1.0^{4,12}]dodeca-2,4(12),5,7-tetraen-6-yl}-1,3-thiazol-2-yl)-2-{[(tert-butoxy)carbonyl]amino}propanoyl]-2,3-diazabicyclo[3.1.1]heptane-4-carboxylate

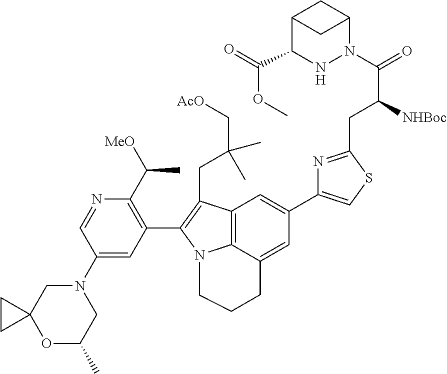 [SMILES: COC(=O)[C@H]1NN(C(=O)[C@H](Cc2nc(-c3cc4c5c(c3)c(CC(C)(C)COC(C)=O)c(-c3cc(N6C[C@H](C)OC7(CC7)C6)cnc3[C@H](C)OC)n5CCC4)cs2)NC(=O)OC(C)(C)C)C2CC1C2]

To a mixture of 3-(2-{2-[(1S)-1-methoxyethyl]-5-[(5S)-5-methyl-4-oxa-7-azaspiro[2.5]octan-7-yl]pyridin-3-yl}-6-(4,4,5,5-tetramethyl-1,3,2-dioxaborolan-2-yl)-1-azatricyclo[6.3.1.0^{4,12}]dodeca-2,4(12),5,7-tetraen-3-yl)-2,2-dimethylpropyl acetate (280 mg, 0.42 mmol) and methyl (4S)-2-[(2S)-3-(4-bromo-1,3-thiazol-2-yl)-2-{[(tert-butoxy)carbonyl]amino}propanoyl]-2,3-diazabicyclo[3.1.1]heptane-4-carboxylate (245 mg, 0.50 mmol) in dioxane (10 mL) and water (1 mL) was added K₃PO₄ (267 mg, 1.26 mmol) and Pd(dtbpf)Cl₂ (26 mg, 0.04 mmol). The mixture was stirred for 3 h at 70° C. under nitrogen and then cooled to room temperature. The mixture was diluted with EA (50 mL) and successively washed with water (20 mL) and brine (20 mL). The organic layer was dried over Na₂SO₄, filtered and concentrated under vacuum. The residue was purified by prep-TLC (DCM:MeOH=10:1) to give the title compound (350 mg, 88%). LC-MS (M+H)⁺=954.3.

### Step 7: (4S)-2-[(2S)-2-{[(tert-butoxy)carbonyl]amino}-3-{4-[3-(3-hydroxy-2,2-dimethylpropyl)-2-{2-[(1S)-1-methoxyethyl]-5-[(5S)-5-methyl-4-oxa-7-azaspiro[2.5]octan-7-yl]pyridin-3-yl}-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic Acid

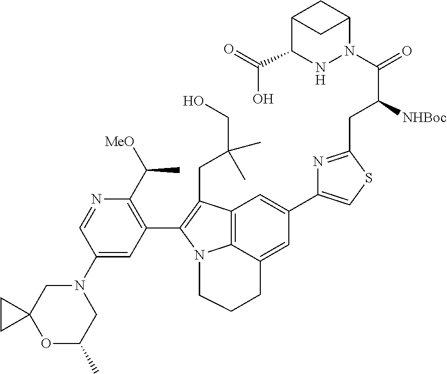 [SMILES: CO[C@@H](C)c1ncc(N2C[C@H](C)OC3(CC3)C2)cc1-c1c(CC(C)(C)CO)c2cc(-c3csc(C[C@H](NC(=O)OC(C)(C)C)C(=O)N4N[C@H](C(=O)O)C5CC4C5)n3)cc3c2n1CCC3]

To a mixture of methyl (4S)-2-[(2S)-3-(4-{3-[3-(acetyloxy)-2,2-dimethylpropyl]-2-{2-[(1S)-1-methoxyethyl]-5-[(5S)-5-methyl-4-oxa-7-azaspiro[2.5]octan-7-yl]pyridin-3-yl}-1-azatricyclo[6.3.1.0^{4,12}]dodeca-2,4(12),5,7-tetraen-6-yl}-1,3-thiazol-2-yl)-2-{[(tert-butoxy)carbonyl]amino}propanoyl]-2,3-diazabicyclo[3.1.1]heptane-4-carboxylate (350 mg, 0.37 mmol) in THF (5 mL) and water (5 mL) was added LiOH (44 mg, 1.83 mmol). The mixture was stirred for 6 h at room temperature. The mixture was diluted with water (40 mL) and washed with MTBE (10×2 mL). The organic phase was discarded, and the pH of the aqueous phase was adjusted to ˜5 with 1 N hydrochloric acid at 0° C. The mixture was extracted with EA (40 mL×3). The combined organic layer was dried over Na₂SO₄, filtered and concentrated under vacuum to give the title compound (250 mg, 76%). LC-MS (M+H)⁺=898.3.

### Step 8: tert-butyl N-[(7S,13S)-20-{2-[(1S)-1-methoxyethyl]-5-[(5S)-5-methyl-4-oxa-7-azaspiro[2.5]octan-7-yl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate

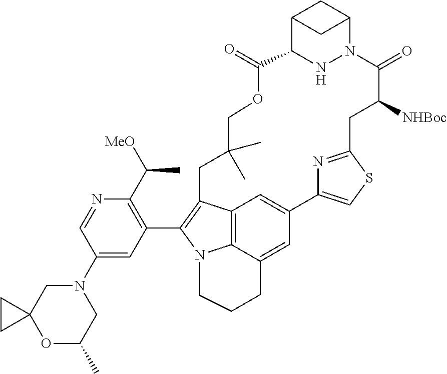 [SMILES: CO[C@@H](C)c1ncc(N2C[C@H](C)OC3(CC3)C2)cc1-c1c2c3cc(cc4c3n1CCC4)-c1csc(n1)C[C@H](NC(=O)OC(C)(C)C)C(=O)N1N[C@H](C(=O)OCC(C)(C)C2)C2CC1C2]

To a mixture of (4S)-2-[(2S)-2-{[(tert-butoxy)carbonyl]amino}-3-{4-[3-(3-hydroxy-2,2-dimethylpropyl)-2-{2-[(1S)-1-methoxyethyl]-5-[(5S)-5-methyl-4-oxa-7-azaspiro[2.5]octan-7-yl]pyridin-3-yl}-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic acid (250 mg, 0.28 mmol) in MeCN (100 mL) was added TCFH (234 mg, 0.84 mmol) and NMI (115 mg, 1.4 mmol). The mixture was stirred 2 h at room temperature and then concentrated under reduced pressure. The residue was dissolved in EA (40 mL), washed with 1 N hydrochloric acid (20 mL), water (20 mL) and brine (20 mL). The organic layer was dried over Na₂SO₄, filtered and concentrated under vacuum. The residue was purified by prep-TLC (DCM:MeOH=10:1) to give the title compound (120 mg, 49%). LC-MS (M+H)⁺=880.5.

### Step 9: (7S,13S)-7-amino-20-{2-[(1S)-1-methoxyethyl]-5-[(5S)-5-methyl-4-oxa-7-azaspiro[2.5]octan-7-yl]pyridin-3-yl}-17,17-dimethyl-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dine

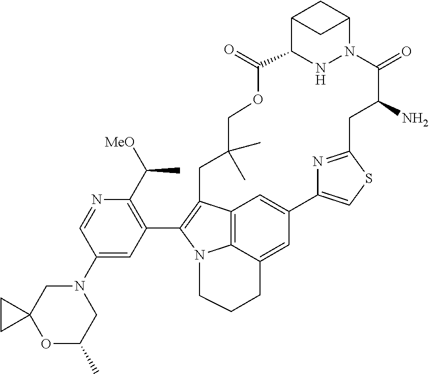 [SMILES: CO[C@@H](C)c1ncc(N2C[C@H](C)OC3(CC3)C2)cc1-c1c2c3cc(cc4c3n1CCC4)-c1csc(n1)C[C@H](N)C(=O)N1N[C@H](C(=O)OCC(C)(C)C2)C2CC1C2]

A mixture of tert-butyl N-[(7S,13S)-20-{2-[(1S)-1-methoxyethyl]-5-[(5S)-5-methyl-4-oxa-7-azaspiro[2.5]octan-7-yl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate (120 mg, 0.14 mmol) in HC (4 M in dioxane, 8 mL) was stirred for 1 h at room temperature and then concentrated under vacuum. The residue was diluted with DCM (20 mL), washed with saturated NaHCO₃ (10 mL), dried over Na₂SO₄, filtered and concentrated under vacuum to give the title compound (100 mg, 94%). LC-MS (M+H)⁺=780.5.

### Step 10: (1R,2R,3S)—N-[(7S,13S,19P)-20-{2-[(1S)-1-methoxyethyl]-5-[(5S)-5-methyl-4-oxa-7-azaspiro[2.5]octan-7-yl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide and (1R,2R,3S)—N-[(7S,13S,19M)-20-{2-[(1S)-1-methoxyethyl]-5-[(5S)-5-methyl-4-oxa-7-azaspiro[2.5]octan-7-yl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

To a mixture of (7S,13S)-7-amino-20-{2-[(1S)-1-methoxyethyl]-5-[(5S)-5-methyl-4-oxa-7-azaspiro[2.5]octan-7-yl]pyridin-3-yl}-17,17-dimethyl-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione (100 mg, 0.13 mmol) in DMF (4 mL) was added DIPEA (168 mg, 1.3 mmol), (1r,2R,3S)-2,3-dimethylcyclopropane-1-carboxylic acid (30 mg, 0.26 mmol) and HATU (99 mg, 0.26 mmol). The mixture was stirred for 2 h at room temperature and diluted with EA (40 mL), washed with brine (10 mL×2). The organic phase was dried over Na₂SO₄, filtered and concentrated under vacuum. The residue was purified by prep-TLC (DCM:MeOH=10:1) followed by prep-HPLC to give Atrop-40 (P-isomer, 28 mg, 25%) and example 40 (M-isomer, 31 mg, 28%).

Atrop-40: ¹H NMR (400 MHz, DMSO-d6) δ 8.39 (d, J=8.0 Hz, 1H), 8.35 (d, J=4.0 Hz, 1H), 8.23 (s, 1H), 7.68 (s, 1H), 7.39 (s, 1H), 7.35 (d, J=4.0 Hz, 1H), 5.84 (d, J=12.0 Hz, 1H), 5.30-5.24 (m, 1H), 4.70 (d, J=12.0 Hz, 1H), 4.49-4.43 (m, 1H), 3.96-3.90 (m, 1H), 3.88-3.74 (m, 3H), 3.59 (d, J=12.0 Hz, 1H), 3.49 (d, J=12.0 Hz, 1H), 3.45-3.39 (m, 1H), 3.21-3.16 (m, 1H), 3.15 (s, 2H), 3.12-3.08 (m, 1H), 3.05 (s, 3H), 2.99-2.91 (m, 3H), 2.65-2.60 (m, 1H), 2.38-2.27 (m, 3H), 2.22-2.15 (m, 1H), 2.14-2.07 (m, 1H), 2.02-1.91 (m, 1H), 1.60-1.54 (m, 1H), 1.26-1.16 (m, 2H), 1.14-1.12 (m, 1H), 1.09-1.01 (m, 13H), 0.89 (s, 3H), 0.79-0.75 (m, 1H), 0.64-0.57 (m, 2H), 0.55-0.51 (m, 1H), 0.47 (s, 3H). LC-MS (M+H)⁺=876.1.

Example 40: ¹H NMR (400 MHz, DMSO-d6) δ 8.44-8.33 (m, 2H), 8.20 (s, 1H), 7.68 (s, 1H), 7.39 (s, 1H), 7.13 (d, J=2.9 Hz, 1H), 5.89 (d, J=11.0 Hz, 1H), 5.33 (t, J=8.0 Hz, 1H), 4.63 (d, J=11.0 Hz, 1H), 4.45 (q, J=4.8 Hz, 1H), 4.33-4.09 (m, 2H), 3.88-3.70 (m, 2H), 3.63-3.39 (m, 3H), 3.24-3.04 (m, 7H), 3.03-2.79 (m, 3H), 2.66-2.54 (m, 1H), 2.46-2.03 (m, 7H), 1.51 (t, J=9.3 Hz, 1H), 1.30 (d, J=6.1 Hz, 3H), 1.24-1.10 (m, 3H), 1.09-0.97 (m, 9H), 0.86 (s, 3H), 0.78-0.50 (m, 4H), 0.36 (s, 3H). LC-MS (M+H)⁺=876.1.

Atrop-41 & Example 41: (1R,2R,3S)—N-[(7S,13S,19P)-20-{2-[(1S)-1-methoxyethyl]-5-[(5R)-5-methyl-4-oxa-7-azaspiro[2.5]octan-7-yl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide and (1R,2R,3S)—N-[(7S,13S,19M)-20-{2-[(1S)-1-methoxyethyl]-5-[(5R)-5-methyl-4-oxa-7-azaspiro[2.5]octan-7-yl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

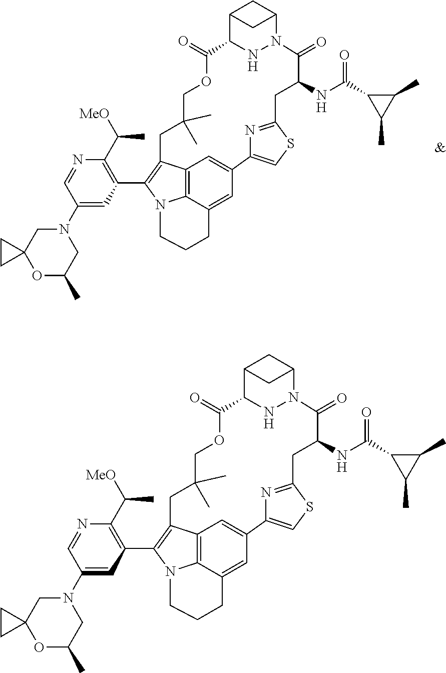

### Step 1: (5R)-5-methyl-4-oxa-7-azaspiro[2.5]octane

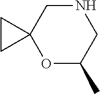

To a solution of benzyl (5R)-5-methyl-4-oxa-7-azaspiro[2.5]octane-7-carboxylate (3.0 g, 11.5 mmol) in MeOH (40 mL) was added Pd(OH)₂/C (300 mg) under nitrogen. The mixture was stirred for 10 h at room temperature under hydrogen. The reaction mixture was filtered through a short pad of celite. The filtrate was collected and concentrated to give the title compound (1.4 g, 96%). LC-MS (M+H)⁺=128.3.

### Step 2: 3-(6-bromo-2-{2-[(1S)-1-methoxyethyl]-5-[(5R)-5-methyl-4-oxa-7-azaspiro[2.5]octan-7-yl]pyridin-3-yl}-1-azatricyclo[6.3.1.0^{4,12}]dodeca-2,4(12),5,7-tetraen-3-yl)-2,2-dimethylpropyl Acetate

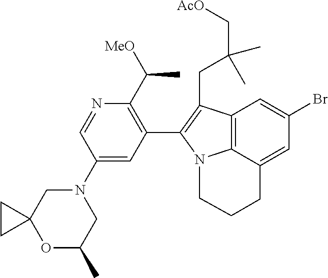 [SMILES: CO[C@@H](C)c1ncc(N2C[C@@H](C)OC3(CC3)C2)cc1-c1c(CC(C)(C)COC(C)=O)c2cc(Br)cc3c2n1CCC3]

To a mixture of 3-(6-bromo-2-{2-[(1S)-1-methoxyethyl]-5-(4,4,5,5-tetramethyl-1,3,2-dioxaborolan-2-yl)pyridin-3-yl}-1-azatricyclo[6.3.1.0^{4,12}]dodeca-2,4(12),5,7-tetraen-3-yl)-2,2-dimethylpropyl acetate (1.1 g, 1.76 mmol) and (5R)-5-methyl-4-oxa-7-azaspiro[2.5]octane (447 mg, 3.52 mmol) in MeCN (15 mL) was added Et₃N (533 mg, 5.28 mmol) and Cu(OAc)₂ (640 mg, 3.52 mmol). The mixture was stirred for 4 h at room temperature under oxygen. The mixture was then diluted with EA (60 mL) and water (60 mL). Solid was filtered off and the filter cake was rinsed with EA (30 mL×2). The organic phase of the filtrate was separated and successively washed with water (50 mL) and brine (60 mL), dried over Na₂SO₄, filtered and concentrated under vacuum. The residue was purified by silica gel chromatograph (DCM:MeOH=15:1) to give the title compound (560 mg, 60%). LC-MS (M+H)⁺=624.5.

### Step 3: 3-(2-{2-[(1S)-1-methoxyethyl]-5-[(5R)-5-methyl-4-oxa-7-azaspiro[2.5]octan-7-yl]pyridin-3-yl}-6-(4,4,5,5-tetramethyl-1,3,2-dioxaborolan-2-yl)-1-azatricyclo[6.3.1.0^{4,12}]dodeca-2,4(12),5,7-tetraen-3-yl)-2,2-dimethylpropyl Acetate

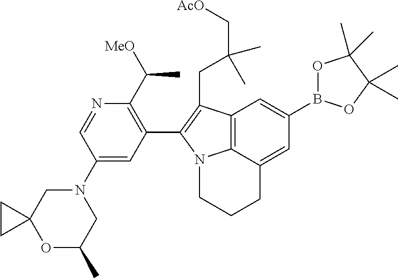 [SMILES: CO[C@@H](C)c1ncc(N2C[C@@H](C)OC3(CC3)C2)cc1-c1c(CC(C)(C)COC(C)=O)c2cc(B3OC(C)(C)C(C)(C)O3)cc3c2n1CCC3]

To a mixture of 3-(6-bromo-2-{2-[(1S)-1-methoxyethyl]-5-[(5R)-5-methyl-4-oxa-7-azaspiro[2.5]octan-7-yl]pyridin-3-yl}-1-azatricyclo[6.3.1.0^{4,12}]dodeca-2,4(12),5,7-tetraen-3-yl)-2,2-dimethylpropyl acetate (560 mg, 0.90 mmol) and BPD (447 mg, 1.76 mmol) in dioxane (10 mL) was added Pd(dppf)Cl₂·DCM (73 mg, 0.09 mmol) and KOAc (259 mg, 2.64 mmol). The mixture was stirred for 4 h at 90° C. under nitrogen. The mixture was cooled to room temperature and diluted with EA (50 mL). The mixture was washed with brine (20 mL) dried over Na₂SO₄, filtered and concentrated under vacuum. The residue was purified by prep-TLC (DCM:MeOH=15:1) to give the title compound (285 mg, 48%). LC-MS (M+H)⁺=672.5.

### Step 4: methyl (4S)-2-[(2S)-3-(4-{3-[3-(acetyloxy)-2,2-dimethylpropyl]-2-{2-[(1S)-1-methoxyethyl]-5-[(5R)-5-methyl-4-oxa-7-azaspiro[2.5]octan-7-yl]pyridin-3-yl}-1-azatricyclo[6.3.1.0^{4,12}]dodeca-2,4(12),5,7-tetraen-6-yl}-1,3-thiazol-2-yl)-2-{[(tert-butoxy)carbonyl]amino}propanoyl]-2,3-diazabicyclo[3.1.1]heptane-4-carboxylate

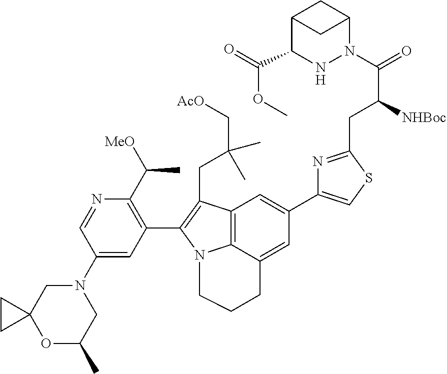 [SMILES: COC(=O)[C@H]1NN(C(=O)[C@H](Cc2nc(-c3cc4c5c(c3)c(CC(C)(C)COC(C)=O)c(-c3cc(N6C[C@@H](C)OC7(CC7)C6)cnc3[C@H](C)OC)n5CCC4)cs2)NC(=O)OC(C)(C)C)C2CC1C2]

To a mixture of 3-(2-{2-[(1S)-1-methoxyethyl]-5-[(5R)-5-methyl-4-oxa-7-azaspiro[2.5]octan-7-yl]pyridin-3-yl}-6-(4,4,5,5-tetramethyl-1,3,2-dioxaborolan-2-yl)-1-azatricyclo[6.3.1.0^{4,12}]dodeca-2,4(12),5,7-tetraen-3-yl)-2,2-dimethylpropyl acetate (285 mg, 0.42 mmol) and methyl (4S)-2-[(2S)-3-(4-bromo-1,3-thiazol-2-yl)-2-{[(tert-butoxy)carbonyl]amino}propanoyl]-2,3-diazabicyclo[3.1.1]heptane-4-carboxylate (245 mg, 0.50 mmol) in dioxane (10 mL) and water (1 mL) was added K₃PO₄ (267 mg, 1.26 mmol) and Pd(dtbpf)Cl₂ (26 mg, 0.04 mmol). The mixture was stirred for 3 h at 70° C. under nitrogen and then cooled to room temperature. The mixture was diluted with EA (50 mL) and successively washed with water (20 mL) and brine (20 mL). The organic layer was dried over Na₂SO₄, filtered and concentrated under vacuum. The residue was purified by prep-TLC (DCM:MeOH=10:1) to give the title compound (340 mg, 84%). LC-MS (M+H)⁺=954.3.

### Step 5: (4S)-2-[(2S)-2-{[(tert-butoxy)carbonyl]amino}-3-{4-[3-(3-hydroxy-2,2-dimethylpropyl)-2-{2-[(1S)-1-methoxyethyl]-5-[(5R)-5-methyl-4-oxa-7-azaspiro[2.5]octan-7-yl]pyridin-3-yl}-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic Acid

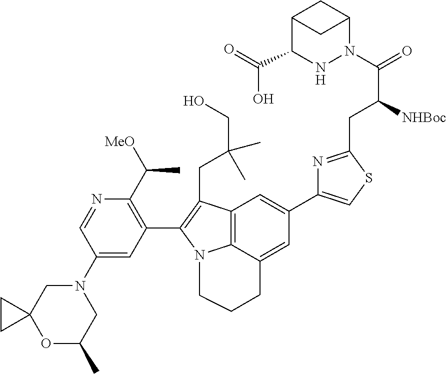 [SMILES: CO[C@@H](C)c1ncc(N2C[C@@H](C)OC3(CC3)C2)cc1-c1c(CC(C)(C)CO)c2cc(-c3csc(C[C@H](NC(=O)OC(C)(C)C)C(=O)N4N[C@H](C(=O)O)C5CC4C5)n3)cc3c2n1CCC3]

To a mixture of methyl (4S)-2-[(2S)-3-(4-{3-[3-(acetyloxy)-2,2-dimethylpropyl]-2-{2-[(1S)-1-methoxyethyl]-5-[(5R)-5-methyl-4-oxa-7-azaspiro[2.5]octan-7-yl]pyridin-3-yl}-1-azatricyclo[6.3.1.0^{4,12}]dodeca-2,4(12),5,7-tetraen-6-yl}-1,3-thiazol-2-yl)-2-{[(tert-butoxy)carbonyl]amino}propanoyl]-2,3-diazabicyclo[3.1.1]heptane-4-carboxylate (340 mg, 0.36 mmol) in THF (5 mL) and water (5 mL) was added LiOH (43 mg, 1.80 mmol). The mixture was stirred for 6 h at room temperature. The mixture was diluted with water (40 mL) and washed with MTBE (10×2 mL). The organic phase was discarded, and the pH of the aqueous phase was adjusted to ˜5 with 1 N hydrochloric acid at 0° C. The mixture was extracted with EA (40 mL×3). The combined organic layer was dried over Na₂SO₄, filtered and concentrated under vacuum to give the title compound (260 mg, 81%). LC-MS (M+H)⁺=898.3.

### Step 6: tert-butyl N-[(7S,13S)-20-{2-[(1S)-1-methoxyethyl]-5-[(5R)-5-methyl-4-oxa-7-azaspiro[2.5]octan-7-yl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate

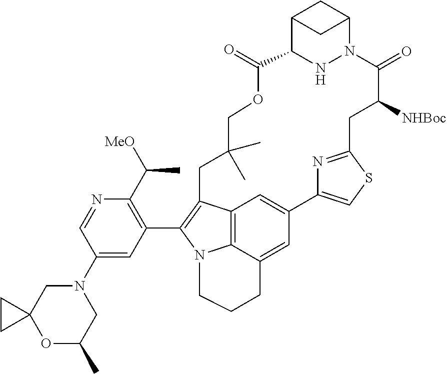 [SMILES: CO[C@@H](C)c1ncc(N2C[C@@H](C)OC3(CC3)C2)cc1-c1c2c3cc(cc4c3n1CCC4)-c1csc(n1)C[C@H](NC(=O)OC(C)(C)C)C(=O)N1N[C@H](C(=O)OCC(C)(C)C2)C2CC1C2]

To a mixture of (4S)-2-[(2S)-2-{[(tert-butoxy)carbonyl]amino}-3-{4-[3-(3-hydroxy-2,2-dimethylpropyl)-2-{2-[(1S)-1-methoxyethyl]-5-[(5R)-5-methyl-4-oxa-7-azaspiro[2.5]octan-7-yl]pyridin-3-yl}-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic acid (260 mg, 0.29 mmol) in MeCN (100 mL) was added TCFH (234 mg, 0.84 mmol) and NMI (115 mg, 1.4 mmol). The mixture was stirred 2 h at room temperature and then concentrated under reduced pressure. The residue was dissolved in EA (40 mL), washed with 1 N hydrochloric acid (20 mL), water (20 mL) and brine (20 mL). The organic layer was dried over Na₂SO₄, filtered and concentrated under vacuum. The residue was purified by prep-TLC (DCM:MeOH=10:1) to give the title compound (120 mg, 47%). LC-MS (M+H)⁺=880.5.

### Step 7: (7S,13S)-7-amino-20-{2-[(1S)-1-methoxyethyl]-5-[(5R)-5-methyl-4-oxa-7-azaspiro[2.5]octan-7-yl]pyridin-3-yl}-17,17-dimethyl-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione

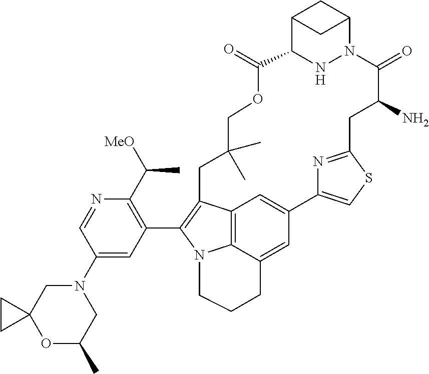 [SMILES: CO[C@@H](C)c1ncc(N2C[C@@H](C)OC3(CC3)C2)cc1-c1c2c3cc(cc4c3n1CCC4)-c1csc(n1)C[C@H](N)C(=O)N1N[C@H](C(=O)OCC(C)(C)C2)C2CC1C2]

A mixture of tert-butyl N-[(7S,13S)-20-{2-[(1S)-1-methoxyethyl]-5-[(5R)-5-methyl-4-oxa-7-azaspiro[2.5]octan-7-yl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate (120 mg, 0.14 mmol) in HC (4 M in dioxane, 8 mL) was stirred for 1 h at room temperature and then concentrated under vacuum. The residue was diluted with DCM (20 mL), washed with saturated NaHCO₃ (10 mL), dried over Na₂SO₄, filtered and concentrated under vacuum to give the title compound (100 mg, 94%). LC-MS (M+H)⁺=780.5.

### Step 8: (1R,2R,3S)—N-[(7S,13S,19P)-20-{2-[(1S)-1-methoxyethyl]-5-[(5R)-5-methyl-4-oxa-7-azaspiro[2.5]octan-7-yl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide and (1R,2R,3S)—N-[(7S,13S,19M)-20-{2-[(1S)-1-methoxyethyl]-5-[(5R)-5-methyl-4-oxa-7-azaspiro[2.5]octan-7-yl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

To a mixture of (7S,13S)-7-amino-20-{2-[(1S)-1-methoxyethyl]-5-[(5R)-5-methyl-4-oxa-7-azaspiro[2.5]octan-7-yl]pyridin-3-yl}-17,17-dimethyl-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione (100 mg, 0.13 mmol) in DMF (4 mL) was added DIPEA (168 mg, 1.3 mmol), (1r,2R,3S)-2,3-dimethylcyclopropane-1-carboxylic acid (30 mg, 0.26 mmol) and HATU (99 mg, 0.26 mmol). The mixture was stirred for 2 h at room temperature and diluted with EA (40 mL), washed with brine (10 mL×2). The organic phase was dried over Na₂SO₄, filtered and concentrated under vacuum. The residue was purified by prep-TLC (DCM:MeOH=10:1) followed by prep-HPLC to give Atrop-41 (P-isomer, 34 mg, 30%) and example 41 (M-isomer, 28 mg, 25%).

Atrop-41: ¹H NMR (400 MHz, DMSO-d6) δ 8.41-8.36 (m, 2H), 8.22 (s, 1H), 7.68 (s, 1H), 7.39 (s, 1H), 7.33 (d, J=4.0 Hz, 1H), 5.83 (d, J=12.0 Hz, 1H), 5.30-5.23 (m, 1H), 4.70 (d, J=12.0 Hz, 1H), 4.49-4.41 (m, 1H), 3.95-3.89 (m, 1H), 3.87-3.76 (m, 2H), 3.75-3.70 (m, 1H), 3.58 (d, J=12.0 Hz, 1H), 3.48 (d, J=12.0 Hz, 1H), 3.46-3.39 (m, 1H), 3.22-3.15 (m, 2H), 3.12-3.05 (m, 2H), 3.05 (s, 3H), 2.99-2.90 (m, 3H), 2.65-2.60 (m, 1H), 2.37-2.27 (m, 2H), 2.21-2.06 (m, 2H), 2.00-1.89 (m, 1H), 1.61-1.54 (m, 1H), 1.23-1.16 (m, 1H), 1.15-1.11 (m, 2H), 1.09-1.00 (m, 13H), 0.89 (s, 3H), 0.77-0.71 (m, 1H), 0.61-0.55 (m, 3H), 0.47 (s, 3H). LC-MS (M+H)⁺=876.1.

Example 41: ¹H NMR (400 MHz, DMSO-d6) δ 8.52-8.30 (m, 2H), 8.20 (d, J=1.3 Hz, 1H), 7.68 (s, 1H), 7.39 (s, 1H), 7.13 (d, J=2.9 Hz, 1H), 5.89 (d, J=11.0 Hz, 1H), 5.34 (t, J=8.2 Hz, 1H), 4.62 (d, J=11.0 Hz, 1H), 4.45 (q, J=4.9 Hz, 1H), 4.33-4.08 (m, 2H), 3.90-3.71 (m, 2H), 3.66-3.41 (m, 3H), 3.27-3.03 (m, 7H), 3.04-2.78 (m, 3H), 2.62-2.53 (m, 1H), 2.43-2.03 (m, 7H), 1.50 (t, J=9.4 Hz, 1H), 1.30 (d, J=6.1 Hz, 3H), 1.24-1.11 (m, 3H), 1.09-0.98 (m, 9H), 0.85 (s, 3H), 0.80-0.53 (m, 4H), 0.37 (s, 3H). LC-MS (M+H)⁺=876.1.

Atrop-42 & Example 42: (1R,2R,3S)—N-[(7S,13S,19P)-20-{2-[(1S)-1-methoxyethyl]-5-[(5S)-5-methyl-4-oxa-7-azaspiro[2.5]octan-7-yl]pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide and (1R,2R,3S)—N-[(7S,13S,19M)-20-{2-[(1S)-1-methoxyethyl]-5-[(5S)-5-methyl-4-oxa-7-azaspiro[2.5]octan-7-yl]pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

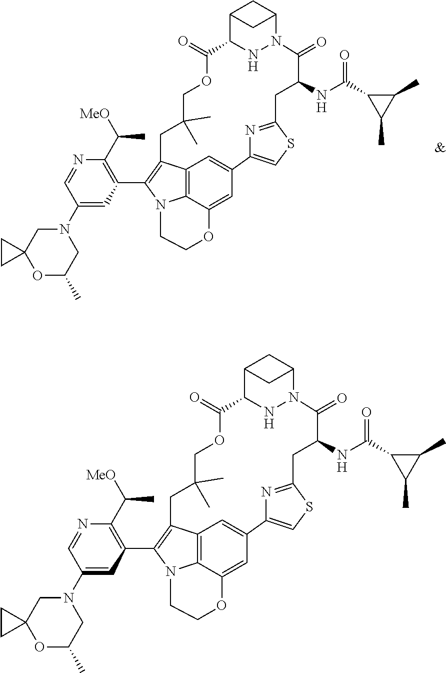

### Step 1: 3-(6-bromo-2-{2-[(1S)-1-methoxyethyl]-5-[(5S)-5-methyl-4-oxa-7-azaspiro[2.5]octan-7-yl]pyridin-3-yl}-9-oxa-1-azatricyclo[6.3.1.0^{4,12}]dodeca-2,4(12),5,7-tetraen-3-yl)-2,2-dimethylpropyl Acetate

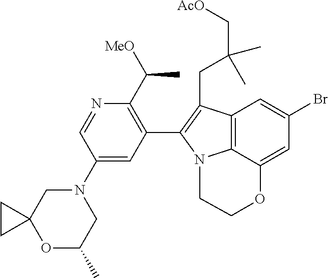 [SMILES: CO[C@@H](C)c1ncc(N2C[C@H](C)OC3(CC3)C2)cc1-c1c(CC(C)(C)COC(C)=O)c2cc(Br)cc3c2n1CCO3]

The title compound (300 mg, 48%) was prepared in a manner similar to that in example 41 step 2 from 3-(6-bromo-2-{2-[(1S)-1-methoxyethyl]-5-(4,4,5,5-tetramethyl-1,3,2-dioxaborolan-2-yl)pyridin-3-yl}-9-oxa-1-azatricyclo[6.3.1.0^{4,12}]dodeca-2,4(12),5,7-tetraen-3-yl)-2,2-dimethylpropyl acetate and (5S)-5-methyl-4-oxa-7-azaspiro[2.5]octane. LC-MS (M+H)⁺=626.3/628.3.

### Step 2: 3-(2-{2-[(1S)-1-methoxyethyl]-5-[(5S)-5-methyl-4-oxa-7-azaspiro[2.5]octan-7-yl]pyridin-3-yl}-6-(4,4,5,5-tetramethyl-1,3,2-dioxaborolan-2-yl)-9-oxa-1-azatricyclo[6.3.1.0^{4,12}]dodeca-2,4(12),5,7-tetraen-3-yl)-2,2-dimethylpropyl Acetate

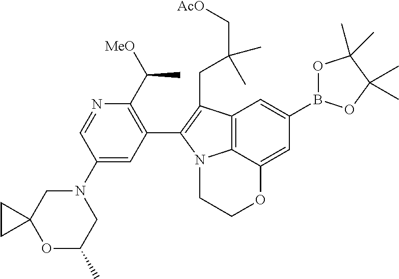 [SMILES: CO[C@@H](C)c1ncc(N2C[C@H](C)OC3(CC3)C2)cc1-c1c(CC(C)(C)COC(C)=O)c2cc(B3OC(C)(C)C(C)(C)O3)cc3c2n1CCO3]

The title compound (240 mg, 74%) was prepared in a manner similar to that in example 41 step 3 from 3-(6-bromo-2-{2-[(1S)-1-methoxyethyl]-5-[(5S)-5-methyl-4-oxa-7-azaspiro[2.5]octan-7-yl]pyridin-3-yl}-9-oxa-1-azatricyclo[6.3.1.0^{4,12}]dodeca-2,4(12),5,7-tetraen-3-yl)-2,2-dimethylpropyl acetate. LC-MS (M+H)⁺=674.5.

### Step 3: methyl (4S)-2-[(2S)-3-(4-{3-[3-(acetyloxy)-2,2-dimethylpropyl]-2-{2-[(1S)-1-methoxyethyl]-5-[(5S)-5-methyl-4-oxa-7-azaspiro[2.5]octan-7-yl]pyridin-3-yl}-9-oxa-1-azatricyclo[6.3.1.0^{4,12}]dodeca-2,4(12),5,7-tetraen-6-yl}-1,3-thiazol-2-yl)-2-{[(tert-butoxy)carbonyl]amino}propanoyl]-2,3-diazabicyclo[3.1.1]heptane-4-carboxylate

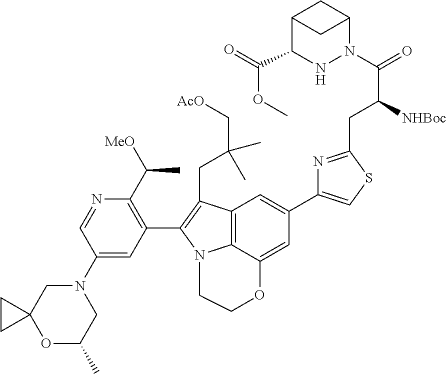 [SMILES: COC(=O)[C@H]1NN(C(=O)[C@H](Cc2nc(-c3cc4c5c(c3)c(CC(C)(C)COC(C)=O)c(-c3cc(N6C[C@H](C)OC7(CC7)C6)cnc3[C@H](C)OC)n5CCO4)cs2)NC(=O)OC(C)(C)C)C2CC1C2]

The title compound (260 mg, 76%) was prepared in a manner similar to that in example 41 step 4 from 3-(2-{2-[(1S)-1-methoxyethyl]-5-[(5S)-5-methyl-4-oxa-7-azaspiro[2.5]octan-7-yl]pyridin-3-yl}-6-(4,4,5,5-tetramethyl-1,3,2-dioxaborolan-2-yl)-9-oxa-1-azatricyclo[6.3.1.0^{4,12}]dodeca-2,4(12),5,7-tetraen-3-yl)-2,2-dimethylpropyl acetate and methyl (4S)-2-[(2S)-3-(4-bromo-1,3-thiazol-2-yl)-2-{[(tert-butoxy)carbonyl]amino}propanoyl]-2,3-diazabicyclo[3.1.1]heptane-4-carboxylate. LC-MS (M+H)⁺=956.5.

### Step 4: (4S)-2-[(2S)-2-{[(tert-butoxy)carbonyl]amino}-3-{4-[3-(3-hydroxy-2,2-dimethylpropyl)-2-{2-[(1S)-1-methoxyethyl]-5-[(5S)-5-methyl-4-oxa-7-azaspiro[2.5]octan-7-yl]pyridin-3-yl}-9-oxa-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic Acid

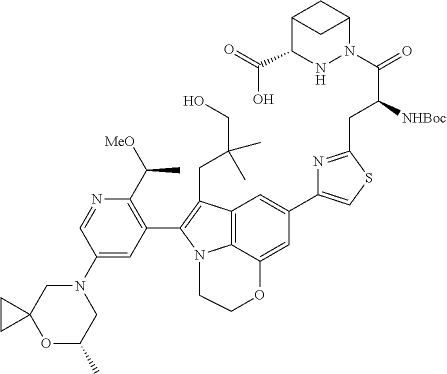 [SMILES: CO[C@@H](C)c1ncc(N2C[C@H](C)OC3(CC3)C2)cc1-c1c(CC(C)(C)CO)c2cc(-c3csc(C[C@H](NC(=O)OC(C)(C)C)C(=O)N4N[C@H](C(=O)O)C5CC4C5)n3)cc3c2n1CCO3]

The title compound (140 mg, 57%) was prepared in a manner similar to that in example 41 step 5 from methyl (4S)-2-[(2S)-3-(4-{3-[3-(acetyloxy)-2,2-dimethylpropyl]-2-{2-[(1S)-1-methoxyethyl]-5-[(5S)-5-methyl-4-oxa-7-azaspiro[2.5]octan-7-yl]pyridin-3-yl}-9-oxa-1-azatricyclo[6.3.1.0^{4,12}]dodeca-2,4(12),5,7-tetraen-6-yl}-1,3-thiazol-2-yl)-2-{[(tert-butoxy)carbonyl]amino}propanoyl]-2,3-diazabicyclo[3.1.1]heptane-4-carboxylate. LC-MS (M+H)⁺=900.5.

### Step 5: tert-butyl N-[(7S,13S)-20-{2-[(1S)-1-methoxyethyl]-5-[(5S)-5-methyl-4-oxa-7-azaspiro[2.5]octan-7-yl]pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate

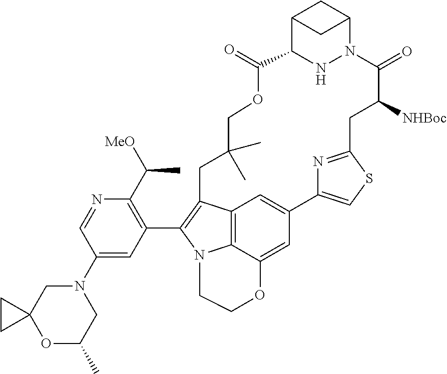 [SMILES: CO[C@@H](C)c1ncc(N2C[C@H](C)OC3(CC3)C2)cc1-c1c2c3cc(cc4c3n1CCO4)-c1csc(n1)C[C@H](NC(=O)OC(C)(C)C)C(=O)N1N[C@H](C(=O)OCC(C)(C)C2)C2CC1C2]

The title compound (80 mg, 58%) was prepared in a manner similar to that in example 41 step 6 from (4S)-2-[(2S)-2-{[(tert-butoxy)carbonyl]amino}-3-{4-[3-(3-hydroxy-2,2-dimethylpropyl)-2-{2-[(1S)-1-methoxyethyl]-5-[(5S)-5-methyl-4-oxa-7-azaspiro[2.5]octan-7-yl]pyridin-3-yl}-9-oxa-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic acid. LC-MS (M+H)⁺=882.5.

### Step 6: (7S,13S)-7-amino-20-{2-[(1S)-1-methoxyethyl]-5-[(5S)-5-methyl-4-oxa-7-azaspiro[2.5]octan-7-yl]pyridin-3-yl}-17,17-dimethyl-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione

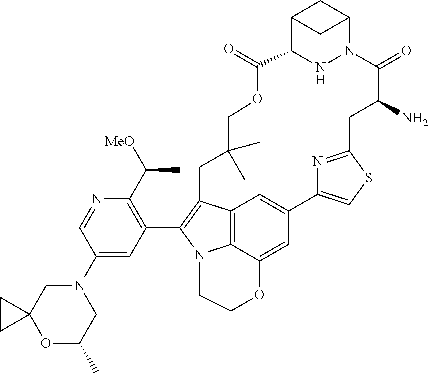 [SMILES: CO[C@@H](C)c1ncc(N2C[C@H](C)OC3(CC3)C2)cc1-c1c2c3cc(cc4c3n1CCO4)-c1csc(n1)C[C@H](N)C(=O)N1N[C@H](C(=O)OCC(C)(C)C2)C2CC1C2]

The title compound (65 mg, 92%) was prepared in a manner similar to that in example 41 step 7 from tert-butyl N-[(7S,13S)-20-{2-[(1S)-1-methoxyethyl]-5-[(5S)-5-methyl-4-oxa-7-azaspiro[2.5]octan-7-yl]pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate. LC-MS (M+H)⁺=782.5.

### Step 7: (1R,2R,3S)—N-[(7S,13S,19P)-20-{2-[(1S)-1-methoxyethyl]-5-[(5S)-5-methyl-4-oxa-7-azaspiro[2.5]octan-7-yl]pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide and (1R,2R,3S)—N-[(7S,13S,19M)-20-{2-[(1S)-1-methoxyethyl]-5-[(5S)-5-methyl-4-oxa-7-azaspiro[2.5]octan-7-yl]pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

Atrop-42 (P-isomer, 12 mg, 16%) and example 42 (M-isomer, 23 mg, 32%) were prepared in a manner similar to that in example 9 step 8 from (7S,13S)-7-amino-20-{2-[(1S)-1-methoxyethyl]-5-[(5S)-5-methyl-4-oxa-7-azaspiro[2.5]octan-7-yl]pyridin-3-yl}-17,17-dimethyl-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione and (1r,2R,3S)-2,3-dimethylcyclopropane-1-carboxylic acid.

Atrop-42: ¹H NMR (400 MHz, DMSO-d6) δ 8.40-8.33 (m, 2H), 8.03 (s, 1H), 7.73 (s, 1H), 7.37 (d, J=4.0 Hz, 1H), 7.12 (s, 1H), 5.82 (d, J=12.0 Hz, 1H), 5.29-5.22 (m, 1H), 4.70 (d, J=12.0 Hz, 1H), 4.51-4.43 (m, 2H), 4.41-4.32 (m, 1H), 4.08-3.97 (m, 2H), 3.82-3.74 (m, 2H), 3.66-3.60 (m, 1H), 3.58 (d, J=12.0 Hz, 1H), 3.47 (d, J=12.0 Hz, 1H), 3.22-3.18 (m, 1H), 3.17-3.14 (m, 2H), 3.12-3.05 (m, 4H), 2.99-2.93 (m, 1H), 2.66-2.60 (m, 1H), 2.44-2.26 (m, 4H), 2.13-2.05 (m, 1H), 1.61-1.54 (m, 1H), 1.22-1.16 (m, 1H), 1.13-1.02 (m, 14H), 0.90 (s, 3H), 0.79-0.72 (m, 1H), 0.63-0.57 (m, 2H), 0.55-0.48 (m, 1H), 0.45 (s, 3H). LC-MS (M+H)⁺=878.1.

Example 42: ¹H NMR (400 MHz, DMSO-d6) δ 8.53-8.32 (m, 2H), 8.00 (s, 1H), 7.73 (s, 1H), 7.29-6.97 (m, 2H), 5.86 (d, J=11.0 Hz, 1H), 5.29 (t, J=8.1 Hz, 1H), 4.66 (d, J=11.0 Hz, 1H), 4.61-4.40 (m, 3H), 4.34-4.20 (m, 2H), 3.85-3.74 (m, 2H), 3.70-3.46 (m, 3H), 3.24-3.02 (m, 7H), 2.88 (d, J=14.3 Hz, 1H), 2.61 (q, J=5.6 Hz, 1H), 2.42-2.04 (m, 5H), 1.55 (t, J=9.4 Hz, 1H), 1.37-1.27 (m, 3H), 1.22-1.10 (m, 3H), 1.10-0.95 (m, 9H), 0.89 (s, 3H), 0.78-0.44 (m, 4H), 0.34 (s, 3H). LC-MS (M+H)⁺=878.1.

Atrop-43 & Example 43: (1R,2R,3S)—N-[(7S,13S,19P)-20-{2-[(1S)-1-methoxyethyl]-5-[(5R)-5-methyl-4-oxa-7-azaspiro[2.5]octan-7-yl]pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide and (1R,2R,3S)—N-[(7S,13S,19M)-20-{2-[(1S)-1-methoxyethyl]-5-[(5R)-5-methyl-4-oxa-7-azaspiro[2.5]octan-7-yl]pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

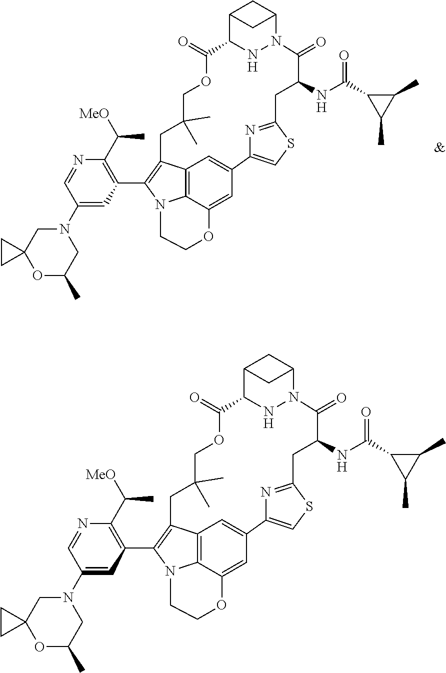

### Step 1: 3-(6-bromo-2-{2-[(1S)-1-methoxyethyl]-5-[(5R)-5-methyl-4-oxa-7-azaspiro[2.5]octan-7-yl]pyridin-3-yl}-9-oxa-1-azatricyclo[6.3.1.0^{4,12}]dodeca-2,4(12),5,7-tetraen-3-yl)-2,2-dimethylpropyl Acetate

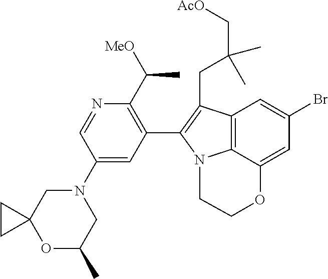 [SMILES: CO[C@@H](C)c1ncc(N2C[C@@H](C)OC3(CC3)C2)cc1-c1c(CC(C)(C)COC(C)=O)c2cc(Br)cc3c2n1CCO3]

The title compound (320 mg, 52%) was prepared in a manner similar to that in example 41 step 2 from 3-(6-bromo-2-{2-[(1S)-1-methoxyethyl]-5-(4,4,5,5-tetramethyl-1,3,2-dioxaborolan-2-yl)pyridin-3-yl}-9-oxa-1-azatricyclo[6.3.1.0^{4,12}]dodeca-2,4(12),5,7-tetraen-3-yl)-2,2-dimethylpropyl acetate and (5R)-5-methyl-4-oxa-7-azaspiro[2.5]octane. LC-MS (M+H)⁺=626.3/628.3.

### Step 2: 3-(2-{2-[(1S)-1-methoxyethyl]-5-[(5R)-5-methyl-4-oxa-7-azaspiro[2.5]octan-7-yl]pyridin-3-yl}-6-(4,4,5,5-tetramethyl-1,3,2-dioxaborolan-2-yl)-9-oxa-1-azatricyclo[6.3.1.0^{4,12}]dodeca-2,4(12),5,7-tetraen-3-yl)-2,2-dimethylpropyl Acetate

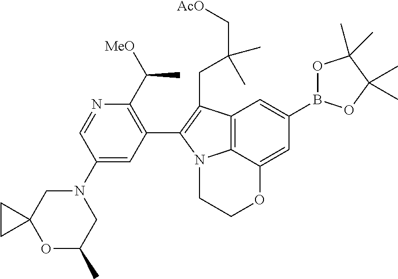 [SMILES: CO[C@@H](C)c1ncc(N2C[C@@H](C)OC3(CC3)C2)cc1-c1c(CC(C)(C)COC(C)=O)c2cc(B3OC(C)(C)C(C)(C)O3)cc3c2n1CCO3]

The title compound (260 mg, 76%) was prepared in a manner similar to that in example 41 step 3 from 3-(6-bromo-2-{2-[(1S)-1-methoxyethyl]-5-[(5R)-5-methyl-4-oxa-7-azaspiro[2.5]octan-7-yl]pyridin-3-yl}-9-oxa-1-azatricyclo[6.3.1.0^{4,12}]dodeca-2,4(12),5,7-tetraen-3-yl)-2,2-dimethylpropyl acetate. LC-MS (M+H)⁺=674.5.

### Step 3: methyl (4S)-2-[(2S)-3-(4-{3-[3-(acetyloxy)-2,2-dimethylpropyl]-2-{2-[(1S)-1-methoxyethyl]-5-[(5R)-5-methyl-4-oxa-7-azaspiro[2.5]octan-7-yl]pyridin-3-yl}-9-oxa-1-azatricyclo[6.3.1.0^{4,12}]dodeca-2,4(12),5,7-tetraen-6-yl}-1,3-thiazol-2-yl)-2-{[(tert-butoxy)carbonyl]amino}propanoyl]-2,3-diazabicyclo[3.1.1]heptane-4-carboxylate

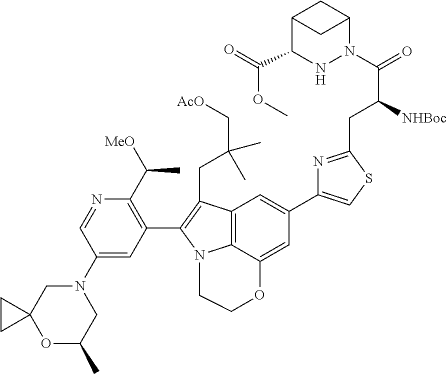 [SMILES: COC(=O)[C@H]1NN(C(=O)[C@H](Cc2nc(-c3cc4c5c(c3)c(CC(C)(C)COC(C)=O)c(-c3cc(N6C[C@@H](C)OC7(CC7)C6)cnc3[C@H](C)OC)n5CCO4)cs2)NC(=O)OC(C)(C)C)C2CC1C2]

The title compound (290 mg, 79%) was prepared in a manner similar to that in example 41 step 4 from 3-(2-{2-[(1S)-1-methoxyethyl]-5-[(5R)-5-methyl-4-oxa-7-azaspiro[2.5]octan-7-yl]pyridin-3-yl}-6-(4,4,5,5-tetramethyl-1,3,2-dioxaborolan-2-yl)-9-oxa-1-azatricyclo[6.3.1.0^{4,12}]dodeca-2,4(12),5,7-tetraen-3-yl)-2,2-dimethylpropyl acetate and methyl (4S)-2-[(2S)-3-(4-bromo-1,3-thiazol-2-yl)-2-{[(tert-butoxy)carbonyl]amino}propanoyl]-2,3-diazabicyclo[3.1.1]heptane-4-carboxylate. LC-MS (M+H)⁺=956.5.

### Step 4: (4S)-2-[(2S)-2-{[(tert-butoxy)carbonyl]amino}-3-{4-[3-(3-hydroxy-2,2-dimethylpropyl)-2-{2-[(1S)-1-methoxyethyl]-5-[(5R)-5-methyl-4-oxa-7-azaspiro[2.5]octan-7-yl]pyridin-3-yl}-9-oxa-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic Acid

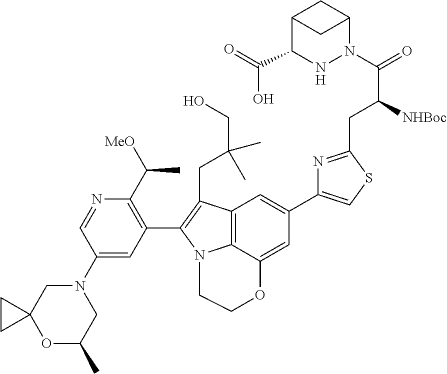 [SMILES: CO[C@@H](C)c1ncc(N2C[C@@H](C)OC3(CC3)C2)cc1-c1c(CC(C)(C)CO)c2cc(-c3csc(C[C@H](NC(=O)OC(C)(C)C)C(=O)N4N[C@H](C(=O)O)C5CC4C5)n3)cc3c2n1CCO3]

The title compound (170 mg, 62%) was prepared in a manner similar to that in example 41 step 5 from methyl (4S)-2-[(2S)-3-(4-{3-[3-(acetyloxy)-2,2-dimethylpropyl]-2-{2-[(1S)-1-methoxyethyl]-5-[(5R)-5-methyl-4-oxa-7-azaspiro[2.5]octan-7-yl]pyridin-3-yl}-9-oxa-1-azatricyclo[6.3.1.0^{4,12}]dodeca-2,4(12),5,7-tetraen-6-yl}-1,3-thiazol-2-yl)-2-{[(tert-butoxy)carbonyl]amino}propanoyl]-2,3-diazabicyclo[3.1.1]heptane-4-carboxylate. LC-MS (M+H)⁺=900.5.

### Step 5: tert-butyl N-[(7S,13S)-20-{2-[(1S)-1-methoxyethyl]-5-[(5R)-5-methyl-4-oxa-7-azaspiro[2.5]octan-7-yl]pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate

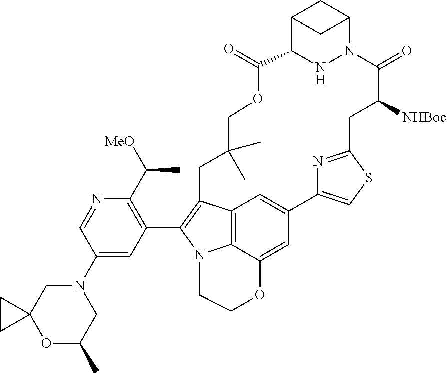 [SMILES: CO[C@@H](C)c1ncc(N2C[C@@H](C)OC3(CC3)C2)cc1-c1c2c3cc(cc4c3n1CCO4)-c1csc(n1)C[C@H](NC(=O)OC(C)(C)C)C(=O)N1N[C@H](C(=O)OCC(C)(C)C2)C2CC1C2]

The title compound (90 mg, 54%) was prepared in a manner similar to that in example 41 step 6 from (4S)-2-[(2S)-2-{[(tert-butoxy)carbonyl]amino}-3-{4-[3-(3-hydroxy-2,2-dimethylpropyl)-2-{2-[(1S)-1-methoxyethyl]-5-[(5R)-5-methyl-4-oxa-7-azaspiro[2.5]octan-7-yl]pyridin-3-yl}-9-oxa-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic acid. LC-MS (M+H)⁺=882.5.

### Step 6: (7S,13S)-7-amino-20-{2-[(1S)-1-methoxyethyl]-5-[(5R)-5-methyl-4-oxa-7-azaspiro[2.5]octan-7-yl]pyridin-3-yl}-17,17-dimethyl-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione

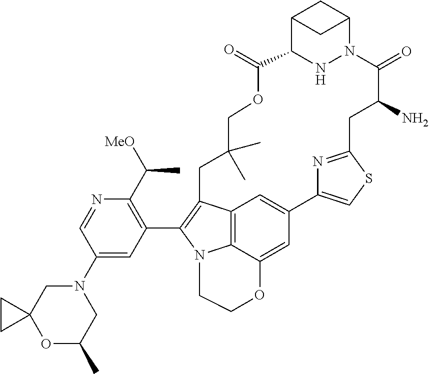 [SMILES: CO[C@@H](C)c1ncc(N2C[C@@H](C)OC3(CC3)C2)cc1-c1c2c3cc(cc4c3n1CCO4)-c1csc(n1)C[C@H](N)C(=O)N1N[C@H](C(=O)OCC(C)(C)C2)C2CC1C2]

The title compound (70 mg, 88%) was prepared in a manner similar to that in example 41 step 7 from tert-butyl N-[(7S,13S)-20-{2-[(1S)-1-methoxyethyl]-5-[(5R)-5-methyl-4-oxa-7-azaspiro[2.5]octan-7-yl]pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate. LC-MS (M+H)⁺=782.5.

### Step 7: (1R,2R,3S)—N-[(7S,13S,19P)-20-{2-[(1S)-1-methoxyethyl]-5-[(5R)-5-methyl-4-oxa-7-azaspiro[2.5]octan-7-yl]pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide and (1R,2R,3S)—N-[(7S,13S,19M)-20-{2-[(1S)-1-methoxyethyl]-5-[(5R)-5-methyl-4-oxa-7-azaspiro[2.5]octan-7-yl]pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

Atrop-43 (P-isomer, 15 mg, 19%) and example 43 (M-isomer, 27 mg, 35%) were prepared in a manner similar to that in example 9 step 8 from (7S,13S)-7-amino-20-{2-[(1S)-1-methoxyethyl]-5-[(5R)-5-methyl-4-oxa-7-azaspiro[2.5]octan-7-yl]pyridin-3-yl}-17,17-dimethyl-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione and (1r,2R,3S)-2,3-dimethylcyclopropane-1-carboxylic acid.

Atrop-43: ¹H NMR (400 MHz, DMSO-d6) δ 8.40-8.35 (m, 2H), 8.03 (s, 1H), 7.73 (s, 1H), 7.36 (d, J=4.0 Hz, 1H), 7.12 (s, 1H), 5.82 (d, J=12.0 Hz, 1H), 5.29-5.23 (m, 1H), 4.72-4.67 (m, 1H), 4.51-4.43 (m, 2H), 4.41-4.33 (m, 1H), 4.05-3.97 (m, 2H), 3.81-3.70 (m, 2H), 3.66-3.54 (m, 2H), 3.51-3.45 (m, 1H), 3.23-3.15 (m, 3H), 3.12-3.07 (m, 4H), 2.99-2.93 (m, 1H), 2.66-2.60 (m, 1H), 2.44-2.25 (m, 4H), 2.13-2.06 (m, 1H), 1.61-1.54 (m, 1H), 1.23-1.18 (m, 1H), 1.13-1.01 (m, 14H), 0.90 (s, 3H), 0.77-0.72 (m, 1H), 0.62-0.55 (m, 3H), 0.46 (s, 3H). LC-MS (M+H)⁺=878.1.

Example 43: 1H NMR (400 MHz, DMSO-d6) δ 8.51-8.23 (m, 2H), 8.00 (d, J=2.4 Hz, 1H), 7.72 (d, J=2.5 Hz, 1H), 7.27-7.02 (m, 2H), 5.86 (d, J=11.0 Hz, 1H), 5.30 (t, J=8.2 Hz, 1H), 4.76-4.38 (m, 4H), 4.33-4.12 (m, 2H), 3.89-3.40 (m, 5H), 3.22-3.02 (m, 7H), 2.86 (d, J=14.3 Hz, 1H), 2.67-2.54 (m, 1H), 2.51-2.01 (m, 5H), 1.53 (t, J=9.3 Hz, 1H), 1.33-1.28 (m, 3H), 1.24-0.97 (m, 12H), 0.87 (s, 3H), 0.79-0.47 (m, 4H), 0.34 (s, 3H). LC-MS (M+H)⁺=878.1.

### Example 44: (1R,2R,3S)—N-[(7S,13S,19M)-20-{5-[(3R,5S)-3,5-dimethylpiperazin-1-yl]-2-[(1S)-1-methoxyethyl]pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

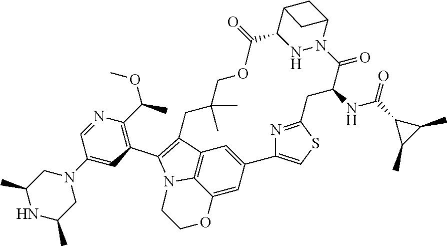

### Step 1: tert-butyl (2R,6S)-4-{5-[6-bromo-3-(3-hydroxy-2,2-dimethylpropyl)-9-oxa-1-azatricyclo[6.3.1.0^{4,12}]dodeca-2,4(12),5,7-tetraen-2-yl]-6-[(1S)-1-methoxyethyl]pyridin-3-yl}-2,6-dimethylpiperazine-1-carboxylate

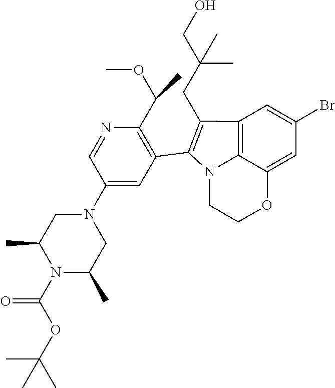 [SMILES: CO[C@@H](C)c1ncc(N2C[C@@H](C)N(C(=O)OC(C)(C)C)[C@@H](C)C2)cc1-c1c(CC(C)(C)CO)c2cc(Br)cc3c2n1CCO3]

The title compound (150 mg, 56%) was prepared in a manner similar to that in example 32 step 4 from (5-{3-[3-(acetyloxy)-2,2-dimethylpropyl]-6-bromo-9-oxa-1-azatricyclo[6.3.1.0^{4,12}]dodeca-2,4(12),5,7-tetraen-2-yl}-6-[(1S)-1-methoxyethyl]pyridin-3-yl)boronic acid and tert-butyl (2R,6S)-2,6-dimethylpiperazine-1-carboxylate. LC-MS (M+H)⁺=671.3/673.3.

### Step 2: (2-{5-[(3R,5S)-4-[(tert-butoxy)carbonyl]-3,5-dimethylpiperazin-1-yl]-2-[(1S)-1-methoxyethyl]pyridin-3-yl}-3-(3-hydroxy-2,2-dimethylpropyl)-9-oxa-1-azatricyclo[6.3.1.0^{4,12}]dodeca-2,4(12),5,7-tetraen-6-yl)boronic Acid

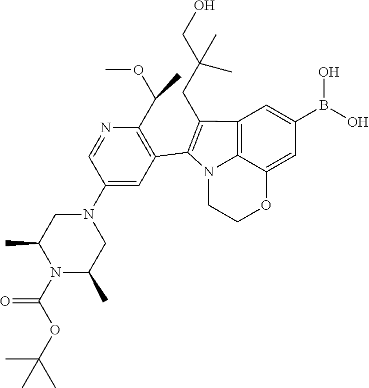 [SMILES: CO[C@@H](C)c1ncc(N2C[C@@H](C)N(C(=O)OC(C)(C)C)[C@@H](C)C2)cc1-c1c(CC(C)(C)CO)c2cc(B(O)O)cc3c2n1CCO3]

The title compound (600 mg, 20%) was prepared in a manner similar to that in example 32 step 7 from tert-butyl (2R,6S)-4-{5-[6-bromo-3-(3-hydroxy-2,2-dimethylpropyl)-9-oxa-1-azatricyclo[6.3.1.0^{4,12}]dodeca-2,4(12),5,7-tetraen-2-yl]-6-[(1S)-1-methoxyethyl]pyridin-3-yl}-2,6-dimethylpiperazine-1-carboxylate. LC-MS (M+H)⁺=637.3.

### Step 3: (4S)-2-[(2S)-3-[4-(2-{5-[(3R,5S)-4-[(tert-butoxy)carbonyl]-3,5-dimethylpiperazin-1-yl]-2-[(1S)-1-methoxyethyl]pyridin-3-yl}-3-(3-hydroxy-2,2-dimethylpropyl)-9-oxa-1-azatricyclo[6.3.1.0^{4,12}]dodeca-2,4(12),5,7-tetraen-6-yl)-1,3-thiazol-2-yl]-2-{[(1R,2R,3S)-2,3-dimethylcyclopropyl]formamido}propanoyl]-2,3-diazabicyclo[3.1.1]heptane-4-carboxylic Acid

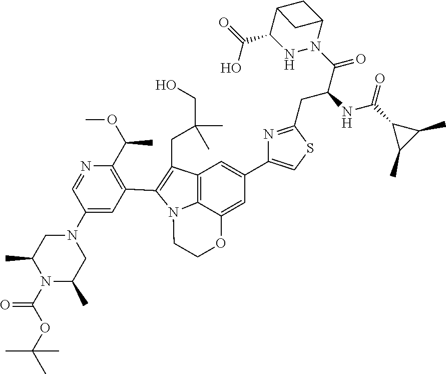 [SMILES: CO[C@@H](C)c1ncc(N2C[C@@H](C)N(C(=O)OC(C)(C)C)[C@@H](C)C2)cc1-c1c(CC(C)(C)CO)c2cc(-c3csc(C[C@H](NC(=O)[C@H]4[C@H](C)[C@@H]4C)C(=O)N4N[C@H](C(=O)O)C5CC4C5)n3)cc3c2n1CCO3]

The title compound (600 mg, 65%) was prepared in a manner similar to that in example 32 step 8 from (2-{5-[(3R,5S)-4-[(tert-butoxy)carbonyl]-3,5-dimethylpiperazin-1-yl]-2-[(1S)-1-methoxyethyl]pyridin-3-yl}-3-(3-hydroxy-2,2-dimethylpropyl)-9-oxa-1-azatricyclo[6.3.1.0^{4,12}]dodeca-2,4(12),5,7-tetraen-6-yl)boronic acid and (4S)-2-[(2S)-3-(4-bromo-1,3-thiazol-2-yl)-2-{[(1R,2R,3S)-2,3-dimethylcyclopropyl]formamido}propanoyl]-2,3-diazabicyclo[3.1.1]heptane-4-carboxylic acid. LC-MS (M+H)⁺=983.2.

### Step 4: tert-butyl (2R,6S)-4-{5-[(7S,13S)-7-[(1R,2R,3S)-2,3-dimethylcyclopropaneamido]-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-20-yl]-6-[(1S)-1-methoxyethyl]pyridin-3-yl}-2,6-dimethylpiperazine-1-carboxylate

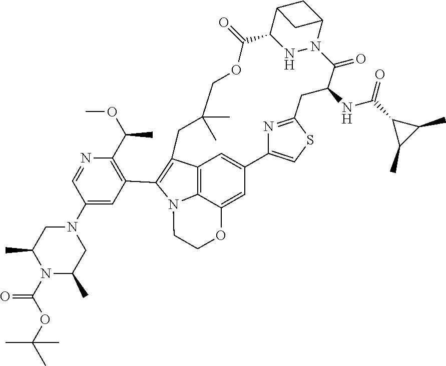 [SMILES: CO[C@@H](C)c1ncc(N2C[C@@H](C)N(C(=O)OC(C)(C)C)[C@@H](C)C2)cc1-c1c2c3cc(cc4c3n1CCO4)-c1csc(n1)C[C@H](NC(=O)[C@H]1[C@H](C)[C@@H]1C)C(=O)N1N[C@H](C(=O)OCC(C)(C)C2)C2CC1C2]

The title compound (320 mg, 54%) was prepared in a manner similar to that in example 32 step 9 from (4S)-2-[(2S)-3-[4-(2-{5-[(3R,5S)-4-[(tert-butoxy)carbonyl]-3,5-dimethylpiperazin-1-yl]-2-[(1S)-1-methoxyethyl]pyridin-3-yl}-3-(3-hydroxy-2,2-dimethylpropyl)-9-oxa-1-azatricyclo[6.3.1.0^{4,12}]dodeca-2,4(12),5,7-tetraen-6-yl)-1,3-thiazol-2-yl]-2-{[(1R,2R,3S)-2,3-dimethylcyclopropyl]formamido}propanoyl]-2,3-diazabicyclo[3.1.1]heptane-4-carboxylic acid. LC-MS (M+H)⁺=965.2.

### Step 5: (1R,2R,3S)—N-[(7S,13S,19M)-20-{5-[(3R,5S)-3,5-dimethylpiperazin-1-yl]-2-[(1S)-1-methoxyethyl]pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

To a round-bottom flask charged with tert-butyl (2R,6S)-4-{5-[(7S,13S)-7-[(1R,2R,3S)-2,3-dimethylcyclopropaneamido]-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-20-yl]-6-[(1S)-1-methoxyethyl]pyridin-3-yl}-2,6-dimethylpiperazine-1-carboxylate (320 mg, 0.33 mmol) was added HCl/dioxane (4M, 10 mL). The reaction mixture was stirred at room temperature for 2 h. Solvent was removed in vacuo and the residue was diluted with saturated NaHCO₃ (5 mL) and EA (10 mL). The layer was separated, and the aqueous phase was extracted with EA (10 mL). The combined organic phase was dried over Na₂SO₄, evaporated in vacuo. The residue was purified by prep-TLC (DCM/MeOH=10:1) and then by prep-HPLC to give example 44 (200 mg, 70%). ¹H NMR (400 MHz, DMSO-d6) δ 8.41-8.32 (m, 2H), 8.00 (s, 1H), 7.73 (s, 1H), 7.11 (s, 2H), 5.86 (d, J=11.0 Hz, 1H), 5.30 (t, J=8.0 Hz, 1H), 4.66 (d, J=11.1 Hz, 1H), 4.59-4.52 (m, 2H), 4.45 (d, J=4.6 Hz, 1H), 4.30-4.21 (m, 2H), 3.65-3.62 (m, 3H), 3.51-3.48 (m, 2H), 3.23-2.11 (m, 4H), 3.13-3.05 (m, 1H), 2.90-2.73 (m, 3H), 2.61 (d, J=5.9 Hz, 1H), 2.29 (d, J=5.6 Hz, 2H), 2.18-2.07 (m, 4H), 1.54 (t, J=9.2 Hz, 1H), 1.36 (s, 1H), 1.30 (d, J=6.1 Hz, 3H), 1.18 (d, J=6.1 Hz, 1H), 1.12 (d, J=4.3 Hz, 2H), 1.03 (dd, J=11.1, 5.7 Hz, 5H), 0.96 (dd, J=6.0, 2.2 Hz, 8H), 0.88 (s, 3H), 0.33 (s, 3H). LC-MS (M+H)⁺=865.2.

### Example 45: (1R,2R,3S)—N-[(7S,13S,19M)-20-{2-[(1S)-1-methoxyethyl]-5-(2-methyl-2,6-diazaspiro[3.4]octan-6-yl)pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

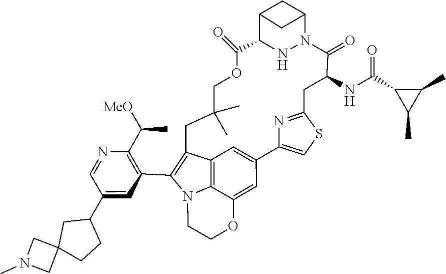

### Step 1: 3-(6-bromo-2-{2-[(1S)-1-methoxyethyl]-5-{2-methyl-2,6-diazaspiro[3.4]octan-6-yl}pyridin-3-yl}-1-azatricyclo[6.3.1.0^{4,12}]dodeca-2,4(12),5,7-tetraen-3-yl)-2,2-dimethylpropyl Acetate

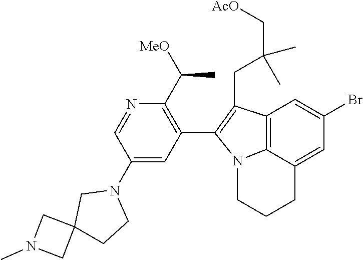 [SMILES: CO[C@@H](C)c1ncc(N2CCC3(CN(C)C3)C2)cc1-c1c(CC(C)(C)COC(C)=O)c2cc(Br)cc3c2n1CCC3]

To a mixture of 3-(6-bromo-2-{2-[(1S)-1-methoxyethyl]-5-(4,4,5,5-tetramethyl-1,3,2-dioxaborolan-2-yl)pyridin-3-yl}-1-azatricyclo[6.3.1.0^{4,12}]dodeca-2,4(12),5,7-tetraen-3-yl)-2,2-dimethylpropyl acetate (1 g, 1.6 mmol) and 2-ethyl-2,6-diazaspiro[3.4]octane (1 g, 8 mmol) in MeCN (10 mL) was added TEA (0.48 g, 4.8 mmol) and Cu(OAc)₂ (0.58 g, 3.2 mmol). The mixture was stirred for 4 h at room temperature under oxygen atmosphere. The mixture was then diluted with EA (50 mL) and water (50 mL). The solid was filtered off and the filter cake was rinsed with EA (2×50 mL). The organic phase was separated and washed with water (50 mL), brine (50 mL), dried over Na₂SO₄, filtered and concentrated under vacuum. The residue was purified by silica gel column chromatography (DCM:MeOH=15:1) to give the title compound (0.32 g, 32%). LC-MS (M+H)⁺=623.3/625.3.

### Step 2: 3-(2-{2-[(1S)-1-methoxyethyl]-5-{2-methyl-2,6-diazaspiro[3.4]octan-6-yl}pyridin-3-yl}-6-(4,4,5,5-tetramethyl-1,3,2-dioxaborolan-2-yl)-1-azatricyclo[6.3.1.0^{4,12}]dodeca-2,4(12),5,7-tetraen-3-yl)-2,2-dimethylpropyl Acetate

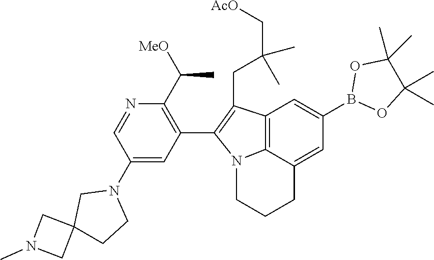

To a mixture of 3-(6-bromo-2-{2-[(1S)-1-methoxyethyl]-5-{2-methyl-2,6-diazaspiro[3.4]octan-6-yl}pyridin-3-yl}-1-azatricyclo[6.3.1.0^{4,12}]dodeca-2,4(12),5,7-tetraen-3-yl)-2,2-dimethylpropyl acetate (320 mg, 0.51 mmol) and BPD (327 mg, 1.28 mmol) in dioxane (10 mL) was added Pd(dppf)Cl₂·DCM (32 mg, 0.039 mmol) and KOAc (142 mg, 1.45 mmol). The mixture was stirred for 4 h at 90° C. under nitrogen. The mixture was cooled to room temperature and then diluted with EA (40 mL), washed with brine (20 mL). The organic phase was dried over Na₂SO₄, filtered and concentrated under vacuum. The residue was purified by prep-TLC (DCM:MeOH=15:1) to give the title compound (190 mg, 55%). LC-MS (M+H)⁺=671.5.

### Step 3: methyl (4S)-2-[(2S)-3-(4-{3-[3-(acetyloxy)-2,2-dimethylpropyl]-2-{2-[(1S)-1-methoxyethyl]-5-{2-methyl-2,6-diazaspiro[3.4]octan-6-yl}pyridin-3-yl}-1-azatricyclo[6.3.1.0^{4,12}]dodeca-2,4(12),5,7-tetraen-6-yl}-1,3-thiazol-2-yl)-2-{[(1R,2R,3S)-2,3-dimethylcyclopropyl]formamido}propanoyl]-2,3-diazabicyclo[3.1.1]heptane-4-carboxylate

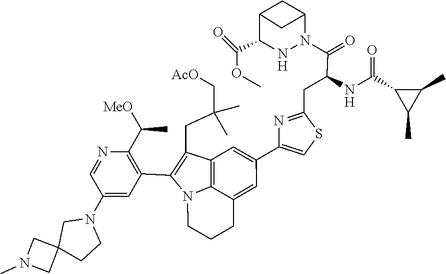

To a mixture of 3-(2-{2-[(1S)-1-methoxyethyl]-5-{2-methyl-2,6-diazaspiro[3.4]octan-6-yl}pyridin-3-yl}-6-(4,4,5,5-tetramethyl-1,3,2-dioxaborolan-2-yl)-1-azatricyclo[6.3.1.0^{4,12}]dodeca-2,4(12),5,7-tetraen-3-yl)-2,2-dimethylpropyl acetate (190 mg, 0.28 mmol) and methyl (4S)-2-[(2S)-3-(4-bromo-1,3-thiazol-2-yl)-2-{[(1R,2R,3S)-2,3-dimethylcyclopropyl]formamido}propanoyl]-2,3-diazabicyclo[3.1.1]heptane-4-carboxylate (138 mg, 0.283 mmol) in dioxane (10 mL) and water (1 mL) was added K₃PO₄ (180 mg, 0.85 mmol) and Pd(dtbpf)Cl₂ (13 mg, 0.02 mmol). The solution was stirred for 3 h at 70° C. under nitrogen and then cooled to room temperature. The mixture was diluted with EA (30 mL) and washed with water (30 mL) and brine (30 mL). The organic phase was dried over Na₂SO₄, filtered and concentrated under vacuum. The residue was purified by silica gel column chromatography (DCM:MeOH=15:1) to give the title compound (250 mg, 93%). LC-MS (M+H)⁺=949.5.

### Step 4: (4S)-2-[(2S)-2-{[(1R,2R,3S)-2,3-dimethylcyclopropyl]formamido}-3-{4-[3-(3-hydroxy-2,2-dimethylpropyl)-2-{2-[(1S)-1-methoxyethyl]-5-{2-methyl-2,6-diazaspiro[3.4]octan-6-yl}pyridin-3-yl}-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic Acid

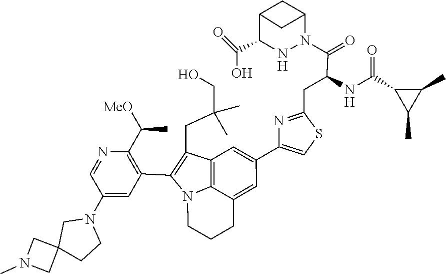

To a solution of methyl (4S)-2-[(2S)-3-(4-{3-[3-(acetyloxy)-2,2-dimethylpropyl]-2-{2-[(1S)-1-methoxyethyl]-5-{2-methyl-2,6-diazaspiro[3.4]octan-6-yl}pyridin-3-yl}-1-azatricyclo[6.3.1.0^{4,12}]dodeca-2,4(12),5,7-tetraen-6-yl}-1,3-thiazol-2-yl)-2-{[(R,2R,3S)-2,3-dimethylcyclopropyl]formamido}propanoyl]-2,3-diazabicyclo[3.1.1]heptane-4-carboxylate (250 mg, 0.26 mmol) in THF (10 mL) and water (10 mL) was added LiOH·H₂O (55 mg, 1.3 mmol). The resulting mixture was stirred for 6 h at room temperature. The mixture was diluted with water (10 mL) and washed with EA (10 mL). The aqueous phase was acidified to pH ˜5 with 1 N hydrochloric acid at 0° C. The mixture was extracted with DCM/MeOH (10/1, 3×20 mL). The organic phases were combined, dried over Na₂SO₄, filtered and concentrated under vacuum to give the title compound (120 mg, 52%). LC-MS (M+H)⁺=893.5.

### Step 5: (1R,2R,3S)—N-[(7S,13S,19M)-20-{2-[(1S)-1-methoxyethyl]-5-{2-methyl-2,6-diazaspiro[3.4]octan-6-yl}pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

To a solution of (4S)-2-[(2S)-2-{[(R,2R,3S)-2,3-dimethylcyclopropyl]formamido)-3-{4-[3-(3-hydroxy-2,2-dimethylpropyl)-2-{2-[(1S)-1-methoxyethyl]-5-(2-methyl-2,6-diazaspiro[3.4]octan-6-yl}pyridin-3-yl}-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic acid (120 mg, 0.13 mmol) in dry DCM (12 mL) was added DIEA (138 mg, 1.07 mmol), EDCI (129 mg, 0.67 mmol) and HOBT (54 mg, 0.4 mmol) and DMAP (8.2 mg, 0.067 mmol). The mixture was stirred for 16 h at room temperature and then diluted with DCM (10 mL), washed with brine (10 mL). The organic phase was dried over Na₂SO₄, filtered, and concentrated under vacuum. The residue was purified by prep-TLC (DCM:MeOH=10:1) and then by prep-HPLC to give example 45 (M-isomer, 2 mg, 1.7%). ¹H NMR (400 MHz, DMSO-d6) δ 8.37 (d, J=8.9 Hz, 1H), 8.20 (s, 1H), 8.01 (d, J=2.8 Hz, 1H), 7.68 (s, 1H), 7.39 (s, 1H), 6.62 (d, J=2.7 Hz, 1H), 5.88 (d, J=11.1 Hz, 1H), 5.37-5.27 (m, 1H), 4.64 (d, J=11.0 Hz, 1H), 4.48-4.38 (m, 1H), 4.24-4.11 (m, 2H), 3.59-3.36 (m, 5H), 3.25-3.14 (m, 6H), 3.12-3.03 (m, 5H), 3.0-2.83 (m, 3H), 2.66-2.56 (m, 2H), 2.42-2.37 (m, 1H), 2.29-2.26 (m, 1H), 2.17 (s, 3H), 2.11-2.05 (m, 3H), 1.56-1.5 (m, 1H), 1.28 (d, J=6.1 Hz, 3H), 1.22-0.98 (m, 11H), 0.86 (s, 3H), 0.36 (s, 3H). LC-MS (M+H)⁺=875.5.

### Example 46: (1R,2R,3S)—N-[(7S,13S,19M)-20-[5-(4-ethylpiperazin-1-yl)-2-[(1S)-1-methoxyethyl]pyridin-3-yl]-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

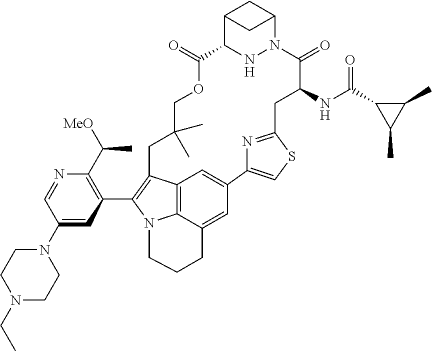

### Step 1: 3-{6-bromo-2-[5-(4-ethylpiperazin-1-yl)-2-[(1S)-1-methoxyethyl]pyridin-3-yl]-1-azatricyclo[6.3.1.0^{4,12}]dodeca-2,4(12),5,7-tetraen-3-yl}-2,2-dimethylpropyl Acetate

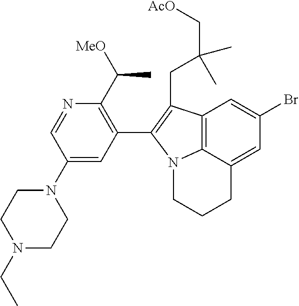 [SMILES: CCN1CCN(c2cnc([C@H](C)OC)c(-c3c(CC(C)(C)COC(C)=O)c4cc(Br)cc5c4n3CCC5)c2)CC1]

The title compound (0.4 g, 41%) was prepared in a manner similar to that in example 1 step 10 from 3-(6-bromo-2-{2-[(1S)-1-methoxyethyl]-5-(4,4,5,5-tetramethyl-1,3,2-dioxaborolan-2-yl)pyridin-3-yl}-1-azatricyclo[6.3.1.0^{4,12}]dodeca-2,4(12),5,7-tetraen-3-yl)-2,2-dimethylpropyl acetate and 1-ethyl piperazine. LC-MS (M+H)⁺=611.3/613.3.

### Step 2: 3-{2-[5-(4-ethylpiperazin-1-yl)-2-[(1S)-1-methoxyethyl]pyridin-3-yl]-6-(4,4,5,5-tetramethyl-1,3,2-dioxaborolan-2-yl)-1-azatricyclo[6.3.1.0^{4,12}]dodeca-2,4(12),5,7-tetraen-3-yl}-2,2-dimethylpropyl Acetate

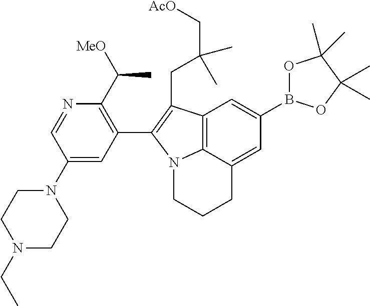 [SMILES: CCN1CCN(c2cnc([C@H](C)OC)c(-c3c(CC(C)(C)COC(C)=O)c4cc(B5OC(C)(C)C(C)(C)O5)cc5c4n3CCC5)c2)CC1]

The title compound (0.27 g, 63%) was prepared in a manner similar to that in example 1 step 11 from 3-{6-bromo-2-[5-(4-ethylpiperazin-1-yl)-2-[(1S)-1-methoxyethyl]pyridin-3-yl]-1-azatricyclo[6.3.1.0^{4,12}]dodeca-2,4(12),5,7-tetraen-3-yl}-2,2-dimethylpropyl acetate and BPD. LC-MS (M+H)⁺=659.3.

### Step 3: methyl (4S)-2-[(2S)-3-(4-{3-[3-(acetyloxy)-2,2-dimethylpropyl]-2-[5-(4-ethylpiperazin-1-yl)-2-[(1S)-1-methoxyethyl]pyridin-3-yl]-1-azatricyclo[6.3.1.0^{4,12}]dodeca-2,4(12),5,7-tetraen-6-yl}-1,3-thiazol-2-yl)-2-{[(1R,2R,3S)-2,3-dimethylcyclopropyl]formamido}propanoyl]-2,3-diazabicyclo[3.1.1]heptane-4-carboxylate

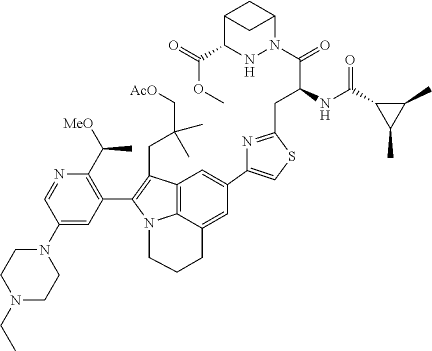 [SMILES: CCN1CCN(c2cnc([C@H](C)OC)c(-c3c(CC(C)(C)COC(C)=O)c4cc(-c5csc(C[C@H](NC(=O)[C@H]6[C@H](C)[C@@H]6C)C(=O)N6N[C@H](C(=O)OC)C7CC6C7)n5)cc5c4n3CCC5)c2)CC1]

The title compound (0.26 g, 68%) was prepared in a manner similar to that in example 35 step 4 from 3-{2-[5-(4-ethylpiperazin-1-yl)-2-[(1S)-1-methoxyethyl]pyridin-3-yl]-6-(4,4,5,5-tetramethyl-1,3,2-dioxaborolan-2-yl)-1-azatricyclo[6.3.1.0^{4,12}]dodeca-2,4(12),5,7-tetraen-3-yl}-2,2-dimethylpropyl acetate and methyl (4S)-2-[(2S)-3-(4-bromo-1,3-thiazol-2-yl)-2-{[(1R,2R,3S)-2,3-dimethylcyclopropyl]formamido}propanoyl]-2,3-diazabicyclo[3.1.1]heptane-4-carboxylate. LC-MS (M+H)⁺=937.3.

### Step 4: (4S)-2-[(2S)-2-{[(1R,2R,3S)-2,3-dimethylcyclopropyl]formamido}-3-(4-{2-[5-(4-ethylpiperazin-1-yl)-2-[(1S)-1-methoxyethyl]pyridin-3-yl]-3-(3-hydroxy-2,2-dimethylpropyl)-1-azatricyclo[6.3.1.0^{4,12}]dodeca-2,4(12),5,7-tetraen-6-yl}-1,3-thiazol-2-yl)propanoyl]-2,3-diazabicyclo[3.1.1]heptane-4-carboxylic Acid

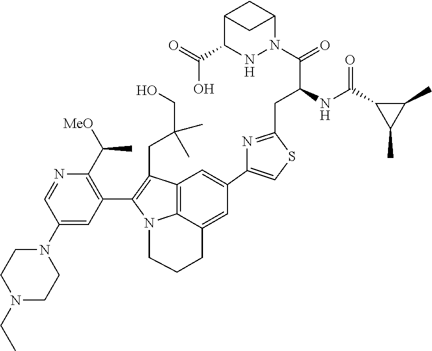 [SMILES: CCN1CCN(c2cnc([C@H](C)OC)c(-c3c(CC(C)(C)CO)c4cc(-c5csc(C[C@H](NC(=O)[C@H]6[C@H](C)[C@@H]6C)C(=O)N6N[C@H](C(=O)O)C7CC6C7)n5)cc5c4n3CCC5)c2)CC1]

The title compound (0.17 g, 70%) was prepared in a manner similar to that in example 35 step 5 from methyl (4S)-2-[(2S)-3-(4-{3-[3-(acetyloxy)-2,2-dimethylpropyl]-2-[5-(4-ethylpiperazin-1-yl)-2-[(1S)-1-methoxyethyl]pyridin-3-yl]-1-azatricyclo[6.3.1.0^{4,12}]dodeca-2,4(12),5,7-tetraen-6-yl}-1,3-thiazol-2-yl)-2-{[(1R,2R,3S)-2,3-dimethylcyclopropyl]formamido}propanoyl]-2,3-diazabicyclo[3.1.1]heptane-4-carboxylate. LC-MS (M+H)⁺=881.3.

### Step 5: (1R,2R,3S)—N-[(7S,13S,19M)-20-[5-(4-ethylpiperazin-1-yl)-2-[(1S)-1-methoxyethyl]pyridin-3-yl]-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

Example 46 (M-isomer, 10 mg, 6%) was prepared in a manner similar to that in example 16 step 7 from (4S)-2-[(2S)-2-{[(R,2R,3S)-2,3-dimethylcyclopropyl]formamido}-3-(4-{2-[5-(4-ethylpiperazin-1-yl)-2-[(1S)-1-methoxyethyl]pyridin-3-yl]-3-(3-hydroxy-2,2-dimethylpropyl)-1-azatricyclo[6.3.1.0^{4,12}]dodeca-2,4(12),5,7-tetraen-6-yl}-1,3-thiazol-2-yl)propanoyl]-2,3-diazabicyclo[3.1.1]heptane-4-carboxylic acid ¹H NMR (400 MHz, DMSO-d6) δ 8.43-8.33 (m, 2H), 8.20 (s, 1H), 7.67 (s, 1H), 7.39 (s, 1H), 7.10 (d, J=2.6 Hz, 1H), 5.88 (d, J=11.0 Hz, 1H), 5.38-5.29 (m, 1H), 4.63 (d, J=11.0 Hz, 1H), 4.48-4.41 (m, 1H), 4.27-4.11 (m, 2H), 3.61-3.42 (m, 3H), 3.24-3.14 (m, 8H), 3.13-2.80 (m, 4H), 2.63-2.56 (m, 1H), 2.44-2.37 (m, 6H), 2.35-2.25 (m, 3H), 2.22-2.02 (m, 3H), 1.55-1.46 (m, 1H), 1.29 (d, J=6.0 Hz, 3H), 1.23-1.1 (m, 3H), 1.07-0.93 (m, 9H), 0.85 (s, 3H), 0.34 (s, 3H). LC-MS (M+H)⁺=863.4.

### Example 47: (1R,2R,3S)—N-[(7S,13S,19M)-20-{5-[(3aR,6aS)-5-methyl-octahydropyrrolo[3,4-c]pyrrol-2-yl]-2-[(1S)-1-methoxyethyl]pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

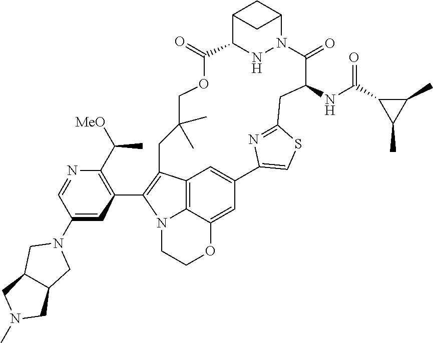

### Step 1: tert-butyl (3aR,6aS)-5-(5-{3-[3-(acetyloxy)-2,2-dimethylpropyl]-6-bromo-9-oxa-1-azatricyclo[6.3.1.0^{4,12}]dodeca-2,4(12),5,7-tetraen-2-yl}-6-[(1S)-1-methoxyethyl]pyridin-3-yl)-octahydropyrrolo[3,4-c]pyrrole-2-carboxylate

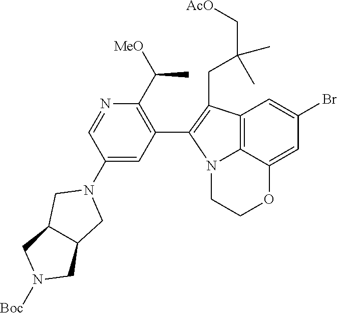 [SMILES: CO[C@@H](C)c1ncc(N2C[C@H]3CN(C(=O)OC(C)(C)C)C[C@H]3C2)cc1-c1c(CC(C)(C)COC(C)=O)c2cc(Br)cc3c2n1CCO3]

To a mixture of (5-{3-[3-(acetyloxy)-2,2-dimethylpropyl]-6-bromo-9-oxa-1-azatricyclo[6.3.1.0^{4,12}]dodeca-2,4(12),5,7-tetraen-2-yl}-6-[(1S)-1-methoxyethyl]pyridin-3-yl)boronic acid (0.5 g, 0.92 mmol) and tert-butyl (3aR,6aS)-hexahydropyrrolo[3,4-c]pyrrole-2(1H)-carboxylate (0.39 g, 1.84 mmol) in MeCN (10 mL) was added TEA (0.28 g, 2.76 mmol) and Cu(OAc)₂ (0.33 g, 1.84 mmol). The mixture was stirred for 4 h at room temperature under oxygen atmosphere. The mixture was then diluted with EA (30 mL) and water (30 mL). The solid was filtered off and the filter cake was rinsed with EA (2×50 mL). The organic phase was separated and washed with water (50 mL), brine (50 mL), dried over Na₂SO₄, filtered and concentrated under vacuum. The residue was purified by silica gel column chromatography (DCM:MeOH=15:1) to give the title compound (0.3 g, 45%). LC-MS (M+H)⁺=711.3/713.3.

### Step 2: 3-(2-{5-[(3aR,6aS)-octahydropyrrolo[3,4-c]pyrrol-2-yl]-2-[(1S)-1-methoxyethyl]pyridin-3-yl}-6-bromo-9-oxa-1-azatricyclo[6.3.1.0^{4,12}]dodeca-2,4(12),5,7-tetraen-3-yl)-2,2-dimethylpropan-1-ol

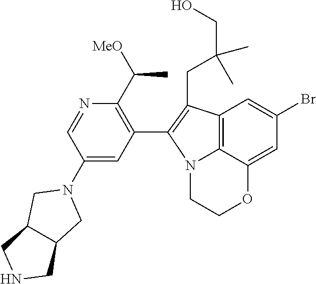 [SMILES: CO[C@@H](C)c1ncc(N2C[C@H]3CNC[C@H]3C2)cc1-c1c(CC(C)(C)CO)c2cc(Br)cc3c2n1CCO3]

A mixture of tert-butyl (3aR,6aS)-5-(5-{3-[3-(acetyloxy)-2,2-dimethylpropyl]-6-bromo-9-oxa-1-azatricyclo[6.3.1.0^{4,12}]dodeca-2,4(12),5,7-tetraen-2-yl}-6-[(1S)-1-methoxyethyl]pyridin-3-yl)-octahydropyrrolo[3,4-c]pyrrole-2-carboxylate (0.3 g, 0.1 mmol) in HCl/dioxane (4 M, 10 mL) was stirred for 3 h at room temperature and then concentrated under vacuum. The residue was diluted with DCM (50 mL), washed with saturated NaHCO₃ (50 mL), dried over Na₂SO₄, filtered, and concentrated under vacuum to give the title compound (0.15 g, 62%). LC-MS (M+H)⁺=569.3/571.3.

### Step 3: 3-(2-{5-[(3aR,6aS)-5-methyl-octahydropyrrolo[3,4-c]pyrrol-2-yl]-2-[(1S)-1-methoxyethyl]pyridin-3-yl}-6-bromo-9-oxa-1-azatricyclo[6.3.1.0^{4,12}]dodeca-2,4(12),5,7-tetraen-3-yl)-2,2-dimethylpropan-1-ol

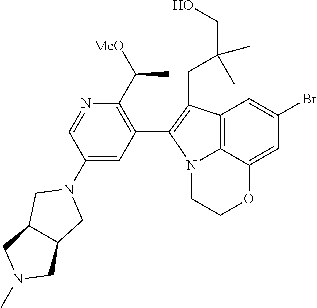 [SMILES: CO[C@@H](C)c1ncc(N2C[C@H]3CN(C)C[C@H]3C2)cc1-c1c(CC(C)(C)CO)c2cc(Br)cc3c2n1CCO3]

To a solution of 3-(2-{5-[(3aR,6aS)-octahydropyrrolo[3,4-c]pyrrol-2-yl]-2-[(1S)-1-methoxyethyl]pyridin-3-yl}-6-bromo-9-oxa-1-azatricyclo[6.3.1.0^{4,12}]dodeca-2,4(12),5,7-tetraen-3-yl)-2,2-dimethylpropan-1-ol (0.15 g, 0.26 mmol) in DCM (6 mL) was 37% HCHO (82 mg, 1 mmol) and NaBH(OAc)₃ (110 mg, 0.52 mmol) at room temperature. The reaction was stirred at room temperature for 1 h. The mixture was diluted with DCM (20 mL), washed with NaHCO₃ (20 mL) and brine (20 mL). The organic phase was dried over Na₂SO₄, filtered and concentrated under vacuum. The residue was purified by silica gel column chromatography (DCM:MeOH=15:1) to give the title compound (0.12 g, 80%). LC-MS (M+H)⁺=583.3/585.3.

### Step 4: 3-(2-{5-[(3aR,6aS)-5-methyl-octahydropyrrolo[3,4-c]pyrrol-2-yl]-2-[(1S)-1-methoxyethyl]pyridin-3-yl}-6-(4,4,5,5-tetramethyl-1,3,2-dioxaborolan-2-yl)-9-oxa-1-azatricyclo[6.3.1.0^{4,12}]dodeca-2,4(12),5,7-tetraen-3-yl)-2,2-dimethylpropan-1-ol

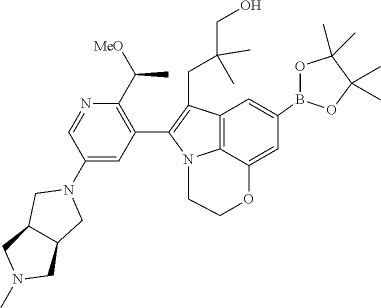

To a mixture of 3-(2-{5-[(3aR,6aS)-5-methyl-octahydropyrrolo[3,4-c]pyrrol-2-yl]-2-[(1S)-1-methoxyethyl]pyridin-3-yl}-6-bromo-9-oxa-1-azatricyclo[6.3.1.0^{4,12}]dodeca-2,4(12),5,7-tetraen-3-yl)-2,2-dimethylpropan-1-ol (120 mg, 0.2 mmol) and BPD (152 mg, 0.6 mmol) in dioxane (10 mL) was added Pd(dppf)Cl₂·DCM (16 mg, 0.02 mmol) and KOAc (59 mg, 0.6 mmol). The mixture was stirred for 4 h at 90° C. under nitrogen. The mixture was cooled to room temperature and then diluted with EA (40 mL), washed with brine (20 mL). The organic phase was dried over Na₂SO₄, filtered and concentrated under vacuum. The residue was purified by prep-TLC (DCM:MeOH=15:1) to give the title compound (120 mg, 92%). LC-MS (M+H)⁺=631.5.

### Step 5: methyl (4S)-2-[(2S)-3-[4-(2-{5-[(3aR,6aS)-5-methyl-octahydropyrrolo[3,4-c]pyrrol-2-yl]-2-[(1S)-1-methoxyethyl]pyridin-3-yl}-3-(3-hydroxy-2,2-dimethylpropyl)-9-oxa-1-azatricyclo[6.3.1.0^{4,12}]dodeca-2,4(12),5,7-tetraen-6-yl)-1,3-thiazol-2-yl]-2-{[(1R,2R,3S)-2,3-dimethylcyclopropyl]formamido}propanoyl]-2,3-diazabicyclo[3.1.1]heptane-4-carboxylate

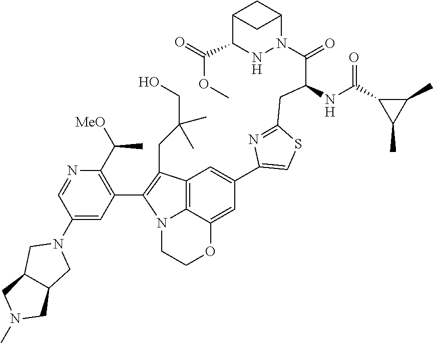

To a mixture of 3-(2-{5-[(3aR,6aS)-5-methyl-octahydropyrrolo[3,4-c]pyrrol-2-yl]-2-[(1S)-1-methoxyethyl]pyridin-3-yl}-6-(4,4,5,5-tetramethyl-1,3,2-dioxaborolan-2-yl)-9-oxa-1-azatricyclo[6.3.1.0^{4,12}]dodeca-2,4(12),5,7-tetraen-3-yl)-2,2-dimethylpropan-1-ol (120 mg, 0.19 mmol) and methyl (4S)-2-[(2S)-3-(4-bromo-1,3-thiazol-2-yl)-2-{[(R,2R,3S)-2,3-dimethylcyclopropyl]formamido}propanoyl]-2,3-diazabicyclo[3.1.1]heptane-4-carboxylate (97 mg, 0.2 mmol) in dioxane (10 mL) and water (3 mL) was added K₃PO₄ (127 mg, 0.6 mmol) and Pd(dtbpf)Cl₂ (13 mg, 0.02 mmol). The solution was stirred for 3 h at 70° C. under nitrogen and then cooled to room temperature. The mixture was diluted with EA (20 mL), washed with water (10 mL), brine (10 mL). The organic phase was dried over Na₂SO₄, filtered and concentrated under vacuum. The residue was purified by silica gel column chromatography (DCM:MeOH=15:1) to give the title compound (150 mg, 76%). LC-MS (M+H)⁺=909.5.

### Step 6: (4S)-2-[(2S)-3-[4-(2-{5-[(3aR,6aS)-5-methyl-octahydropyrrolo[3,4-c]pyrrol-2-yl]-2-[(1S)-1-methoxyethyl]pyridin-3-yl}-3-(3-hydroxy-2,2-dimethylpropyl)-9-oxa-1-azatricyclo[6.3.1.0^{4,12}]dodeca-2,4(12),5,7-tetraen-6-yl)-1,3-thiazol-2-yl]-2-{[(1R,2R,3S)-2,3-dimethylcyclopropyl]formamido}propanoyl]-2,3-diazabicyclo[3.1.1]heptane-4-carboxylic Acid

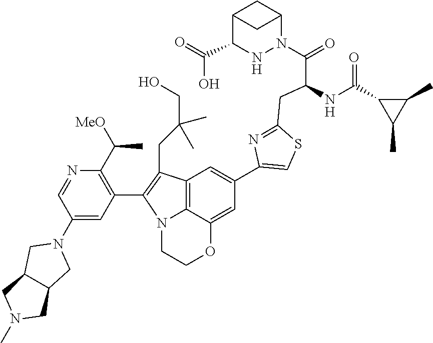

To a solution of methyl (4S)-2-[(2S)-3-[4-(2-{5-[(3aR,6aS)-5-methyl-octahydropyrrolo[3,4-c]pyrrol-2-yl]-2-[(1S)-1-methoxyethyl]pyridin-3-yl}-3-(3-hydroxy-2,2-dimethylpropyl)-9-oxa-1-azatricyclo[6.3.1.0^{4,12}]dodeca-2,4(12),5,7-tetraen-6-yl)-1,3-thiazol-2-yl]-2-{[(1R,2R,3S)-2,3-dimethylcyclopropyl]formamido}propanoyl]-2,3-diazabicyclo[3.1.1]heptane-4-carboxylate (150 mg, 0.16 mmol) in THF (5 mL) and water (5 mL) was added LiOH·H₂O (20 mg, 0.48 mmol). The resulting mixture was stirred for 2 h at room temperature. The mixture was diluted with water (20 mL) and washed with EA (10 mL). The aqueous phase was acidified to pH ˜5 with 1 N hydrochloric acid at 0° C. The mixture was extracted with DCM/MeOH (10/1,3×20 mL). The organic phases were combined, dried over Na₂SO₄, filtered and concentrated under vacuum to give the title compound (90 mg, 63%). LC-MS (M+H)⁺=895.5.

### Step 7: (1R,2R,3S)—N-[(7S,13S,19M)-20-{5-[(3aR,6aS)-5-methyl-octahydropyrrolo[3,4-c]pyrrol-2-yl]-2-[(1S)-1-methoxyethyl]pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

To a solution of (4S)-2-[(2S)-3-[4-(2-{5-[(3aR,6aS)-5-methyl-octahydropyrrolo[3,4-c]pyrrol-2-yl]-2-[(1S)-1-methoxyethyl]pyridin-3-yl}-3-(3-hydroxy-2,2-dimethylpropyl)-9-oxa-1-azatricyclo[6.3.1.0^{4,12}]dodeca-2,4(12),5,7-tetraen-6-yl)-1,3-thiazol-2-yl]-2-{[(1R,2R,3S)-2,3-dimethylcyclopropyl]formamido}propanoyl]-2,3-diazabicyclo[3.1.1]heptane-4-carboxylic acid (90 mg, 0.1 mmol) in dry DCM (10 mL) was added DIEA (69 mg, 0.5 mmol), EDCI (64 mg, 0.33 mmol), HOBT (27 mg, 0.2 mmol) and DMAP (4.2 mg, 0.033 mmol). The mixture was stirred for 16 h at room temperature and then diluted with DCM (10 mL), washed with brine (10 mL). The organic phase was dried over Na₂SO₄, filtered and concentrated under vacuum. The residue was purified by prep-TLC (DCM:MeOH=10:1) and then by prep-HPLC to give example 47 (M-isomer, 3 mg, 3.4%). ¹H NMR (400 MHz, DMSO-d6) δ 8.42-8.3 (m, 2H), 8.02 (s, 1H), 7.72 (s, 1H), 7.11 (s, 1H), 6.99 (s, 1H), 5.83 (d, J=10.8 Hz, 1H), 5.26 (t, J=8.0 Hz, 1H), 4.69 (d, J=10.9 Hz, 1H), 4.52-4.32 (m, 3H), 4.06-3.98 (m, 1H), 3.96-3.88 (m, 1H), 3.66-3.54 (m, 3H), 3.53-3.44 (m, 3H), 3.17-3.12 (m, 2H), 3.12-2.92 (m, 7H), 2.89-2.82 (m, 2H), 2.66-2.59 (m, 2H), 2.41-2.26 (m, 4H), 2.20-2.04 (m, 4H), 1.62-1.53 (m, 1H), 1.24-0.97 (m, 14H), 0.90 (s, 3H), 0.49 (s, 3H). LC-MS (M+H)⁺=877.4.

### Example 48: (1R,2R,3S)—N-[(7S,13S,19M)-20-{2-[(1S)-1-methoxyethyl]-5-{2-oxa-6-azaspiro[3.4]octan-6-yl}pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

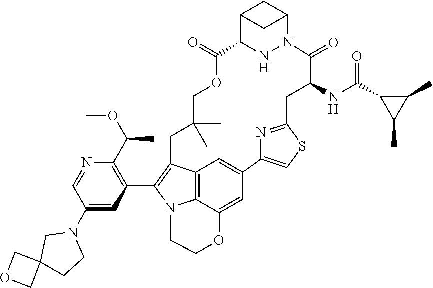

### Step 1: 3-(6-bromo-2-{2-[(1S)-1-methoxyethyl]-5-{2-oxa-6-azaspiro[3.4]octan-6-yl}pyridin-3-yl}-9-oxa-1-azatricyclo[6.3.1.0^{4,12}]dodeca-2,4(12),5,7-tetraen-3-yl)-2,2-dimethylpropan-1-ol

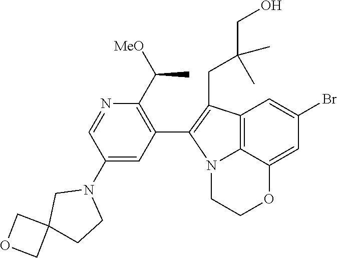 [SMILES: CO[C@@H](C)c1ncc(N2CCC3(COC3)C2)cc1-c1c(CC(C)(C)CO)c2cc(Br)cc3c2n1CCO3]

The title compound (0.14 g, 13%) was prepared in a manner similar to that in example 9 step 2 from {5-[6-bromo-3-(3-hydroxy-2,2-dimethylpropyl)-9-oxa-1-azatricyclo[6.3.1.0^{4,12}]dodeca-2,4(12),5,7-tetraen-2-yl]-6-[(1S)-1-methoxyethyl]pyridin-3-yl}boronic acid and 2-oxa-6-azaspiro[3.4]octane. LC-MS (M+H)⁺=570.4/572.4.

### Step 2: [3-(3-hydroxy-2,2-dimethylpropyl)-2-{2-[(1S)-1-methoxyethyl]-5-{2-oxa-6-azaspiro[3.4]octan-6-yl}pyridin-3-yl}-9-oxa-1-azatricyclo[6.3.1.0^{4,12}]dodeca-2,4(12),5,7-tetraen-6-yl]boronic Acid

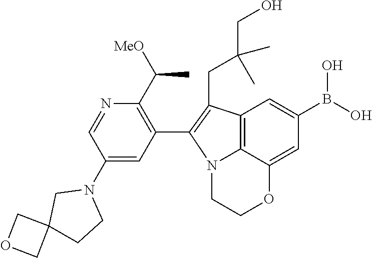 [SMILES: CO[C@@H](C)c1ncc(N2CCC3(COC3)C2)cc1-c1c(CC(C)(C)CO)c2cc(B(O)O)cc3c2n1CCO3]

The title compound (110 mg, 82%) was prepared in a manner similar to that in example 32 step 7 from 3-(6-bromo-2-{2-[(1S)-1-methoxyethyl]-5-{2-oxa-6-azaspiro[3.4]octan-6-yl}pyridin-3-yl}-9-oxa-1-azatricyclo[6.3.1.0^{4,12}]dodeca-2,4(12),5,7-tetraen-3-yl)-2,2-dimethylpropan-1-ol. LC-MS (M+H)⁺=536.7.

### Step 3: methyl (4S)-2-[(2S)-2-{[(1R,2R,3S)-2,3-dimethylcyclopropyl]formamido}-3-{4-[3-(3-hydroxy-2,2-dimethylpropyl)-2-{2-[(1S)-1-methoxyethyl]-5-{2-oxa-6-azaspiro[3.4]octan-6-yl}pyridin-3-yl}-9-oxa-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylate

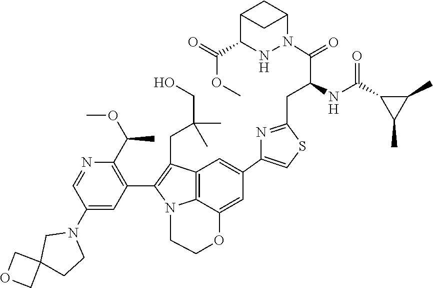 [SMILES: COC(=O)[C@H]1NN(C(=O)[C@H](Cc2nc(-c3cc4c5c(c3)c(CC(C)(C)CO)c(-c3cc(N6CCC7(COC7)C6)cnc3[C@H](C)OC)n5CCO4)cs2)NC(=O)[C@H]2[C@H](C)[C@@H]2C)C2CC1C2]

The title compound (140 mg, 62%) was prepared in a manner similar to that in example 47 step 5 from [3-(3-hydroxy-2,2-dimethylpropyl)-2-{2-[(1S)-1-methoxyethyl]-5-{2-oxa-6-azaspiro[3.4]octan-6-yl}pyridin-3-yl}-9-oxa-1-azatricyclo[6.3.1.0^{4,12}]dodeca-2,4(12),5,7-tetraen-6-yl]boronic acid and methyl (4S)-2-[(2S)-3-(4-bromo-1,3-thiazol-2-yl)-2-{[(1R,2R,3S)-2,3-dimethylcyclopropyl]formamido}propanoyl]-2,3-diazabicyclo[3.1.1]heptane-4-carboxylate. LC-MS (M+H)⁺=896.5.

### Step 4: (4S)-2-[(2S)-2-{[(1R,2R,3S)-2,3-dimethylcyclopropyl]formamido}-3-{4-[3-(3-hydroxy-2,2-dimethylpropyl)-2-{2-[(1S)-1-methoxyethyl]-5-{2-oxa-6-azaspiro[3.4]octan-6-yl}pyridin-3-yl}-9-oxa-1-azatricyclo[6.3.1.0⁴¹²]dodeca-2,4(12),5,7-tetraen-6-yl]-1,3-thiazol-2-yl}propanoyl]-2,3-diazabicyclo[3.1.1]heptane-4-carboxylic Acid

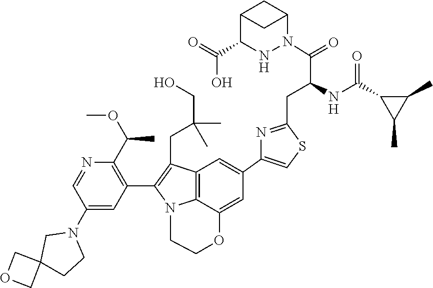 [SMILES: CO[C@@H](C)c1ncc(N2CCC3(COC3)C2)cc1-c1c(CC(C)(C)CO)c2cc(-c3csc(C[C@H](NC(=O)[C@H]4[C@H](C)[C@@H]4C)C(=O)N4N[C@H](C(=O)O)C5CC4C5)n3)cc3c2n1CCO3]

The title compound (120 mg, 87%) was prepared in a manner similar to that in example 47 step 6 from methyl (4S)-2-[(2S)-2-{[(1R,2R,3S)-2,3-dimethylcyclopropyl]formamido}-3-{4-[3-(3-hydroxy-2,2-dimethylpropyl)-2-{2-[(1S)-1-methoxyethyl]-5-{2-oxa-6-azaspiro[3.4]octan-6-yl}pyridin-3-yl}-9-oxa-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylate. LC-MS (M+H)⁺=882.3.

### Step 5: (1R,2R,3S)—N-[(7S,13S,19M)-20-{2-[(1S)-1-methoxyethyl]-5-{2-oxa-6-azaspiro[3.4]octan-6-yl}pyridin-3-yl}-17,17-dimethyl-8,14-dioxo-15,24-dioxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

To a solution of (4S)-2-[(2S)-2-{[(R,2R,3S)-2,3-dimethylcyclopropyl]formamido}-3-{4-[3-(3-hydroxy-2,2-dimethylpropyl)-2-{2-[(1S)-1-methoxyethyl]-5-{2-oxa-6-azaspiro[3.4]octan-6-yl}pyridin-3-yl}-9-oxa-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic acid (120 mg, 0.136 mmol) in anhydrous MeCN (50 mL) was added TCFH (115 mg, 0.408 mmol) and NMI (56 mg, 0.68 mmol). The mixture was stirred for 2 h at room temperature. Then the mixture was diluted with water (60 mL) and extracted with DCM (50 mL×5). The combined organic layer was washed with brine (30 mL×2), dried over Na₂SO₄, filtered and concentrated under vacuum. The residue was purified by prep-TLC (DCM:MeOH=10:1) and further purified by prep-HPLC to give example 48 (10 mg, 8%).

Example 48: ¹H NMR (400 MHz, DMSO-d6) δ 8.44 (d, J=8.9 Hz, 1H), 8.12-8.03 (m, 2H), 7.78 (s, 1H), 7.16 (s, 1H), 6.74 (d, J=2.4 Hz, 1H), 5.91 (d, J=11.0 Hz, 1H), 5.33 (t, J=8.1 Hz, 1H), 4.72 (d, J=11.0 Hz, 1H), 4.67-4.46 (m, 7H), 4.39-4.25 (m, 2H), 3.69 (t, J=9.3 Hz, 1H), 3.62-3.53 (m, 4H), 3.31-3.20 (m, 5H), 3.12 (dd, J=14.8, 7.3 Hz, 1H), 2.93 (d, J=14.3 Hz, 1H), 2.70-2.62 (m, 1H), 2.54 (d, J=2.1 Hz, 1H), 2.39-2.24 (m, 3H), 2.14 (t, J=9.8 Hz, 1H), 1.61 (t, J=9.3 Hz, 1H), 1.34 (d, J=6.0 Hz, 3H), 1.29-1.13 (m, 4H), 1.13-1.03 (m, 6H), 0.94 (s, 3H), 0.39 (s, 3H). LC-MS (M+H)⁺=864.65.

### Example 49: (1R,2R,3S)—N-[(7S,13S,19M)-20-{2-[(1S)-1-methoxyethyl]-5-{7-oxa-2-azaspiro[3.5]nonan-2-yl}pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

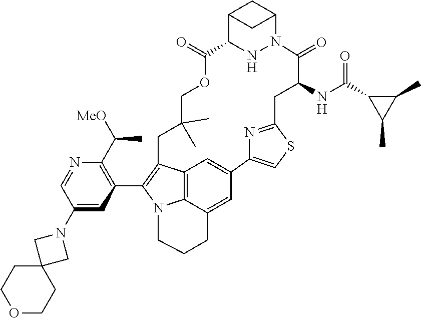

### Step 1: 3-(6-bromo-2-{2-[(1S)-1-methoxyethyl]-5-{7-oxa-2-azaspiro[3.5]nonan-2-yl}pyridin-3-yl}-1-azatricyclo[6.3.1.0^{4,12}]dodeca-2,4(12),5,7-tetraen-3-yl)-2,2-dimethylpropyl Acetate

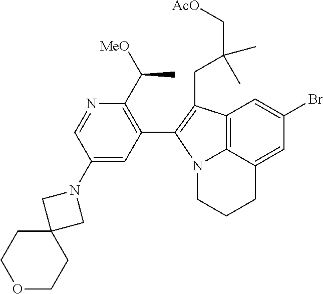 [SMILES: CO[C@@H](C)c1ncc(N2CC3(CCOCC3)C2)cc1-c1c(CC(C)(C)COC(C)=O)c2cc(Br)cc3c2n1CCC3]

The title compound (250 mg, 72%) was prepared in a manner similar to that in example 7 step 2 from 3-(6-bromo-2-{2-[(1S)-1-methoxyethyl]-5-(4,4,5,5-tetramethyl-1,3,2-dioxaborolan-2-yl)pyridin-3-yl}-1-azatricyclo[6.3.1.0^{4,12}]dodeca-2,4(12),5,7-tetraen-3-yl)-2,2-dimethylpropyl acetate and 7-oxa-2-azaspiro[3.5]nonane. LC-MS (M+H)⁺=624.3/626.3

### Step 2: {3-[3-(acetyloxy)-2,2-dimethylpropyl]-2-{2-[(1S)-1-methoxyethyl]-5-{7-oxa-2-azaspiro[3.5]nonan-2-yl}pyridin-3-yl}-1-azatricyclo[6.3.1.0^{4,12}]dodeca-2,4(12),5,7-tetraen-6-yl}boronic Acid

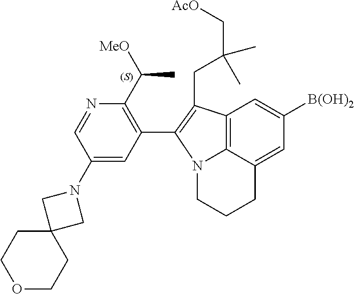

The title compound (131 mg, 67%) was prepared in a manner similar to that in example 32 step 7 from 3-(6-bromo-2-{2-[(1S)-1-methoxyethyl]-5-{7-oxa-2-azaspiro[3.5]nonan-2-yl}pyridin-3-yl}-1-azatricyclo[6.3.1.0^{4,12}]dodeca-2,4(12),5,7-tetraen-3-yl)-2,2-dimethylpropyl acetate. LC-MS (M+H)⁺=590.5.

### Step 3: methyl (4S)-2-[(2S)-3-(4-{3-[3-(acetyloxy)-2,2-dimethylpropyl]-2-{2-[(1S)-1-methoxyethyl]-5-{7-oxa-2-azaspiro[3.5]nonan-2-yl}pyridin-3-yl}-1-azatricyclo[6.3.1.0^{4,12}]dodeca-2,4(12),5,7-tetraen-6-yl}-1,3-thiazol-2-yl)-2-{[(tert-butoxy)carbonyl]amino}propanoyl]-2,3-diazabicyclo[3.1.1]heptane-4-carboxylate

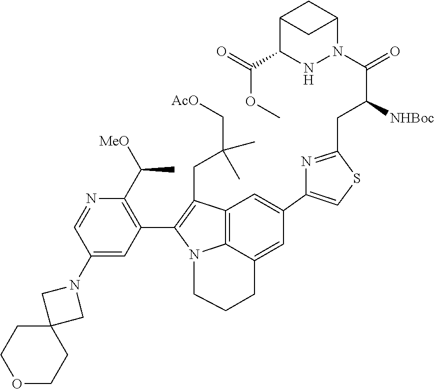 [SMILES: COC(=O)[C@H]1NN(C(=O)[C@H](Cc2nc(-c3cc4c5c(c3)c(CC(C)(C)COC(C)=O)c(-c3cc(N6CC7(CCOCC7)C6)cnc3[C@H](C)OC)n5CCC4)cs2)NC(=O)OC(C)(C)C)C2CC1C2]

The title compound (242 mg, 62%) was prepared in a manner similar to that in example 9 step 4 from {3-[3-(acetyloxy)-2,2-dimethylpropyl]-2-{2-[(1S)-1-methoxyethyl]-5-{7-oxa-2-azaspiro[3.5]nonan-2-yl}pyridin-3-yl}-1-azatricyclo[6.3.1.0^{4,12}]dodeca-2,4(12),5,7-tetraen-6-yl}boronic acid and methyl (4S)-2-[(2S)-3-(4-bromo-1,3-thiazol-2-yl)-2-{[(tert-butoxy)carbonyl]amino}propanoyl]-2,3-diazabicyclo[3.1.1]heptane-4-carboxylate. LC-MS (M+H)⁺=954.7.

### Step 4: (4S)-2-[(2S)-2-{[(tert-butoxy)carbonyl]amino}-3-{4-[3-(3-hydroxy-2,2-dimethylpropyl)-2-{2-[(1S)-1-methoxyethyl]-5-{7-oxa-2-azaspiro[3.5]nonan-2-yl}pyridin-3-yl}-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic Acid

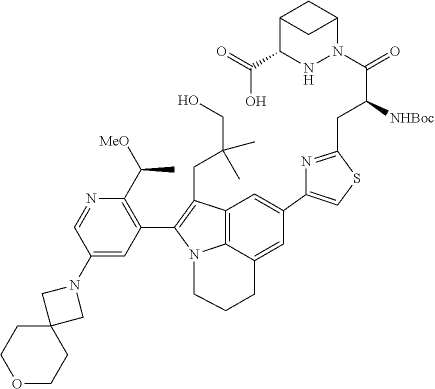 [SMILES: CO[C@@H](C)c1ncc(N2CC3(CCOCC3)C2)cc1-c1c(CC(C)(C)CO)c2cc(-c3csc(C[C@H](NC(=O)OC(C)(C)C)C(=O)N4N[C@H](C(=O)O)C5CC4C5)n3)cc3c2n1CCC3]

The title compound (210 mg, 93%) was prepared in a manner similar to that in example 9 step 5 form methyl (4S)-2-[(2S)-3-(4-{3-[3-(acetyloxy)-2,2-dimethylpropyl]-2-{2-[(1S)-1-methoxyethyl]-5-{7-oxa-2-azaspiro[3.5]nonan-2-yl}pyridin-3-yl}-1-azatricyclo[6.3.1.0^{4,12}]dodeca-2,4(12),5,7-tetraen-6-yl}-1,3-thiazol-2-yl)-2-{[(tert-butoxy)carbonyl]amino}propanoyl]-2,3-diazabicyclo[3.1.1]heptane-4-carboxylate. LC-MS (M+H)⁺=898.8.

### Step 5: tert-butyl N-[(7S,13S)-20-{2-[(1S)-1-methoxyethyl]-5-{7-oxa-2-azaspiro[3.5]nonan-2-yl}pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate

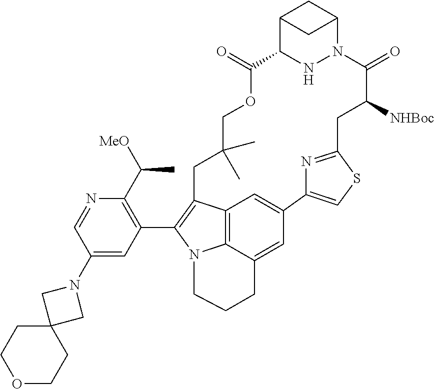 [SMILES: CO[C@@H](C)c1ncc(N2CC3(CCOCC3)C2)cc1-c1c2c3cc(cc4c3n1CCC4)-c1csc(n1)C[C@H](NC(=O)OC(C)(C)C)C(=O)N1N[C@H](C(=O)OCC(C)(C)C2)C2CC1C2]

The title compound (71 mg, 34%) was prepared in a manner similar to that in example 9 step 6 form (4S)-2-[(2S)-2-{[(tert-butoxy)carbonyl]amino)-3-{4-[3-(3-hydroxy-2,2-dimethylpropyl)-2-{2-[(1S)-1-methoxyethyl]-5-(7-oxa-2-azaspiro[3.5]nonan-2-yl}pyridin-3-yl}-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic acid. LC-MS (M+H)⁺=880.8.

### Step 6: (7S,13S)-7-amino-20-{2-[(1S)-1-methoxyethyl]-5-{7-oxa-2-azaspiro[3.5]nonan-2-yl}pyridin-3-yl}-17,17-dimethyl-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione

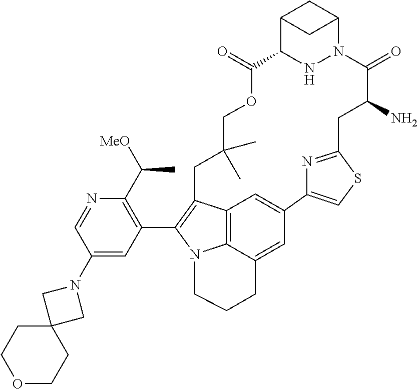 [SMILES: CO[C@@H](C)c1ncc(N2CC3(CCOCC3)C2)cc1-c1c2c3cc(cc4c3n1CCC4)-c1csc(n1)C[C@H](N)C(=O)N1N[C@H](C(=O)OCC(C)(C)C2)C2CC1C2]

The title compound (60 mg, 43%) was prepared in a manner similar to that in example 9 step 7 from tert-butyl N-[(7S,13S)-20-{2-[(1S)-1-methoxyethyl]-5-{7-oxa-2-azaspiro[3.5]nonan-2-yl}pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate. LC-MS (M+H)⁺=780.8.

### Step 7: (1R,2R,3S)—N-[(7S,13S,19M)-20-{2-[(1S)-1-methoxyethyl]-5-{7-oxa-2-azaspiro[3.5]nonan-2-yl}pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

Example 49 (17.5 mg, 25%) was prepared in a manner similar to that in example 9 step 8 from (7S,13S)-7-amino-20-{2-[(1S)-1-methoxyethyl]-5-{7-oxa-2-azaspiro[3.5]nonan-2-yl}pyridin-3-yl}-17,17-dimethyl-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione and (1r,2R,3S)-2,3-dimethylcyclopropane-1-carboxylic acid. ¹H NMR (400 MHz, DMSO-d6) δ 8.37 (d, J=8.4, 1H), 8.19 (s, 1H), 7.90 (s, 1H), 7.67 (s, 1H), 7.39 (s, 1H), 6.58 (s, 1H), 5.88 (d, J=11.0, 1H), 5.31 (s, 1H), 4.64 (d, J=11.2, 1H), 4.44 (s, 1H), 4.27-4.11 (m, 2H), 3.73-3.38 (m, 11H), 3.24-3.03 (m, 5H), 3.01-2.78 (m, 3H), 2.62 (s, 1H), 2.43-2.35 (m, 2H), 2.32-2.02 (m, 4H), 1.71 (s, 4H), 1.57-1.47 (m, 1H), 1.27 (d, J=5.4, 3H), 1.23-0.95 (m, 9H), 0.85 (s, 3H), 0.34 (s, 3H). LC-MS (M+H)⁺=876.3.

### Example 50: (1R,2R,3S)—N-[(7S,13S,19M)-20-{5-[(3R,5S)-4-ethyl-3,5-dimethylpiperazin-1-yl]-2-[(1S)-1-methoxyethyl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

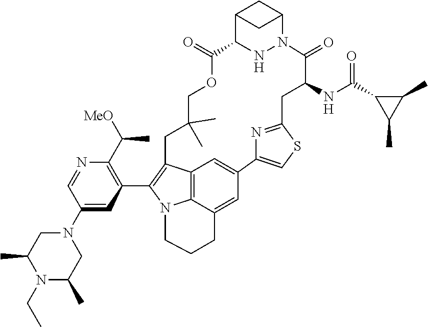

### Step 1: 3-[(2M)-6-bromo-2-{2-[(1S)-1-methoxyethyl]pyridin-3-yl}-1-azatricyclo[6.3.1.0^{4,12}]dodeca-2,4(12),5,7-tetraen-3-yl]-2,2-dimethylpropan-1-ol

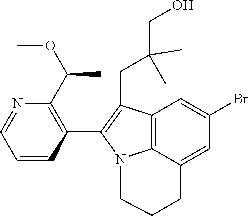

The atropisomers of 3-(6-bromo-2-{2-[(1S)-1-methoxyethyl]pyridin-3-yl}-1-azatricyclo[6.3.1.0^{4,12}]dodeca-2,4(12),5,7-tetraen-3-yl)-2,2-dimethylpropan-1-ol (60 g) were purified by silica gel column chromatography (PE:EA=1:1 to PE:EA=1:7) to give (M-isomer, 30 g, 50%) and (P-isomer, 25 g, 42%). P-isomer: ¹H NMR (400 MHz, DMSO-d6) δ 8.70 (dd, J=4.8, 1.8 Hz, 1H), 7.77 (dd, J=7.8, 1.8 Hz, 1H), 7.65 (d, J=1.7 Hz, 1H), 7.43 (dd, J=7.7, 4.7 Hz, 1H), 7.21-6.88 (m, 1H), 4.48 (t, J=5.2 Hz, 1H), 4.05 (q, J=6.3 Hz, 1H), 3.90-3.65 (m, 1H), 3.53-3.36 (m, 1H), 3.14-3.02 (m, 4H), 2.97-2.85 (m, 3H), 2.67 (d, J=14.0 Hz, 1H), 2.23 (d, J=14.1 Hz, 1H), 2.14-2.04 (m, 1H), 2.00-1.87 (m, 1H), 1.10 (d, J=6.3 Hz, 3H), 0.60 (s, 6H). LC-MS (M+H)⁺=457.3/459.3. M-isomer: ¹H NMR (400 MHz, DMSO-d6) δ 8.70 (dd, J=4.7, 1.7 Hz, 1H), 7.79 (dd, J=7.8, 1.7 Hz, 1H), 7.62 (d, J=1.8 Hz, 1H), 7.45 (dd, J=7.8, 4.7 Hz, 1H), 7.10-6.93 (m, 1H), 4.46 (t, J=5.2 Hz, 1H), 4.13 (q, J=6.2 Hz, 1H), 3.90-3.72 (m, 1H), 3.66-3.51 (m, 1H), 3.03 (dd, J=10.3, 5.4 Hz, 1H), 2.94-2.82 (m, 3H), 2.72 (s, 3H), 2.63 (d, J=13.9 Hz, 1H), 2.22-2.02 (m, 2H), 2.01-1.84 (m, 1H), 1.38 (d, J=6.2 Hz, 3H), 0.56 (s, 3H), 0.52 (s, 3H). LC-MS (M+H)⁺=457.3/459.3.

### Step 2: 3-[(2M)-6-bromo-2-{2-[(1S)-1-methoxyethyl]-5-(4,4,5,5-tetramethyl-1,3,2-dioxaborolan-2-yl)pyridin-3-yl}-1-azatricyclo[6.3.1.0^{4,12}]dodeca-2,4(12),5,7-tetraen-3-yl]-2,2-dimethylpropan-1-ol

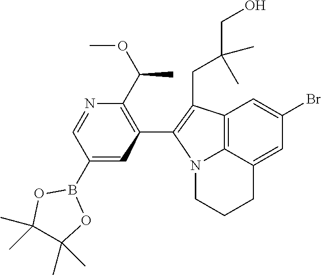

To a mixture of 3-[(2M)-6-bromo-2-{2-[(1S)-1-methoxyethyl]pyridin-3-yl}-1-azatricyclo[6.3.1.0^{4,12}]dodeca-2,4(12),5,7-tetraen-3-yl]-2,2-dimethylpropan-1-ol (78 g, 170 mmol) in n-heptane (300 mL) and THF (150 mL) was added pinacolborane (43 g, 336 mmol) dropwise at 0° C. The resulting mixture was heated to 50° C. and stirred for 5 h at 50° C. The reaction mixture was then cooled to 25° C., followed by the addition of B₂Pin₂ (60 g, 236 mmol), 3,4,7,8-tetramethyl-1,10-phenanthroline (0.75 g, 3.2 mmol) and (1,5-cyclooctadiene)(methoxy)iridium(I) dimer (0.52 g, 0.78 mmol). The resulting mixture was heated to 50° C. and stirred for 15 h. The reaction mixture was poured into ice-water (1 L) and extracted with EA (1 L×2). The combined organic layers were washed with brine (1 L), dried over Na₂SO₄, filtered and concentrated under vacuum to give the title compound (90 g, 90%). LC-MS (M-pinacol+2H₂O+H)⁺=501.3/503.3.

### Step 3: (2S,6R)-1-ethyl-2,6-dimethylpiperazine Trifluoroacetate

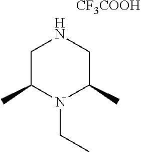

To a solution of tert-butyl (3S,5R)-3,5-dimethylpiperazine-1-carboxylate (5.0 g, 23.4 mmol) in DMF (40 mL) was added iodoethane (10.9 g, 70.1 mmol) and K₂CO₃ (6.4 g, 46.7 mmol). The reaction mixture was heated to 80° C. and stirred overnight. The mixture was cooled to room temperature and diluted with water (200 mL). The mixture was extracted with EA (100 mL) and washed with brine (100 mL). The organic layer was dried over Na₂SO₄, filtered and concentrated in vacuo to give a residue. The residue was dissolved in DCM (50 mL), and trifluoroacetic acid (25 mL) was added. The mixture was stirred at rt for 2 h. Volatiles were removed in vacuo to give the title compound, which was used in the next step without further purification. LC-MS (M+H)⁺=143.1.

### Step 4: 3-[(2M)-6-bromo-2-{5-[(3R,5S)-4-ethyl-3,5-dimethylpiperazin-1-yl]-2-[(1S)-1-methoxyethyl]pyridin-3-yl}-1-azatricyclo[6.3.1.0^{4,12}]dodeca-2,4(12),5,7-tetraen-3-yl]-2,2-dimethylpropan-1-ol

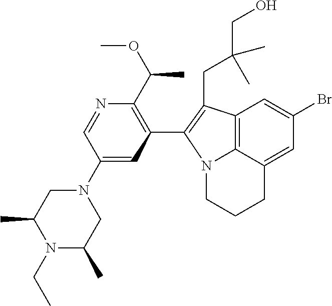

The title compound (158 mg, 50%) was prepared in a manner similar to that in example 9 step 2 from 3-[(2M)-6-bromo-2-{2-[(1S)-1-methoxyethyl]-5-(4,4,5,5-tetramethyl-1,3,2-dioxaborolan-2-yl)pyridin-3-yl}-1-azatricyclo[6.3.1.0^{4,12}]dodeca-2,4(12),5,7-tetraen-3-yl]-2,2-dimethylpropan-1-ol and (2S,6R)-1-ethyl-2,6-dimethylpiperazine trifluoroacetate. LC-MS (M+H)⁺=597.2/599.2.

### Step 5: [(2M)-2-{5-[(3R,5S)-4-ethyl-3,5-dimethylpiperazin-1-yl]-2-[(1S)-1-methoxyethyl]pyridin-3-yl}-3-(3-hydroxy-2,2-dimethylpropyl)-1-azatricyclo[6.3.1.0^{4,12}]dodeca-2,4(12),5,7-tetraen-6-yl]boronic Acid

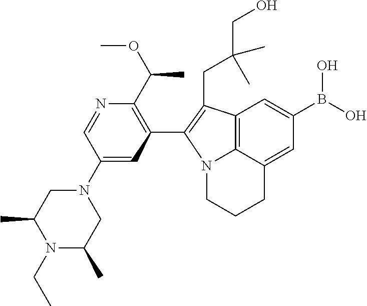

The title compound (258 mg, 46%) was prepared in a manner similar to that in example 32 step 7 from 3-[(2M)-6-bromo-2-{5-[(3R,5S)-4-ethyl-3,5-dimethylpiperazin-1-yl]-2-[(1S)-1-methoxyethyl]pyridin-3-yl}-1-azatricyclo[6.3.1.0^{4,12}]dodeca-2,4(12),5,7-tetraen-3-yl]-2,2-dimethylpropan-1-ol. LC-MS (M+H)⁺=563.3.

### Step 6: (4S)-2-[(2S)-2-{[(1R,2R,3S)-2,3-dimethylcyclopropyl]formamido}-3-{4-[(2M)-2-{5-[(3R,5S)-4-ethyl-3,5-dimethylpiperazin-1-yl]-2-[(1S)-1-methoxyethyl]pyridin-3-yl}-3-(3-hydroxy-2,2-dimethylpropyl)-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic Acid

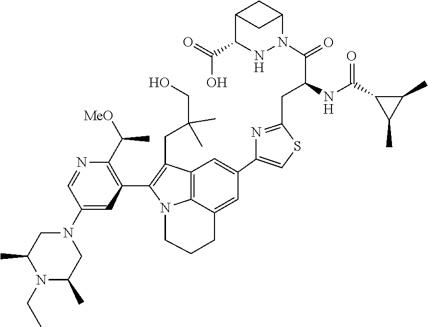

The title compound (50 mg, 30%) was prepared in a manner similar to that in example 32 step 8 from [(2M)-2-{5-[(3R,5S)-4-ethyl-3,5-dimethylpiperazin-1-yl]-2-[(1S)-1-methoxyethyl]pyridin-3-yl}-3-(3-hydroxy-2,2-dimethylpropyl)-1-azatricyclo[6.3.1.0^{4,12}]dodeca-2,4(12),5,7-tetraen-6-yl]boronic acid and (4S)-2-[(2S)-3-(4-bromo-1,3-thiazol-2-yl)-2-{[(R,2R,3S)-2,3-dimethylcyclopropyl]formamido}propanoyl]-2,3-diazabicyclo[3.1.1]heptane-4-carboxylic acid. LC-MS (M+H)⁺=909.5.

### Step 7: (1R,2R,3S)—N-[(7S,13S,19M)-20-{5-[(3R,5S)-4-ethyl-3,5-dimethylpiperazin-1-yl]-2-[(1S)-1-methoxyethyl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

Example 50 (50 mg, 30%) was prepared in a manner similar to that in example 32 step 9 from (4S)-2-[(2S)-2-{[(1R,2R,3S)-2,3-dimethylcyclopropyl]formamido}-3-{4-[(2M)-2-{5-[(3R,5S)-4-ethyl-3,5-dimethylpiperazin-1-yl]-2-[(1S)-1-methoxyethyl]pyridin-3-yl}-3-(3-hydroxy-2,2-dimethylpropyl)-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic acid. ¹H NMR (400 MHz, DMSO-d6) δ 8.47-8.29 (m, 2H), 8.21 (s, 1H), 7.69 (s, 1H), 7.40 (s, 1H), 7.19-7.06 (m, 1H), 5.90 (d, J=11.1 Hz, 1H), 5.50-5.24 (m, 1H), 4.64 (d, J=10.9 Hz, 1H), 4.52-4.42 (m, 1H), 4.33-4.07 (m, 2H), 3.68 (d, J=11.6 Hz, 2H), 3.62-3.41 (m, 3H), 3.26-3.14 (m, 4H), 3.09 (dd, J=14.9, 7.1 Hz, 1H), 3.01-2.95 (m, 1H), 2.90-2.72 (m, 4H), 2.68-1.99 (m, 11H), 1.65-1.46 (m, 1H), 1.30 (d, J=6.0 Hz, 3H), 1.26-1.09 (m, 3H), 1.08-0.96 (m, 12H), 0.85 (s, 3H), 0.81 (t, J=7.0 Hz, 3H), 0.36 (s, 3H). LC-MS (M+H)⁺=891.4.

### Example 51: (1R,2R,3S)—N-[(7S,13S,19M)-20-{2-[(1S)-1-methoxyethyl]-5-[(3S,5S)-3,4,5-trimethylpiperazin-1-yl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

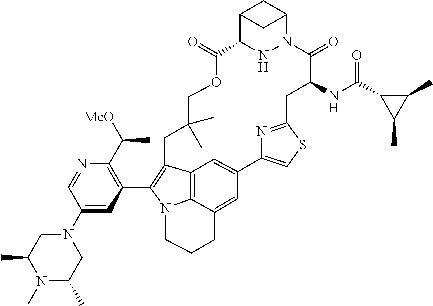

### Step 1: tert-butyl (2S,6S)-4-[(5M)-5-[6-bromo-3-(3-hydroxy-2,2-dimethylpropyl)-1-azatricyclo[6.3.1.0^{4,12}]dodeca-2,4(12),5,7-tetraen-2-yl]-6-[(1S)-1-methoxyethyl]pyridin-3-yl]-2,6-dimethylpiperazine-1-carboxylate

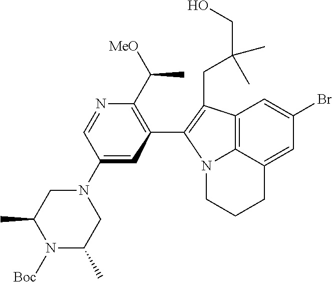

The title compound (0.7 g, 41%) was prepared in a manner similar to that in example 20 step 1 from 3-[(2M)-6-bromo-2-{2-[(1S)-1-methoxyethyl]-5-(4,4,5,5-tetramethyl-1,3,2-dioxaborolan-2-yl)pyridin-3-yl}-1-azatricyclo[6.3.1.0^{4,12}]dodeca-2,4(12),5,7-tetraen-3-yl]-2,2-dimethylpropan-1-ol and tert-butyl (2S,6S)-2,6-dimethylpiperazine-1-carboxylate. LC-MS (M+H)⁺=669.3/671.3.

### Step 2: 3-[(2M)-6-bromo-2-{5-[(3S,5S)-3,5-dimethylpiperazin-1-yl]-2-[(1S)-1-methoxyethyl]pyridin-3-yl}-1-azatricyclo[6.3.1.0^{4,12}]dodeca-2,4(12),5,7-tetraen-3-yl]-2,2-dimethylpropan-1-ol

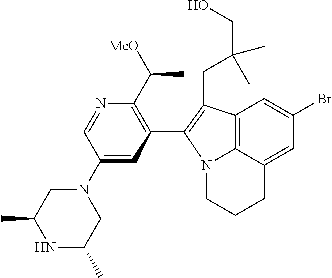

The title compound (0.5 g, 84%) was prepared in a manner similar to that in example 26 step 2 from tert-butyl (2S,6S)-4-[(5M)-5-[6-bromo-3-(3-hydroxy-2,2-dimethylpropyl)-1-azatricyclo[6.3.1.0^{4,12}]dodeca-2,4(12),5,7-tetraen-2-yl]-6-[(1S)-1-methoxyethyl]pyridin-3-yl]-2,6-dimethylpiperazine-1-carboxylate. LC-MS (M+H)⁺=569.3/571.3.

### Step 3: 3-[(2M)-6-bromo-2-{2-[(1S)-1-methoxyethyl]-5-[(3S,5S)-3,4,5-trimethylpiperazin-1-yl]pyridin-3-yl}-1-azatricyclo[6.3.1.0^{4,12}]dodeca-2,4(12),5,7-tetraen-3-yl]-2,2-dimethylpropan-1-ol

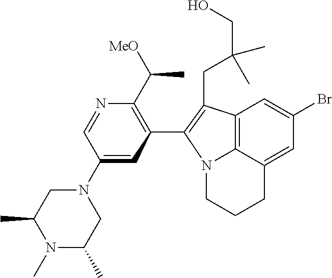

To a mixture of 3-[(2M)-6-bromo-2-{5-[(3S,5S)-3,5-dimethylpiperazin-1-yl]-2-[(1S)-1-methoxyethyl]pyridin-3-yl}-1-azatricyclo[6.3.1.0^{4,12}]dodeca-2,4(12),5,7-tetraen-3-yl]-2,2-dimethylpropan-1-ol (0.5 g, 0.88 mmol) in DCM (10 mL) was added formaldehyde (0.28 g, 4.4 mmol, 37% in water) and sodium triacetoxyborohydride (373 mg, 1.76 mmol). The mixture was stirred for 3 h at room temperature. The reaction was diluted with DCM (10 mL), washed with saturated NaHCO₃ (10 mL), dried over Na₂SO₄, filtered, and concentrated under vacuum. The residue was purified by silica gel column chromatography (DCM:MeOH=15:1) to give the title compound (0.5 g, 97%). LC-MS (M+H)⁺=583.3/585.3.

### Step 4: [(2M)-3-(3-hydroxy-2,2-dimethylpropyl)-2-{2-[(1S)-1-methoxyethyl]-5-[(3S,5S)-3,4,5-trimethylpiperazin-1-yl]pyridin-3-yl}-1-azatricyclo[6.3.1.0^{4,12}]dodeca-2,4(12),5,7-tetraen-6-yl]boronic Acid

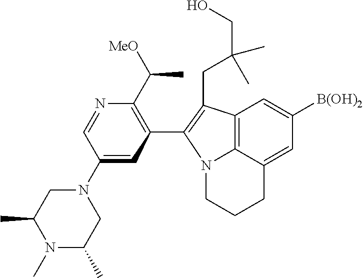

The title compound (0.4 g, 85%) was prepared in a manner similar to that in example 35 step 3 from 3-[(2M)-6-bromo-2-{2-[(1S)-1-methoxyethyl]-5-[(3S,5S)-3,4,5-trimethylpiperazin-1-yl]pyridin-3-yl}-1-azatricyclo[6.3.1.0^{4,12}]dodeca-2,4(12),5,7-tetraen-3-yl]-2,2-dimethylpropan-1-ol and B₂(OH)₄. LC-MS (M+H)⁺=549.3

### Step 5: methyl (4S)-2-[(2S)-2-{[(1R,2R,3S)-2,3-dimethylcyclopropyl]formamido}-3-{4-[(2M)-3-(3-hydroxy-2,2-dimethylpropyl)-2-{2-[(1S)-1-methoxyethyl]-5-[(3S,5S)-3,4,5-trimethylpiperazin-1-yl]pyridin-3-yl}-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylate

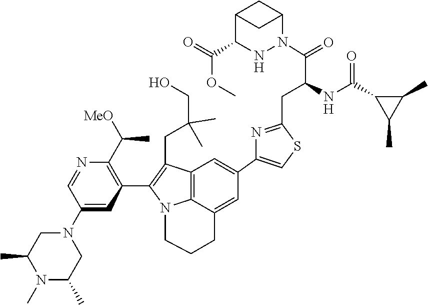

The title compound (0.3 g, 77%) was prepared in a manner similar to that in example 35 step 4 from [(2M)-3-(3-hydroxy-2,2-dimethylpropyl)-2-{2-[(1S)-1-methoxyethyl]-5-[(3S,5S)-3,4,5-trimethylpiperazin-1-yl]pyridin-3-yl}-1-azatricyclo[6.3.1.0^{4,12}]dodeca-2,4(12),5,7-tetraen-6-yl]boronic acid and methyl (4S)-2-[(2S)-3-(4-bromo-1,3-thiazol-2-yl)-2-{[(1R,2R,3S)-2,3-dimethylcyclopropyl]formamido}propanoyl]-2,3-diazabicyclo[3.1.1]heptane-4-carboxylate. LC-MS (M+H)⁺=909.5

### Step 6: (4S)-2-[(2S)-2-{[(1R,2R,3S)-2,3-dimethylcyclopropyl]formamido}-3-{4-[(2M)-3-(3-hydroxy-2,2-dimethylpropyl)-2-{2-[(1S)-1-methoxyethyl]-5-[(3S,5S)-3,4,5-trimethylpiperazin-1-yl]pyridin-3-yl}-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic Acid

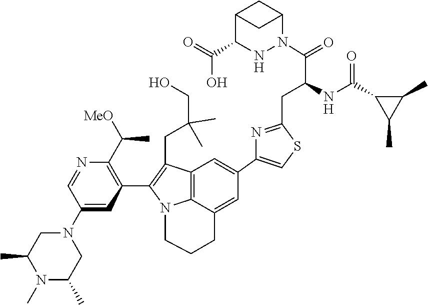

The title compound (0.25 g, 90%) was prepared in a manner similar to that in example 35 step 5 from methyl (4S)-2-[(2S)-2-{[(1R,2R,3S)-2,3-dimethylcyclopropyl]formamido}-3-{4-[(2M)-3-(3-hydroxy-2,2-dimethylpropyl)-2-{2-[(1S)-1-methoxyethyl]-5-[(3S,5S)-3,4,5-trimethylpiperazin-1-yl]pyridin-3-yl}-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylate. LC-MS (M+H)⁺=895.5

### Step 7: (1R,2R,3S)—N-[(7S,13S,19M)-20-{2-[(1S)-1-methoxyethyl]-5-[(3S,5S)-3,4,5-trimethylpiperazin-1-yl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

Example 51 (35 mg, 30%) was prepared in a manner similar to that in example 35 step 6 from (4S)-2-[(2S)-2-{[(R,2R,3S)-2,3-dimethylcyclopropyl]formamido}-3-{4-[(2M)-3-(3-hydroxy-2,2-dimethylpropyl)-2-{2-[(1S)-1-methoxyethyl]-5-[(3S,5S)-3,4,5-trimethylpiperazin-1-yl]pyridin-3-yl}-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic acid.

¹H NMR (400 MHz, DMSO-d6) δ 8.46-8.30 (m, 2H), 8.21 (s, 1H), 7.69 (s, 1H), 7.40 (s, 1H), 7.11 (s, 1H), 5.90 (d, J=11.0 Hz, 1H), 5.56-5.14 (m, 1H), 4.64 (d, J=11.0 Hz, 1H), 4.52-4.38 (m, 1H), 4.35-4.03 (m, 2H), 3.78-3.37 (m, 3H), 3.26-3.14 (m, 6H), 3.09 (dd, J=14.8, 7.3 Hz, 1H), 3.00-2.78 (m, 7H), 2.64-2.56 (m, 1H), 2.53-2.03 (m, 9H), 1.52 (t, J=9.0 Hz, 1H), 1.30 (d, J=6.0 Hz, 3H), 1.27-1.10 (m, 3H), 1.08-0.94 (m, 12H), 0.86 (s, 3H), 0.36 (s, 3H). LC-MS (M+H)⁺=877.7.

### Examples 52: (1S,2S)—N-[(7S,13S,19M)-20-{2-[(1S)-1-methoxyethyl]-5-[(3S,5S)-3,4,5-trimethylpiperazin-1-yl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2-methylcyclopropane-1-carboxamide

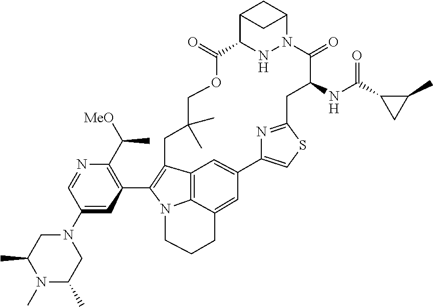

### Step 1: methyl (4S)-2-[(2S)-3-(4-bromo-1,3-thiazol-2-yl)-2-{[(1S,2S)-2-methylcyclopropyl]formamido}propanoyl]-2,3-diazabicyclo[3.1.1]heptane-4-carboxylate

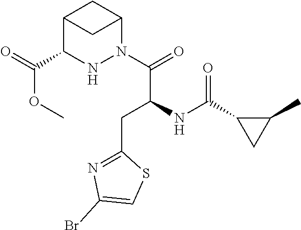 [SMILES: COC(=O)[C@H]1NN(C(=O)[C@H](Cc2nc(Br)cs2)NC(=O)[C@H]2C[C@@H]2C)C2CC1C2]

The title compound (30 g, 82%) was prepared in a manner similar to that in example 32 step 2 from methyl (4S)-2-[(2S)-2-amino-3-(4-bromo-1,3-thiazol-2-yl)propanoyl]-2,3-diazabicyclo[3.1.1]heptane-4-carboxylate hydrochloride and (1S,2S)-2-methylcyclopropane-1-carboxylic acid. LC-MS (M+H)⁺=471.3/473.3

### Step 2: methyl (4S)-2-[(2S)-3-{4-[(2M)-3-(3-hydroxy-2,2-dimethylpropyl)-2-{2-[(1S)-1-methoxyethyl]-5-[(3S,5S)-3,4,5-trimethylpiperazin-1-yl]pyridin-3-yl}-1-azatricyclo[6.3.1.0^{4,12}]dodeca-2,4(12),5,7-tetraen-6-yl]-1,3-thiazol-2-yl}-2-{[(1S,2S)-2-methylcyclopropyl]formamido}propanoyl]-2,3-diazabicyclo[3.1.1]heptane-4-carboxylate

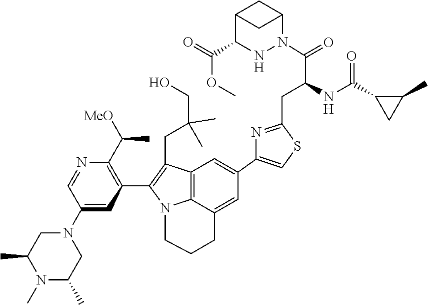

The title compound (0.28 g, 76%) was prepared in a manner similar to that in example 35 step 4 from [(2M)-3-(3-hydroxy-2,2-dimethylpropyl)-2-{2-[(1S)-1-methoxyethyl]-5-[(3S,5S)-3,4,5-trimethylpiperazin-1-yl]pyridin-3-yl}-1-azatricyclo[6.3.1.0^{4,12}]dodeca-2,4(12),5,7-tetraen-6-yl]boronic acid and methyl (4S)-2-[(2S)-3-(4-bromo-1,3-thiazol-2-yl)-2-{[(1S,2S)-2-methylcyclopropyl]formamido}propanoyl]-2,3-diazabicyclo[3.1.1]heptane-4-carboxylate. LC-MS (M+H)⁺=895.5

### Step 3: (4S)-2-[(2S)-3-{4-[(2M)-3-(3-hydroxy-2,2-dimethylpropyl)-2-{2-[(1S)-1-methoxyethyl]-5-[(3S,5S)-3,4,5-trimethylpiperazin-1-yl]pyridin-3-yl}-1-azatricyclo[6.3.1.0^{4,12}]dodeca-2,4(12),5,7-tetraen-6-yl]-1,3-thiazol-2-yl}-2-{[(1S,2S)-2-methylcyclopropyl]formamido}propanoyl]-2,3-diazabicyclo[3.1.1]heptane-4-carboxylic Acid

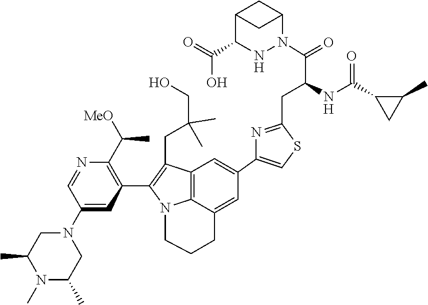

The title compound (0.2 g, 92%) was prepared in a manner similar to that in example 35 step 5 from methyl (4S)-2-[(2S)-3-{4-[(2M)-3-(3-hydroxy-2,2-dimethylpropyl)-2-{2-[(1S)-1-methoxyethyl]-5-[(3S,5S)-3,4,5-trimethylpiperazin-1-yl]pyridin-3-yl}-1-azatricyclo[6.3.1.0^{4,12}]dodeca-2,4(12),5,7-tetraen-6-yl]-1,3-thiazol-2-yl}-2-{[(S,2S)-2-methylcyclopropyl]formamido}propanoyl]-2,3-diazabicyclo[3.1.1]heptane-4-carboxylate. LC-MS (M+H)⁺=881.5

### Step 4: (1S,2S)—N-[(7S,13S,19M)-20-{2-[(1S)-1-methoxyethyl]-5-[(3S,5S)-3,4,5-trimethylpiperazin-1-yl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2-methylcyclopropane-1-carboxamide

Example 52 (42 mg, 37%) was prepared in a manner similar to that in example 35 step 6 from (4S)-2-[(2S)-3-{4-[(2M)-3-(3-hydroxy-2,2-dimethylpropyl)-2-(2-[(1S)-1-methoxyethyl]-5-[(3S,5S)-3,4,5-trimethylpiperazin-1-yl]pyridin-3-yl}-1-azatricyclo[6.3.1.0^{4,12}]dodeca-2,4(12),5,7-tetraen-6-yl]-1,3-thiazol-2-yl)-2-{[(1S,2S)-2-methylcyclopropyl]formamido}propanoyl]-2,3-diazabicyclo[3.1.1]heptane-4-carboxylic acid. ¹H NMR (400 MHz, DMSO-d6) δ 8.50 (d, J=8.9 Hz, 1H), 8.39 (d, J=2.8 Hz, 1H), 8.21 (s, 1H), 7.68 (s, 1H), 7.40 (s, 1H), 7.10 (d, J=2.9 Hz, 1H), 5.91 (d, J=11.0 Hz, 1H), 5.36 (t, J=8.1 Hz, 1H), 4.65 (d, J=11.0 Hz, 1H), 4.51-4.39 (m, 1H), 4.27-4.10 (m, 2H), 3.68-3.41 (m, 3H), 3.27-3.14 (m, 6H), 3.09 (dd, J=14.7, 7.4 Hz, 1H), 3.02-2.76 (m, 7H), 2.66-2.56 (m, 1H), 2.54-2.01 (m, 9H), 1.53 (t, J=9.3 Hz, 1H), 1.48-1.42 (m, 1H), 1.30 (d, J=6.0 Hz, 3H), 1.10-0.78 (m, 14H), 0.62-0.47 (m, 1H), 0.36 (s, 3H). LC-MS (M+H)⁺=863.4.

### Example 53: (1R,2R,3S)—N-[(7S,13S,19M)-20-{2-[(1S)-1-methoxyethyl]-5-[(3R,5R)-3,4,5-trimethylpiperazin-1-yl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

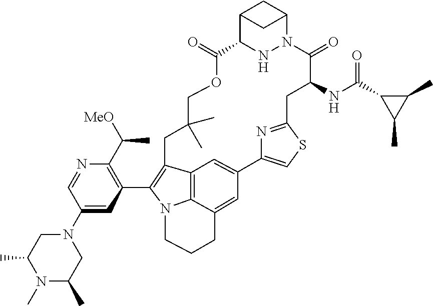

### Step 1: tert-butyl (2R,6R)-4-{5-[6-bromo-3-(3-hydroxy-2,2-dimethylpropyl)-1-azatricyclo[6.3.1.0^{4,12}]dodeca-2,4(12),5,7-tetraen-2-yl]-6-[(1S)-1-methoxyethyl]pyridin-3-yl}-2,6-dimethylpiperazine-1-carboxylate

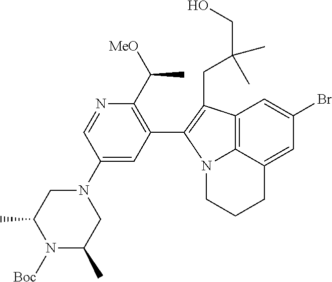 [SMILES: CO[C@@H](C)c1ncc(N2C[C@@H](C)N(C(=O)OC(C)(C)C)[C@H](C)C2)cc1-c1c(CC(C)(C)CO)c2cc(Br)cc3c2n1CCC3]

The title compound (804 mg, 24%) was prepared in a manner similar to that in example 20 step 1 from 3-[(2M)-6-bromo-2-{2-[(1S)-1-methoxyethyl]-5-(4,4,5,5-tetramethyl-1,3,2-dioxaborolan-2-yl)pyridin-3-yl}-1-azatricyclo[6.3.1.0^{4,12}]dodeca-2,4(12),5,7-tetraen-3-yl]-2,2-dimethylpropan-1-ol and tert-butyl (2R,6R)-2,6-dimethylpiperazine-1-carboxylate. LC-MS (M+H)⁺=669.4/671.4.

### Step 2: 3-(6-bromo-2-{5-[(3R,5R)-3,5-dimethylpiperazin-1-yl]-2-[(1S)-1-methoxyethyl]pyridin-3-yl}-1-azatricyclo[6.3.1.0^{4,12}]dodeca-2,4(12),5,7-tetraen-3-yl)-2,2-dimethylpropan-1-ol

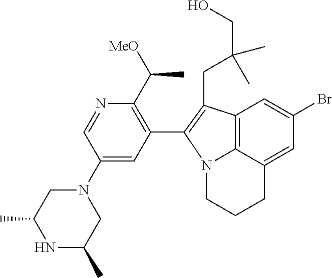 [SMILES: CO[C@@H](C)c1ncc(N2C[C@@H](C)N[C@H](C)C2)cc1-c1c(CC(C)(C)CO)c2cc(Br)cc3c2n1CCC3]

The title compound (683 mg, 99%) was prepared in a manner similar to that in example 26 step 2 from tert-butyl (2R,6R)-4-{5-[6-bromo-3-(3-hydroxy-2,2-dimethylpropyl)-1-azatricyclo[6.3.1.0^{4,12}]dodeca-2,4(12),5,7-tetraen-2-yl]-6-[(1S)-1-methoxyethyl]pyridin-3-yl}-2,6-dimethylpiperazine-1-carboxylate. LC-MS (M+H)⁺=569.4/571.4.

### Step 3: 3-(6-bromo-2-{2-[(1S)-1-methoxyethyl]-5-[(3R,5R)-3,4,5-trimethylpiperazin-1-yl]pyridin-3-yl}-1-azatricyclo[6.3.1.0^{4,12}]dodeca-2,4(12),5,7-tetraen-3-yl)-2,2-dimethylpropan-1-ol

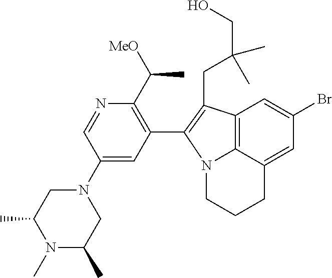 [SMILES: CO[C@@H](C)c1ncc(N2C[C@@H](C)N(C)[C@H](C)C2)cc1-c1c(CC(C)(C)CO)c2cc(Br)cc3c2n1CCC3]

The title compound (630 mg, 90%) was prepared in a manner similar to that in example 51 step 3 from 3-(6-bromo-2-{5-[(3R,5R)-3,5-dimethylpiperazin-1-yl]-2-[(1S)-1-methoxyethyl]pyridin-3-yl}-1-azatricyclo[6.3.1.0^{4,12}]dodeca-2,4(12),5,7-tetraen-3-yl)-2,2-dimethylpropan-1-ol. LC-MS (M+H)⁺=583.6/585.5

### Step 4: [3-(3-hydroxy-2,2-dimethylpropyl)-2-{2-[(1S)-1-methoxyethyl]-5-[(3R,5R)-3,4,5-trimethylpiperazin-1-yl]pyridin-3-yl}-1-azatricyclo[6.3.1.0^{4,12}]dodeca-2,4(12),5,7-tetraen-6-yl]boronic Acid

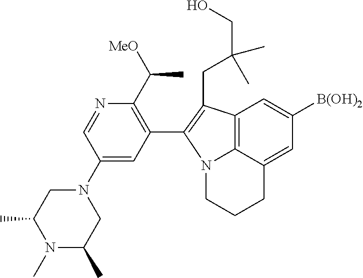 [SMILES: CO[C@@H](C)c1ncc(N2C[C@@H](C)N(C)[C@H](C)C2)cc1-c1c(CC(C)(C)CO)c2cc(B(O)O)cc3c2n1CCC3]

The title compound (573 mg, 97%) was prepared in a manner similar to that in example 35 step 3 from 3-(6-bromo-2-{2-[(1S)-1-methoxyethyl]-5-[(3R,5R)-3,4,5-trimethylpiperazin-1-yl]pyridin-3-yl}-1-azatricyclo[6.3.1.0^{4,12}]dodeca-2,4(12),5,7-tetraen-3-yl)-2,2-dimethylpropan-1-ol. LC-MS (M+H)⁺=549.6.

### Step 5: methyl (4S)-2-[(2S)-2-{[(1R,2R,3S)-2,3-dimethylcyclopropyl]formamido}-3-{4-[3-(3-hydroxy-2,2-dimethylpropyl)-2-{2-[(1S)-1-methoxyethyl]-5-[(3R,5R)-3,4,5-trimethylpiperazin-1-yl]pyridin-3-yl}-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylate

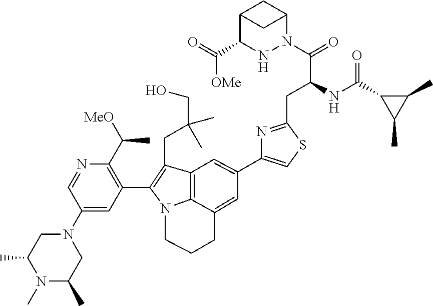

The title compound (303 mg, 67%) was prepared in a manner similar to that in example 35 step 4 from [3-(3-hydroxy-2,2-dimethylpropyl)-2-{2-[(1S)-1-methoxyethyl]-5-[(3R,5R)-3,4,5-trimethylpiperazin-1-yl]pyridin-3-yl}-1-azatricyclo[6.3.1.0^{4,12}]dodeca-2,4(12),5,7-tetraen-6-yl]boronic acid and methyl (4S)-2-[(2S)-3-(4-bromo-1,3-thiazol-2-yl)-2-{[(R,2R,3S)-2,3-dimethylcyclopropyl]formamido}propanoyl]-2,3-diazabicyclo[3.1.1]heptane-4-carboxylate. LC-MS (M+H)⁺=909.8.

### Step 6: (4S)-2-[(2S)-2-{[(1R,2R,3S)-2,3-dimethylcyclopropyl]formamido}-3-{4-[3-(3-hydroxy-2,2-dimethylpropyl)-2-{2-[(1S)-1-methoxyethyl]-5-[(3R,5R)-3,4,5-trimethylpiperazin-1-yl]pyridin-3-yl}-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic Acid

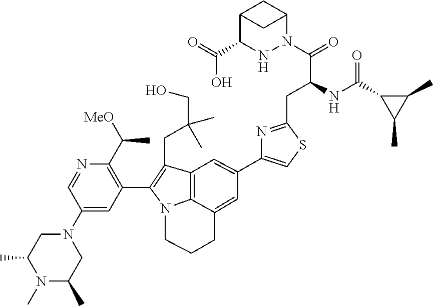

The title compound (297 mg, 99%) was prepared in a manner similar to that in example 35 step 5 from methyl (4S)-2-[(2S)-2-{[(1R,2R,3S)-2,3-dimethylcyclopropyl]formamido}-3-{4-[3-(3-hydroxy-2,2-dimethylpropyl)-2-{2-[(1S)-1-methoxyethyl]-5-[(3R,5R)-3,4,5-trimethylpiperazin-1-yl]pyridin-3-yl}-1-azatricyclo[6.3.1.0^{4,12}]dodeca-2,4(12),5,7-tetraen-6-yl]-1,3-thiazol-2-yl)propanoyl]-2,3-diazabicyclo[3.1.1]heptane-4-carboxylate. LC-MS (M+H)⁺=895.7.

### Step 7: (1R,2R,3S)—N-[(7S,13S,19M)-20-{2-[(1S)-1-methoxyethyl]-5-[(3R,5R)-3,4,5-trimethylpiperazin-1-yl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

Example 53 (97 mg, 33%) was prepared in a manner similar to that in example 35 step 6 from (4S)-2-[(2S)-2-{[(1R,2R,3S)-2,3-dimethylcyclopropyl]formamido}-3-{4-[3-(3-hydroxy-2,2-dimethylpropyl)-2-{2-[(1S)-1-methoxyethyl]-5-[(3R,5R)-3,4,5-trimethylpiperazin-1-yl]pyridin-3-yl}-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic acid. ¹H NMR (400 MHz, DMSO-d₆) δ 8.46-8.27 (m, 2H), 8.21 (s, 1H), 7.68 (s, 1H), 7.40 (s, 1H), 7.22-6.97 (m, 1H), 5.90 (d, J=11.0 Hz, 1H), 5.41-5.20 (m, 1H), 4.63 (d, J=11.0 Hz, 1H), 4.52-4.35 (m, 1H), 4.31-4.12 (m, 2H), 3.65-3.41 (m, 3H), 3.28-3.15 (m, 6H), 3.09 (dd, J=14.9, 7.3 Hz, 1H), 3.03-2.74 (m, 7H), 2.63-2.56 (m, 1H), 2.54-2.02 (m, 9H), 1.51 (t, J=9.4 Hz, 1H), 1.30 (d, J=6.0 Hz, 3H), 1.21-1.11 (m, 3H), 1.08-1.01 (m, 6H), 0.95 (d, J=6.3 Hz, 6H), 0.84 (s, 3H), 0.36 (s, 3H). LC-MS (M+H)⁺=877.8.

### Example 54: (1S,2S)—N-[(7S,13S,19M)-20-{2-[(1S)-1-methoxyethyl]-5-[(3R,5R)-3,4,5-trimethylpiperazin-1-yl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2-methylcyclopropane-1-carboxamide

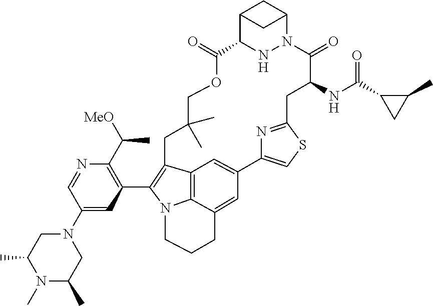

### Step 1: methyl (4S)-2-[(2S)-3-{4-[3-(3-hydroxy-2,2-dimethylpropyl)-2-{2-[(1S)-1-methoxyethyl]-5-[(3R,5R)-3,4,5-trimethylpiperazin-1-yl]pyridin-3-yl}-1-azatricyclo[6.3.1.0^{4,12}]dodeca-2,4(12),5,7-tetraen-6-yl]-1,3-thiazol-2-yl}-2-{[(1S,2S)-2-methylcyclopropyl]formamido}propanoyl]-2,3-diazabicyclo[3.1.1]heptane-4-carboxylate

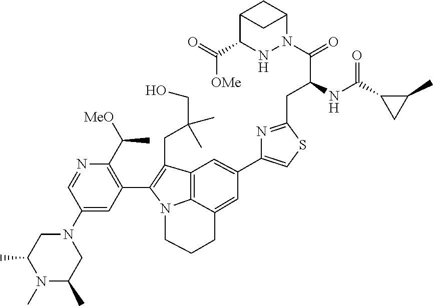

The title compound (303 mg, 62%) was prepared in a manner similar to that in example 35 step 4 from [3-(3-hydroxy-2,2-dimethylpropyl)-2-{2-[(1S)-1-methoxyethyl]-5-[(3R,5R)-3,4,5-trimethylpiperazin-1-yl]pyridin-3-yl}-1-azatricyclo[6.3.1.0^{4,12}]dodeca-2,4(12),5,7-tetraen-6-yl]boronic acid and methyl (4S)-2-[(2S)-3-(4-bromo-1,3-thiazol-2-yl)-2-{[(1S,2S)-2-methylcyclopropyl]formamido}propanoyl]-2,3-diazabicyclo[3.1.1]heptane-4-carboxylate. LC-MS (M+H)⁺=895.8.

### Step 2: (4S)-2-[(2S)-3-{4-[3-(3-hydroxy-2,2-dimethylpropyl)-2-{2-[(1S)-1-methoxyethyl]-5-[(3R,5R)-3,4,5-trimethylpiperazin-1-yl]pyridin-3-yl}-1-azatricyclo[6.3.1.0^{4,12}]dodeca-2,4(12),5,7-tetraen-6-yl]-1,3-thiazol-2-yl}-2-{[(1S,2S)-2-methylcyclopropyl]formamido}propanoyl]-2,3-diazabicyclo[3.1.1]heptane-4-carboxylic Acid

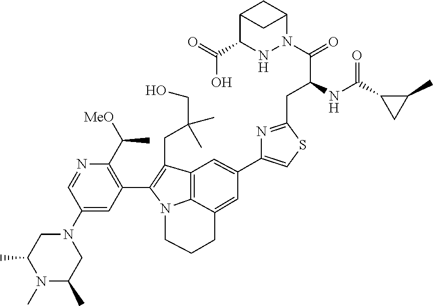

The title compound (271 mg, 99%) was prepared in a manner similar to that in example 35 step 5 from methyl (4S)-2-[(2S)-3-{4-[3-(3-hydroxy-2,2-dimethylpropyl)-2-(2-[(1S)-1-methoxyethyl]-5-[(3R,5R)-3,4,5-trimethylpiperazin-1-yl]pyridin-3-yl}-1-azatricyclo[6.3.1.0^{4,12}]dodeca-2,4(12),5,7-tetraen-6-yl]-1,3-thiazol-2-yl)-2-{[(1S,2S)-2-methylcyclopropyl]formamido}propanoyl]-2,3-diazabicyclo[3.1.1]heptane-4-carboxylate. LC-MS (M+H)⁺=881.6.

### Step 3: (1S,2S)—N-[(7S,13S,19M)-20-{2-[(1S)-1-methoxyethyl]-5-[(3R,5R)-3,4,5-trimethylpiperazin-1-yl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2-methylcyclopropane-1-carboxamide

Example 54 (74 mg, 28%) was prepared in a manner similar to that in example 35 step 6 from (4S)-2-[(2S)-3-{4-[3-(3-hydroxy-2,2-dimethylpropyl)-2-{2-[(1S)-1-methoxyethyl]-5-[(3R,5R)-3,4,5-trimethylpiperazin-1-yl]pyridin-3-yl}-1-azatricyclo[6.3.1.0^{4,12}]dodeca-2,4(12),5,7-tetraen-6-yl]-1,3-thiazol-2-yl}-2-{[(1S,2S)-2-methylcyclopropyl]formamido}propanoyl]-2,3-diazabicyclo[3.1.1]heptane-4-carboxylic acid. ¹H NMR (400 MHz, DMSO-d₆) δ 8.49 (d, J=8.9 Hz, 1H), 8.38 (d, J=2.9 Hz, 1H), 8.21 (s, 1H), 7.68 (s, 1H), 7.40 (s, 1H), 7.08 (d, J=3.0 Hz, 1H), 5.91 (d, J=11.0 Hz, 1H), 5.36 (t, J=8.1 Hz, 1H), 4.64 (d, J=11.0 Hz, 1H), 4.52-4.41 (m, 1H), 4.25-4.13 (m, 2H), 3.64-3.40 (m, 3H), 3.28-3.15 (m, 6H), 3.09 (dd, J=14.8, 7.3 Hz, 1H), 3.02-2.95 (m, 3H), 2.90-2.75 (m, 4H), 2.63-2.55 (m, 1H), 2.44-2.05 (m, 9H), 1.59-1.42 (m, 2H), 1.30 (d, J=6.0 Hz, 3H), 1.09-0.80 (m, 14H), 0.57-0.48 (m, 1H), 0.35 (s, 3H). LC-MS (M+H)⁺=863.8.

### Example 55: (1R,2R,3S)—N-[(7S,13S,19M)-20-{5-[(3R,5S)-4-cyclobutyl-3,5-dimethylpiperazin-1-yl]-2-[(1S)-1-methoxyethyl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

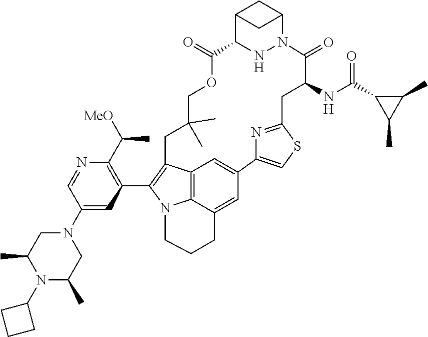

### Step 1: (2R,6S)-1-cyclobutyl-2,6-dimethylpiperazine 2,2,2-trifluoroacetate

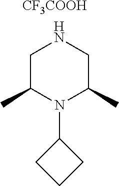

To a solution of tert-butyl (3S,5R)-3,5-dimethylpiperazine-1-carboxylate (5.0 g, 23.4 mmol) in DCM (100 mL) was added cyclobutanone (3.3 g, 46.7 mmol), AcOH (1.4 g, 23.4 mmol) and sodium triacetoxyborohydride (9.9 g, 46.7 mmol). The reaction mixture was stirred at rt for two days. The reaction mixture was quenched by the addition of a saturated aqueous solution of NaHCO₃ (20 mL). The mixture was then extracted with EA (50 mL×3). The organic layer was dried over Na₂SO₄, filtered and concentrated in vacuo to give a residue. The residue was filtered through a short pad of silica gel and eluted with DCM/MeOH=100:1. The filtrate was concentrated in vacuo to give a residue. The residue was dissolved in trifluoroacetic acid (25 mL) and the mixture was stirred at rt for 1 h. Volatiles were removed in vacuo to give the title compound, which was used in the next step without further purification. LC-MS (M+H)⁺=169.1.

### Step 2: 3-[(2M)-6-bromo-2-{5-[(3R,5S)-4-cyclobutyl-3,5-dimethylpiperazin-1-yl]-2-[(15)-1-methoxyethyl]pyridin-3-yl}-1-azatricyclo[6.3.1.0^{4,12}]dodeca-2,4(12),5,7-tetraen-3-yl]-2,2-dimethylpropan-1-ol

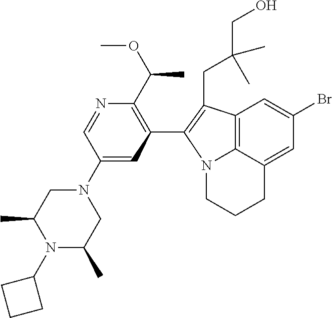

The title compound (300 mg, 14%) was prepared in a manner similar to that in example 20 step 1 from 3-[(2M)-6-bromo-2-{2-[(1S)-1-methoxyethyl]-5-(4,4,5,5-tetramethyl-1,3,2-dioxaborolan-2-yl)pyridin-3-yl}-1-azatricyclo[6.3.1.0^{4,12}]dodeca-2,4(12),5,7-tetraen-3-yl]-2,2-dimethylpropan-1-ol and (2R,6S)-1-cyclobutyl-2,6-dimethylpiperazine 2,2,2-trifluoroacetate. LC-MS (M+H)⁺=623.2/625.2.

### Step 3: [(2M)-2-{5-[(3R,5S)-4-cyclobutyl-3,5-dimethylpiperazin-1-yl]-2-[(1S)-1-methoxyethyl]pyridin-3-yl}-3-(3-hydroxy-2,2-dimethylpropyl)-1-azatricyclo[6.3.1.0^{4,12}]dodeca-2,4(12),5,7-tetraen-6-yl]boronic Acid

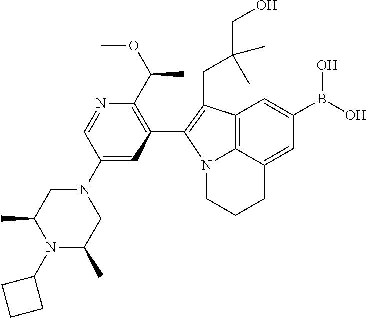

The title compound (300 mg, 14%) was prepared in a manner similar to that in example 35 step 3 from 3-[(2M)-6-bromo-2-{5-[(3R,5S)-4-cyclobutyl-3,5-dimethylpiperazin-1-yl]-2-[(1S)-1-methoxyethyl]pyridin-3-yl}-1-azatricyclo[6.3.1.0^{4,12}]dodeca-2,4(12),5,7-tetraen-3-yl]-2,2-dimethylpropan-1-ol. LC-MS (M+H)⁺=589.3.

### Step 4: methyl (4S)-2-[(2S)-3-{4-[(2M)-2-{5-[(3R,5S)-4-cyclobutyl-3,5-dimethylpiperazin-1-yl]-2-[(1S)-1-methoxyethyl]pyridin-3-yl}-3-(3-hydroxy-2,2-dimethylpropyl)-1-azatricyclo[6.3.1.0^{4,12}]dodeca-2,4(12),5,7-tetraen-6-yl]-1,3-thiazol-2-yl}-2-[(1R,2R,3S)-2,3-dimethylcyclopropyl]formamido}propanoyl]-2,3-diazabicyclo[3.1.1]heptane-4-carboxylate

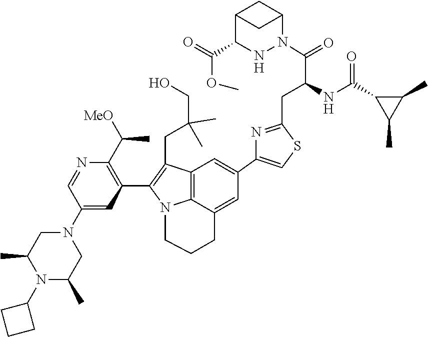

The title compound (340 mg, 75%) was prepared in a manner similar to that in example 35 step 4 from [(2M)-2-{5-[(3R,5S)-4-cyclobutyl-3,5-dimethylpiperazin-1-yl]-2-[(1S)-1-methoxyethyl]pyridin-3-yl}-3-(3-hydroxy-2,2-dimethylpropyl)-1-azatricyclo[6.3.1.0^{4,12}]dodeca-2,4(12),5,7-tetraen-6-yl]boronic acid and methyl (4S)-2-[(2S)-3-(4-bromo-1,3-thiazol-2-yl)-2-{[(1R,2R,3S)-2,3-dimethylcyclopropyl]formamido}propanoyl]-2,3-diazabicyclo[3.1.1]heptane-4-carboxylate. LC-MS (M+H)⁺=949.5.

### Step 5: (4S)-2-[(2S)-3-{4-[(2M)-2-{5-[(3R,5S)-4-cyclobutyl-3,5-dimethylpiperazin-1-yl]-2-[(1S)-1-methoxyethyl]pyridin-3-yl}-3-(3-hydroxy-2,2-dimethylpropyl)-1-azatricyclo[6.3.1.0^{4,12}]dodeca-2,4(12),5,7-tetraen-6-yl]-1,3-thiazol-2-yl}-2-{[(1R,2R,3S)-2,3-dimethylcyclopropyl]formamido}propanoyl]-2,3-diazabicyclo[3.1.1]heptane-4-carboxylic Acid

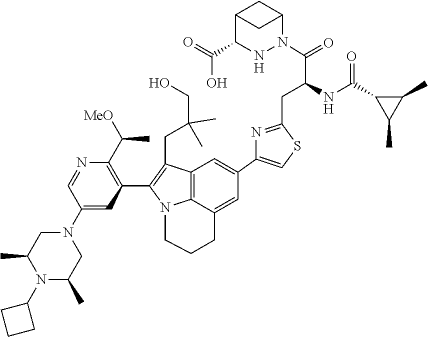

The title compound (270 mg, 81%) was prepared in a manner similar to that in example 35 step 5 from methyl (4S)-2-[(2S)-3-{4-[(2M)-2-{5-[(3R,5S)-4-cyclobutyl-3,5-dimethylpiperazin-1-yl]-2-[(1S)-1-methoxyethyl]pyridin-3-yl}-3-(3-hydroxy-2,2-dimethylpropyl)-1-azatricyclo[6.3.1.0^{4,12}]dodeca-2,4(12),5,7-tetraen-6-yl]-1,3-thiazol-2-yl}-2-{[(1R,2R,3S)-2,3-dimethylcyclopropyl]formamido}propanoyl]-2,3-diazabicyclo[3.1.1]heptane-4-carboxylate. LC-MS (M+H)⁺=935.5.

### Step 6: (1R,2R,3S)—N-[(7S,13S,19M)-20-{5-[(3R,5S)-4-cyclobutyl-3,5-dimethylpiperazin-1-yl]-2-[(1S)-1-methoxyethyl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

Example 55 (30 mg, 11%) was prepared in a manner similar to that in example 35 step 6 from (4S)-2-[(2S)-3-{4-[(2M)-2-{5-[(3R,5S)-4-cyclobutyl-3,5-dimethylpiperazin-1-yl]-2-[(1S)-1-methoxyethyl]pyridin-3-yl}-3-(3-hydroxy-2,2-dimethylpropyl)-1-azatricyclo[6.3.1.0^{4,12}]dodeca-2,4(12),5,7-tetraen-6-yl]-1,3-thiazol-2-yl}-2-{[(1R,2R,3S)-2,3-dimethylcyclopropyl]formamido}propanoyl]-2,3-diazabicyclo[3.1.1]heptane-4-carboxylic acid.

¹H NMR (400 MHz, DMSO-d6) δ 8.38 (d, J=8.9 Hz, 1H), 8.32 (d, J=2.9 Hz, 1H), 8.21 (s, 1H), 7.68 (s, 1H), 7.40 (s, 1H), 7.01 (d, J=2.9 Hz, 1H), 5.89 (d, J=11.0 Hz, 1H), 5.35 (t, J=8.3 Hz, 1H), 4.63 (d, J=11.0 Hz, 1H), 4.50-4.40 (m, 1H), 4.30-4.10 (m, 2H), 3.61-3.46 (m, 3H), 3.45-3.34 (m, 3H), 3.24-3.16 (m, 4H), 3.12-2.81 (m, 8H), 2.64-2.15 (m, 5H), 2.13-1.89 (m, 4H) 1.81-1.68 (m, 2H), 1.61-1.47 (m, 3H), 1.30 (d, J=6.0 Hz, 3H), 1.21-1.10 (m, 3H), 1.07-0.94 (m, 12H), 0.84 (s, 3H), 0.35 (s, 3H). LC-MS (M+H)⁺=917.5.

### Example 56: (1S,2S)—N-[(7S,13S,19M)-20-{5-[(3R,5S)-4-cyclobutyl-3,5-dimethylpiperazin-1-yl]-2-[(1S)-1-methoxyethyl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2-methylcyclopropane-1-carboxamide

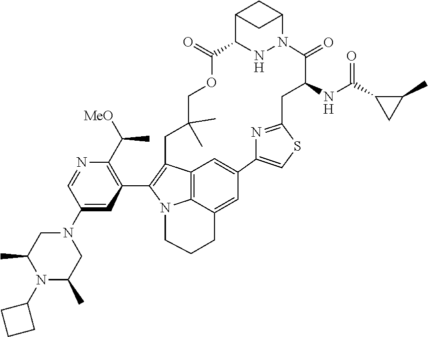

### Step 1: methyl (4S)-2-[(2S)-3-{4-[(2M)-2-{5-[(3R,5S)-4-cyclobutyl-3,5-dimethylpiperazin-1-yl]-2-[(1S)-1-methoxyethyl]pyridin-3-yl}-3-(3-hydroxy-2,2-dimethylpropyl)-1-azatricyclo[6.3.1.0^{4,12}]dodeca-2,4(12),5,7-tetraen-6-yl]-1,3-thiazol-2-yl}-2-{[(1S,2S)-2-methylcyclopropyl]formamido}propanoyl]-2,3-diazabicyclo[3.1.1]heptane-4-carboxylate

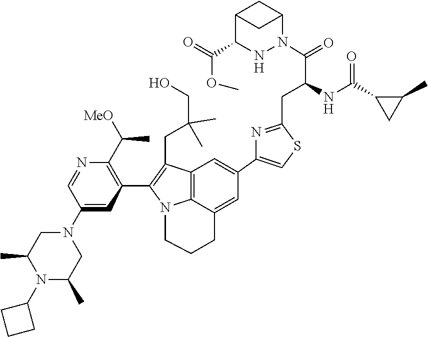

The title compound (330 mg, 75%) was prepared in a manner similar to that in example 35 step 4 from [(2M)-2-{5-[(3R,5S)-4-cyclobutyl-3,5-dimethylpiperazin-1-yl]-2-[(1S)-1-methoxyethyl]pyridin-3-yl}-3-(3-hydroxy-2,2-dimethylpropyl)-1-azatricyclo[6.3.1.0^{4,12}]dodeca-2,4(12),5,7-tetraen-6-yl]boronic acid and methyl (4S)-2-[(2S)-3-(4-bromo-1,3-thiazol-2-yl)-2-{[(1S,2S)-2-methylcyclopropyl]formamido}propanoyl]-2,3-diazabicyclo[3.1.1]heptane-4-carboxylate. LC-MS (M+H)⁺=935.5.

### Step 2: (4S)-2-[(2S)-3-{4-[(2M)-2-{5-[(3R,5S)-4-cyclobutyl-3,5-dimethylpiperazin-1-yl]-2-[(1S)-1-methoxyethyl]pyridin-3-yl}-3-(3-hydroxy-2,2-dimethylpropyl)-1-azatricyclo[6.3.1.0^{4,12}]dodeca-2,4(12),5,7-tetraen-6-yl]-1,3-thiazol-2-yl}-2-{[(1S,2S)-2-methylcyclopropyl]formamido}propanoyl]-2,3-diazabicyclo[3.1.1]heptane-4-carboxylic Acid

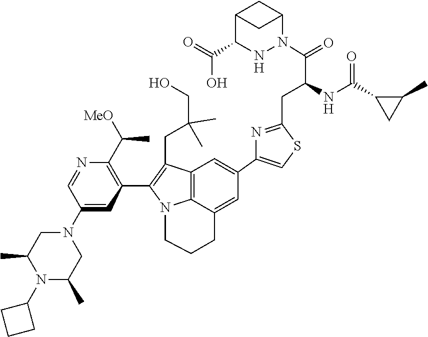

The title compound (180 mg, 55%) was prepared in a manner similar to that in example 35 step 5 from methyl (4S)-2-[(2S)-3-{4-[(2M)-2-{5-[(3R,5S)-4-cyclobutyl-3,5-dimethylpiperazin-1-yl]-2-[(1S)-1-methoxyethyl]pyridin-3-yl}-3-(3-hydroxy-2,2-dimethylpropyl)-1-azatricyclo[6.3.1.0^{4,12}]dodeca-2,4(12),5,7-tetraen-6-yl]-1,3-thiazol-2-yl}-2-{[(1S,2S)-2-methylcyclopropyl]formamido}propanoyl]-2,3-diazabicyclo[3.1.1]heptane-4-carboxylate. LC-MS (M+H)⁺=921.5.

### Step 3: (1S,2S)—N-[(7S,13S,19M)-20-{5-[(3R,5S)-4-cyclobutyl-3,5-dimethylpiperazin-1-yl]-2-[(1S)-1-methoxyethyl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2-methylcyclopropane-1-carboxamide

Example 56 (30 mg, 17%) was prepared in a manner similar to that in example 35 step 6 from (4S)-2-[(2S)-3-{4-[(2M)-2-{5-[(3R,5S)-4-cyclobutyl-3,5-dimethylpiperazin-1-yl]-2-[(1S)-1-methoxyethyl]pyridin-3-yl}-3-(3-hydroxy-2,2-dimethylpropyl)-1-azatricyclo[6.3.1.0^{4,12}]dodeca-2,4(12),5,7-tetraen-6-yl]-1,3-thiazol-2-yl}-2-{[(1S,2S)-2-methylcyclopropyl]formamido}propanoyl]-2,3-diazabicyclo[3.1.1]heptane-4-carboxylic acid.

¹H NMR (400 MHz, DMSO-d₆) δ 8.50 (d, J=8.9 Hz, 1H), 8.32 (d, J=2.3 Hz, 1H), 8.21 (s, 1H), 7.68 (s, 1H), 7.40 (s, 1H), 7.01 (d, J=2.9 Hz, 1H), 5.91 (d, J=11.1 Hz, 1H), 5.44-5.25 (m, 1H), 4.64 (d, J=11.0 Hz, 1H), 4.57-4.37 (m, 1H), 4.29-4.15 (m, 2H), 3.60-3.47 (m, 3H), 3.46-3.34 (m, 3H), 3.27-3.16 (m, 4H), 3.12-2.81 (m, 8H), 2.64-2.05 (m, 7H), 1.99-1.89 (m, 2H), 1.79-1.68 (m, 2H), 1.62-1.41 (m, 4H), 1.29 (d, J=6.0 Hz, 3H), 1.10-0.96 (m, 10H), 0.92-0.78 (m, 4H), 0.56-0.48 (m, 1H), 0.34 (s, 3H). LC-MS (M+H)⁺=903.4.

Atrop-57 & Example 57: (1R,2R,3S)—N-[(7S,13S,19P)-20-{5-[(3R,5S)-4-cyclopropyl-3,5-dimethylpiperazin-1-yl]-2-[(1S)-1-methoxyethyl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide and (1R,2R,3S)—N-[(7S,13S,19M)-20-{5-[(3R,5S)-4-cyclopropyl-3,5-dimethylpiperazin-1-yl]-2-[(1S)-1-methoxyethyl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

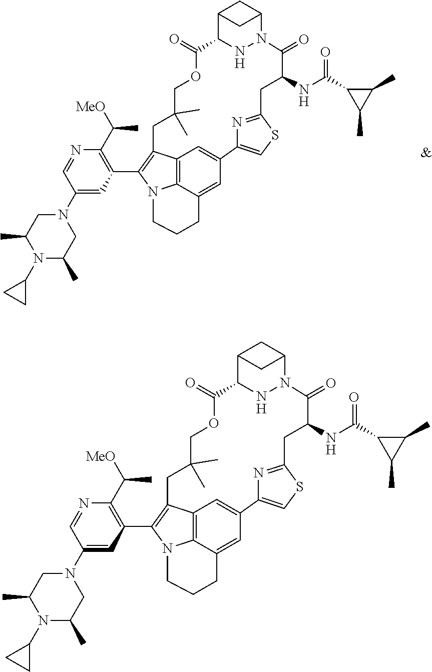

### Step 1: tert-butyl (3R,5S)-4-cyclopropyl-3,5-dimethylpiperazine-1-carboxylate

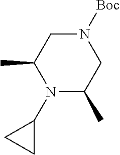 [SMILES: C[C@@H]1CN(C(=O)OC(C)(C)C)C[C@H](C)N1C1CC1]

To a solution of tert-butyl (3R,5S)-3,5-dimethylpiperazine-1-carboxylate (5.0 g, 23.4 mmol) in THF (50 mL) and MeOH (50 mL) was added (1-ethoxycyclopropoxy)trimethylsilane (12.2 g, 70.0 mmol), AcOH (14 g, 234 mmol) and sodium cyanoborohydride (2.9 g, 46.7 mmol). The mixture was then stirred for 40 h at 70° C. under nitrogen atmosphere. The mixture was cooled to room temperature, diluted with EA (100 mL), washed with NaHCO₃ (aq., 80 mL), brine (50 mL), dried over Na₂SO₄, filtered and concentrated under vacuum. The title product was used in the next step without purification. LC-MS (M+H)⁺=255.3.

### Step 2: (2R,6S)-1-cyclopropyl-2,6-dimethylpiperazine

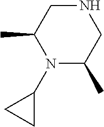

A solution of tert-butyl (3R,5S)-4-cyclopropyl-3,5-dimethylpiperazine-1-carboxylate in HCl (4 N in dioxane, 25 mL) was stirred for 2 h at room temperature and then concentrated under vacuum to give a residue. To this residue, NH₃ (7 N in MeOH, 15 mL) was added, and the mixture was stirred for 10 min at room temperature and concentrated under vacuum. The residue was purified by silica gel column chromatograph (MeOH:DCM=1:5) to give the title compound (2.2 g, 61% over two steps). LC-MS (M+H)⁺=155.2.

### Step 3: 3-(6-bromo-2-{2-[(1S)-1-methoxyethyl]-5-(4,4,5,5-tetramethyl-1,3,2-dioxaborolan-2-yl)pyridin-3-yl}-1-azatricyclo[6.3.1.0^{4,12}]dodeca-2,4(12),5,7-tetraen-3-yl)-2,2-dimethylpropan-1-ol

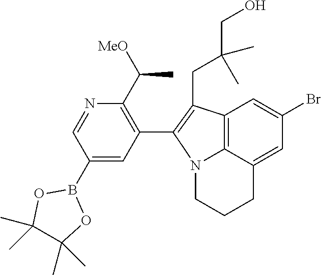 [SMILES: CO[C@@H](C)c1ncc(B2OC(C)(C)C(C)(C)O2)cc1-c1c(CC(C)(C)CO)c2cc(Br)cc3c2n1CCC3]

The title compound (500 mg, 90%) was prepared in a manner similar to that in example 50 step 2 from 3-(6-bromo-2-{2-[(1S)-1-methoxyethyl]pyridin-3-yl}-1-azatricyclo[6.3.1.0^{4,12}]dodeca-2,4(12),5,7-tetraen-3-yl)-2,2-dimethylpropan-1-ol. LC-MS (M-pinacol+2H₂O+H)⁺=501.3/503.3.

### Step 4: 3-(6-bromo-2-{5-[(3R,5S)-4-cyclopropyl-3,5-dimethylpiperazin-1-yl]-2-[(1S)-1-methoxyethyl]pyridin-3-yl}-1-azatricyclo[6.3.1.0^{4,12}]dodeca-2,4(12),5,7-tetraen-3-yl)-2,2-dimethylpropan-1-ol

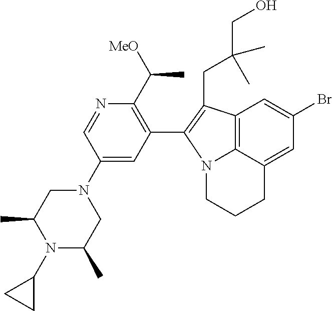 [SMILES: CO[C@@H](C)c1ncc(N2C[C@@H](C)N(C3CC3)[C@@H](C)C2)cc1-c1c(CC(C)(C)CO)c2cc(Br)cc3c2n1CCC3]

The title compound (500 mg, 60%) was prepared in a manner similar to that in example 20 step 1 from 3-(6-bromo-2-{2-[(1S)-1-methoxyethyl]-5-(4,4,5,5-tetramethyl-1,3,2-dioxaborolan-2-yl)pyridin-3-yl}-1-azatricyclo[6.3.1.0^{4,12}]dodeca-2,4(12),5,7-tetraen-3-yl)-2,2-dimethylpropan-1-ol and (2R,6S)-1-cyclopropyl-2,6-dimethylpiperazine. LC-MS (M+H)⁺=609.5/611.5.

### Step 5: (2-{5-[(3R,5S)-4-cyclopropyl-3,5-dimethylpiperazin-1-yl]-2-[(1S)-1-methoxyethyl]pyridin-3-yl}-3-(3-hydroxy-2,2-dimethylpropyl)-1-azatricyclo[6.3.1.0^{4,12}]dodeca-2,4(12),5,7-tetraen-6-yl)boronic Acid

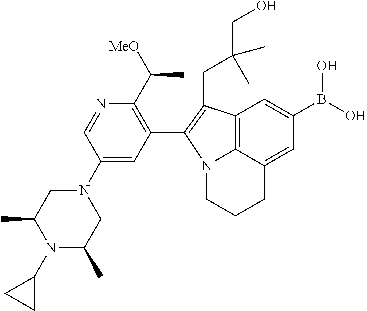 [SMILES: CO[C@@H](C)c1ncc(N2C[C@@H](C)N(C3CC3)[C@@H](C)C2)cc1-c1c(CC(C)(C)CO)c2cc(B(O)O)cc3c2n1CCC3]

The title compound (400 mg, 85%) was prepared in a manner similar to that in example 35 step 3 from 3-(6-bromo-2-{5-[(3R,5S)-4-cyclopropyl-3,5-dimethylpiperazin-1-yl]-2-[(1S)-1-methoxyethyl]pyridin-3-yl}-1-azatricyclo[6.3.1.0^{4,12}]dodeca-2,4(12),5,7-tetraen-3-yl)-2,2-dimethylpropan-1-ol. LC-MS (M+H)⁺=575.5.

### Step 6: methyl (4S)-2-[(2S)-3-[4-(2-{5-[(3R,5S)-4-cyclopropyl-3,5-dimethylpiperazin-1-yl]-2-[(1S)-1-methoxyethyl]pyridin-3-yl}-3-(3-hydroxy-2,2-dimethylpropyl)-1-azatricyclo[6.3.1.0^{4,12}]dodeca-2,4(12),5,7-tetraen-6-yl)-1,3-thiazol-2-yl]-2-{[(1R,2R,3S)-2,3-dimethylcyclopropyl]formamido}propanoyl]-2,3-diazabicyclo[3.1.1]heptane-4-carboxylate

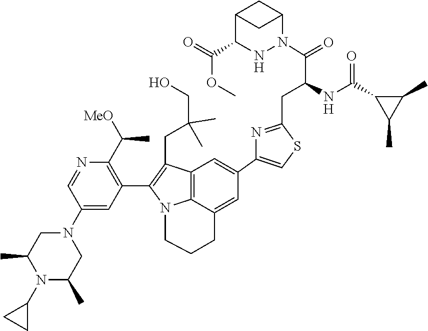

The title compound (250 mg, 77%) was prepared in a manner similar to that in example 35 step 4 from (2-{5-[(3R,5S)-4-cyclopropyl-3,5-dimethylpiperazin-1-yl]-2-[(1S)-1-methoxyethyl]pyridin-3-yl}-3-(3-hydroxy-2,2-dimethylpropyl)-1-azatricyclo[6.3.1.0^{4,12}]dodeca-2,4(12),5,7-tetraen-6-yl)boronic acid and methyl (4S)-2-[(2S)-3-(4-bromo-1,3-thiazol-2-yl)-2-{[(R,2R,3S)-2,3-dimethylcyclopropyl]formamido}propanoyl]-2,3-diazabicyclo[3.1.1]heptane-4-carboxylate. LC-MS (M+H)⁺=935.6.

### Step 7: (4S)-2-[(2S)-3-[4-(2-{5-[(3R,5S)-4-cyclopropyl-3,5-dimethylpiperazin-1-yl]-2-[(1S)-1-methoxyethyl]pyridin-3-yl}-3-(3-hydroxy-2,2-dimethylpropyl)-1-azatricyclo[6.3.1.0^{4,12}]dodeca-2,4(12),5,7-tetraen-6-yl)-1,3-thiazol-2-yl]-2-{[(1R,2R,3S)-2,3-dimethylcyclopropyl]formamido}propanoyl]-2,3-diazabicyclo[3.1.1]heptane-4-carboxylic Acid

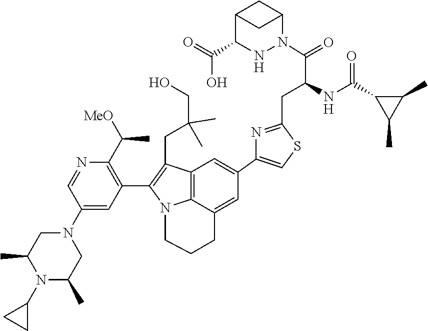

The title compound (170 mg, 69%) was prepared in a manner similar to that in example 35 step 5 from methyl (4S)-2-[(2S)-3-[4-(2-{5-[(3R,5S)-4-cyclopropyl-3,5-dimethylpiperazin-1-yl]-2-[(1S)-1-methoxyethyl]pyridin-3-yl}-3-(3-hydroxy-2,2-dimethylpropyl)-1-azatricyclo[6.3.1.0^{4,12}]dodeca-2,4(12),5,7-tetraen-6-yl)-1,3-thiazol-2-yl]-2-{[(1R,2R,3S)-2,3-dimethylcyclopropyl]formamido}propanoyl]-2,3-diazabicyclo[3.1.1]heptane-4-carboxylate. LC-MS (M+H)⁺=921.6.

### Step 8: (1R,2R,3S)—N-[(7S,13S,19P)-20-{5-[(3R,5S)-4-cyclopropyl-3,5-dimethylpiperazin-1-yl]-2-[(1S)-1-methoxyethyl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide and (1R,2R,3S)—N-[(7S,13S,19M)-20-{5-[(3R,5S)-4-cyclopropyl-3,5-dimethylpiperazin-1-yl]-2-[(1S)-1-methoxyethyl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

Atrop-57 (P-isomer, 20 mg, 17%) and example 57 (M-isomer, 25 mg, 21%) were prepared in a manner similar to that in example 35 step 6 from (4S)-2-[(2S)-3-[4-(2-{5-[(3R,5S)-4-cyclopropyl-3,5-dimethylpiperazin-1-yl]-2-[(1S)-1-methoxyethyl]pyridin-3-yl}-3-(3-hydroxy-2,2-dimethylpropyl)-1-azatricyclo[6.3.1.0^{4,12}]dodeca-2,4(12),5,7-tetraen-6-yl)-1,3-thiazol-2-yl]-2-{[(1R,2R,3S)-2,3-dimethylcyclopropyl]formamido}propanoyl]-2,3-diazabicyclo[3.1.1]heptane-4-carboxylic acid.

Atrop-57: ¹H NMR (400 MHz, DMSO-d6) δ 8.39 (s, 2H), 8.24 (s, 1H), 7.69 (s, 1H), 7.40 (s, 1H), 7.32 (s, 1H), 5.84 (d, J=12.0 Hz, 1H), 5.28 (t, J=8.0 Hz, 1H), 4.71 (d, J=12.0 Hz, 1H), 4.50-4.43 (m, 1H), 3.95-3.80 (m, 2H), 3.65-3.55 (m, 3H), 3.53-3.48 (m, 1H), 3.47-3.40 (m, 1H), 3.23-3.16 (m, 1H), 3.14-3.07 (m, 1H), 3.05 (s, 3H), 3.00-2.91 (m, 3H), 2.67-2.55 (m, 4H), 2.43-2.27 (m, 4H), 2.20-2.05 (m, 3H), 2.02-1.92 (m, 1H), 1.59 (t, J=8.0 Hz, 1H), 1.42-1.35 (m, 1H), 1.25-1.16 (m, 7H), 1.15-1.12 (m, 2H), 1.11-1.02 (m, 9H), 0.90 (s, 3H), 0.55-0.46 (m, 5H), 0.39 (s, 2H). LC-MS (M+H)⁺=903.7.

Example 57: ¹H NMR (400 MHz, DMSO-d6) δ 8.48-8.32 (m, 2H), 8.21 (s, 1H), 7.68 (s, 1H), 7.40 (s, 1H), 7.11 (d, J=2.9 Hz, 1H), 5.89 (d, J=11.0 Hz, 1H), 5.51-5.26 (m, 1H), 4.63 (d, J=11.0 Hz, 1H), 4.55-4.36 (m, 1H), 4.33-4.09 (m, 2H), 3.74-3.44 (m, 5H), 3.26-3.14 (m, 4H), 3.09 (dd, J=14.8, 7.3 Hz, 1H), 3.02-2.80 (m, 3H), 2.67-2.00 (m, 11H), 1.51 (t, J=9.4 Hz, 1H), 1.41-1.34 (m, 1H), 1.31 (d, J=6.1 Hz, 3H), 1.20-1.11 (m, 9H), 1.09-0.99 (m, 6H), 0.85 (s, 3H), 0.54-0.49 (m, 2H), 0.43-0.33 (m, 5H). LC-MS (M+H)⁺=903.7.

### Example 58: (1S,2S)—N-[(7S,13S,19M)-20-{5-[(3R,5S)-4-cyclopropyl-3,5-dimethylpiperazin-1-yl]-2-[(1S)-1-methoxyethyl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2-methylcyclopropane-1-carboxamide

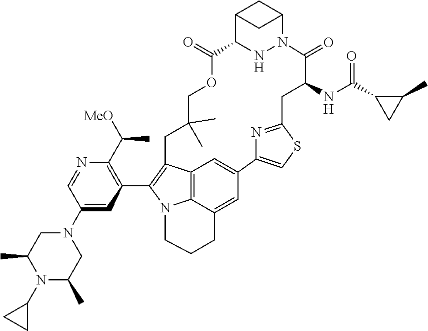

### Step 1: 3-[(2M)-6-bromo-2-{5-[(3R,5S)-4-cyclopropyl-3,5-dimethylpiperazin-1-yl]-2-[(1S)-1-methoxyethyl]pyridin-3-yl}-1-azatricyclo[6.3.1.0^{4,12}]dodeca-2,4(12),5,7-tetraen-3-yl]-2,2-dimethylpropan-1-ol

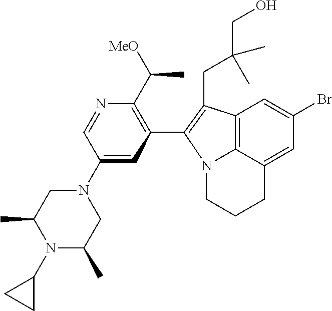

The title compound (200 mg, 48%) was prepared in a manner similar to that in example 20 step 1 from 3-[(2M)-6-bromo-2-{2-[(1S)-1-methoxyethyl]-5-(4,4,5,5-tetramethyl-1,3,2-dioxaborolan-2-yl)pyridin-3-yl}-1-azatricyclo[6.3.1.0^{4,12}]dodeca-2,4(12),5,7-tetraen-3-yl]-2,2-dimethylpropan-1-ol and (2R,6S)-1-cyclopropyl-2,6-dimethylpiperazine. LC-MS (M+H)⁺=609.5/611.5.

### Step 2: [(2M)-2-{5-[(3R,5S)-4-cyclopropyl-3,5-dimethylpiperazin-1-yl]-2-[(1S)-1-methoxyethyl]pyridin-3-yl}-3-(3-hydroxy-2,2-dimethylpropyl)-1-azatricyclo[6.3.1.0^{4,12}]dodeca-2,4(12),5,7-tetraen-6-yl]boronic Acid

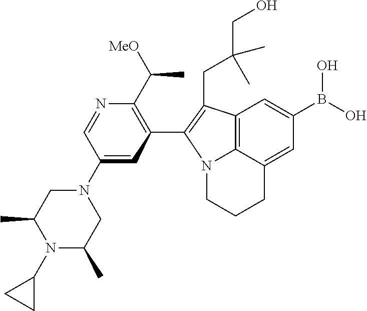

The title compound (180 mg, 95%) was prepared in a manner similar to that in example 35 step 3 from 3-[(2M)-6-bromo-2-{5-[(3R,5S)-4-cyclopropyl-3,5-dimethylpiperazin-1-yl]-2-[(1S)-1-methoxyethyl]pyridin-3-yl}-1-azatricyclo[6.3.1.0^{4,12}]dodeca-2,4(12),5,7-tetraen-3-yl]-2,2-dimethylpropan-1-ol. LC-MS (M+H)⁺=575.5.

### Step 3: methyl (4S)-2-[(2S)-3-{4-[(2M)-2-{5-[(3R,5S)-4-cyclopropyl-3,5-dimethylpiperazin-1-yl]-2-[(1S)-1-methoxyethyl]pyridin-3-yl}-3-(3-hydroxy-2,2-dimethylpropyl)-1-azatricyclo[6.3.1.0^{4,12}]dodeca-2,4(12),5,7-tetraen-6-yl]-1,3-thiazol-2-yl}-2-{[(1S,2S)-2-methylcyclopropyl]formamido}propanoyl]-2,3-diazabicyclo[3.1.1]heptane-4-carboxylate

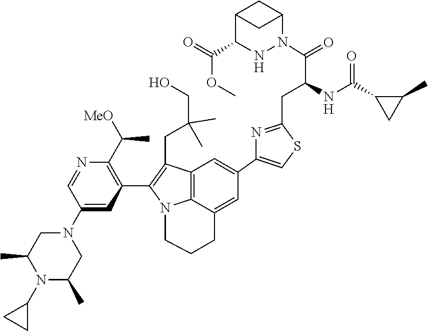

The title compound (250 mg, 69%) was prepared in a manner similar to that in example 35 step 4 from [(2M)-2-{5-[(3R,5S)-4-cyclopropyl-3,5-dimethylpiperazin-1-yl]-2-[(1S)-1-methoxyethyl]pyridin-3-yl}-3-(3-hydroxy-2,2-dimethylpropyl)-1-azatricyclo[6.3.1.0^{4,12}]dodeca-2,4(12),5,7-tetraen-6-yl]boronic acid and methyl (4S)-2-[(2S)-3-(4-bromo-1,3-thiazol-2-yl)-2-{[(1S,2S)-2-methylcyclopropyl]formamido}propanoyl]-2,3-diazabicyclo[3.1.1]heptane-4-carboxylate. LC-MS (M+H)⁺=921.6.

### Step 4: (4S)-2-[(2S)-3-{4-[(2M)-2-{5-[(3R,5S)-4-cyclopropyl-3,5-dimethylpiperazin-1-yl]-2-[(1S)-1-methoxyethyl]pyridin-3-yl}-3-(3-hydroxy-2,2-dimethylpropyl)-1-azatricyclo[6.3.1.0^{4,12}]dodeca-2,4(12),5,7-tetraen-6-yl]-1,3-thiazol-2-yl}-2-{[(1S,2S)-2-methylcyclopropyl]formamido}propanoyl]-2,3-diazabicyclo[3.1.1]heptane-4-carboxylic Acid

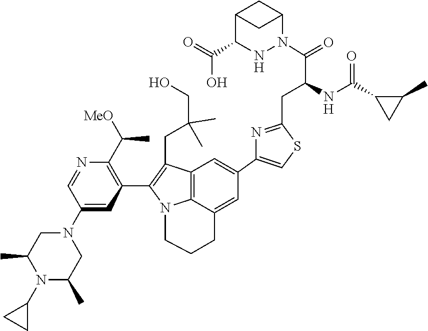

The title compound (185 mg, 75%) was prepared in a manner similar to that in example 35 step 5 from methyl (4S)-2-[(2S)-3-{4-[(2M)-2-{5-[(3R,5S)-4-cyclopropyl-3,5-dimethylpiperazin-1-yl]-2-[(1S)-1-methoxyethyl]pyridin-3-yl}-3-(3-hydroxy-2,2-dimethylpropyl)-1-azatricyclo[6.3.1.0^{4,12}]dodeca-2,4(12),5,7-tetraen-6-yl]-1,3-thiazol-2-yl}-2-{[(1S,2S)-2-methylcyclopropyl]formamido}propanoyl]-2,3-diazabicyclo[3.1.1]heptane-4-carboxylate. LC-MS (M+H)⁺=907.6.

### Step 5: (1S,2S)—N-[(7S,13S,19M)-20-{5-[(3R,5S)-4-cyclopropyl-3,5-dimethylpiperazin-1-yl]-2-[(1S)-1-methoxyethyl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2-methylcyclopropane-1-carboxamide

Example 58 (48 mg, 49%) was prepared in a manner similar to that in example 35 step 6 from (4S)-2-[(2S)-3-{4-[(2M)-2-{5-[(3R,5S)-4-cyclopropyl-3,5-dimethylpiperazin-1-yl]-2-[(1S)-1-methoxyethyl]pyridin-3-yl}-3-(3-hydroxy-2,2-dimethylpropyl)-1-azatricyclo[6.3.1.0^{4,12}]dodeca-2,4(12),5,7-tetraen-6-yl]-1,3-thiazol-2-yl}-2-{[(1S,2S)-2-methylcyclopropyl]formamido}propanoyl]-2,3-diazabicyclo[3.1.1]heptane-4-carboxylic acid.

¹H NMR (400 MHz, DMSO-d6) δ 8.50 (d, J=8.9 Hz, 1H), 8.39 (d, J=2.8 Hz, 1H), 8.21 (s, 1H), 7.68 (s, 1H), 7.40 (s, 1H), 7.11 (d, J=2.9 Hz, 1H), 5.91 (d, J=11.0 Hz, 1H), 5.46-5.27 (m, 1H), 4.64 (d, J=11.0 Hz, 1H), 4.54-4.38 (m, 1H), 4.28-4.08 (m, 2H), 3.67-3.38 (m, 5H), 3.26-3.15 (m, 4H), 3.09 (dd, J=14.9, 7.3 Hz, 1H), 3.02-2.79 (m, 3H), 2.67-2.02 (m, 11H), 1.52 (t, J=9.3 Hz, 1H), 1.48-1.35 (m, 2H), 1.30 (d, J=6.1 Hz, 3H), 1.23-0.80 (m, 14H), 0.58-0.48 (m, 3H), 0.44-0.28 (m, 5H). LC-MS (M+H)⁺=889.6.

### Example 59: (1R,2R,3S)—N-[(7S,13S,19M)-20-{5-[(6S)-5,6-dimethyl-5,8-diazaspiro[3.5]nonan-8-yl]-2-[(1S)-1-methoxyethyl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

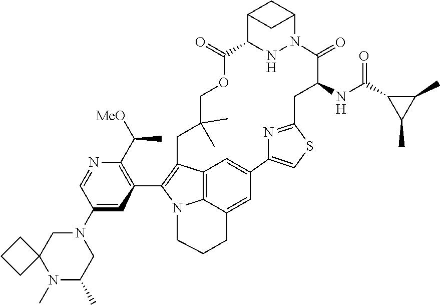

### Step 1: tert-butyl N-[1-({[(4-methoxyphenyl)methyl]amino}methyl)cyclobutyl]carbamate

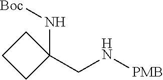

A mixture of tert-butyl N-[1-(aminomethyl)cyclobutyl]carbamate (5.0 g, 25 mmol), and 4-methoxybenzaldehyde (8.16 g, 59.9 mmol) in AcOH (1 mL) and MeOH (30 mL) was stirred at room temperature for 30 min. NaBH₄ (2.46 g, 65 mmol) was added to the mixture portion-wise. The mixture was stirred at room temperature for 2 h. The mixture was diluted with water (60 mL) and extracted with EA (50 mL×3). The combined organic layers were washed with brine (40 mL), dried over Na₂SO₄, filtered and concentrated. The residue was purified by column chromatography (DCM/MeOH=20/1) to give the title product (4.7 g, 59%). LC-MS (M+H)⁺=321.3.

### Step 2: tert-butyl N-(1-{[(2R)-2-chloro-N-[(4-methoxyphenyl)methyl]propanamido]methyl}cyclobutylcarbamate

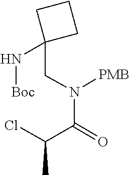

A mixture of tert-butyl N-[1-({[(4-methoxyphenyl)methyl]amino}methyl)cyclobutyl]carbamate (4.7 g, 15 mmol), (2R)-2-chloropropanoic acid (1.8 g, 16.6 mmol), HATU (6.7 g, 17.6 mmol) and DIPEA (3.8 g, 29.4 mmol) in DMF (80 mL) was stirred at room temperature for 2 h. The mixture was then diluted with water (160 mL) and extracted with EA (60 mL×3). The combined organic layers were washed with brine (50 mL), dried over Na₂SO₄, filtered and concentrated under vacuum. The residue was purified by column chromatography (DCM/MeOH=40/1) to give the title compound (4.7 g, 76%). LC-MS (M+H)⁺=411.3.

### Step 3: (2R)—N-[(1-aminocyclobutyl)methyl]-2-chloro-N-[(4-methoxyphenyl)methyl]propanamide Hydrochloride

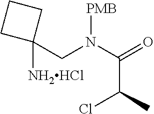

A mixture of tert-butyl N-(1-{[(2R)-2-chloro-N-[(4-methoxyphenyl)methyl]propanamido]methyl}cyclobutyl)carbamate (4.6 g, 11 mmol) and HCl in dioxane (4 N, 20 mL) in DCM (40 mL) was stirred at 20° C. for 2 h. Volatiles were removed under vacuum to give the title compound (3.5 g, 100%). LC-MS (M+H)⁺=311.3.

### Step 4: (6S)-8-[(4-methoxyphenyl)methyl]-6-methyl-5,8-diazaspiro[3.5]nonan-7-one

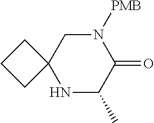

A mixture of (2R)—N-[(1-aminocyclobutyl)methyl]-2-chloro-N-[(4-methoxyphenyl)methyl]propanamide hydrochloride (3.5 g, 11 mmol) and DIPEA (2.9 g, 22.4 mmol) in DMF (50 mL) was stirred at 50° C. for 3 h. The mixture was diluted with water (150 mL) and extracted with EA (60 mL×3). The combined organic layers were washed with brine (50 mL), dried over Na₂SO₄, filtered and concentrated under vacuum. The residue was purified by column chromatography (DCM/MeOH=40/1) to give the title compound (3.0 g, 97%). LC-MS (M+H)⁺=275.3.

### Step 5: (6S)-8-[(4-methoxyphenyl)methyl]-6-methyl-5,8-diazaspiro[3.5]nonane

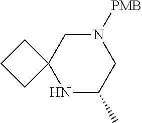

A mixture of (6S)-8-[(4-methoxyphenyl)methyl]-6-methyl-5,8-diazaspiro[3.5]nonan-7-one (3.0 g, 10.9 mmol) and borane (55 mL, 1 N in THF, 55 mmol) in THF (30 mL) was stirred at 80° C. for 20 h. The mixture was then cooled to room temperature. MeOH (5 mL) was added slowly, followed by the addition of HCl (4 N in dioxane, 20 mL). The mixture was concentrated under vacuum to give the title compound (2.5 g, 88%). LC-MS (M+H)⁺=261.3.

### Step 6: (6S)-8-[(4-methoxyphenyl)methyl]-5,6-dimethyl-5,8-diazaspiro[3.5]nonane

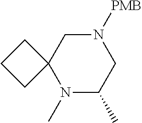

To a mixture of (6S)-8-[(4-methoxyphenyl)methyl]-6-methyl-5,8-diazaspiro[3.5]nonane (2.5 g, 9.1 mmol) and formaldehyde (4 mL, 37% in water) in MeOH (30 mL), sodium cyanoborohydride (1.1 g, 17.5 mmol) was added in portions. The mixture was stirred at room temperature for 5 h. The mixture was diluted with water (100 mL) and extracted with EA (50 mL×3). The combined organic layers were washed with brine (50 mL), dried over Na₂SO₄, filtered and concentrated under vacuum. The residue was purified by column chromatography (DCM/MeOH=20/1) to give the title compound (2.0 g, 84%). LC-MS (M+H)⁺=275.3.

### Step 7: (6S)-5,6-dimethyl-5,8-diazaspiro[3.5]nonane

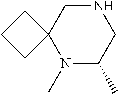

A mixture of (6S)-8-[(4-methoxyphenyl)methyl]-5,6-dimethyl-5,8-diazaspiro[3.5]nonane (2 g, 7.3 mmol) and Pd/C (0.2 g) in isopropanol (30 mL) was stirred at 70° C. under hydrogen atmosphere for 20 h. The mixture was cooled to room temperature and filtered through a short pad of celite. The filtrate was concentrated under vacuum to give the title compound (0.7 g, 60%). LC-MS (M+H)⁺=155.3.

### Step 8: 3-[(2M)-6-bromo-2-{5-[(6S)-5,6-dimethyl-5,8-diazaspiro[3.5]nonan-8-yl]-2-[(1S)-1-methoxyethyl]pyridin-3-yl}-1-azatricyclo[6.3.1.0^{4,12}]dodeca-2,4(12),5,7-tetraen-3-yl]-2,2-dimethylpropan-1-ol

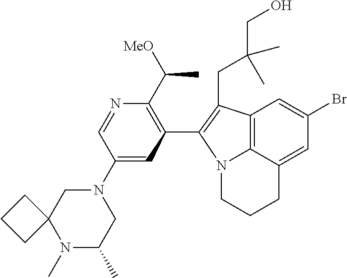

The title compound (430 mg, 35%) was prepared in a manner similar to that in example 20 step 1 from 3-[(2M)-6-bromo-2-{2-[(1S)-1-methoxyethyl]-5-(4,4,5,5-tetramethyl-1,3,2-dioxaborolan-2-yl)pyridin-3-yl}-1-azatricyclo[6.3.1.0^{4,12}]dodeca-2,4(12),5,7-tetraen-3-yl]-2,2-dimethylpropan-1-ol and (6S)-5,6-dimethyl-5,8-diazaspiro[3.5]nonane. LC-MS (M+H)⁺=609.4/611.3.

### Step 9: [(2M)-2-{5-[(6S)-5,6-dimethyl-5,8-diazaspiro[3.5]nonan-8-yl]-2-[(1S)-1-methoxyethyl]pyridin-3-yl}-3-(3-hydroxy-2,2-dimethylpropyl)-1-azatricyclo[6.3.1.0^{4,12}]dodeca-2,4(12),5,7-tetraen-6-yl]boronic Acid

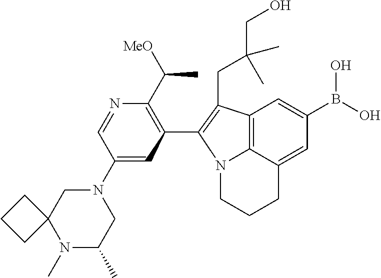

The title compound (40 mg, 99%) was prepared in a manner similar to that in example 35 step 3 from 3-[(2M)-6-bromo-2-{5-[(6S)-5,6-dimethyl-5,8-diazaspiro[3.5]nonan-8-yl]-2-[(1S)-1-methoxyethyl]pyridin-3-yl}-1-azatricyclo[6.3.1.0^{4,12}]dodeca-2,4(12),5,7-tetraen-3-yl]-2,2-dimethylpropan-1-ol. LC-MS (M+H)⁺=575.3.

### Step 10: methyl (4S)-2-[(2S)-2-{[(tert-butoxy)carbonyl]amino}-3-{4-[(2M)-2-{5-[(6S)-5,6-dimethyl-5,8-diazaspiro[3.5]nonan-8-yl]-2-[(1S)-1-methoxyethyl]pyridin-3-yl}-3-(3-hydroxy-2,2-dimethylpropyl)-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylate

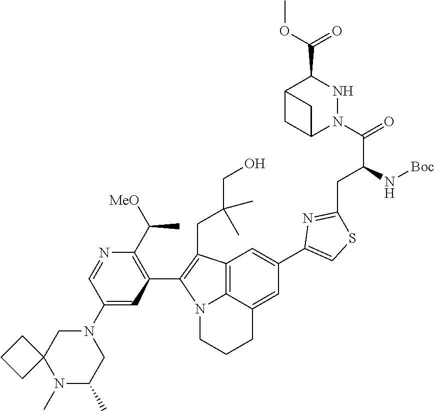

The title compound (510 mg, 80%) was prepared in a manner similar to that in example 16 step 3 from [(2M)-2-{5-[(6S)-5,6-dimethyl-5,8-diazaspiro[3.5]nonan-8-yl]-2-[(1S)-1-methoxyethyl]pyridin-3-yl}-3-(3-hydroxy-2,2-dimethylpropyl)-1-azatricyclo[6.3.1.0^{4,12}]dodeca-2,4(12),5,7-tetraen-6-yl]boronic acid and methyl (4S)-2-[(2S)-3-(4-bromo-1,3-thiazol-2-yl)-2-{[(tert-butoxy)carbonyl]amino}propanoyl]-2,3-diazabicyclo[3.1.1]heptane-4-carboxylate. LC-MS (M+H)⁺=939.3.

### Step 11: (4S)-2-[(2S)-2-{[(tert-butoxy)carbonyl]amino}-3-{4-[(2M)-2-{5-[(6S)-5,6-dimethyl-5,8-diazaspiro[3.5]nonan-8-yl]-2-[(1S)-1-methoxyethyl]pyridin-3-yl}-3-(3-hydroxy-2,2-dimethylpropyl)-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic Acid

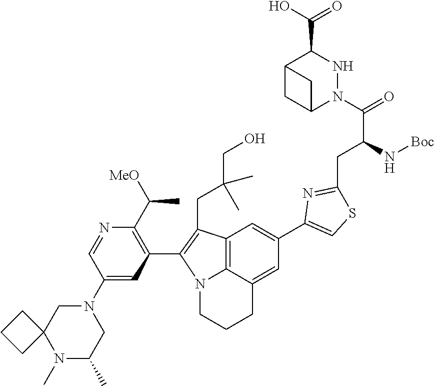

The title compound (430 mg, 86%) was prepared in a manner similar to that in example 16 step 4 from methyl (4S)-2-[(2S)-2-{[(tert-butoxy)carbonyl]amino}-3-{4-[(2M)-2-{5-[(6S)-5,6-dimethyl-5,8-diazaspiro[3.5]nonan-8-yl]-2-[(1S)-1-methoxyethyl]pyridin-3-yl}-3-(3-hydroxy-2,2-dimethylpropyl)-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylate. LC-MS (M+H)⁺=925.3.

### Step 12: tert-butyl N-[(7S,13S,19M)-20-{5-[(6S)-5,6-dimethyl-5,8-diazaspiro[3.5]nonan-8-yl]-2-[(1S)-1-methoxyethyl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate

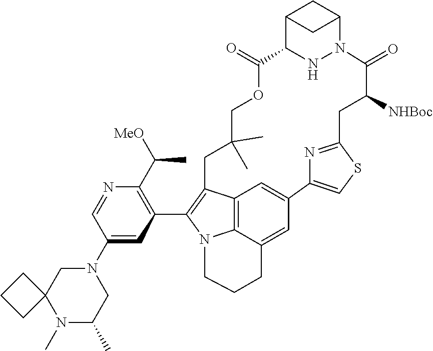

The title compound (260 mg, 62%) was prepared in a manner similar to that in example 16 step 5 from (4S)-2-[(2S)-2-{[(tert-butoxy)carbonyl]amino}-3-{4-[(2M)-2-{5-[(6S)-5,6-dimethyl-5,8-diazaspiro[3.5]nonan-8-yl]-2-[(1S)-1-methoxyethyl]pyridin-3-yl}-3-(3-hydroxy-2,2-dimethylpropyl)-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic acid. LC-MS (M+H)⁺=907.3.

### Step 13: (7S,13S,19M)-7-amino-20-{5-[(6S)-5,6-dimethyl-5,8-diazaspiro[3.5]nonan-8-yl]-2-[(1S)-1-methoxyethyl]pyridin-3-yl}-17,17-dimethyl-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione

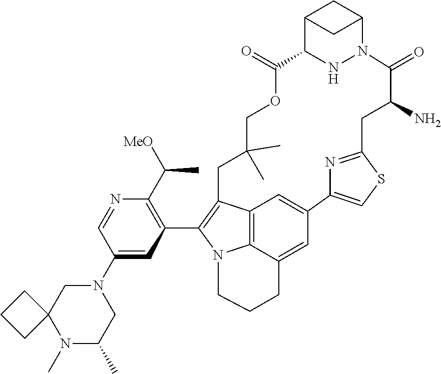

The title compound (230 mg, 99%) was prepared in a manner similar to that in example 16 step 6 from tert-butyl N-[(7S,13S,19M)-20-{5-[(6S)-5,6-dimethyl-5,8-diazaspiro[3.5]nonan-8-yl]-2-[(1S)-1-methoxyethyl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate. LC-MS (M+H)⁺=807.3.

### Step 14: (1R,2R,3S)—N-[(7S,13S,19M)-20-{5-[(6S)-5,6-dimethyl-5,8-diazaspiro[3.5]nonan-8-yl]-2-[(1S)-1-methoxyethyl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

Example 59 (8 mg, 6%) was prepared in a manner similar to that in example 16 step 7 from (7S,13S,19M)-7-amino-20-{5-[(6S)-5,6-dimethyl-5,8-diazaspiro[3.5]nonan-8-yl]-2-[(1S)-1-methoxyethyl]pyridin-3-yl}-17,17-dimethyl-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione and (1r,2R,3S)-2,3-dimethylcyclopropane-1-carboxylic acid. ¹H NMR (400 MHz, DMSO-d₆) δ 8.46 (d, J=2.8 Hz, 1H), 8.38 (d, J=8.9 Hz, 1H), 8.22 (s, 1H), 7.69 (s, 1H), 7.40 (s, 1H), 7.16 (d, J=2.9 Hz, 1H), 5.90 (d, J=11.0 Hz, 1H), 5.38-5.30 (m, 1H), 4.64 (d, J=10.9 Hz, 1H), 4.50-4.40 (m, 1H), 4.29-4.13 (m, 2H), 3.71-3.35 (m, 5H), 3.27-3.15 (m, 4H), 3.09 (dd, J=14.9, 7.3 Hz, 1H), 3.01-2.82 (m, 3H), 2.76-1.99 (m, 15H), 1.88-1.48 (m, 5H), 1.31 (d, J=6.0 Hz, 3H), 1.23-1.12 (m, 3H), 1.09-1.01 (m, 6H), 0.97 (d, J=6.1 Hz, 3H), 0.86 (s, 3H), 0.37 (s, 3H). LC-MS (M+H)⁺=903.3.

### Example 60: (1S,2S)—N-[(7S,13S,19M)-20-{5-[(6S)-5,6-dimethyl-5,8-diazaspiro[3.5]nonan-8-yl]-2-[(1S)-1-methoxyethyl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2-methylcyclopropane-1-carboxamide

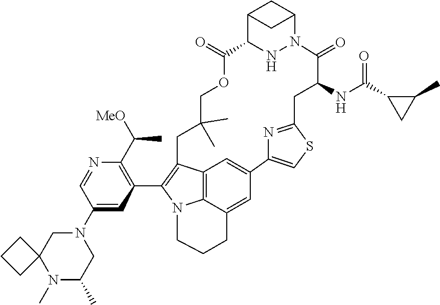

Example 60 (16 mg, 13%) was prepared in a manner similar to that in example 16 step 7 from (7S,13S,19M)-7-amino-20-{5-[(6S)-5,6-dimethyl-5,8-diazaspiro[3.5]nonan-8-yl]-2-[(1S)-1-methoxyethyl]pyridin-3-yl}-17,17-dimethyl-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione and (1S,2S)-2-methylcyclopropane-1-carboxylic acid. ¹H NMR (600 MHz, DMSO-d₆) δ 8.53 (d, J=8.9 Hz, 1H), 8.49 (d, J=2.9 Hz, 1H), 8.25 (s, 1H), 7.72 (s, 1H), 7.44 (s, 1H), 7.19 (d, J=2.9 Hz, 1H), 5.94 (d, J=11.0 Hz, 1H), 5.44-5.33 (m, 1H), 4.68 (d, J=11.1 Hz, 1H), 4.56-4.41 (m, 1H), 4.33-4.16 (m, 2H), 3.71-3.49 (m, 4H), 3.45-3.39 (m, 1H), 3.28-3.20 (m, 4H), 3.13 (dd, J=14.9, 7.4 Hz, 1H), 3.05-2.87 (m, 3H), 2.76 (d, J=12.0 Hz, 1H), 2.66-2.03 (m, 14H), 1.90-1.72 (m, 2H), 1.69-1.54 (m, 3H), 1.51-1.47 (m, 1H), 1.35 (d, J=6.1 Hz, 3H), 1.13-1.05 (m, 4H), 1.01 (d, J=6.3 Hz, 3H), 0.94-0.85 (m, 4H), 0.60-0.53 (m, 1H), 0.40 (s, 3H). LC-MS (M+H)⁺=889.3.

### Example 61: (1R,2R,3S)—N-[(7S,13S,19M)-20-{2-[(1S)-1-methoxyethyl]-5-{5-methyl-5,8-diazaspiro[3.5]nonan-8-yl}pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

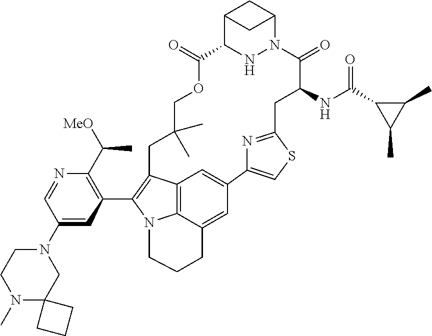

### Step 1: 3-[(2M)-6-bromo-2-{2-[(S)-1-methoxyethyl]-5-{5-methyl-5,8-diazaspiro[3.5]nonan-8-yl}pyridin-3-yl}-1-azatricyclo[6.3.1.0^{4,12}]dodeca-2,4(12),5,7-tetraen-3-yl]-2,2-dimethylpropan-1-ol

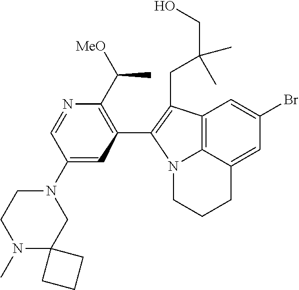

The title compound (870 mg, 57%) was prepared in a manner similar to that in example 20 step 1 from 3-[(2M)-6-bromo-2-{2-[(1S)-1-methoxyethyl]-5-(4,4,5,5-tetramethyl-1,3,2-dioxaborolan-2-yl)pyridin-3-yl}-1-azatricyclo[6.3.1.0^{4,12}]dodeca-2,4(12),5,7-tetraen-3-yl]-2,2-dimethylpropan-1-ol and 5-methyl-5,8-diazaspiro[3.5]nonane. LC-MS (M+H)⁺=595.2/597.2.

### Step 2: [(2M)-3-(3-hydroxy-2,2-dimethylpropyl)-2-{2-[(1S)-1-methoxyethyl]-5-{5-methyl-5,8 dizaspiro[3.5]nonan-8-yl}pyridin-3-yl}-1-azatricyclo[6.3.1.0^{4,12}]dodeca-2,4(12),5,7-tetraen-6-yl]boronic Acid

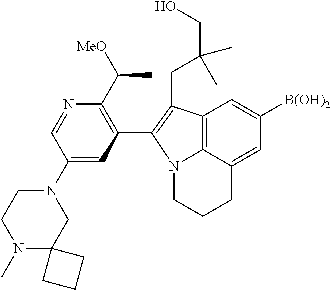

The title compound (240 mg, 94%) was prepared in a manner similar to that in example 35 step 3 from 3-[(2M)-6-bromo-2-{2-[(1S)-1-methoxyethyl]-5-{5-methyl-5,8-diazaspiro[3.5]nonan-8-yl}pyridin-3-yl}-1-azatricyclo[6.3.1.0^{4,12}]dodeca-2,4(12),5,7-tetraen-3-yl]-2,2-dimethylpropan-1-ol. LC-MS (M+H)⁺=561.3.

### Step 3: methyl (4S)-2-[(2S)-2-{[(1R,2R,3S)-2,3-dimethylcyclopropyl]formamido}-3-{4-[(2M)-3-(3-hydroxy-2,2-dimethylpropyl)-2-{2-[(1S)-1-methoxyethyl]-5-{5-methyl-5,8-diazaspiro[3.5]nonan-8-yl}pyridin-3-yl}-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylate

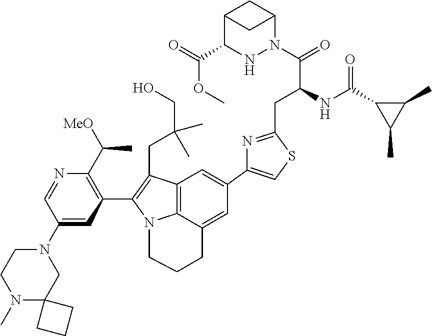

The title compound (290 mg, 70%) was prepared in a manner similar to that in example 35 step 4 from [(2M)-3-(3-hydroxy-2,2-dimethylpropyl)-2-{2-[(1S)-1-methoxyethyl]-5-{5-methyl-5,8-diazaspiro[3.5]nonan-8-yl}pyridin-3-yl}-1-azatricyclo[6.3.1.0^{4,12}]dodeca-2,4(12),5,7-tetraen-6-yl]boronic acid and methyl (4S)-2-[(2S)-3-(4-bromo-1,3-thiazol-2-yl)-2-{[(1R,2R,3S)-2,3-dimethylcyclopropyl]formamido}propanoyl]-2,3-diazabicyclo[3.1.1]heptane-4-carboxylate. LC-MS (M+H)⁺=921.5.

### Step 4: (4S)-2-[(2S)-2-{[(1R,2R,3S)-2,3-dimethylcyclopropyl]formamido}-3-{4-[(2M)-3-(3-hydroxy-2,2-dimethylpropyl)-2-{2-[(1S)-1-methoxyethyl]-5-{5-methyl-5,8-diazaspiro[3.5]nonan-8-yl}pyridin-3-yl}-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic Acid

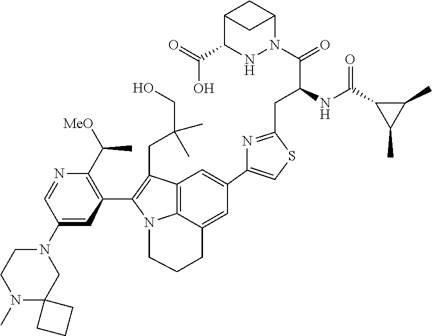

The title compound (240 mg, 82%) was prepared in a manner similar to that in example 35 step 5 from methyl (4S)-2-[(2S)-2-{[(R,2R,3S)-2,3-dimethylcyclopropyl]formamido}-3-{4-[(2M)-3-(3-hydroxy-2,2-dimethylpropyl)-2-{2-[(1S)-1-methoxyethyl]-5-{5-methyl-5,8-diazaspiro[3.5]nonan-8-yl}pyridin-3-yl}-1-azatricyclo[6.3.1.0^{4,12}]dodeca-2,4(12),5,7-tetraen-6-yl]-1,3-thiazol-2-yl)propanoyl]-2,3-diazabicyclo[3.1.1]heptane-4-carboxylate. LC-MS (M+H)⁺=907.4.

### Step 5: (1R,2R,3S)—N-[(7S,13S,19M)-20-{2-[(1S)-1-methoxyethyl]-5-{5-methyl-5,8-diazaspiro[3.5]nonan-8-yl}pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

Example 61 (30 mg, 12%) was prepared in a manner similar to that in example 35 step 6 from (4S)-2-[(2S)-2-{[(R,2R,3S)-2,3-dimethylcyclopropyl]formamido}-3-{4-[(2M)-3-(3-hydroxy-2,2-dimethylpropyl)-2-{2-[(1S)-1-methoxyethyl]-5-{5-methyl-5,8-diazaspiro[3.5]nonan-8-yl}pyridin-3-yl}-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic acid. ¹HNMR (400 MHz, DMSO-d₆) δ 8.44 (d, J=2.8 Hz, 1H), 8.36 (d, J=8.9 Hz, 1H), 8.22 (s, 1H), 7.68 (s, 1H), 7.40 (s, 1H), 7.17 (d, J=2.9 Hz, 1H), 5.89 (d, J=11.0 Hz, 1H), 5.61-5.15 (m, 1H), 4.63 (d, J=10.9 Hz, 1H), 4.48-4.39 (m, 1H), 4.32-4.08 (m, 2H), 3.71-3.46 (m, 3H), 3.24-3.06 (m, 9H), 3.03-2.82 (m, 3H), 2.65-2.57 (m, 1H), 2.51-2.05 (m, 13H), 1.90-1.74 (m, 1H), 1.66-1.47 (m, 4H), 1.31 (d, J=6.0 Hz, 3H), 1.27-1.11 (m, 3H), 1.08-0.99 (m, 6H), 0.85 (s, 3H), 0.37 (s, 3H). LC-MS (M+H)⁺=889.4.

### Example 62: (1S,2S)—N-[(7S,13S,19M)-20-{2-[(1S)-1-methoxyethyl]-5-{5-methyl-5,8-diazaspiro[3.5]nonan-8-yl}pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2-methylcyclopropane-1-carboxamide

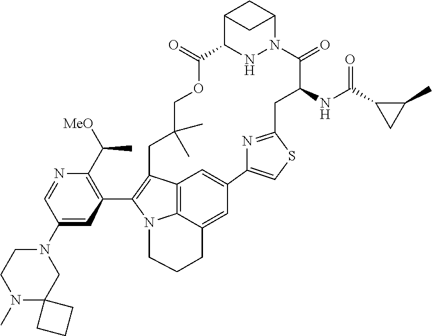

### Step 1: methyl (4S)-2-[(2S)-3-{4-[(2M)-3-(3-hydroxy-2,2-dimethylpropyl)-2-{2-[(1S)-1-methoxyethyl]-5-{5-methyl-5,8-diazaspiro[3.5]nonan-8-yl}pyridin-3-yl}-1-azatricyclo[6.3.1.0^{4,12}]dodeca-2,4(12),5,7-tetraen-6-yl]-1,3-thiazol-2-yl}-2-{[(1S,2S)-2-methylcyclopropyl]formamido}propanoyl]-2,3-diazabicyclo[3.1.1]heptane-4-carboxylate

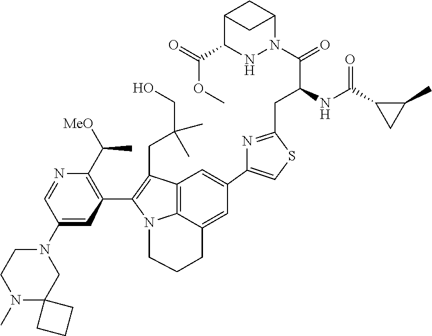

The title compound (270 mg, 65%) was prepared in a manner similar to that in example 35 step 4 from [(2M)-3-(3-hydroxy-2,2-dimethylpropyl)-2-{2-[(1S)-1-methoxyethyl]-5-{5-methyl-5,8-diazaspiro[3.5]nonan-8-yl}pyridin-3-yl}-1-azatricyclo[6.3.1.0^{4,12}]dodeca-2,4(12),5,7-tetraen-6-yl]boronic acid and methyl (4S)-2-[(2S)-3-(4-bromo-1,3-thiazol-2-yl)-2-{[(1S,2S)-2-methylcyclopropyl]formamido}propanoyl]-2,3-diazabicyclo[3.1.1]heptane-4-carboxylate. LC-MS (M+H)⁺=907.4.

### Step 2: (4S)-2-[(2S)-3-{4-[(2M)-3-(3-hydroxy-2,2-dimethylpropyl)-2-{2-[(1S)-1-methoxyethyl]-5-{5-methyl-5,8-diazaspiro[3.5]nonan-8-yl]pyridin-3-yl}-1-azatricyclo[6.3.1.0^{4,12}]dodeca-2,4(12),5,7-tetraen-6-yl]-1,3-thiazol-2-yl}-2-1[(1S,2S)-2-methylcyclopropyl]formamido}propanoyl]-2,3-diazabicyclo[3.1.1]heptane-4-carboxylic Acid

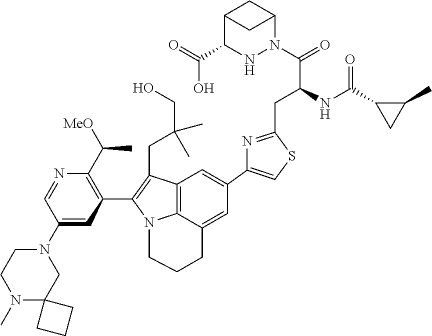

The title compound (234 mg, 86%) was prepared in a manner similar to that in example 35 step 5 from methyl (4S)-2-[(2S)-3-{4-[(2M)-3-(3-hydroxy-2,2-dimethylpropyl)-2-{2-[(1S)-1-methoxyethyl]-5-{5-methyl-5,8-diazaspiro[3.5]nonan-8-yl}pyridin-3-yl}-1-azatricyclo[6.3.1.0^{4,12}]dodeca-2,4(12),5,7-tetraen-6-yl]-1,3-thiazol-2-yl}-2-{[(1S,2S)-2-methylcyclopropyl]formamido}propanoyl]-2,3-diazabicyclo[3.1.1]heptane-4-carboxylate. LC-MS (M+H)⁺=893.4.

### Step 3: (1S,2S)—N-[(7S,13S,19M)-20-{2-[(1S)-1-methoxyethyl]-5-{5-methyl-5,8-diazaspiro[3.5]nonan-8-yl}pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2-methylcyclopropane-1-carboxamide

Example 62 (30 mg, 13%) was prepared in a manner similar to that in example 35 step 6 from (4S)-2-[(2S)-3-{4-[(2M)-3-(3-hydroxy-2,2-dimethylpropyl)-2-{2-[(1S)-1-methoxyethyl]-5-{5-methyl-5,8-diazaspiro[3.5]nonan-8-yl}pyridin-3-yl}-1-azatricyclo[6.3.1.0^{4,12}]dodeca-2,4(12),5,7-tetraen-6-yl]-1,3-thiazol-2-yl)-2-{[(S,2S)-2-methylcyclopropyl]formamido}propanoyl]-2,3-diazabicyclo[3.1.1]heptane-4-carboxylic acid. ¹H NMR (400 MHz, DMSO-d₆) δ 8.48 (d, J=9.2 Hz, 1H), 8.44 (s, 1H), 8.22 (s, 1H), 7.68 (s, 1H), 7.40 (s, 1H), 7.16 (s, 1H), 5.90 (d, J=11.0 Hz, 1H), 5.58-5.24 (m, 1H), 4.64 (d, J=10.9 Hz, 1H), 4.53-4.40 (m, 1H), 4.30-4.08 (m, 2H), 3.70-3.42 (m, 3H), 3.23-3.07 (m, 9H), 3.02-2.82 (m, 3H), 2.67-2.56 (m, 1H), 2.55-2.05 (m, 13H), 1.89-1.74 (m, 1H), 1.66-1.41 (m, 5H), 1.31 (d, J=5.9 Hz, 3H), 1.09-0.98 (m, 4H), 0.94-0.80 (m, 4H), 0.58-0.48 (m, 1H), 0.36 (s, 3H). LC-MS (M+H)⁺=875.4.

### Example 63: (1R,2R,3S)—N-[(7S,13S,19M)-20-(5-(5-ethyl-5,8-diazaspiro[3.5]nonan-8-yl)-2-[(1S)-1-methoxyethyl]pyridin-3-yl)-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

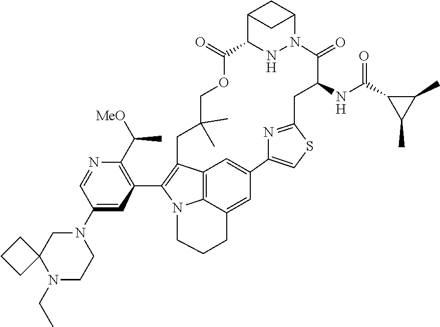

### Step 1: tert-butyl 8-[(5M)-5-[6-bromo-3-(3-hydroxy-2,2-dimethylpropyl)-1-azatricyclo[6.3.1.0^{4,12}]dodeca-2,4(12),5,7-tetraen-2-yl]-6-[(1S)-1-methoxyethyl]pyridin-3-yl]-5,8-diazaspiro[3.5]nonane-5-carboxylate

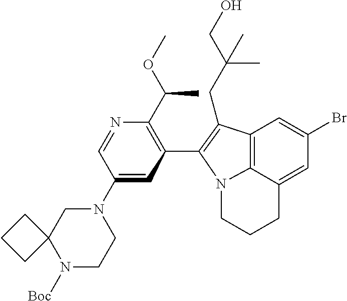

The title compound (1.5 g, 55%) was prepared in a manner similar to that in example 20 step 1 from 3-[(2M)-6-bromo-2-{2-[(1S)-1-methoxyethyl]-5-(4,4,5,5-tetramethyl-1,3,2-dioxaborolan-2-yl)pyridin-3-yl}-1-azatricyclo[6.3.1.0^{4,12}]dodeca-2,4(12),5,7-tetraen-3-yl]-2,2-dimethylpropan-1-ol and tert-butyl 5,8-diazaspiro[3.5]nonane-5-carboxylate. LC-MS (M+H)⁺=681.3/683.3.

### Step 2: 3-[(2M)-6-bromo-2-(5-{5,8-diazaspiro[3.5]nonan-8-yl}-2-[(1S)-1-methoxyethyl]pyridin-3-yl)-1-azatricyclo[6.3.1.0^{4,12}]dodeca-2,4(12),5,7-tetraen-3-yl]-2,2-dimethylpropan-1-ol

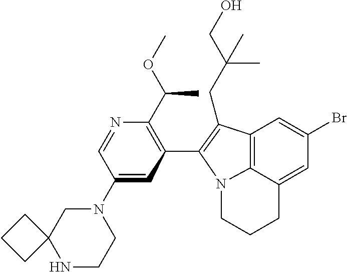

The title compound (1.3 g, 100%) was prepared in a manner similar to that in example 26 step 2 from tert-butyl 8-[(5M)-5-[6-bromo-3-(3-hydroxy-2,2-dimethylpropyl)-1-azatricyclo[6.3.1.0^{4,12}]dodeca-2,4(12),5,7-tetraen-2-yl]-6-[(1S)-1-methoxyethyl]pyridin-3-yl]-5,8-diazaspiro[3.5]nonane-5-carboxylate. LC-MS (M+H)⁺=581.3/583.3.

### Step 3: 3-[(2M)-6-bromo-2-(5-{5-ethyl-5,8-diazaspiro[3.5]nonan-8-yl}-2-[(1S)-1-methoxyethyl]pyridin-3-yl)-1-azatricyclo[6.3.1.0^{4,12}]dodeca-2,4(12),5,7-tetraen-3-yl]-2,2-dimethylpropan-1-ol

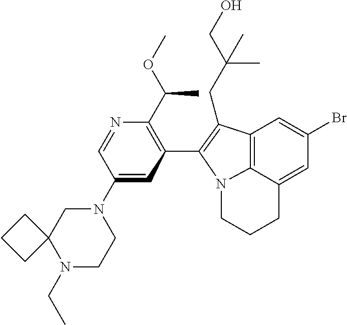

The title compound (0.7 g, 51%) was prepared in a manner similar to that in example 51 step 3 from 3-[(2M)-6-bromo-2-(5-{5,8-diazaspiro[3.5]nonan-8-yl}-2-[(1S)-1-methoxyethyl]pyridin-3-yl)-1-azatricyclo[6.3.1.0^{4,12}]dodeca-2,4(12),5,7-tetraen-3-yl]-2,2-dimethylpropan-1-ol. LC-MS (M+H)⁺=609.3/611.3.

### Step 4: [(2M)-2-(5-{5-ethyl-5,8-diazaspiro[3.5]nonan-8-yl}-2-[(1S)-1-methoxyethyl]pyridin-3-yl)-3-(3-hydroxy-2,2-dimethylpropyl)-1-azatricyclo[6.3.1.0^{4,12}]dodeca-2,4(12),5,7-tetraen-6-yl]boronic Acid

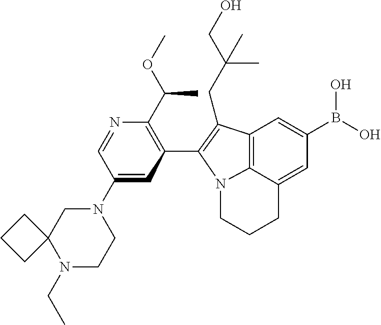

The title compound (0.7 g, 100%) was prepared in a manner similar to that in example 35 step 3 from 3-[(2M)-6-bromo-2-(5-{5-ethyl-5,8-diazaspiro[3.5]nonan-8-yl}-2-[(1S)-1-methoxyethyl]pyridin-3-yl)-1-azatricyclo[6.3.1.0^{4,12}]dodeca-2,4(12),5,7-tetraen-3-yl]-2,2-dimethylpropan-1-ol. LC-MS (M+H)⁺=575.3.

### Step 5: methyl (4S)-2-[(2S)-2-{[(1R,2R,3S)-2,3-dimethylcyclopropyl]formamido}-3-{4-[(2M)-2-(5-{5-ethyl-5,8-diazaspiro[3.5]nonan-8-yl}-2-[(1S)-1-methoxyethyl]pyridin-3-yl)-3-(3-hydroxy-2,2-dimethylpropyl)-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylate

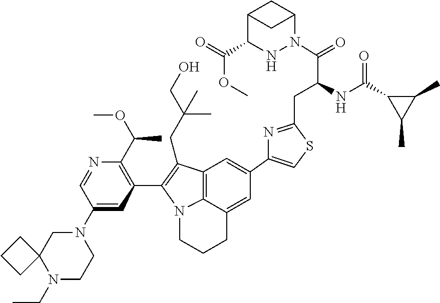

The title compound (0.43 g, 74%) was prepared in a manner similar to that in example 35 step 4 from [(2M)-2-(5-{5-ethyl-5,8-diazaspiro[3.5]nonan-8-yl}-2-[(1S)-1-methoxyethyl]pyridin-3-yl)-3-(3-hydroxy-2,2-dimethylpropyl)-1-azatricyclo[6.3.1.0^{4,12}]dodeca-2,4(12),5,7-tetraen-6-yl]boronic acid and methyl (4S)-2-[(2S)-3-(4-bromo-1,3-thiazol-2-yl)-2-{[(1R,2R,3S)-2,3-dimethylcyclopropyl]formamido}propanoyl]-2,3-diazabicyclo[3.1.1]heptane-4-carboxylate. LC-MS (M+H)⁺=935.3.

### Step 6: (4S)-2-[(2S)-2-{[(1R,2R,3S)-2,3-dimethylcyclopropyl]formamido}-3-{4-[(2M)-2-(5-{5-ethyl-5,8-diazaspiro[3.5]nonan-8-yl}-2-[(1S)-1-methoxyethyl]pyridin-3-yl)-3-(3-hydroxy-2,2-dimethylpropyl)-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic Acid

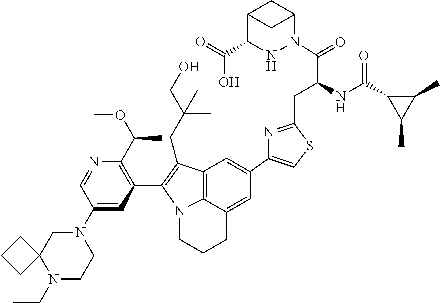

The title compound (0.15 g, 35%) was prepared in a manner similar to that in example 35 step 5 from methyl (4S)-2-[(2S)-2-{[(R,2R,3S)-2,3-dimethylcyclopropyl]formamido}-3-{4-[(2M)-2-(5-{5-ethyl-5,8-diazaspiro[3.5]nonan-8-yl}-2-[(1S)-1-methoxyethyl]pyridin-3-yl)-3-(3-hydroxy-2,2-dimethylpropyl)-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylate. LC-MS (M+H)⁺=921.3.

### Step 7: (1R,2R,3S)—N-[(7S,13S,19M)-20-(5-{5-ethyl-5,8-diazaspiro[3.5]nonan-8-yl}-2-[(1S)-1-methoxyethyl]pyridin-3-yl]-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

The title compound (35 mg, 24%) was prepared in a manner similar to that in example 35 step 6 from (4S)-2-[(2S)-2-{[(R,2R,3S)-2,3-dimethylcyclopropyl]formamido}-3-{4-[(2M)-2-(5-(5-ethyl-5,8-diazaspiro[3.5]nonan-8-yl}-2-[(1S)-1-methoxyethyl]pyridin-3-yl)-3-(3-hydroxy-2,2-dimethylpropyl)-1-azatricyclo[6.3.1.0^{4,12}]dodeca-2,4(12),5,7-tetraen-6-yl]-1,3-thiazol-2-yl)propanoyl]-2,3-diazabicyclo[3.1.1]heptane-4-carboxylic acid. ¹H NMR (400 MHz, DMSO-d₆) δ 8.44 (d, J=2.9 Hz, 1H), 8.39 (d, J=8.9 Hz, 1H), 8.22 (s, 1H), 7.69 (s, 1H), 7.41 (s, 1H), 7.16 (d, J=2.9 Hz, 1H), 5.90 (d, J=11.0 Hz, 1H), 5.43-5.24 (m, 1H), 4.64 (d, J=10.8 Hz, 1H), 4.53-4.39 (m, 1H), 4.36-4.11 (m, 2H), 3.65-3.43 (m, 3H), 3.24-3.05 (m, 9H), 3.02-2.82 (m, 3H), 2.64-2.01 (m, 13H), 1.86-1.74 (m, 1H), 1.72-1.59 (m, 3H), 1.52 (t, J=9.2 Hz, 1H), 1.31 (d, J=6.0 Hz, 3H), 1.24-1.10 (m, 3H), 1.09-1.01 (m, 6H), 0.98 (t, J=7.1 Hz, 3H), 0.85 (s, 3H), 0.36 (s, 3H). LC-MS (M+H)⁺=903.3.

### Example 64: (1S,2S)—N-[(7S,13S,19M)-20-(5-(5-ethyl-5,8-diazaspiro[3.5]nonan-8-yl)-2-[(1S)-1-methoxyethyl]pyridin-3-yl)-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2-methylcyclopropane-1-carboxamide

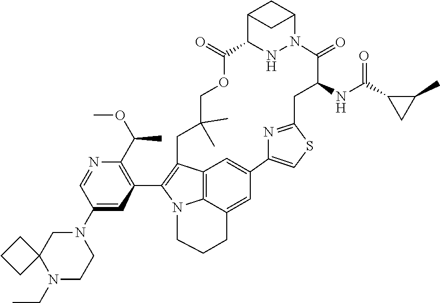

### Step 1: methyl (4S)-2-[(2S)-3-{4-[(2M)-2-(5-{5-ethyl-5,8-diazaspiro[3.5]nonan-8-yl}-2-[(1S)-1-methoxyethyl]pyridin-3-yl)-3-(3-hydroxy-2,2-dimethylpropyl)-1-azatricyclo[6.3.1.0^{4,12}]dodeca-2,4(12),5,7-tetraen-6-yl]-1,3-thiazol-2-yl}-2-{[(1S,2S)-2-methylcyclopropyl]formamido}propanoyl]-2,3-diazabicyclo[3.1.1]heptane-4-carboxylate

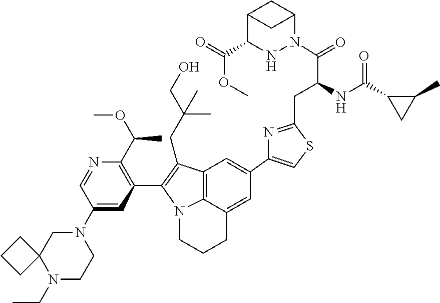

The title compound (0.22 g, 39%) was prepared in a manner similar to that in example 35 step 4 from [(2M)-2-(5-{5-ethyl-5,8-diazaspiro[3.5]nonan-8-yl}-2-[(1S)-1-methoxyethyl]pyridin-3-yl)-3-(3-hydroxy-2,2-dimethylpropyl)-1-azatricyclo[6.3.1.0^{4,12}]dodeca-2,4(12),5,7-tetraen-6-yl]boronic acid and methyl (4S)-2-[(2S)-3-(4-bromo-1,3-thiazol-2-yl)-2-{[(S,2S)-2-methylcyclopropyl]formamido}propanoyl]-2,3-diazabicyclo[3.1.1]heptane-4-carboxylate. LC-MS (M+H)⁺=921.3.

### Step 2: (4S)-2-[(2S)-3-{4-[(2M)-2-(5-{5-ethyl-5,8-diazaspiro[3.5]nonan-8-yl}-2-[(1S)-1-methoxyethyl]pyridin-3-yl)-3-(3-hydroxy-2,2-dimethylpropyl)-1-azatricyclo[6.3.1.0^{4,12}]dodeca-2,4(12),5,7-tetraen-6-yl]-1,3-thiazol-2-yl}-2-{[(1S,2S)-2-methylcyclopropyl]formamido}propanoyl]-2,3-diazabicyclo[3.1.1]heptane-4-carboxylic Acid

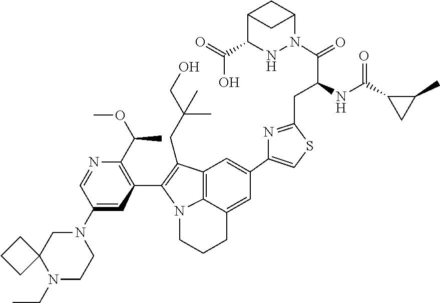

The title compound (135 mg, 62%) was prepared in a manner similar to that in example 35 step 5 from methyl (4S)-2-[(2S)-3-{4-[(2M)-2-(5-{5-ethyl-5,8-diazaspiro[3.5]nonan-8-yl}-2-[(1S)-1-methoxyethyl]pyridin-3-yl)-3-(3-hydroxy-2,2-dimethylpropyl)-1-azatricyclo[6.3.1.0^{4,12}]dodeca-2,4(12),5,7-tetraen-6-yl]-1,3-thiazol-2-yl}-2-{[(1S,2S)-2-methylcyclopropyl]formamido}propanoyl]-2,3-diazabicyclo[3.1.1]heptane-4-carboxylate. LC-MS (M+H)⁺=907.3.

### Step 3: (1S,2S)—N-[(7S,13S,19M)-20-(5-{5-ethyl-5,8-diazaspiro[3.5]nonan-8-yl}-2-[(1S)-1-methoxyethyl]pyridin-3-yl)-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2-methylcyclopropane-1-carboxamide

Example 64 (45 mg, 34%) was prepared in a manner similar to that in example 35 step 6 from (4S)-2-[(2S)-3-{4-[(2M)-2-(5-{5-ethyl-5,8-diazaspiro[3.5]nonan-8-yl}-2-[(1S)-1-methoxyethyl]pyridin-3-yl)-3-(3-hydroxy-2,2-dimethylpropyl)-1-azatricyclo[6.3.1.0^{4,12}]dodeca-2,4(12),5,7-tetraen-6-yl]-1,3-thiazol-2-yl}-2-{[(S,2S)-2-methylcyclopropyl]formamido}propanoyl]-2,3-diazabicyclo[3.1.1]heptane-4-carboxylic acid. ¹H NMR (400 MHz, DMSO-d₆) δ 8.50 (d, J=8.9 Hz, 1H), 8.44 (d, J=2.8 Hz, 1H), 8.21 (s, 1H), 7.68 (s, 1H), 7.41 (s, 1H), 7.16 (d, J=3.0 Hz, 1H), 5.91 (d, J=11.0 Hz, 1H), 5.54-5.21 (m, 1H), 4.65 (d, J=10.9 Hz, 1H), 4.50-4.38 (m, 1H), 4.31-4.08 (m, 2H), 3.66-3.41 (m, 3H), 3.25-3.05 (m, 9H), 3.04-2.81 (m, 3H), 2.64-2.02 (m, 13H), 1.86-1.59 (m, 4H), 1.55-1.42 (m, 2H), 1.31 (d, J=6.0 Hz, 3H), 1.05-0.93 (m, 7H), 0.88-0.81 (m, 4H), 0.59-0.44 (m, 1H), 0.36 (s, 3H). LC-MS (M+H)⁺=889.3.

### Example 65: (1R,2R,3S)—N-[(7S,13S,19M)-20-{5-[(6R)-5,6-dimethyl-5,8-diazaspiro[3.5]nonan-8-yl]-2-[(1S)-1-methoxyethyl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

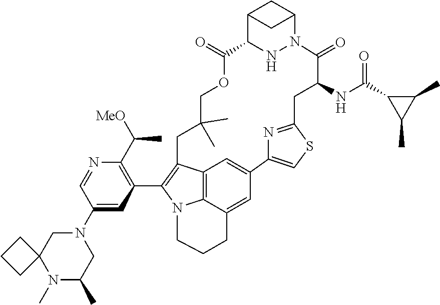

### Step 1: tert-butyl N-(1-{[(2S)-2-chloro-N-[(4-methoxyphenyl)methyl]propanamido]methyl}cyclobutyl)carbamate

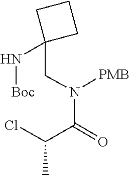

The title compound (10.5 g, 61%) was prepared in a manner similar to that in example 59 step 2 from tert-butyl N-[1-({[(4-methoxyphenyl)methyl]amino}methyl)cyclobutyl]carbamate and (2S)-2-chloropropanoic acid. LC-MS (M+H)⁺=411.3.

### Step 2: (2S)—N-[(1-aminocyclobutyl)methyl]-2-chloro-N-[(4-methoxyphenyl)methyl]propanamide hydrochloride

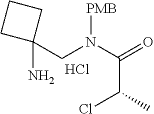

The title compound (7.1 g, 88%) was prepared in a manner similar to that in example 59 step 3 from tert-butyl N-(1-{[(2S)-2-chloro-N-[(4-methoxyphenyl)methyl]propanamido]methyl}cyclobutyl)carbamate. LC-MS (M+H)⁺=311.3.

### Step 3: (6R)-8-[(4-methoxyphenyl)methyl]-6-methyl-5,8-diazaspiro[3.5]nonan-7-one

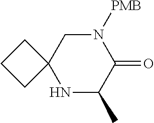

The title compound (5.0 g, 83%) was prepared in a manner similar to that in example 59 step 4 from (2S)—N-[(1-aminocyclobutyl)methyl]-2-chloro-N-[(4-methoxyphenyl)methyl]propanamide hydrochloride. LC-MS (M+H)⁺=275.3.

### Step 4: (6R)-8-[(4-methoxyphenyl)methyl]-6-methyl-5,8-diazaspiro[3.5]nonane

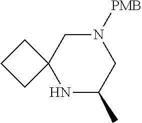

The title compound (4.1 g, 84%) was prepared in a manner similar to that in example 59 step 5 from (6R)-8-[(4-methoxyphenyl)methyl]-6-methyl-5,8-diazaspiro[3.5]nonan-7-one. LC-MS (M+H)⁺=261.3.

### Step 5: (6R)-8-[(4-methoxyphenyl)methyl]-5,6-dimethyl-5,8-diazaspiro[3.5]nonane

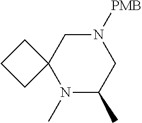

The title compound (2.1 g, 48%) was prepared in a manner similar to that in example 59 step 6 from (6R)-8-[(4-methoxyphenyl)methyl]-6-methyl-5,8-diazaspiro[3.5]nonane. LC-MS (M+H)⁺=275.3.

### Step 6: (6R)-5,6-dimethyl-5,8-diazaspiro[3.5]nonane

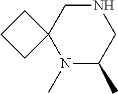

The title compound (0.75 g, 67%) was prepared in a manner similar to that in example 59 step 7 from (6R)-8-[(4-methoxyphenyl)methyl]-5,6-dimethyl-5,8-diazaspiro[3.5]nonane. LC-MS (M+H)+=155.3.

### Step 7: 3-[(2M)-6-bromo-2-{5-[(6R)-5,6-dimethyl-5,8-diazaspiro[3.5]nonan-8-yl]-2-[(1S)-1-methoxyethyl]pyridin-3-yl}-1-azatricyclo[6.3.1.0^{4,12}]dodeca-2,4(12),5,7-tetraen-3-yl]-2,2-dimethylpropan-1-ol

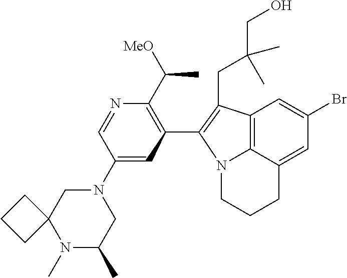

The title compound (130 mg, 22%) was prepared in a manner similar to that in example 20 step 1 from 3-[(2M)-6-bromo-2-{2-[(1S)-1-methoxyethyl]-5-(4,4,5,5-tetramethyl-1,3,2-dioxaborolan-2-yl)pyridin-3-yl}-1-azatricyclo[6.3.1.0^{4,12}]dodeca-2,4(12),5,7-tetraen-3-yl]-2,2-dimethylpropan-1-ol and (6R)-5,6-dimethyl-5,8-diazaspiro[3.5]nonane. LC-MS (M+H)⁺=609.4/611.3.

### Step 8: [(2M)-2-{5-[(6R)-5,6-dimethyl-5,8-diazaspiro[3.5]nonan-8-yl]-2-[(1S)-1-methoxyethyl]pyridin-3-yl}-3-(3-hydroxy-2,2-dimethylpropyl)-1-azatricyclo[6.3.1.0^{4,12}]dodeca-2,4(12),5,7-tetraen-6-yl]boronic Acid

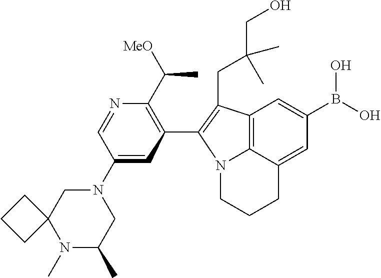

The title compound (120 mg, 99%) was prepared in a manner similar to that in example 35 step 3 from 3-[(2M)-6-bromo-2-{5-[(6R)-5,6-dimethyl-5,8-diazaspiro[3.5]nonan-8-yl]-2-[(1S)-1-methoxyethyl]pyridin-3-yl}-1-azatricyclo[6.3.1.0^{4,12}]dodeca-2,4(12),5,7-tetraen-3-yl]-2,2-dimethylpropan-1-ol. LC-MS (M+H)⁺=575.3.

### Step 9: methyl (4S)-2-[(2S)-2-{[(tert-butoxy)carbonyl]amino}-3-{4-[(2M)-2-{5-[(6R)-5,6-dimethyl-5,8-diazaspiro[3.5]nonan-8-yl]-2-[(1S)-1-methoxyethyl]pyridin-3-yl}-3-(3-hydroxy-2,2-dimethylpropyl)-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylate

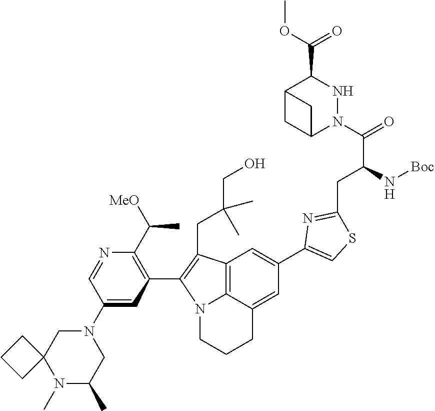

The title compound (160 mg, 82%) was prepared in a manner similar to that in example 16 step 3 from [(2M)-2-{5-[(6R)-5,6-dimethyl-5,8-diazaspiro[3.5]nonan-8-yl]-2-[(1S)-1-methoxyethyl]pyridin-3-yl}-3-(3-hydroxy-2,2-dimethylpropyl)-1-azatricyclo[6.3.1.0^{4,12}]dodeca-2,4(12),5,7-tetraen-6-yl]boronic acid and methyl (4S)-2-[(2S)-3-(4-bromo-1,3-thiazol-2-yl)-2-{[(tert-butoxy)carbonyl]amino}propanoyl]-2,3-diazabicyclo[3.1.1]heptane-4-carboxylate. LC-MS (M+H)⁺=939.3.

### Step 10: (4S)-2-[(2S)-2-{[(tert-butoxy)carbonyl]amino}-3-{4-[(2M)-2-{5-[(6R)-5,6-dimethyl-5,8-diazaspiro[3.5]nonan-8-yl]-2-[(1S)-1-methoxyethyl]pyridin-3-yl}-3-(3-hydroxy-2,2-dimethylpropyl)-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic Acid

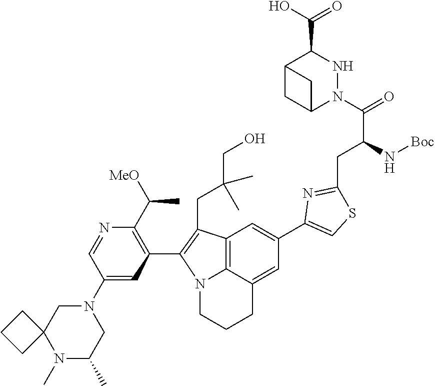

The title compound (130 mg, 83%) was prepared in a manner similar to that in example 16 step 4 from methyl (4S)-2-[(2S)-2-{[(tert-butoxy)carbonyl]amino}-3-{4-[(2M)-2-{5-[(6R)-5,6-dimethyl-5,8-diazaspiro[3.5]nonan-8-yl]-2-[(1S)-1-methoxyethyl]pyridin-3-yl}-3-(3-hydroxy-2,2-dimethylpropyl)-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylate. LC-MS (M+H)⁺=925.3.

### Step 11: tert-butyl N-[(7S,13S,19M)-20-{5-[(6R)-5,6-dimethyl-5,8-diazaspiro[3.5]nonan-8-yl]-2-[(1S)-1-methoxyethyl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate

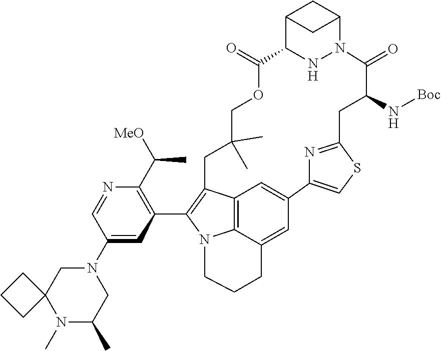

The title compound (80 mg, 63%) was prepared in a manner similar to that in example 16 step 5 from (4S)-2-[(2S)-2-{[(tert-butoxy)carbonyl]amino}-3-{4-[(2M)-2-{5-[(6R)-5,6-dimethyl-5,8-diazaspiro[3.5]nonan-8-yl]-2-[(1S)-1-methoxyethyl]pyridin-3-yl}-3-(3-hydroxy-2,2-dimethylpropyl)-1-azatricyclo[6.3.1.0^{4,12}]dodeca-2,4(12),5,7-tetraen-6-yl]-1,3-thiazol-2-yl}propanoyl]-2,3-diazabicyclo[3.1.1]heptane-4-carboxylic acid. LC-MS (M+H)⁺=907.3.

### Step 12: (7S,13S,19M)-7-amino-20-{5-[(6R)-5,6-dimethyl-5,8-diazaspiro[3.5]nonan-8-yl]-2-[(1S)-1-methoxyethyl]pyridin-3-yl}-17,17-dimethyl-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione

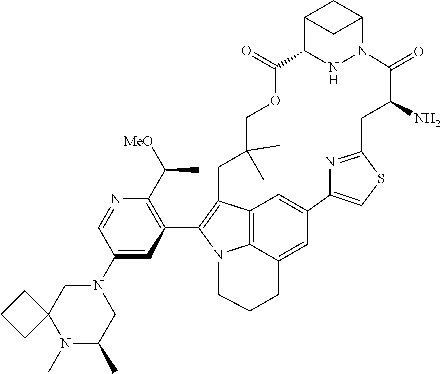

The title compound (70 mg, 99%) was prepared in a manner similar to that in example 16 step 6 from tert-butyl N-[(7S,13S,19M)-20-{5-[(6R)-5,6-dimethyl-5,8-diazaspiro[3.5]nonan-8-yl]-2-[(1S)-1-methoxyethyl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate. LC-MS (M+H)⁺=807.3.

### Step 13: (1R,2R,3S)—N-[(7S,13S,19M)-20-{5-[(6R)-5,6-dimethyl-5,8-diazaspiro[3.5]nonan-8-yl]-2-[(1S)-1-methoxyethyl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethylcyclopropane-1-carboxamide

Example 65 (23 mg, 70%) was prepared in a manner similar to that in example 16 step 7 from (7S,13S,19M)-7-amino-20-{5-[(6R)-5,6-dimethyl-5,8-diazaspiro[3.5]nonan-8-yl]-2-[(1S)-1-methoxyethyl]pyridin-3-yl}-17,17-dimethyl-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione and (1r,2R,3S)-2,3-dimethylcyclopropane-1-carboxylic acid. ¹H NMR (400 MHz, DMSO-d₆) δ 8.43 (d, J=2.9 Hz, 1H), 8.40 (d, J=9.3 Hz, 1H), 8.22 (s, 1H), 7.69 (s, 1H), 7.41 (s, 1H), 7.17 (d, J=2.9 Hz, 1H), 5.91 (d, J=11.0 Hz, 1H), 5.42-5.16 (m, 1H), 4.64 (d, J=11.0 Hz, 1H), 4.54-4.40 (m, 1H), 4.33-4.08 (m, 2H), 3.73 (d, J=12.3 Hz, 1H), 3.64-3.40 (m, 4H), 3.24-3.15 (m, 4H), 3.09 (dd, J=14.7, 7.3 Hz, 1H), 3.02-2.81 (m, 3H), 2.75 (d, J=12.2 Hz, 1H), 2.66-1.98 (m, 14H), 1.87-1.76 (m, 1H), 1.71-1.46 (m, 4H), 1.30 (d, J=5.8 Hz, 3H), 1.28-1.10 (m, 3H), 1.08-0.95 (m, 9H), 0.84 (s, 3H), 0.36 (s, 3H). LC-MS (M+H)⁺=903.3.

### Example 66: (1S,2S)—N-[(7S,13S,19M)-20-{5-[(6R)-5,6-dimethyl-5,8-diazaspiro[3.5]nonan-8-yl]-2-[(1S)-1-methoxyethyl]pyridin-3-yl}-17,17-dimethyl-8,14-dioxo-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2-methylcyclopropane-1-carboxamide

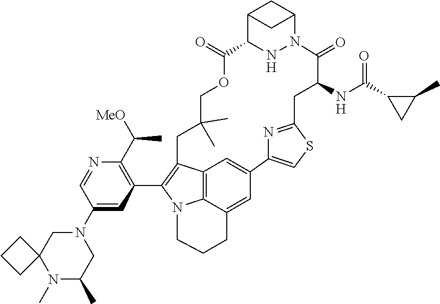

Example 66 (13 mg, 40%) was prepared in a manner similar to that in example 16 step 7 from (7S,13S,19M)-7-amino-20-{5-[(6R)-5,6-dimethyl-5,8-diazaspiro[3.5]nonan-8-yl]-2-[(1S)-1-methoxyethyl]pyridin-3-yl}-17,17-dimethyl-15-oxa-4-thia-9,21,30,32-tetraazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione and (1S,2S)-2-methylcyclopropane-1-carboxylic acid. ¹H NMR (400 MHz, DMSO-d₆) δ 8.48 (d, J=8.8 Hz, 1H), 8.42 (s, 1H), 8.22 (s, 1H), 7.67 (d, J=2.0 Hz, 1H), 7.40 (s, 1H), 7.16 (s, 1H), 5.90 (d, J=11.0 Hz, 1H), 5.37 (t, J=8.3 Hz, 1H), 4.76-4.55 (m, 1H), 4.47 (d, J=5.0 Hz, 1H), 4.32-4.03 (m, 2H), 3.72 (d, J=12.3 Hz, 1H), 3.63-3.40 (m, 4H), 3.26-3.15 (m, 4H), 3.09 (dd, J=14.9, 7.2 Hz, 1H), 3.05-2.83 (m, 3H), 2.76 (d, J=12.1 Hz, 1H), 2.63-2.03 (m, 14H), 1.83-1.75 (m, 1H), 1.72-1.43 (m, 5H), 1.31 (d, J=6.0 Hz, 3H), 1.08-0.95 (m, 7H), 0.86 (d, J=10.5 Hz, 4H), 0.56-0.48 (m, 1H), 0.36 (s, 3H). LC-MS (M+H)⁺=889.3.

### TEST EXAMPLES

Method 1: pERK Inhibition in AsPC-1 (KRAS G12D) Cells.

AsPC-1 cell line (pancreatic cancer cell line harboring KRAS G12D mutation) was used in this study. Cells were maintained in RPMI 1640 supplemented with 10% fetal bovine serum (Thermo Fisher), 50 units/mL penicillin and streptomycin (Thermo Fisher) and kept at 37° C. in a humidified atmosphere of 5% CO₂ in the air. Cells were reinstated from frozen stocks that were laid down within 30 passages from the original cells purchased. 30,000 cells per well were seeded into a 96-well plate and incubated overnight. Cells were treated with a 10-point dilution series of test compounds, with final compound concentration of from 0 to 10 μM. After 2 h compound treatment, cells were lysed, and the pERK1/2 (THR202/TYR204) level in the cell lysates was detected by HTRF kit (Cisbio) with protocol vide infra. In brief, a total of 16 μL of cell lysate from each well of a 96-well plate was transferred to a 384-well white assay plate. Lysate from each well was incubated with 2 μL of Eu³⁺-cryptate (donor) labeled anti-pERK1/2 and 2 μL of D2 (acceptor) labeled anti-pERK1/2 antibodies (Cisbio) overnight in dark at room temperature. When donor and acceptor are in proximity, excitation of the donor with laser triggers fluorescence resonance energy transfer towards the acceptor, which in turn fluoresces at 655 nm wavelength. FRET signals were measured using a PHERAstar FSX reader (BMG Labtech). IC50 determination was performed by fitting the curve of percent inhibition versus the log of the compounds concentration disclosed herein using Dotmatics.

The results of Method 1 were reported in Table 2.

TABLE 2
pERK inhibition assay in AsPC-1 cells

| Example | pERK IC₅₀ (nM) in AsPC-1 cells |
|---|---|
| 1 | A |
| 2 | B |
| 3 | B |
| 4 | A |
| 5 | B |
| 6 | B |
| 7 | A |
| 8 | A |
| 9 | A |
| 10 | B |
| 11 | A |
| 12 | B |
| 15 | A |
| 16 | A |
| 17 | B |
| 19 | B |
| 20 | A |
| 21 | A |
| 22 | A |
| 23 | A |
| 24 | A |
| 25 | A |
| 26 | A |
| 27 | A |
| 28 | B |
| 29 | B |
| 30 | B |
| 31 | B |
| 32 | A |
| 33 | B |
| 34 | A |
| 35 | A |
| 36 | A |
| 37 | A |
| 38 | A |
| 39 | A |
| 40 | A |
| 41 | A |
| 42 | A |
| 43 | A |
| 44 | A |
| 45 | A |
| 46 | A |
| 47 | A |
| 48 | A |
| 49 | A |
| 50 | A |
| 51 | A |
| 52 | B |
| 53 | B |
| 54 | B |
| 55 | A |
| 56 | B |
| 57 | A |
| 58 | B |
| 59 | A |
| 60 | B |
| 61 | A |
| 62 | B |
| 63 | A |
| 64 | B |
| 65 | A |
| 66 | B |

The activity in Table 2 is categorised as “A”, “B”, and “C” based on the corresponding value according to the following rules.

| Categorised | pERK IC₅₀ (nM) in AsPC-1 cells |
|---|---|
| A | less than 1.0 |
| B | no less than 1.0, and less than 10 |
| C | no less than 10, and less than 100 |
| D | no less than 100, and less than 10000 |

Method 2: pERK Inhibition in SW620 (KRAS G12V) Cells

SW620 cell line (colorectal colon cancer cell line harboring KRAS G12V mutation) was used in this study. Cells were maintained in RPMI 1640 supplemented with 10% fetal bovine serum (Thermo Fisher), 50 units/mL penicillin and streptomycin (Thermo Fisher) and kept at 37° C. in a humidified atmosphere of 5% CO₂ in the air. Cells were reinstated from frozen stocks that were laid down within 30 passages from the original cells purchased. 40000 cells per well were seeded into a 96-well plate and incubated overnight. Cells were treated with a 10-point dilution series of compounds disclosed herein with final compound concentration is from 0 to 10 μM. After 2 h compound treatment, cells were lysed, and the pERK1/2 (THR202/TYR204) level in the cell lysates was detected by HTRF kit (Cisbio) with protocol vide infra. In brief, a total of 16 μL of cell lysate from each well of a 96-well plate was transferred to a 384-well white assay plate. Lysate from each well was incubated with 2 μL of Eu³⁺-cryptate (donor) labeled anti-phospho-ERK1/2 and 2 μL of D2 (acceptor) labeled anti-pERK1/2 antibodies (Cisbio) overnight in dark at room temperature. When donor and acceptor are in proximity, excitation of the donor with laser triggers fluorescence resonance energy transfer towards the acceptor, which in turn fluoresces at 655 nm wavelength. FRET signals were measured using a PHERAstar FSX reader (BMG Labtech). IC50 determination was performed by fitting the curve of percent inhibition versus the log of the compounds concentration disclosed herein using Dotmatics.

The results of Method 2 were reported in Table 3.

TABLE 3
pERK inhibition assay in SW620 cells

| Example | pERK IC₅₀ (nM) in SW620 cells |
|---|---|
| 1 | A |
| 2 | A |
| 3 | A |
| 4 | A |
| 5 | A |
| 6 | A |
| 7 | A |
| 8 | A |
| 9 | A |
| 10 | A |
| 11 | A |
| 12 | A |
| 15 | A |
| 16 | A |
| 17 | A |
| 19 | A |
| 20 | A |
| 21 | A |
| 22 | A |
| 23 | A |
| 24 | A |
| 25 | A |
| 26 | A |
| 27 | A |
| 28 | A |
| 29 | A |
| 30 | A |
| 31 | A |
| 32 | A |
| 33 | A |
| 34 | A |
| 35 | A |
| 36 | A |
| 37 | A |
| 38 | A |
| 39 | A |
| 40 | A |
| 41 | A |
| 42 | A |
| 43 | A |
| 44 | A |
| 45 | A |
| 46 | A |
| 47 | A |
| 48 | A |
| 49 | A |
| 50 | A |
| 51 | A |
| 52 | A |
| 53 | A |
| 54 | A |
| 55 | A |
| 56 | A |
| 57 | A |
| 58 | A |
| 59 | A |
| 60 | A |
| 61 | A |
| 62 | A |
| 63 | A |
| 64 | A |
| 65 | A |
| 66 | A |

The activity in Table 3 is categorised as “A”, “B”, and “C” based on the corresponding value according to the following rules.

| Categorised | pERK IC₅₀ (nM) in SW620 cells |
|---|---|
| A | less than 1.0 |
| B | no less than 1.0, and less than 10 |
| C | no less than 10, and less than 100 |
| D | no less than 100, and less than 10000 |

Unless otherwise specified, to the extent that there is a discrepancy between a depicted chemical structure of a compound provided herein and a chemical name of a compound provided herein, the chemical structure shall control.

A number of references have been cited, the disclosures of which are incorporated herein by reference in their entirety.

1-29. (canceled)

30. A compound selected from: 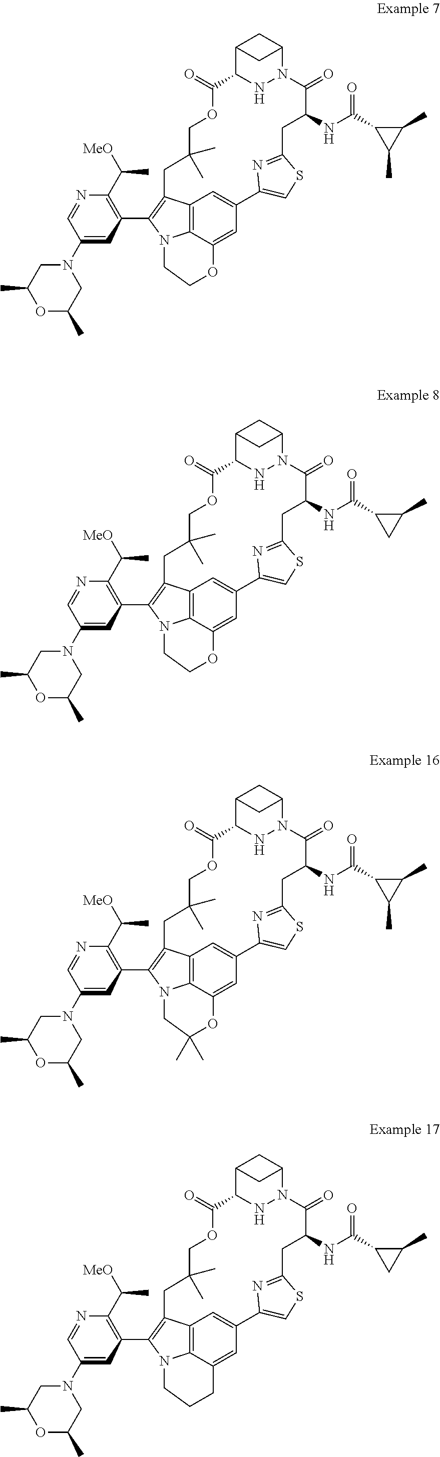 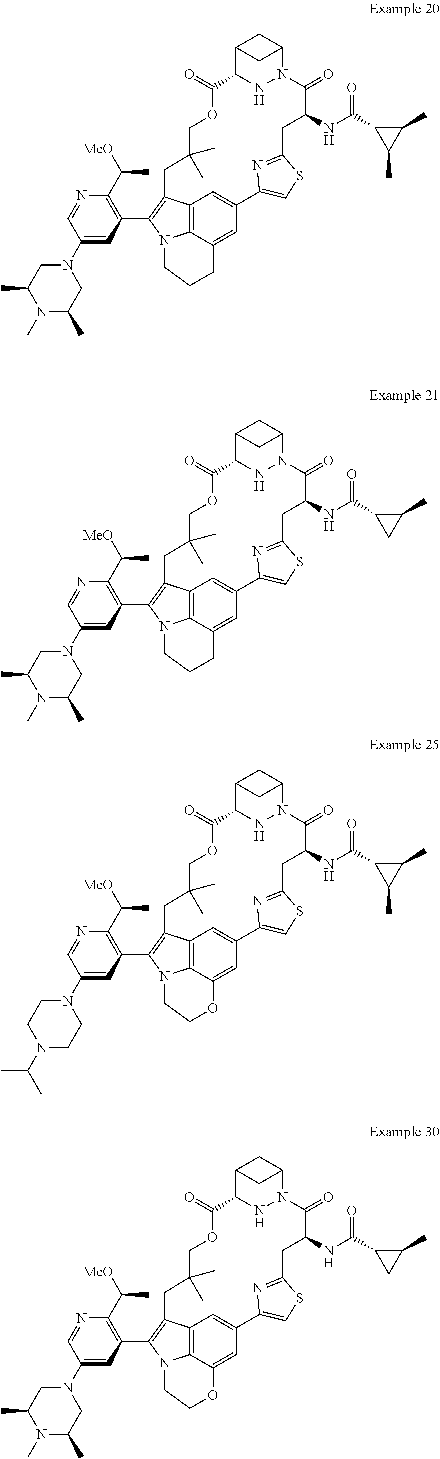 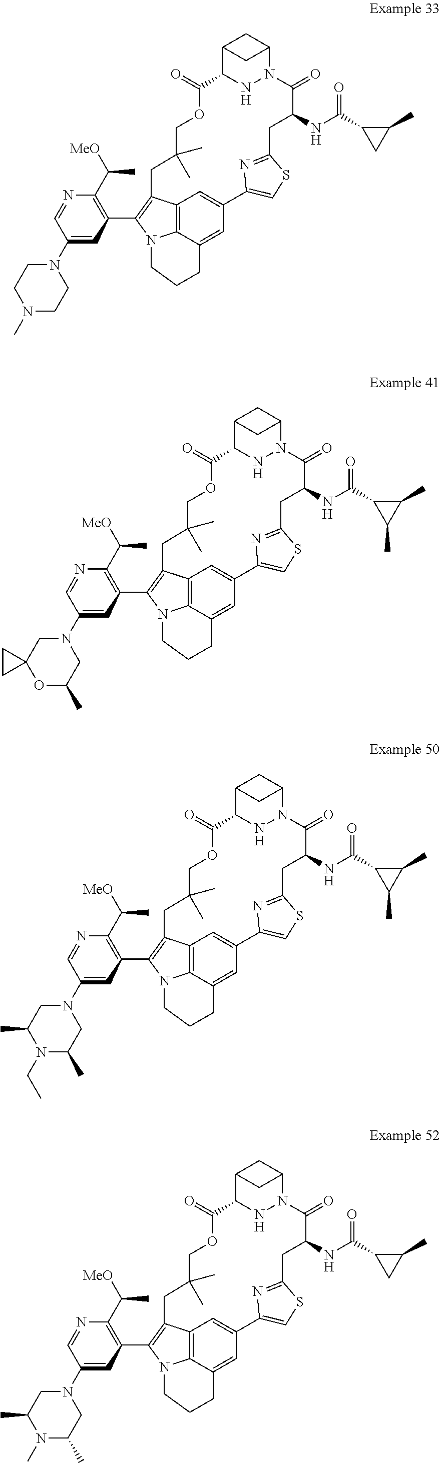 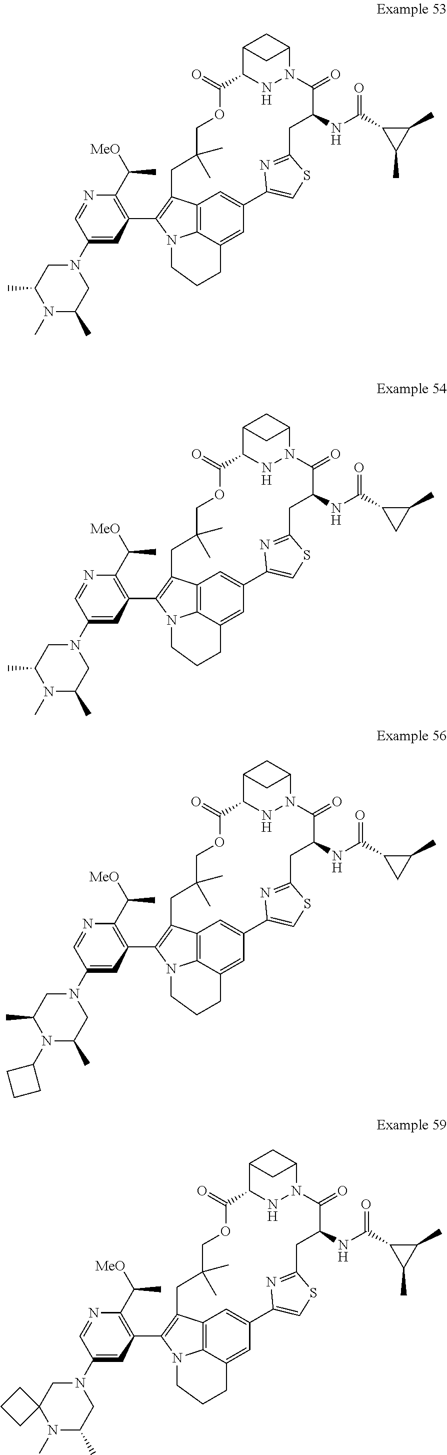 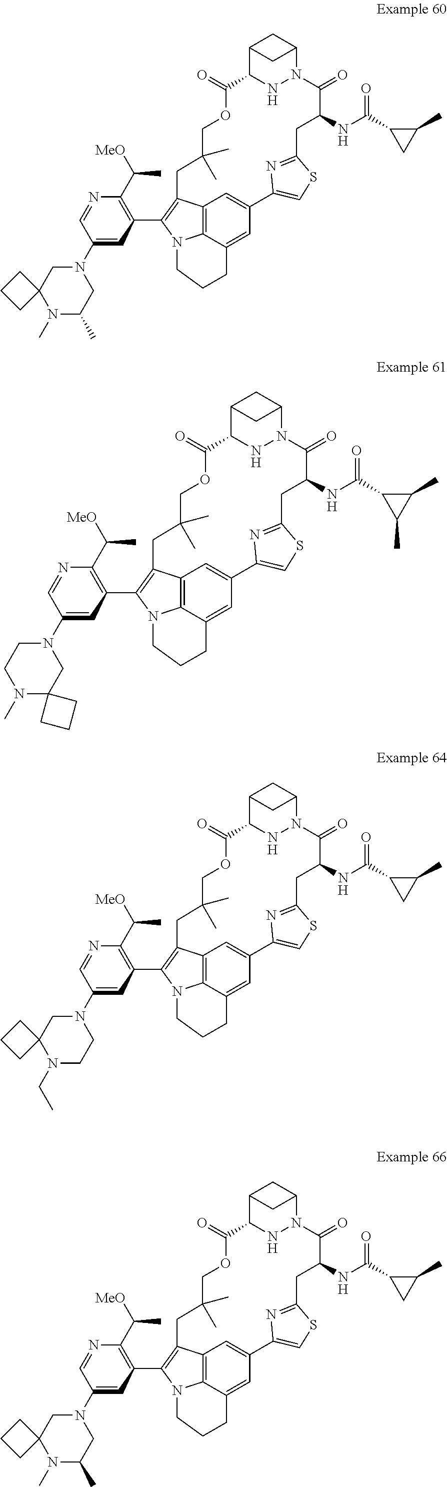
or a hydrate, solvate, or pharmaceutically acceptable salt thereof.

31. The compound of claim 30, wherein the compound is 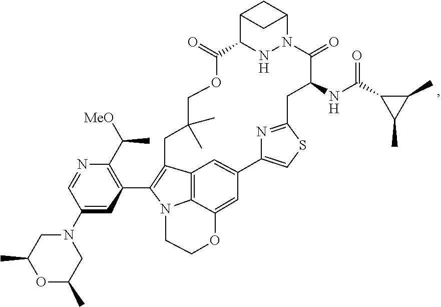
Example 7
or a hydrate, solvate, or pharmaceutically acceptable salt thereof.

32. The compound of claim 30, wherein the compound is 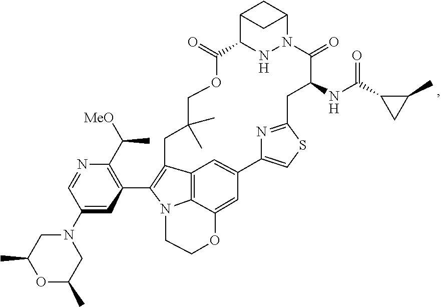
Example 8
or a hydrate, solvate, or pharmaceutically acceptable salt thereof.

33. The compound of claim 30, wherein the compound is 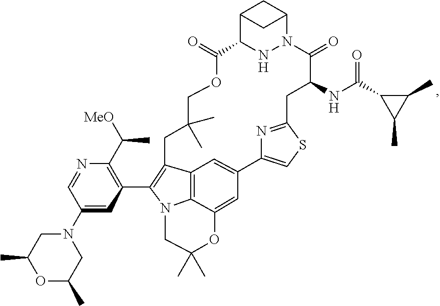
Example 16
or a hydrate, solvate, or pharmaceutically acceptable salt thereof.

34. The compound of claim 30, wherein the compound is 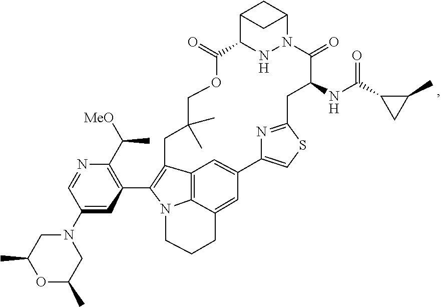
Example 17
or a hydrate, solvate, or pharmaceutically acceptable salt thereof.

35. The compound of claim 30, wherein the compound is 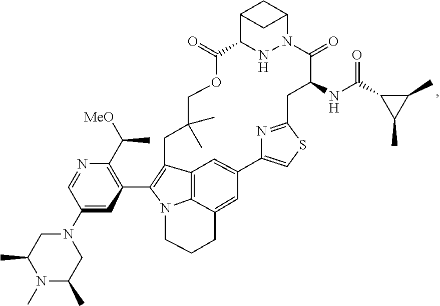
Example 20
or a hydrate, solvate, or pharmaceutically acceptable salt thereof.

36. The compound of claim 30, wherein the compound is 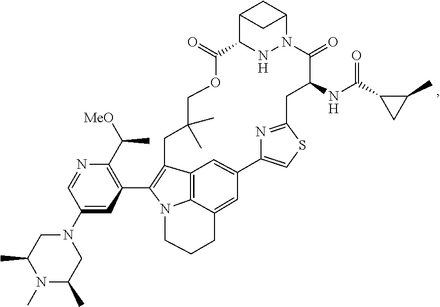
Example 21
or a hydrate, solvate, or pharmaceutically acceptable salt thereof.

37. The compound of claim 30, wherein the compound is 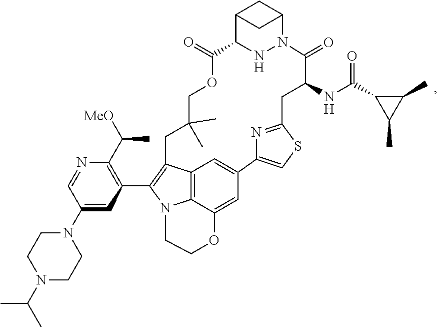
Example 25
or a hydrate, solvate, or pharmaceutically acceptable salt thereof.

38. The compound of claim 30, wherein the compound is 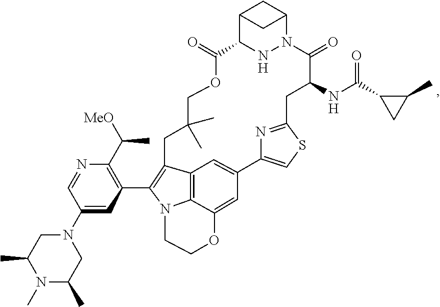
Example 30
or a hydrate, solvate, or pharmaceutically acceptable salt thereof.

39. The compound of claim 30, wherein the compound is 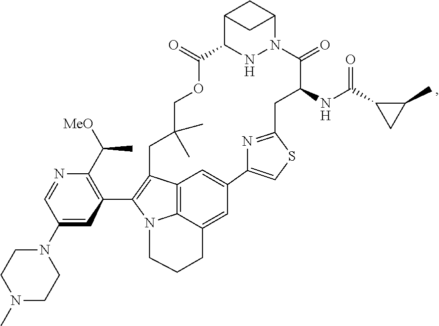
Example 33
or a hydrate, solvate, or pharmaceutically acceptable salt thereof.

40. The compound of claim 30, wherein the compound is 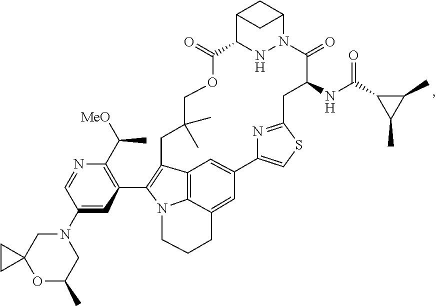
Example 41
or a hydrate, solvate, or pharmaceutically acceptable salt thereof.

41. The compound of claim 30, wherein the compound is 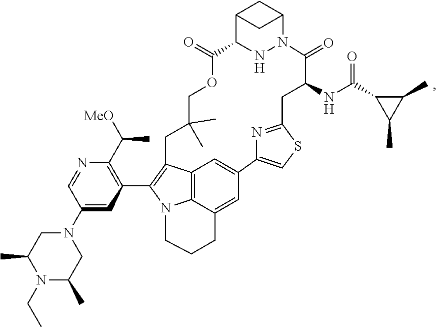
Example 50
or a hydrate, solvate, or pharmaceutically acceptable salt thereof.

42. The compound of claim 30, wherein the compound is 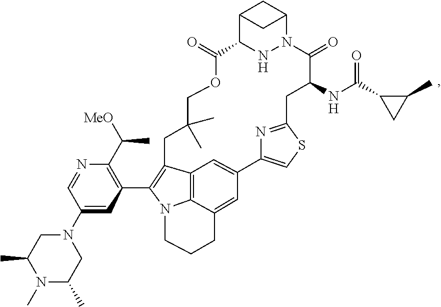
Example 52
or a hydrate, solvate, or pharmaceutically acceptable salt thereof.

43. The compound of claim 30, wherein the compound is 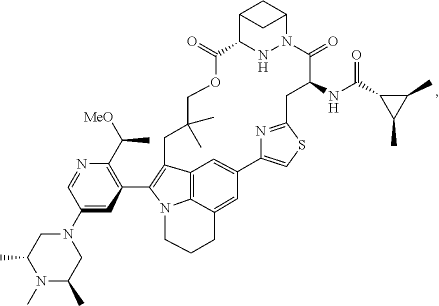
Example 53
or a hydrate, solvate, or pharmaceutically acceptable salt thereof.

44. The compound of claim 30, wherein the compound is 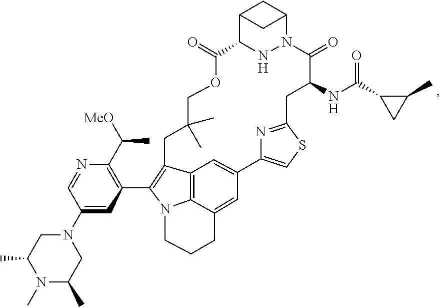
Example 54
or a hydrate, solvate, or pharmaceutically acceptable salt thereof.

45. The compound of claim 30, wherein the compound is 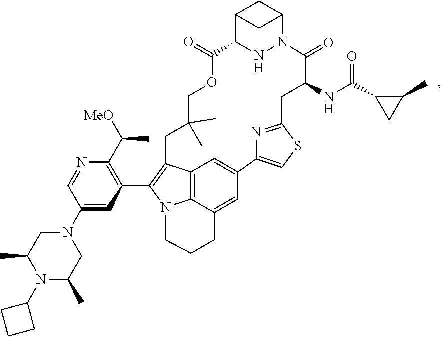
Example 56
or a hydrate, solvate, or pharmaceutically acceptable salt thereof.

46. The compound of claim 30, wherein the compound is 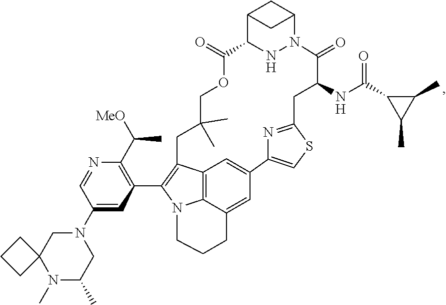
Example 59
or a hydrate, solvate, or pharmaceutically acceptable salt thereof.

47. The compound of claim 30, wherein the compound is 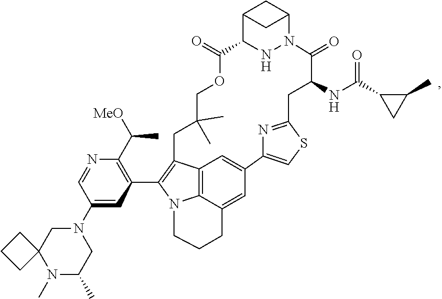
Example 60
or a hydrate, solvate, or pharmaceutically acceptable salt thereof.

48. The compound of claim 30, wherein the compound is 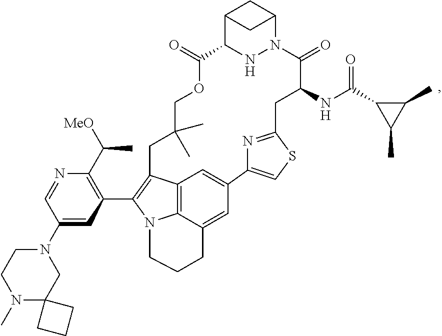
Example 61
or a hydrate, solvate, or pharmaceutically acceptable salt thereof.

49. The compound of claim 30, wherein the compound is 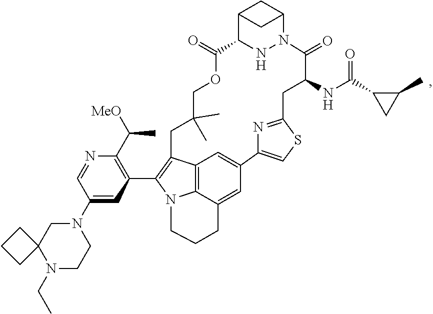
Example 64
or a hydrate, solvate, or pharmaceutically acceptable salt thereof.

50. The compound of claim 30, wherein the compound is 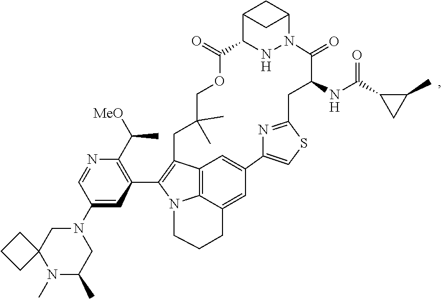
Example 66
or a hydrate, solvate, or pharmaceutically acceptable salt thereof.

51. A pharmaceutical composition comprising a compound of claim 30, or a hydrate, solvate, or pharmaceutically acceptable salt thereof, together with a pharmaceutically acceptable excipient.

52. A method of treating cancer in a subject in need thereof, the method comprising administering to the subject a therapeutically effective amount of a compound of claim 30, or a hydrate, solvate, or pharmaceutically acceptable salt thereof.

53. A method of treating a RAS protein-related disorder in a subject in need thereof, the method comprising administering to the subject a therapeutically effective amount of a compound of claim 30, or a hydrate, solvate, or pharmaceutically acceptable salt thereof.
